(19) United States
(12) Patent Application Publication
(10) Pub. No.: US 2024/0218339 A1
(43) Pub. Date: Jul. 4, 2024

(54) CLASS II, TYPE V CRISPR SYSTEMS

(71) Applicant: Metagenomi, Inc., Emeryville, CA (US)

(72) Inventors: Brian THOMAS, Berkeley, CA (US); Christopher BROWN, Albany, CA (US); Audra DEVOTO, Emeryville, CA (US); Cristina BUTTERFIELD, Oakland, CA (US); Lisa ALEXANDER, Albany, CA (US); Daniela S.A. GOLTSMAN, Oakland, CA (US)

(21) Appl. No.: 18/524,511

(22) Filed: Nov. 30, 2023

Related U.S. Application Data
(63) Continuation of application No. PCT/US22/31849, filed on Jun. 1, 2022.
(60) Provisional application No. 63/196,127, filed on Jun. 2, 2021.
(60) Provisional application No. 63/233,653, filed on Aug. 16, 2021.
(60) Provisional application No. 63/261,436, filed on Sep. 21, 2021.
(60) Provisional application No. 63/262,169, filed on Oct. 6, 2021.
(60) Provisional application No. 63/280,026, filed on Nov. 16, 2021.
(60) Provisional application No. 63/299,664, filed on Jan. 14, 2022.
(60) Provisional application No. 63/308,766, filed on Feb. 10, 2022.
(60) Provisional application No. 63/323,014, filed on Mar. 23, 2022.
(60) Provisional application No. 63/331,076, filed on Apr. 14, 2022.

(51) Int. Cl. C12N 9/22; C12N 15/11

(52) U.S. Cl. CPC C12N 9/22; C12N 15/111; C12N 2310/20

(57) ABSTRACT

Described herein are methods, compositions, and systems derived from uncultivated microorganisms useful for gene editing.

### CROSS-REFERENCE

This application is a continuation of International Application No. PCT/US2022/031849, filed Jun. 1, 2022, which claims the benefit of U.S. Provisional Application Nos: 63/196,127, filed Jun. 2, 2021; 63/233,653, filed Aug. 16, 2021; 63/261,436, filed Sep. 21, 2021; 63/262,169, filed Oct. 6, 2021; 63/280,026, filed Nov. 16, 2021; 63/299,664, filed Jan. 14, 2022; 63/308,766, filed Feb. 10, 2022; 63/323,014, filed Mar. 23, 2022; and 63/331,076, filed Apr. 14, 2022; each of which is incorporated by reference herein in their entireties. This application is related to PCT Application No. PCT/US21/21259, which is incorporated by reference herein in its entirety.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SEQUENCE LISTING

The instant application contains a Sequence Listing which has been submitted electronically in XML file format and is hereby incorporated by reference in its entirety. Said XML copy, created on Nov. 17, 2023, is named 55921-728_301_SL.xml and is 14,699,770 bytes in size.

### SUMMARY

In some aspects, the present disclosure provides for an engineered nuclease system comprising: (a) an endonuclease comprising a RuvC domain, wherein the endonuclease is derived from an uncultivated microorganism, and wherein the endonuclease is a Cas12a endonuclease; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the Cas12a endonuclease comprises the sequence GWxxxK. In some embodiments, the engineered guide RNA comprises UCUAC[N₃₋₅]GUAGAU (N₄). In some embodiments, the engineered guide RNA comprises CCUGC[N₄]GCAGG (N₃₋₄). In some aspects, the present disclosure provides for an engineered nuclease system comprising: (a) an endonuclease having at least 75% sequence identity to any one of SEQ ID NOs: 1-3470 or a variant thereof; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the endonuclease comprises a RuvCI, II, or III domain. In some embodiments, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to a RuvCI, II, or III domain of any one of SEQ ID NOs: 1-3470 or a variant thereof. In some embodiments, the RuvCI domain comprises a D catalytic residue. In some embodiments the RuvCII domain comprises an E catalytic residue. In some embodiments the RuvCIII domain comprises a D catalytic residue. In some embodiments, said RuvC domain does not have nuclease activity. In some embodiments, said endonuclease further comprises a WED II domain having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to a WED II domain of any one of SEQ ID NOs: 1-3470 or a variant thereof. In some aspects, the present disclosure provides for an engineered nuclease system comprising: (a) an endonuclease configured to bind to a protospacer adjacent motif (PAM) sequence comprising any one of SEQ ID NOs: 3890-3913 or any one of Sequence Numbers: A3863-A3889, wherein the endonuclease is a class 2, type V Cas endonuclease; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the endonuclease further comprises a zinc finger-like domain. In some embodiments, the guide RNA comprises a sequence with at least 80% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, and 3851-3857. In some aspects, the present disclosure provides for an engineered nuclease system comprising: (a) an engineered guide RNA comprising a sequence with at least 80% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, or 3851-3857, and (b) a class 2, type V Cas endonuclease configured to bind to the engineered guide RNA. In some embodiments, the endonuclease is configured to bind to a protospacer adjacent motif (PAM) sequence comprising any one of Sequence Numbers A3863-A3889 or any one of SEQ ID NOs: 3890-3913. In some embodiments, the guide RNA comprises a sequence complementary to a eukaryotic, fungal, plant, mammalian, or human genomic polynucleotide sequence. In some embodiments, the guide RNA is 30-250 nucleotides in length. In some embodiments, the endonuclease comprises one or more nuclear localization sequences (NLSs) proximal to an N- or C-terminus of the endonuclease. In some embodiments, the NLS comprises a sequence at least 80% identical to a sequence from the group consisting of SEQ ID NO: 3938-3953. In some embodiments, the endonuclease comprises at least one of the following mutations: S168R, E172R, N577R, or Y170R when a sequence of the endonuclease is optimally aligned to SEQ ID NO: 215. In some embodiments, the endonuclease comprises the mutations S168R and E172R when a sequence of the endonuclease is optimally aligned to SEQ ID NO: 215. In some embodiments, the endonuclease comprises the mutations N577R or Y170R when a sequence of the endonuclease is optimally aligned to SEQ ID NO: 215. In some embodiments, the endonuclease comprises the mutation S168R when a sequence of the endonuclease is optimally aligned to SEQ ID NO: 215. In some embodiments, the endonuclease does not comprise a mutation of E172, N577, or Y170. In some embodiments, the engineered nuclease system further comprises
a single- or double-stranded DNA repair template comprising from 5′ to 3′: a first homology arm comprising a sequence of at least 20 nucleotides 5′ to the target deoxyribonucleic acid sequence, a synthetic DNA sequence of at least 10 nucleotides, and a second homology arm comprising a sequence of at least 20 nucleotides 3′ to the target sequence. In some embodiments, the first or second homology arm comprises a sequence of at least 40, 80, 120, 150, 200, 300, 500, or 1,000 nucleotides. In some embodiments, the first and second homology arms are homologous to a genomic sequence of a prokaryote, bacteria, fungus, or eukaryote. In some embodiments, the single- or double-stranded DNA repair template comprises a transgene donor. In some embodiments, the engineered nuclease system further comprises a DNA repair template comprising a double-stranded DNA segment flanked by one or two single-stranded DNA segments. In some embodiments, single-stranded DNA segments are conjugated to the 5′ ends of the double-stranded DNA segment. In some embodiments, the single stranded DNA segments are conjugated to the 3′ ends of the double-stranded DNA segment. In some embodiments, the single-stranded DNA segments have a length from 4 to 10 nucleotide bases. In some embodiments, the single-stranded DNA segments have a nucleotide sequence complementary to a sequence within the spacer sequence. In some embodiments, the double-stranded DNA sequence comprises a barcode, an open reading frame, an enhancer, a promoter, a protein-coding sequence, a miRNA coding sequence, an RNA coding sequence, or a transgene. In some embodiments, the double-stranded DNA sequence is flanked by a nuclease cut site. In some embodiments, the nuclease cut site comprises a spacer and a PAM sequence. In some embodiments, the system further comprises a source of Mg²⁺. In some embodiments, the guide RNA comprises a hairpin comprising at least 8, at least 10, or at least 12 base-paired ribonucleotides. In some embodiments, the hairpin comprises 10 base-paired ribonucleotides. In some embodiments: (a) the endonuclease comprises a sequence at least 75%, 80%, or 90% identical to any one of SEQ ID NOs: 141, 215, 229, 261, or 1711-1721 or a variant thereof; and (b) the guide RNA structure comprises a sequence at least 80%, or 90% identical to the non-degenerate nucleotides of any one of SEQ ID NOs: 3608-3609, 3853, or 3851-3857. In some embodiments, the endonuclease is configured to bind to a PAM comprising any one of Sequence Numbers A3863-A3889 or any one of SEQ ID NOs: 3890-3913. In some embodiments, the endonuclease is configured to bind to a PAM comprising the sequence of YYn. In some embodiments, the sequence identity is determined by a BLASTP, CLUSTALW, MUSCLE, MAFFT algorithm, or a CLUSTALW algorithm with the Smith-Waterman homology search algorithm parameters. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for an engineered guide RNA comprising: (a) a DNA-targeting segment comprising a nucleotide sequence that is complementary to a target sequence in a target DNA molecule; and (b) a protein-binding segment comprising two complementary stretches of nucleotides that hybridize to form a double-stranded RNA (dsRNA) duplex, wherein the two complementary stretches of nucleotides are covalently linked to one another with intervening nucleotides, and wherein the engineered guide ribonucleic acid polynucleotide is capable of forming a complex with an endonuclease having at least 75% sequence identity to any one of SEQ ID NOs: 1-3470, and targeting the complex to the target sequence of the target DNA molecule. In some embodiments, the DNA-targeting segment is positioned 3′ of both of the two complementary stretches of nucleotides. In some embodiments, the protein binding segment comprises a sequence having at least 70%, at least 80%, or at least 90% identity to the non-degenerate nucleotides of SEQ ID NO: 3608-3609. In some embodiments, the double-stranded RNA (dsRNA) duplex comprises at least 5, at least 8, at least 10, or at least 12 ribonucleotides.

In some aspects, the present disclosure provides for a deoxyribonucleic acid polynucleotide encoding the engineered guide ribonucleic acid polynucleotide described herein.

In some aspects, the present disclosure provides for a nucleic acid comprising an engineered nucleic acid sequence optimized for expression in an organism, wherein the nucleic acid encodes a class 2, type V Cas endonuclease, and wherein the endonuclease is derived from an uncultivated microorganism, wherein the organism is not the uncultivated organism. In some embodiments, the endonuclease comprises a variant having at least 70% or at least 80% sequence identity to any one of SEQ ID NOs: 1-3470. In some embodiments, the endonuclease comprises a sequence encoding one or more nuclear localization sequences (NLSs) proximal to an N- or C-terminus of the endonuclease. In some embodiments, the NLS comprises a sequence selected from SEQ ID NOs: 3938-3953. In some embodiments, the NLS comprises SEQ ID NO: 3939. In some embodiments, the NLS is proximal to the N-terminus of the endonuclease. In some embodiments, the NLS comprises SEQ ID NO: 3938. In some embodiments, the NLS is proximal to the C-terminus of the endonuclease. In some embodiments, the organism is prokaryotic, bacterial, eukaryotic, fungal, plant, mammalian, rodent, or human.

In some aspects, the present disclosure provides for an engineered vector comprising a nucleic acid sequence encoding a class 2, type V Cas endonuclease or a Cas12a endonuclease, wherein the endonuclease is derived from an uncultivated microorganism.

In some aspects, the present disclosure provides for an engineered vector comprising a nucleic acid described herein.

In some aspects, the present disclosure provides for an engineered vector comprising a deoxyribonucleic acid polynucleotide described herein. In some embodiments, the vector is a plasmid, a minicircle, a CELiD, an adeno-associated virus (AAV) derived virion, a lentivirus, or an adenovirus.

In some aspects, the present disclosure provides for a cell comprising a vector described herein.

In some aspects, the present disclosure provides for a method of manufacturing an endonuclease, comprising cultivating any of the host cells described herein.

In some aspects, the present disclosure provides for a method for binding, cleaving, marking, or modifying a double-stranded deoxyribonucleic acid polynucleotide, comprising: (a) contacting the double-stranded deoxyribonucleic acid polynucleotide with a class 2, type V Cas endonuclease in complex with an engineered guide RNA configured to bind to the endonuclease and the double-stranded deoxyribonucleic acid polynucleotide; (b) wherein the double-stranded deoxyribonucleic acid polynucleotide comprises a protospacer adjacent motif (PAM); and (c) wherein the PAM comprises a sequence comprising any one of Sequence Numbers A3863-A3889 or any one of SEQ ID NOs: 3890-3913. In some embodiments, the double-stranded deoxyribonucleic acid polynucleotide comprises a first strand comprising a sequence complementary to a sequence of the engineered guide RNA and a second strand comprising the PAM. In some embodiments, the PAM is directly adjacent to the 5′ end of the sequence complementary to the sequence of the engineered guide RNA. In some embodiments, the PAM comprises a sequence of YYn. In some embodiments, the class 2, type V Cas endonuclease is derived from an uncultivated microorganism. In some embodiments, the double-stranded deoxyribonucleic acid polynucleotide is a eukaryotic, plant, fungal, mammalian, rodent, or human double-stranded deoxyribonucleic acid polynucleotide. In some embodiments, the method comprising delivering to the target nucleic acid locus the engineered nuclease system described herein, wherein the endonuclease is configured to form a complex with the engineered guide ribonucleic acid structure, and wherein the complex is configured such that upon binding of the complex to the target nucleic acid locus, the complex modifies the target nucleic acid locus. In some embodiments, modifying the target nucleic acid locus comprises binding, nicking, cleaving, or marking the target nucleic acid locus. In some embodiments, the target nucleic acid locus comprises deoxyribonucleic acid (DNA) or ribonucleic acid (RNA). In some embodiments, the target nucleic acid comprises genomic DNA, viral DNA, viral RNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, the cell is a primary cell. In some embodiments, the primary cell is a T cell. In some embodiments, the primary cell is a hematopoietic stem cell (HSC). In some embodiments, delivering the engineered nuclease system to the target nucleic acid locus comprises delivering the nucleic acid described herein or the vector described herein. In some embodiments, delivering the engineered nuclease system to the target nucleic acid locus comprises delivering a nucleic acid comprising an open reading frame encoding the endonuclease. In some embodiments, the nucleic acid comprises a promoter to which the open reading frame encoding the endonuclease is operably linked. In some embodiments, delivering the engineered nuclease system to the target nucleic acid locus comprises delivering a capped mRNA containing the open reading frame encoding the endonuclease. In some embodiments, delivering the engineered nuclease system to the target nucleic acid locus comprises delivering a translated polypeptide. In some embodiments, delivering the engineered nuclease system to the target nucleic acid locus comprises delivering a deoxyribonucleic acid (DNA) encoding the engineered guide RNA operably linked to a ribonucleic acid (RNA) pol III promoter. In some embodiments, the endonuclease induces a single-stranded break or a double-stranded break at or proximal to the target locus. In some embodiments, the endonuclease induces a staggered single stranded break within or 3′ to the target locus.

In some aspects, the present disclosure provides for a method of editing a TRAC locus in a cell, comprising contacting to the cell (a) an RNA-guided endonuclease; and (b) an engineered guide RNA, wherein the engineered guide RNA is configured to form a complex with the endonuclease and the engineered guide RNA comprises a spacer sequence configured to hybridize to a region of the TRAC locus, wherein the engineered guide RNA comprises a targeting sequence having at least 85% identity at least 18 consecutive nucleotides of any one of SEQ ID NOs: 4316-4369. In some embodiments, the RNA-guided nuclease is a Cas endonuclease. In some embodiments, the Cas endonuclease is a class 2, type V Cas endonuclease. In some embodiments, the class 2, type V Cas endonuclease comprises a RuvC domain comprising a RuvCI subdomain, a RuvCII subdomain, and a RuvCIII subdomain. In some embodiments, the class 2, type V Cas endonuclease comprises an endonuclease having at least 75% sequence identity to any one of SEQ ID NOs: 1-3470 or a variant thereof. In some embodiments, the engineered guide RNA further comprises a sequence with at least 80% sequence identity to at least 19 of the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, and 3851-3857. In some embodiments, the endonuclease comprises a sequence at least 75%, 80%, or 90% identical to any one of SEQ ID NOs: 141, 215, 229, 261, or 1711-1721 or a variant thereof. In some embodiments, the guide RNA structure comprises a sequence at least 80%, or at least 90% identical to at least 19 of the non-degenerate nucleotides of any one of SEQ ID NOs: 3608-3609, 3853, or 3851-3857. In some embodiments, the method further comprises contacting to the cell or introducing to the cell a donor nucleic acid comprising a cargo sequence flanked on a 3′ or 5′ end by sequence having at least 80% identity to any one of SEQ ID NOs: 4424 or 4425. In some embodiments, the cell is a peripheral blood mononuclear cell (PBMC). In some embodiments, the cell is a T-cell or a precursor thereof or a hematopoietic stem cell (HSC). In some embodiments, the cargo sequence comprises a sequence encoding a T-cell receptor polypeptide, a CAR-T polypeptide, or a fragment or derivative thereof. In some embodiments, the engineered guide RNA comprises a sequence having at least 80% identity to any one of SEQ ID NOs:4370-4423. In some embodiments, the engineered guide RNA comprises the nucleotide sequence of sgRNAs 1-54 from Table 5A comprising the corresponding chemical modifications listed in Table 5A. In some embodiments, the engineered guide RNA comprises a targeting sequence having at least 80% sequence identity to any one of SEQ ID NOs: 4334, 4350, or 4324. In some embodiments, the engineered guide RNA comprises a sequence having at least 80% sequence identity to any one of SEQ ID NOs: 4388, 4404, or 4378. In some embodiments, the engineered guide RNA comprises the nucleotide sequence of sgRNAs 9, 35, or 19 from Table 5A.

In some aspects, the present disclosure provides for an engineered nuclease system comprising: (a) an RNA-guided endonuclease; and (b) an engineered guide RNA, wherein the engineered guide RNA is configured to form a complex with the endonuclease and the engineered guide RNA comprises a spacer sequence configured to hybridize to a target nucleic acid sequence, wherein the engineered guide RNA comprises at least one of the following modifications: (i) a 2′-O methyl or a 2′-fluoro base modification of at least one nucleotide within the first 4 bases of the 5′ end of the engineered guide RNA or the last 4 bases of a 3′ end of the engineered guide RNA; (ii) a thiophosphate (PS) linkage between at least 2 of the first five bases of a 5′ end of the engineered guide RNA, or a thiophosphate linkage between at least two of the last five bases of a 3′ end of the engineered guide RNA; (iii) a thiophosphate linkage within a 3′ stem or a 5′ stem of the engineered guide RNA; (iv) a 2′-O methyl or 2′base modification within a 3′ stem or a 5′ stem of the engineered guide RNA; (v) a 2′-fluoro base modification of at least 7 bases of a spacer region of the engineered guide RNA; and (vi) a thiophosphate linkage within a loop region of the engineered guide RNA. In some embodiments, the engineered guide RNA comprises a 2′-O methyl or a 2′-fluoro base modification of at least one nucleotide within the first 5 bases of a 5′ end of the engineered guide RNA or the last 5 bases of a 3′ end of the engineered guide RNA. In some embodiments, the engineered guide RNA comprises a 2′-O methyl or a 2′-fluoro base modification at a 5′ end of the engineered guide RNA or a 3′ end of the engineered guide RNA. In some embodiments, the engineered guide RNA comprises a thiophosphate (PS) linkage between at least 2 of the first five bases of a 5′ end of the engineered guide RNA, or a thiophosphate linkage between at least two of the last five bases of a 3′ end of the engineered guide RNA. In some embodiments, the engineered guide RNA comprises a thiophosphate linkage within a 3′ stem or a 5′ stem of the engineered guide RNA. In some embodiments, the engineered guide RNA comprises a 2′-O methyl base modification within a 3′ stem or a 5′ stem of the engineered guide RNA. In some embodiments, the engineered guide RNA comprises a 2′-fluoro base modification of at least 7 bases of a spacer region of the engineered guide RNA. In some embodiments, the engineered guide RNA comprises a thiophosphate linkage within a loop region of the engineered guide RNA. In some embodiments, the engineered guide RNA comprises at least three 2′-O methyl or 2′-fluoro bases at the 5′ end of the engineered guide RNA, two thiophosphate linkages between the first 3 bases of the 5′ end of the engineered guide RNA, at least 4 2′-O methyl or 2′-fluoro bases at the 4′ end of the engineered guide RNA, and three thiophosphate linkages between the last three bases of the 3′ end of the engineered guide RNA. In some embodiments, the engineered guide RNA comprises at least two 2′-O-methyl bases and at least two thiophosphate linkages at a 5′ end of the engineered guide RNA and at least one 2′-O-methyl bases and at least one thiophosphate linkage at a 3′ end of the engineered guide RNA. In some embodiments, the engineered guide RNA comprises at least one 2′-O-methyl base in both the 3′ stem or the 5′ stem region of the engineered guide RNA. In some embodiments, the engineered guide RNA comprises at least one to at least fourteen 2′-fluoro bases in the spacer region excluding a seed region of the engineered guide RNA. In some embodiments, the engineered guide RNA comprises at least one 2′-O-methyl base in the 5′ stem region of the engineered guide RNA and at least one to at least fourteen 2′-fluoro bases in the spacer region excluding a seed region of the guide RNA. In some embodiments, the guide RNA comprises a spacer sequence targeting a VEGF-A gene. In some embodiments, the guide RNA comprises a spacer sequence having at least 80% identity to SEQ ID NO: 3985. In some embodiments, the guide RNA comprises the nucleotides of guide RNAs 1-7 from Table 7 comprising the chemical modifications listed in Table 7. In some embodiments, the RNA-guided nuclease is a Cas endonuclease. In some embodiments, the Cas endonuclease is a class 2, type V Cas endonuclease. In some embodiments, the class 2, type V Cas endonuclease comprises a RuvC domain comprising a RuvCI subdomain, a RuvCII subdomain, and a RuvCIII subdomain. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1721 or a variant thereof. In some embodiments, the engineered guide RNA comprises a sequence with at least 80% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, and 3851-3857. In some embodiments, the engineered guide RNA comprises a sequence with at least 80% sequence identity to the non-degenerate nucleotides of any one of SEQ ID NOs: 3608-3609, 3853, or 3851-3857.

In some aspects, the present disclosure provides for a host cell comprising an open reading frame encoding a heterologous endonuclease having at least 75% sequence identity to any one of SEQ ID NOs: 1-3470 or a variant thereof. In some embodiments, the endonuclease has at least 75% sequence identity to any one of SEQ ID NOs: 141, 215, 229, 261, or 1711-1721, or a variant thereof. In some embodiments, the host cell is an E. coli cell or a mammalian cell. In some embodiments, the host cell is an E. coli cell, wherein the E. coli cell is a λDE3 lysogen or the E. coli cell is a BL21(DE3) strain. In some embodiments, the E. coli cell has an ompT Ion genotype. In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof. In some embodiments, the open reading frame comprises a sequence encoding an affinity tag linked in-frame to a sequence encoding the endonuclease. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the endonuclease via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, the open reading frame is codon-optimized for expression in the host cell. In some embodiments, the open reading frame is provided on a vector. In some embodiments, the open reading frame is integrated into a genome of the host cell.

In some aspects, the present disclosure provides for a culture comprising any of the host cells described herein in compatible liquid medium.

In some aspects, the present disclosure provides for a method of producing an endonuclease, comprising cultivating any of the host cells described herein in compatible growth medium. In some embodiments, the method further comprises inducing expression of the endonuclease. In some embodiments, the inducing expression of the nuclease is by addition of an additional chemical agent or an increased amount of a nutrient, or by temperature increase or decrease. In some embodiments, an additional chemical agent or an increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises isolating the endonuclease. In some embodiments, the isolating comprises subjecting the protein extract to IMAC, ion-exchange chromatography, anion exchange chromatography, or cation exchange chromatography. In some embodiments, the open reading frame comprises a sequence encoding an affinity tag linked in-frame to a sequence encoding the endonuclease. In some embodiments, the affinity tag is linked in-frame to the sequence encoding the endonuclease via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the affinity tag by contacting a protease corresponding to the protease cleavage site to the endonuclease. In some embodiments, the affinity tag is an IMAC affinity tag. In some embodiments, the method further comprises performing subtractive IMAC affinity chromatography to remove the affinity tag from a composition comprising the endonuclease.

In some aspects, the present disclosure provides for a system comprising (a) a class 2, Type V-A Cas endonuclease configured to bind a 3- or 4-nucleotide PAM sequence, wherein the endonuclease has increased cleavage activity relative to sMbCas12a; and (b) an engineered guide RNA, wherein the engineered guide RNA is configured to form a complex with the class 2, Type V-A Cas endonuclease and the engineered guide RNA comprises a spacer sequence configured to hybridize to a target nucleic acid comprising a target nucleic acid sequence. In some embodiments, the cleavage activity is measured in vitro by introducing the endonucleases alongside compatible guide RNAs to cells comprising the target nucleic acid and detecting cleavage of the target nucleic acid sequence in the cells. In some embodiments, the class 2, Type V-A Cas endonuclease comprises a sequence having at least 75% identity to any one of 215-225 or a variant thereof. In some embodiments, the engineered guide RNA comprises a sequence having at least 80% identity to the non-degenerate nucleotides of SEQ ID NO: 3609. In some embodiments, the target nucleic acid further comprises a YYN PAM sequence proximal to the target nucleic acid sequence. In some embodiments, the class 2, Type V-A Cas endonuclease has at least about 5%, 10%, 20%, 30%, 40%, 50%, 60%, 70%, 80%, 90%, 100%, or 200%, or more increased activity relative to sMbCas12a.

In some aspects, the present disclosure provides for a system comprising: (a) a class 2, Type V-A′ Cas endonuclease; and (b) an engineered guide RNA, wherein the engineered guide RNA comprises a sequence having at least 80% identity to about 19 to about 25 or about 19 to about 31 consecutive nucleotides of a natural effector repeat sequence of a class 2, Type V Cas endonuclease. In some embodiments, the natural effector repeat sequence is any one of SEQ ID NOs: 3560-3572. In some embodiments, the class 2, Type V-A′ Cas endonuclease has at least 75% identity to SEQ ID NO: 126.

In some aspects, the present disclosure provides for a system comprising: (a) a class 2, Type V-L endonuclease, and (b) an engineered guide RNA, wherein the engineered guide RNA comprises a sequence having at least 80% identity to about 19 to about 25 or about 19 to about 31 consecutive nucleotides of a natural effector repeat sequence of a class 2, Type V Cas endonuclease. In some embodiments, the class 2, Type V-L endonuclease has at least 75% sequence identity to any one of SEQ ID NOs: 793-1163.

In some aspects, the present disclosure provides for a method of disrupting the VEGF-A locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the VEGF-A locus, wherein the engineered guide RNA comprises a targeting sequence having at least 80% identity to SEQ ID NO: 3985; or wherein the engineered guide RNA comprises the nucleotide sequence of any one of guide RNAs 1-7 from Table 7 In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1721 or a variant thereof. In some embodiments, the engineered guide RNA comprises a sequence with at least 80% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, and 3851-3857. In some embodiments, the engineered guide RNA comprises a sequence with at least 80% sequence identity to the non-degenerate nucleotides of any one of SEQ ID NOs: 3608-3609, 3853, or 3851-3857.

In some aspects, the present disclosure provides for a method of disrupting a locus in a cell, comprising contacting to the cell a composition comprising: (a) a class 2, type V Cas endonuclease having at least 75% identity to any one of SEQ ID NOs: 215-225 or a variant thereof; and (b) an engineered guide RNA, wherein the engineered guide RNA is configured to form a complex with the endonuclease and the engineered guide RNA comprises a spacer sequence configured to hybridize to a region of the locus, wherein the class 2, type V Cas endonuclease has at least equivalent cleavage activity to spCas9 in the cell. In some embodiments, the cleavage activity is measured in vitro by introducing the endonucleases alongside compatible guide RNAs to cells comprising the target nucleic acid and detecting cleavage of the target nucleic acid sequence in the cells. In some embodiments, the composition comprises 20 pmoles or less of the class 2, type V Cas endonuclease. In some embodiments, the composition comprises 1 pmol or less of the class 2, type V Cas endonuclease. In some aspects, the present disclosure provides for a method of disrupting a CD38 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the CD38 locus, wherein the engineered guide RNA is configured to hybridize to a sequence having at least 20-22 consecutive nucleotides having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4466-4503 and 5686; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4428-4465 and 5685. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4466, 4467, 4468, 4479, 4484, 4490, 4492, 4493, 4495, 4498. In some embodiments, the engineered guide RNA comprises a nucleotide sequence having at least 80% identity to any one of SEQ ID NOs: 4428, 4429, 4430, 4436, 4441, 4446, 4452, 4454, 4455, 4460, or 4461. In some embodiments, the cell is a eukaryotic cell, T-cell, hematopoietic stem cell, or precursor thereof. In some aspects, the present disclosure provides for a method of disrupting a TIGIT locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the TIGIT locus, wherein the engineered guide RNA is configured to hybridize to a sequence having at least 20-22 consecutive nucleotides having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4521-4537; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4504-4520. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4521, 4527, 4528, 4535, or 4536. In some embodiments, the engineered guide RNA comprises a nucleotide sequence having at least 80% identity to any one of SEQ ID NOs: 4504, 4510, 4511, 4518, or 4519. In some embodiments, the cell is a eukaryotic cell, T-cell, hematopoietic stem cell, or precursor thereof.

In some aspects, the present disclosure provides for a method of disrupting an AAVS1 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the AAVS1 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4569-4599; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4538-4568. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4574, 4577, 4578, 4579, 4582, 4584, 4585, 4586, 4587, 4589, 4590, 4591, 4592, 4593, 4595, 4596, or 4598. In some embodiments, the engineered guide RNA comprises a nucleotide sequence having at least 80% identity to any one of SEQ ID NOs: 4543, 4546, 4547, 4548, 4551, 4553, 4554, 4555, 4556, 4558, 4559, 4560, 4561, 4562, 4565, or 4567. In some embodiments, the cell is a eukaryotic cell, T-cell, hematopoietic stem cell, hepatocyte, or precursor thereof.

In some aspects, the present disclosure provides for a method of disrupting a B2M locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the B2M locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4676-4751; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4600-4675. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857 and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4676, 4678-4687, 4690, 4692, 4698-4707, 4720-4723, 4725-4726, 4732-4733, 4736-4737, 4741, or 4750-4751. In some embodiments, the engineered guide RNA comprises a nucleotide sequence having at least 80% identity to any one of SEQ ID NOs: 4600, 4602-4611, 4614, 4616, 4622-4631, 4644-4647, 4649-4650, 4656-4657, 4660-4661, 4665, or 4674-4675. In some embodiments, the cell is a eukaryotic cell, T-cell, hematopoietic stem cell, or precursor thereof.

In some aspects, the present disclosure provides for a method of disrupting a CD2 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the CD2 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4837-4921; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4752-4836. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4837, 4844, 4845, 4848, 4857-4858, 4883, 4887, 4892-4893, 4904-4909, 4914, 4916, or 4918. In some embodiments, the engineered guide RNA has at least 80% sequence identity to any of the guide RNAs from Table 14E that target any one of SEQ ID NOs: 4837, 4844, 4845, 4848, 4857-4858, 4883, 4887, 4892-4893, 4904-4909, 4914, 4916, or 4918. In some embodiments, the cell is a eukaryotic cell, T-cell, hematopoietic stem cell, or precursor thereof.

In some aspects, the present disclosure provides for a method of disrupting a CD5 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the CD5 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4946-4969; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4922-4945. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4946-4947, 4949, 4951, 4957-4960, 4963, 4967, or 4969. In some embodiments, the engineered guide RNA has at least 80% sequence identity to any of the guide RNAs from Table 14F that target any one of SEQ ID NOs: 4946-4947, 4949, 4951, 4957-4960, 4963, 4967, or 4969. In some embodiments, the cell is a eukaryotic cell, T-cell, hematopoietic stem cell, or precursor thereof.

In some aspects, the present disclosure provides for a method of disrupting a mouse TRAC locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the mouse TRAC locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5126-5195, 5682, or 5684; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5056-5125, 5681, or 5683. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5126-5130, 5133-5143, 5147-5150, 5172-5173, 5184-5189, or 5192-5194. In some embodiments, the engineered guide RNA has at least 80% sequence identity to any of the guide RNAs from Table 14G that target any one of SEQ ID NOs: 5126-5130, 5133-5143, 5147-5150, 5172-5173, 5184-5189, or 5192-5194. In some embodiments, the cell is a eukaryotic cell, T-cell, hematopoietic stem cell, or precursor thereof.

In some aspects, the present disclosure provides for a method of disrupting a mouse TRBC1 or TRBC2 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the mouse TRBC1 or TRBC2 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5211-5225 or 5247-5267; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5196-5210 or 5226-5246. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5211, 5213-5215, 5217, 5221, 5223, 5247, 5249-5250, 5252-5253, 5258-5259, or 5264. In some embodiments, the engineered guide RNA has at least 80% sequence identity to any of the guide RNAs from Table 14H that target any one of SEQ ID NOs: 5211, 5213-5215, 5217, 5221, 5223, 5247, 5249-5250, 5252-5253, 5258-5259, or 5264. In some embodiments, the cell is a eukaryotic cell, T-cell, hematopoietic stem cell, or precursor thereof.

In some aspects, the present disclosure provides for a method of disrupting a human TRBC1 or TRBC2 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the human TRBC1 or TRBC2 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at last about 99% sequence identity to any one of SEQ ID NOs: 5661-5679; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5642-5660. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5661-5663, 5672-5675, or 5678. In some embodiments, the engineered guide RNA has at least 80% sequence identity to any of the guide RNAs from Table 141 that target any one of SEQ ID NOs: 5661-5663, 5672-5675, or 5678. In some embodiments, the cell is a eukaryotic cell, T-cell, hematopoietic stem cell, or precursor thereof.

In some aspects, the present disclosure provides for a method of disrupting an HPRT locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the HPRT locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5562-5641; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5482-5561. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5562-5564 or 5568. In some embodiments, the engineered guide RNA has at least 80% sequence identity to any of the guide RNAs from Table 14J that target any one of SEQ ID NOs: 5562-5564 or 5568. In some embodiments, the cell is a eukaryotic cell, hepatocyte, T-cell, hematopoietic stem cell, or precursor thereof.

In some aspects, the present disclosure provides for a method of disrupting an APO-A1 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the APO-A1 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5861-5874; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5847-5860. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5861-5866 or 5868-5869. In some embodiments, the engineered guide RNA has at least 80% sequence identity to any of the guide RNAs from Table 43A that target any one of SEQ ID NOs: 5861-5866 or 5868-5869. In some embodiments, the cell is a eukaryotic cell, hepatocyte, T-cell, hematopoietic stem cell, or precursor thereof.

In some aspects, the present disclosure provides for a method of disrupting an ANGPTL3 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the ANGPTL3 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5953-6030; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5875-5952. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5955-5963, 5968-5975, 5979-5987, 5989-5993, 5997, 5999, 6003-6010, 6014-6016, 6024-6025, or 6027-6030. In some embodiments, the engineered guide RNA has at least 80% sequence identity to any of the guide RNAs from Table 43B that target any one of SEQ ID NOs: 5955-5963, 5968-5975, 5979-5987, 5989-5993, 5997, 5999, 6003-6010, 6014-6016, 6024-6025, or 6027-6030. In some embodiments, the cell is a eukaryotic cell, hepatocyte, T-cell, hematopoietic stem cell, or precursor thereof.

In some aspects, the present disclosure provides for a method of disrupting a human Rosa26 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the human Rosa26 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5013-5055; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4970-5012. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof.

In some aspects, the present disclosure provides for a method of disrupting a FAS locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the FAS locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5367-5465; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5268-5366. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof.

In some aspects, the present disclosure provides for a method of disrupting a PD-1 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the PD-1 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5474-5481; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5466-5473. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof.

In some aspects, the present disclosure provides for an engineered nuclease system comprising: (a) an endonuclease having at least 75% sequence identity to any one of SEQ ID NOs: 215 or a variant thereof; and (b) an engineered guide RNA, wherein the engineered guide RNA is configured to form a complex with the endonuclease and the engineered guide RNA comprises a spacer sequence configured to hybridize to a target nucleic acid sequence, wherein the system has reduced immunogenicity when administered to a human subject compared to an equivalent system comprising a Cas9 enzyme. In some embodiments, the Cas9 enzyme is an SpCas9 enzyme. In some embodiments, the guide RNA comprises a sequence with at least 80% sequence identity to the non-degenerate nucleotides of SEQ ID NO: 3609. In some embodiments, the immunogenicity is antibody immunogenicity.

An aspect of the present disclosure provides for a method of disrupting a mouse HAO-1 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the mouse HAO-1 locus, wherein the engineered guide RNA comprises the nucleotides of guide RNAs mH29-1_37, mH29-15_37, mH29-29_37 from Table 25 comprising the nucleotide modifications described in Table 25; or wherein the engineered guide RNA comprises any one of SEQ ID NOs: 4184-4225. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof. In some embodiments, the engineered guide RNA comprises the nucleotides of guide RNAs mH29-15_37 or mH29-29_37 from Table 25 comprising the nucleotide modifications described in Table 25. In some embodiments, the method further comprises disrupting expression of glycolate oxidase from the HAO-1 locus.

In some aspects, the present disclosure provides for a method of disrupting a human TRAC locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the human TRAC locus, wherein the engineered guide RNA comprises the nucleotides of MG29-1-TRAC-sgRNA-35 from Table 28B comprising the nucleotide modifications described in Table 28B. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof.

An aspect of the present disclosure provides for a method of disrupting an albumin locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the albumin locus, wherein the engineered guide RNA comprises the nucleotides of mA1b298-37, mA1b2912-37, mA1b2918-37, or mA1b298-34 from Table 29 comprising the nucleotide modifications described in Table 29; or wherein the engineered guide RNA comprises the nucleotides of mA1b29-8-44, mA1b29-8-50, mA1b29-8-50b, mA1b29-8-51b, mA1b29-8-52b, mA1b29-8-53b, or mA1b29-8-54b comprising the nucleotide modifications described in Table 46. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof. In some embodiments, the engineered guide RNA comprises the nucleotides of mA1b298-37, mA1b2912-37, mA1b2918-37, or mA1b298-34 from Table 29 comprising the nucleotide modifications described in Table 29.

In some aspects, the present disclosure provides for an engineered guide RNA comprising: (a) a DNA-targeting segment comprising a nucleotide sequence that is complementary to a target sequence in a target DNA molecule; and (b) a protein-binding segment configured to bind to a class 2, type V Cas endonuclease, and wherein the guide RNA comprises a nucleotide modification pattern depicted in any one of SEQ ID NOs: 5695-5701 in Table 34. In some embodiments, the guide RNA comprises mA1b29-8-44, mA1b29-8-50, mA1b29-8-37, or mA1b29-12-44. In some embodiments, the guide RNA comprises hH29-4_50, hH29-21_50, hH29-23_50, hH29-41_50, hH29-4_50b, hH29-21_50b, hH29-23_50b, or hH29-41_50b, mH29-1-50, mH29-15-50, mH29-29-50, mH29-1-50b, mH29-15-50b, or mH29-29-50b. In some embodiments, the DNA-targeting segment is configured to hybridize to an HAO-1 gene or an albumin gene. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the class 2, type V Cas endonuclease comprises an endonuclease having at least 75% sequence identity to SEQ ID NO: 215.

In some aspects, the present disclosure provides for an engineered nuclease system comprising: (a) an endonuclease having at least 75% sequence identity to any one of SEQ ID NOs: 1-3470 or a variant thereof, or a nucleotide sequence encoding the enonuclease; and (b) a polynucleotide sequence encoding a CRISPR array, wherein the CRISPR array is configured to be processed by the endonuclease to an engineered guide RNA, wherein the engineered guide RNA is configured to form a complex with the endonuclease and the engineered guide RNA comprises a spacer sequence configured to hybridize to a target nucleic acid sequence, wherein the spacer sequence is configured to hybridize to an albumin gene. In some embodiments, the polynucleotide sequence comprises a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 5712. In some embodiments, the endonuclease comprises an endonuclease having at least 75% sequence identity to any one of SEQ ID NOs: 141, 215, 229, 261, or 1711-1722 or a variant thereof. In some embodiments, the endonuclease comprises an endonuclease having at least 75% sequence identity to SEQ ID NO: 215.

In some aspects, the present disclosure provides for an engineered nuclease system comprising: (a) an endonuclease having at least 75% sequence identity to SEQ ID NOs: 470 or a variant thereof; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the engineered guide RNA comprises a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 6031. In some embodiments, the endonuclease is configured to be selective for a 5′ PAM sequence comprising a sequence of YTn.

In some aspects, the present disclosure provides for an engineered nuclease system comprising: (a) an endonuclease having at least at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 2824, 2841, or 2896, or a variant thereof; and (b) an engineered guide RNA, wherein the engineered guide RNA is configured to form a complex with the endonuclease and the engineered guide RNA comprises a spacer sequence configured to hybridize to a target nucleic acid sequence, wherein the engineered guide RNA comprises a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 6033, 6034, or 6035. In some embodiments, the endonuclease has at least 80% sequence identity to SEQ ID NO: 2824 and the engineered guide RNA has at least 80% sequence identity to SEQ ID NO: 6033. In some embodiments, the endonuclease has at least 80% sequence identity to SEQ ID NO: 2841 and the engineered guide RNA has at least 80% sequence identity to SEQ ID NO: 6034. In some embodiments, the endonuclease has at least 80% sequence identity to SEQ ID NO: 2896 and the engineered guide RNA has at least 80% sequence identity to SEQ ID NO: 6035. In some embodiments, the endonuclease is configured to be selective for a 5′ PAM sequence comprising any one of Sequence Numbers: A6037-A6039.

In some aspects, the present disclosure provides for a lipid nanoparticle comprising: (a) any of the endonucleases described herein; (b) any of the engineered guide RNAs described herein: (c) a cationic lipid; (d) a sterol; (e) a neutral lipid; and (f) a PEG-modified lipid. In some embodiments, the cationic lipid comprises C12-200, the sterol comprises cholesterol, the neutral lipid comprises DOPE, or the PEG-modified lipid comprises DMG-PEG2000. In some embodiments, the cationic lipid comprises any of the cationic lipids depicted in FIG. 109.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts example organizations of CRISPR/Cas loci of different classes and types that were previously documented before this disclosure.

FIG. 2 depicts environmental distribution of MG nucleases described herein. Protein length is shown for representatives of the MG29 protein family. Shades of circle indicates the environment or environment type from which each protein was identified (dark gray circle indicates high temperature environment source; light gray circle indicates non-high temperature environment source). N/A denotes the type of environment the sample was collected from is unknown.

FIG. 3 depicts the number of predicted catalytic residues present in MG nucleases detected from sample types described herein (e.g. FIG.). Protein length is shown for representatives of the MG29 protein family. The number of catalytic residues that were predicted for each protein are indicated in the figure legend (3.0 residues). The first, second and third catalytic residues are located in the RuvCI domain, the RuvCII domain and the RuvCIII domain, respectively.

FIGS. 4A and 4B show the diversity of CRISPR Type V-A effectors. FIG. 4A depicts per family distribution of taxonomic classification of contigs encoding the novel Type V-A effectors. FIG. 4B depicts the phylogenetic gene tree inferred from an alignment of 119 novel and 89 reference Type V effector sequences. MG families are denoted in parentheses. PAM requirements for active nucleases are outlined with boxes associated with the family. Non-Type V-A reference sequences were used to root the tree (*MG61 family requires a crRNA with an alternative stem-loop sequence).

FIGS. 5A, 5B, 5C, and 5D provide various characteristic information about nucleases described herein. FIG. 5A depicts the per family distribution of effector protein length and the type of sample; FIG. 5B shows the presence of RuvC catalytic residues. FIG. 5C shows the number of CRISPR arrays having various repeat motifs. FIG. 5D depicts the per family distribution of repeat motifs.

FIGS. 6A and 6B depict multiple sequence alignment of catalytic and PAM interacting regions in Type V-A sequences. Francisella novicida Cas12a (FnCas12) is a reference sequence. Other reference sequences are Acidaminococcus sp. (AsCas12a), Moraxella bovoculi (MbCas12a), and Lachnospiraceae bacterium ND2006 (LbCas12a). FIG. 6A shows blocks of conservation around the DED catalytic residues in RuvC-I (left), RuvC-II (middle), and RuvC-III (right) regions. FIG. 6B shows WED-II and PAM interacting regions containing residues involved in PAM recognition and interaction. The grey boxes underneath the FnCas12a sequence identify the domains. Darker boxes in the alignments indicate increased sequence identity. Black boxes over the FnCas12a sequence indicate catalytic residues (and positions) of the reference sequence. Grey boxes indicate domains in the reference sequence at the top of the alignment (FnCas12a). Black boxes indicate catalytic residues (and positions) of the reference sequence.

FIGS. 7A and 7B depicts Type V-A and associated V-A′ effectors. FIG. 7A shows Type V-A (MG26-1) and V-A′ (MG26-2) indicated by arrows pointing in the direction of transcription. The CRISPR array is indicated by a gray bar. Predicted domains for each protein in the contig are indicated by boxes. FIG. 7B shows sequence alignments of Type V-A′ MG26-2 and AsCas12a reference sequence. Top: RuvC-I domain. Middle: region containing the RuvC-I and RuvC-II catalytic residues. Bottom: region containing the RuvC-III catalytic residue. Catalytic residues are indicated by squares.

FIG. 8 depicts a schematic representation of the structure of a sgRNA and a target DNA in a ternary complex with AacC2C1 (see Yang, Hui, Pu Gao, Kanagalaghatta R. Rajashankar, and Dinshaw J. Patel. 2016. “PAM-Dependent Target DNA Recognition and Cleavage by C2c1 CRISPR-Cas Endonuclease.” Cell 167 (7): 1814-28.e12 which is incorporated by reference herein in its entirety).

FIG. 9 depicts the effects of mutations or truncations in the R-AR domains of an sgRNA on AacC2c1-mediated cleavage of linear plasmid DNA; WT, wild-type sgRNA. The mutant nucleotides within sgRNA (lanes 1-5) are highlighted in the left panel. Δ15: 15 nt deleted from the sgRNA R-AR 1 region. Δ12: 12 nt have been removed from the sgRNA J2/4 R-AR 1 region (see Liu, Liang, Peng Chen, Min Wang, Xueyan Li, Jiuyu Wang, Maolu Yin, and Yanli Wang. 2017. “C2c1-sgRNA Complex Structure Reveals RNA-Guided DNA Cleavage Mechanism.” Molecular Cell 65 (2): 310-22 which is incorporated by reference herein in its entirety).

FIGS. 10A, 10B, and 10C demonstrate that the CRISPR RNA (crRNA) structure is conserved among Type V-A systems. FIG. 10A shows the fold structure of the reference crRNA sequence in the LbCpf1 system. FIG. 10B shows multiple sequence alignment of CRISPR repeats associated with novel Type V-A systems. The LbCpf1 processing site is indicated with a black bar. FIG. 10C shows the fold structure of MG61-2 putative crRNA with an alternative stem-loop motif CCUGC[N₃₋₄]GCAGG. FIG. 10D shows multiple sequence alignment of CRISPR repeats with the alternative repeat motif sequence. The processing sites and loop are indicated.

FIG. 11 depicts a predicted structure of a guide RNA utilized herein (SEQ ID NO: 3608).

FIG. 12 depicts predicted structures of corresponding sgRNAs of MG enzymes described herein (clockwise, SEQ ID NOs: 3636, 3637, 3641, 3640).

FIG. 13 depicts predicted structures of corresponding sgRNAs of MG enzymes described herein (clockwise, SEQ ID NOs: 3644, 3645, 3649, 3648).

FIG. 14 depicts predicted structures of corresponding sgRNAs of MG enzymes described herein (clockwise, SEQ ID NOs: 3652, 3653, 3657, 3656).

FIG. 15 depicts predicted structures of corresponding sgRNAs of MG enzymes described herein (clockwise, SEQ ID NOs: 3660, 3661, 3665, 3664).

FIG. 16 depicts predicted structures of corresponding sgRNAs of MG enzymes described herein (clockwise, SEQ ID NOs: 3666, 3667, 3672, 3671).

FIGS. 17A and 17B depict an agarose gel showing the results of PAM vector library cleavage in the presence of TXTL extracts containing various MG family nucleases and their corresponding tracrRNA or sgRNAs (as described in Example 12). FIG. 17A shows lane 1: ladder. The bands are, from top to bottom, 766, 500, 350, 300, 350, 200, 150, 100, 75, 50; lane 2: 28-1+MGcrRNA spacer1 (SEQ ID NOs: 141+3860); lane 3: 29-1+MGcrRNA spacer1 (SEQ ID NOs: 215+3860); lane 4: 30-1+MGcrRNA spacer1 (SEQ ID NOs:226+3860); lane 5: 31-1+MGcrRNA spacer1 (SEQ ID NOs: 229+3860); lane 6: 32-1+MGcrRNA spacer1 (SEQ ID NOs: 261+3860); lane 7: ladder. FIG. 17B shows lane 1: ladder; lane 2: LbaCas12a+LbaCas12a crRNA spacer2; lane 3: LbaCas12a+MGcrRNA spacer2; lane 4: Apo 13-1; lane 5: 28-1+MGcrRNA spacer2 (SEQ ID NOs: 141+3861); lane 6: 29-1+MGcrRNA spacer2 (SEQ ID NOs: 215+3861); lane 7: 30-1+MGcrRNA spacer2 (SEQ ID NOs: 226+3861); lane 8: 31-1+MGcrRNA spacer2 (SEQ ID NOs: 229+3861); lane 9: 32-1+MGcrRNA spacer2 (SEQ ID NOs: 261+3861)

FIGS. 18A, 18B, 18C, 18D, and 18E provide data showing that type V-A effectors described herein are active nucleases. FIG. 18A depicts seqLogo representations of PAM sequences determined for three nucleases described herein. FIG. 18B shows a boxplot of plasmid transfection activity assays inferred from frequency of indel edits for active nucleases. The boundaries of the boxplots indicate first and third quartile values. The mean is indicated with an “x” and the median is represented by the midline within each box. FIG. 18C shows plasmid transfection editing frequencies at four target sites for MG29-1 and AsCas12a. One side-by-side experiment with AsCas12a was done. FIG. 18D shows plasmid and RNP editing activity for nuclease MG29-1 at 14 target loci with either TIN or CCN PAMs. FIG. 18E shows the editing profile of nuclease MG29-1 from RNP transfection assays. One side-by-side experiment with AsCas12a was done. Editing frequency and profile experiments for MG29-1 were done in duplicate. The bar plots FIG. 18C and FIG. 18D show mean editing frequency with one standard deviation error bar.

FIG. 19 depicts in cell indel formation generated by transfection of HEK cells with MG29-1 constructs described in Example 12 alongside their corresponding sgRNAs containing various different targeting sequences targeting various locations in the human genome.

FIG. 20 depicts seqLogo representations of PAM sequences of specific MG family enzymes derived via NGS as described herein (as described in Example 13).

FIG. 21 depict seqLogo representations of PAM sequences of specific MG family enzymes derived via NGS as described herein (top to bottom, Sequence Numbers: A3865, A3867, A3872).

FIG. 22 depict seqLogo representations of PAM sequences derived via NGS as described herein (top to bottom, Sequence Numbers: A3879, A3880, A3881).

FIG. 23 depict seqLogo representations of PAM sequences derived via NGS as described herein (top to bottom, Sequence Numbers: A3883, A3884, A3885).

FIG. 24 depict seqLogo representations of PAM sequences derived via NGS as described herein (Sequence Number: A3882).

FIG. 25 depicts in cell indel formation generated by transfection of HEK cells with MG31-1 constructs described in Example 14 alongside their corresponding sgRNAs containing various different targeting sequences targeting various locations in the human genome.

FIGS. 26A, 26B, and 26C shows the biochemical characterization of Type V-A nucleases. FIG. 26A shows PCR of cleavage products with adaptors ligated to their ends shows activity of nucleases described herein and Cpf1 (positive control) when bound to a universal crRNA. Expected cleavage product band labeled with an arrow. FIG. 26B shows PCR of cleavage products with adaptors ligated to their ends show activity of nucleases described herein when bound to their native crRNA. Cleavage product band indicated with an arrow. FIG. 26C shows analysis of the NGS cut sites shows cleavage on the target strand at position 22, sometimes with less frequent cleavage after 21 or 23 nt.

FIGS. 27A and 27B depict multiple sequence alignments of Type V-L nucleases described herein, showing (FIG. 27A) an example locus organization for a Type V-L nuclease, and (FIG. 27B) a multiple sequence alignment. Regions containing putative RuvC-III domains are shown as light grey rectangles. Putative RuvC catalytic residues are shown as small dark grey rectangles above each sequence. Putative single-guide RNA binding sequences are small white rectangles, putative scissile phosphate binding sites are indicated by black rectangles above sequences, and residues predicted to disrupt base stacking near the scissile phosphate in the target sequence are indicated by small medium-grey rectangles above sequences.

FIG. 28 shows a Type V-L candidate labeled MG60 as an example locus organization alongside an effector repeat structure and a phylogenetic tree showing the location of the enzyme in the Type V families.

FIG. 29 shows examples of smaller Type V effectors one of which may be labeled as MG70.

FIG. 30 shows characteristic information of MG70 as described herein. Depicted is an example locus organization alongside a phylogenetic tree illustrating the location of these enzymes in the Type V family.

FIG. 31 shows another example of a small Type V effector MG81 as described herein. Depicted is an example locus organization alongside a phylogenetic tree illustrating the location of these enzymes in the Type V family.

FIG. 32 shows that the activity individual enzymes of Type V effector families identified herein (e.g. MG20, MG60, MG70, other) is maintained over a variety of different enzyme lengths (e.g. 400-1200 AA). Light dots (True) indicate active enzymes while dark dots (unknown) indicate untested enzymes.

FIG. 33 depicts sequence conservation of MG nucleases described herein. The black bars indicate putative RuvC catalytic residues.

FIG. 34 and FIG. 35 depict an enlarged version of multiple sequence alignments in FIG. 33 of regions of the MG nucleases described herein containing putative RuvC catalytic residues (dark-grey rectangles), scissile phosphate-binding residues (black rectangles), and residues predicted to disrupt base stacking adjacent to the scissile phosphate (light-grey rectangles).

FIG. 36 depicts the regions of the MG nucleases described herein containing putative RuvC-III domain & catalytic residues.

FIG. 37 depicts regions of the MG nucleases containing putative single-guide RNA-binding residues (white rectangles above sequences).

FIG. 38 depicts multiple protein sequence alignment of representatives from several MG type V Families. Shown are conserved regions containing portions of the RuvC domain predicted to be involved in nuclease activity. Predicted catalytic residues are highlighted.

FIG. 39 shows a screen of the TRAC locus for MG29-1 gene editing. A bar graph shows indel creation resulting from transfection of MG29-1 with 54 separate guide RNAs targeting the TRAC locus in primary human T cells. The corresponding guide RNAs depicted in the figure are identified in SEQ ID NOs: 4316-4423.

FIG. 40 depicts the optimization of MG29-1 editing at TRAC. A bar graph shows indel creation resulting from transfection of MG29-1 (at the indicated concentrations) with the four best 22 nt guide RNAs from FIG. 39 (9, 19, 25, and 35). Legend: MG29-1 9 is MG29-1 effector (SEQ ID NO: 215) and Guide 9 (SEQ ID NO: 4378), MG29-1 19 is MG29-1 effector (SEQ ID NO: 215) and Guide 19 (SEQ ID NO: 4388), MG29-1 25 is MG29-1 effector (SEQ ID NO: 215) and Guide 25 (SEQ ID NO: 4394), and MG29-1 35 is MG29-1 effector (SEQ ID NO: 215) and Guide 35 (SEQ ID NO: 4404).

FIG. 41 depicts the optimization of dose and guide length for MG29-1 editing at TRAC. Line graphs show the indel creation resulting from transfection of MG29-1 and either guide RNA #19 (SEQ ID NO: 4388) or guide RNA #35 (SEQ ID NO: 4404). Three different doses of nuclease/guide RNA were used. For each dose, six different guide lengths were tested, successive one-nucleotide 3′ truncations of SEQ ID NOs: 4388 and 4404. The guides used in FIG. 39 and FIG. 40 are the 22 nt-long spacer-containing guides in this case.

FIG. 42 shows a correlation of indel generation at TRAC and loss of the T cell receptor expression in the Experiment of Example 22.

FIG. 43 depicts targeted transgene integration at TRAC stimulated by MG29-1 cleavage. Cells receiving transgene donor alone by AAV infection retain TCR expression and lack CAR expression; cells transfected with MG29-1 RNPs and infected with 100,000 vg (vector genomes) of a CAR transgene donor lose TCR expression and gain CAR expression. Shown are FACS plots of CAR antigen binding vs TCR expression for cells transfected with AAV alone containing the CAR-T-containing donor sequence (“AAV”); AAV containing the CAR-T-containing donor sequence with MG29-1 enzyme and sgRNA 19 (SEQ ID NO: 4388) (“AAV+MG29-1-19-22” comprising a 22 nucleotide spacer), or AAV containing the CAR-T-containing donor sequence with MG29-1 enzyme and sgRNA 35 (SEQ ID NO: 4404) (“AAV+MG29-1-35-22” comprising a 22 nucleotide spacer).

FIG. 44 shows MG29-1 gene editing at TRAC in hematopoietic stem cells. A bar graph shows the extent of indel creation at TRAC after transfection with MG29-1-9-22 (“MG29-1 9”; MG29-1 plus guide RNA #19) and MG29-1-35-22 (“MG29-1 35”; MG29-1 plus guide RNA #35) compared to mock-transfected cells.

FIG. 45 shows the refinement of the MG29-1 PAM based on analysis of gene editing outcomes in cells. Guide RNAs were designed using a 5′-NTTN-3′ PAM sequence and then sorted according to the gene editing activity observed. The identity of the underlined base (the 5′-proximal N) is shown for each bin. All of the guides with activity greater than 10% had a T at this position in the genomic DNA indicating that the MG29-1 PAM may be best described as 5′-TTTN-3′. The statistical significance of the over-representation of T at this position is shown for each bin.

FIG. 46 depicts the analysis of gene editing activity versus the base composition of MG29-1 spacer sequences. A bar graph shows experimental data illustrating a relationship between GC content (%) and indel frequency (“high” signifies >50% indels (N=4); “medium” signifies 10-50% indels (N=15); “>1%” signifies 1-5% indels (N=12); “<1%” signifies less than 1% indels (N=82)).

FIG. 47 depicts MG29-1 guide RNA chemical modifications. The bar graph shows the consequences of modifications from Table 7 on VEGF-A editing activity relative to an unmodified guide RNA (sample #1).

FIG. 48 depicts a dose titration of a variously chemically modified MG29-1 RNA. The bar graphs show indel generation after transfection of RNPs with guides using modification patterns 1, 4, 5, 7, and 8. RNPs doses were 126 pmol MG29-1 and 160 pmol guide RNA or as indicated. Full dose (A), ¼th (B), ⅛th (C), 1/16th (D), and 1/32nd (E).

FIG. 49 depicts a plasmid map of pMG450 (MG29-1 nuclease protein in lac inducible tac promoter E. coli BL21 expression vector.

FIG. 50 depicts the indel profile of MG29-1 with spacer mALb29-1-8 (SEQ ID NO: 3999) compared to spCas9 with a guide targeting mouse albumin intron 1.

FIG. 51 is a representative indel profile of MG29-1 with a guide targeting mouse albumin intron 1 determined by next generation sequencing (approximately 15,000 total reads analyzed) as in Example 29.

FIG. 52 shows the editing efficiency of MG29-1 compared to spCas9 in mouse liver cell line Hepa1-6 nucleofected with RNP as in Example 29.

FIGS. 53A, 53B, 53C, and 53D show the editing efficiencies in mammalian cells of MG29-1 variants with single and double amino acid substitutions compared to wild type MG29-1. FIG. 53A depicts editing efficiency in Hepa 1-6 cells transfected with plasmids codifying for MG29-1 WT or mutant versions. FIG. 53B depicts Editing efficiency in Hepa 1-6 cells transfected with mRNA encoding WT or S168R at various concentrations. FIG. 53C depicts the editing efficiency in Hepa 1-6 cells transfected with mRNA codifying versions of MG29-1 with single or double amino acid substitutions. FIG. 53D depicts the editing efficiency in Hepa 1-6 and HEK293T cells transfected with MG29-1 WT vs S168R in combination with 13 guides. 12 guides correspond to guides in Table 7. Guide “35 (TRAC)” is a guide targeting the human locus TRAC.

FIG. 54 shows the predicted secondary structure of the MG29-1 guide mA1b29-1-8.

FIG. 55 shows the impact of chemical modifications of the MG29-1 sgRNA sequence upon the stability of the sgRNA in whole cell extracts of mammalian cells.

FIGS. 56A, 56B, and 56C show the use of sequencing to identify the cut site on the target strand in an in vitro reaction performed with MG29-1 protein, a guide RNA, and an appropriate template. FIG. 56A shows the distance of the cut position from the PAM in nucleotides as determined by next generation sequencing. FIG. 56B shows the use of Sanger Sequencing to define the MG29-1 cut site on the target strand. FIG. 56C shows the use of Sanger Sequencing to define the MG29-1 cut site on the non-target strand. Run-off Sanger sequencing was performed on in vitro reactions containing MG29-1, a guide, and an appropriate template to evaluate the cleavage of both strands. The cleavage site on the target strand is position 23 which is consistent with the NGS data in FIG. 56A which shows cleavage at 21-23 bases. The “A” peak at the end of the sequence is due to polymerase run off and is expected. The cleavage site on the non-target strand can be seen in the reverse read in which the expected terminating base is “T”. The marked spot (line) shows cleavage at position 17 from the PAM and then the terminal T. However, there is a mixed T signal at positions 18, 19, and 20 from the PAM suggesting variable cleavage on this strand at positions 17, 18, and 19.

FIG. 57 depicts the gene editing outcomes at the DNA level for CD38. S. pyogenes (Spy) Cas9 guides for CD38 and TRAC are shown at right.

FIG. 58 depicts the gene editing outcomes at the phenotypic level for CD38.

FIG. 59 depicts the gene editing outcomes at the DNA level for TIGIT.

FIG. 60 depicts the gene editing outcomes at the DNA level for AAVS1.

FIG. 61 depicts the gene editing outcomes at the DNA level for B2M.

FIG. 62 depicts the gene editing outcomes at the DNA level for CD2.

FIG. 63 depicts the gene editing outcomes at the DNA level for CD5.

FIG. 64A depicts the gene editing outcomes at the DNA level for mouse TRAC. FIG. 64B depicts the flow cytometry results for gene editing of mouse TRAC.

FIG. 65 depicts the percentage of TRAC knock-out versus the percentage of indels.

FIG. 66A depicts the gene editing outcomes at the DNA level for mouse TRBC1. FIG. 66B depicts the gene editing outcomes at the DNA level for mouse TRBC2. FIG. 66C depicts the flow cytometry results for gene editing of human TRBC1/2.

FIG. 67 depicts the gene editing outcomes at the DNA level for HPRT.

FIG. 68 depicts the activity of chemically modified guides in Hepa1-6 cells when delivered as mRNA and gRNA using lipofectamine Messenger Max.

FIG. 69 depicts the stability of guides modified with modification 44 versus end-modified or unmodified guides.

FIGS. 70A and 70B depict a comparison of guide stability for Type II and Type V systems. FIG. 70A depicts stability data for unmodified guides. FIG. 70B depicts stability data for guides with 5′ and 3′ end modifications.

FIG. 71 depicts the predicted secondary structures of MG29-1 (Type V) and MG3-6/3-4 (Type II) guide RNA. The backbone (tracr) portion is shown.

FIG. 72 depicts the stability of guide mA1b298-34 compared to mA1b298-37 in cell lysates from Hepa1-6 cells.

FIG. 73 depicts the editing efficiency of MG29-1 in mouse liver following in vivo delivery.

FIG. 74 depicts analysis of gene-editing outcomes by NGS for mRNA electroporation in T cells.

FIG. 75 depicts analysis of gene-editing outcomes by NGS for chemically modified guides.

FIG. 76 depicts ELISA results from a screen performed at a serum dilution of 1:50 to detect antibodies against MG29-1 (n=50). Tetanus toxoid was used as the positive control due to wide-spread vaccination against this antigen. Serum samples above the dashed line were considered antibody-positive; the line represents the mean absorbance of the negative control (human albumin) plus two standard deviations from the mean. *P<0.05, **P<0.01, ****P<0.0001 as determined by an unpaired Student's t-test; ns, not significant.

FIG. 77 depicts HAO-1 editing efficiency in mouse liver as measured by NGS. Each point represents an individual mouse.

FIGS. 78A-B depict the effects of HAO-1 editing on glycolate oxidase (GO) protein levels in mouse liver as evaluated by Western Blot. 10 μg of total protein was loaded for each sample.

FIG. 79 depicts Western Blot analysis of glycolate oxidase (GO) protein levels in an untreated mouse compared to two individual mice treated with lipid nanoparticles (LNPs) encapsulating MG29-1 mRNA and either guide mH29-1_37 or mH29-5_37. Three different amounts of total liver protein (40 μg, 20 μg, and 10 μg) from each mouse were loaded on the gel then processed for detection of the mouse glycolate oxidase protein.

FIG. 80 depicts an example INDEL profile for MG29-1 and an sgRNA targeting the HAO-1 gene in mouse liver. The sample was taken from mouse #17 (treated with a lipid nanoparticle encapsulating mH29-29_37 and MG29-1 WT mRNA).

FIG. 81 depicts the gene editing outcomes at the DNA level for TRAC in human peripheral blood B cells.

FIG. 82 depicts the gene editing outcomes at the DNA level for TRAC in hematopoietic stem cells.

FIG. 83 depicts the gene editing outcomes at the DNA level for TRAC in induced pluripotent stem cells (iPSCs).

FIG. 84 depicts the results of in vivo genome editing with MG29-1 as quantified by next generation sequencing (NGS).

FIG. 85 depicts an example INDEL profile generated by the MG29-1 nuclease and guide 298-37 as measured by next generation sequencing (NGS).

FIG. 86 depicts spacer length optimization for MG29-1 guides targeting two loci.

FIG. 87 depicts in vitro stability of sgRNAs for MG29-1 and MG3-6/3-4.

FIG. 88 depicts the predicted secondary structures of the backbone parts of the guide RNA for MG29-1 and MG3-6/3-4.

FIG. 89 depicts the predicted secondary structure of an MG3-6/3-4 guide with a spacer targeting mouse albumin.

FIG. 90 depicts the predicted secondary structure of an MG29-1 guide with stem-loop 1 from MG3-6/3-4 added to the 5′ end.

FIG. 91 depicts the editing efficiency of MG29-1 with mouse albumin guide 8 with chemistries 44 or 50 in Hepa1-6 cells by mRNA transfection or RNP nucleofection.

FIG. 92 depicts editing in the liver of mice after dosing with LNP encapsulating MG29-1 mRNA and one of four different guide RNAs.

FIG. 93 depicts the predicted secondary structure of the RNA molecule mA1b29-g8-37-array.

FIG. 94 depicts a plot showing editing efficiency in the whole liver of mice at 5 days after intravenous injection of LNP encapsulating one of: (1) MG29-1 mRNA and guide mA1b29-8-50 (mA29-8-50) at three different doses; (2) spCas9 mRNA and guide mA1bR2 at three different doses; or (3) PBS buffer (Control). Each circle represents a single mouse and the bars indicate the mean and standard deviation.

FIG. 95 depicts editing activity in Hep3B cells transfected with MG29-1 mRNA and 6 sgRNA targeting human HAO-1.

FIG. 96 depicts editing Activity of 4 MG29-1 sgRNA targeting human HAO-1 in HuH7 and Hep3B cells transfected with Ribonuclear Protein Complexes.

FIG. 97 depicts editing activity of MG29-1 with sgRNA targeting the human HAO-1 gene in primary human hepatocytes.

FIG. 98A depicts a representative indel profile for MG29-1 sgRNAs hH29-4-37 and hH29-21-37 in Primary Human Hepatocytes. FIG. 98B depicts a representative indel profile for MG29-1 sgRNAs hH29-23-37 and hH29-41-37 in Primary Human Hepatocytes.

FIG. 99 depicts the activity of MG29-1 guide RNAs with 22 nucleotide or 20 nucleotide spacers targeting mouse HAO-1 in mouse liver.

FIG. 100 depicts the impact of the mRNA/guide RNA ratio and separate or co-formulation on editing efficiency in mouse liver.

FIG. 101 depicts the evaluation of MG29-1 guide chemistries on editing activity in the liver of mice after in vivo delivery in LNP.

FIG. 102 depicts the gene-editing outcomes at the DNA level for APO-A1 in Hepa1-6 cells.

FIG. 103 depicts the gene-editing outcomes at the DNA level for ANGPTL3 in Hepa1-6 cells.

FIG. 104A-E depicts in vitro characterization of MG55-43. FIG. 104A shows the genomic region in the vicinity of the MG55-43 nuclease. Genes are represented by orange arrows. The gene encoding the candidate nuclease includes a “putative transposase DNA-binding domain”. The CRISPR array is represented by repeats and spacers. The predicted tracrRNA is shown as an arrow between the array and the nuclease and labeled “Predicted-trimmed-TracrRNA-CM2”. FIG. 104B shows active single guide RNA design (tracrRNA and repeat sequences connected by a tetraloop). The color of the nitrogenated bases corresponds to the probability of base pairing of that base, where red is high probability and blue is low probability.

FIG. 104C shows an agarose gel showing in vitro cleavage of plasmid target DNA library with the sgRNA and two different spacers (U67 and U40). Lanes that are not related to the MG55-43 nuclease are not shown. FIG. 104D shows sequence logo showing the MG55-43 PAM sequence.

FIG. 104E shows a histogram showing the cut site position in the spacer sequence tested with MG55-43.

FIG. 105A-C depicts examples of genomic regions encoding MG91 nucleases. Genes are represented by arrows with genes encoding candidate nuclease labeled as such. The CRISPR array is represented by repeats and spacers. Intergenic regions potentially encoding active tracrRNAs are highlighted as bars labeled IG # or Intergenic region #.

FIG. 106 depicts multiple sequence alignments of intergenic region nucleotide sequences potentially containing tracrRNAs. Green bars on top indicate a high degree of similarity among the sequence of the intergenic regions. FIG. 106A shows intergenic region 2 in the vicinity of the MG91-15 nuclease and its relatives. FIG. 106B shows intergenic region 2 in the vicinity of the MG91-32 nuclease and its relatives. FIG. 106C shows intergenic region 2 in the vicinity of the MG91-87 nuclease and its relatives.

FIG. 107A-D depicts single guide RNA designs and in vitro cleavage assay results. For the single guide RNA designs (tracrRNA and repeat sequences), the color of the bases corresponds to the probability of base pairing of that base, where red is high probability and blue is low probability. FIG. 107A depicts MG91-15 sgRNA1, FIG. 107B depicts MG91-32 sgRNA1, and FIG. 107C depicts MG91-87 sgRNA1. FIG. 107D depicts an agarose gel showing in vitro cleavage of plasmid target DNA library with different sgRNA designs (sgRNA1 and sgRNA2) and two different spacers (U67 and U40). Lanes that are not related to these nucleases are not shown.

FIG. 108A-F depicts sequence logos of predicted PAM sequence and histograms showing cut site position. FIGS. 108A, 108B, and 108C depict sequence logos showing the PAM sequences of MG91-15, MG91-32, and MG91-87, respectively. FIGS. 108D, 108E, and 108F depict histograms showing the cut site positions in the spacer sequences tested with MG91-15, MG91-32, and MG91-87, respectively.

FIG. 109 depicts structures of example cationic lipids that can be used in lipid nanoparticles described herein.

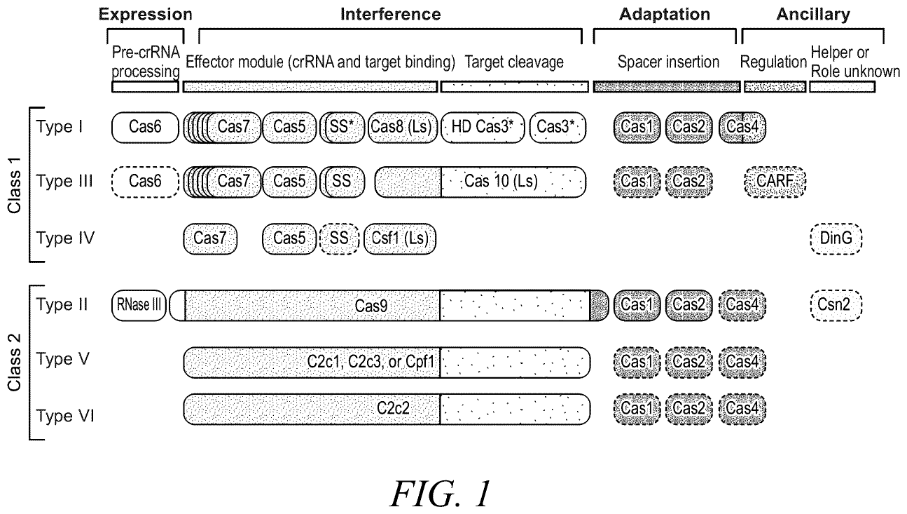

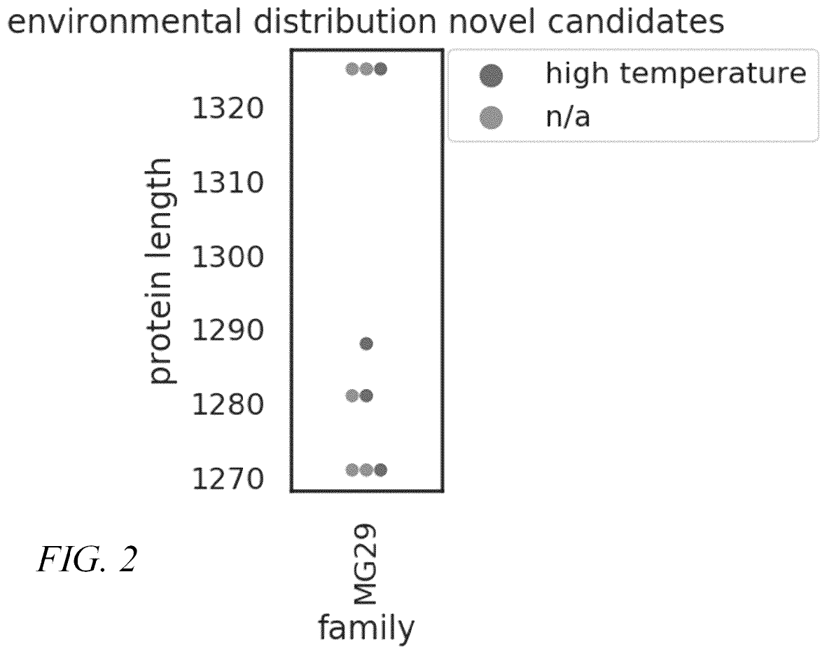

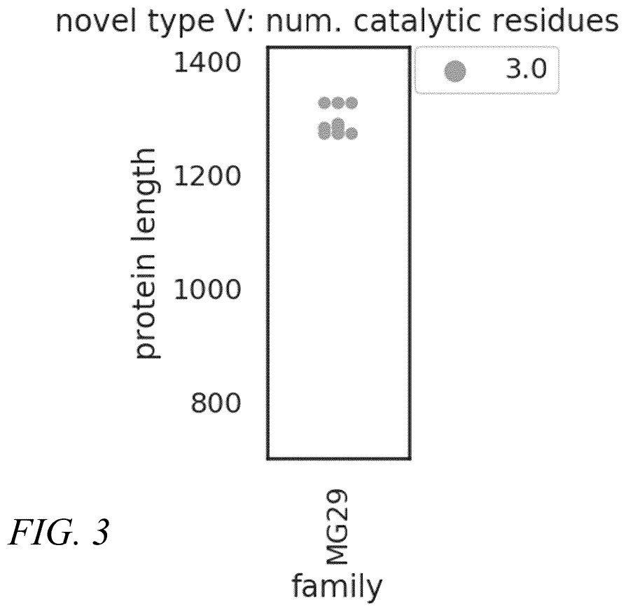

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG11

SEQ ID NOs: 1-37 show the full-length peptide sequences of MG11 nucleases.

SEQ ID NO: 3471 shows a crRNA 5′ direct repeats designed to function with an MG11 nuclease.

SEQ ID NOs: 3472-3538 show effector repeat motifs of MG11 nucleases.

SEQ ID NOs: 38-118 show the full-length peptide sequences of MG13 nucleases.

SEQ ID NO: 3540-3550 show effector repeat motifs of MG13 nucleases.

### MG19

SEQ ID NOs: 119-124 show the full-length peptide sequences of MG19 nucleases.

SEQ ID NOs: 3551-3558 show the nucleotide sequences of sgRNAs engineered to function with a MG19 nuclease.

Sequence Numbers: A3863-A3866 show PAM sequences compatible with MG19 nucleases.

### MG20

SEQ ID NO: 125 shows the full-length peptide sequence of a MG20 nuclease.

SEQ ID NO: 3559 shows the nucleotide sequence of a sgRNA engineered to function with a MG20 nuclease.

Sequence Number: A3867 shows a PAM sequence compatible with an MG20 nuclease.

### MG26

SEQ ID NOs: 126-140 show the full-length peptide sequences of MG26 nucleases.

SEQ ID NOs: 3560-3572 show effector repeat motifs of MG26 nucleases.

### MG28

SEQ ID NOs: 141-214 show the full-length peptide sequences of MG28 nucleases.

SEQ ID NOs: 3573-3607 show effector repeat motifs of MG28 nucleases.

SEQ ID NOs: 3608-3609 show crRNA 5′ direct repeats designed to function with an MG28 nuclease.

Sequence Numbers: A3868-A3869 shows a PAM sequence compatible with an MG28 nuclease.

### MG29

SEQ ID NOs: 215-225 show the full-length peptide sequences of MG29 nucleases.

SEQ ID NO: 5680 shows the nucleotide sequence of an MG29-1 nuclease containing 5′ UTR, NLS, CDS, NLS, 3′ UTR, and polyA tail.

SEQ ID NOs: 3610-3611 show effector repeat motifs of MG29 nucleases.

SEQ ID NO: 3612 shows the nucleotide sequence of a sgRNA engineered to function with a MG29 nuclease.

Sequence Numbers: A3870-A3872 show PAM sequences compatible with an MG29 nuclease.

SEQ ID NO: 5687 shows an MG29-1 coding sequence used for the generation of mRNA.

SEQ ID NOs: 5830 and 5846 show DNA sequences encoding MG29-1 mRNAs.

### MG30

SEQ ID NOs: 226-228 show the full-length peptide sequences of MG30 nucleases.

SEQ ID NOs: 3613-3615 show effector repeat motifs of MG30 nucleases.

Sequence Number: A3873 shows a PAM sequence compatible with an MG30 nuclease.

### MG31

SEQ ID NOs: 229-260 show the full-length peptide sequences of MG31 nucleases.

SEQ ID NOs: 3616-3632 show effector repeat motifs of MG31 nucleases.

Sequence Numbers: A3874-A3876 show PAM sequences compatible with a MG31 nuclease.

### MG32

SEQ ID NO: 261 shows the full-length peptide sequence of a MG32 nuclease.

SEQ ID NO: 3633-3634 show effector repeat motifs of MG32 nucleases.

Sequence Number: A3876 shows a PAM sequence compatible with a MG32 nuclease.

### MG37

SEQ ID NOs: 262-426 show the full-length peptide sequences of MG37 nucleases.

SEQ ID NO: 3635 shows an effector repeat motif of MG37 nucleases.

SEQ ID NOs: 3636-3637, 3640-3641, 3644-3645, 3648-3649, 3652-3653, 3656-3657, and 3660-3661 show the nucleotide sequence of sgRNA engineered to function with an MG37 nuclease.

SEQ ID NOs: 3638, 3642, 3646, 3650, 3654, 3658, and 3662 show the nucleotide sequences of MG37 tracrRNAs derived from the same loci as MG37 nucleases above.

SEQ ID NO: 3639, 3643, 3647, 3651, 3655, and 3659 show 5′ direct repeat sequences derived from native MG37 loci that serve as crRNAs when placed 5′ to a 3′ targeting or spacer sequence.

### MG53

SEQ ID NOs: 427-428 show the full-length peptide sequences of MG53 nucleases.

SEQ ID NO: 3663 shows a 5′ direct repeat sequence derived from native MG53 loci that serve as a crRNA when placed 5′ to a 3′ targeting or spacer sequence.

SEQ ID NOs: 3664-3667 show the nucleotide sequence of sgRNAs engineered to function with an MG53 nuclease.

SEQ ID NOs: 3668-3669 show the nucleotide sequences of MG53 tracrRNAs derived from the same loci as MG53 nucleases above.

### MG54

SEQ ID NOs: 429-430 show the full-length peptide sequences of MG54 nucleases.

SEQ ID NO: 3670 shows a 5′ direct repeat sequence derived from native MG54 loci that serve as a crRNA when placed 5′ to a 3′ targeting or spacer sequence.

SEQ ID NOs: 3671-3672 show the nucleotide sequence of sgRNA engineered to function with an MG54 nuclease.

SEQ ID NOs: 3673-3676 show the nucleotide sequences of MG54 tracrRNAs derived from the same loci as MG54 nucleases above.

### MG55

SEQ ID NOs: 431-688 show the full-length peptide sequences of MG55 nucleases.

SEQ ID NO: 6031 shows the nucleotide sequence of an sgRNA engineered to function with an MG55 nuclease.

Sequence Number: A6032 shows a PAM sequence compatible with an MG55 nuclease.

### MG56

SEQ ID NOs: 689-690 show the full-length peptide sequences of MG56 nucleases.

SEQ ID NO: 3678 shows a crRNA 5′ direct repeats designed to function with an MG56 nuclease.

SEQ ID NOs: 3679-3680 show effector repeat motifs of MG56 nucleases.

### MG57

SEQ ID NOs: 691-721 show the full-length peptide sequences of MG57 nucleases.

SEQ ID NOs: 3681-3694 show effector repeat motifs of MG57 nucleases.

SEQ ID NOs: 3695-3696 show the nucleotide sequences of sgRNAs engineered to function with an MG57 nuclease.

Sequence Numbers: A3879-A3880 shows PAM sequences compatible with MG57 nucleases.

### MG58

SEQ ID NOs: 722-779 show the full-length peptide sequences of MG58 nucleases.

SEQ ID NOs: 3697-3711 show effector repeat motifs of MG58 nucleases.

### MG59

SEQ ID NOs: 780-792 show the full-length peptide sequences of MG59 nucleases.

SEQ ID NOs: 3712-3728 show effector repeat motifs of MG59 nucleases.

SEQ ID NOs: 3729-3730 show the nucleotide sequences of sgRNAs engineered to function with an MG59 nuclease.

Sequence Numbers: A3881-A3882 shows PAM sequences compatible with MG59 nucleases.

### MG60

SEQ ID NOs: 793-1163 show the full-length peptide sequences of MG60 nucleases.

SEQ ID NOs: 3731-3733 show effector repeat motifs of MG60 nucleases.

### MG61

SEQ ID NOs: 1164-1469 show the full-length peptide sequences of MG61 nucleases.

SEQ ID NOs: 3734-3735 show crRNA 5′ direct repeats designed to function with MG61 nucleases.

SEQ ID NOs: 3736-3847 show effector repeat motifs of MG61 nucleases.

### MG62

SEQ ID NOs: 1470-1472 show the full-length peptide sequences of MG62 nucleases.

SEQ ID NOs: 3848-3850 show effector repeat motifs of MG62 nucleases.

### MG70

SEQ ID NOs: 1473-1514 show the full-length peptide sequences of MG70 nucleases.

### MG75

SEQ ID NOs: 1515-1710 show the full-length peptide sequences of MG75 nucleases.

### MG77

SEQ ID NOs: 1711-1712 show the full-length peptide sequences of MG77 nucleases.

SEQ ID NOs: 3851-3852 show the nucleotide sequences of sgRNAs engineered to function with an MG77 nuclease.

Sequence Numbers: A3883-A3884 show PAM sequences compatible with MG77 nucleases.

### MG78

SEQ ID NOs: 1713-1717 show the full-length peptide sequences of MG78 nucleases.

SEQ ID NO: 3853 shows the nucleotide sequence of a sgRNA engineered to function with an MG78 nuclease.

Sequence Number: A3885 shows a PAM sequence compatible with a MG78 nuclease.

### MG79

SEQ ID NOs: 1718-1722 show the full-length peptide sequences of MG79 nucleases.

SEQ ID NOs: 3854-3857 shows the nucleotide sequences of sgRNAs engineered to function with an MG79 nuclease.

Sequence Numbers: A3886-A3889 show the PAM sequences compatible with MG79 nucleases.

### MG80

SEQ ID NO: 1723 shows the full-length peptide sequence of a MG80 nuclease.

### MG81

SEQ ID NOs: 1724-2654 show the full-length peptide sequences of MG81 nucleases.

### MG82

SEQ ID NOs: 2655-2657 show the full-length peptide sequences of MG82 nucleases.

### MG83

SEQ ID NOs: 2658-2659 show the full-length peptide sequences of MG83 nucleases.

### MG84

SEQ ID NOs: 2660-2677 show the full-length peptide sequences of MG84 nucleases.

### MG85

SEQ ID NOs: 2678-2680 show the full-length peptide sequences of MG85 nucleases.

### MG90

SEQ ID NOs: 2681-2809 show the full-length peptide sequences of MG90 nucleases.

### MG91

SEQ ID NOs: 2810-3470 show the full-length peptide sequences of MG91 nucleases.

SEQ ID NOs: 6033-6036 show nucleotide sequences of sgRNAs engineered to function with MG91 nucleases.

Sequence Numbers: A6037-A6039 show PAM sequences compatible with MG91 nucleases.

SEQ ID NOs: 6040-6049 show MG91 intergenic regions potentially encoding tracrRNA.

SEQ ID NOs: 6050-6059 show MG91 CRISPR repeats.

### Spacer Segments

SEQ ID NOs: 3858-3861 show the nucleotide sequences of spacer segments.

### NLS

SEQ ID NOs: 3938-3953 show the sequences of example nuclear localization sequences (NLSs) that can be appended to nucleases according to the disclosure.

### CD38 Targeting

SEQ ID NOs: 4428-4465 and 5685 show the nucleotide sequences of sgRNAs engineered to function with an MG29-1 nuclease in order to target CD38.

SEQ ID NOs: 4466-4503 and 5686 show the DNA sequences of CD38 target sites.

### TIGIT Targeting

SEQ ID NOs: 4504-4520 show the nucleotide sequences of sgRNAs engineered to function with an MG29-1 nuclease in order to target TIGIT.

SEQ ID NOs: 4521-4537 show the DNA sequences of TIGIT target sites.

### AAVS1 Targeting

SEQ ID NOs: 4538-4568 show the nucleotide sequences of sgRNAs engineered to function with an MG29-1 nuclease in order to target AAVS1.

SEQ ID NOs: 4569-4599 show the DNA sequences of AAVS1 target sites.

### B2M Targeting

SEQ ID NOs: 4600-4675 show the nucleotide sequences of sgRNAs engineered to function with an MG29-1 nuclease in order to target B2M.

SEQ ID NOs: 4676-4751 show the DNA sequences of B2M target sites.

### CD2 Targeting

SEQ ID NOs: 4752-4836 show the nucleotide sequences of sgRNAs engineered to function with an MG29-1 nuclease in order to target CD2.

SEQ ID NOs: 4837-4921 show the DNA sequences of CD2 target sites.

### CD5 Targeting

SEQ ID NOs: 4922-4945 show the nucleotide sequences of sgRNAs engineered to function with an MG29-1 nuclease in order to target CD5.

SEQ ID NOs: 4946-4969 show the DNA sequences of CD5 target sites.

hRosa26 Targeting

SEQ ID NOs: 4970-5012 show the nucleotide sequences of sgRNAs engineered to function with an MG29-1 nuclease in order to target hRosa26.

SEQ ID NOs: 5013-5055 show the DNA sequences of hRosa26 target sites.

### TRAC Targeting

SEQ ID NOs: 5056-5125, 5681, and 5683 show the nucleotide sequences of sgRNAs engineered to function with an MG29-1 nuclease in order to target TRAC.

SEQ ID NOs: 5126-5195, 5682, and 5684 show the DNA sequences of TRAC target sites.

### TRBC1 Targeting

SEQ ID NOs: 5196-5210 show the nucleotide sequences of sgRNAs engineered to function with an MG29-1 nuclease in order to target TRBC1.

SEQ ID NOs: 5211-5225 show the DNA sequences of TRBC1 target sites.

### TRBC2 Targeting

SEQ ID NOs: 5226-5246 show the nucleotide sequences of sgRNAs engineered to function with an MG29-1 nuclease in order to target TRBC2.

SEQ ID NOs: 5247-5267 show the DNA sequences of TRBC2 target sites.

### TRBC1/2 Targeting

SEQ ID NOs: 5642-5660 show the nucleotide sequences of sgRNAs engineered to function with an MG29-1 nuclease in order to target TRBC.

SEQ ID NOs: 5661-5679 show the DNA sequences of TRBC target sites.

### FAS Targeting

SEQ ID NOs: 5268-5366 show the nucleotide sequences of sgRNAs engineered to function with an MG29-1 nuclease in order to target FAS.

SEQ ID NOs: 5367-5465 show the DNA sequences of FAS target sites.

### PD-1 Targeting

SEQ ID NOs: 5466-5473 show the nucleotide sequences of sgRNAs engineered to function with an MG29-1 nuclease in order to target PD-1.

SEQ ID NOs: 5474-5481 show the DNA sequences of PD-1 target sites.

### HPRT Targeting

SEQ ID NOs: 5482-5561 show the nucleotide sequences of sgRNAs engineered to function with an MG29-1 nuclease in order to target HPRT.

SEQ ID NOs: 5562-5641 show the DNA sequences of HPRT target sites.

### HAO-1 Targeting

SEQ ID NOs: 5788-5829 and 5831-5834 show the nucleotide sequences of sgRNAs engineered to function with an MG29-1 nuclease in order to target human HAO-1.

SEQ ID NOs: 5836-5845 show the nucleotide sequences of sgRNAs engineered to function with an MG29-1 nuclease in order to target mouse HAO-1.

### APO-A1 Targeting

SEQ ID NOs: 5847-5860 show the nucleotide sequences of sgRNAs engineered to function with an MG29-1 nuclease in order to target mouse APO-A1.

SEQ ID NOs: 5861-5874 show the DNA sequences of APO-A1 target sites.

### ANGPTL3 Targeting

SEQ ID NOs: 5875-5952 show the nucleotide sequences of sgRNAs engineered to function with an MG29-1 nuclease in order to target mouse ANGPTL3.

SEQ ID NOs: 5953-6030 show the DNA sequences of ANGPTL3 target sites.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual'4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R Taylor eds. (1995)), Harlow and Lane, eds. (1988) Antibodies, A Laboratory Manual, and Culture of Animal Cells: A Manual of Basic Technique and Specialized Applications'6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Il.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88 (which is entirely incorporated by reference herein).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic elements to promote transcriptional expression of an operably linked polynucleotide. Eukaryotic basal promoters can contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” can generally be used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

As used herein, the term “Cas12a” generally refers to a family of Cas endonucleases that are class 2, Type V-A Cas endonucleases and that (a) use a relatively small guide RNA (about 42-44 nucleotides) that is processed by the nuclease itself following transcription from the CRISPR array, and (b) cleave DNA to leave staggered cut sites. Further features of this family of enzymes can be found, e.g. in Zetsche B, Heidenreich M, Mohanraju P, et al. Nat Biotechnol 2017; 35:31-34, and Zetsche B, Gootenberg J S, Abudayyeh O O, et al. Cell 2015; 163:759-771, which are incorporated by reference herein.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence” or “spacer sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, generally refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

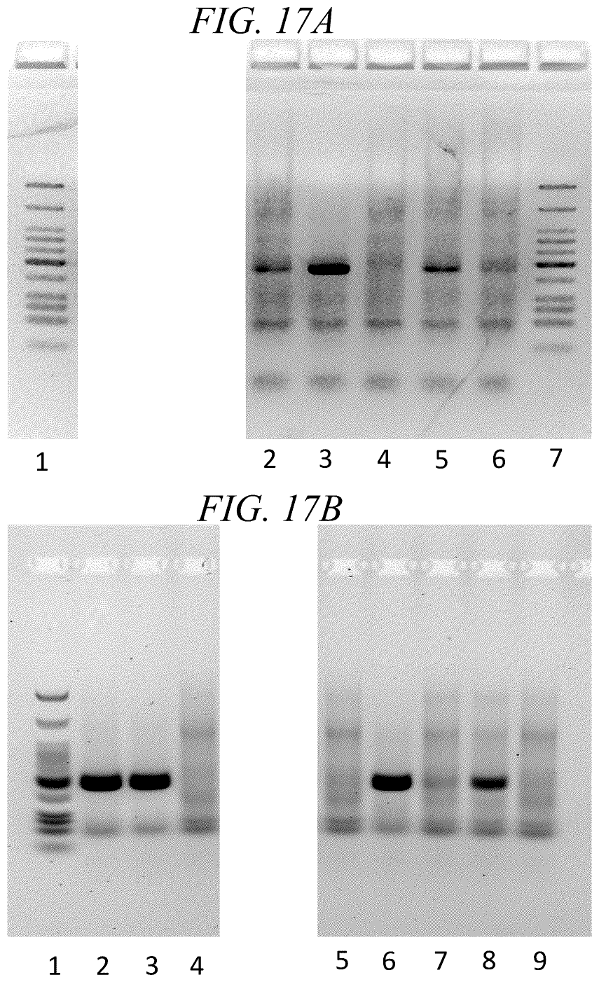

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% sequence identity to any one of the endonuclease protein sequences described herein (e.g. MG11, MG13, MG26, MG28, MG29, MG30, MG31, MG32, MG37, MG53, MG54, MG55, MG56, MG57, MG58, MG59, MG60, MG61, MG62, MG70, MG82, MG83, MG84 or MG85 family endonucleases described herein, or any other family nuclease described herein). In some embodiments, such conservatively substituted variants are functional variants. Such functional variants can encompass sequences with substitutions such that the activity of one or more critical active site residues or guide RNA binding residues of the endonuclease are not disrupted. In some embodiments, a functional variant of any of the proteins described herein lacks substitution of at least one of the conserved or functional residues called out in FIG. 17, 18, 10, 20, or 25 or a residue described in Table 1B. In some embodiments, a functional variant of any of the proteins described herein lacks substitution of all of the conserved or functional residues called out in FIG. 17, 18, 10, 20, or 25 or a residue described in Table 1B.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues identified in Table 1B.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches containing large numbers of microbial species may offer the potential to drastically increase the number of new CRISPR/Cas systems characterized and speed the discovery of new oligonucleotide editing functionalities. A recent example of the fruitfulness of such an approach is demonstrated by the 2016 discovery of CasX/CasY CRISPR systems from metagenomic analysis of natural microbial communities.

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity (see FIG.).

Class I CRISPR-Cas systems have large, multi-subunit effector complexes, and comprise Types I, III, and IV. Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are identified as DNA nucleases. Type 2 effectors generally exhibit a structure comprising a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again identified as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

CRISPR-Cas systems have emerged in recent years as the gene editing technology of choice due to their targetability and ease of use. The most commonly used systems are the Class 2 Type II SpCas9 and the Class 2 Type V-A Cas12a. The Type V-A systems in particular are becoming more widely used since their reported specificity in cells is higher than other nucleases, with fewer or no off-target effects. The V-A systems are also advantageous in that the guide RNA is small (42-44 nucleotides compared with approximately 100 nt for SpCas9) and is processed by the nuclease itself following transcription from the CRISPR array, simplifying multiplexed applications with multiple gene edits. Furthermore, the V-A systems have staggered cut sites, which may facilitate directed repair pathways, such as microhomology-dependent targeted integration (MITI).

The most commonly used Type V-A enzymes require a 5′ protospacer adjacent motif (PAM) next to the chosen target site: 5′-TTV-3′ for Lachnospiraceae bacterium ND2006 LbCas12a and Acidaminococcus sp. AsCas12a; and 5′-TTV-3′ for Francisella novicida FnCas12a. Recent exploration of orthologs has revealed proteins with less restrictive PAM sequences that are also active in mammalian cell culture, for example YTV, YYN or TTN. However, these enzymes do not fully encompass V-A biodiversity and targetability, and may not represent all possible activities and PAM sequence requirements. Here, thousands of genomic fragments were mined from numerous metagenomes for Type V-A nucleases. The diversity of identified V-A enzymes may have been expanded and novel systems may have been developed into highly targetable, compact, and precise gene editing agents.

### MG Enzymes

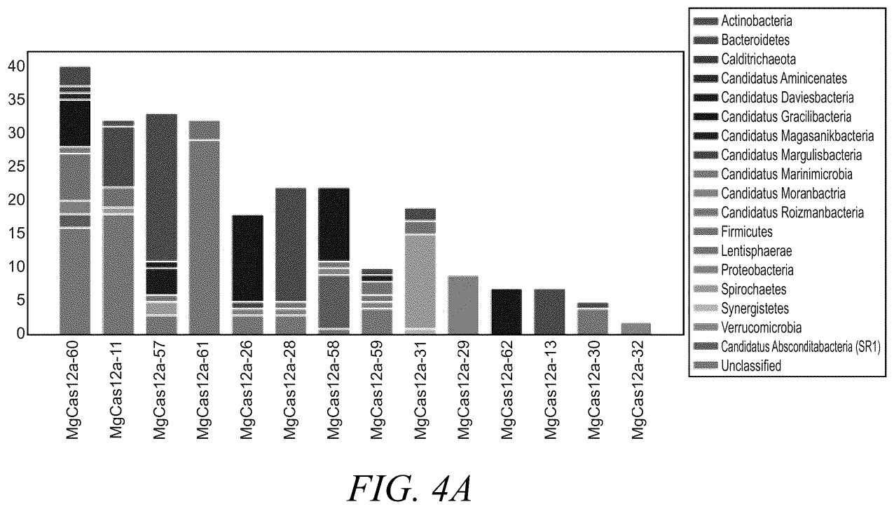

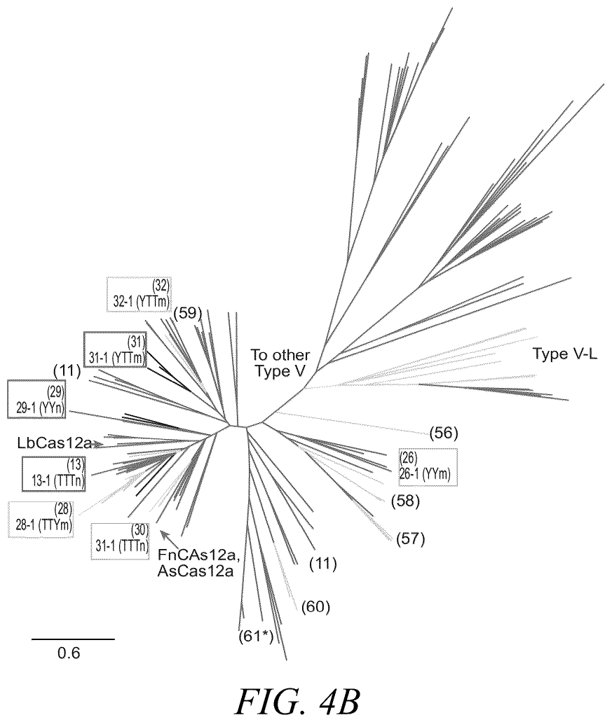

Type V-A CRISPR systems are quickly being adopted for use in a variety of genome editing applications. These programmable nucleases are part of adaptive microbial immune systems, the natural diversity of which has been largely unexplored. Novel families of Type V-A CRISPR enzymes were identified through a large-scale analysis of metagenomes collected from a variety of complex environments, and developed representatives of these systems into gene-editing platforms. The nucleases are phylogenetically diverse (see FIG. 4A) and recognize a single guide RNA with specific motifs. The majority of these systems come from uncultivated organisms, some of which encode a divergent Type V effector within the same CRISPR operon. Biochemical analysis uncovered unexpected PAM diversity (see FIG. 4B), indicating that these systems will facilitate a variety of genome engineering applications. The simplicity of guide sequences and activity in human cell lines suggest utility in gene and cell therapies.

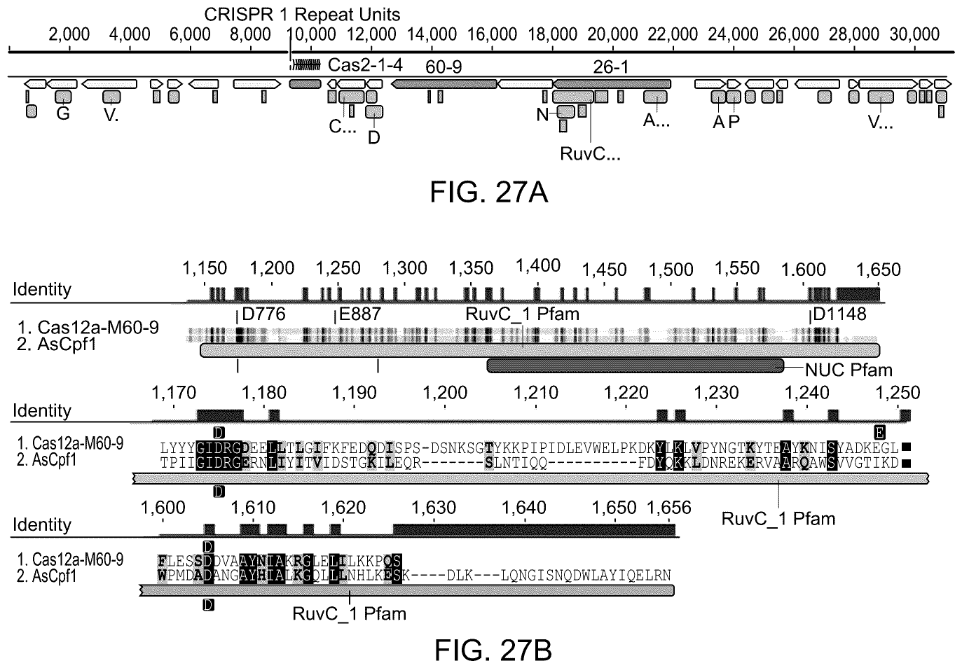

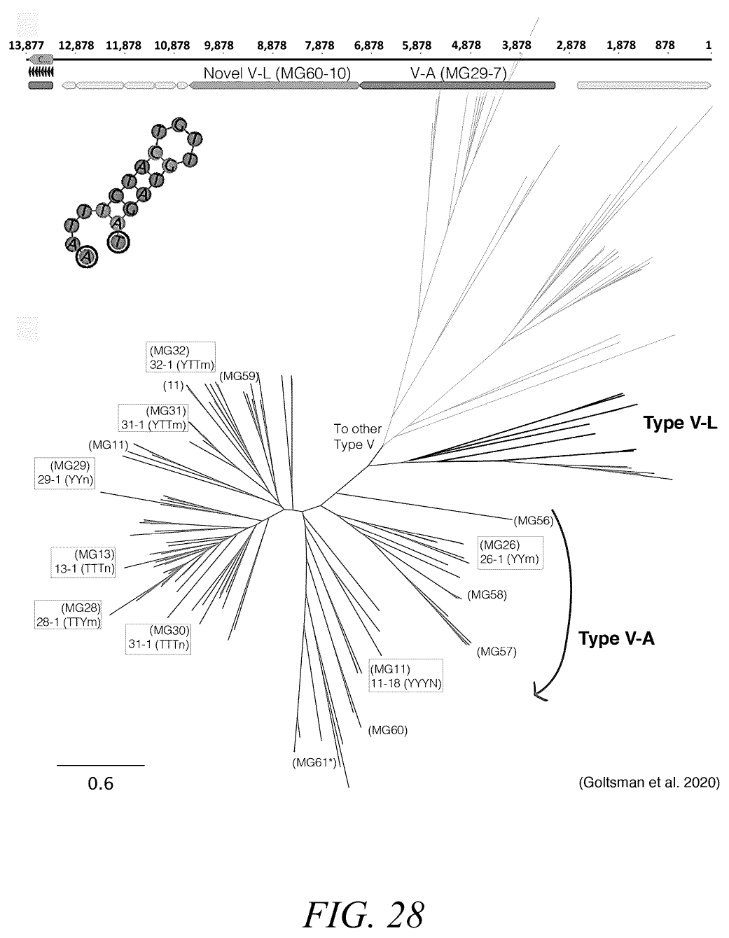

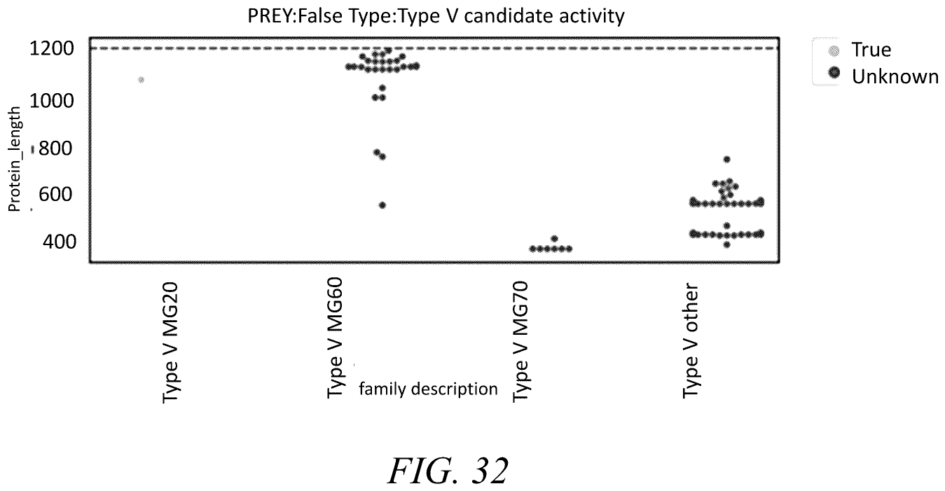

In some aspects, the present disclosure provides for novel Type V-L candidates (see FIGS. 27A-B). Type V-L may be a novel subtype and some sub-families may have been identified. These nucleases are about 1000-1100 amino acids in length. Type V-L may be found in the same CRISPR locus as Type V-A effectors. RuvC catalytic residues may have been identified for Type V-L candidates and these Type V-L candidates may not require tracrRNA. One example of a Type V-L are the MG60 nucleases described herein (see FIG. 28 and FIG. 32).

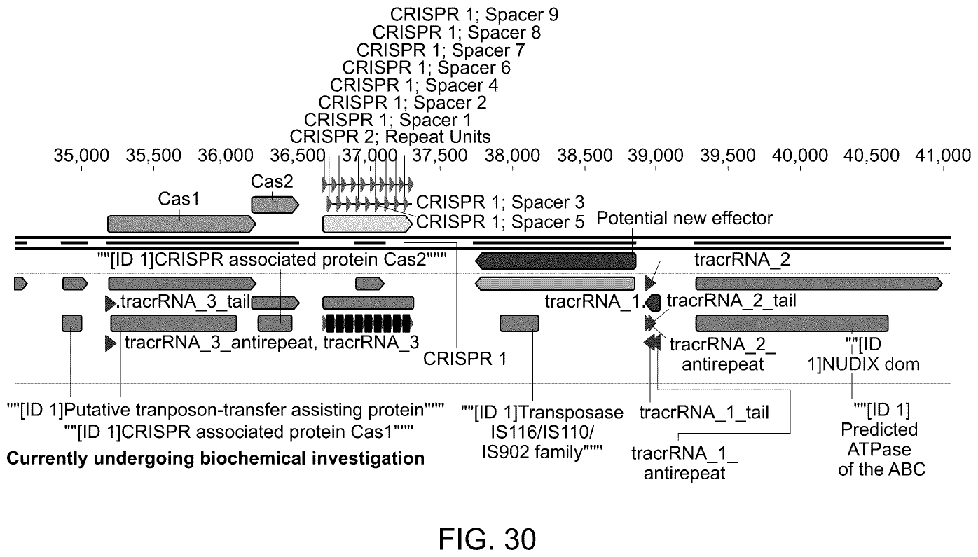

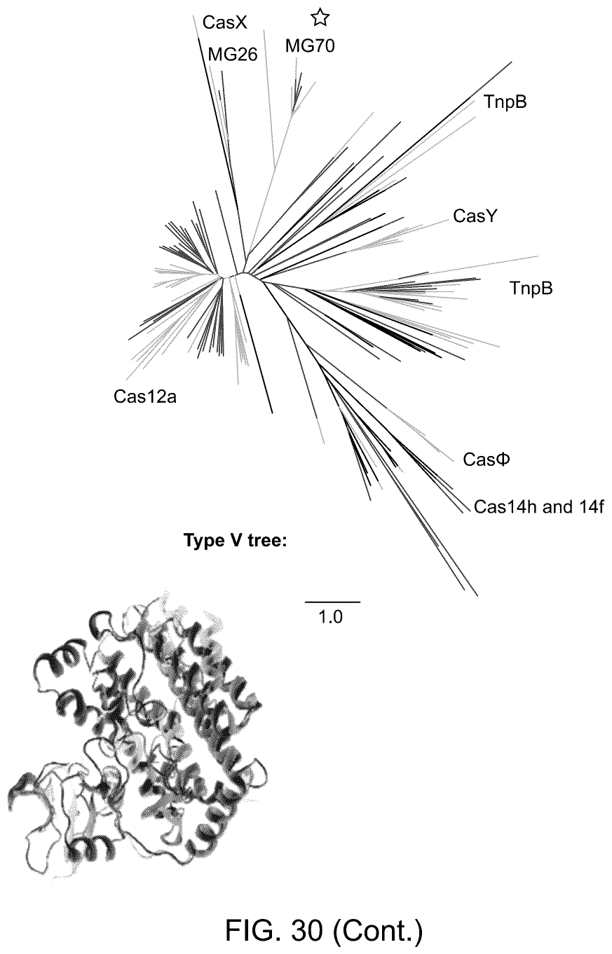

In some aspects, the present disclosure provides for smaller Type V effectors (see FIG. 30). Such effectors may be small putative effectors. These effectors may simplify delivery and may extend therapeutic applications.

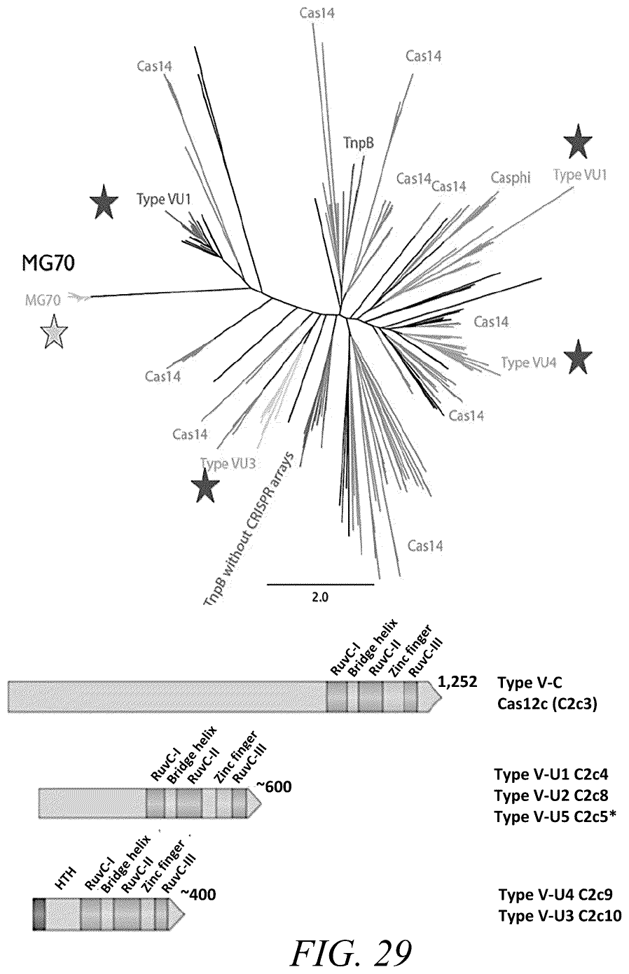

In some aspects, the present disclosure provides for novel type V effector. Such an effector may be MG70 as described herein (see FIG. 29). MG70 may be an ultra-small enzyme of about 373 amino acids in length. MG 70 may have a single transposase domain at the N-terminus and may have a predicted tracrRNA (see FIG. 30 and FIG. 32).

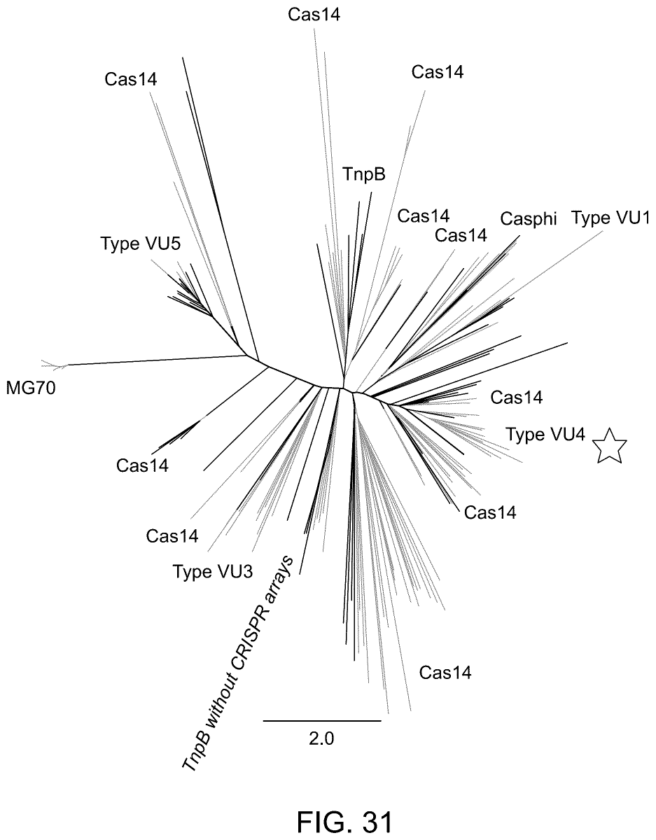

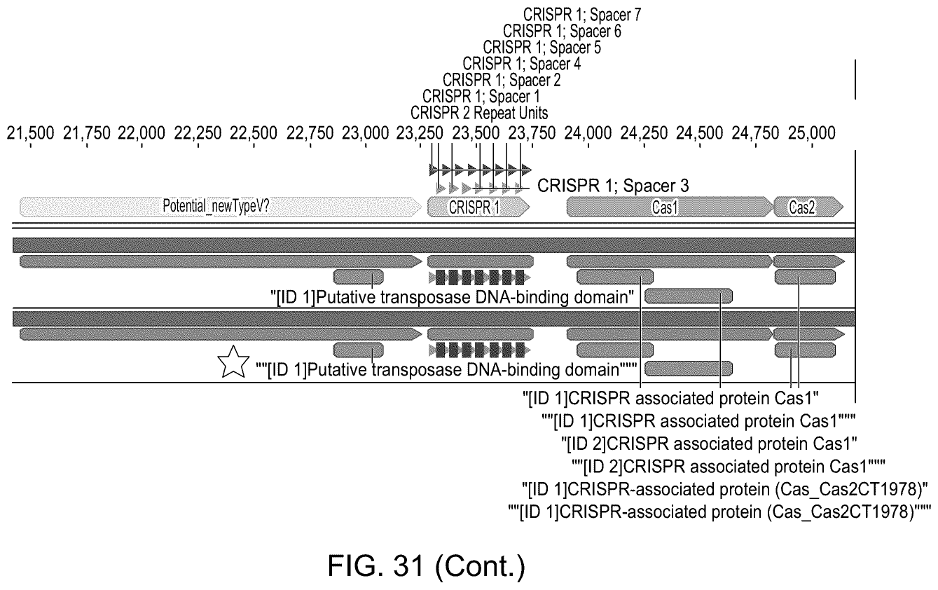

In some aspects, the present disclosure provides for a smaller Type V effector (see FIG. 31). Such an effector may be MG81 described herein. MG81 may be about 500-700 amino acids in length and may contain RuvC, and HTH DNA binding domains.

In one aspect, the present disclosure provides for an engineered nuclease system discovered through metagenomic sequencing. In some cases, the metagenomic sequencing is conducted on samples. In some cases, the samples may be collected from a variety of environments. Such environments may be a human microbiome, an animal microbiome, environments with high temperatures, environments with low temperatures. Such environments may include sediment. An example of the types of such environments of the engineered nuclease systems described herein may be found in FIG.

In one aspect, the present disclosure provides for an engineered nuclease system comprising (a) an endonuclease. In some cases, the endonuclease is a Cas endonuclease. In some cases, the endonuclease is a class 2, type V Cas endonuclease. In some cases, the endonuclease is a class 2, type V-A Cas endonuclease. In some cases, the endonuclease is derived from an uncultivated microorganism. The endonuclease may comprise a RuvC domain. In some cases, the engineered nuclease system comprises (b) an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence.

In one aspect, the present disclosure provides for an engineered nuclease system comprising (a) an endonuclease. In some cases, the endonuclease has at least about 70% sequence identity to any one of SEQ ID NOs: 1-3470. In some cases, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-3470.

In some cases, the endonuclease comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-3470. In some cases, the endonuclease may be substantially identical to any one of SEQ ID NOs: 1-3470.

In some cases, the engineered nuclease system comprises an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence.

In one aspect, the present disclosure provides an engineered nuclease system comprising (a) an endonuclease. In some cases, the endonuclease is configured to bind to a protospacer adjacent motif (PAM) sequence. In some cases, the PAM sequence is substantially identical to any one of Sequence Numbers: A3863-A3889 or any one of SEQ ID Nos: 3890-3913. In some cases, the PAM sequence is any one of Sequence Numbers: A3863-A3889 or any one of SEQ ID NOs: 3890-3913. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class 2, type V-A Cas endonuclease. In some cases, the engineered nuclease system comprises (b) an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence.

In some cases, the endonuclease is not a Cpf1 or Cms1 endonuclease. In some cases, the endonuclease further comprises a zinc finger-like domain.

In some cases, the guide RNA comprises a sequence with at least 80% sequence identity to the first 19 nucleotides or the non-degenerate nucleotides of SEQ ID NO: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, or 3851-3857. In some cases, the guide RNA comprises a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, or 3851-3857. In some cases, the guide RNA comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-36%, 3729-3730, 3734-3735, or 3851-3857. In some cases, the guide RNA comprises a sequence which is substantially identical to the first 19 nucleotides or the non-degenerate nucleotides of SEQ ID NO: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-36%, 3729-3730, 3734-3735, or 3851-3857.

In some cases, the guide RNA comprises a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about %%, at least about 97%, at least about 98%, or at least about 99% identity to the first 19 nucleotides or the non-degenerate nucleotides of SEQ ID NO: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-36%, 3729-3730, 3734-3735, or 3851-3857. In some cases, the endonuclease is configured to bind to the engineered guide RNA. In some cases, the Cas endonuclease is configured to bind to the engineered guide RNA. In some cases, the class 2 Cas endonuclease is configured to bind to the engineered guide RNA. In some cases, the class 2, type V Cas endonuclease is configured to bind to the engineered guide RNA. In some cases, the class 2, type V-A Cas endonuclease is configured to bind to the engineered guide RNA.

In some cases, the endonuclease is configured to bind to a protospacer adjacent motif (PAM) sequence comprising any one of Sequence Numbers: A3863-A3889 or any one of SEQ ID NOs: 3890-3913.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some cases, the guide RNA is 30-250 nucleotides in length. In some cases, the guide RNA is 42-44 nucleotides in length. In some cases, the guide RNA is 42 nucleotides in length. In some cases, the guide RNA is 43 nucleotides in length. In some cases, the guide RNA is 44 nucleotides in length. In some cases, the guide RNA is 85-245 nucleotides in length. In some cases, the guide RNA is more than 90 nucleotides in length. In some cases, the guide RNA is less than 245 nucleotides in length.

In some cases, the endonuclease may comprise a variant having one or more nuclear localization sequences (NLSs). The NLS may be proximal to the N- or C-terminus of the endonuclease. The NLS may be appended N-terminal or C-terminal to any one of SEQ ID NOs: 3938-3953, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3938-3953. In some cases, the NLS may comprise a sequence substantially identical to any one of SEQ ID NOs: 3938-3953.

TABLE 1
Example NLS Sequences that may be used with Cas Effectors according to the
disclosure.

| | | SEQ ID |
|---|---|---|
| Source | NLS amino acid sequence | NO: |
| SV40 | PKKKRKV | 3938 |
| nucleoplasmin | KRPAATKKAGQAKKKK | 3939 |
| bipartite NLS | | |
| c-myc NLS | PAAKRVKLD | 3940 |
| c-myc NLS | RQRRNELKRSP | 3941 |
| hRNPA1 M9 NLS | NOSSNFGPMKGGNFGGRSSGPYGGGGQYFAKPRNQGGY | 3942 |
| Importin-alpha IBB | RMRIZFKNKGKDTAELRRRRVEVSVELRKAKKDEQILKRRNV | 3943 |
| domain | | |
| Myoma T protein | VSRKRPRP | 3944 |
| Myoma T protein | PPKKARED | 3945 |
| p53 | PQPKKKPL | 3946 |
| mouse c-abl IV | SALIKKKKKMAP | 3947 |
| influenza virus NS1 | DRLRR | 3948 |
| influenza virus NS1 | PKQKKRK | 3949 |
| Hepatitis virus delta | RKLKKKIKKL | 3950 |
| antigen | | |
| mouse Mx1 protein | REKKKELKRR | 3951 |
| human poly (ADP- | KRKGDEVDGVDEVAKKKSKK | 3952 |
| ribose) polymerase | | |
| steroid hormone | RKCLQAGMNLEARKTKK | 3953 |
| receptors (human) | | |
| glucocorticoid | | |

In some cases, the engineered nuclease system further comprises a single- or double stranded DNA repair template. In some cases, the engineered nuclease system further comprises a single-stranded DNA repair template. In some cases, the engineered nuclease system further comprises a double-stranded DNA repair template. In some cases, the single- or double-stranded DNA repair template may comprise from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence.

In some cases, the first homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides. In some cases, the second homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides.

In some cases, the first and second homology arms are homologous to a genomic sequence of a prokaryote. In some cases, the first and second homology arms are homologous to a genomic sequence of a bacteria. In some cases, the first and second homology arms are homologous to a genomic sequence of a fungus. In some cases, the first and second homology arms are homologous to a genomic sequence of a eukaryote.

In some cases, the engineered nuclease system further comprises a DNA repair template. The DNA repair template may comprise a double-stranded DNA segment. The double-stranded DNA segment may be flanked by one single-stranded DNA segment. The double-stranded DNA segment may be flanked by two single-stranded DNA segments. In some cases, the single-stranded DNA segments are conjugated to the 5′ ends of the double-stranded DNA segment. In some cases, the single stranded DNA segments are conjugated to the 3′ ends of the double-stranded DNA segment.

In some cases, the single-stranded DNA segments have a length from 1 to 15 nucleotide bases. In some cases, the single-stranded DNA segments have a length from 4 to 10 nucleotide bases. In some cases, the single-stranded DNA segments have a length of 4 nucleotide bases. In some cases, the single-stranded DNA segments have a length of 5 nucleotide bases. In some cases, the single-stranded DNA segments have a length of 6 nucleotide bases. In some cases, the single-stranded DNA segments have a length of 7 nucleotide bases. In some cases, the single-stranded DNA segments have a length of 8 nucleotide bases. In some cases, the single-stranded DNA segments have a length of 9 nucleotide bases. In some cases, the single-stranded DNA segments have a length of 10 nucleotide bases.

In some cases, the single-stranded DNA segments have a nucleotide sequence complementary to a sequence within the spacer sequence. In some cases, the double-stranded DNA sequence comprises a barcode, an open reading frame, an enhancer, a promoter, a protein-coding sequence, a miRNA coding sequence, an RNA coding sequence, or a transgene.

In some cases, the engineered nuclease system further comprises a source of Mg²⁺.

In some cases, the guide RNA comprises a hairpin comprising at least 8 base-paired ribonucleotides. In some cases, the guide RNA comprises a hairpin comprising at least 9 base-paired ribonucleotides. In some cases, the guide RNA comprises a hairpin comprising at least 10 base-paired ribonucleotides. In some cases, the guide RNA comprises a hairpin comprising at least 11 base-paired ribonucleotides. In some cases, the guide RNA comprises a hairpin comprising at least 12 base-paired ribonucleotides.

In some cases, the endonuclease comprises a sequence at least 70% identical to a variant of any one of SEQ ID NOs: 141, 215, 229, 261, or 1711-1721 or a variant thereof 141, 215, 229, 261, or 1711-1721 or a variant thereof. In some cases, the endonuclease comprises a sequence at least 75% identical to a variant of any one of SEQ ID NOs: 141, 215, 229, 261, or 1711-1721 or a variant thereof. In some cases, the endonuclease comprises a sequence at least 80% identical to a variant of any one of SEQ ID NOs: 141, 215, 229, 261, or 1711-1721 or a variant thereof. In some cases, the endonuclease comprises a sequence at least 85% identical to a variant of any one of SEQ ID NOs: 141, 215, 229, 261, or 1711-1721 or a variant thereof. In some cases, the endonuclease comprises a sequence at least 90% identical to a variant of any one of SEQ ID NOs: 141, 215, 229, 261, or 1711-1721 or a variant thereof. In some cases, the endonuclease comprises a sequence at least 95% identical to a variant of any one of SEQ ID NOs: 141, 215, 229, 261, or 1711-1721 or a variant thereof.

In some cases, the guide RNA structure comprises a sequence of at least 70% identical to the first 19 nucleotides or the non-degenerate nucleotides of SEQ ID NO: 3608. In some cases, the guide RNA structure comprises a sequence of at least 75% identical to the first 19 nucleotides or the non-degenerate nucleotides of SEQ ID NO: 3608. In some cases, the guide RNA structure comprises a sequence of at least 80% identical to the first 19 nucleotides or the non-degenerate nucleotides of SEQ ID NO: 3608. In some cases, the guide RNA structure comprises a sequence of at least 85% identical to the first 19 nucleotides or the non-degenerate nucleotides of SEQ ID NO: 3608. In some cases, the guide RNA structure comprises a sequence of at least 90% identical to the first 19 nucleotides or the non-degenerate nucleotides of SEQ ID NO: 3608. In some cases, the guide RNA structure comprises a sequence of at least 95% identical to the first 19 nucleotides or the non-degenerate nucleotides of SEQ ID NO: 3608. In some cases, the endonuclease is configured to bind to a PAM comprising any one of Sequence Numbers: A3863-A3889 or any one of SEQ ID NOs: 3890-3913.

In some cases, sequence may be determined by a BLASTP, CLUSTALW, MUSCLE, or MAFFT algorithm, or a CLUSTALW algorithm with the Smith-Waterman homology search algorithm parameters. The sequence identity may be determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In one aspect, the present disclosure provides an engineered guide RNA comprising (a) a DNA-targeting segment. In some cases, the DNA-targeting segment comprises a nucleotide sequence that is complementary to a target sequence. In some cases, the target sequence is in a target DNA molecule. In some cases, the engineered guide RNA comprises (b) a protein-binding segment. In some cases, the protein-binding segment comprises two complementary stretches of nucleotides. In some cases, the two complementary stretches of nucleotides hybridize to form a double-stranded RNA (dsRNA) duplex. In some cases, the two complementary stretches of nucleotides are covalently linked to one another with intervening nucleotides. In some cases, the engineered guide ribonucleic acid polynucleotide is capable of forming a complex with an endonuclease. In some cases, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-3470. In some cases, the complex targets the target sequence of the target DNA molecule.

In some cases, the DNA-targeting segment is positioned 3′ of both of the two complementary stretches of nucleotides. In some cases, the protein binding segment comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 3608.

In some cases, the double-stranded RNA (dsRNA) duplex comprises at least 8 ribonucleotides. In some cases, the double-stranded RNA (dsRNA) duplex comprises at least 9 ribonucleotides. In some cases, the double-stranded RNA (dsRNA) duplex comprises at least 10 ribonucleotides. In some cases, the double-stranded RNA (dsRNA) duplex comprises at least 11 ribonucleotides. In some cases, the double-stranded RNA (dsRNA) duplex comprises at least 12 ribonucleotides.

In some cases, the deoxyribonucleic acid polynucleotide encodes the engineered guide ribonucleic acid polynucleotide.

In one aspect, the present disclosure provides a nucleic acid comprising an engineered nucleic acid sequence. In some cases, the engineered nucleic acid sequence is optimized for expression in an organism. In some cases, the nucleic acid encodes an endonuclease. In some cases, the endonuclease is a Cas endonuclease. In some cases, the endonuclease is a class 2 endonuclease. In some cases, the endonuclease is a class2, type V Cas endonuclease. In some cases, the endonuclease is a class2, type V-A Cas endonuclease. In some cases, the endonuclease is derived from an uncultivated microorganism. In some cases, the organism is not the uncultivated organism.

In some cases, the endonuclease comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-3470.

In some cases, the endonuclease may comprise a variant having one or more nuclear localization sequences (NLSs). The NLS may be proximal to the N- or C-terminus of the endonuclease. The NLS may be appended N-terminal or C-terminal to any one of SEQ ID NOs: 3938-3953, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 3938-3953.

In some cases, the organism is prokaryotic. In some cases, the organism is bacterial. In some cases, the organism is eukaryotic. In some cases, the organism is fungal. In some cases, the organism is a plant. In some cases, the organism is mammalian. In some cases, the organism is a rodent. In some cases, the organism is human.

In one aspect, the present disclosure provides an engineered vector. In some cases, the engineered vector comprises a nucleic acid sequence encoding an endonuclease. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class2, type V-A Cas endonuclease. In some cases, the endonuclease is derived from an uncultivated microorganism.

In some cases, the engineered vector comprises a nucleic acid described herein. In some cases, the nucleic acid described herein is a deoxyribonucleic acid polynucleotide described herein. In some cases, the vector is a plasmid, a minicircle, a CELiD, an adeno-associated virus (AAV) derived virion, or a lentivirus.

In one aspect, the present disclosure provides a cell comprising a vector described herein.

In one aspect, the present disclosure provides a method of manufacturing an endonuclease. In some cases, the method comprises cultivating the cell.

In one aspect, the present disclosure provides a method for binding, cleaving, marking, or modifying a double-stranded deoxyribonucleic acid polynucleotide. The method may comprise contacting the double-stranded deoxyribonucleic acid polynucleotide with an endonuclease. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class2, type V-A Cas endonuclease. In some cases, the endonuclease is in complex with an engineered guide RNA. In some cases, the engineered guide RNA is configured to bind to the endonuclease. In some cases, the engineered guide RNA is configured to bind to the double-stranded deoxyribonucleic acid polynucleotide. In some cases, the engineered guide RNA is configured to bind to the endonuclease and to the double-stranded deoxyribonucleic acid polynucleotide. In some cases, the double-stranded deoxyribonucleic acid polynucleotide comprises a protospacer adjacent motif (PAM). In some cases, the PAM comprises a sequence comprising any one of Sequence Numbers: A3863-A3889 or any one of SEQ ID NOs: 3890-3913.

In some cases, the double-stranded deoxyribonucleic acid polynucleotide comprises a first strand comprising a sequence complementary to a sequence of the engineered guide RNA and a second strand comprising the PAM. In some cases, the PAM is directly adjacent to the 5′ end of the sequence complementary to the sequence of the engineered guide RNA. In some cases, the endonuclease is not a Cpf1 endonuclease or a Cms1 endonuclease. In some cases, the endonuclease is derived from an uncultivated microorganism. In some cases, the double-stranded deoxyribonucleic acid polynucleotide is a eukaryotic, plant, fungal, mammalian, rodent, or human double-stranded deoxyribonucleic acid polynucleotide. In some cases, the PAM comprises any one of Sequence Numbers: A3863-A3889 or any one of SEQ ID NOs: 3890-3913.

In one aspect, the present disclosure provides a method of modifying a target nucleic acid locus. The method may comprise delivering to the target nucleic acid locus the engineered nuclease system described herein. In some cases, the endonuclease is configured to form a complex with the engineered guide ribonucleic acid structure. In some cases, the complex is configured such that upon binding of the complex to the target nucleic acid locus, the complex modifies the target nucleic acid locus.

In some cases, modifying the target nucleic acid locus comprises binding, nicking, cleaving, or marking said target nucleic acid locus. In some cases, the target nucleic acid locus comprises deoxyribonucleic acid (DNA) or ribonucleic acid (RNA). In some cases, the target nucleic acid comprises genomic DNA, viral DNA, viral RNA, or bacterial DNA. In some cases, the target nucleic acid locus is in vitro. In some cases, the target nucleic acid locus is within a cell. In some cases, the cell is a prokaryotic cell, a bacterial cell, a eukaryotic cell, a fungal cell, a plant cell, an animal cell, a mammalian cell, a rodent cell, a primate cell, or a human cell.

In some cases, delivery of the engineered nuclease system to the target nucleic acid locus comprises delivering the nucleic acid described herein or the vector described herein. In some cases, delivery of engineered nuclease system to the target nucleic acid locus comprises delivering a nucleic acid comprising an open reading frame encoding the endonuclease. In some cases, the nucleic acid comprises a promoter. In some cases, the open reading frame encoding the endonuclease is operably linked to the promoter.

In some cases, delivery of the engineered nuclease system to the target nucleic acid locus comprises delivering a capped mRNA containing the open reading frame encoding the endonuclease. In some cases, delivery of the engineered nuclease system to the target nucleic acid locus comprises delivering a translated polypeptide. In some cases, delivery of the engineered nuclease system to the target nucleic acid locus comprises delivering a deoxyribonucleic acid (DNA) encoding the engineered guide RNA operably linked to a ribonucleic acid (RNA) pol III promoter.

In some cases, the endonuclease induces a single-stranded break or a double-stranded break at or proximal to the target locus. In some cases, the endonuclease induces a staggered single stranded break within or 3′ to said target locus.

In some cases, effector repeat motifs are used to inform guide design of MG nucleases. For example, the processed gRNA in Type V-A systems comprises the last 20-22 nucleotides of a CRISPR repeat. This sequence may be synthesized into a crRNA (along with a spacer) and tested in vitro, along with the synthesized nucleases, for cleavage on a library of possible targets. Using this method, the PAM may be determined. In some cases, Type V-A enzymes may use a “universal” gRNA. In some cases, Type V enzymes may utilize a unique gRNA.

### Lipid Nanoparticles

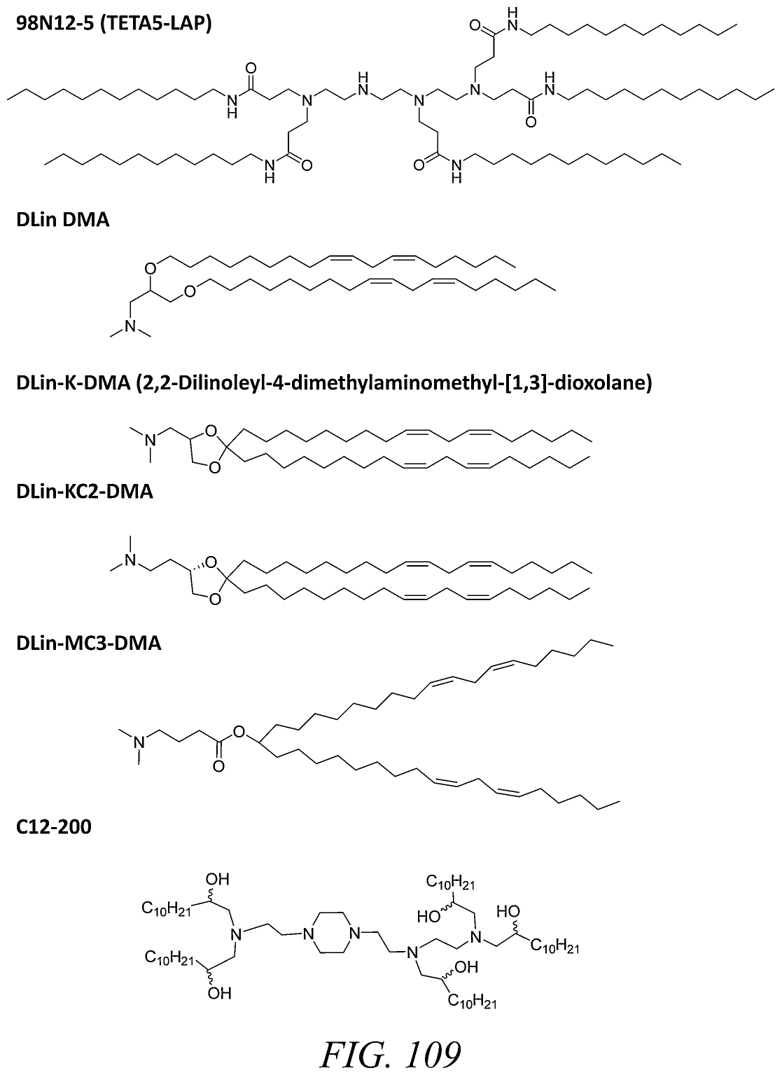

Lipid nanoparticles as described herein can be 4-component lipid nanoparticles. Such nanoparticles can be configured for delivery of RNA or other nucleic acids (e.g. synthetic RNA, mRNA, or in vitro-synthesized mRNA) and can be generally formulated as described in WO2012135805A2, which is incorporated by reference herein for all purposes. Such nanoparticles can generally comprise: (a) a cationic lipid (e.g. any of the lipids described in FIG. 109), (b) a neutral lipid (e.g. DSPC or DOPE), (c) a sterol (e.g. cholesterol or a cholesterol analog), and (d) a PEG-modified lipid (e.g. PEG-DMG).

The cationic lipid referred to herein as “C12-200” is disclosed by Love et al., Proc Natl Acad Sci USA. 2010 107:1864-1869 and Liu and Huang, Molecular Therapy. 2010 669-670; both of which are herein incorporated by reference in their entirety. Cationic lipid formulations can include particles comprising either 3 or 4 or more components in addition to polynucleotide, primary construct, or RNA (e.g. mRNA). As an example, formulations with certain cationic lipids include, but are not limited to, 98N12-5 (or any of the other structures described in FIG. 109) and may contain 42% lipidoid, 48% cholesterol, and 10% PEG (C14 or greater alkyl chain length). As another example, formulations with certain lipidoids include, but are not limited to, C12-200 and may contain 50% cationic lipid, 10% disteroylphosphatidyl choline, 38.5% cholesterol, and 1.5% PEG-DMG.

In some embodiments, lipid nanoparticles are formulated as described in U.S. Ser. No. 10/709,779B2, which is incorporated in its entirety by reference herein. In some embodiments, the cationic lipid nanoparticle comprises a cationic lipid, a PEG-modified lipid, a sterol, and a non-cationic lipid. In some embodiments, the cationic lipid is selected from the group consisting of any of the cationic lipids depicted in FIG. 109. In some embodiments, the cationic lipid nanoparticle has a molar ratio of about 20-60% cationic lipid, about 5-25% non-cationic lipid, about 25-55% sterol, and about 0.5-15% PEG-modified lipid. In some embodiments, the cationic lipid nanoparticle comprises a molar ratio of about 50% cationic lipid, about 1.5% PEG-modified lipid, about 38.5% cholesterol, and about 10% non-cationic lipid. In some embodiments, the cationic lipid nanoparticle comprises a molar ratio of about 55% cationic lipid, about 2.5% PEG-modified lipid, about 32.5% cholesterol, and about 10% non-cationic lipid. In some embodiments, the cationic lipid is an ionizable cationic lipid, the non-cationic lipid is a neutral lipid, and the sterol is a cholesterol. In some embodiments, the cationic lipid nanoparticle has a molar ratio of 50:38.5:10:1.5 of cationic lipid: cholesterol: PEG2000-DMG:DSPC or DMG:DOPE. In some embodiments, lipid nanoparticles as described herein can comprise cholesterol, 1,2-dioleoyl-sn-glycero-3-phosphoethanolamine (DOPE), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), and DMG-PEG-2000 at molar ratios of 47.5:16:35:1.5.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

### Examples

### Example 1—A Method of Metagenomic Analysis for New Proteins

Metagenomic samples were collected from sediment, soil, and animals. Deoxyribonucleic acid (DNA) was extracted with a Zymobiomics DNA mini-prep kit and sequenced on an Illumina HiSeq® 2500. Samples were collected with consent of property owners. Metagenomic sequence data was searched using Hidden Markov Models generated based on identified Cas protein sequences including class II type V Cas effector proteins to identify new Cas effectors (see FIG., which shows distribution of proteins detected in one family, MG29, identified from sample types such as high-temperature samples). Novel effector proteins identified by the search were aligned to identified proteins to identify potential active sites (see e.g. FIG., which shows that all MG29 family effectors identified from various samples have three catalytic residues from RuvCI, RuvCII, and RuvCIII catalytic domains and are predicted to be active). This metagenomic workflow resulted in the delineation of the MG11, MG13, MG19, MG20, MG26, MG28, MG29, MG30, MG31, MG32, MG37, MG53, MG54, MG55, MG56, MG57, MG58, MG59, MG60, MG61, MG62, MG70, MG75, MG77, MG78, MG79, MG80, MG81, MG82, MG83, MG84, MG85, MG90, and MG91 families described herein. Putative spacer sequences were identified by their location adjacent to the genomic loci encoding the effector proteins.

### Example 2—A Method of Metagenomic Analysis for New Proteins

Thirteen animal microbiome, high temperature biofilm and sediment samples were collected and stored on ice or in Zymo DNA/RNA Shield after collection. DNA was extracted from samples using either the Qiagen DNeasy PowerSoil Kit or the ZymoBIOMICS DNA Miniprep Kit. DNA sequencing libraries were constructed and sequenced on an Illumina HiSeq 4000 or on a Novaseq machine at the Vincent J. Coates Genomics Sequencing Laboratory at UC Berkeley, with paired 150 bp reads with a 400-800 bp target insert size (10 GB of sequencing was targeted per sample). Publicly available metagenomic sequencing data were downloaded from the NCBI SRA. Sequencing reads were trimmed using BBMap (Bushnell B., sourceforge.net/projects/bbmap/) and assembled with Megahit 11. Open reading frames and protein sequences were predicted with Prodigal. HMM profiles of identified Type V-A CRISPR nucleases were built and searched against all predicted proteins using HMMER3 (hmmer.org) to identify potential effectors. CRISPR arrays on assembled contigs were predicted with Minced (https://github.com/ctSkennerton/minced). Taxonomy was assigned to proteins with Kaiju, and contig taxonomy was determined by finding the consensus of all encoded proteins.

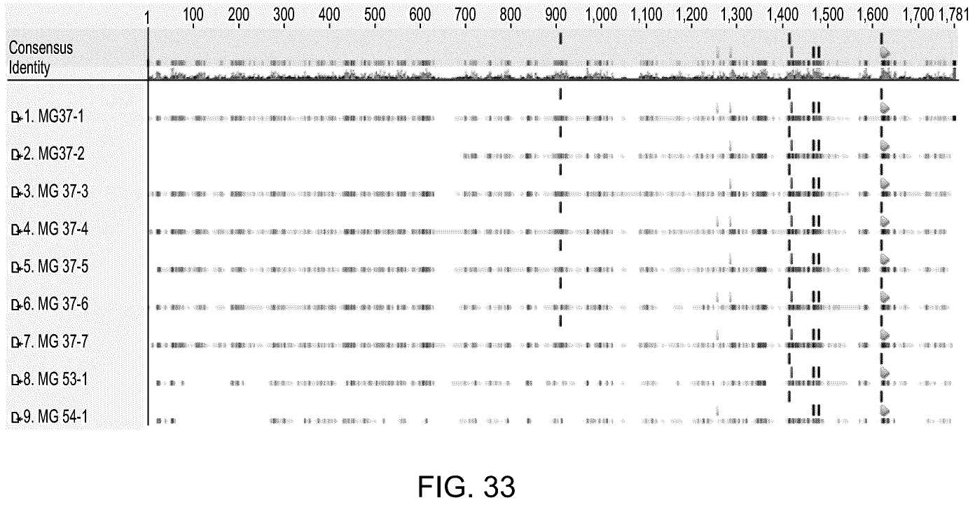

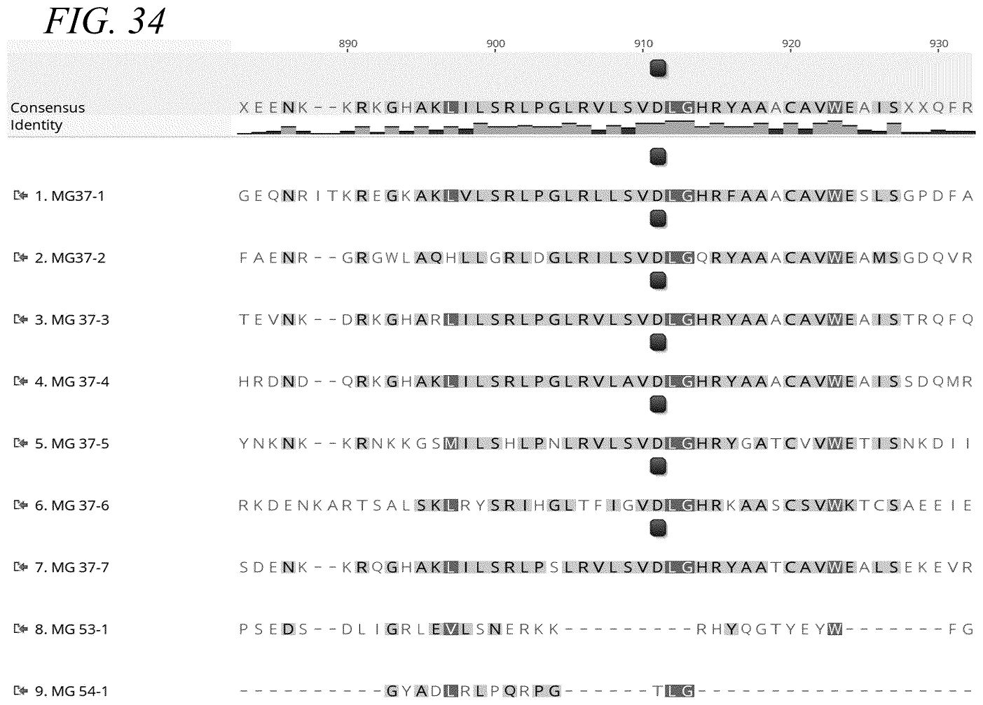

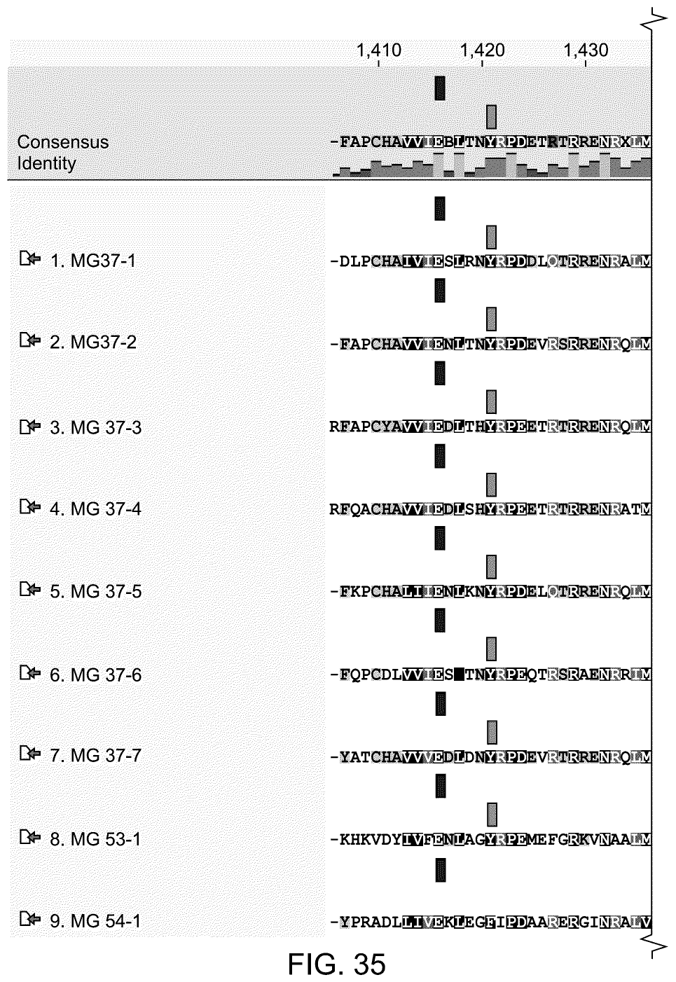

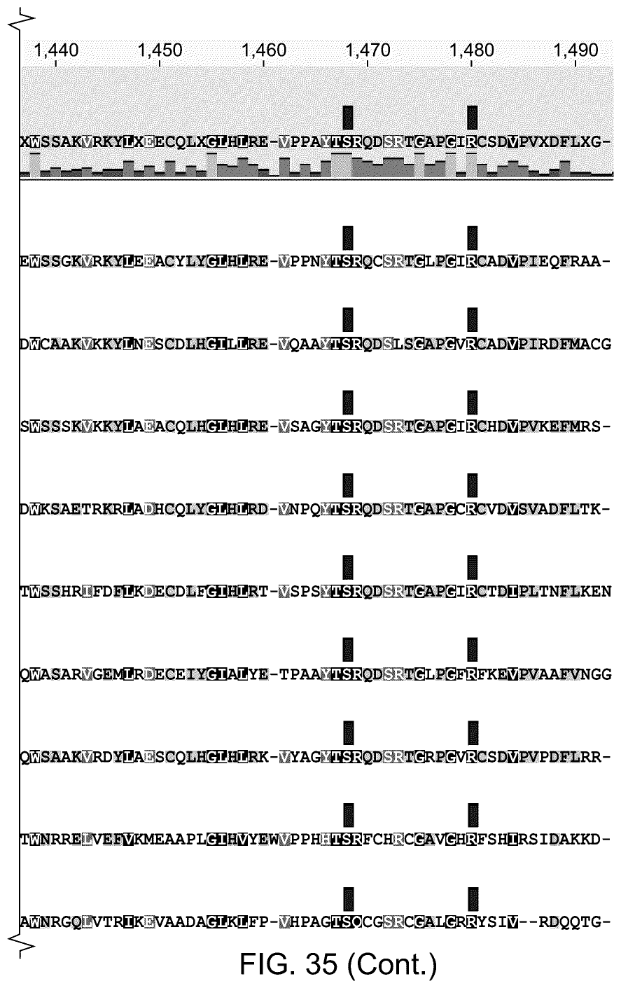

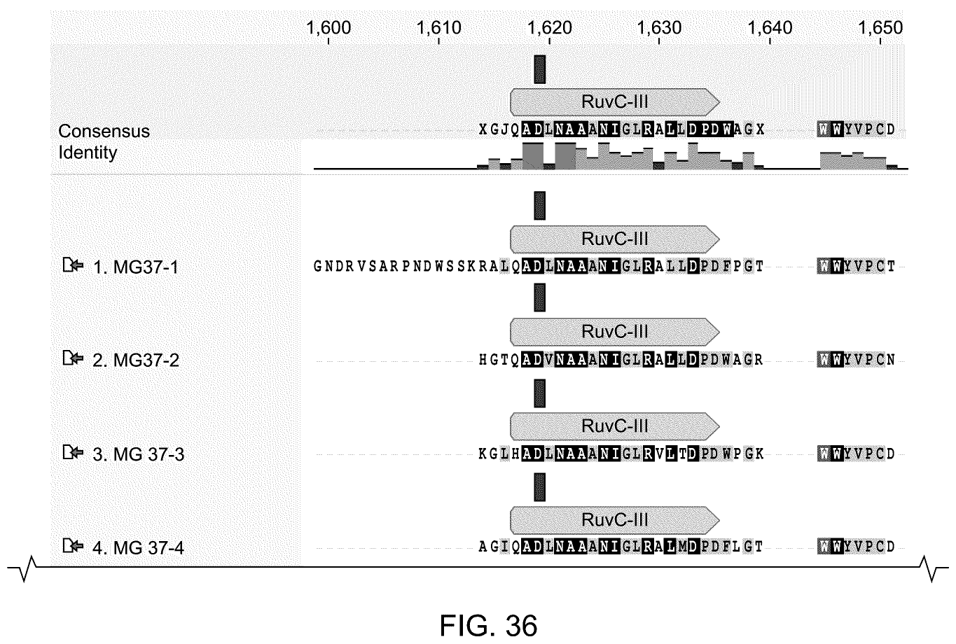

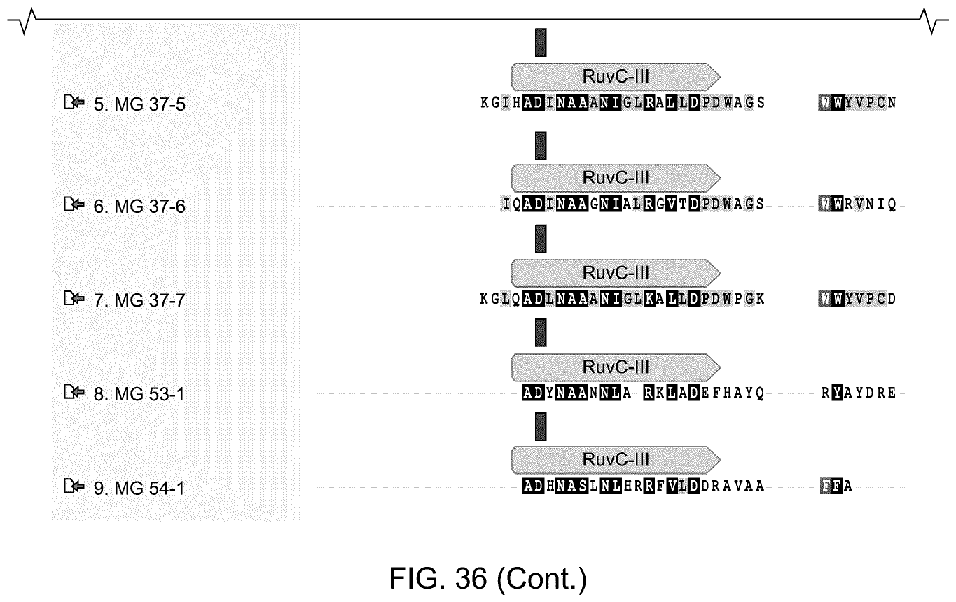

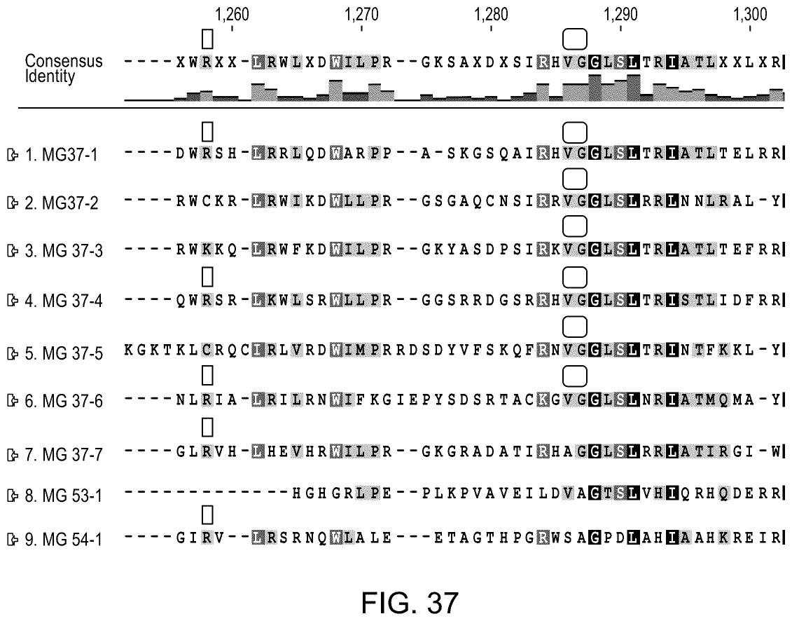

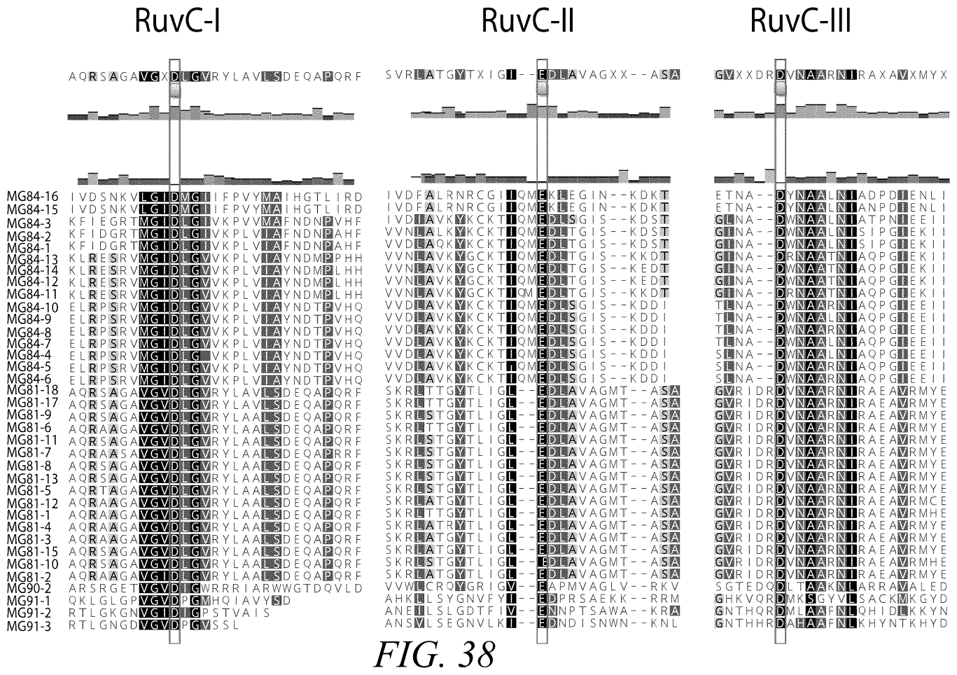

Predicted and reference (e.g., LbCas12a, AsCas12a, FnCas12a) Type V effector proteins were aligned with MAFFT and a phylogenetic tree was inferred using FasTree2. Novel families were delineated by identifying clades composed of sequences recovered from this study. From within families, candidates were selected if they contained the components for laboratory analysis (i.e., they were found on a well-assembled and annotated contig with a CRISPR array) in a manner that sampled as much phylogenetic diversity as possible. Priority was given to small effectors from diverse families (that is, families with representatives sharing a wider range of protein sequences). Selected representative and reference sequences were aligned using MUSCLE and Clustal W to identify catalytic and PAM interacting residues. CRISPR array repeats were searched for a motif associated with Type V-A systems, TCTAC-N-GTAGA (containing between one and eight N residues). From this analysis, families were putatively classified as V-A if representative CRISPR arrays contained one of these motif sequences. This dataset was used to identify HMM profiles associated with V-A families, which were in turn used to classify additional families (see FIG. 33-FIG. 37). Although the convention is to name novel Cas12 nucleases on the basis of the organism that encodes them, it is not possible to do so for the nucleases described herein. Therefore, in order to best adhere to the convention, the systems described herein are named with the prefix MG to indicate they are derived from assembled metagenomic fragments.

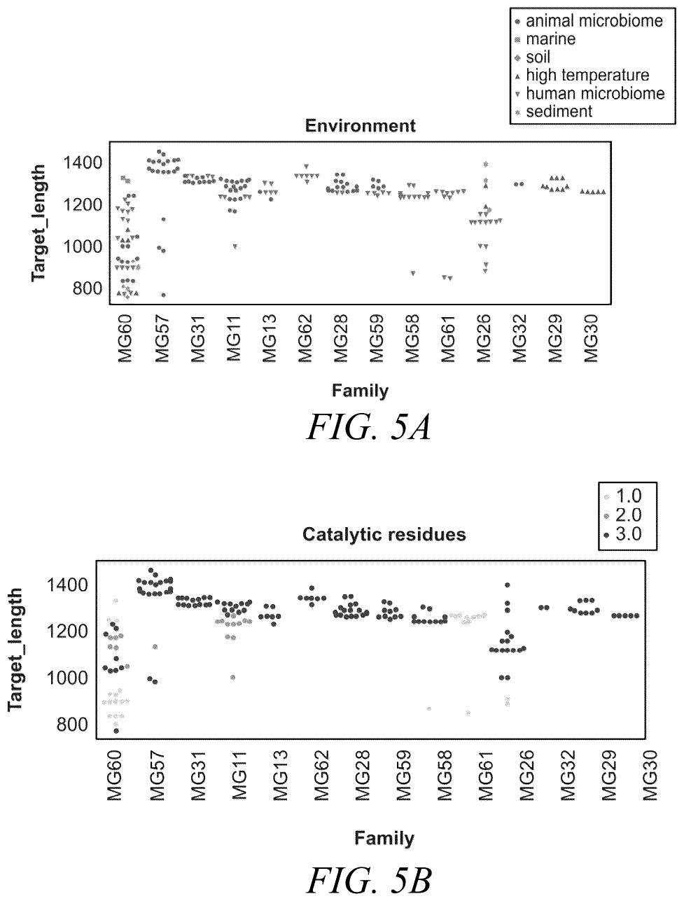

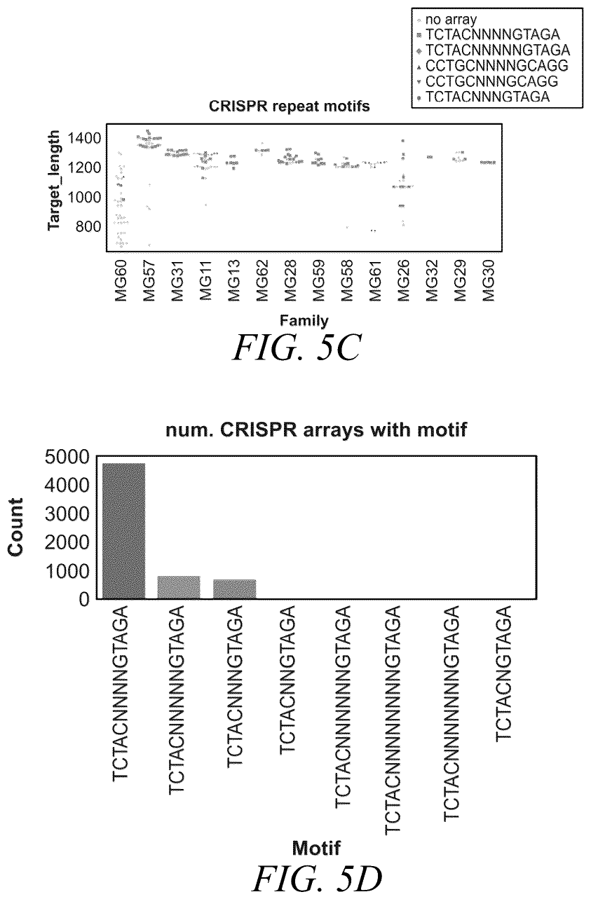

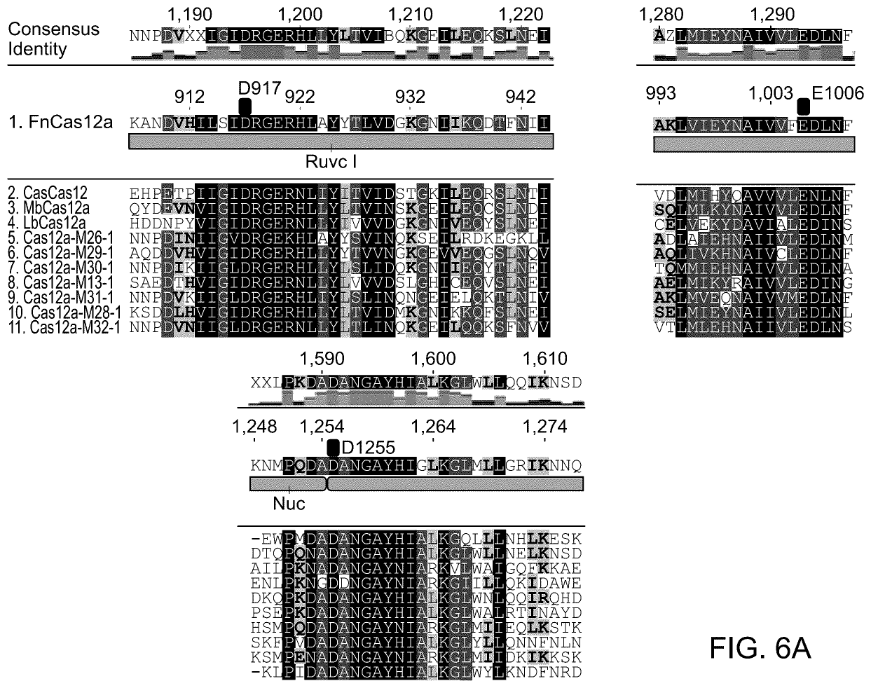

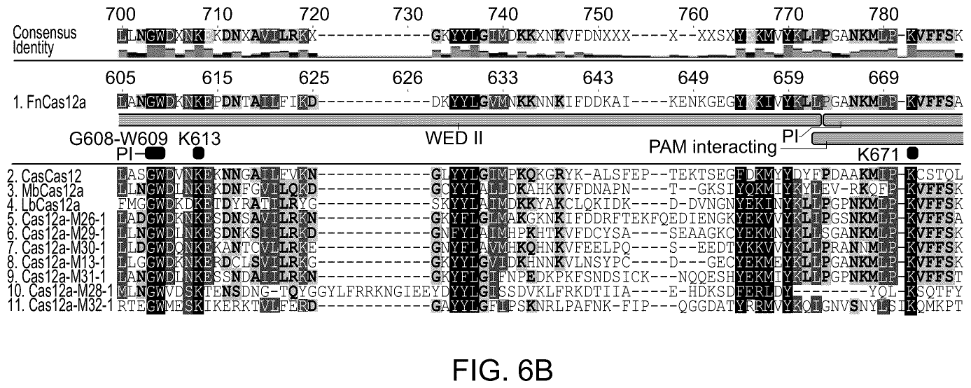

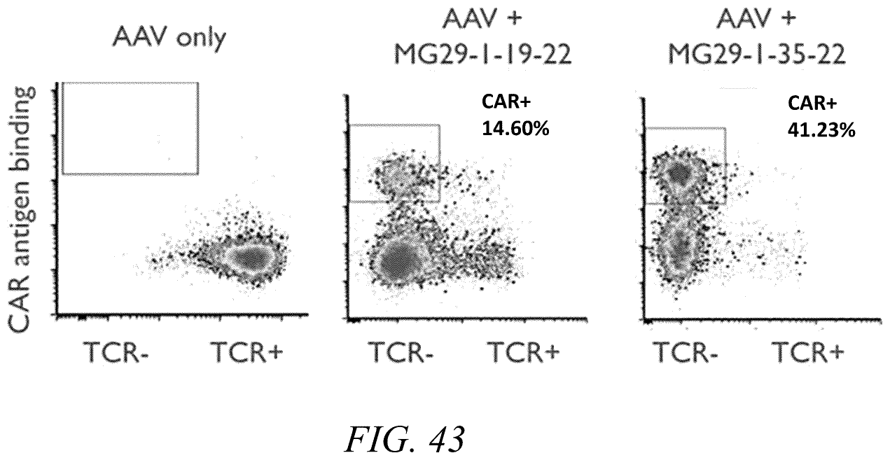

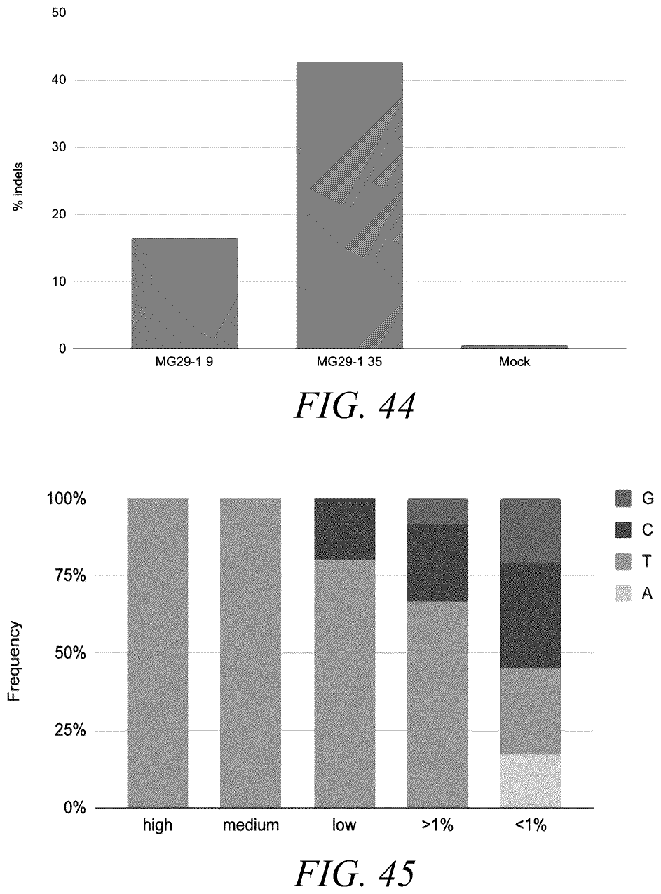

140,867 Mbp of assembled metagenomic sequencing data was mined from diverse environments (soil, thermophilic, sediments, human and non-human microbiomes). In total, 119 genomic fragments encoded CRISPR effectors distantly related to Type V-A nucleases next to a CRISPR array (see FIG. 43). Type V-A effectors were classified into 14 novel families sharing less than 30% average pairwise amino acid identity between each other, and with reference sequences (e.g., LbCas12a, AsCas12a, FnCas12a). Some effectors contained RuvC and alpha-helical recognition domains, as well as conserved DED nuclease catalytic residues from the RuvCI/CII/CIII domains (identified in multiple sequence alignments, see e.g. Table 1A below), suggesting that these effectors were active nucleases (FIG. 5-FIG. 7). The novel Type V-A nucleases range in size from <800 to 1,400 amino acids in length (see FIG. 5A) and their taxonomic classification spanned a diverse array of phyla (see FIG. 4A) suggesting possible horizontal transfer.

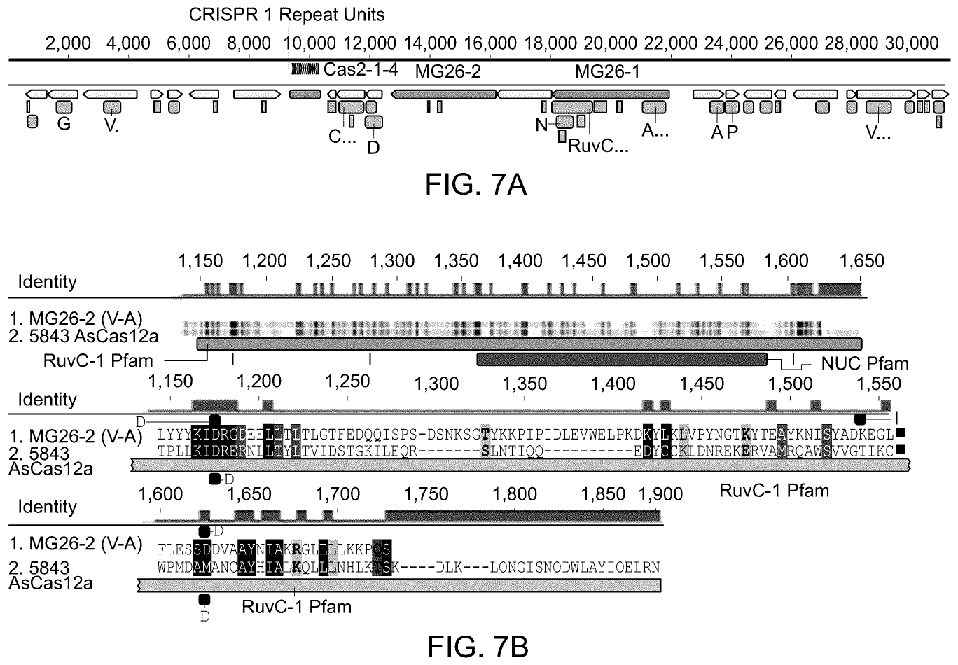

Some genomic fragments carrying a Type V-A CRISPR system also encoded a second effector, referred to here as Type V-A prime (V-A′, FIG. 7A). For example, Type V-A′ MG26-2, which shared 16.6% amino acid identity with the Type V-A MG26-1, was encoded in the same CRISPR Cas operon, and may share the same crRNA with MG26-1 (FIG. 7B). Although no nuclease domains were predicted, MG26-2 contained three RuvC catalytic residues identified from multiple sequence alignments (FIG. 7B).

TABLE 1A
Catalytic residues of Enzymes Described Herein
Identified by Alignment

| MGID | RuvC-I (D) | RuvC-II (E) | RuvC-III (D) |
|---|---|---|---|
| MG84-16 | 238 | 337 | 413 |
| MG84-15 | 238 | 337 | 413 |
| MG84-3 | 230 | 329 | 405 |
| MG84-2 | 230 | 329 | 405 |
| MG84-1 | 230 | 329 | 405 |
| MG84-13 | 233 | 332 | 408 |
| MG84-14 | 233 | 332 | 408 |
| MG84-12 | 233 | 332 | 408 |
| MG84-11 | 233 | 332 | 408 |
| MG84-10 | 233 | 332 | 408 |
| MG84-9 | 233 | 332 | 408 |
| MG84-8 | 233 | 332 | 408 |
| MG84-7 | 233 | 332 | 408 |
| MG84-4 | 233 | 332 | 408 |
| MG84-5 | 233 | 332 | 408 |
| MG84-6 | 233 | 332 | 408 |
| MG81-18 | 296 | 399 | 497 |
| MG81-17 | 296 | 399 | 497 |
| MG81-9 | 297 | 400 | 498 |
| MG81-6 | 297 | 400 | 498 |
| MG81-11 | 297 | 400 | 498 |
| MG81-7 | 297 | 400 | 498 |
| MG81-8 | 297 | 400 | 498 |
| MG81-13 | 297 | 400 | 498 |
| MG81-5 | 300 | 403 | 501 |
| MG81-12 | 300 | 403 | 501 |
| MG81-1 | 300 | 403 | 502 |
| MG81-4 | 310 | 413 | 501 |
| MG81-3 | 310 | 413 | 511 |
| MG81-15 | 388 | 491 | 589 |
| MG81-10 | 310 | 413 | 511 |
| MG81-2 | 306 | 409 | 507 |
| MG90-2 | 388 | 548 | 661 |
| MG91-1 | 444 | 560 | 653 |
| MG91-2 | 245 | 358 | 453 |
| MG91-3 | 297 | 404 | 499 |
| MG37-1 | 763 | 1167 | 1335 |
| MG37-2 | 169 | 538 | 689 |
| MG37-3 | 745 | 1202 | 1350 |
| MG37-4 | 811 | 1230 | 1377 |
| MG37-5 | 775 | 1173 | 1319 |
| MG37-6 | 698 | 1058 | 1229 |
| MG37-7 | 752 | 1135 | 1273 |
| MG53-1 | — | 775 | 920 |
| MG54-1 | — | 612 | 722 |

### Example 3—(General Protocol) PAM Sequence Identification/Confirmation

PAM sequences that can be cleaved in vitro by a CRISPR effector were identified by incubating an effector with a crRNA and a plasmid library having 8 randomized nucleotides located adjacent to the 5′ end of a sequence complementary to the spacer of the crRNA. The plasmid is configured such that if the 8 randomized nucleotides formed a functional PAM sequence, the plasmid was cleaved. Functional PAM sequences were then identified by ligating adapters to the ends of cleaved plasmids and then sequencing DNA fragments comprising the adapters. Putative endonucleases were expressed in an E. coli lysate-based expression system (myTXTL, Arbor Biosciences). An E. coli codon optimized nucleotide sequence encoding the putative nuclease was transcribed and translated in vitro from a PCR fragment under control of a T7 promoter. A second PCR fragment with a minimal CRISPR array composed of a T7 promoter followed by a repeat-spacer-repeat sequence was transcribed in the same reaction. Successful expression of the endonuclease and repeat-spacer-repeat sequence followed by CRISPR array processing provided active in vitro CRISPR nuclease complexes.

A library of target plasmids containing a spacer sequence matching that in the minimal array preceded by 8N (degenerate) bases (potential PAM sequences) was incubated with the output of the TXTL reaction. After 1-3 hours, the reaction was stopped and the DNA was recovered via a DNA clean-up kit, e.g., Zymo DCC, AMPure XP beads, QiaQuick etc. Adapter sequences were blunt-end ligated to DNA fragments with active PAM sequences that had been cleaved by the endonuclease, whereas DNA that had not been cleaved was inaccessible for ligation. DNA segments comprising active PAM sequences were then amplified by PCR with primers specific to the library and the adapter sequence. The PCR amplification products were resolved on a gel to identify amplicons that corresponded to cleavage events. The amplified segments of the cleavage reaction were also used as templates for preparation of an NGS library or as a substrate for Sanger sequencing. Sequencing this resulting library, which was a subset of the starting 8N library, revealed sequences with PAM activity compatible with the CRISPR complex. For PAM testing with a processed RNA construct, the same procedure was repeated except that an in vitro transcribed RNA was added along with the plasmid library and the minimal CRISPR array template was omitted. The following sequences were used as targets in these assays:

(SEQ ID NO: 3860)
CGTGAGCCACCACGTCGCAAGCCT;
(SEQ ID NO: 3861)
GTCGAGGCTTGCGACGTGGTGGCT;
(SEQ ID NO: 3858)
GTCGAGGCTTGCGACGTGGTGGCT;
and
(SEQ ID NO: 3859)
TGGAGATATCTTGAACCTTGCATC.

### Example 4—PAM Sequence Identification/Confirmation for Endonucleases Described Herein

PAM requirements were determined via an E. coli lysate-based expression system (myTXTL, Arbor Biosciences), with modifications. Briefly, the E. coli codon optimized effector protein sequences were expressed under control of a T7 promoter at 29° C. for 16 hours. This crude protein stock was then used in an in vitro digest reaction at a concentration of 20% of the total reaction volume. The reaction was incubated for 3 hours at 37° C. with 5 nM of a plasmid library comprising a constant target sequence preceded by 8N mixed bases, and 50 nM of in vitro transcribed crRNA derived from the same CRISPR locus as the effector linked to a sequence complementary to the target sequence in NEB buffer 2.1 (New England Biolabs; NEB buffer 2.1 was selected in order to compare candidates with commercially available proteins). Protein concentration was not normalized in PAM discovery assays (PCR amplification signal provides high sensitivity for low expression or activity). The cleavage products from the TXTL reactions were recovered via clean up with AMPure SPRI beads (Beckman Coulter). The DNA was blunted via addition of Klenow fragments and dNTPs (New England Biolabs). Blunt-end products were ligated with a 100-fold excess of double stranded adapter sequences and used as template for the preparation of an NGS library, from which PAM requirements were determined from sequence analysis.

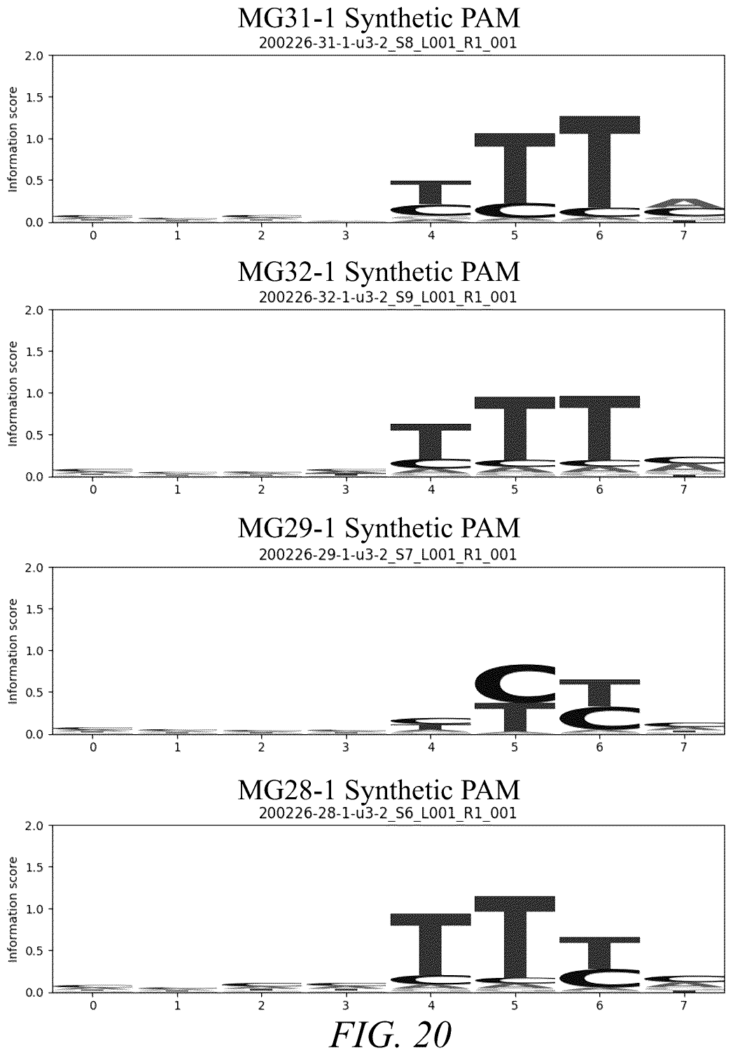

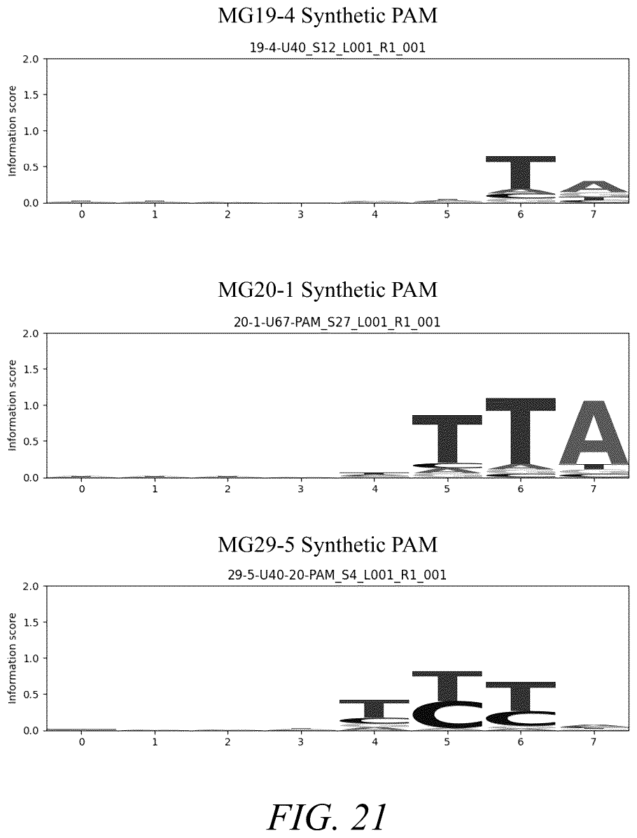

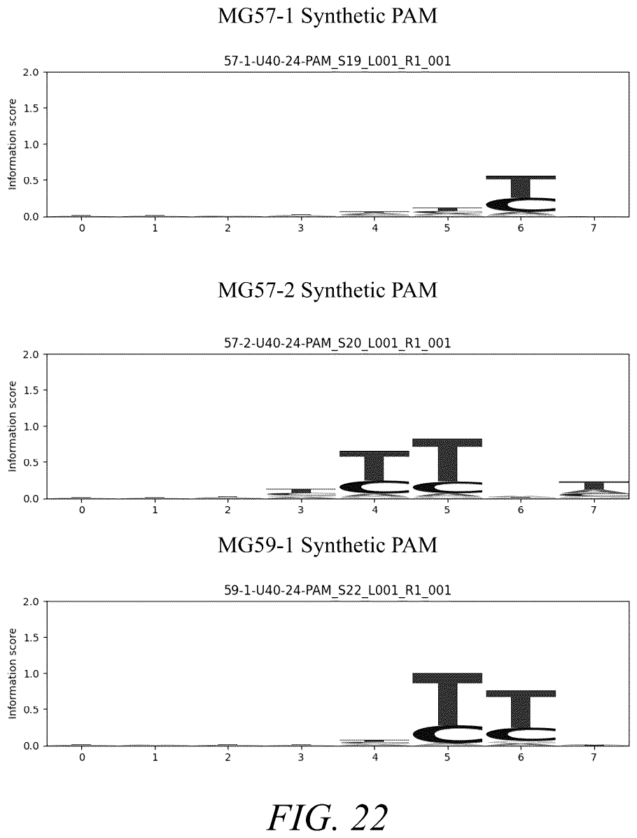

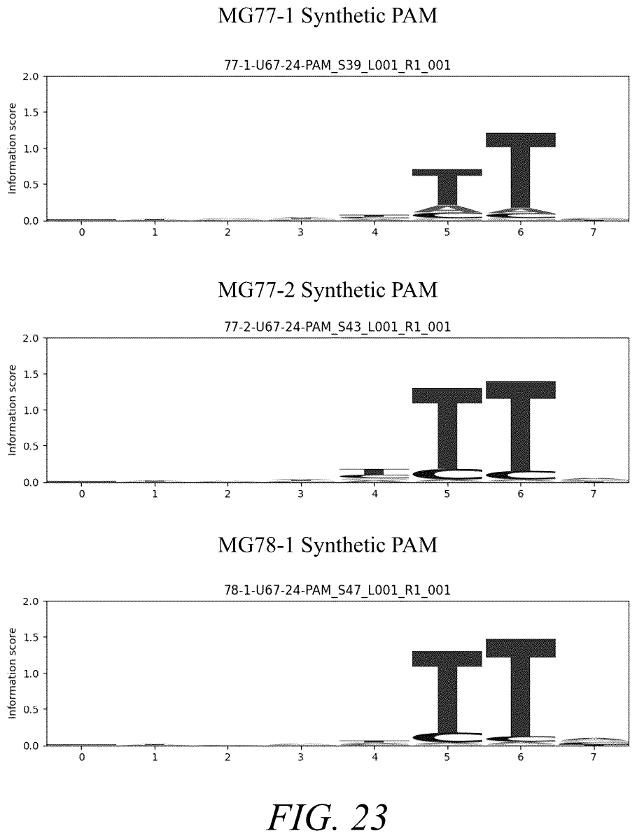

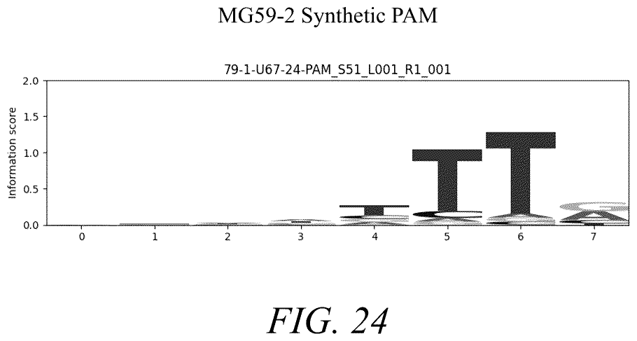

Raw NGS reads were filtered by Phred quality score >20. The 28 bp representing the identified DNA sequence from the backbone adjacent to the PAM was used as a reference to find the PAM-proximal region and the 8 bp adjacent were identified as the putative PAM. The distance between the PAM and the ligated adapter was also measured for each read. Reads that did not have an exact match to the reference sequence or adapter sequence were excluded. PAM sequences were filtered by cut site frequency such that PAMs with the most frequent cut site f2 bp were included in the analysis. This correction removed low levels of background cleavage that may occur at random positions due to the use of crude E. coli lysate. This filtering stage can remove between 2% and 40% of the reads depending on the signal to noise ratio of the candidate protein, where less active proteins have more background signal. For reference MG29-1, 2% of reads were filtered out at this stage. The filtered list of PAMs was used to generate a sequence logo using Logomaker. These sequence logo depictions of PAMs are presented in FIGS. 20-24.

### Example 5—tracrRNA Prediction and Guide Design

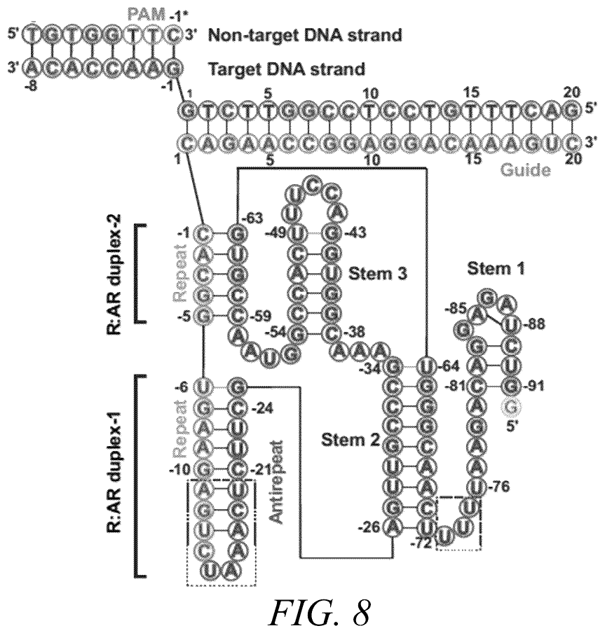

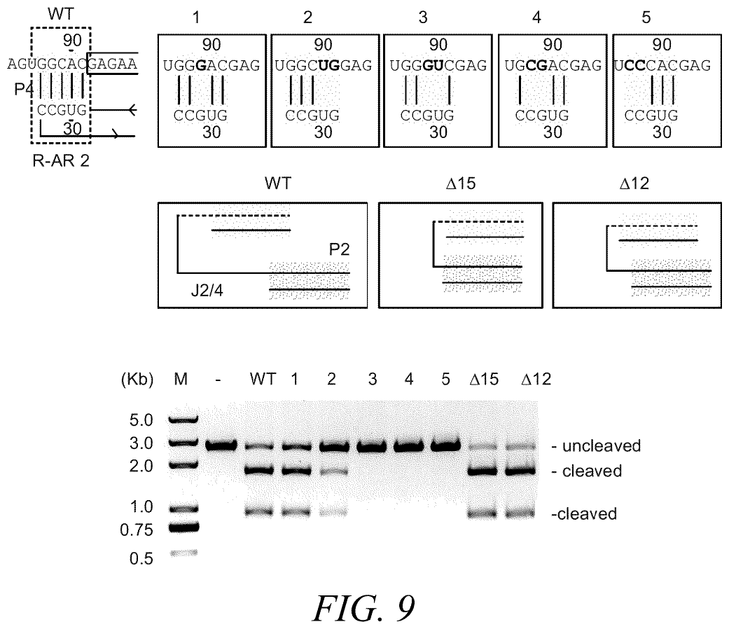

The crystal structure of a ternary complex of AacC2c1 (Cas12b) bound to a sgRNA and a target DNA reveals two separate repeat-anti-repeat (R-AR) motifs in the bound sgRNA, denoted R-AR duplex 1 and R-AR duplex 2 (see FIG. 8 and FIG. 9 herein and Yang, Hui, Pu Gao, Kanagalaghatta R. Rajashankar, and Dinshaw J. Patel. 2016. “PAM-Dependent Target DNA Recognition and Cleavage by C2c1 CRISPR-Cas Endonuclease.” Cell 167 (7): 1814-28.e12 and Liu, Liang, Peng Chen, Min Wang, Xueyan Li, Jiuyu Wang, Maolu Yin, and Yanli Wang. 2017. “C2c1-sgRNA Complex Structure Reveals RNA-Guided DNA Cleavage Mechanism.” Molecular Cell 65 (2): 310-22, each of which is incorporated by reference herein in its entirety). Putative tracrRNA sequences for the CRISPR effectors disclosed herein were identified by searching for anti-repeat sequences in the surrounding genomic context of native CRISPR arrays, where the R-AR duplex 2 anti-repeat sequence occurs ˜20-90 nucleotides upstream of (closer to the 5′ end of the tracrRNA than) the R-AR duplex 1 anti-repeat sequence. Following tracrRNA sequence identification, two guide sequences were designed for each enzyme. The first included both R-AR duplexes 1 & 2 (see for example SEQ ID NOs: 3636, 3640, 3644, 3648, 3652, 3656, 3660, 3671, and 3672), and the second was a shorter guide sequence with the R-AR duplex 1 region deleted (see e.g., SEQ ID NOs: 3637, 3641, 3645, 3649, 3653, 3657, and 3661), as this region may not be essential for cleavage.

### Example 6—Protocol for Predicted RNA Folding

Predicted RNA folding of RNA sequences at 37° C. was computed using the method of Andronescu 2007 (which is entirely incorporated by reference herein).

### Example 7—RNA Guide Identification

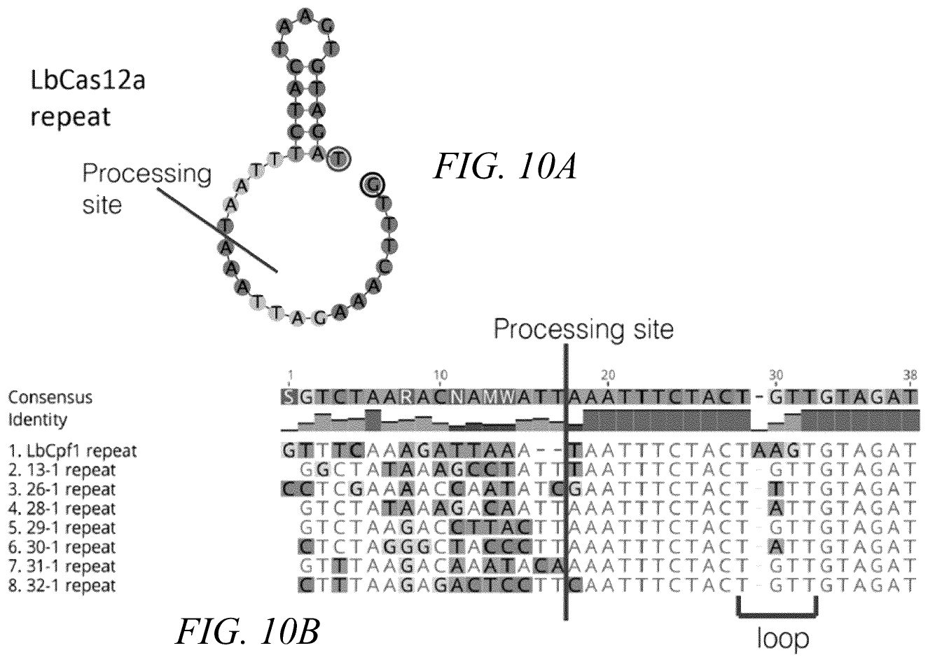

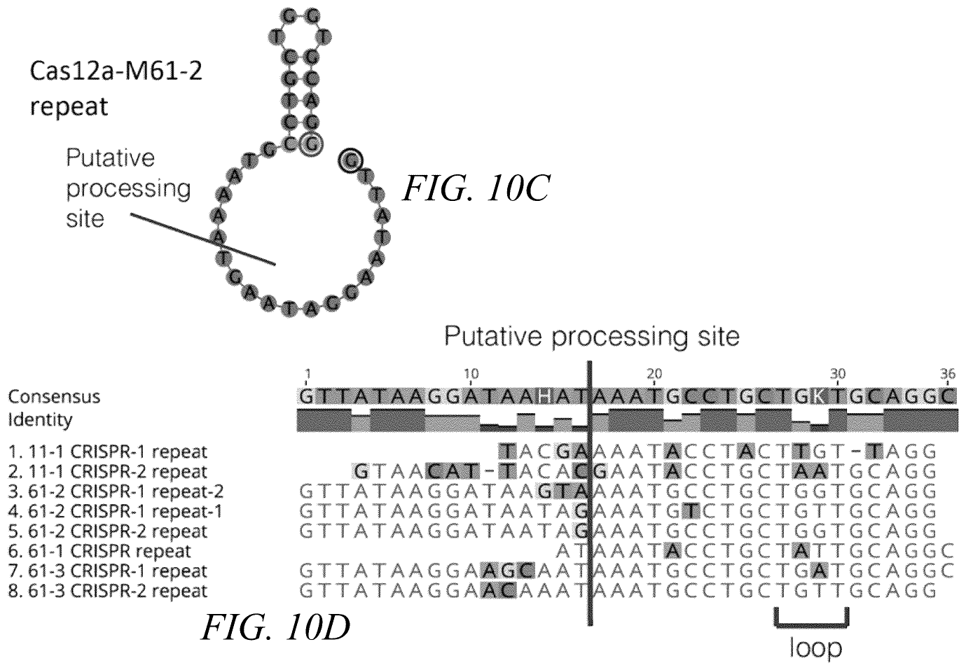

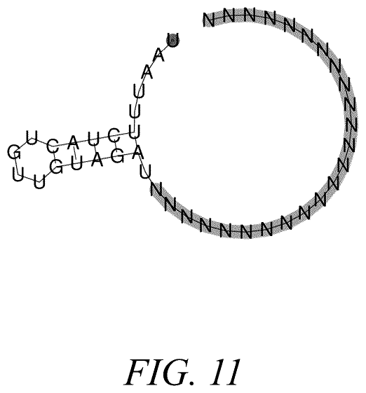

For contigs that encoded a Type V-A effector and a CRISPR array, secondary structure folding of repeats indicated that the novel Type V-A systems require a single guide crRNA (sgRNA, FIGS. 10A-D). No tracrRNA sequences were identified. The sgRNA contained ˜19-22 nt from the 3′ end of the CRISPR repeat. A multiple sequence alignment of CRISPR repeats from six of the Type V-A candidates that were tested for in-vitro activity shows a highly conserved motif at the 3′ end of the repeat, which formed the stem-loop structure of the sgRNA (FIG. 10C). The motif, UCUAC[N3-5]GUAGAU, comprised short palindromic repeats (the stem) separated by between three and five nucleotides (the loop).

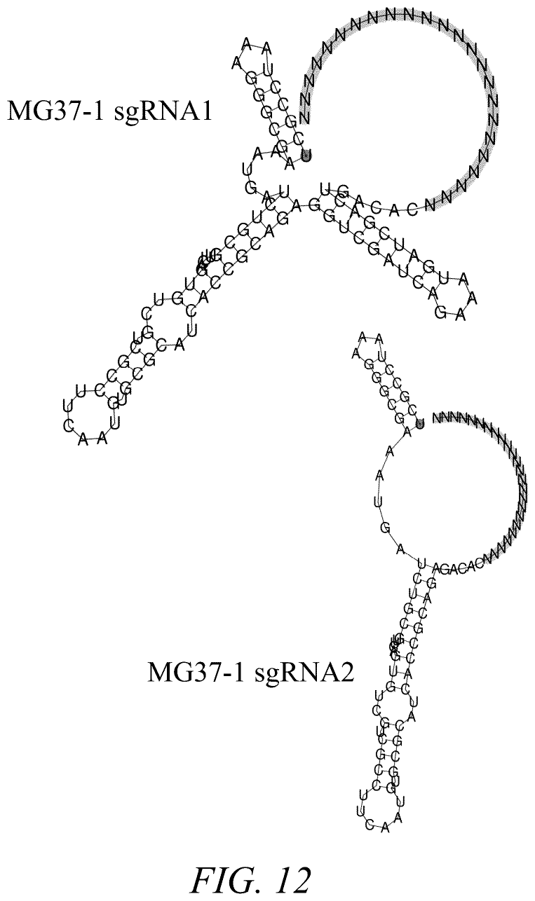

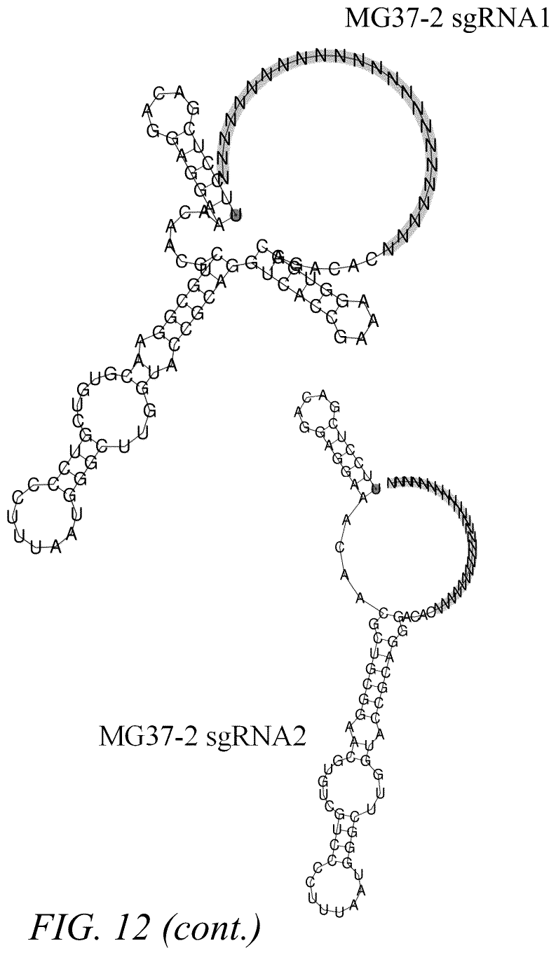

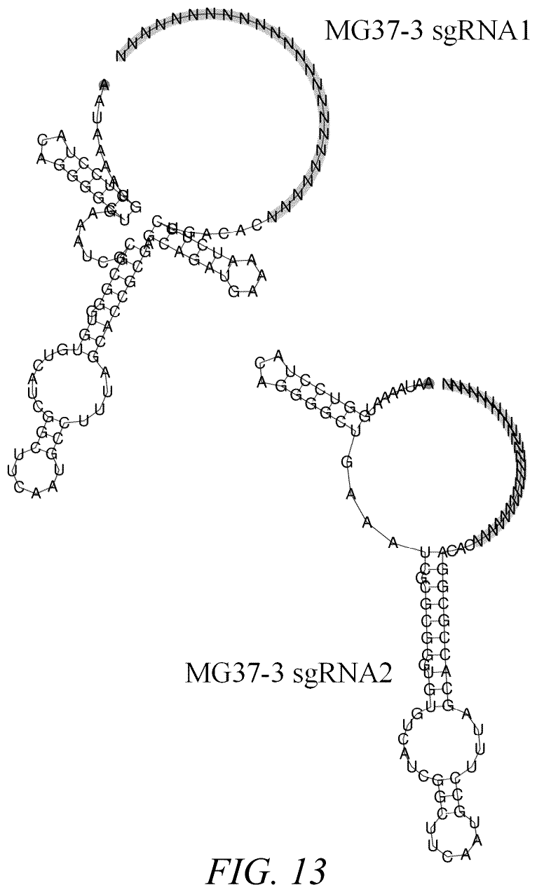

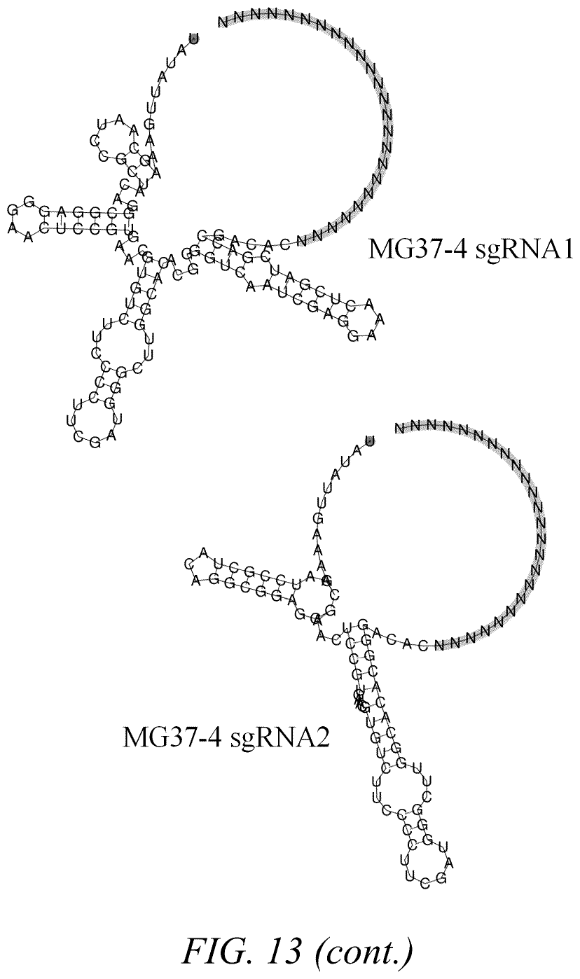

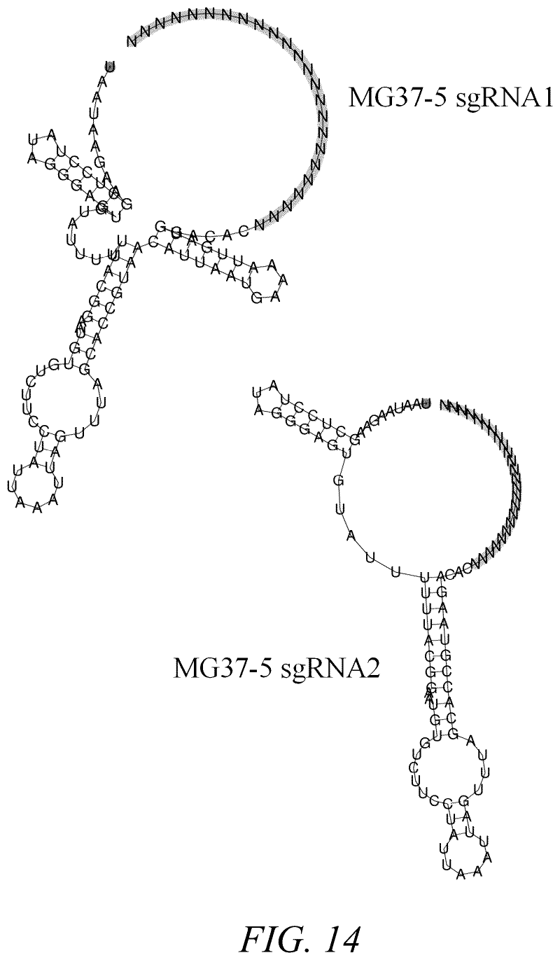

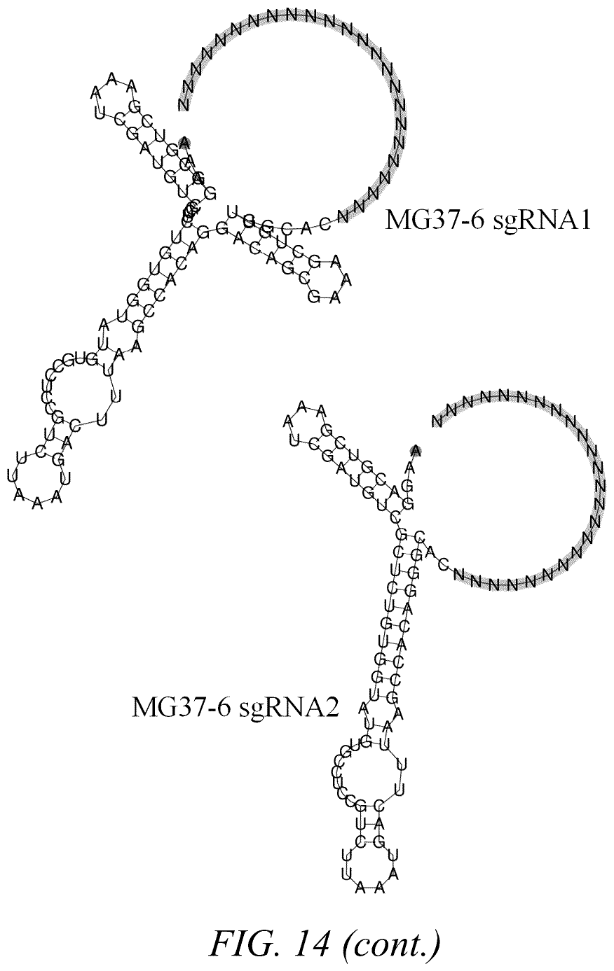

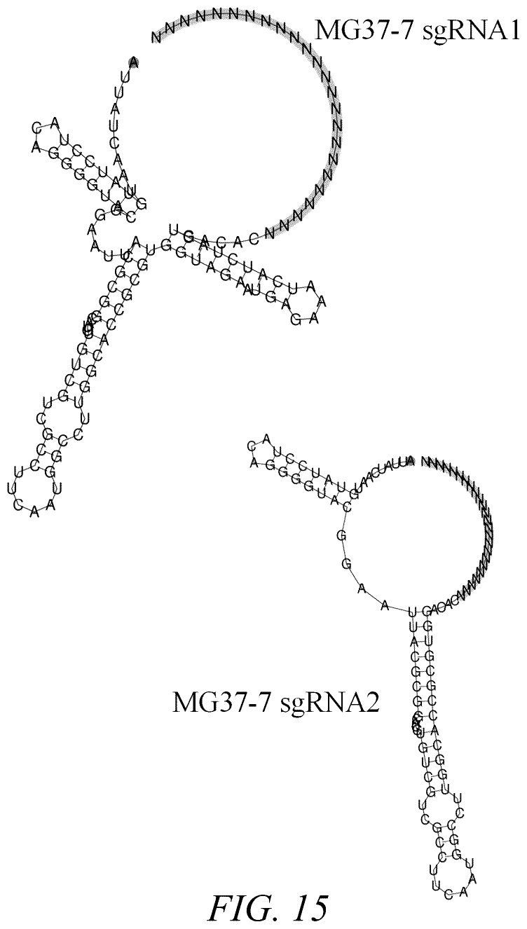

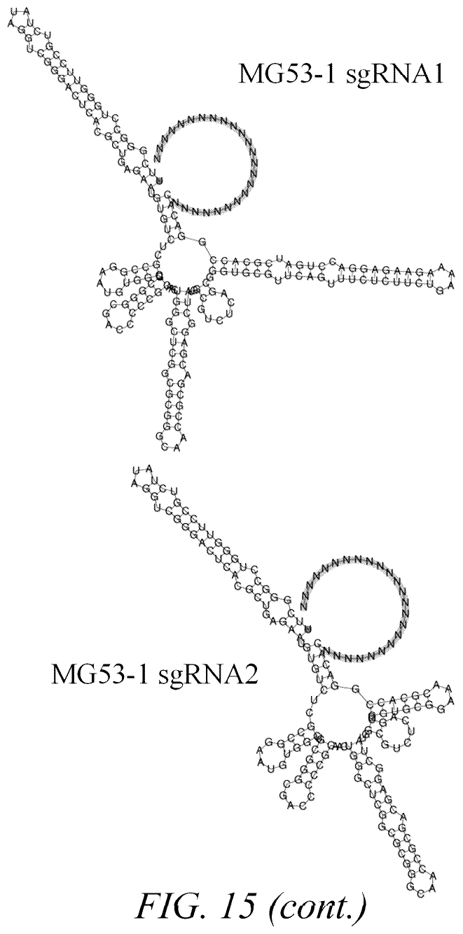

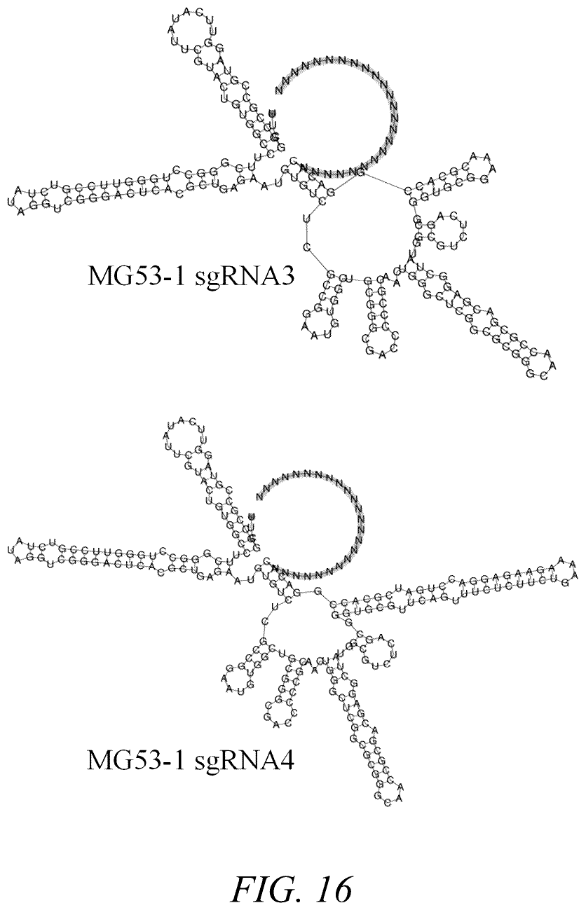

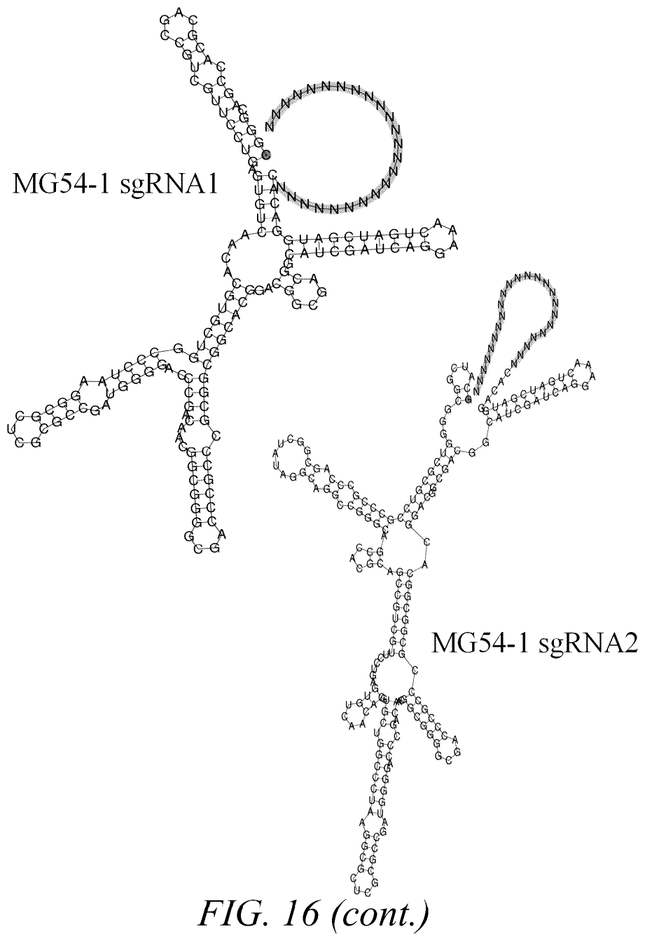

The conservation of the sgRNA motif was used to uncover novel effectors that may not show similarity to classified Type V-A nucleases. Motifs were searched in repeats from 69,117 CRISPR arrays. The most common motif contained a 4-nucleotide loop, while 3- and 5-nucleotide loops were less common (see FIG. 12, FIG. 13, FIG. 14, FIG. 15, and FIG. 16). Inspection of the genomic context surrounding the CRISPR arrays containing the repeat motif revealed numerous effectors of varying lengths. For example, effectors of the family MG57 were the largest of the Type V-A nucleases identified (average ˜1400 aa), and encoded a repeat with a 4-bp loop. Another family identified from HMM analysis contained a different repeat motif, CCUGC[N₃₋₄]GCAGG (see FIGS. 5C,5D). Although differing in sequence, the structure was predicted to fold into a highly similar stem-loop structure.

### Example 8—In Vitro Cleavage Efficiency of MG CRISPR Complexes

Endonucleases are expressed as His-tagged fusion proteins from an inducible T7 promoter in a protease deficient E. coli B strain. Cells expressing the His-tagged proteins are lysed by sonication and the His-tagged proteins purified by Ni-NTA affinity chromatography on a HisTrap FF column (GE Lifescience) on an AKTA Avant FPLC (GE Lifescience). The eluate is resolved by SDS-PAGE on acrylamide gels (Bio-Rad) and stained with InstantBlue Ultrafast coomassie (Sigma-Aldrich). Purity is determined using densitometry of the protein band with ImageLab software (Bio-Rad). Purified endonucleases are dialyzed into a storage buffer composed of 50 mM Tris-HCl, 300 mM NaCl, 1 mM TCEP, 5% glycerol; pH 7.5 and stored at −80° C. Target DNAs containing spacer sequences and PAM sequences (determined for example as in either Example 3 or Example 4) are constructed by DNA synthesis. A single representative PAM is chosen for testing when the PAM has degenerate bases. The target DNAs are comprised of 2200 bp of linear DNA derived from a plasmid via PCR amplification with a PAM and spacer located 700 bp from one end. Successful cleavage results in fragments of 700 and 1500 bp. The target DNA, in vitro transcribed single RNA, and purified recombinant protein are combined in cleavage buffer (10 mM Tris, 100 mM NaCl, 10 mM MgCl₂) with an excess of protein and RNA and are incubated for 5 minutes to 3 hours, usually 1 hr. The reaction is stopped via addition of RNAse A and incubation at 60 minutes. The reaction is then resolved on a 1.2% TAE agarose gel and the fraction of cleaved target DNA is quantified in ImageLab software.

### Example 9—Testing of Genome Cleavage Activity of MG CRISPR Complexes in E. coli

E. coli lacks the capacity to efficiently repair double-stranded DNA breaks. Thus, cleavage of genomic DNA can be a lethal event. Exploiting this phenomenon, endonuclease activity is tested in E. coli by recombinantly expressing an endonuclease and a guide RNA (determined for example as in Example 6) in a target strain with spacer/target and PAM sequences integrated into its genomic DNA (determined for example as in Example 4) integrated into their genomic DNA are transformed with DNA encoding the endonuclease. Transformants are then made chemocompetent and are transformed with 50 ng of guide RNAs (e.g., crRNAs) either specific to the target sequence (“on target”), or non-specific to the target (“non target”). After heat shock, transformations were recovered in SOC for 2 hours at 37° C. Nuclease efficiency is then determined by a 5-fold dilution series grown on induction media. Colonies are quantified from the dilution series in triplicate. A reduction in the number of colonies transformed with an on-target guide RNA compared to the number of colonies transformed with an off-target guide RNA indicates specific genome cleavage by the endonuclease.

### Example 10—Generic Procedure: Testing of Genome Cleavage Activity of MG CRISPR Complexes in Mammalian Cells

Two types of mammalian expression vectors are used to detected targeting and cleavage activity in mammalian cells. In the first, the MG Cas effector is fused to a C-terminal SV40 NLS and a viral 2A consensus cleavable peptide sequence linked to a GFP tag (the 2A-GFP tag to monitor expression of the protein). In the second, the MG Cas effector is fused to two SV40 NLS sequences, one on the N-terminus and the other on the C-terminus. The NLS sequences comprise any of the NLS sequences described herein (for example SEQ ID NOs: 3938-3953). In some instances, nucleotide sequences encoding the endonucleases are codon-optimized for expression in mammalian cells.

A single guide RNA with a crRNA sequence fused to a sequence complementary to a mammalian target DNA is cloned into a second mammalian expression vector. The two plasmids are co-transfected into HEK293T cells. 72 hours after co-transfection, DNA is extracted from the transformed HEK293T cells and used for the preparation of an NGS-library. Percent NHEJ is measured by quantifying indels at the target site to demonstrate the targeting efficiency of the enzyme in mammalian cells. At least 10 different target sites are chosen to test each protein's activity.

### Example 11—Testing of Genome Cleavage Activity of MG CRISPR Complexes in Mammalian Cells

To show targeting and cleavage activity in mammalian cells, the MG Cas effector protein sequences were cloned into a mammalian expression vector with flanking N and C-terminal SV40 NLS sequences, a C-terminal His tag, and a 2A-GFP (e.g. a viral 2A consensus cleavable peptide sequence linked to a GFP) tag at the C terminus after the His tag (Backbone 1). In some instances, nucleotide sequences encoding the endonucleases were the native sequence, codon-optimized for expression in E. coli cells or codon-optimized for expression in mammalian cells.

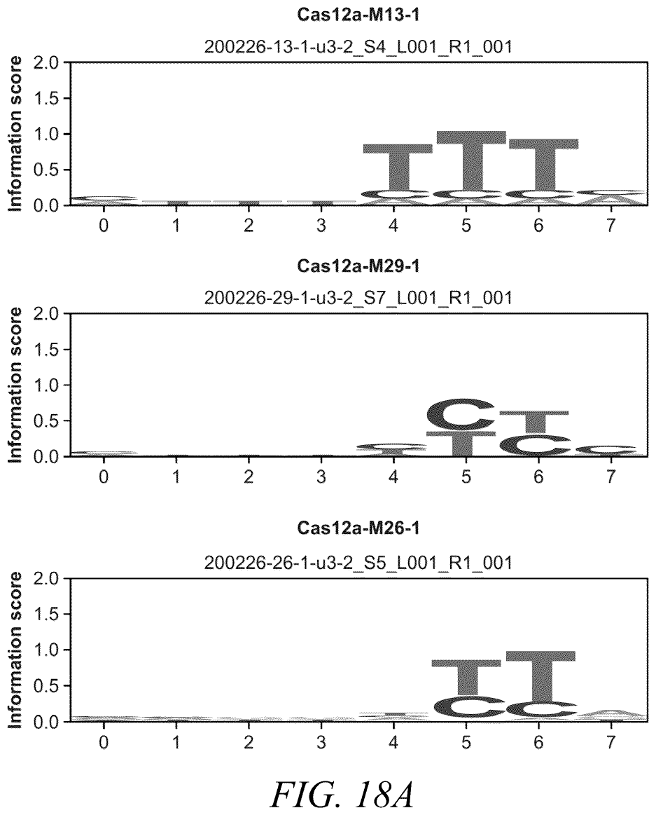

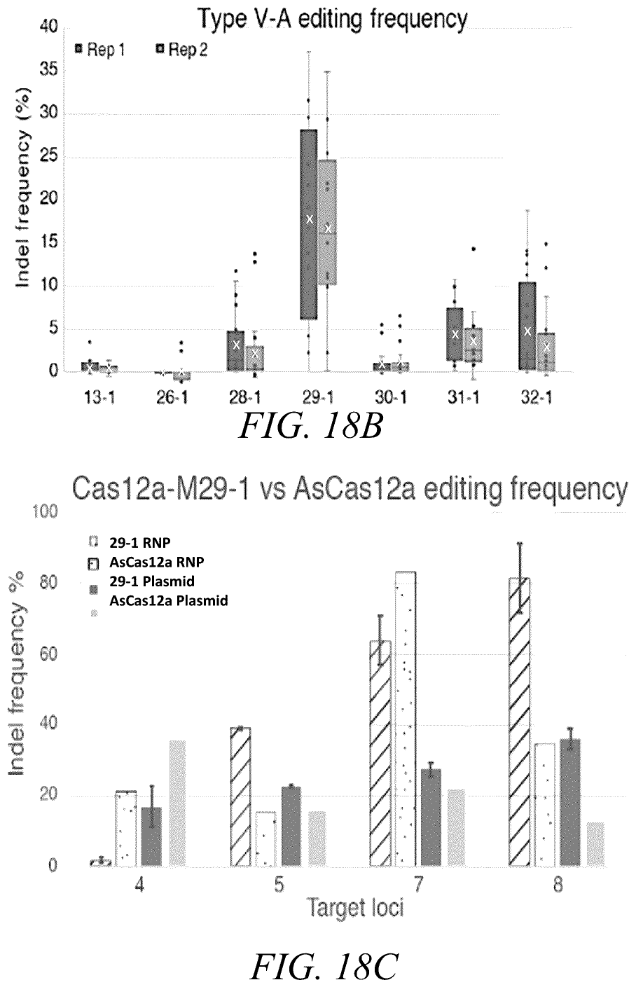

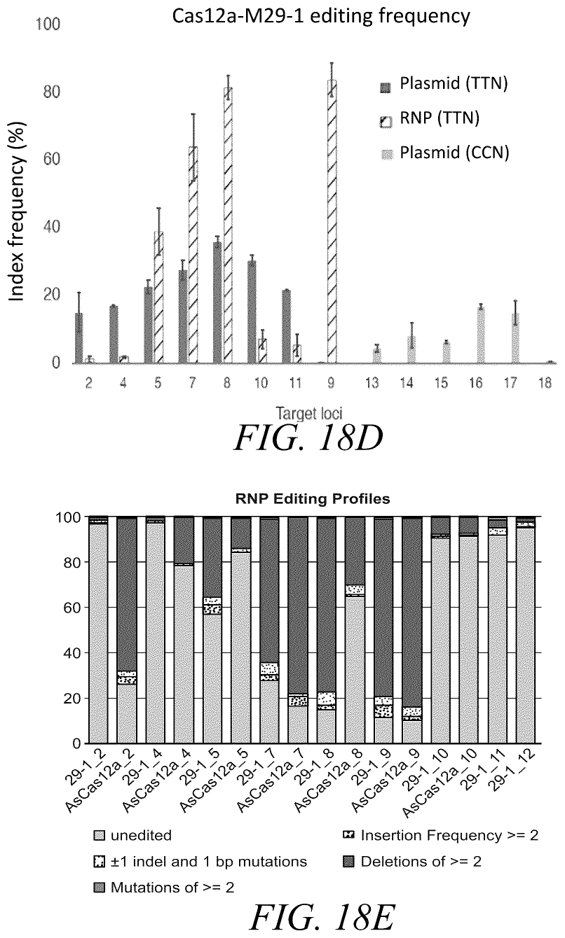

The single guide RNA sequence (sgRNA) with a gene target of interest was also cloned into a mammalian expression vector. The two plasmids are co-transfected into HEK293T cells. 72 hours after co-transfection of the expression plasmid and a sgRNA targeting plasmid into HEK293T cells, the DNA was extracted and used for the preparation of an NGS-library. Percent NHEJ was measured via indels in the sequencing of the target site to demonstrate the targeting efficiency of the enzyme in mammalian cells. 7-12 different target sites were chosen for testing each protein's activity. An arbitrary threshold of 5% indels was used to identify active candidates. Genome editing efficiency in human cells was assessed from the NGS reads with CRISPResso using parameters: cleavage offset=−4 and window=10. All post cleavage events from the CRISPResso output were summed for ±1 bp indels/mutations, and ≥2 bp deletions, insertions, and mutations. All outcomes were normalized to total sequences aligned to the expected amplicon (see FIGS. 18A-E)

### Example 12—Characterization of Mg29 Family

PAM Specificity, tracrRNA/sgRNA Validation

The targeted endonuclease activity of MG29 family endonuclease systems was confirmed using the myTXTL system described in Example 3 and Example 8. In this assay, PCR amplification of cleaved target plasmids yields a product that migrates at approximately 170 bp in the gel, as shown in FIGS. 17A-B. Amplification products were observed for MG29-1 with crRNA corresponding to SEQ ID NO: 3609 (see FIG. 17A, lane 7). Sequencing the PCR products revealed active PAM sequences for these enzymes as shown in Table 2 below.

TABLE 2
Activity of MG29-1 at various target sites

| target | | 5′ sequence | | %NHEJ |
|---|---|---|---|---|
| ID | target sequence | including PAM | locus | (mean ± std) |
| target1 | TGTCAGAAGCAAATGTAAGCAATA | AACACAGTTG | HBB | 2.185 ± 0.007 |
| | (SEQ ID NO: 3914) | (SEQ ID NO: 3890) | | |
| target2 | CTGAAAGGTTATTGTTGTGTTTGT | TACAGTTTTG | Fibrinogen | 10.5 ± 8.74 |
| | (SEQ ID NO: 3915) | (SEQ ID NO: 3891) | | |
| target3 | CTAGTGAACACAGTTGTGTCAGAA | TTTGAGGTTG | HBB | 2.14 ± 2.83 |
| | (SEQ ID NO: 3916) | (SEQ ID NO: 3892) | | |
| target4 | TGAAGTCTTACAAGGTTATCTTAT | TTTGTATTTG | Albumin | 13.757 ± 5.46 |
| | (SEQ ID NO: 3917) | (SEQ ID NO: 3893) | | |
| target5 | CACTTTCCTTAGTGCGCAAAAGAA | AGTTACTTTG | Albumin | 17.937 ± 8.27 |
| | (SEQ ID NO: 3918) | (SEQ ID NO: 3894) | | |
| target6 | GTGGTGAGGCCCTGGGCAGGTTGG | GATGAAGTTG | HBB | 12.545 ± 1.73 |
| | (SEQ ID NO: 3919) | (SEQ ID NO: 3895) | | |
| target7 | GGAGGTCAGAAATAGGGGGTCCAG | TAGCTGTTTG | VEGFA | 23.56 ± 7.04 |
| | (SEQ ID NO: 3920) | (SEQ ID NO: 3896) | | |
| target8 | GAAAGGGGGTGGGGGGAGTTTGCT | ATGGGCTTTG | VEGFA | 30.147 ± 10.17 |
| | (SEQ ID NO: 3921) | (SEQ ID NO: 3897) | | |
| target9 | GTATCAAGGTTACAAGACAGGTTT | GGGCAGGTTG | HBB | 10.935 ± 1.56 |
| | (SEQ ID NO: 3922) | (SEQ ID NO: 3898) | | |
| target10 | TGTGAGGGAGCACCGTTCTCTAGA | TACATAGTTG | Apolipoprotein | 30.43 ± 1.57 |
| | (SEQ ID NO: 3923) | (SEQ ID NO: 3899) | | |
| target11 | GGTAGTTTTCTGTGGTCCTATTAT | TACGCATTTG | Apolipoprotein | 18.173 ± 6.28 |
| | (SEQ ID NO: 3924) | (SEQ ID NO: 3900) | | |
| target12 | CCAGGAAAGTTGATGTGGTCTGCG | CCGCAAGTTG | Apolipoprotein | 7.47 ± 10.52 |
| | (SEQ ID NO: 3925) | (SEQ ID NO: 3901) | | |

### Targeted Endonuclease Activity in Mammalian Cells

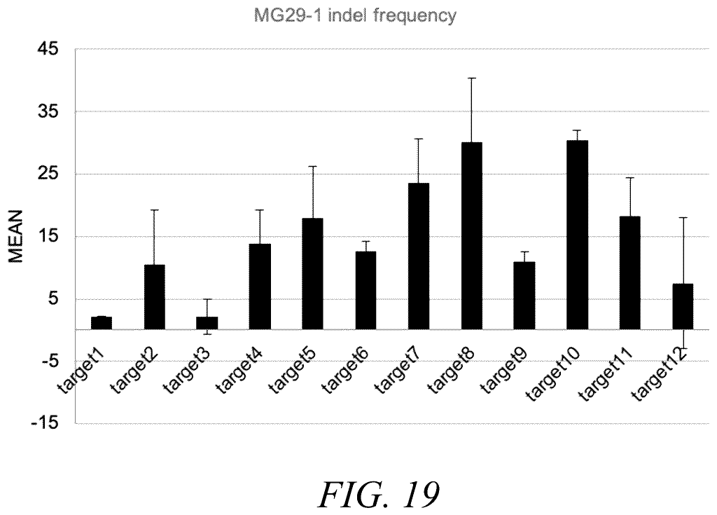

MG29-1 target loci were chosen to test locations in the genome with the PAM YYn (Sequence Number: A3871). The spacers corresponding to the chosen target sites were cloned into the sgRNA scaffold in the mammalian vector system backbone 1 described in Example 9. The sites are listed in Table 3 below. The activity of MG29-1 at various target sites is shown in Table 2 and FIG. 19.

TABLE 3
5′ PAM Sequences and crRNAs for Enzymes Described Herein

| | Enzyme | | PAM | crRNA |
|---|---|---|---|---|
| | SEQ ID | | Sequence | SEQ ID |
| Enzyme | NO: | 5′ PAM | Number: | NO: |
| MG29-1 | 215 | KTTG | A3870 | 3608 |

### Example 13—High-Replicate PAM Determination Via NGS

Type V endonucleases (e.g. MG28, MG29, MG30, MG31 endonucleases) were tested for cleavage activity using E. coli lysate-based expression in the myTXTL kit as described in Example 3 and Example 8. Upon incubation with a crRNA and a plasmid library containing a spacer sequencing matching the crRNA preceded by 8 degenerated (“N”) bases (a 5′ PAM library), the subset of the plasmid library with a functional PAM was cleaved. Ligation to this cut site and PCR amplification provided evidence of activity, demonstrated by the bands observed in the gel at 170 bp (FIG. 17B). Gel 1 (top panel, A) lanes are as follows: 1 (ladder; darkest band corresponds to 200 bp); 2: positive control (previously verified library); 3 (n/a); 4 (n/a); 5 (MG28-1); 6 (MG29-1); 7 (MG30-1); 8 (MG31-1); 9 (MG32-1); and 10 (Ladder). Gel 2 (bottom panel, B) lanes are as follows: 1 (ladder; darkest band corresponds to 200 bp); 2 (LbCpf1 positive control); 3 (LbCpf1 positive control); 4 (negative control); 5 (n/a); 6 (n/a); 7 (MG28-1); 8 (MG29-1); 9 (MG30-1); 10 (MG31-1); 11 (MG32-1).

The PCR products were further subjected to NGS sequencing and the PAMs were collated into seqLogo (see e.g., Huber et al. Nat Methods. 2015 Feb.; 12(2):115-21, which is incorporated by reference herein) representations (FIG. 20). The seqLogo representation shows the 8 bp which are upstream (5′) of the spacer labeled as positions 0-7. As shown in the FIG. 20, the PAMs are pyrimidine rich (C and T), with most sequence requirements 2-4 bp upstream of the spacer (positions 4-6 in the SeqLogo).

The PAMs for the MG candidates are shown in Table 4 below.

TABLE 4
5′ PAM Sequences and crRNAs for Enzymes Described Herein

| | Enzyme | | PAM | crRNA |
|---|---|---|---|---|
| | SEQ ID | | Sequence | SEQ ID |
| Enzyme | NO: | 5′ PAM | Number: | NO: |
| MG28-1 | 141 | TTTn | A3868 | 3609 |
| MG29-1 | 215 | YYn | A3871 | 3609 |
| MG31-1 | 229 | YTTn | A3875 | 3609 |
| MG32-1 | 261 | TTTn | A3877 | 3609 |

In some cases, the position immediately adjacent to the spacer may have a weaker specificity, e.g. for “m” or “v” instead of “n”.

### Example 14—Targeted Endonuclease Activity in Mammalian Cells with MG31 Nucleases

### Targeted Endonuclease Activity in Mammalian Cells

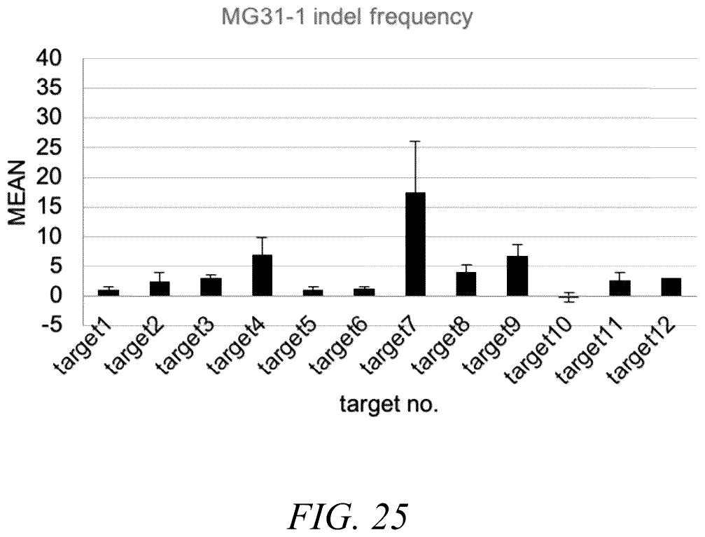

MG31-1 target loci were chosen to test locations in the genome with the PAM TTTR (Sequence Number: A3875). The spacers corresponding to the chosen target sites were cloned into the sgRNA scaffold in the mammalian vector system backbone 1 described in Example 11. The sites are listed in Table 5 below. The activity of MG31-1 at various target sites is shown in Table 5 and FIG. 25.

TABLE 5
Activity of MG31-1 at various target sites

| | | | | %NHEJ |
|---|---|---|---|---|
| target ID | target sequence | PAM | locus | (mean = std) |
| target1 | GTTATTAATTTCTTGCTACTTGTC | GTTTTCTTTA | Fibrinogen | 1.005 ± 0.516 |
| | (SEQ ID NO: 3926) | (SEQ ID NO: | | |
| | | 3902) | | |
| target2 | CTGAAAGGTTATTGTTGTGTTTGT | TACAGTTTTG | Fibrinogen | 2.417 ± 1.47 |
| | (SEQ ID NO: 3927) | (SEQ ID NO: | | |
| | | 3903) | | |
| target3 | GTGTTAGTACAGTTTTGCTGAAAG | AGAACTTTTA | Fibrinogen | 2.925 ± 0.516 |
| | (SEQ ID NO: 3928) | (SEQ ID NO: | | |
| | | 3904) | | |
| target4 | TGAAGTCTTACAAGGTTATCTTAT | TTTGTATTTG | Albumin | 7.053 ± 2.72 |
| | (SEQ ID NO: 3929) | (SEQ ID NO: | | |
| | | 3905) | | |
| target5 | CACTTTCCTTAGTGCGCAAAAGAA | AGTTACTTTG | Albumin | 0.927 ± 0.50 |
| | (SEQ ID NO: 3930) | (SEQ ID NO: | | |
| | | 3906) | | |
| target6 | CCTAGGATGTTTGAATTTTATTAA | TTTTTTTTTA | Albumin | 1.125 ± 0.43 |
| | (SEQ ID NO: 3931) | (SEQ ID NO: | | |
| | | 3907) | | |
| target7 | GGAGGTCAGAAATAGGGGGTCCAG | TAGCTGTTTG | VEGFA | 17.39 ± 8.67 |
| | (SEQ ID NO: 3932) | (SEQ ID NO: | | |
| | | 3908) | | |
| target8 | GAAAGGGGGTGGGGGGAGTTTGCT | ATGGGCTTTG | VEGFA | 4.01 ± 1.29 |
| | (SEQ ID NO: 3933) | (SEQ ID NO: | | |
| | | 3909) | | |
| target9 | GCCAGAGCCGGGGTGTGCAGACGG | TCCCTCTTTA | VEGFA | 6.72 ± 1.92 |
| | (SEQ ID NO: 3934) | (SEQ ID NO: | | |
| | | 3910) | | |
| target10 | CTTGGACCTTGTTTTGCTTACTGT | ACAAATTTTA | Apolipoprotein | −0.32 ± 0.75 |
| | (SEQ ID NO: 3935) | (SEQ ID NO: | | |
| | | 3911) | | |
| target11 | GGTAGTTTTCTGTGGTCCTATTAT | TACGCATTTG | Apolipoprotein | 2.593 ± 1.33 |
| | (SEQ ID NO: 3936) | (SEQ ID NO: | | |
| | | 3912) | | |
| target12 | ATCATAAGAAGTTAGCTTGACGCA | GAAAAATTTA | Apolipoprotein | 3.095 |
| | (SEQ ID NO: 3937) | (SEQ ID NO: | | |
| | | 3913) | | |

### Example 3—In Vitro Activity

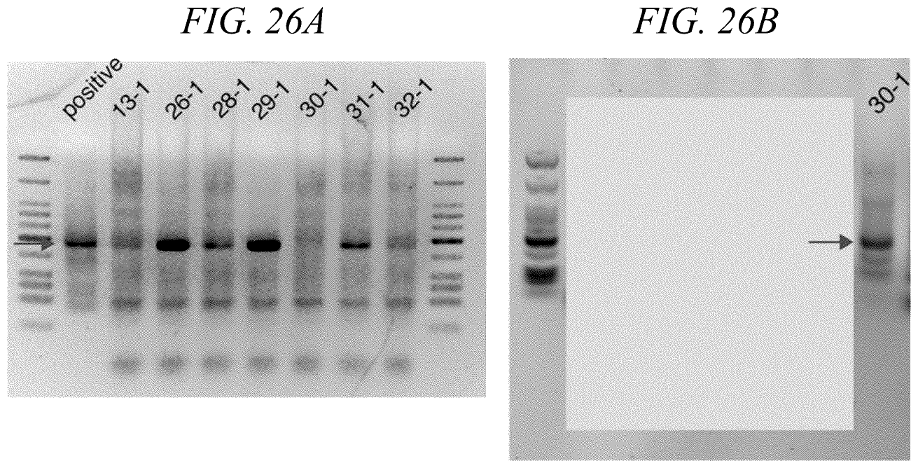

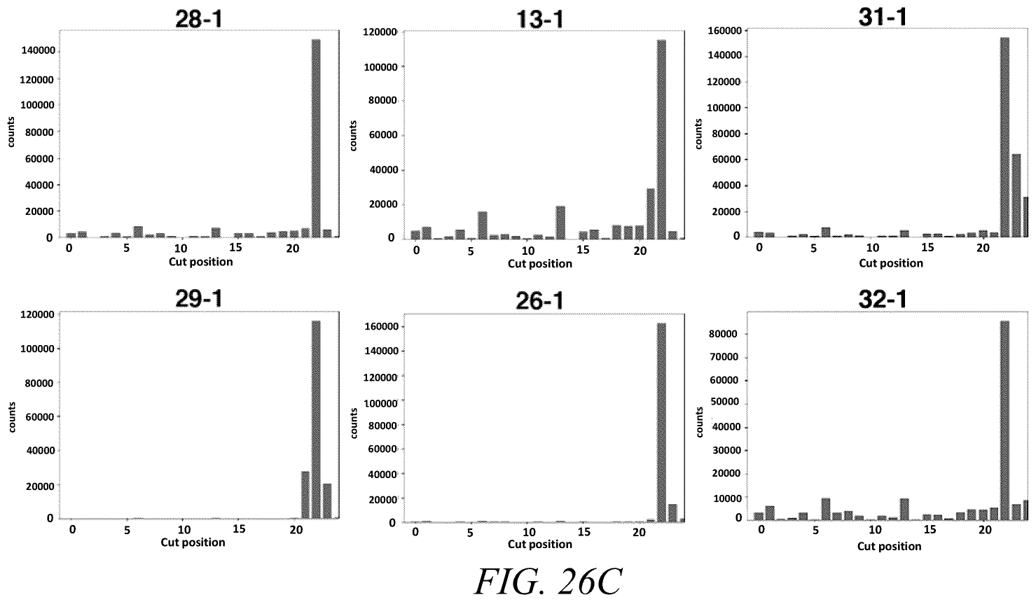

Promising candidates from the bioinformatic analysis and preliminary screens were selected for further biochemical analysis as described in this example. Using the conserved 3′ sgRNA structure, a “universal” sgRNA was designed comprising the 3′ 20 nt of the CRISPR repeat and a 24 nt spacer (FIGS. 10A-D). Of the seven tested candidates, six showed activity in vitro against the 8N PAM library (FIG. 26A). The remaining inactive candidate (30-1) showed activity when tested with its predicted endogenous trimmed CRISPR repeat (SEQ ID NO: 3608, see FIG. 26B), but was not included in NGS library assays. (FIG. 26C)

The majority of identified PAMs are thymine-rich sequences of 2-3 bases (FIG. 18A). However, two enzymes, MG26-1 (PAM YYn) and MG29-1 (PAM YYn), had PAM specificity for either pyrimidine base, thymine or cytosine, allowing for broader sequence targeting. Analysis of putative PAM-interacting residues indicated that the active Type V-A nucleases contain a conserved Lysine and a GWxxxK motif, which were shown to be important in recognition and interaction with different PAMs in FnCas12a.

As our PAM detection assay required ligation to create blunt-end fragments before PAM enrichment, this suggested that these enzymes created a staggered double strand DNA break, similar to reported Type V-A nucleases. The cut site on the target strand can be identified by analysis of the NGS reads used for indel detection (FIG. 18B) and showed cl^{ea}vage after the 22nd PAM-distal base

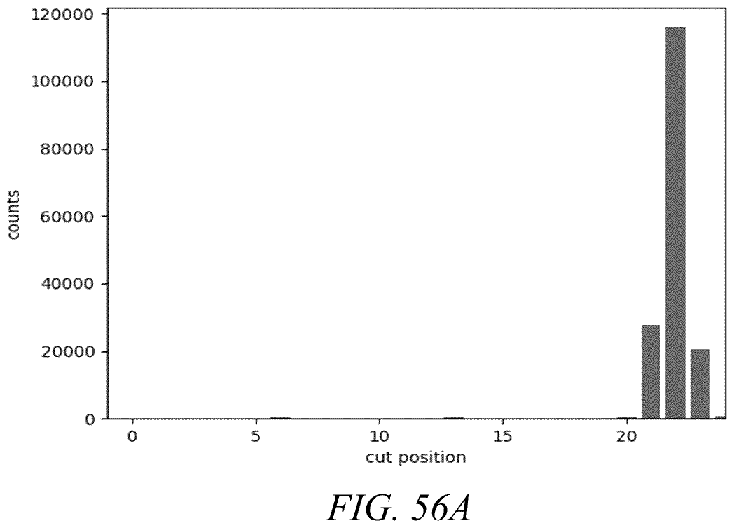

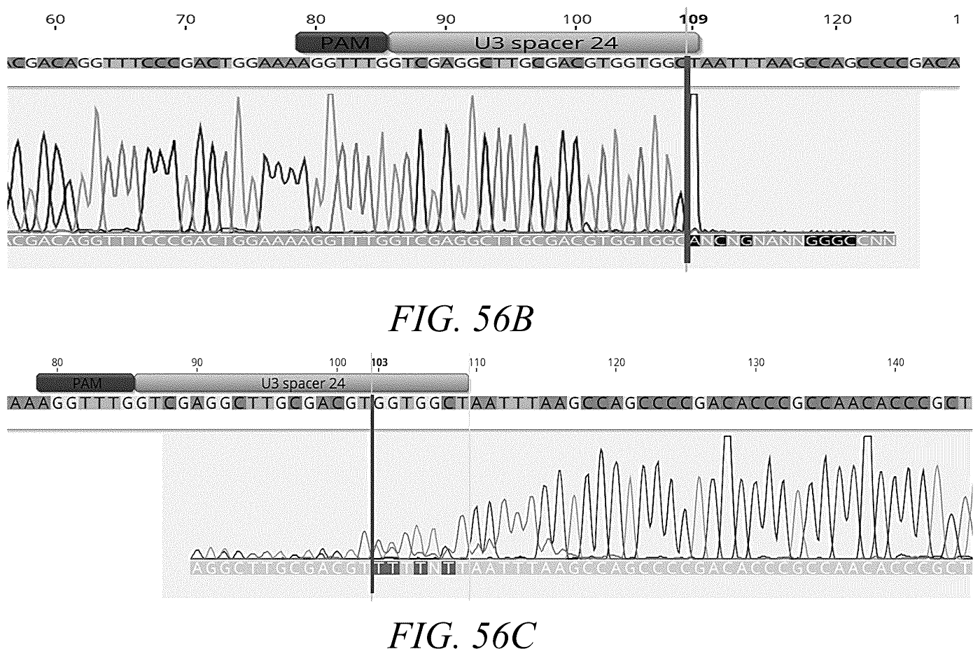

In vitro cleavage by MG29-1 was further investigated by sequencing the cleavage products. The cut position on the target strand was 22 nucleotides away from the PAM in most sequences, and 21 or 23 nucleotides less frequently (FIGS. 56A-C). The cut position on the non-target strand was 17 to 19 nucleotides from the PAM. In combination, these results indicate a 3-5 bp overhang.

### Example 4—Genome Editing

After confirmation of the PAM, novel proteins described herein were tested in HEK293T cells for gene targeting activity. All candidates showed activity of over 5% NHEJ (background corrected) on at least one of ten tested target loci. MG29-1 showed the highest overall activity in NHEJ modification outcomes (FIG. 18B) and was active on the highest number of targets. Thus, this nuclease was selected for purified ribonucleoprotein complex (RNP) testing in HEK293 cells. RNP transfection of MG29-1 holoenzyme showed higher editing levels with RNP than plasmid-based transfection on 4 out of 9 targets, in some cases over 80% editing efficiency (FIG. 18C). Analysis of editing profiles for MG29-1 indicates that this nuclease produces deletions of more than two bp more frequently than other types of edits at their target site (FIG. 18D). At some targets (5 and 8) the indel frequency for MG29-1 was twice that of AsCpf1 (FIG. 18E).

### Example 17—Discussion

Type V-A CRISPR were identified from metagenomes collected from a variety of complex environments and arranged into families. These novel Type V-A nucleases had diverse sequences and phylogenetic origins within and across families and cleaved targets with diverse PAM sites. Similar to other Type V-A nucleases (e.g. LbCas12a, AsCas12a, and FnCas12a), the effectors described herein utilized a single guide CRISPR RNA (sgRNA) to target staggered double stranded cleavage of DNA, simplifying guide design and synthesis, which will facilitate multiplexed editing. Analysis of CRISPR repeat motifs that formed the stem-loop structure of the crRNA suggested that the Type V-A effectors described herein have a 4-nt loop guide more frequently than shorter or longer loops. The sgRNA motif of LbCpf1 has a less common 5-nt, although the 4-nt loop was also observed for 16 Cpf1 orthologs already identified. An unusual stem-loop CRISPR repeat motif sequence, CCUGC[N3.4]GCAGG, was identified for the MG61 family of Type V-A effectors. The high degree of conservation of the sgRNA with variable loop lengths in Type V-A may afford flexible levels of activity, as shown for proteins described herein. Taken together, these effectors are not close homologs to previously studied enzymes, and greatly expand the diversity of Type V-A-like sgRNA nucleases.

Additional Type V effectors described herein may have evolved from duplications of Type V-A-like nucleases, referred to here as Type V-A prime effectors (V-A′) which may be encoded next to Cas12a nucleases. Both Type V-A and these Type V-A′ systems may share a CRISPR sgRNA but the Type V-A′ systems are divergent from Cas12a (FIG. 4). The CRISPR repeat associated with these prime effectors also folded into single guide crRNA with the UCUAC[N₃₋₅]GUAGAU motif. One report identified a Type V cms1 effector encoded next to a Type V-A nuclease, which required a single guide crRNA for cleavage activity in plant cells. Different CRISPR arrays were reported for each effector, while the Type V-A′ system described herein suggested that both Type V-A and V-A′ may require the same crRNA for DNA targeting and cleavage. As described recently in Roizmanbacterial genomes (see e.g., Chen et al. Front Microbiol. 2019 May 3; 10:928), both Type V-A and V-A′ effectors are distantly related based on sequence homology and phylogenetic analysis. Therefore, the prime effectors do not belong within the Type V-A classification, and warrant a separate Type V sub-classification

PAMs determined for active Type V-A nucleases were generally thymine-rich, similar to PAMs described for other Type V-A nucleases. In contrast, MG29-1 requires a shorter YYN PAM sequence, which increases target flexibility compared to the four nucleotide TTTV PAM of LbCpf1. Additionally, RNPs containing MG29-1 had higher activity in HEK293 cells compared to sMbCas12a, which has a three-nucleotide PAM.

When testing the novel nucleases for in-vitro editing activity, MG29-1 exhibited comparable or better activity to other reported enzymes of the class. Reports of plasmid transfection editing efficiencies in mammalian cells using Cas12a orthologs indicate between 21% and 26% indel frequencies for guides with T-rich PAMs, and one out of 18 guides with CCN PAMs showed ˜10% activity in Mb3Cas12a (Moraxella bovoculi AAX11_00205 Cas12a, see e.g. Wang et al. Journal of Cell Science 2020 133: jcs240705). Notably, MG29-1 activity in plasmid transfections appears greater than that reported for Mb3Cas12a for targets with TTN and CCN PAMs (see e.g. FIGS. 18A-E). Because the target sites for plasmid transfections have the same TTG PAM on all experiments, the difference in editing efficiency may be attributed to genomic accessibility differences at different target genes. MG29-1 editing as RNP is much more efficient than via plasmid and is more efficient than AsCas12a on two of seven target loci. Therefore, MG29-1 may be a highly active and efficient gene editing nuclease. These findings increase the diversity of identified single guide Type V-A CRISPR nucleases, and demonstrate the genome editing potential of novel enzymes from uncultivated microbes. Seven novel nucleases showed in-vitro activity with diverse PAM requirements, and RNP data showed editing efficiency surpassing 80% for therapeutically relevant targets in human cell lines. These novel nucleases expand the toolkit of CRISPR-associated enzymes and enable diverse genome engineering applications.

### Example 18—MG29-1 Induced Editing of TRAC Locus in T-Cells

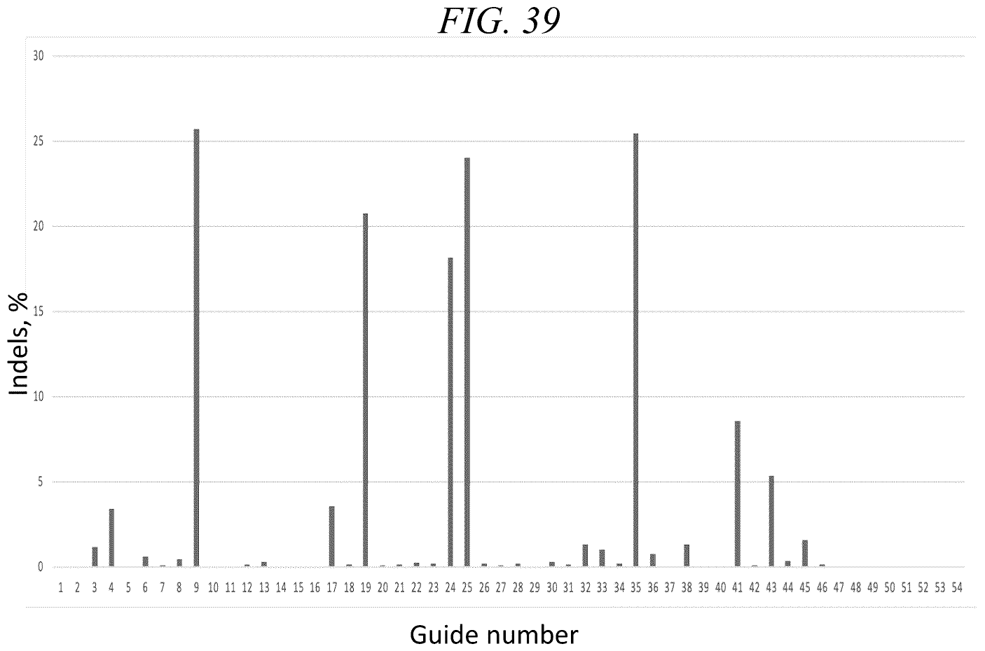

The three exons of the T cell receptor alpha chain constant region (TRACA) were scanned for sequences matching an initial predicted 5′-TTN-3′ PAM specificity of MG29-1 and single-guide RNAs with proprietary Alt-R modifications were ordered from IDT. All guide spacer sequences were 22 nt long. Guides (80 pmol) were mixed with purified MG29-1 protein (63 pmol), incubated for 15 minutes at room temperature. T cells were purified from PBMCs by negative selection using (Stemcell Technologies Human T cell Isolation Kit #17951) and activated by CD2/3/28 beads (Miltenyi T cell Activation/Expansion Kit #130-091-441). After four days of cell growth, each MG29-1/guide RNA mixture was electroporated into 200,000 T cells with a Lonza 4-D Nucleofector, using program EO-115 and P3 buffer. The cells were harvested seventy-two hours post-transfection, genomic DNA was isolated, and PCR amplified for analysis using high-throughput DNA sequencing using primers targeting the TRACA locus. The creation of insertions and deletions characteristic of NHEJ-based gene editing was quantified using a proprietary Python script (see FIG. 39).

TABLE 5A
Guide sequences used in Example 18

| Entity Name | Sequence | SEQ ID NO: |
|---|---|---|
| MG29-1 Guide 1 | ACCGATTTTGATTCTCAAACAA | 4316 |
| Target Sequence | | |
| MG29-1 Guide 2 | TGATTCTCAAACAAATGTGTCA | 4317 |
| Target Sequence | | |
| MG29-1 Guide 3 | GATTCTCAAACAAATGTGTCAC | 4318 |
| Target Sequence | | |
| MG29-1 Guide 4 | ATTCTCAAACAAATGTGTCACA | 4319 |
| Target Sequence | | |
| MG29-1 Guide 5 | TCAAACAAATGTGTCACAAAGT | 4320 |
| Target Sequence | | |
| MG29-1 Guide 6 | TGATGTGTATATCACAGACAAA | 4321 |
| Target Sequence | | |
| MG29-1 Guide 7 | AAGAGCAACAGTGCTGTGGCCT | 4322 |
| Target Sequence | | |
| MG29-1 Guide 8 | GCATGTGCAAACGCCTTCAACA | 4323 |
| Target Sequence | | |
| MG29-1 Guide 9 | CATGTGCAAACGCCTTCAACAA | 4324 |
| Target Sequence | | |
| MG29-1 Guide 10 | AACAACAGCATTATTCCAGAAG | 4325 |
| Target Sequence | | |
| MG29-1 Guide 11 | TTCCAGAAGACACCTTCTTCCC | 4326 |
| Target Sequence | | |
| MG29-1 Guide 12 | CAGAAGACACCTTCTTCCCCAG | 4327 |
| Target Sequence | | |
| MG29-1 Guide 13 | TGGAATAATGCTGTTGTTGAAG | 4328 |
| Target Sequence | | |
| MG29-1 Guide 14 | TTGAAGGCGTTTGCACATGCAA | 4329 |
| Target Sequence | | |
| MG29-1 Guide 15 | AAGGCGTTTGCACATGCAAAGT | 4330 |
| Target Sequence | | |
| MG29-1 Guide 16 | GCACATGCAAAGTCAGATTTGT | 4331 |
| Target Sequence | | |
| MG29-1 Guide 17 | CACATGCAAAGTCAGATTTGTT | 4332 |
| Target Sequence | | |
| MG29-1 Guide 18 | GTTGCTCCAGGCCACAGCACTG | 4333 |
| Target Sequence | | |
| MG29-1 Guide 19 | TTGCTCCAGGCCACAGCACTGT | 4334 |
| Target Sequence | | |
| MG29-1 Guide 20 | CTCCAGGCCACAGCACTGTTGC | 4335 |
| Target Sequence | | |
| MG29-1 Guide 21 | CTCTTGAAGTCCATAGACCTCA | 4336 |
| Target Sequence | | |
| MG29-1 Guide 22 | AAGTCCATAGACCTCATGTCTA | 4337 |
| Target Sequence | | |
| MG29-1 Guide 23 | TGTCTGTGATATACACATCAGA | 4338 |
| Target Sequence | | |
| MG29-1 Guide 24 | GTCTGTGATATACACATCAGAA | 4339 |
| Target Sequence | | |
| MG29-1 Guide 25 | TCTGTGATATACACATCAGAAT | 4340 |
| Target Sequence | | |
| MG29-1 Guide 26 | CTTTGTGACACATTTGTTTGAG | 4341 |
| Target Sequence | | |
| MG29-1 Guide 27 | GTGACACATTTGTTTGAGAATC | 4342 |
| Target Sequence | | |
| MG29-1 Guide 28 | TGACACATTTGTTTGAGAATCA | 4343 |
| Target Sequence | | |
| MG29-1 Guide 29 | GTTTGAGAATCAAAATCGGTGA | 4344 |
| Target Sequence | | |
| MG29-1 Guide 30 | TTTGAGAATCAAAATCGGTGAA | 4345 |
| Target Sequence | | |
| MG29-1 Guide 31 | GAGAATCAAAATCGGTGAATAG | 4346 |
| Target Sequence | | |
| MG29-1 Guide 32 | AGAATCAAAATCGGTGAATAGG | 4347 |
| Target Sequence | | |
| MG29-1 Guide 33 | TCACTGGATTTAGAGTCTCTCA | 4348 |
| Target Sequence | | |
| MG29-1 Guide 34 | AGAGTCTCTCAGCTGGTACACG | 4349 |
| Target Sequence | | |
| MG29-1 Guide 35 | GAGTCTCTCAGCTGGTACACGG | 4350 |
| Target Sequence | | |
| MG29-1 Guide 36 | CTGTGATGTCAAGCTGGTCGAG | 4351 |
| Target Sequence | | |
| MG29-1 Guide 37 | CAAAGCTTTTCTCGACCAGCTT | 4352 |
| Target Sequence | | |
| MG29-1 Guide 38 | AAAGCTTTTCTCGACCAGCTTG | 4353 |
| Target Sequence | | |
| MG29-1 Guide 39 | TCTCGACCAGCTTGACATCACA | 4354 |
| Target Sequence | | |
| MG29-1 Guide 40 | CTCGACCAGCTTGACATCACAG | 4355 |
| Target Sequence | | |
| MG29-1 Guide 41 | TCGACCAGCTTGACATCACAGG | 4356 |
| Target Sequence | | |
| MG29-1 Guide 42 | CAAAACCTGTCAGTGATTGGGT | 4357 |
| Target Sequence | | |
| MG29-1 Guide 43 | AAAACCTGTCAGTGATTGGGTT | 4358 |
| Target Sequence | | |
| MG29-1 Guide 44 | GGTTCCGAATCCTCCTCCTGAA | 4359 |
| Target Sequence | | |
| MG29-1 Guide 45 | CGAATCCTCCTCCTGAAAGTGG | 4360 |
| Target Sequence | | |
| MG29-1 Guide 46 | AATCTGCTCATGACGCTGCGGC | 4361 |
| Target Sequence | | |
| MG29-1 Guide 47 | ATCTGCTCATGACGCTGCGGCT | 4362 |
| Target Sequence | | |
| MG29-1 Guide 48 | AACCCGGCCACTTTCAGGAGGA | 4363 |
| Target Sequence | | |
| MG29-1 Guide 49 | CAGGAGGAGGATTCGGAACCCA | 4364 |
| Target Sequence | | |
| MG29-1 Guide 50 | AGGAGGAGGATTCGGAACCCAA | 4365 |
| Target Sequence | | |
| MG29-1 Guide 51 | GGAACCCAATCACTGACAGGTT | 4366 |
| Target Sequence | | |
| MG29-1 Guide 52 | TGAAAGTTTAGGTTCGTATCTG | 4367 |
| Target Sequence | | |
| MG29-1 Guide 53 | GAAAGTTTAGGTTCGTATCTGT | 4368 |
| Target Sequence | | |
| MG29-1 Guide 54 | AAAGTTTAGGTTCGTATCTGTA | 4369 |
| Target Sequence | | |
| MG29-1 Guide 1 | /AltR1/rUrArArUrUrUrCrUrArCrUrGrUrUrGrUrArGrA | 4370 |
| sgRNA synthesized | rUrArCrCrGrArUrUrUrUrGrArUrUrCrUrCrArArArCr | |
| | ArA/AltR2/ | |
| MG29-1 Guide 2 | /AltR1/rUrArArUrUrUrCrUrArCrUrGrUrUrGrUrArGrA | 4371 |
| sgRNA synthesized | rUrUrGrArUrUrCrUrCrArArArCrArArArUrGrUrGrUr | |
| | CrA/AltR2/ | |
| MG29-1 Guide 3 | /AltR1/rUrArArUrUrUrCrUrArCrUrGrUrUrGrUrArGrA | 4372 |
| sgRNA synthesized | rUrGrArUrUrCrUrCrArArArCrArArArUrGrUrGrUrCr | |
| | ArC/AltR2/ | |
| MG29-1 Guide 4 | /AltR1/rUrArArUrUrUrCrUrArCrUrGrUrUrGrUrArGrA | 4373 |
| sgRNA synthesized | rUrArUrUrCrUrCrArArArCrArArArUrGrUrGrUrCrAr | |
| | CrA/AltR2/ | |
| MG29-1 Guide 5 | /AltR1/rUrArArUrUrUrCrUrArCrUrGrUrUrGrUrArGrA | 4374 |
| sgRNA synthesized | rUrUrCrArArArCrArArArUrGrUrGrUrCrArCrArArAr | |
| | GrU/AltR2/ | |
| MG29-1 Guide 6 | /AltR1/rUrArArUrUrUrCrUrArCrUrGrUrUrGrUrArGrA | 4375 |
| sgRNA synthesized | rUrUrGrArUrGrUrGrUrArUrArUrCrArCrArGrArCrAr | |
| | ArA/AltR2/ | |
| MG29-1 Guide 7 | /AltR1/rUrArArUrUrUrCrUrArCrUrGrUrUrGrUrArGrA | 4376 |
| sgRNA synthesized | rUrArArGrArGrCrArArCrArGrUrGrCrUrGrUrGrGrCr | |
| | CrU/AltR2/ | |
| MG29-1 Guide 8 | /AltR1/rUrArArUrUrUrCrUrArCrUrGrUrUrGrUrArGrA | 4377 |
| sgRNA synthesized | rUrGrCrArUrGrUrGrCrArArArCrGrCrCrUrUrCrArAr | |
| | CrA/AltR2/ | |
| MG29-1 Guide 9 | /AltR1/rUrArArUrUrUrCrUrArCrUrGrUrUrGrUrArGrA | 4378 |
| sgRNA synthesized | rUrCrArUrGrUrGrCrArArArCrGrCrCrUrUrCrArArCr | |
| | ArA/AltR2/ | |
| MG29-1 Guide 10 | /AltR1/rUrArArUrUrUrCrUrArCrUrGrUrUrGrUrArGrA | 4379 |
| sgRNA synthesized | rUrArArCrArArCrArGrCrArUrUrArUrUrCrCrArGrAr | |
| | ArG/AltR2/ | |
| MG29-1 Guide 11 | /AltR1/rUrArArUrUrUrCrUrArCrUrGrUrUrGrUrArGrA | 4380 |
| sgRNA synthesized | rUrUrUrCrCrArGrArArGrArCrArCrCrUrUrCrUrUrCr | |
| | CrC/AltR2/ | |
| MG29-1 Guide 12 | /AltR1/rUrArArUrUrUrCrUrArCrUrGrUrUrGrUrArGrA | 4381 |
| sgRNA synthesized | rUrCrArGrArArGrArCrArCrCrUrUrCrUrUrCrCrCrCr | |
| | ArG/AltR2/ | |
| MG29-1 Guide 13 | /AltR1/rUrArArUrUrUrCrUrArCrUrGrUrUrGrUrArGrA | 4382 |
| sgRNA synthesized | rUrUrGrGrArArUrArArUrGrCrUrGrUrUrGrUrUrGrAr | |
| | ArG/AltR2/ | |
| MG29-1 Guide 14 | /AltR1/rUrArArUrUrUrCrUrArCrUrGrUrUrGrUrArGrA | 4383 |
| sgRNA synthesized | rUrUrUrGrArArGrGrCrGrUrUrUrGrCrArCrArUrGrCr | |
| | ArA/AltR2/ | |
| MG29-1 Guide 15 | /AltR1/rUrArArUrUrUrCrUrArCrUrGrUrUrGrUrArGrA | 4384 |
| sgRNA synthesized | rUrArArGrGrCrGrUrUrUrGrCrArCrArUrGrCrArArAr | |
| | GrU/AltR2/ | |
| MG29-1 Guide 16 | /AltR1/rUrArArUrUrUrCrUrArCrUrGrUrUrGrUrArGrA | 4385 |
| sgRNA synthesized | rUrGrCrArCrArUrGrCrArArArGrUrCrArGrArUrUrUr | |
| | GrU/AltR2/ | |
| MG29-1 Guide 17 | /AltR1/rUrArArUrUrUrCrUrArCrUrGrUrUrGrUrArGrA | 4386 |
| sgRNA synthesized | rUrCrArCrArUrGrCrArArArGrUrCrArGrArUrUrUrGr | |
| | UrU/AltR2/ | |
| MG29-1 Guide 18 | /AltR1/rUrArArUrUrUrCrUrArCrUrGrUrUrGrUrArGrA | 4387 |
| sgRNA synthesized | rUrGrUrUrGrCrUrCrCrArGrGrCrCrArCrArGrCrArCr | |
| | UrG/AltR2/ | |
| MG29-1 Guide 19 | /AltR1/rUrArArUrUrUrCrUrArCrUrGrUrUrGrUrArGrA | 4388 |
| sgRNA synthesized | rUrUrUrGrCrUrCrCrArGrGrCrCrArCrArGrCrArCrUr | |
| | GrU/AltR2/ | |
| MG29-1 Guide 20 | /AltR1/rUrArArUrUrUrCrUrArCrUrGrUrUrGrUrArGrA | 4389 |
| sgRNA synthesized | rUrCrUrCrCrArGrGrCrCrArCrArGrCrArCrUrGrUrUr | |
| | GrC/AltR2/ | |
| MG29-1 Guide 21 | /AltR1/rUrArArUrUrUrCrUrArCrUrGrUrUrGrUrArGrA | 4390 |
| sgRNA synthesized | rUrCrUrCrUrUrGrArArGrUrCrCrArUrArGrArCrCrUr | |
| | CrA/AltR2/ | |
| MG29-1 Guide 22 | /AltR1/rUrArArUrUrUrCrUrArCrUrGrUrUrGrUrArGrA | 4391 |
| sgRNA synthesized | rUrArArGrUrCrCrArUrArGrArCrCrUrCrArUrGrUrCr | |
| | UrA/AltR2/ | |
| MG29-1 Guide 23 | /AltR1/rUrArArUrUrUrCrUrArCrUrGrUrUrGrUrArGrA | 4392 |
| sgRNA synthesized | rUrUrGrUrCrUrGrUrGrArUrArUrArCrArCrArUrCrAr | |
| | GrA/AltR2/ | |
| MG29-1 Guide 24 | /AltR1/rUrArArUrUrUrCrUrArCrUrGrUrUrGrUrArGrA | 4393 |
| sgRNA synthesized | rUrGrUrCrUrGrUrGrArUrArUrArCrArCrArUrCrArGr | |
| | ArA/AltR2/ | |
| MG29-1 Guide 25 | /AltR1/rUrArArUrUrUrCrUrArCrUrGrUrUrGrUrArGrA | 4394 |
| sgRNA synthesized | rUrUrCrUrGrUrGrArUrArUrArCrArCrArUrCrArGrAr | |
| | ArU/AltR2/ | |
| MG29-1 Guide 26 | /AltR1/rUrArArUrUrUrCrUrArCrUrGrUrUrGrUrArGrA | 4395 |
| sgRNA synthesized | rUrCrUrUrUrGrUrGrArCrArCrArUrUrUrGrUrUrUrGr | |
| | ArG/AltR2/ | |
| MG29-1 Guide 27 | /AltR1/rUrArArUrUrUrCrUrArCrUrGrUrUrGrUrArGrA | 4396 |
| sgRNA synthesized | rUrGrUrGrArCrArCrArUrUrUrGrUrUrUrGrArGrArAr | |
| | UrC/AltR2/ | |
| MG29-1 Guide 28 | /AltR1/rUrArArUrUrUrCrUrArCrUrGrUrUrGrUrArGrA | 4397 |
| sgRNA synthesized | rUrUrGrArCrArCrArUrUrUrGrUrUrUrGrArGrArArUr | |
| | CrA/AltR2/ | |
| MG29-1 Guide 29 | /AltR1/rUrArArUrUrUrCrUrArCrUrGrUrUrGrUrArGrA | 4398 |
| sgRNA synthesized | rUrGrUrUrUrGrArGrArArUrCrArArArArUrCrGrGrUr | |
| | GrA/AltR2/ | |
| MG29-1 Guide 30 | /AltR1/rUrArArUrUrUrCrUrArCrUrGrUrUrGrUrArGrA | 4399 |
| sgRNA synthesized | rUrUrUrUrGrArGrArArUrCrArArArArUrCrGrGrUrGr | |
| | ArA/AltR2/ | |
| MG29-1 Guide 31 | /AltR1/rUrArArUrUrUrCrUrArCrUrGrUrUrGrUrArGrA | 4400 |
| sgRNA synthesized | rUrGrArGrArArUrCrArArArArUrCrGrGrUrGrArArUr | |
| | ArG/AltR2/ | |
| MG29-1 Guide 32 | /AltR1/rUrArArUrUrUrCrUrArCrUrGrUrUrGrUrArGrA | 4401 |
| sgRNA synthesized | rUrArGrArArUrCrArArArArUrCrGrGrUrGrArArUrAr | |
| | GrG/AltR2/ | |
| MG29-1 Guide 33 | /AltR1/rUrArArUrUrUrCrUrArCrUrGrUrUrGrUrArGrA | 4402 |
| sgRNA synthesized | rUrUrCrArCrUrGrGrArUrUrUrArGrArGrUrCrUrCrUr | |
| | CrA/AltR2/ | |
| MG29-1 Guide 34 | /AltR1/rUrArArUrUrUrCrUrArCrUrGrUrUrGrUrArGrA | 4403 |
| sgRNA synthesized | rUrArGrArGrUrCrUrCrUrCrArGrCrUrGrGrUrArCrAr | |
| | CrG/AltR2/ | |
| MG29-1 Guide 35 | /AltR1/rUrArArUrUrUrCrUrArCrUrGrUrUrGrUrArGrA | 4404 |
| sgRNA synthesized | rUrGrArGrUrCrUrCrUrCrArGrCrUrGrGrUrArCrArCr | |
| | GrG/AltR2/ | |
| MG29-1 Guide 36 | /AltR1/rUrArArUrUrUrCrUrArCrUrGrUrUrGrUrArGrA | 4405 |
| sgRNA synthesized | rUrCrUrGrUrGrArUrGrUrCrArArGrCrUrGrGrUrCrGr | |
| | ArG/AltR2/ | |
| MG29-1 Guide 37 | /AltR1/rUrArArUrUrUrCrUrArCrUrGrUrUrGrUrArGrA | 4406 |
| sgRNA synthesized | rUrCrArArArGrCrUrUrUrUrCrUrCrGrArCrCrArGrCr | |
| | UrU/AltR2/ | |
| MG29-1 Guide 38 | /AltR1/rUrArArUrUrUrCrUrArCrUrGrUrUrGrUrArGrA | 4407 |
| sgRNA synthesized | rUrArArArGrCrUrUrUrUrCrUrCrGrArCrCrArGrCrUr | |
| | UrG/AltR2/ | |
| MG29-1 Guide 39 | /AltR1/rUrArArUrUrUrCrUrArCrUrGrUrUrGrUrArGrA | 4408 |
| sgRNA synthesized | rUrUrCrUrCrGrArCrCrArGrCrUrUrGrArCrArUrCrAr | |
| | CrA/AltR2/ | |
| MG29-1 Guide 40 | /AltR1/rUrArArUrUrUrCrUrArCrUrGrUrUrGrUrArGrA | 4409 |
| sgRNA synthesized | rUrCrUrCrGrArCrCrArGrCrUrUrGrArCrArUrCrArCr | |
| | ArG/AltR2/ | |
| MG29-1 Guide 41 | /AltR1/rUrArArUrUrUrCrUrArCrUrGrUrUrGrUrArGrA | 4410 |
| sgRNA synthesized | rUrUrCrGrArCrCrArGrCrUrUrGrArCrArUrCrArCrAr | |
| | GrG/AltR2/ | |
| MG29-1 Guide 42 | /AltR1/rUrArArUrUrUrCrUrArCrUrGrUrUrGrUrArGrA | 4411 |
| sgRNA synthesized | rUrCrArArArArCrCrUrGrUrCrArGrUrGrArUrUrGrGr | |
| | GrU/AltR2/ | |
| MG29-1 Guide 43 | /AltR1/rUrArArUrUrUrCrUrArCrUrGrUrUrGrUrArGrA | 4412 |
| sgRNA synthesized | rUrArArArArCrCrUrGrUrCrArGrUrGrArUrUrGrGrGr | |
| | UrU/AltR2/ | |
| MG29-1 Guide 44 | /AltR1/rUrArArUrUrUrCrUrArCrUrGrUrUrGrUrArGrA | 4413 |
| sgRNA synthesized | rUrGrGrUrUrCrCrGrArArUrCrCrUrCrCrUrCrCrUrGr | |
| | ArA/AltR2/ | |
| MG29-1 Guide 45 | /AltR1/rUrArArUrUrUrCrUrArCrUrGrUrUrGrUrArGrA | 4414 |
| sgRNA synthesized | rUrCrGrArArUrCrCrUrCrCrUrCrCrUrGrArArArGrUr | |
| | GrG/AltR2/ | |
| MG29-1 Guide 46 | /AltR1/rUrArArUrUrUrCrUrArCrUrGrUrUrGrUrArGrA | 4415 |
| sgRNA synthesized | rUrArArUrCrUrGrCrUrCrArUrGrArCrGrCrUrGrCrGr | |
| | GrC/AltR2/ | |
| MG29-1 Guide 47 | /AltR1/rUrArArUrUrUrCrUrArCrUrGrUrUrGrUrArGrA | 4416 |
| sgRNA synthesized | rUrArUrCrUrGrCrUrCrArUrGrArCrGrCrUrGrCrGrGr | |
| | CrU/AltR2/ | |
| MG29-1 Guide 48 | /AltR1/rUrArArUrUrUrCrUrArCrUrGrUrUrGrUrArGrA | 4417 |
| sgRNA synthesized | rUrArArCrCrCrGrGrCrCrArCrUrUrUrCrArGrGrArGr | |
| | GrA/AltR2/ | |
| MG29-1 Guide 49 | /AltR1/rUrArArUrUrUrCrUrArCrUrGrUrUrGrUrArGrA | 4418 |
| sgRNA synthesized | rUrCrArGrGrArGrGrArGrGrArUrUrCrGrGrArArCrCr | |
| | CrA/AltR2/ | |
| MG29-1 Guide 50 | /AltR1/rUrArArUrUrUrCrUrArCrUrGrUrUrGrUrArGrA | 4419 |
| sgRNA synthesized | rUrArGrGrArGrGrArGrGrArUrUrCrGrGrArArCrCrCr | |
| | ArA/AltR2/ | |
| MG29-1 Guide 51 | /AltR1/rUrArArUrUrUrCrUrArCrUrGrUrUrGrUrArGrA | 4420 |
| sgRNA synthesized | rUrGrGrArArCrCrCrArArUrCrArCrUrGrArCrArGrGr | |
| | UrU/AltR2/ | |
| MG29-1 Guide 52 | /AltR1/rUrArArUrUrUrCrUrArCrUrGrUrUrGrUrArGrA | 4421 |
| sgRNA synthesized | rUrUrGrArArArGrUrUrUrArGrGrUrUrCrGrUrArUrCr | |
| | UrG/AltR2/ | |
| MG29-1 Guide 53 | /AltR1/rUrArArUrUrUrCrUrArCrUrGrUrUrGrUrArGrA | 4422 |
| sgRNA synthesized | rUrGrArArArGrUrUrUrArGrGrUrUrCrGrUrArUrCrUr | |
| | GrU/AltR2/ | |
| MG29-1 Guide 54 | /AltR1/rUrArArUrUrUrCrUrArCrUrGrUrUrGrUrArGrA | 4423 |
| sgRNA synthesized | rUrArArArGrUrUrUrArGrGrUrUrCrGrUrArUrCrUrGr | |
| | UrA/AltR2/ | |

### Example 19—Re-Testing of Lead Guides of MG29-1

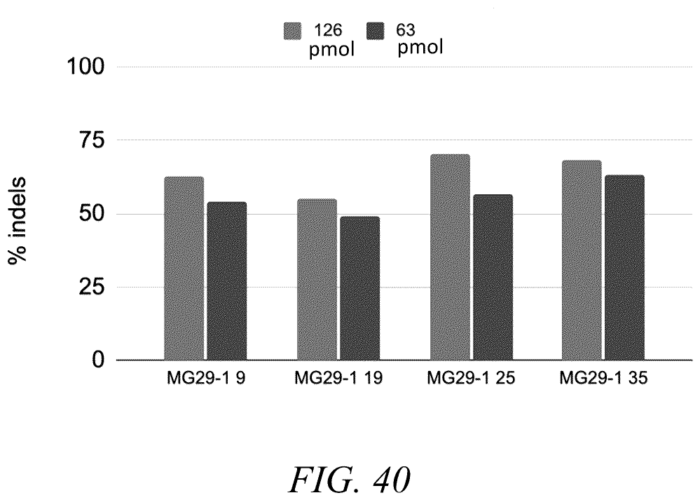

An experiment retesting the lead guides for MG29-1 was performed. The three exons of the T cell receptor alpha chain constant region were scanned for sequences matching 5′-TTN-3′ and single-guide RNAs ordered from IDT using Alt-R modifications. All guide spacer sequences 22 nt long. Guides were mixed with purified MG29-1 protein (80 pmol gRNA+63 pmol MG29-1; or 160 pmol gRNA with 126 pmol MG29-1), incubated for 15 minutes at room temperature. T cells were purified from PBMCs by negative selection using (Stemcell Technologies Human T cell Isolation Kit #17951) and activated by CD2/3/28 beads (Miltenyi T cell Activation/Expansion Kit #130-091-441). After four days of cell growth, each MG29-1/guide RNA mixture was electroporated into 200,000 T cells with a Lonza 4-D Nucleofector, using program EO-115 and P3 buffer. Seventy-two hours post-transfection, genomic DNA was harvested, and PCR amplified for analysis using high-throughput DNA sequencing. The creation of insertions and deletions characteristic of NHEJ-based gene editing was quantified using a proprietary Python script (see FIG. 40).

### Example 20—Testing Length of Guide Spacer for MG29-1

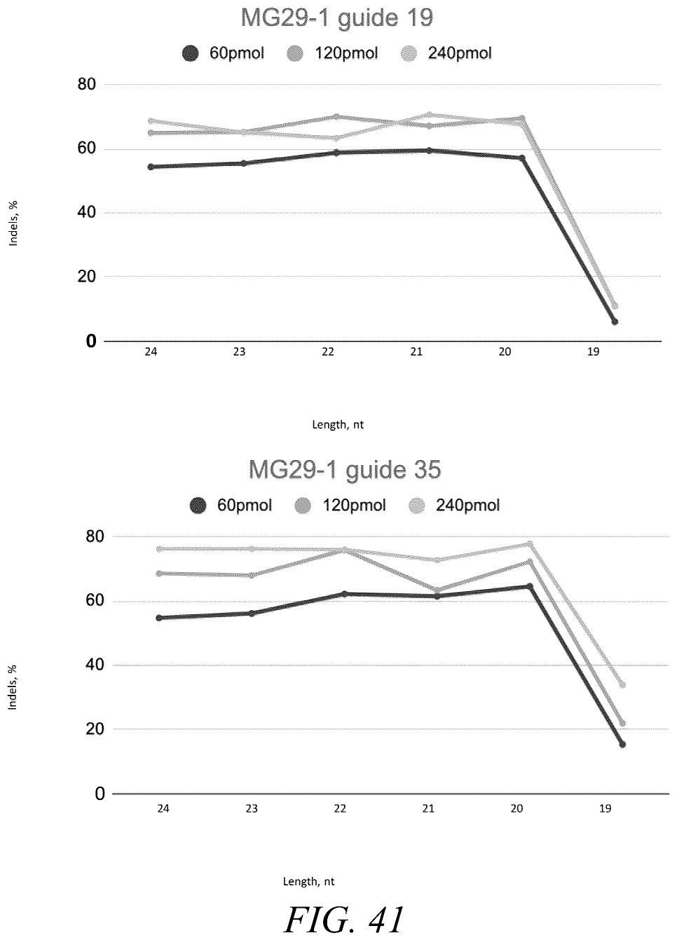

An experiment was performed to determine the optimal guide spacer length. The three exons of the T cell receptor alpha chain constant region were scanned for sequences matching 5′-TTN-3′ and single-guide RNAs ordered from IDT using Alt-R modifications. Guides were mixed with purified MG29-1 protein (80 pmol gRNA+60 pmol effector; 160 pmol gRNA+120 pmol effector; or 320 pmol gRNA+240 pmol effector), incubated for 15 minutes at room temperature. T cells were purified from PBMCs by negative selection using (Stemcell Technologies Human T cell Isolation Kit #17951) and activated by CD2/3/28 beads (Miltenyi T cell Activation/Expansion Kit #130-091-441). After four days of cell growth, each MG29-1/guide RNA mixture was electroporated into 200,000 T cells with a Lonza 4-D Nucleofector, using program EO-115 and P3 buffer. Seventy-two hours post-transfection, genomic DNA was harvested, and PCR amplified for analysis using high-throughput DNA sequencing. The creation of insertions and deletions characteristic of NHEJ-based gene editing was quantified using a proprietary Python script. The results are shown in FIG. 41, which demonstrates that guide spacer lengths of 20-24 nt work well, with a dropoff at 19 nt.

### Example 21—Determination of MG29-1 Indel Generation Versus TCR Expression

Cells from FIG. 41 were analyzed for TCR expression by flow cytometry using the APC-labeled anti-human TCRα/β Ab (Biolegend #306718, clone IP26) and an Attune NxT flow cytometer (Thermo Fisher). Indel data are taken from FIG. 41.

### Example 22—Targeted CAR Integration with MG29-1

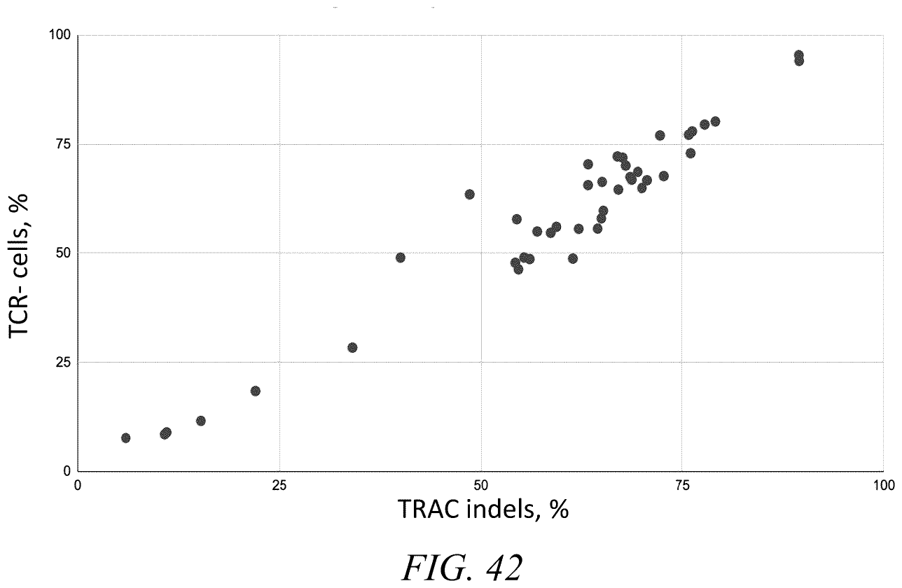

The three exons of the T cell receptor alpha chain constant region were scanned for sequences matching 5′-TTN-3′ and single-guide RNAs ordered from IDT using IDT's proprietary Alt-R modifications. Guides (80 pmol) were mixed with purified MG29-1 protein (63 pmol), incubated for 15 minutes at room temperature. T cells were purified from PBMCs by negative selection using (Stemcell Technologies Human T cell Isolation Kit #17951) and activated by CD2/3/28 beads (Miltenyi T cell Activation/Expansion Kit #130-091-441). After four days of cell growth, each MG29-1/guide RNA mixture was electroporated into 200,000 T cells with a Lonza 4-D Nucleofector, using program EO-115 and P3 buffer. 100,000 vector genomes of a serotype 6 adeno-associated virus (AAV-6) containing the coding sequence for a customized chimeric antigen receptor flanked by 5′ and 3′ homology arms (5′ arm SEQ ID NO: 4424 being about 500 nt in length and 3′ arm SEQ ID NO: 4425 being about 500 nt in length) targeting the TRAC gene were added to the cells immediately following transfection. Replicates were analyzed for TCR expression versus TRAC indels (FIG. 42), showing that indels in the TRAC gene correlated with loss of expression of TCR Cells were also analyzed by flow cytometry simultaneously for TCR expression as in Example 21 (FIG. 42) and for binding of the target antigen to the CAR (FIG. 43, in which the plots are gated on single, live cells). The results of the flow analysis in FIG. 43 indicated that while the guide RNAs alone were effective in eliminating TCR expression (“RNP only”), addition of guide RNA plus AAV resulted in a new population of cells binding the CAR antigen (top left of plots “AAV+MG29-1-19-22” and “AAV+MG29-1-35-22”). The sgRNA 35 (SEQ ID NO: 4404) was somewhat more effective in inducing integration of the CAR than sgRNA 19 (SEQ ID NO: 4388). One possible explanation for the difference is that the predicted nuclease cut site for Guide 19 is −160 bp away from the end of the right homology arm.

TABLE 5B
Guide RNAs used in Example 22

| Entity Name | Sequence | SEQ ID NO: |
|---|---|---|
| MG29-1 TRAC | TTGCTCCAGGCCACAGCACTGT | 4334 |
| Guide 19 | | |
| Target- | | |
| binding | | |
| Sequence | | |
| MG29-1 TRAC | /AltR1/rUrArArUrUrUrCr | 4388 |
| Guide 19 | UrArCrUrGrUrUrGrUrArGr | |
| full sgRNA | ArUrUrUrGrCrUrCrCrArGr | |
| synthesized | GrCrCrArCrArGrCrArCrUr | |
| | GrU/AltR2/ | |
| MG29-1 | GAGTCTCTCAGCTGGTACACGG | 4350 |
| TRAC Guide | | |
| 35 Target- | | |
| binding | | |
| Sequence | | |
| MG29-1 TRAC | /AltR1/rUrArArUrUrUrCrUr | 4404 |
| Guide 35 | ArCrUrGrUrUrGrUrArGrArUr | |
| full sgRNA | GrArGrUrCrUrCrUrCrArGrCr | |
| synthesized | UrGrGrUrArCrArCrGrG/AltR2/ | |

/AltR1/ and /AltR2/ refer to IDT's proprietary Alt-R 5′ and 3′ modifications; m; 2′-O-methyl base (for example an A base with 2′-O-methyl modification is written as mA), i2F; internal 2′-flourine base (for example an internal C with 2′-flourine modification is written as /12FC/), 52F; 2′-flourine base at the 5′ end of the sequence (for example a 5′ C with 2′-flourine modification is written as /52FC/), 32F; 2′-flourine base at the 3′ end of the sequence (for example a 3′ A base with 2′-flourine modification is written as /32FA/), r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur

### Example 5—MG29-1 TRAC Editing in HSCs

Hematopoietic stem cells were purchased from Allcells and thawed per the supplier's instructions, washed in DMEM+10% FBS, and resuspended in Stemspan II medium plus CC110 cytokines. One million cells were cultured for 72 hours in a 6-well dish in 4 mL medium. MG29-1 RNPs were made, transfected, and gene editing analyzed as in Example 18 except for use of the EO-100 nucleofection program. The results are shown in FIG. 61, which shows gene editing at TRAC in hematopoietic stem cells using the #19 (SEQ ID NO:4388) and #35 (SEQ ID NO: 4404) sgRNAs in Table 5B below. The results again indicate that the #35 sgRNA is highly effective at targeting the TRAC locus.

TABLE 5C
Guide RNAs used in Example 23

| | | SEQ |
|---|---|---|
| | | ID |
| Entity Name | Sequence | NO: |
| MG29-1 | TTGCTCCAGGCCACAGCACTGT | 4334 |
| TRAC Guide | | |
| 19 Target- | | |
| binding | | |
| Sequence | | |
| MG29-1 TRAC | /AltR1/rUrArArUrUrUrCr | 4388 |
| Guide 19 | UrArCrUrGrUrUrGrUrArGr | |
| full sgRNA | ArUrUrUrGrCrUrCrCrArGr | |
| synthesized | GrCrCrArCrArGrCrArCrUr | |
| | GrU/AltR2/ | |
| MG29-1 | GAGTCTCTCAGCTGGTACACGG | 4350 |
| TRAC Guide | | |
| 35 Target- | | |
| binding | | |
| Sequence | | |
| MG29-1 | /AltR1/rUrArArUrUrUrCr | 4404 |
| TRAC Guide | UrArCrUrGrUrUrGrUrArGr | |
| 35 | ArUrGrArGrUrCrUrCrUrCr | |
| full sgRNA | ArGrCrUrGrGrUrArCrArCr | |
| synthesized | GrG/AltR2/ | |

/AltR1/ and /AltR2/ refer to IDT's proprietary Alt-R 5′ and 3′ modifications; m; 2′-O-methyl base (for example a A base with 2′-O-methyl modification is written as mA), i2F; internal 2′-flourine base (for example an internal C with 2′-flourine modification is written as /i2FC/), 52F; 2′-flourine base at the 5′ end of the sequence (for example a 5′ C with 2′-flourine modification is written as /52FC/), 32F; 2′-flourine base at the 3′ end of the sequence (for example a 3′ A base with 2′-flourine modification is written as /32FA/), r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur

### Example 6—Further Analysis of PAM Specificity Associated with MG29-1

Further analysis was performed to determine more precisely the PAM specificity of MG29-1. Guide RNAs were designed using a 5′-NTTN-3′ PAM sequence and then sorted according to the gene editing activity observed (FIG. 45, in which the identity of the underlined base—the 5′-proximal N is shown for each bin). All of the guides with activity greater than 10% had a T at this position in the genomic DNA indicating that the MG29-1 PAM may be better described as 5′-TTTN-3′. The statistical significance of the over-representation of T at this position is shown for each bin. In FIG. 45, the various bins (High, medium, low, >1%, <1%) signify:

$$High : > 50 \% indels ( N = 4 )$$

$$Medium : 10 - 50 \% indels ( N = 15 )$$

$$Low : 5 - 10 \% indels ( N = 5 )$$

$$> 1 \% : 1 - 5 \% indels ( N = 12 )$$

$$< 1 \% ( N = 82 )$$

TABLE 2
p-values for nucleotide specificity analysis in Example 24

| | chi\hat{ }2 p-value |
|---|---|
| high/med | 0.000005 |
| low | 0.035110 |
| >1% | 0.005416 |
| <1% | 0.126751 |

### Example 7—Determining MG29-1 Indel Induction Ability Vs Spacer Base Composition

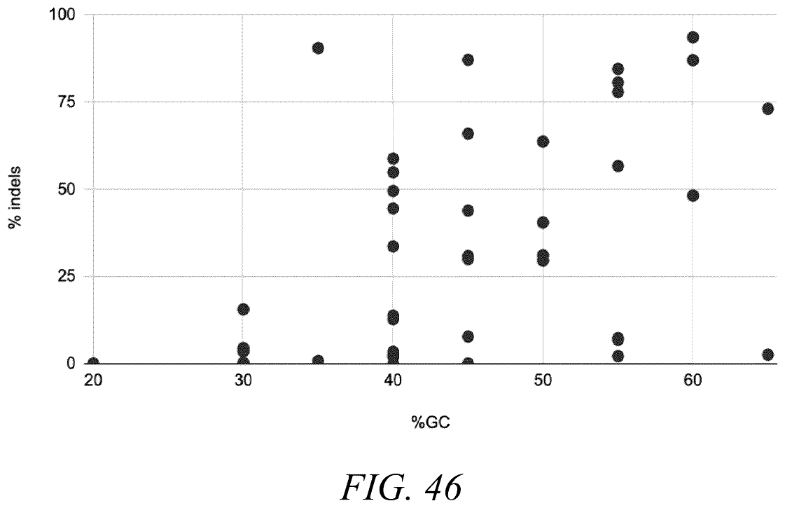

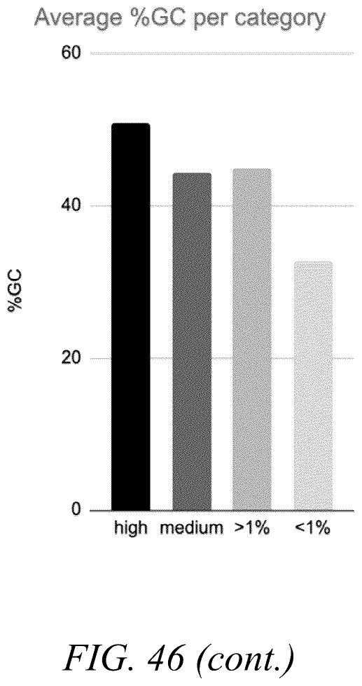

Further analysis was conducted of gene editing activity versus the base composition of MG29-1 spacer sequences. The correlation was modest (R\hat{ }=0.23) but there is a trend towards better activity with higher GC content (see FIG. 46, in which correlation between indels induced in cultured cells versus GC content of spacer sequences is presented as a dot plot).

### Example 26—MG29-1 Guide Chemistry Modifications

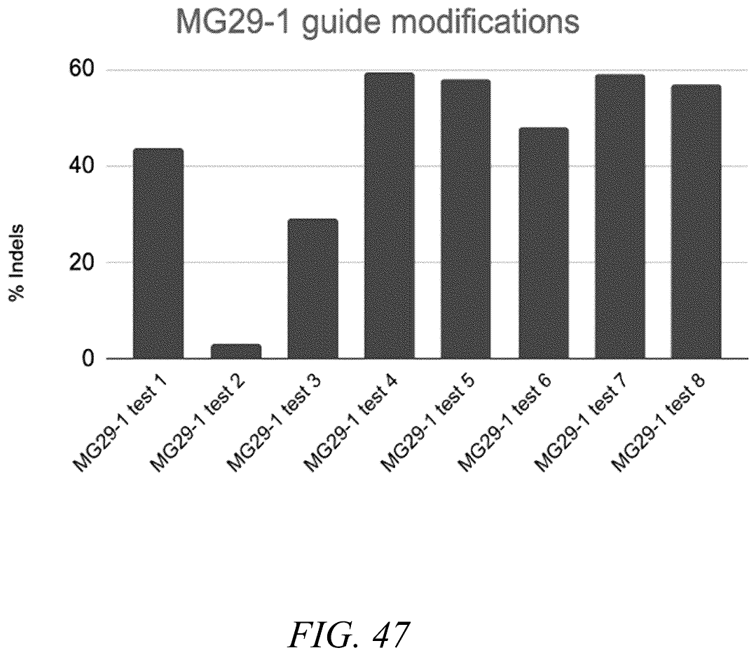

An experiment to optimize chemical modifications for targeting of the VEGF-A locus using MG29-1 was performed, using the procedure of Example 18 but with the indicated guide RNAs targeting VEGF-A (see Table 7 below). The experiment used 126 pmol MG29-1 and 160 pmol guide RNA. The results are presented in FIG. 47. Guides #4, 5, 6, 7, and 8 showed improved activity versus the unmodified guide #1, indicating that the corresponding modifications in these sequences improved the activity of these guide RNAs versus an unmodified RNA sequence.

TABLE 7
MG29-1 guide modifications

| | MG29-1 guide with targeting | |
|---|---|---|
| MG29- | sequence in bold and | SEQ |
| 1 Test | modifications per | ID |
| No. | legend below | NO: |
| 1 | UAAUUUCUACUCUUGUAGAU | 3985 |
| | GAAAGGGGGTGGGGGGAGTT | |
| | TGCT | |
| 2 | mU*mA*mA*UUUCUACUCUU | 3986 |
| | GUAGAUGAAAGGGGGTGGGG | |
| | GGAGTTT*mG*mC*mT | |
| 3 | mU*mA*AUUUCUACUCUUGU | 3987 |
| | AGAUGAAAGGGGGTGGGGGG | |
| | AGTTT*mG*mC*mT | |
| 4 | mU*AAUUUCUACUCUUGUAG | 3988 |
| | AUGAAAGGGGGTGGGGGGAG | |
| | TTT*mG*mC*mT | |
| 5 | mU*AAUUUCUACUCUUGUAG | 3989 |
| | AUGAAAGGGGGTGGGGGGAG | |
| | TTTGC*mT | |
| 6 | mC*UAAUUUCUACUCUUGUA | 3990 |
| | GAUGAAAGGGGGTGGGGGGA | |
| | GTTT*mG*mC*mT | |
| 7 | mC*U*AAUUUCUACUCUUGU | 3991 |
| | AGAUGAAAGGGGGTGGGGGG | |
| | AGTTTG*C*mT | |
| 8 | /AltR1/UAAUUUCUACUCU | 3992 |
| | UGUAGAUGAAAGGGGGTGGG | |
| | GGGAGTTTGCT/AltR2/ | |

Legend:
/AltR1/ and /AltR2/ refer to IDT's proprietary Alt-R 5′ and 3′ modifications; m; 2′-O-methyl base (for example a base with 2′-O-methyl modification is written as mA), i2F; internal 2′-flourine base (for example an internal C with 2′-flourine modification is written as /12FC/), 52F; 2′-flourine base at the 5′ end of the sequence (for example a 5′ C with 2′-flourine modification is written as /52FC/), 32F; 2′-flourine base at the 3′ end of the sequence (for example a 3′ A base with 2′-flourine modification is written as /32FA/), r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur

### Example 27—Titration of Modified MG29-1 Guides from Example 26

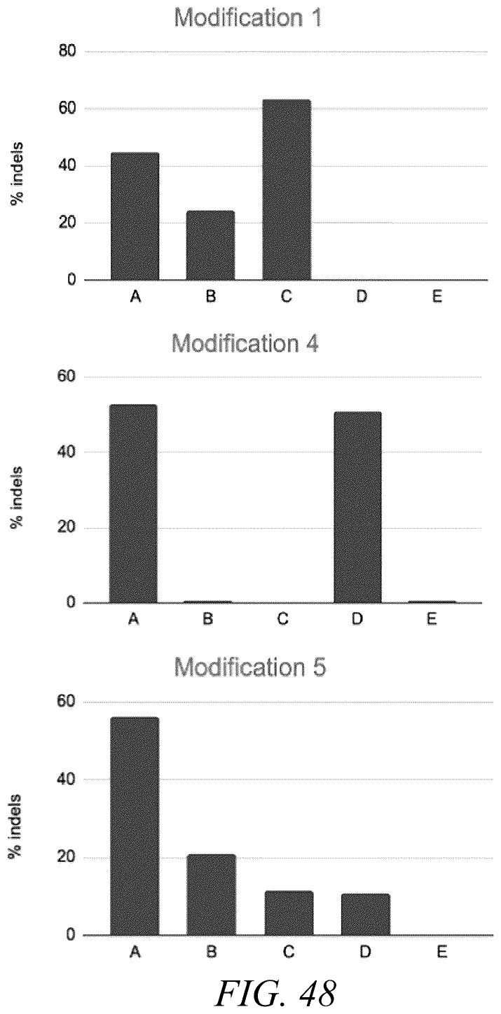

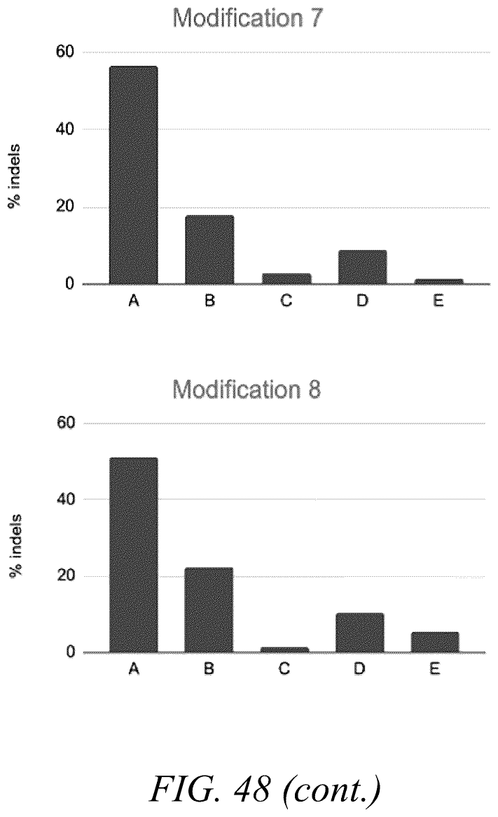

A further experiment was performed to determine the dose dependence of the activity of the modified guides used in Example 26 to identify possible dose-dependent toxicity effects. The experiment was performed as in Example 26 but with ¼th (B), ⅛th (C), 1/16th (D), and 1/32nd (E) of the starting dose (A, 126 pmol MG29-1 and 160 pmol guide RNA). The results are presented in FIG. 48).

### Example 28—Large Scale Synthesis of Nucleases Described Herein

### Project Overview

Production of Metagenomi's Type V-A CRISPR nuclease, MG29-1, is scaled up to an initial culture volume of 10 L. An expression screen, scaled-up expression, downstream development, a formulation study, and delivery of purified protein >=90% by SDS-PAGE are performed.

### Expression and Purfication Screen

### Expression:

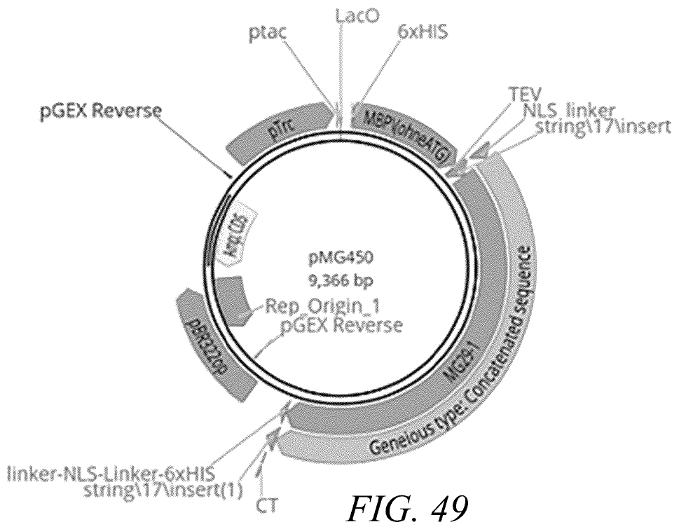

Expression of MG29-1 from the pMG450 vector depicted in FIG. 49 is tested in a screen varying the following conditions: host strain, expression media, inducer, induction time, and temperature.

After screening, E. coli is transformed with the appropriate expression plasmid, the culture is grown to a suitable density shake flasks, and the culture is induced using materials and methods according to the optimal expression conditions identified during the expression screen. The cell paste is harvested and expression is verified by SDS-PAGE. For these experiments, the cell culture volume is limited to 20 L. Up to 1 gram of protein is purified using the following method, formulated into storage buffer, and yield and concentration by A280 and purity by SDS-PAGE is assessed.

### Purification:

Total soluble protein extracted from E. coli cell paste is analyzed by SDS-PAGE for all conditions. Immobilized metal affinity chromatography (IMAC) pull-down followed by SDS-PAGE is performed on the top three expression conditions to estimate yield and purity and to identify the optimal expression condition. A scaled-up method is developed for lysis. Critical parameters are identified for purification by IMAC and subtractive IMAC (including tobacco etch virus protease (TEV) cleavage). Column fractions are tested using SDS-PAGE. Elution pools are tested using SDS-PAGE and photometric absorbance at 280 nm (A280). A method for buffer exchange and concentration by tangential flow filtration (TFF) is developed.

An additional chromatography stage is developed to achieve ≥90% purity, if purity is lower than 90%. One chromatography mode is tested (e.g., ceramic hydroxyapatite chromatography). Up to 8 unique conditions are tested (e.g., 2-6 resins each with 2-3 buffer systems). Column fractions are tested using SDS-PAGE. Elution pools are tested using SDS-PAGE and A280. One condition is selected, and a three-condition load study is performed. Column fractions and elution pools are analyzed as described above. A method for buffer exchange may be developed and concentration by TFF.

### Formulation Study

Using purified protein, a formulation study is conducted to determine the optimal storage conditions for the purified protein. Study may explore concentration, storage buffer, storage temperature, maximum freeze/thaw cycles, storage time, or other conditions.

### Example 29—Demonstration of the Ability of Nucleases Described Herein to Edit an Intronic Region in Cultured Mouse Liver Cells

Intronic regions of expressed genes are attractive genomic targets to integrate a coding sequence of a therapeutic protein of interest with the goal of expressing that protein to treat or cure a disease. Integration of a protein coding sequence may be accomplished by creating a double strand break within the intron using a sequence specific nuclease in the presence of an exogenously supplied donor template. The donor template may be integrated into the double strand break via one of two main cellular repair pathways called homology directed repair (HDR) and non-homologous end joining (NHEJ) resulting in targeted integration of the donor template. The NHEJ pathway is dominant in non-dividing cells while the HDR pathway is primarily active in dividing cells. The liver is a particularly attractive tissue for targeted integration of a protein coding sequence due to the availability of in vivo delivery systems and the ability of the liver to express and secrete proteins with high efficiency.

To evaluate the potential of MG29-1 to create double strand breaks at intronic regions the intron 1 of serum albumin was selected as the target locus. Single guide RNA (sgRNA) with a spacer length of 22 nt targeted to mouse albumin intron 1 were identified using the guide finding algorithm in the Geneious Prime nucleic acid analysis software (https://www.geneious.com/prime/). Using a PAM of KTTG (Sequence Number: A3870) located 5′ to the spacer, a total of 112 potential sgRNA were identified within mouse albumin intron 1. Guides that spanned the intron/exon boundaries were excluded. Using Geneious Prime the spacer sequences of these 112 guides were searched against the mouse genome and a specificity score was assigned by the software based on the alignment to additional sites in the genome. Spacer sequences with 4 or more contiguous bases of the same base were excluded due to concerns about specificity. A total of 12 spacers with the highest specificity scores were selected for testing. To create the sgRNA the backbone sequence of “TAATTTCTACTGTTGTAGAT” was added to the 3′ end of the spacer sequence. The sgRNA was chemically synthesized incorporating chemically modified bases identified to improve the performance of sgRNA for cpf1 guides (AltR1/AltR2 chemistry available from Integrated DNA Technologies). The spacer sequences of these guides are listed in Table 8 below.

TABLE 8
Activity of MG29-1 sgRNA targeting mouse albumin intron 1 in Hepa1-6 cells
nucleofected with MG29-1/sgRNA RNP or transfected with MG29-1 mRNA and sgRNA
using Messenger Max

| | | | | | | Activity | |
|---|---|---|---|---|---|---|---|
| | | | | | | (INDEL %) | |
| | | | | | | in | |
| | | | | | | Hepa1-6 | |
| | | | | | | cells | mRNA/s |
| | Spacer | SEQ | | Se- | | RNP | gRNA |
| sgRNA | (DNA sequence, | ID | | quence | Specificity | nucleo- | lipid |
| name | no PAM) | NO: | PAM | Number | score | fection | transfection |
| mAlb29-1-1 | GTATAGCATGGTCGAGCAG | 3993 | TTTA | A4012 | 98.5 | 86.5 | 43 |
| | GCA | | | | | | |
| mAlb29-1-2 | CCGATCGTTACAGGAAAAT | 3994 | GTTC | A4013 | 98.4 | 0 | 0 |
| | CTG | | | | | | |
| mAlb29-1-3 | AATTTATTACGGTCTCATA | 3995 | GTTG | A4014 | 98.2 | 0 | 0 |
| | GGG | | | | | | |
| mAlb29-1-4 | TTACGGTCTCATAGGGCCT | 3996 | TTTA | A4015 | 97.6 | 43.5 | 44 |
| | GCC | | | | | | |
| mAlb29-1-5 | CCTGTAACGATCGGGAACT | 3997 | TTTT | A4016 | 97.2 | 3 | 0 |
| | GGC | | | | | | |
| mAlb29-1-7 | AGTATAGCATGGTCGAGCA | 3998 | TTTT | A4017 | 96.8 | 11 | 15 |
| | GGC | | | | | | |
| mAlb29-1-8 | CTGTAACGATCGGGAACTG | 3999 | TTTC | A4018 | 95.9 | 77 | 45 |
| | GCA | | | | | | |
| mAlb29-1-9 | GATACAGTTGAATTTATTA | 4000 | GTTG | A4019 | 95.3 | 0 | 0 |
| | CGG | | | | | | |
| mAlb29-1- | TAGTATAGCATGGTCGAGC | 4001 | TTTT | A4020 | 95.2 | 18 | 35 |
| 10 | AGG | | | | | | |
| mAlb29-1- | CATCTGAGAACCCTTAGGT | 4002 | TTTG | A4021 | 95.0 | 7 | 2 |
| 11 | GGT | | | | | | |
| mAlb29-1- | AGTGTAGCAGAGAGGAACC | 4003 | TTTG | A4022 | 93.8 | NT | 47 |
| 12 | ATT | | | | | | |
| mAlb29-1- | CTAGTAATGGAAGCCTGGT | 4004 | TTTT | A4023 | 92.4 | 8 | 24 |
| 13 | ATT | | | | | | |
| mAlb29-1- | GGTATCTTTGATGACAATA | 4005 | TTTT | A4024 | 91.8 | 0 | 13 |
| 14 | ATG | | | | | | |
| mAlb29-1- | TCTAGTAATGGAAGCCTGG | 4006 | TTTT | A4025 | 91.8 | 0 | 0 |
| 15 | TAT | | | | | | |
| mAlb29-1- | TAGTAATGGAAGCCTGGTA | 4007 | TTTC | A4026 | 89.8 | 90.5 | 51 |
| 16 | TTT | | | | | | |
| mAlb29-1- | GTATCTTTGATGACAATAA | 4008 | TTTG | A4027 | 87.8 | 10 | NT |
| 17 | TGG | | | | | | |
| mAlb29-1- | AAGATTGATGAAGACAACT | 4009 | TTTA | A4028 | 87.4 | 76 | NT |
| 18 | AAC | | | | | | |
| mAlb29-1- | CTCTCTGCTACACTCAAAG | 4010 | GTTC | A4029 | 85.7 | 0 | 0 |
| 19 | TTA | | | | | | |
| mAlb29-1- | AAACCCGTTAAGTGTTTAT | 4011 | TTTA | A4030 | 87.3 | 0 | 4 |
| 20 | ATC | | | | | | |

Hepa1-6 cells, a transformed mouse liver cell line, were cultured under standard conditions (DMEM media with 10% FBS in 5% CO2 incubator) and nucleofected with ribonuclear proteins formed by mixing the sgRNA and purified MG29-1 protein in PBS buffer. Hepa1-6 cells (1×10⁵) in suspension in complete SF nucleofection reagent (Lonza) were nucleofected using a 4D nucleofection device (Lonza) with RNP formed by mixing 50 pmol of MG29-1 protein and 100 pmol of sgRNA. After nucleofection the cells were plated in 24 well plates in DMEM plus 10% FBS and incubated in a 5% CO2 incubator for 48 to 72 h. Genomic DNA was then extracted from the cells using a column-based purification kit (Purelink genomic DNA mini kit, ThermoFisher Scientific) and quantified by absorbance at 260 nm. The albumin intron 1 region was PCR amplified from 50 ng of the genomic DNA in a reaction containing 0.5 micro molar each of the primers mA1b90F (CTCCTCTTCGTCTCCGGC) (SEQ ID NO: 4031) and mA1b1073R (CTGCCACATTGCTCAGCAC) (SEQ ID NO: 4032) and 1× Pfusion Flash PCR Master Mix.

The resulting 984 bp PCR product which spans the entire intron 1 of mouse albumin was purified using a column-based purification kit (DNA Clean and Concentrator, Zymo Research) and sequenced using primers located within 150 to 350 bp of the predicted target site for each sgRNA. A PCR product generated using primers mA1b90F (SEQ ID NO: 4031) and mA1b1073R (SEQ ID NO: 4032) from un-transfected Hepa1-6 cells was sequenced in parallel as a control. The Sanger sequencing chromatograms were analyzed using Inference of CRISPR Edits (ICE) that determines the frequency of INDELS as well as the INDEL profile (Hsiau et. al, Inference of CRISPR Edits from Sanger Trace Data. BioArxiv. 2018 https://www.biorxiv.org/content/early/2018/01/20/251082).

When a nuclease creates a double strand break (DSB) in DNA inside a living cell the DSB is repaired by the cellular DNA repair machinery. In actively dividing cells such as transformed mammalian cells in culture, and in the absence of a repair template, this repair occurs by the NHEJ pathway. The NHEJ pathway is an error prone process that introduces insertions or deletions of bases at the site of the double strand break (Lieber, M. R, Annu Rev Biochem. 2010; 79: 181-211). These insertions and deletions are therefore a hallmark of a double strand break that occurred and was subsequently repaired, is widely used as a readout of the editing or cutting efficiency of the nuclease. The profile of insertions and deletions depends on the characteristics of the nuclease that created the double strand break but also upon the sequence context at the cleavage site. Based on in vitro assays, the MG29-1 nuclease creates a staggered cut located 3′ of the PAM. Staggered cuts will often lead to larger deletions due to the trimming of the single stranded ends before end-joining. Table 8 lists the total INDEL frequency generated by each of the 19 sgRNA targeting mouse albumin intron 1 that were tested in Hepa1-6 cells. Eleven of the 18 sgRNA resulted in detectable INDELS at the target site with 5 sgRNA resulting in INDEL frequencies greater than 50% and 4 sgRNA resulted in indel frequencies greater than 75%. These data demonstrate that the MG29-1 nuclease can edit the genome of cultured mouse liver cells at the predicted target site for the sgRNA with efficiencies greater than 75%.

The editing efficiencies of the same set of sgRNA were evaluated by co-transfection of the sgRNA and a mRNA encoding the MG29-1 nuclease using a commercial lipid-based transfection reagent (Lipofectamine MessengerMAX, Invitrogen). The mRNA encoding MG29-1 was generated by in vitro transcription using T7 polymerase from a plasmid in which the coding sequence of MG29-1 was cloned. The MG29-1 coding sequence was codon optimized using human codon usage tables and flanked by nuclear localization signals derived from SV40 at the N-terminus and from Nucleoplasmin at the C-terminus. In addition, a UTR was included at the 3′ end of the coding sequence to improve translation. A 3′ UTR followed by an approximately 90 to 110 nucleotide poly A tract was included at the 3′ end of the coding sequence to improve mRNA stability in vivo (see e.g. SEQ ID NO: 4426 for wild-type MG29-1 and SEQ ID NO: 3327 for the S168R variant). The in vitro transcription reaction included the Clean Cap® capping reagent (Trilink BioTechnologies) and the resulting RNA was purified using the MEGAClear™ Transcription Clean-Up kit (Invitrogen) and purity was evaluated using the TapeStation (Agilent) and found to be composed of >90% full length RNA. As seen in Table 1, the editing efficiencies after mRNA/sgRNA lipid transfection of Hepa1-6 cells were similar but not identical to those seen with nucleofection of RNP but confirm that the MG29-1 nuclease is active in cultured liver cells when delivered in the form of an mRNA.

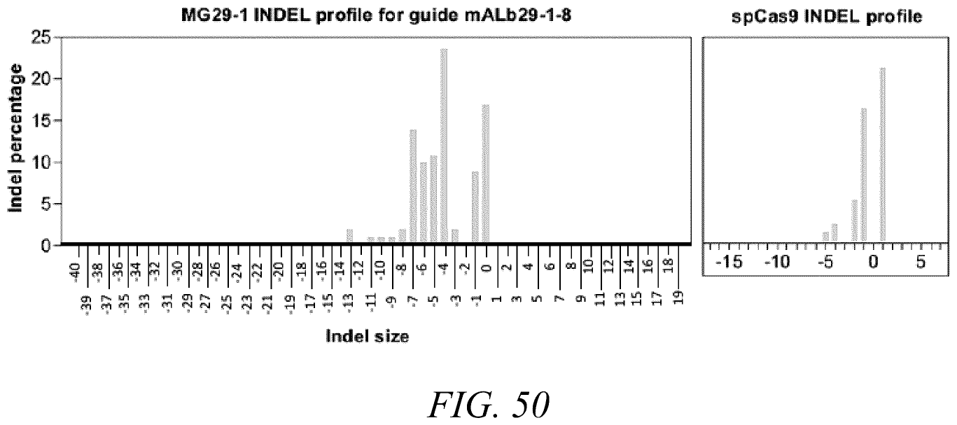

FIG. 50 is a representative example of the indel profile of MG29-1 as determined by ICE analysis using mALb29-1-8 as the guide (SEQ ID NO: 3999) and demonstrates that deletion of 4 bases was the most frequent event (25% of total sequences) and deletions of 1, 5, 6, or 7 bases each accounting for about 10 to 15% of the sequences. Longer deletions of up to 13 bases were also detected, but insertions were undetectable. By contrast, spCas9 with a guide targeting mouse albumin intron 1 generated primarily 1 base insertions or deletions.

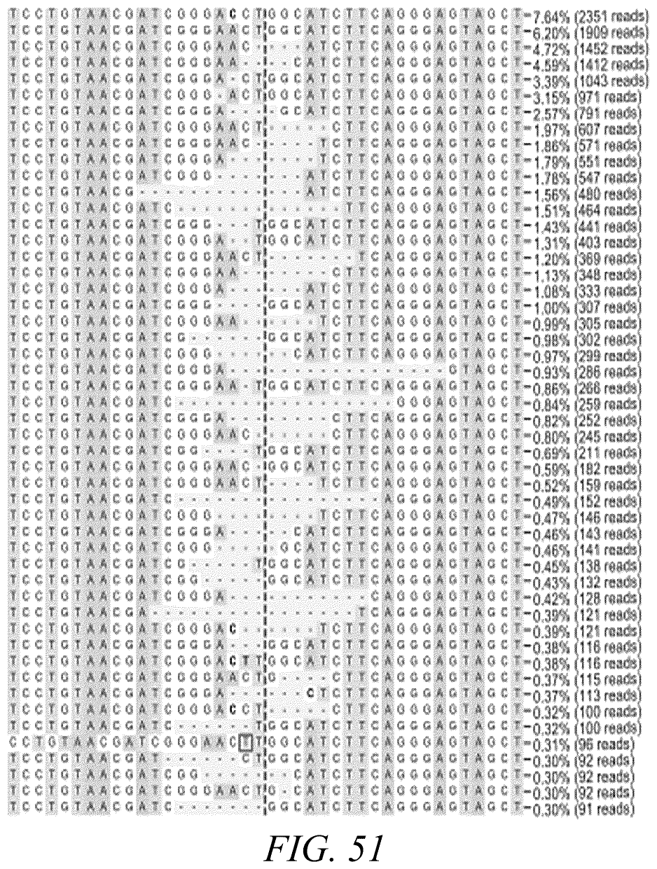

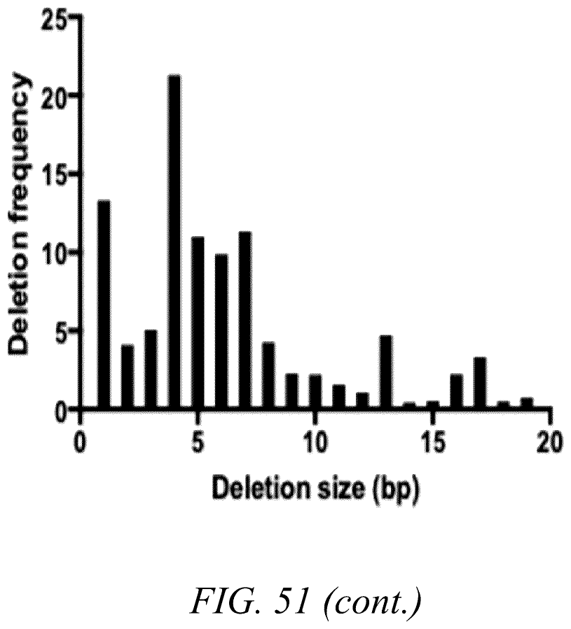

FIG. 51 is a representative example of the indel profile of MG29-1 and sgRNA mA1b29-1-8 as determined by next generation sequencing (NGS) of the PCR product of the mouse albumin intron 1 region. In total approximately 15,000 sequence reads were obtained. By NGS deletion of 4 bases was the most frequent indel (about 20% of total) with deletions of 1, 5, 6 and 7 bases each accounting for about 10% of the indels. Larger deletions of up to 19 bp were also detected. The profile observed by NGS analysis matches closely that measured by ICE. These results demonstrate that MG29-1 generates large deletions at the target site consistent with the staggered cleavage observed in vitro.

### Example 8—Demonstration of the Ability of a Nuclease Described Herein to Target an Intronic Region in Cultured Human Liver Cells (HepG2)

To evaluate the potential of MG29-1 to create double strand breaks at intronic regions in human cells, the intron 1 of human serum albumin was selected as the target locus. Single guide RNA (sgRNA) with a spacer length of 22 nt targeted to human albumin intron 1 were identified using the guide finding algorithm in the Geneious Prime nucleic acid analysis software (https://www.geneious.com/prime/). Using a PAM of KTTG (Sequence Number: A3870) located 5′ to the spacer, a total of 90 potential sgRNA were identified within human albumin intron 1. Guides that spanned the intron/exon boundaries were excluded. Using Geneious Prime the spacer sequences of these guides were searched against the mouse genome and a specificity score was assigned by the software based on the alignment to additional sites in the genome. Spacer sequences with 4 or more contiguous bases of the same base were excluded due to concerns about specificity. A total of 23 spacers with the highest specificity scores were selected for testing. To create the sgRNA the backbone sequence of “TAATTTCTACTGTTGTAGAT” was added to the 3′ end of the spacer sequence. The sgRNA was chemically synthesized incorporating chemically modified bases identified to improve the performance of sgRNA for cpf1 guides (AltR1/AltR2 chemistry available from Integrated DNA Technologies). The spacer sequences of these guides are listed in Table 9.

TABLE 9
Spacer sequences of MG29-1 sgRNA targeting human albumin intron 1 and
activity in HepG2 cells nucleofected with MG29-1/sgRNA RNP

| | | | | | | Activity |
|---|---|---|---|---|---|---|
| | | | | | | (INDEL |
| | | SEQ | | | | %) in |
| sgRNA | | ID | | Sequence | Specificity | HepG2 |
| name | Spacer (DNA sequence, no PAM) | NO: | PAM | Number: | score | cells |
| hAlb_g63 | GTAAACTCTGCATCTTTAAAGA | 4033 | TTTA | A4056 | 91.25% | 0 |
| hAlb_g59 | TTTCAAAATATTGGGCTCTGAT | 4034 | TTTG | A4057 | 90.64% | 0 |
| hAlb_g58 | AGTAAACTCTGCATCTTTAAAG | 4035 | TTTT | A4058 | 90.46% | 0 |
| hAlb_g56 | AAGATGCAGAGTTTACTAAAAC | 4036 | TTTA | A4059 | 90.23% | 0 |
| hAlb_g72 | AAAATATTGGGCTCTGATTCCT | 4037 | TTTC | A4060 | 93.26% | 0 |
| hAlb_g70 | AAATAAAGCATAGTGCAATGGA | 4038 | TTTT | A4061 | 92.31% | 63 |
| hAlb_g74 | AATAAAGCATAGTGCAATGGAT | 4039 | TTTA | A4062 | 95.41% | 93 |
| hAlb_g83 | TGAGATCAACAGCACAGGTTTT | 4040 | TTTA | A4063 | 98.39% | 93 |
| hAlb_g85 | TGTAGGAATCAGAGCCCAATAT | 4041 | TTTC | A4064 | 99.20% | 55 |
| hAlb_g89 | CTGTAGGAATCAGAGCCCAATA | 4042 | TTTT | A4065 | 100.00% | 43 |
| hAlb_g88 | TCTGTAGGAATCAGAGCCCAAT | 4043 | TTTT | A4066 | 100.00% | 45 |
| hAlb_g77 | GTGACTGTAATTTTCTTTTGCG | 4044 | TTTA | A4067 | 96.77% | 0 |
| hAlb_g69 | CTTTTGCGCACTAAGGAAAGTG | 4045 | TTTT | A4068 | 92.18% | 3 |
| hAlb_g66 | TGAAGTCTTACAAGGTTATCTT | 4046 | TTTG | A4069 | 91.80% | 19 |
| hAlb_g75 | AGTGTCTATCAACAGCAACCAA | 4047 | TTTT | A4070 | 95.96% | 13 |
| hAlb_g79 | CTTAGTGCGCAAAAGAAAATTA | 4048 | TTTC | A4071 | 97.45% | 60 |
| hAlb_g82 | TAGCCTTATATTCAAACTTAGA | 4049 | TTTA | A4072 | 98.32% | 0 |
| hAlb_g80 | GGATAGTTATGAATTCAATCTT | 4050 | TTTG | A4073 | 97.46% | 23 |
| hAlb_g84 | CACTTTCCTTAGTGCGCAAAAG | 4051 | TTTG | A4074 | 98.85% | 96 |
| hAlb_g81 | GTATTTGTGAAGTCTTACAAGG | 4052 | TTTT | A4075 | 98.07% | 17 |
| hAlb_g90 | GTGTCTATCAACAGCAACCAAG | 4053 | TTTA | A4076 | 100.00% | 91 |
| hAlb_g87 | CGCACTAAGGAAAGTGCAAAGT | 4054 | TTTG | A4077 | 100.00% | 97 |
| hAlb_g86 | GCGCACTAAGGAAAGTGCAAAG | 4055 | TTTT | A4078 | 100.00% | 42 |

HepG2 cells, a transformed human liver cell line, were cultured under standard conditions (MEM media with 10% FBS in 5% CO2 incubator) and nucleofected with ribonuclear proteins formed by mixing the sgRNA and purified MG29-1 protein in PBS buffer. A total of 1 e5 HepG2 cells in suspension in complete SF nucleofection reagent (Lonza) were nucleofected using a 4D nucleofection device (Lonza) with RNP formed by mixing 80 pmol of MG29-1 protein and 160 pmol of sgRNA. After nucleofection the cells were plated in 24 well plates in DMEM plus 10% FBS and incubated in a 5% CO₂ incubator for 48 to 72 h. Genomic DNA was then extracted from the cells using a column-based purification kit (Purelink genomic DNA mini kit, ThermoFisher Scientific) and quantified by absorbance at 260 nm. The albumin intron 1 region was PCR amplified from 50 ng of the genomic DNA in a reaction containing 0.5 micro molar each of the primers hA1b 11F (TCTTCTGTCAACCCCACACGCC) (SEQ ID NO: 4079) and hA1b834R (CTGTCTGGGCAAGGGAAGA) (SEQ ID NO: 4080) and 1× Pfusion Flash PCR Master Mix. The resulting 826 bp PCR product which spans the entire intron 1 of mouse albumin was purified using a column-based purification kit (DNA Clean and Concentrator, Zymo Research) and sequenced using primers located within 150 to 350 bp of the predicted target site for the sgRNA.

The PCR product generated using primers hA1b 11F (TCTTCTGTCAACCCCACACGCC) (SEQ ID NO: 4079) and hA1b834R (CTTGTCTGGGCAAGGGAAGA) (SEQ ID NO: 4080) from un-transfected HepG2 cells was sequenced in parallel as a control. The Sanger sequencing chromatograms were analyzed using Inference of CRISPR Edits (ICE) that determines the frequency of INDELS as well as the INDEL profile. When a nuclease creates a double strand break (DSB) in DNA inside a living cell the DSB is repaired by the cellular DNA repair machinery. In actively dividing cells such as transformed mammalian cells in culture, and in the absence of a repair template, this repair occurs by the NHEJ pathway. The NHEJ pathway is an error prone process that introduces insertions or deletions of bases at the site of the double strand break (Lieber, M. R, Annu Rev Biochem. 2010; 79: 181-211).

These insertions and deletions are therefore a hallmark of a double strand break that occurred and was subsequently repaired, and is widely used as a readout of the editing or cutting efficiency of the nuclease. The profile of insertions and deletions depends on the characteristics of the nuclease that created the double strand break but also upon the sequence context at the cleavage site. Based on in vitro assays, the MG29-1 nuclease cleaves the target strand at 22 nucleotides from the PAM (less frequently at 21 nucleotides from the PAM) and cleaves the non-target strand at 18 nucleotides from the PAM which therefore creates 4 nucleotide staggered end located 3′ of the PAM. Staggered cuts will often lead to larger deletions due to the trimming of the single stranded ends before end-joining.

Table 9 lists the total indel frequency generated by each of the 23 sgRNA targeting human albumin intron 1 that were tested in HepG2 cells. Sixteen of the 23 sgRNA resulted in detectable indel at the target site with 8 sgRNA resulting in INDELS greater than 50% and 5 sgRNA resulted in indel frequencies than 90%. These data demonstrate that the MG29-1 nuclease can edit the genome of a cultured human liver cell line at the predicted target site for the sgRNA with efficiencies greater than 90%.

### Example 9—Demonstration of the Ability of Nucleases Described Herein to Edit Exonic Regions in Cultured Mouse Liver Cells

Sequence specific nucleases can be used to disrupt the coding sequences of genes and thereby create a functional knockout of a protein of interest. This can be of therapeutic use when the knockdown of a specific protein has a beneficial effect in a particular disease. One way to disrupt the coding sequence of a gene is to make a double strand break within the exonic regions of the gene using a sequence specific nuclease. These double strand breaks will be repaired via error prone repair pathways to generate insertions or deletions which can result in either frameshift mutations or changes to the amino acid sequence which disrupt the function of the protein.

To evaluate the potential of MG29-1 to create double strand breaks at exonic regions of a gene expressed in liver cells the gene encoding glycolate oxidase (hao-1) was selected as the target locus. Single guide RNA (sgRNA) with a spacer length of 22 nt targeted to exons 1 to 4 of mouse hao-1 were identified using the guide finding algorithm in the Geneious Prime nucleic acid analysis software (https://www.geneious.com/prime/). The first 4 exons of the hao-1 gene comprise approximately the N-terminal 50% of the hao-1 coding sequence. The first 4 exons were chosen because INDELS created towards the N-terminus of the coding sequence of a gene are more likely to create a frameshift or missense mutation that disrupts the activity of the protein. Using a PAM of KTTG (Sequence Number: A3870) located 5′ to the spacer, a total of 45 potential sgRNAs were identified within mouse hao-1 exons 1 through 4. Guides that spanned the intron/exon boundaries were included because such guides may create INDELS that interfere with splicing. Using Geneious Prime, the spacer sequences of these 45 guides were searched against the mouse genome and a specificity score was assigned by the software based on the alignment to additional sites in the mouse genome. Spacer sequences with 4 or more contiguous bases of the same base were excluded due to concerns about specificity. A total of 45 spacers with the highest specificity scores were selected for testing.

To create the sgRNA the backbone sequence of “TAATTTCTACTGTTGTAGAT” was added to the 3′ end of the spacer sequence. The sgRNA was chemically synthesized incorporating chemically modified bases identified to improve the performance of sgRNA for cpf1 guides (A1tR1/A1tR2 chemistry available from Integrated DNA Technologies). The spacer sequences of these guides are listed in Table 3.

TABLE 3
Spacer sequences of MG29-1 sgRNA targeting mouse hao-1 exons 1 to 4 and
activity in Hepa1-6 cells nucleofected with MG29-1/sgRNA RNP

| | | | | | | Activity |
|---|---|---|---|---|---|---|
| | | | | | | (INDEL |
| | Spacer | SEQ | | | | %) in |
| sgRNA | (DNA sequence, no | ID | | Sequence | Specificity | Hepa1-6 |
| name | PAM) | NO: | PAM | Number: | score | cells |
| mH29-1 | CCCCAGACCTGTAATAGTCATA | 4081 | TTTG | A4126 | 100.00% | 92.2 |
| mH29-2 | AGGACAGAGAGTCAGCATGCCA | 4082 | TTTT | A4127 | 100.00% | 0 |
| mH29-3 | GGAGACAACAGTGGACTTGCTG | 4083 | TTTT | A4128 | 100.00% | 0 |
| mH29-4 | CCCTACCCTGCCACAATGTTGC | 4084 | GTTG | A4129 | 100.00% | 0 |
| mH29-5 | CTTACCTAGAAAATGCTTGGAT | 4085 | GTTT | A4130 | 100.00% | 0 |
| mH29-6 | ACAGATCGATATCAGCAACGTT | 4086 | GTTG | A4131 | 100.00% | 0 |
| mH29-7 | CGAAGCATCCGTGGATAGAGCT | 4087 | GTTG | A4132 | 100.00% | 0 |
| mH29-8 | TTGGGCTACCTCCTCAATAGAA | 4088 | GTTC | A4133 | 100.00% | 0 |
| mH29-9 | AAGCTGCCACCACAACTCAGGT | 4089 | GTTC | A4134 | 100.00% | 0 |
| mH29-10 | TGGTGGCAGCTTGAACCTGTTC | 4090 | GTTG | A4135 | 100.00% | 0 |
| mH29-11 | CGCACGTCATCAATGCGGTTGC | 4091 | GTTC | A4136 | 100.00% | 0 |
| mH29-12 | CCCAGGTAAGGGGTGTCCACAG | 4092 | GTTG | A4137 | 100.00% | 0 |
| mH29-13 | CATCCAGCGAAGTGCCTCTGGG | 4093 | GTTG | A4138 | 100.00% | 0 |
| mH29-14 | AAATTCCAGATGGAAGCTCTAT | 4094 | TTTT | A4139 | 99.04% | 0 |
| mH29-15 | TGACTGTGGACACCCCTTACCT | 4095 | TTTG | A4140 | 99.04% | 97.5 |
| mH29-16 | ATTACAGCCTGTCAGACCATGG | 4096 | TTTC | A4141 | 99.30% | 91 |
| mH29-17 | GAGACAACAGTGGACTTGCTGA | 4097 | TTTG | A4142 | 98.37% | 27.5 |
| mH29-18 | CAACAATAGGCAGTGATGTCAA | 4098 | TTTA | A4143 | 99.22% | 85 |
| mH29-19 | CCTCGACTGGTCTGCATCAGTG | 4099 | GTTG | A4144 | 97.69% | 0 |
| mH29-20 | ATAATCACTGATGCAGACCAGT | 4100 | GTTC | A4145 | 98.07% | 0 |
| mH29-21 | TCAGCTAACGTCTCCTGATCAT | 4101 | GTTA | A4146 | 99.33% | 0 |
| mH29-22 | CTGATATCGATCTGTCAACTTC | 4102 | GTTG | A4147 | 99.22% | 0 |
| mH29-23 | TAAAGGGCATTTTGAGAGGTTT | 4103 | GTTG | A4148 | 99.22% | 0 |
| mH29-24 | AATAGCAAAGTTTCTTACCTAG | 4104 | TTTA | A4149 | 95.75% | 0 |
| mH29-25 | GGACAGAGAGTCAGCATGCCAA | 4105 | TTTA | A4150 | 95.73% | 47 |
| mH29-26 | TCCATTTCATTACAGCCTGTCA | 4106 | TTTC | A4151 | 94.08% | 79 |
| mH29-27 | AGTCTGTGAGATCATACTGACC | 4107 | TTTG | A4152 | 96.85% | 65 |
| mH29-28 | TAGATGTACAGTTGCATCCAGC | 4108 | TTTG | A4153 | 98.20% | 29 |
| mH29-29 | CCTTAGGAGAAAATGCCAAATC | 4109 | TTTC | A4154 | 96.18% | 94.5 |
| mH29-30 | CTCCTAAGGGAAATTTTGGAGA | 4110 | TTTT | A4155 | 98.06% | 0 |
| mH29-31 | GCTGATAACATCCAAGCATTTT | 4111 | GTTA | A4156 | 93.30% | 0 |
| mH29-32 | AAATAGCAAAGTTTCTTACCTA | 4112 | GTTT | A4157 | 97.31% | 0 |
| mH29-33 | TAGGACAGAGAGTCAGCATGCC | 4113 | GTTT | A4158 | 95.84% | 0 |
| mH29-34 | GGGCTACTGCCATGCAGTGCAT | 4114 | GTTG | A4159 | 97.53% | 0 |
| mH29-35 | TCTCCAAAATTTCCCTTAGGAG | 4115 | GTTG | A4160 | 96.94% | 0 |
| mH29-36 | AATTCCAGATGGAAGCTCTATC | 4116 | TTTA | A4161 | 96.74% | 0 |
| mH29-37 | TCCTAAGGGAAATTTTGGAGAC | 4117 | TTTC | A4162 | 97.59% | 53.5 |
| mH29-38 | TTACCTAGAAAATGCTTGGATG | 4118 | TTTC | A4163 | 94.99% | 21.8 |
| mH29-39 | CAAGGCCATATTTGTGACTGTG | 4119 | GTTA | A4164 | 93.57% | 0 |
| mH29-40 | CTCCATTTCATTACAGCCTGTC | 4120 | TTTT | A4165 | 93.71% | 0 |
| mH29-41 | GCATTTTCTCCTAAGGGAAATT | 4121 | TTTG | A4166 | 92.30% | 59 |
| mH29-42 | TTACCTCGCACAGTGGCCAGCT | 4122 | TTTC | A4167 | 77.16% | 32 |
| mH29-43 | TCTCTCTTTTCTTACCTCGCAC | 4123 | TTTG | A4168 | 87.70% | 0 |
| mH29-44 | CTTACCTCGCACAGTGGCCAGC | 4124 | TTTT | A4169 | 95.19% | 0 |
| mH29-45 | AAACCAATGATTTGGCATTTTC | 4125 | TTTG | A4170 | 91.09% | 0 |

Hepa1-6 cells, a transformed mouse liver cell line, were cultured under standard conditions (DMEM media with 10% FBS in 5% CO₂ incubator) and nucleofected with ribonuclear proteins formed by mixing the sgRNA and purified MG29-1 protein in PBS buffer. A total of 1 e⁵ Hepa1-6 cells in suspension in complete SF nucleofection reagent (Lonza) were nucleofected using a 4D nucleofection device (Lonza) with RNP formed by mixing 50 pmol of MG29-1 protein and 100 pmol of sgRNA. After nucleofection the cells were plated in 24 well plates in DMEM plus 10% FBS and incubated in a 5% CO2 incubator for 48 to 72 h. Genomic DNA was then extracted from the cells using a column-based purification kit (Purelink genomic DNA mini kit, ThermoFisher Scientific) and quantified by absorbance at 260 nm. Exons 1 through 4 of the mouse hao-1 gene 1 were PCR amplified from 40 ng of the genomic DNA in a reaction containing 0.5 micro molar pairs of the primers specific for each exon. The PCR primers used for exon 1 were PCR_mHE1_F_+233 (GTGACCAACCCTACCCGTTT) (SEQ ID NO: 4171), PCR_mHE1_R_-553 (GCAAGCACCTACTGTCTCGT) (SEQ ID NO: 4172). The PCR primers used for exon 2 were HAO1_E2_F5721 (CAACGAAGGTTCCCTCCAGG) (SEQ ID NO: 4173), HAO1_E2_R6271 (GGAAGGGTGTTCGAGAAGGA) (SEQ ID NO: 4174). The PCR primers used for exon 3 were HAO1_E3_F23198 (TGCCCTAGACAAGCTGACAC) (SEQ ID NO: 4175), HAO1_E3_R23879 (CAGATTCTGGAAGTGGCCCA) (SEQ ID NO: 4176). The PCR primers used for exon 4 were HAO1_E4_F31087 (CCTGTAGGTGGCTGAGTACG) (SEQ ID NO: 4177), HAO1_E4_R31650 (AGGTTTGGTTCCCCTCACCT) (SEQ ID NO: 4178).

In addition to primers and genomic DNA the PCR reactions contained 1× Pfusion Flash PCR Master Mix (Thermo Fisher). The resulting PCR products comprised single bands when analyzed on agarose gels demonstrating that the PCR reaction was specific, and were purified using a column-based purification kit (DNA Clean and Concentrator, Zymo Research). For sequencing, primers complementary to sequences at least 100 nt from each cut site were used. The primer to sequence Exon 1 was Seq_mHE1_F_+139 (GTCTAGGCATACAATGTTTGCTCA) (SEQ ID NO: 4179). The primer to sequence Exon 2 was 5938F Seq_HAO1_E2 (CTATGCAAGGAAAAGATTTGGCC) (SEQ ID NO: 4180). The primers to sequence Exon 3 were HAO1_E3_F23476 (TCTCCCCCTGAATGAAACACT) (SEQ ID NO: 4181) and the reverse PCR primer, HAO1_E3_R23879 (CAGATTCTGGAAGTGGCCCA) (SEQ ID NO: 4182). The primer to sequence Exon 4 was the reverse PCR primer, HAO1_E4_R31650 (AGGTTTGGTTCCCCTCACCT) (SEQ ID NO: 4183).

Sequencing of the PCR products showed that they contained the expected sequences of the hao-1 exons. PCR products derived from Hepa-16 cells nucleofected with different RNP or untreated controls were sequenced using primers located within 100 to 350 bp of the predicted target site for each sgRNA. The Sanger sequencing chromatograms were analyzed using Inference of CRISPR Edits (ICE) that determines the frequency of INDELS as well as the INDEL profile (Hsiau et. al, Inference of CRISPR Edits from Sanger Trace Data. BioArxiv. 2018 https://www.biorxiv.org/content/early/2018/01/20/251082). When a nuclease creates a double strand break (DSB) in DNA inside a living cell the DSB is repaired by the cellular DNA repair machinery. In actively dividing cells such as transformed mammalian cells in culture, and in the absence of a repair template, this repair occurs by the NHEJ pathway. The NHEJ pathway is an error prone process that introduces insertions or deletions of bases at the site of the double strand break (Lieber, M. R, Annu Rev Biochem. 2010; 79: 181-211). These insertions and deletions are therefore a hallmark of a double strand break that occurred and was subsequently repaired, and is widely used in the art as a readout of the editing or cutting efficiency of the nuclease. As presented in Table 3, 14 guides demonstrated detectable editing at their predicted target sites. Four guides exhibited editing activity greater than 90%. All 14 of the active guides had PAM sequences of TTTN demonstrating that this PAM is more efficient in vivo. However not all guides utilizing a TTTN PAM were active. These data demonstrate that the MG29-1 nuclease can generate RNA guided, sequence specific, double strand breaks in exonic regions in cultured liver cells with high efficiency.

### Example 10—Design of Further sgRNAs for Disruption of Hao-1 Gene

Further sgRNAs were designed to target exonic parts of the hao-1 gene. These are designed to target the first 4 exons because these comprise approximately 50% of the coding sequence and indels created towards the N-terminus of the coding sequence of a gene are more likely to create a frameshift or missense mutation that disrupts the activity of the protein. Using the more restrictive PAM of KTTG (Sequence Number: A3870) which was shown in Example 9 to be more active in mammalian cells, a total of 42 potential sgRNA were identified within human hao-1 exons 1 through 4 (Table 4).

TABLE 4
Spacer sequences for MG29-1 identified in
exons 1 to 4 of the human hao-1 gene

| sgRNA | Spacer (DNA sequence, | SEQ ID | | Sequence | Specificity |
|---|---|---|---|---|---|
| name | no PAM) | NO: | PAM | Number: | score |
| hH29-1 | GCATGTTGTTCATAATCATTGA | 4184 | TTTA | A4226 | 96.25% |
| hH29-2 | GAAGTACTGATTTAGCATGTTG | 4185 | TTTG | A4227 | 98.37% |
| hH29-3 | TATCAATGATTATGAACAACAT | 4186 | TTTG | A4228 | 87.44% |
| hH29-4 | CCCCAGACCTGTAATAGTCATA | 4187 | TTTG | A4229 | 99.04% |
| hH29-5 | TTCATCATTTGCCCCAGACCTG | 4188 | TTTC | A4230 | 95.59% |
| hH29-6 | TTACCTGGAAAATGCTGCAATA | 4189 | TTTC | A4231 | 80.36% |
| hH29-7 | CTTACCTGGAAAATGCTGCAAT | 4190 | TTTT | A4232 | 79.67% |
| hH29-8 | GCTGATAATATTGCAGCATTTT | 4191 | TTTG | A4233 | 92.20% |
| hH29-9 | AAAAATAAATTTTCTTACCTGG | 4192 | TTTA | A4234 | 58.56% |
| hH29-10 | AAAAAATAAATTTTCTTACCTG | 4193 | TTTT | A4235 | 44.93% |
| hH29-11 | ATTTTATTTTTTAATTCTAGAT | 4194 | TTTT | A4236 | 10.22% |
| hH29-12 | TTTTATTTTTTAATTCTAGATG | 4195 | TTTA | A4237 | 10.64% |
| hH29-13 | ATTTTTTAATTCTAGATGGAAG | 4196 | TTTT | A4238 | 70.62% |
| hH29-14 | TTTTTTAATTCTAGATGGAAGC | 4197 | TTTA | A4239 | 44.69% |
| hH29-15 | TTAATTCTAGATGGAAGCTGTA | 4198 | TTTT | A4240 | 99.13% |
| hH29-16 | TAATTCTAGATGGAAGCTGTAT | 4199 | TTTT | A4241 | 97.06% |
| hH29-17 | AATTCTAGATGGAAGCTGTATC | 4200 | TTTT | A4242 | 96.74% |
| hH29-18 | ATTCTAGATGGAAGCTGTATCC | 4201 | TTTA | A4243 | 98.94% |
| hH29-19 | AGCAACATTCCGGAGCATCCTT | 4202 | TTTC | A4244 | 97.81% |
| hH29-20 | AGGACAGAGGGTCAGCATGCCA | 4203 | TTTT | A4245 | 97.75% |
| hH29-21 | GGACAGAGGGTCAGCATGCCAA | 4204 | TTTA | A4246 | 100.00% |
| hH29-22 | TTTCTCAGCCTGTCAGTCCCTG | 4205 | TTTC | A4247 | 88.19% |
| hH29-23 | TCAGCCTGTCAGTCCCTGGGAA | 4206 | TTTC | A4248 | 100.00% |
| hH29-24 | TGACAGTGGACACACCTTACCT | 4207 | TTTG | A4249 | 100.00% |
| hH29-25 | AATCTGTTACGCACATCATCCA | 4208 | TTTG | A4250 | 100.00% |
| hH29-26 | ATGCATTTCTTATTTTAGGATG | 4209 | TTTT | A4251 | 80.79% |
| hH29-27 | TGCATTTCTTATTTTAGGATGA | 4210 | TTTA | A4252 | 76.81% |
| hH29-28 | TTATTTTAGGATGAAAAATTTT | 4211 | TTTC | A4253 | 52.38% |
| hH29-29 | AGGATGAAAAATTTTGAAACCA | 4212 | TTTT | A4254 | 90.56% |
| hH29-30 | GGATGAAAAATTTTGAAACCAG | 4213 | TTTA | A4255 | 89.17% |
| hH29-31 | CTCAGGAGAAAATGATAAAGTA | 4214 | TTTC | A4256 | 90.51% |
| hH29-32 | CCTCAGGAGAAAATGATAAAGT | 4215 | TTTT | A4257 | 88.16% |
| hH29-33 | GAAACCAGTACTTTATCATTTT | 4216 | TTTT | A4258 | 86.74% |
| hH29-34 | AAACCAGTACTTTATCATTTTC | 4217 | TTTG | A4259 | 91.02% |
| hH29-35 | TCATTTTCTCCTGAGGAAAATT | 4218 | TTTA | A4260 | 83.29% |
| hH29-36 | CTCCTGAGGAAAATTTTGGAGA | 4219 | TTTT | A4261 | 91.88% |
| hH29-37 | TCCTGAGGAAAATTTTGGAGAC | 4220 | TTTC | A4262 | 96.24% |
| hH29-38 | GCCACATATGCAGCAAGTCCAC | 4221 | TTTA | A4263 | 100.00% |
| hH29-39 | GGAGACGACAGTGGACTTGCTG | 4222 | TTTT | A4264 | 90.43% |
| hH29-40 | GAGACGACAGTGGACTTGCTGC | 4223 | TTTG | A4265 | 99.01% |
| hH29-41 | ATATCTTCCCAGCTGATAGATG | 4224 | TTTG | A4266 | 99.18% |
| hH29-42 | CAACAATTGGCAATGATGTCAG | 4225 | TTTG | A4267 | 95.26% |

Guides that spanned the intron/exon boundaries were included because such guides may create indels that interfere with splicing. Using Geneious Prime the spacer sequences of these 42 guides were searched against the human genome and a specificity score was assigned by the software based on the alignment to the human genome. A higher specificity score indicates a lower probability of that guide recognizing 1 or more sequences in the human genome other than the site to which the spacer was designed. The specificity scores ranged from 10% to 100% with 25 guides having a specificity score greater than 90% and 33 guides having a specificity score greater than 80%. This analysis demonstrates that guides targeting exonic regions of a human gene with high specificity scores can be readily identified and it is expected that a number of highly active guides are to be identified.

### Example 11—Comparison of the Editing Potency of Nucleases Described Herein to that of spCas9 in Mouse Liver Cells

The CRISPR Cas9 nuclease from the bacterial species Streptococcus pyogenes (spCas9) is widely used for genome editing and is among the most active RNA guided nucleases identified. The relative potency of MG29-1 compared to spCas9 was evaluated by nucleofection of different doses of RNP in the mouse liver cell line Hepa1-6. sgRNA targeting intron 1 of mouse albumin were used for both nucleases. For MG29-1, the sgRNA mA1b29-1-8 identified in Example 29 was selected. Guide mA1b29-1-8 (see Example 29) was chemically synthesized incorporating chemically modifications called AltR1/AltR2 (Integrated DNA Technologies) designed to improve the potency of guides for the Type V nuclease cpf1 that has a similar sgRNA structure as MG29-1. For spCas9 a sgRNA that efficiently edited mouse albumin intron 1 was identified by testing 3 guides selected from an in-silico screen. The spCas9 protein used in these studies was obtained from a commercial supplier (Integrated DNA technologies AltR-sPCas9).

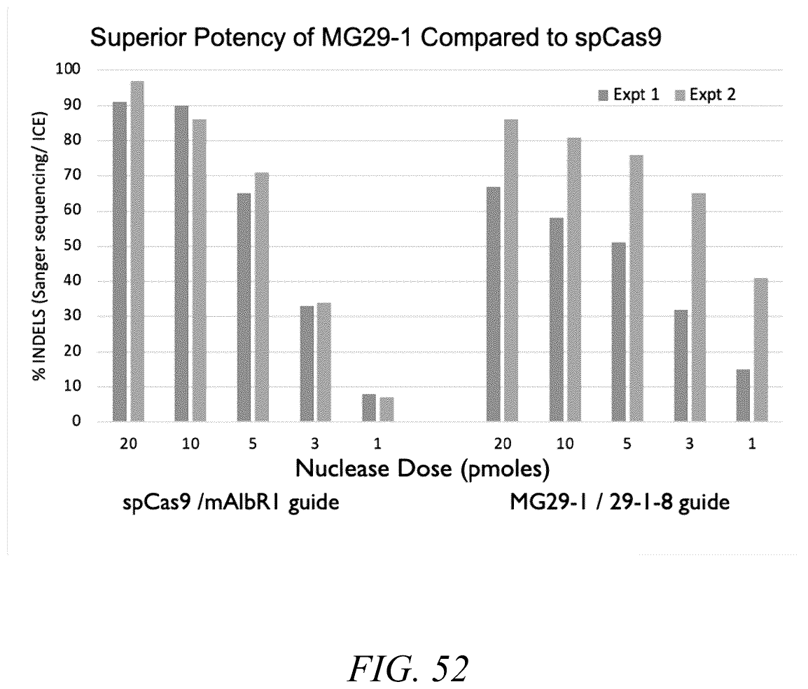

The sgRNA mA1bR1 (spacer sequence TTAGTATAGCATGGTCGAGC) was chemically synthesized and incorporated chemical modifications comprised of 2′ O methyl bases and phosphorothioate (PS) linkages on the 3 bases on both ends of the guide that improve potency in cells. The mA1bR1 sgRNA generated INDELS at a frequency of 90% when RNP comprised of 20 pmol spCas9 protein/50 pmol of guide was nucleofected into Hepa1-6 cells indicating that this is a highly active guide. RNP formed with a range of nuclease protein from 20 pmoles to 1 pmole and a constant ratio of protein to sgRNA of 1:2.5 were nucleofected into Hepa1-6 cells. INDELS at the target site in mouse albumin intron 1 were quantified using Sanger sequencing of the PCR amplified genomic DNA and ICE analysis. The results shown in FIG. 52 demonstrate that MG29-1 generated a higher percentage of INDELS than spCas9 at lower RNP doses when the editing was not saturating. These data indicate that MG29-1 is at least as active and potentially more active than spCas9 in liver-derived mammalian cells.

### Example 34—Engineering Sequence Variants of Nucleases Described Herein and Evaluation in Mouse Liver Cells

In order to improve the editing efficiency of MG29-1 a set of mutations substituting one or two amino acids was introduced in the MG29-1 coding region. The set of amino acid substitutions was determined by alignment to Acidaminococcus sp. Cas12a (AsCas12a). Structured-guide engineering (Kleinstiver, et al, Nat Biotechnol. 2019, 37 276-282) substituted different amino acid in AsCas12a with the goal of altering or improving PAM binding. Four amino acid substitutions in AsCas12a: S170R, E174R, N577R and K583R, showed higher editing efficiencies with canonical and non-canonical PAMs. Sites matching these substitutions were identified in MG29-1 by multiple alignment and correspond to: S168R, E172R, N577R and K583R in MG29-1.

In order to test the single amino acid substitutions a 2-plasmid delivery system was used. Expression plasmids encoding MG29-1 with single amino acid substitutions were constructed using standard molecular cloning techniques. One plasmid encoded for MG29-1 under CMV promoter, the second plasmid contained the mA1b29-1-8 sgRNA (see Table 8), which has high editing efficiency in Hepa 1-6 cells. Transcription of the guide was driven by a human U6 promoter. Confirmation of initial results from single amino acid substitutions using the 2-plasmid system and testing of double amino acid substitutions was done using in vitro transcribed (IVT) mRNA encoding MG29-1 (see Example 11 for details of how the IVT mRNA was made) and chemically synthesized guides incorporating the AltR1/AltR2 chemical modifications that had been optimized by Integrated DNA Technologies for Cpf1 (synthesized at Integrated DNA technologies). For delivery of the 2-plasmid system 100 ng of plasmid encoding MG29-1 and 400 ng of plasmid encoding the guide were mixed with Lipofectamine 3000, added to Hepa1-6 cells and incubated for 3 days before to genomic DNA isolation.

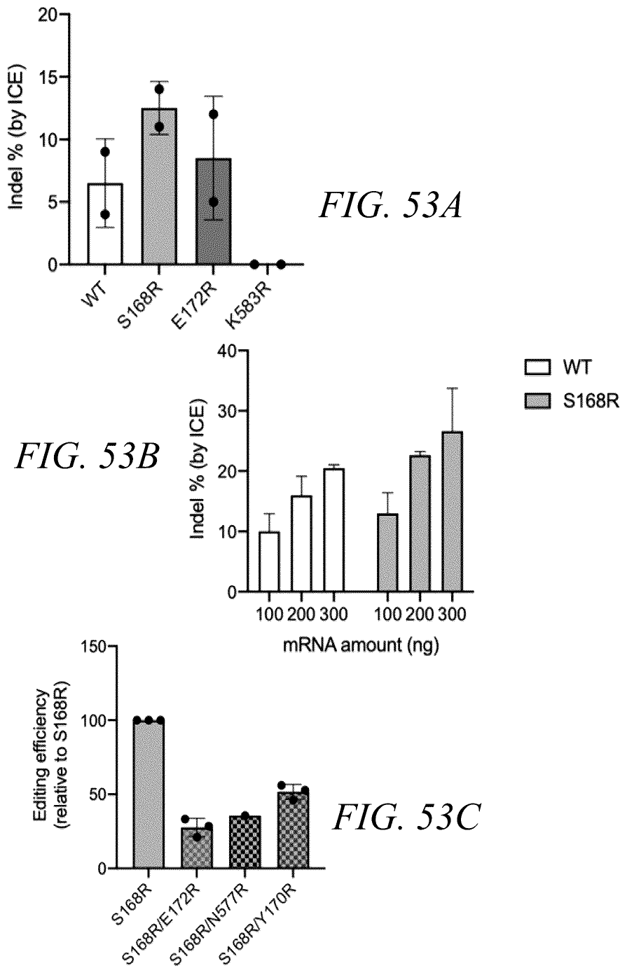

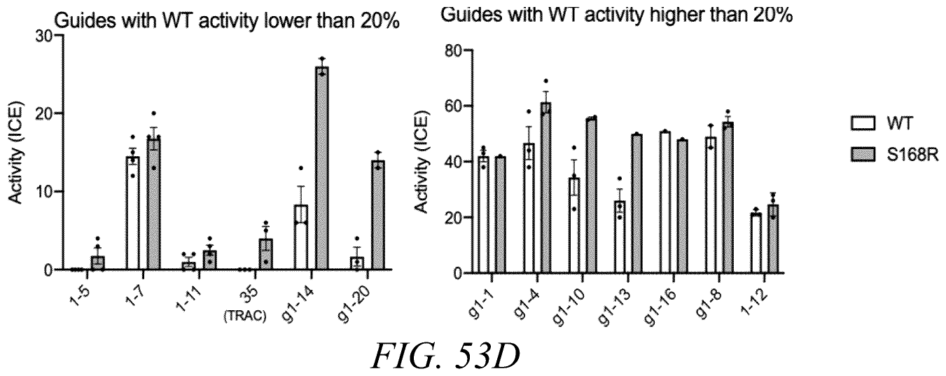

For delivery of IVT mRNA and synthetic guides, 300 ng of mRNA and 120 ng of synthetic guide were mixed with Lipofectamine Messenger Max, added to cells and incubated for 2 days before to genomic DNA isolation. Synthetic guides screened using IVT mRNA correspond to guides detailed in Table 8 but for simplicity the names of the guides in FIG. 53 have been shortened so that guide “mA1b29-1-1” is represented as g1-1, “mA1b29-1-8” is represented as g1-8 and so on. One guide targeting the human T cell receptor locus (TRAC) was also tested (35 TRAC on FIG. 53D). Guide 35 TRAC spacer is: GAGTCTCTCAGCTGGTACACGG (SEQ ID NO: 4268) with a TTTG PAM. Guide 35 TRAC was ordered with the same modifications as mentioned before. Genomic DNA and PCR amplification was performed as described in the previous example for MG29-1 editing of mouse albumin intron 1. For guide 35 TRAC, the human TRAC locus was amplified with Primer F: TGCTTTGCTGGGCCTTTTTC (SEQ ID NO: 4269), Primer R: ACAGTCTGAGCAAAGGCAGG (SEQ ID NO: 4270). The resulting 957 bp PCR product was purified as described previously. Editing was assessed by Sanger sequencing using primer ATCACGAGCAGCTGGTTTTCT (SEQ ID NO: 4271).

Editing efficiency for mouse albumin intron 1 and human TRAC locus was quantified using Sanger sequencing of the PCR products followed by Inference of CRISPR Edits (ICE). Data representing up to 4 biological replicates are plotted in FIGS. 53A-D. The single amino acid substitution S168R demonstrated improved editing efficiency when using guide mA1b29-1-8 in the 2-plasmid system (FIG. 53A). Mutation E172R did not provide a major improvement with guide mA1b29-1-8 while the mutation K583R completely prevented editing with the mA1b29-1-8 guide. Transfection with MG29-1 mRNA and synthetic guide mA1b29-1-8 confirmed the results from plasmid transfection (FIG. 53B). The single amino acid substitution S168R conferred higher editing efficiency across the different concentrations of mRNA tested with guide mA1b29-1-8 (FIG. 53B). The double amino acid substitutions of S168R with E172R (substitution that did not impair activity alone as seen in FIG. 53A), or N577R (a substitution not tested in MG29-1 plasmid transfection but conferred higher editing efficiency of cpf1) and Y170R (which it was hypothesized might improve editing efficiency based on the predicted MG29-1 protein structure) were tested and compared to the single S168R mutant.

None of the double mutations conferred improved editing efficiencies under the conditions tested (FIG. 53C). The editing efficiencies of the S168R variant of MG29-1 and MG29-1 WT were compared in parallel with 12 guides targeting mouse albumin intron 1 and 1 guide targeting the human T cell receptor locus (TRAC). The S168R variant of MG29-1 exhibited improved editing efficiency with all 13 guides with some guides benefiting more than others (FIG. 4d). Importantly S168R did not impair mammalian editing efficiency for any of the guides tested. These results demonstrate that the S168R (serine at amino acid position 168 changed to arginine) variant of MG29-1 has improved editing activity and which is advantageous in identifying highly active guides for therapeutic use.

### Example 35—Identification of Chemical Modifications of the sgRNA of Nucleases Described Herein that Improve Guide Stability and Improve Editing Efficiency in Mammalian Cells

RNA molecules are inherently unstable in biological systems due to their sensitivity to cleavage by nucleases. Modification of the native chemical structure of RNA has been widely used to improve the stability RNA molecules used for RNA interference (RNAi) in the context for therapeutic drug development (Corey, J Clin Invest. 2007 Dec. 3; 117(12): 3615-3622, J. B. Bramsen, J. Kjems Frontiers in Genetics, 3 (2012), p. 154). The introduction of chemical modifications to the nucleobases or the phosphodiester backbone of RNA have been pivotal in improving the stability and thus the potency of short RNA molecules in vivo. A wide range of chemical modifications with different properties in terms of stability against nucleases and affinity to complementary DNA or RNA have been developed.

Similar chemical modifications have been applied to the guide RNA for CRISPR Cas9 nucleases (Hendel et al, Nat Biotechnol. 2015 September; 33(9): 985-989, Ryan et al Nucleic Acids Res 2018 Jan. 25;46(2):792-803., Mir et al Nature Communications volume 9, Article number: 2641 (2018), O'Reilly et al Nucleic Acids Res 2019 47, 546-558, Yin et al Nature Biotechnology volume 35, pages 1179-1187(2017), each of which is incorporated by reference herein in its entirety).

The MG29-1 nuclease is a novel nuclease with limited amino acid sequence similarity to identified Type V CRISPR enzymes such as cpf1. While the sequence of the structural (backbone) component of the guide RNA identified for MG29-1 is similar to that of cpf1 chemical modifications to the MG29-1 guide that enable improved stability while retaining activity had not been identified. A series of chemical modifications of the MG29-1 sgRNA were designed in order to evaluate their impact on sgRNA activity in mammalian cells and stability in the presence of mammalian cell protein extracts.

We selected the sgRNA mA1b29-1-8 which was highly active in the mouse liver cell line Hepa1-6 when the guide contained a set of proprietary chemical modifications developed by IDT called AltR1/AltR2 that were designed to improve the activity of the guide RNA for cpf1 and are available commercially (IDT). We selected to test 2 chemical modifications of the nucleobase; 2′-O-Methyl in which the 2′ hydroxyl group is replaced with a methyl group, and 2′-fluoro in which the 2′ hydroxyl group is replaced with a fluorine. Both 2′-O-Methyl and 2′-fluoro modifications improve resistance to nucleases. The 2′-O-methyl modification is a naturally occurring post-transcriptional modification of RNA and improves the binding affinity of RNA:RNA duplexes but has little impact on RNA:DNA stability. 2′-fluoro modified bases have reduced immunostimulatory effects and increase the binding affinity of both RNA: RNA and RNA:DNA hybrids (see e.g. Pallan et al Nucleic Acids Res 2011 Apr; 39(8):3482-95, Chen et al Scientific Reports volume 9, Article number: 6078 (2019), Kawasaki, A. M. et al. J Med Chem 36, 831-841 (1993)).

The inclusion of phosphorothioate (PS) linkages in place of phosphodiester linkages between the bases was also evaluated. PS linkages improve resistance to nucleases (Monia et al Nucleic Acids, Protein Synthesis, and Molecular Genetics| Volume 271, ISSUE 24, P14533-14540, Jun. 14, 1996).

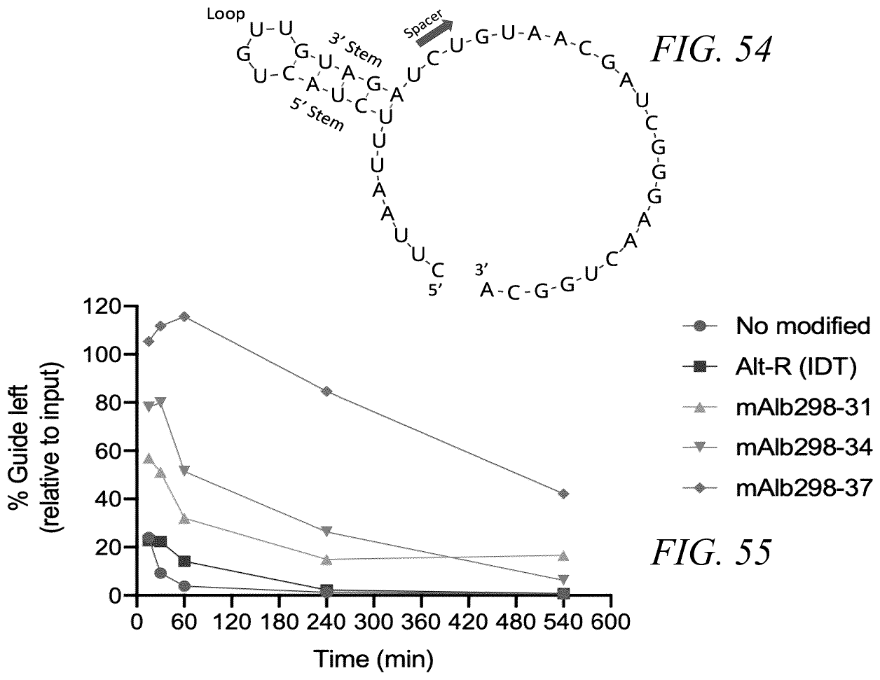

The predicted secondary structure of the MG29-1 sgRNA with the spacer targeting mouse albumin intron 1 (mA1b29-1-8) is shown in FIG. 54. The stem-loop in the backbone portion of the guide was presumed to be critical for interaction with the MG29-1 protein based on sequence organization of other CRISPR-cas systems. Based on the secondary structure a series of chemical modifications was designed in different structural and functional regions of the guide. A modular approach was taken that allowed initial testing of guides with fewer chemical modifications that inform which structural and functional regions of the guide may tolerate different chemical modifications without significant loss of activity. The structural and functional regions were defined as follows. The 3′ end and 5′ end of the guide are targets for exonucleases and can be protected by various chemical modifications including 2′-O-methyl and PS linkages, an approach that has been used to improve the stability of guides for spCas9 (Hendel et al, Nat Biotechnol. 2015 September; 33(9): 985-989).

The sequences comprising both halves of the stem and the loop in the backbone region of the guide were selected for modification. The spacer was divided into the seed region (first 6 nucleotides closest to the PAM) and the remaining 16 nucleotides of the spacer (referred as the non-seed region). In total 43 guides were designed and 39 were synthesized. All 43 guides contain the same nucleotide sequence but with different chemical modifications. The editing activity of 39 of the guides was evaluated in Hepa1-6 cells by nucleofection of RNP or by co-transfection of mRNA encoding MG29-1 and guide or by both methods. These two methods of transfection may impact the observed activity of the guide due to differences in the delivery to the cell.

When nucleofection of a RNP is used the guide and the MG29-1 protein are pre-complexed in a tube and then delivered to the cell using nucleofection in which an electric current is applied to the cells' suspension in the presence of the RNP. The electric current transiently opens pores in the cell membrane (and possibly the nuclear membrane as well) enabling cellular entry of the RNP driven by the charge on the RNP. Whether the RNP enters the nucleus via pores created by the electric current or via the nuclear localization signals engineered in the protein component of the RNP, or a combination of the two is unclear.

When co-transfection of mRNA and guide with a lipid transfection reagent such as Messenger MAX is used, the mixture of the two RNA forms a complex with the positively charged lipid and the complex enters the cells via endocytosis and eventually reaches the cytoplasm. In the cytoplasm the mRNA is translated into protein. In the case of an RNA guided nucleases such as MG29-1 the resulting MG29-1 protein will presumably form a complex with the guide RNA in the cytoplasm before entering the nucleus in a process mediated by the nuclear localization signals that were engineered into the MG29-1 protein.

Because translation of the mRNA into sufficient amounts of MG29-1 protein followed by the binding of the MG29-1 protein to the guide RNA takes a finite amount of time, the guide RNA may require increased stability in the cytoplasm for longer than is the case when pre-formed RNP is delivered by nucleofection. Thus lipid-based mRNA/sgRNA co-transfection may require a more stable guide than is the case for RNP nucleofection which may result in some guide chemistries being active as RNP but inactive when co transfected with mRNA using cationic lipid reagents.

Guides mA1b298-1 to mA1b298-5 contain chemical modifications limited to the 5′ and 3′ ends of the sequence using a mixture of 2′-O-methyl and 2′ fluoro bases plus PS linkages. In comparison to the sgRNA without chemical modifications these guides were 7 to 11-fold more active when delivered via RNP demonstrating that end modifications to the guide improved guide activity, presumably through improved resistance to exonucleases. sgRNA mA1b298-1 to mA1b298-5 exhibited 64 to 114% of the editing activity of the guide containing the commercial chemical modifications (A1tR1/A1tR2). Guide 4, which contains the largest number of chemical modifications, was the least active of the end modified guides but was still 7-fold more active than the un-modified guide. Guide mALB298-30 contains three 2′-O methyl bases and 2 PS linkages at the 5′ end and 4 2′-O methyl bases and 3 PS linkages at the 5′ end and also exhibited activity about 10-fold higher than the unmodified guide and similar or slightly improved in the case of RNA co-transfection compared to mA1b298-1. These data demonstrate that 2′O-methyl combined with PS linkages on both ends of the MG29-1 guide significantly enhanced guide activity compared to an unmodified guide.

A combination of 2′-fluoro bases and PS linkages were also tolerated at the 3′ end of the guide. Guide mALb298-28 contains three 2′-fluoro bases and 2 PS linkages on the 5′ end and four 2′-fluoro bases and three PS linkages on the 3′ end. This end modified guide retained good editing activity similar to the guides with 2′-O methyl and PS modifications on both ends demonstrating that 2′-fluoro can be used in place of 2′-O methyl to improve guide stability and retain editing activity.

The sgRNAs mALb298-6, mALb298-7, and mALb298-8 contain the same minimal chemical modifications on the both 5′ and 3′ ends present in mA1b298-1 plus PS linkages in different regions of the stem. PS linkages in the 3′ stem (mALb298-6) and the 5′ stem (mALb298-7) reduced activity by about 30% compared to mA1b298-1 in the RNP nucleofection assay, indicating that these modifications may be tolerated. Larger reductions in activity were observed by lipid-based transfection.

Introducing PS linkages in both the 3′ and 5′ stems (mALb298-8) reduced activity by about 80% compared to mA1b298-1 in the RNP nucleofection assay and by more than 95% in the lipid transfection assay, indicating that the combination of two PS linkage modifications significantly impaired the function of the guide.

The sgRNA mA1b298-9 contains the same minimal chemical modifications on the both 5′ and 3′ ends present in mA1b298-1 plus PS linkages in the loop and exhibited similar activity as mA1b298-1 indicating that PS linkages in the loop were well tolerated.

The sgRNAs mA1b298-10, mA1b298-11, and mA1b298-12 contain the same minimal chemical modifications on the both 5′ and 3′ ends present in mA1b298-1 plus 2′-O methyl bases in different regions of the stem. Including 2′-O methyl bases in either the 3′ stem (mA1b298-11) or the 5′ stem (mA1b298-12) or both halves of the stem (mA1b298-10) was generally well tolerated with small reductions in activity compared to mA1b298-1 with guide mA1b298-12 (5′ stem modified) being the most active.

Guide mA1b298-14 contains the same minimal chemical modifications on the both 5′ and 3′ ends present in mA1b298-1 plus a combination of 2′-O-methyl bases and PS linkages in both halves of the stem and had no editing activity by RNP nucleofection or by lipid-based RNA co-transfection. This confirms and extends the result with mA1b298-8 that contained only PS linkages in both stems had retained low levels of activity and shows that extensive chemical modification of both halves of the stem makes the guide inactive.

The sgRNA mA1b298-13 contains the same minimal chemical modifications on the both 5′ and 3′ ends present in mA1b298-1 plus PS linkages spaced every other base throughout the remainder of the backbone and spacer except for in the seed region of the spacer. These modifications resulted in a dramatic loss of editing activity to close to background levels. While the purity of this guide was about 50% compared to >75% for most of the guides, this alone may not account for the complete loss of editing activity. Thus, distributing PS linkages in an essentially random fashion throughout the guide is not an effective approach to improve guide stability while retaining editing activity.

Guides mALb298-15 and mALb298-16 contain the same minimal chemical modifications on the both 5′ and 3′ ends present in mA1b298-1 plus extensive PS linkages in the backbone. While both guides retained about 35% of the activity of mA1b298-1 by RNP nucleofection they retained 3% of the activity of mA1b298-1 by lipid-based RNA co-transfection indicating that extensive PS modification of the backbone significantly reduced editing activity. Combining the PS linkages in the backbone with PS linkages in the spacer region as in mA1b298-17 and mA1b298-18 resulted in further loss of activity consistent with the observation the random inclusion of PS linkages is blocks the ability of the guide to direct editing by MG29-1.

Guide mA1b298-19 contains the same chemical modifications in the spacer as mALb298-1 but in the backbone region the 5′ end has additional 4 2′O-methyl bases and an additional 14 PS linkages. The activity of mA1b298-19 was about 40% of that of mA1b298-1 by RNP nucleofection but 22% by RNA co-transfection demonstrating again that extensive chemical modifications in the backbone region of the guide are not well tolerated.

Guides mA1b298-20, mA1b298-21, mA1b298-22, and mA1b298-23 have identical chemical modifications in the backbone region comprised of a single 2′-O methyl and 2 PS linkages at the 5′ end which are the same 5′ end modifications as in mA1b298-1. The spacer regions of Guides mA1b298-20, mA1b298-21, mA1b298-22, and mA1b298-23 contain combinations of 2′-O-methyl and 2′-fluoro bases as well as PS linkages. The most active of these 4 guides was mA1b298-2 in which 2′-fluoro modifications were made on all bases in the spacer except for the 7 bases closest to the PAM (seed region) and the terminal base at the 3′ end which was modified with a 2′-O-methyl and 2 PS linkages. This demonstrates that including 2′-fluoro modifications on most of the spacer except for the seed region did not significantly reduce activity and thus represents a good strategy to enhance guide stability.

Guides mA1b298-24, mA1b298-25, mA1b298-26, and mA1b298-8 have identical chemical modifications in the backbone. mA1b298-8 which has PS linkages in both halves on the stem had significantly reduced editing activity with 24% and 2% of guide mA1b298-1 demonstrating that these PS linkages impaired activity. Interestingly, while mALb298-24 and mALb298-25 also had low editing activity the activity of mALb298-26 was improved compared to mA1b298-8 indicating that the additional modifications in mALb298-26 which comprise 2′-fluoro bases in 14 of the bases in the spacer (excluding the seed region) at least partially rescued the reduced editing activity caused by the PS linkages in the stem. This provides additional evidence of the beneficial impact of 2′-fluoro bases in the spacer upon editing activity.

Guides mA1b298-27 and mA1b298-29 contain extensive base and PS modifications throughout the backbone and spacer regions had no activity again indicating that not all chemical modifications of the guide retain editing activity.

Based on the structure activity relationships obtained from the analysis of guides mALb298-1 to mALb298-30, an additional set of seven guides were designed and tested by RNP nucleofection and lipid-based RNA co-transfection of Hepa1-6 cells. These guides combined chemical modifications that were observed to retain good editing activity in guides mALb298-1 to mALb298-30. Guides mALb298-31 to mALb298-37 all contain end modifications comprised of at least one 2′-O methyl and 2 PS linkages at the 5′ end and one 2′-O methyl and 1 PS linkage at the 5′ end. In addition to the end modifications, combining 2′-O methyl bases in both halves of the stem with 2′fluoro bases in 14 bases of the spacer (excluding the seed region) as in mA1b298-31 resulted in editing activity that was slightly improved or similar to end modifications alone and 10-fold improved compared to the unmodified guide. Combining 2′-O methyl bases in just the 5′ stem with 2′fluoro bases in 14 bases of the spacer (excluding the seed region) as in mALb298-32 resulted in a guide that was among the most active tested.

Similarly, combining PS linkages in just the loop with 2′fluoro bases in 14 bases of the spacer (excluding the seed region) as in mALb298-33 resulted in potent activity up to 15-fold higher than the unmodified guide. Guide mA1b298-37 combines more extensive 3′ end modifications with 2′-O methyl bases in the 5′ stem, PS linkages in the loop and 14 2′fluoro bases and 3 PS linkages in the spacer (excluding the seed region) and still retained editing activity similar to that of the AltR1/R2 modifications and significantly improved compared to the unmodified guide. mALb298-37 thus represents a heavily modified MG29-1 guide that retains potent editing activity in mammalian cells. Guide mALb298-38 exhibited potent editing activity when delivered as a RNP but was completely inactive when delivered to cells by lipid-based RNA co-transfection suggesting that thus guide may have some unexpected sensitivity to nucleases. Guide mALb298-39 which is identical to guide mA1b298-37 except that it has 11 fewer 2′-fluoro bases and 1 less PS linkage in the spacer had the highest editing activity when considering both RNP and mRNA transfection methods but has fewer chemical modifications than some of the other guide designs which might be detrimental in terms of performance in vivo.

Additional combinations of chemical modifications were designed to create mA1b298-40 to mALb298-43 that might also retain good editing activity while having more extensive chemical modifications. For example, in mA1b298-41 which also incorporates some DNA bases, 6 of the bases are un-modified ribonucleotides. Similarly, mA1b298-42 contains 2′-fluorogroups throughout the entire spacer and has 5 un-modified ribonucleotides. We envisage that testing of these and other guide chemical modifications will lead to one or more optimized designs. Nevertheless, within the set of guides mALb298-1 to mALb298-39 and particularly among the set of guides mALb298-31 to mALb298-39 we have identified guides with extensive chemical modifications that retain editing activity similar or superior to that of unmodified guides or guides with just end modifications.

In order to test the stability of the chemically modified guides compared to the guide with no chemical modification (native RNA), a stability assay using cell crude extracts was used. Crude cell extracts from mammalian cells were selected because they should contain the mixture of nucleases that a guide RNA will be exposed to when delivered to mammalian cells in vitro or in vivo. Hepa 1-6 cells were collected by adding 3 ml of cold PBS per 15 cm dish of confluent cells and releasing the cells from the surface of the dish using a cell scraper. The cells were pelleted at 200 g for 10 min and frozen at −80° C. for future use. For the stability assays, cells were resuspended in 4 volumes of cold PBS (e.g. for a 100 mg pellet cells were resuspended in 400 μl of cold PBS). Triton X-100 was added to a ending concentration of 0.2% (v/v), cells were vortexed for 10 seconds, put on ice for 10 minutes and vortexed again for 10 seconds. Triton X-100 is a mild non-ionic detergent that disrupts cell membranes but does not inactivate or denature proteins at the concentration used.

Stability reactions were set up on ice and comprised 20 μl of cell crude extract with 100 fmoles of each guide (1 μl of a 100 nM stock). Six reactions were set up per guide comprising: input, 15 min, 30 min, 60 min, 240 min and 540 min (The time in minutes referring to the length of time each sample was incubated). Samples were incubated at 37° C. from 15 minutes up to 540 min while the input control was left on ice for 5 minutes. After each incubation period the reaction was stopped by adding 300 μl of a mixture of phenol and guanidine thiocyanate (Tri reagent, Zymo Research) which immediately denatures all proteins and efficiently inhibits ribonucleases and facilitates the subsequent recovery of RNA. After adding Tri Reagent the samples were vortexed for 15 seconds and stored at −20° C. RNA was extracted from the samples using Direct-zol RNA miniprep kit (Zymo Research) and eluted in 100 μl of nuclease-free water. Detection of the modified guide was performed using Taqman RT-qPCR using the Taqman miRNA Assay technology (Thermo Fisher) and primers and probes designed to specifically detect the sequence in the mA1b298 sgRNA which is the same for all of the guides. Data was plotted as a function of percentage of sgRNA remaining in relation to the input sample. The guide with no chemical modifications was the most rapidly eliminated when incubated with the cell extract (FIG. 55) with more than 90% of the guide degraded within 30 minutes. The guide with the AltR1/AltR2 (AltR in FIG. 55) chemical modifications was slightly more stable in the presence of cell extract than the un-modified guide with about 80% of the guide degraded in 30 minutes. Guide mALb298-31 that contains chemical modifications at both ends as well as 2′ O-methyl bases in both stems and 2′-fluoro bases at all positions of the spacer except for the seed region was significantly more stable than either unmodified guide or the AltR guide.

Guide mA1b298-34 exhibited improved stability compared to guide mALb298-31. Guide mALb298-34 differs to guide mALb298-31 in the chemical modifications within the spacer. mALb298-34 has 9 fewer 2′-Fluoro bases in the spacer than mALb298-31 but contains 4 PS linkages in the spacer compared to 2 PS linkages in mALb298-31. Because 2′-fluoro bases improve the stability of RNA this suggests that the additional PS linkages in the spacer were responsible for the improved stability of mALb298-34 compared to mALb298-31.

Guide mALb298-37 was the most stable of all the guides tested and was significantly more stable than mALb298-34 with 80% of the guide remaining after 240 min (4 h) compared to 30% for mALb298-34. The chemical modifications of mALb298-37 differ from guide mALb298-34 in both the spacer and backbone regions. mALb298-37 has an additional two 2′-O-methyl groups and 2 additional PS linkages at the 5′ end. In addition, the loop region of mALb298-37 contains PS linkages and does not contain the 2′-O-methyl groups present in the second half of the stem in mALb298-34. In addition, the spacer of mALb298-37 contains 9 more 2′-fluoro bases but the same number of PS linkages as mALb298-34 albeit in different locations.

Overall, these data suggest that additional PS linkages at the 5′ end of the spacer and in the loop of the backbone region significantly improve stability of the guide RNA. Guide mALb298-37 which exhibited the greatest stability in the cell extracts among the guides tested also exhibited potent editing activity in Hepa1-6 cells that was similar or improved compared to the AltR1/Altr2 modifications and improved compared to chemical modifications of the 5′ and 3′ ends only.

TABLE 5
Impact of chemical modifications of the MG29-1 sgRNA sequence upon editing
activity in mammalian cells

| | | | Editing activity | |
|---|---|---|---|---|
| | | | (% of | |
| | | | AltR1/AltR2 | |
| | | SEQ ID | control) | |
| sgRNA name | sgRNA sequence | NO: | RNP | mRNA |
| mAlb298-1_ | /AltR1/rCrUrUrArArUrUrUrCrUrArCrUr | N/4272 | 100 | 100 |
| AltR1/R2 | GrUrUrGrUrArGrArUrCrUrGrUrArArCrGr | | | |
| | ArUrCrGrGrGrArArCrUrGrGrCrA/AltR2/ | | | |
| mAlb298-0 | rCrUrUrArArUrUrUrCrUrArCrUrGrUrUrG | 4272 | 13.5 | NT |
| | rUrArGrArUrCrUrGrUrArArCrGrArUrCrG | | | |
| | rGrGrArArCrUrGrGrCrA | | | |
| mAlb298-1 | mC*rU*rUrArArUrUrUrCrUrArCrUrGrUrU | 4273 | 114.7 | 76.2 |
| | rGrUrArGrArUrCrUrGrUrArArCrGrArUrC | | | |
| | rGrGrGrArArCrUrGrG*rC*mA | | | |
| mAlb298-2 | mC*rU*rU*rArArUrUrUrCrUrArCrUrGrUr | 4274 | 111.7 | 70.2 |
| | UrGrUrArGrArUrCrUrGrUrArArCrGrArUr | | | |
| | CrGrGrGrArArCrUrG*rG*rC*mA | | | |
| mAlb298-3 | mC*mU*rU*rArArUrUrUrCrUrArCrUrGrUr | 4275 | 100.2 | 63.7 |
| | UrGrUrArGrArUrCrUrGrUrArArCrGrArUr | | | |
| | CrGrGrGrArArCrUrG*rG*mC*mA | | | |
| mAlb298-4 | mC*mU*mU*rArArUrUrUrCrUrArCrUrGrUr | 4276 | 72.5 | 69.6 |
| | UrGrUrArGrArUrCrUrGrUrArArCrGrArUr | | | |
| | CrGrGrGrArArCrUrG*mG*mC*mA | | | |
| mAlb298-5 | mC*rU*rUrArArUrUrUrCrUrArCrUrGrUrU | 4277 | 76.9 | 87.5 |
| | rGrUrArGrArUrCrUrGrUrArArCrGrArUrC | | | |
| | rGrGrGrArArCrUrG*/12FG//i2FC/*/32F | | | |
| | A/ | | | |
| mAlb298-6 | mC*rU*rUrArArUrUrUrCrUrArCrUrGrUrU | 4278 | 89.4 | 40.4 |
| | rG*rU*rA*rG*rArUrCrUrGrUrArArCrGrA | | | |
| | rUrCrGrGrGrArArCrUrGrG*rC*mA | | | |
| mAlb298-7 | mC*rU*rUrArArUrU*rU*rC*rU*rArCrUrG | 4279 | 83.2 | 24.5 |
| | rUrUrGrUrArGrArUrCrUrGrUrArArCrGrA | | | |
| | rUrCrGrGrGrArArCrUrGrG*rC*mA | | | |
| mAlb298-8 # | mC*rU*rUrArArUrU*rU*rC*rU*rArCrUrG | 4280 | 28.4 | 2.6 |
| | rUrUrG*rU*rA*rG*rArUrCrUrGrUrArArC | | | |
| | rGrArUrCrGrGrGrArArCrUrGrG*rC*mA | | | |
| mAlb298-9 | mC*rU*rUrArArUrUrUrCrUrArCrU*rG*rU | 4281 | 110.9 | 87.5 |
| | *rU*rGrUrArGrArUrCrUrGrUrArArCrGrA | | | |
| | rUrCrGrGrGrArArCrUrGrG*rC*mA | | | |
| mAlb298-10 | mC*rU*rUrArArUrUmUmCmUmArCrUrGrUrU | 4282 | 87.5 | 61.6 |
| | rGmUmAmGmArUrCrUrGrUrArArCrGrArUrC | | | |
| | rGrGrGrArArCrUrGrG*rC*mA | | | |
| mAlb298-11 | mC*rU*rUrArArUrUrUrCrUrArCrUrGrUrU | 4283 | 94.5 | 63.5 |
| | rGmUmAmGmArUrCrUrGrUrArArCrGrArUrC | | | |
| | rGrGrGrArArCrUrGrG*rC*mA | | | |
| mAlb298-12 | mC*rU*rUrArArUrUmUmCmUmArCrUrGrUrU | 4284 | 121.8 | 84.0 |
| | rGrUrArGrArUrCrUrGrUrArArCrGrArUrC | | | |
| | rGrGrGrArArCrUrGrG*rC*mA | | | |
| mAlb298-13 # | mC*rU*rU*rArA*rUrU*rUrC*rUrA*rCrU* | 4285 | 1.0 | 0.0 |
| | rGrUrUrG*rUrA*rGrA*rUrCrUrGrUrArA | | | |
| | *rCrG*rArU*rCrG*rGrG*rArA*rCrU*rGr | | | |
| | G*mC*mA | | | |
| mAlb298-14 | mC*rU*rUrArArUrU*mU*mC*mU*mArCrUrG | 4286 | 0.0 | 0.0 |
| | rUrUrG*mU*mA*mG*mArUrCrUrGrUrArArC | | | |
| | rGrArUrCrGrGrGrArArCrUrGrG*rC*mA | | | |
| mAlb298-15 # | mC*rU*rU*rArArU*rU*rU*rC*rU*rA*rC* | 4287 | 39.3 | 2.4 |
| | rU*rG*rU*rU*rG*rU*rA*rG*rA*rUrCrUr | | | |
| | GrUrArArCrGrArUrCrGrGrGrArArCrUrGr | | | |
| | G*rC*mA | | | |
| mAlb298-16 | mC*rU*rU*rArArU*rU*rU*rC*rUrArCrU* | 4288 | 41.6 | 3.7 |
| | rG*rU*rU*rG*rU*rA*rG*rA*rUrCrUrGrU | | | |
| | rArArCrGrArUrCrGrGrGrArArCrUrGrG*r | | | |
| | C*mA | | | |
| mAlb298-17 # | mC*rU*rU*rArArU*rU*rU*rC*rUrArCrU* | 4289 | 0.0 | 1.2 |
| | rG*rU*rU*rG*rU*rA*rG*rA*rUrCrUrGrU | | | |
| | rArA*rCrG*rArU*rCrG*rGrG*rArA*rCrU | | | |
| | *rGrG*mC*mA | | | |
| mAlb298-18 | mC*rU*rU*rA*rA*rU*rU*rU*rC*rU*rA*r | 4290 | 5.2 | 1.2 |
| | C*rU*rG*rU*rU*rG*rU*rA*rG*rA*rUrCr | | | |
| | UrGrUrArA*rCrG*rArU*rCrG*rGrG*rArA | | | |
| | *rCrU*rGrG*mC*mA | | | |
| mAlb298-19 | mG*mU*mA*mG*mC*rU*rU*rArA*rUrU*rUr | 4291 | 50.1 | 17.4 |
| | C*rUrA*rCrU*rGrU*rUrG*rUrA*rGrA*rU | | | |
| | rCrUrGrUrArArCrGrArUrCrGrGrGrArArC | | | |
| | rUrGrG*rC*mA | | | |
| mAlb298-20 | mC*rU*rUrArArUrUrUrCrUrArCrUrGrUrU | 4292 | 33.6 | 86.3 |
| | rGrUrArGrArUrCrUrGrUrArArCrGrArUrC | | | |
| | rGrGrGrArArC*/12FU//i2FG/*/12FG//i | | | |
| | 2FC/*/32FA/ | | | |
| mAlb298-21 | mC*rU*rUrArArUrUrUrCrUrArCrUrGrUrU | 4293 | 119.0 | 80.6 |
| | rGrUrArGrArUrCrUrGrUrArArC/i2FG//i | | | |
| | 2FA//12FU//12FC//12FG//12FG//12FG/ | | | |
| | /12FA//12FA//12FC//12FU//12FG//12F | | | |
| | G/*/12FC/*mA | | | |
| mAlb298-22 | mC*rU*rUrArArUrUrUrCrUrArCrUrGrUrU | 4294 | 25.1 | 98.8 |
| | rGrUrArGrArUrCrUrGrUrArArCrGrArUrC | | | |
| | rGrGrGrA*rA/i2FC/*/12FU//12FG/*/12 | | | |
| | FG//12FC/*mA | | | |
| mAlb298-23 | mC*rU*rUrArArUrUrUrCrUrArCrUrGrUrU | 4295 | 22.6 | 61.9 |
| | rGrUrArGrArUrCrUrGrUrArArCrGrArUrC | | | |
| | rGrGrGrA/i2FA//12FC//12FU//12FG//i | | | |
| | 2FG/*mC*mA | | | |
| mAlb298-24 | mC*rU*rUrArArUrU*rU*rC*rU*rArCrUrG | 4296 | 7.4 | 12.2 |
| | rUrUrG*rU*rA*rG*rArUrCrUrGrUrArArC | | | |
| | rGrArUrCrGrGrGrArArC*/12FU//12FG/* | | | |
| | /12FG//12FC/*/32FA/ | | | |
| mAlb298-25 | mC*rU*rUrArArUrU*rU*rC*rU*rArCrUrG | 4297 | 0.0 | 0.0 |
| | rUrUrG*rU*rA*rG*rArUrCrUrGrUrArArC | | | |
| | rGrArUrCrGrG/12FG//12FA/*/12FA//12 | | | |
| | FC/*/12FU//i2FG/*rGrC*mA | | | |
| mAlb298-26 | mC*rU*rUrArArUrU*rU*rC*rU*rArCrUrG | 4298 | 55.5 | 29.8 |
| | rUrUrG*rU*rA*rG*rArUrCrUrGrUrArArC | | | |
| | /12FG//12FA//12FU//12FC//12FG//12F | | | |
| | G//12FG//12FA//12FA//12FC//12FU//i | | | |
| | 2FG//12FG/*/12FC/*mA | | | |
| mAlb298-27 | /52FC/*/12FU/*/12FU/*rUrUrArArU/i2 | 4299 | NT | 0 |
| | FU//12FU/rC*rU/i2FA/*/12FC/rU/i2FG | | | |
| | /*/12FU//12FU/rG/12FU//12FA//12FG/ | | | |
| | /i2FA/rU/i2FC/rUrG*rUrA/i2FA/rc/i2 | | | |
| | FG/*/12FA/*/12FU/*/12FC/*/12FG/rG* | | | |
| | rGrA*rA/i2FC/*rU*/12FG//12FG/*/12F | | | |
| | C/*/32FA | | | |
| mAlb298-28 | /52FC/*/12FU/*/12F/rUrUrUrArArUrUr | 4300 | NT | 84.8 |
| | UrCrUrArCrUrGrUrUrGrUrArGrArUrCrUr | | | |
| | GrUrArArCrGrArUrCrGrGrGrArArCrU*/i | | | |
| | 2FG//12FG/*/12FC/*/52FA/ | | | |
| mAlb298-29 | mC*mU*mU*rUrUrArArUmUmUrC*rUmA*mCr | 4301 | 0.0 | 0.0 |
| | UmG*mUmUrGmUmAmGmArUmCrUrG*rUrAmAr | | | |
| | CmG*mA*mU*mC*mGrG*rGrA*rAmC*rU*mGm | | | |
| | G*mC*mA | | | |
| mAlb298-30 | mC*mU*mUrUrUrArArUrUrUrCrUrArCrUrG | 4302 | 101.1 | 105.4 |
| | rUrUrGrUrArGrArUrCrUrGrUrArArCrGrA | | | |
| | rUrCrGrGrGrArArCrU*mGmG*mC*mA | | | |
| mAlb298-31 | mC*rU*rUrArArUrUmUmCmUmArCrUrGrUrU | 4303 | 140.5 | 74.4 |
| | rGmUmAmGmArUrCrUrGrUrArArC/i2FG//i | | | |
| | 2FA//12FU//12FC//12FG//12FG//12FG/ | | | |
| | /12FA//12FA//12FC//12FU//12FG//12F | | | |
| | G/*/12FC/*mA | | | |
| mAlb298-32 | mC*rU*rUrArArUrUmUmCmUmArCrUrGrUrU | 4304 | 170.3 | 93.1 |
| | rGrUrArGrArUrCrUrGrUrArArC/i2FG//i | | | |
| | 2FA//12FU//12FC//12FG//12FG//12FG/ | | | |
| | /12FA//12FA//12FC//12FU//12FG//12F | | | |
| | G*/12FC/*mA | | | |
| mAlb298-33 | mC*rU*rUrArArUrUrUrCrUrArCrU*rG*rU | 4305 | 202.7 | 64.4 |
| | *rU*rGrUrArGrArUrCrUrGrUrArArC/i2F | | | |
| | G//12FA//12FU//12FC//12FG//12FG//i | | | |
| | 2FG//12FA//12FA//12FC//12FU//12FG/ | | | |
| | /12FG/*/12FC/*mA | | | |
| mAlb298-34 | mC*rU*rUrArArUrUmUmCmUmArCrUrGrUrU | 4306 | 83.8 | 107.0 |
| | rGmUmAmGmArUrCrUrGrUrArArCrGrArUrC | | | |
| | rGrGrGrA*rA/i2FC/*/12FU//12FG/*/12 | | | |
| | FG//12FC/*mA | | | |
| mAlb298-35 | mC*rU*rUrArArUrUrUrCrUrArCrU*rG*rU | 4307 | 24.3 | 67.9 |
| | *rU*rGrUrArGrArUrCrUrGrUrArArCrGrA | | | |
| | rUrCrGrGrGrA*rA/i2FC/*/12FU//12FG/ | | | |
| | */12FG//12FC/*mA | | | |
| mAlb298-36 | mC*rU*rUrArArUrUmUmCmUmArCrUrGrUrU | 4308 | 43.2 | 116.2 |
| | rGrUrArGrArUrCrUrGrUrArArCrGrArUrC | | | |
| | rGrGrGrA*rA/i2FC/*/12FU//12FG/*/12 | | | |
| | FG//12FC/*mA | | | |
| mAlb298-37 | mC*mU*mU*U*rUrArArUrUmUmCmUmArCrU* | 4309 | 164.9 | 84.5 |
| | rG*rU*rU*rGrUrArGrArUrCrUrGrUrArAr | | | |
| | C/12FG//12FA//12FU//12FC//12FG//12 | | | |
| | FG//12FG//12FA//12FA//12FC/*/12FU/ | | | |
| | */12FG//12FG/*/12FC/*mA | | | |
| mAlb298-38 | mC*mU*mU*rU*rUrArArUrUmUmCmUmArCrU | 4310 | 140.5 | 0.0 |
| | *rG*rU*rU*rGmUmAmGmArUrCrUrGrUrArA | | | |
| | rC/12FG//12FA//12FU//12FC//12FG//i | | | |
| | 2FG//12FG//12FA//12FA//12FC/*/12FU | | | |
| | /*/12FG//12FG/*/12FC/*mA | | | |
| mAlb298-39 | mC*mU*mU*rU*rUrArArUrUmUmCmUmArCrU | 4311 | 135.1 | 114.0 |
| | *rG*rU*rU*rGrUrArGrArUrCrUrGrUrArA | | | |
| | rCrGrArUrCrGrGrGrArArCrU*/12FG//i2 | | | |
| | FG/*/12FC/*mA | | | |
| mAlb298-40 | mC*mU*mU*U*UAAUUmUmCmUmACU*G*U*U*G | 4312 | NT | NT |
| | UAGAU/12FC//12FU//12FG//12FU//i2FA | | | |
| | //12FA//12FC//12FG//12FA//12FU//12 | | | |
| | FC//12FG//12FG//12FG//12FA//12FA// | | | |
| | 12FC/*/12FU/*/12FG//12FG/*/12FC/*m | | | |
| | A | | | |
| mAlb298-41 | mC*mU*mU*U*UAAUUmUmCmUmACU*G*U*U*d | 4313 | NT | NT |
| | GdTdAdGdAdT/i2FC//12FU//12FG//12FU | | | |
| | //12FA//12FA//12FC//12FG//12FA//12 | | | |
| | FU//12FC//12FG//12FG//12FG//12FA// | | | |
| | 12FA//12FC/*/12FU/*/12FG//12FG/*/i | | | |
| | 2FC/*mA | | | |
| mAlb298-42 | mC*mU*mU*U*UAAUUmUmCmUmACU*G*U*U*/ | 4314 | NT | NT |
| | 12FG//12FU//12FA//12FG//12FA//12FU | | | |
| | //12FC//12FU//12FG//12FU//12FA//12 | | | |
| | FA//12FC//12FG//12FA//12FU//12FC// | | | |
| | 12FG//12FG//12FG//12FA//12FA//12FC | | | |
| | /*/12FU/*/12FG//12FG/*/12FC/*mA | | | |
| mAlb298-43 | mC*mU*mU*U*UAAUUmUmCmUmAmCU*G*U*U* | 4315 | NT | NT |
| | GUAGAU/12FC//12FU//12FG//12FU//12F | | | |
| | A//12FA//12FC//12FG//12FA//12FU//i | | | |
| | 2FC//12FG//12FG//12FG//12FA//12FA/ | | | |
| | /12FC/*/12FU/*/12FG//12FG/*/12FC/* | | | |
| | mA | | | |

#: these guides had less than 75% purity based on analytical HPLC with purity ranging from 54 to 64%. All other guides exceeded 75% purity
NT: not tested
Nomenclature of chemical modifications: a “/” is used to separate bases with 2′-flourine modifications, m; 2′-O-methyl base (for example a A base with 2′-O-methyl modification is written as mA), i2F; internal 2′-flourine base (for example an internal C with 2′-flourine modification is written as /12FC/), 52F; 2′-flourine base at the 5′ end of the sequence (for example a 5′ C with 2′-flourine modification is written as /52FC/), 32F; 2′-flourine base at the 3′ end of the sequence (for example a 3′ A base with 2′-flourine modification is written as /32FA/), r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 12—Therapeutic Gene Editing in Mice Using Nucleases Described Herein

Gene editing platforms described herein have the potential to effect reparative alterations in vivo. Liver tissue is an example of a tissue that can be advantageously targeted using the gene editing compositions and systems described herein for in vivo gene editing, for example by introduction of indels that function to knock down expression of deleterious genes or that are used to replace defective genes. For example, several inherited diseases arise from defects in proteins expressed primarily in the liver, and in vivo delivery to the liver has been proven safe and effective in clinical trials of adeno-associated virus (AAV) vectors. Lipid nanoparticles have also been shown to deliver nucleic acids and approved drugs for RNAi strategies. Liver tissue also includes appropriate cellular machinery for efficient secretion of proteins into the systemic circulation.

Subjects having a condition in Table 13 or Table 14 are selected for gene editing therapy. For example, a human or mouse model subject having hemophilia A is identified for treatment with gene replacement therapy using a gene editing platform.

TABLE 6
Some Indications for Subject Selection in Therapeutic Gene Replacement

| Hemophilia A | Factor VIII | | 1 in 5,000 males |
|---|---|---|---|
| Hemophilia A | Factor VIII | Secreted | 1 in 5,000 males |
| Hemophilia B | Factor IX | Secreted | 1 in 20,000 |
| Hereditary | C1 inhibitor | Secreted | 1 in 25,000 |
| Angioedema | protein | | |
| Argininosuccinate | Argininosuccinate | Intracellular | 1 in 70,000 |
| Lyase deficiency | Lyase | | |
| Mucopolysaccharidosis | Arylsulfatase B | Intracellular | 1 in 200,000 |
| type IV (MPS IV), | | | |
| Hemophilia A | Factor VIII | | 1 in 5,000 males |
| Progressive familial | ATP binding | Intracellular | 1 in 50,000 |
| intrahepatic | cassette family B | | |
| cholestasis type 2 | | | |
| Classical galactosemia | Galactose-1- | Intracellular | 1 in 50,000 |
| | phosphate | | |
| | uridyltransferase | | |

TABLE 7
Some Indications for Subject Selection in Therapeutic Gene Knockdown

| Indication | Target Gene | Prevalence |
|---|---|---|
| Primary Hyperoxluria type I | Glyoxylate oxidase | Est 1 in 100,000, up to |
| | (HA01) | 5,000 patient in US + EU |
| Familial ATTR Amyloidoisis | Transthyretin | 1 in 100,000 in US, more |
| | | frequent in Japan, |
| | | Sweden |
| Acute Hepatic Porphoryia | Aminolevulinic | 1 in 50,000 |
| | Acid Synthase | |
| | (ALAS1) | |
| Cardiovascular disease without | PCSK9 | High (1 in 3 deaths in US |
| adequate LDL lowering by statins | | due to CVD) |
| Rare Hyperlipidemias | Angiopoietin like 3 | Various, approx 1 in |
| | | 500,000 |
| Homozygous Familial | ApoB100 | 1 in 1 million |
| Hypercholersterolemia | | |
| Hereditary Angioedema | Kallikrein | 1 in 25,000 |

A gene editing platform comprising a lipid nanoparticle (LNP) encapsulating an sgRNA and an mRNA encoding an MG nuclease described herein and an AAV (e.g., AAV serotype 8) comprising a donor template nucleic acid encoding a therapeutic gene are introduced into the liver intravenously to the subject. The LNP is targeted to hepatocytes via surface functionalization of the LNPs.

For example, the subject having hemophilia A is treated with a gene replacement platform comprising LNPs containing mRNA encoding a MG29-1 nuclease described herein (SEQ ID NO: 214). LNPs also contain sgRNA specific for albumin I, which is highly expressed in the liver (e.g., albumin can be expressed at about 5 g/dL in the liver, whereas factor VIII can be expressed at about 10 μg/dL in the liver, or 1 million times less than albumin). In addition to the LNPs, AAV8 (AAV serotype 8) viral particles comprising plasmids, which encode replacement template DNA encoding a replacement factor VIII nucleotide sequence, are delivered to the subject as well. Once inside the cell, the mRNA, sgRNA, and template DNA are transiently expressed. The MG29-1 nuclease targets the target locus of the host hepatocyte DNA using the sgRNA and then cleaves the host DNA. The donor template DNA transcribed from the plasmid delivered to the host hepatocyte in the AAV8 is spliced into the cell and stably integrated into the host DNA at the target site of the albumin I gene, and the inserted factor VIII DNA is expressed under the albumin promoter.

The gene editing platform is also used in subjects selected for gene knockdown therapy. For instance, a subject presenting with familial ATTR amyloidosis is treated with LNPs containing mRNA encoding an MG29-1 nuclease described herein (SEQ ID NO: 214) and a sgRNA specific to a target site in the transthyretin gene. The MG29-1 nuclease and sgRNA are delivered to and expressed in hepatocytes of the subject. In some embodiments, the sgRNA is targeted to a stop codon of the transthyretin gene, and the MG29-1 nuclease's activity removes the endogenous stop codon, effectively knocking down the expression of the gene. In some embodiments, the gene knockdown platform comprises an AAV8 containing a plasmid encoding a polynucleotide comprising a stop codon. When the AAV8 is delivered to the same cell that is expressing the nuclease and sgRNA, an exogenous stop codon is spliced into the tranthyretin gene, leading to knockdown of the gene's expression as a result of premature truncation of proteins translated from RNA produced from the edited DNA.

### Example 13—Gene Editing Outcomes at the DNA Level for CD38

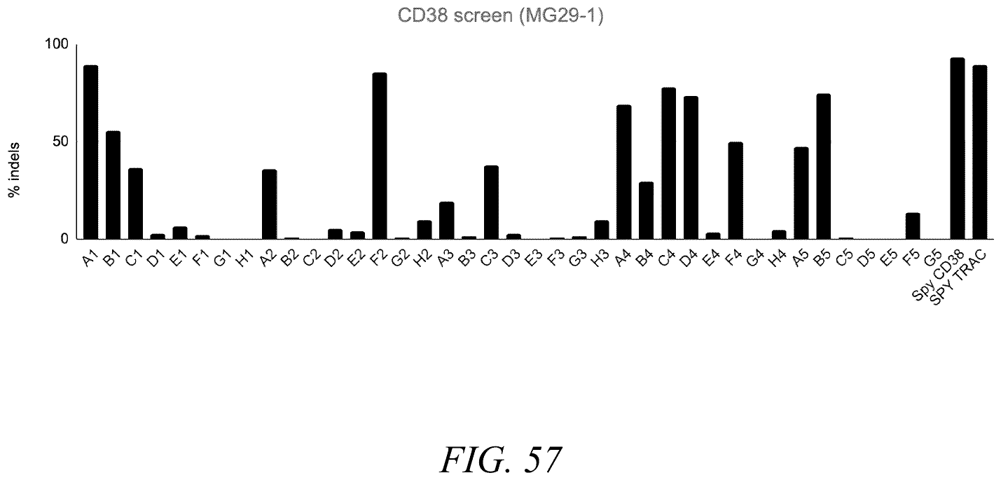

Primary NK cells were expanded using the NK Cloudz system (R&D Systems) according to the manufacturer's recommendations. Nucleofection of MG29-1 RNPs (212 pmol protein/320 pmol guide) (guide SEQ ID NOs: 4428-4465) was performed into NK cells (500,000) using the Lonza 4D electroporator. Cells were harvested and genomic DNA prepared five days post-transfection. PCR primers appropriate for use in NGS-based DNA sequencing were generated, optimized, and used to amplify the individual target sequences for each guide RNA (SEQ ID NOs: 4466-4503). The amplicons were sequenced on an Illumina MiSeq machine and analyzed with a proprietary Python script to measure gene editing (FIG. 57).

TABLE 14A
Sequences of Guide RNAs and Sequences Targeted Thereby for Example 37

| | SEQ ID | | |
|---|---|---|---|
| Guide Target | NO | Guide Name | SEQUENCE |
| MG29-1 | 4428 | MG29-1- | /AltR1/rUrArArUrUrUrCrUrArCrUrGrUrUrGrUrArGr |
| sgRNA | | CD38-sgRNA- | ArUrCrCrGrArGrArCrCrGrUrCrCrUrGrGrCrGrCrGr |
| targeting | | A1 | ArUrG/AltR2/ |
| CD38 | | | |
| MG29-1 | 4429 | MG29-1- | /AltR1/rUrArArUrUrUrCrUrArCrUrGrUrUrGrUrArGr |
| sgRNA | | CD38-sgRNA- | ArUrArGrUrGrUrArCrUrUrGrArCrGrCrArUrCrGrCr |
| targeting | | B1 | GrCrC/AltR2/ |
| CD38 | | | |
| MG29-1 | 4430 | MG29-1- | /AltR1/rUrArArUrUrUrCrUrArCrUrGrUrUrGrUrArGr |
| sgRNA | | CD38-sgRNA- | ArUrUrCrCrCrCrGrGrArCrArCrCrGrGrGrCrUrGrAr |
| targeting | | C1 | ArCrU/AltR2/ |
| CD38 | | | |
| MG29-1 | 4431 | MG29-1- | /AltR1/rUrArArUrUrUrCrUrArCrUrGrUrUrGrUrArGr |
| sgRNA | | CD38-sgRNA- | ArUrArArGrGrGrUrGrCrArUrUrUrArUrUrUrCrArAr |
| targeting | | D1 | ArArC/AltR2/ |
| CD38 | | | |
| MG29-1 | 4432 | MG29-1- | /AltR1/rUrArArUrUrUrCrUrArCrUrGrUrUrGrUrArGr |
| sgRNA | | CD38-sgRNA- | ArUrUrUrUrCrArArArArCrArUrCrCrUrUrGrCrArArC |
| targeting | | E1 | rArU/AltR2/ |
| CD38 | | | |
| MG29-1 | 4433 | MG29-1- | /AltR1/rUrArArUrUrUrCrUrArCrUrGrUrUrGrUrArGr |
| sgRNA | | CD38-sgRNA- | ArUrArArArArCrArUrCrCrUrUrGrCrArArCrArUrUrA |
| targeting | | F1 | rCrU/AltR2/ |
| CD38 | | | |
| MG29-1 | 4434 | MG29-1- | /AltR1/rUrArArUrUrUrCrUrArCrUrGrUrUrGrUrArGr |
| sgRNA | | CD38-sgRNA- | ArUrGrArArArUrArArArUrGrCrArCrCrCrUrUrGrAr |
| targeting | | G1 | ArArG/AltR2/ |
| CD38 | | | |
| MG29-1 | 4435 | MG29-1- | /AltR1/rUrArArUrUrUrCrUrArCrUrGrUrUrGrUrArGr |
| sgRNA | | CD38-sgRNA- | ArUrArArArUrArArArUrGrCrArCrCrCrUrUrGrArAr |
| targeting | | H1 | ArGrC/AltR2/ |
| CD38 | | | |
| MG29-1 | 4436 | MG29-1- | /AltR1/rUrArArUrUrUrCrUrArCrUrGrUrUrGrUrArGr |
| sgRNA | | CD38-sgRNA- | ArUrGrCrArGrUrCrUrArCrArUrGrUrCrUrGrArGrAr |
| targeting | | A2 | UrArA/AltR2/ |
| CD38 | | | |
| MG29-1 | 4437 | MG29-1- | /AltR1/rUrArArUrUrUrCrUrArCrUrGrUrUrGrUrArGr |
| sgRNA | | CD38-sgRNA- | ArUrGrArGrCrArGrArArUrArArArArGrArUrCrUrGr |
| targeting | | B2 | GrCrC/AltR2/ |
| CD38 | | | |
| MG29-1 | 4438 | MG29-1- | /AltR1/rUrArArUrUrUrCrUrArCrUrGrUrUrGrUrArGr |
| sgRNA | | CD38-sgRNA- | ArUrArUrUrCrUrGrCrUrCrCrArArArGrArArGrArAr |
| targeting | | C2 | UrCrU/AltR2/ |
| CD38 | | | |
| MG29-1 | 4439 | MG29-1- | /AltR1/rUrArArUrUrUrCrUrArCrUrGrUrUrGrUrArGr |
| sgRNA | | CD38-sgRNA- | ArUrUrUrCrUrGrCrUrCrCrArArArGrArArGrArArUr |
| targeting | | D2 | CrUrA/AltR2/ |
| CD38 | | | |
| MG29-1 | 4440 | MG29-1- | /AltR1/rUrArArUrUrUrCrUrArCrUrGrUrUrGrUrArGr |
| sgRNA | | CD38-sgRNA- | ArUrArGrUrArUrUrCrUrGrGrArArArArCrGrGrUrUr |
| targeting | | E2 | UrCrC/AltR2/ |
| CD38 | | | |
| MG29-1 | 4441 | MG29-1- | /AltR1/rUrArArUrUrUrCrUrArCrUrGrUrUrGrUrArGr |
| sgRNA | | CD38-sgRNA- | ArUrCrCrGrCrArGrGrGrUrArArGrUrArCrCrArArGr |
| targeting | | F2 | UrArG/AltR2/ |
| CD38 | | | |
| MG29-1 | 4442 | MG29-1- | /AltR1/rUrArArUrUrUrCrUrArCrUrGrUrUrGrUrArGr |
| sgRNA | | CD38-sgRNA- | ArUrCrCrArGrArArUrArCrUrGrArArArCrArGrGrGr |
| targeting | | G2 | UrUrG/AltR2/ |
| CD38 | | | |
| MG29-1 | 4443 | MG29-1- | /AltR1/rUrArArUrUrUrCrUrArCrUrGrUrUrGrUrArGr |
| sgRNA | | CD38-sgRNA- | ArUrCrArGrArArUrArCrUrGrArArArCrArGrGrGrUr |
| targeting | | H2 | UrGrU/AltR2/ |
| CD38 | | | |
| MG29-1 | 4444 | MG29-1- | /AltR1/rUrArArUrUrUrCrUrArCrUrGrUrUrGrUrArGr |
| sgRNA | | CD38-sgRNA- | ArUrUrCrCrArGrUrCrUrGrGrGrCrArArGrArUrUrGr |
| targeting | | A3 | ArUrA/AltR2/ |
| CD38 | | | |
| MG29-1 | 4445 | MG29-1- | /AltR1/rUrArArUrUrUrCrUrArCrUrGrUrUrGrUrArGr |
| sgRNA | | CD38-sgRNA- | ArUrUrUrUrCrUrArArArArGrArCrArUrArGrUrUrUr |
| targeting | | B3 | GrUrA/AltR2/ |
| CD38 | | | |
| MG29-1 | 4446 | MG29-1- | /AltR1/rUrArArUrUrUrCrUrArCrUrGrUrUrGrUrArGr |
| sgRNA | | CD38-sgRNA- | ArUrCrArGrArArGrCrUrGrCrCrUrGrUrGrArUrGrUr |
| targeting | | C3 | GrGrU/AltR2/ |
| CD38 | | | |
| MG29-1 | 4447 | MG29-1- | /AltR1/rUrArArUrUrUrCrUrArCrUrGrUrUrGrUrArGr |
| sgRNA | | CD38-sgRNA- | ArUrArCrArArArArArCrArGrGrUrArCrArCrArUrUr |
| targeting | | D3 | UrArU/AltR2/ |
| CD38 | | | |
| MG29-1 | 4448 | MG29-1- | /AltR1/rUrArArUrUrUrCrUrArCrUrGrUrUrGrUrArGr |
| sgRNA | | CD38-sgRNA- | ArUrUrGrUrCrArArArGrArUrUrUrUrArCrUrGrCrGr |
| targeting | | E3 | GrGrA/AltR2/ |
| CD38 | | | |
| MG29-1 | 4449 | MG29-1- | /AltR1/rUrArArUrUrUrCrUrArCrUrGrUrUrGrUrArGr |
| sgRNA | | CD38-sgRNA- | ArUrGrUrCrArArArGrArUrUrUrUrArCrUrGrCrGrGr |
| targeting | | F3 | GrArU/AltR2/ |
| CD38 | | | |
| MG29-1 | 4450 | MG29-1- | /AltR1/rUrArArUrUrUrCrUrArCrUrGrUrUrGrUrArGr |
| sgRNA | | CD38-sgRNA- | ArUrUrCrArArArGrArUrUrUrUrArCrUrGrCrGrGrGr |
| targeting | | G3 | ArUrC/AltR2/ |
| CD38 | | | |
| MG29-1 | 4451 | MG29-1- | /AltR1/rUrArArUrUrUrCrUrArCrUrGrUrUrGrUrArGr |
| sgRNA | | CD38-sgRNA- | ArUrArCrUrGrCrGrGrGrArUrCrCrArUrUrGrArGrCr |
| targeting | | H3 | ArUrC/AltR2/ |
| CD38 | | | |
| MG29-1 | 4452 | MG29-1- | /AltR1/rUrArArUrUrUrCrUrArCrUrGrUrUrGrUrArGr |
| sgRNA | | CD38-sgRNA- | ArUrCrUrGrCrGrGrGrArUrCrCrArUrUrGrArGrCrAr |
| targeting | | A4 | UrCrA/AltR2/ |
| CD38 | | | |
| MG29-1 | 4453 | MG29-1- | /AltR1/rUrArArUrUrUrCrUrArCrUrGrUrUrGrUrArGr |
| sgRNA | | CD38-sgRNA- | ArUrGrGrGrArGrUrGrUrGrGrArArGrUrCrCrArUrAr |
| targeting | | B4 | ArUrU/AltR2/ |
| CD38 | | | |
| MG29-1 | 4454 | MG29-1- | /AltR1/rUrArArUrUrUrCrUrArCrUrGrUrUrGrUrArGr |
| sgRNA | | CD38-sgRNA- | ArUrGrGrArGrUrGrUrGrGrArArGrUrCrCrArUrArAr |
| targeting | | C4 | UrUrU/AltR2/ |
| CD38 | | | |
| MG29-1 | 4455 | MG29-1- | /AltR1/rUrArArUrUrUrCrUrArCrUrGrUrUrGrUrArGr |
| sgRNA | | CD38-sgRNA- | ArUrCrArArCrCrArGrArGrArArGrGrUrUrCrArGrAr |
| targeting | | D4 | CrArC/AltR2/ |
| CD38 | | | |
| MG29-1 | 4456 | MG29-1- | /AltR1/rUrArArUrUrUrCrUrArCrUrGrUrUrGrUrArGr |
| sgRNA | | CD38-sgRNA- | ArUrCrCrUrGrCrArArGrArArUrArUrCrUrArCrArGr |
| targeting | | E4 | GrUrA/AltR2/ |
| CD38 | | | |
| MG29-1 | 4457 | MG29-1- | /AltR1/rUrArArUrUrUrCrUrArCrUrGrUrUrGrUrArGr |
| sgRNA | | CD38-sgRNA- | ArUrCrUrGrCrArArGrArArUrArUrCrUrArCrArGrGr |
| targeting | | F4 | UrArA/AltR2/ |
| CD38 | | | |
| MG29-1 | 4458 | MG29-1- | /AltR1/rUrArArUrUrUrCrUrArCrUrGrUrUrGrUrArGr |
| sgRNA | | CD38-sgRNA- | ArUrGrCrUrUrArUrArArUrCrGrArUrUrCrCrArGrCr |
| targeting | | G4 | UrCrU/AltR2/ |
| CD38 | | | |
| MG29-1 | 4459 | MG29-1- | /AltR1/rUrArArUrUrUrCrUrArCrUrGrUrUrGrUrArGr |
| sgRNA | | CD38-sgRNA- | ArUrCrUrUrArUrArArUrCrGrArUrUrCrCrArGrCrUr |
| targeting | | H4 | CrUrU/AltR2/ |
| CD38 | | | |
| MG29-1 | 4460 | MG29-1- | /AltR1/rUrArArUrUrUrCrUrArCrUrGrUrUrGrUrArGr |
| sgRNA | | CD38-sgRNA- | ArUrArUrGrGrUrGrGrGrArUrCrCrUrGrGrCrArUrAr |
| targeting | | A5 | ArGrU/AltR2/ |
| CD38 | | | |
| MG29-1 | 4461 | MG29-1- | /AltR1/rUrArArUrUrUrCrUrArCrUrGrUrUrGrUrArGr |
| sgRNA | | CD38-sgRNA- | ArUrUrGrGrUrGrGrGrArUrCrCrUrGrGrCrArUrArAr |
| targeting | | B5 | GrUrC/AltR2/ |
| CD38 | | | |
| MG29-1 | 4462 | MG29-1- | /AltR1/rUrArArUrUrUrCrUrArCrUrGrUrUrGrUrArGr |
| sgRNA | | CD38-sgRNA- | ArUrUrUrCrArGrUrGrUrGrUrGrArArArArArUrCrCr |
| targeting | | C5 | UrGrA/AltR2/ |
| CD38 | | | |
| MG29-1 | 4463 | MG29-1- | /AltR1/rUrArArUrUrUrCrUrArCrUrGrUrUrGrUrArGr |
| gRNA | | CD38-sgRNA- | ArUrUrCrArCrArCrArCrUrGrArArGrArArArCrUrUr |
| targeting | | D5 | GrUrC/AltR2/ |
| CD38 | | | |
| MG29-1 | 4464 | MG29-1- | /AltR1/rUrArArUrUrUrCrUrArCrUrGrUrUrGrUrArGr |
| sgRNA | | CD38-sgRNA- | ArUrCrArCrArCrArCrUrGrArArGrArArArCrUrUrGr |
| targeting | | E5 | UrCrA/AltR2/ |
| CD38 | | | |
| MG29-1 | 4465 | MG29-1- | /AltR1/rUrArArUrUrUrCrUrArCrUrGrUrUrGrUrArGr |
| sgRNA | | CD38-sgRNA- | ArUrArCrArCrArCrUrGrArArGrArArArCrUrUrGrUr |
| targeting | | F5 | CrArG/AltR2/ |
| CD38 | | | |
| DNA sequence | 4466 | MG29-1- | CCGAGACCGTCCTGGCGCGATG |
| of CD38 target | | CD38-target | |
| site | | site-A1 | |
| DNA sequence | 4467 | MG29-1- | AGTGTACTTGACGCATCGCGCC |
| of CD38 target | | CD38-target | |
| site | | site-B1 | |
| DNA sequence | 4468 | MG29-1- | TCCCCGGACACCGGGCTGAACT |
| of CD38 target | | CD38-target | |
| site | | site-C1 | |
| DNA sequence | 4469 | MG29-1- | AAGGGTGCATTTATTTCAAAAC |
| of CD38 target | | CD38-target | |
| site | | site-D1 | |
| DNA sequence | 4470 | MG29-1- | TTTCAAAACATCCTTGCAACAT |
| of CD38 target | | CD38-target | |
| site | | site-E1 | |
| DNA sequence | 4471 | MG29-1- | AAAACATCCTTGCAACATTACT |
| of CD38 target | | CD38-target | |
| site | | site-F1 | |
| DNA sequence | 4472 | MG29-1- | GAAATAAATGCACCCTTGAAAG |
| of CD38 target | | CD38-target | |
| site | | site-G1 | |
| DNA sequence | 4473 | MG29-1- | AAATAAATGCACCCTTGAAAGC |
| of CD38 target | | CD38-target | |
| site | | site-H1 | |
| DNA sequence | 4474 | MG29-1- | GCAGTCTACATGTCTGAGATAA |
| of CD38 target | | CD38-target | |
| site | | site-A2 | |
| DNA sequence | 4475 | MG29-1- | GAGCAGAATAAAAGATCTGGCC |
| of CD38 target | | CD38-target | |
| site | | site-B2 | |
| DNA sequence | 4476 | MG29-1- | ATTCTGCTCCAAAGAAGAATCT |
| of CD38 target | | CD38-target | |
| site | | site-C2 | |
| DNA sequence | 4477 | MG29-1- | TTCTGCTCCAAAGAAGAATCTA |
| of CD38 target | | CD38-target | |
| site | | site-D2 | |
| DNA sequence | 4478 | MG29-1- | AGTATTCTGGAAAACGGTTTCC |
| of CD38 target | | CD38-target | |
| site | | site-E2 | |
| DNA sequence | 4479 | MG29-1- | CCGCAGGGTAAGTACCAAGTAG |
| of CD38 target | | CD38-target | |
| site | | site-F2 | |
| DNA sequence | 4480 | MG29-1- | CCAGAATACTGAAACAGGGTTG |
| of CD38 target | | CD38-target | |
| site | | site-G2 | |
| DNA sequence | 4481 | MG29-1- | CAGAATACTGAAACAGGGTTGT |
| of CD38 target | | CD38-target | |
| site | | site-H2 | |
| DNA sequence | 4482 | MG29-1- | TCCAGTCTGGGCAAGATTGATA |
| of CD38 target | | CD38-target | |
| site | | site-A3 | |
| DNA sequence | 4483 | MG29-1- | TTTCTAAAAGACATAGTTTGTA |
| of CD38 target | | CD38-target | |
| site | | site-B3 | |
| DNA sequence | 4484 | MG29-1- | CAGAAGCTGCCTGTGATGTGGT |
| of CD38 target | | CD38-target | |
| site | | site-C3 | |
| DNA sequence | 4485 | MG29-1- | ACAAAAACAGGTACACATTTAT |
| of CD38 target | | CD38-target | |
| site | | site-D3 | |
| DNA sequence | 4486 | MG29-1- | TGTCAAAGATTTTACTGCGGGA |
| of CD38 target | | CD38-target | |
| site | | site-E3 | |
| DNA sequence | 4487 | MG29-1- | GTCAAAGATTTTACTGCGGGAT |
| of CD38 target | | CD38-target | |
| site | | site-F3 | |
| DNA sequence | 4488 | MG29-1- | TCAAAGATTTTACTGCGGGATC |
| of CD38 target | | CD38-target | |
| site | | site-G3 | |
| DNA sequence | 4489 | MG29-1- | ACTGCGGGATCCATTGAGCATC |
| of CD38 target | | CD38-target | |
| site | | site-H3 | |
| DNA sequence | 4490 | MG29-1- | CTGCGGGATCCATTGAGCATCA |
| of CD38 target | | CD38-target | |
| site | | site-A4 | |
| DNA sequence | 4491 | MG29-1- | GGGAGTGTGGAAGTCCATAATT |
| of CD38 target | | CD38-target | |
| site | | site-B4 | |
| DNA sequence | 4492 | MG29-1- | GGAGTGTGGAAGTCCATAATTT |
| of CD38 target | | CD38-target | |
| site | | site-C4 | |
| DNA sequence | 4493 | MG29-1- | CAACCAGAGAAGGTTCAGACAC |
| of CD38 target | | CD38-target | |
| site | | site-D4 | |
| DNA sequence | 4494 | MG29-1- | CCTGCAAGAATATCTACAGGTA |
| of CD38 target | | CD38-target | |
| site | | site-E4 | |
| DNA sequence | 4495 | MG29-1- | CTGCAAGAATATCTACAGGTAA |
| of CD38 target | | CD38-target | |
| site | | site-F4 | |
| DNA sequence | 4496 | MG29-1- | GCTTATAATCGATTCCAGCTCT |
| of CD38 target | | CD38-target | |
| site | | site-G4 | |
| DNA sequence | 4497 | MG29-1- | CTTATAATCGATTCCAGCTCTT |
| of CD38 target | | CD38-target | |
| site | | site-H4 | |
| DNA sequence | 4498 | MG29-1- | ATGGTGGGATCCTGGCATAAGT |
| of CD38 target | | CD38-target | |
| site | | site-A5 | |
| DNA sequence | 4499 | MG29-1- | TGGTGGGATCCTGGCATAAGTC |
| of CD38 target | | CD38-target | |
| site | | site-B5 | |
| DNA sequence | 4500 | MG29-1- | TTCAGTGTGTGAAAAATCCTGA |
| of CD38 target | | CD38-target | |
| site | | site-C5 | |
| DNA sequence | 4501 | MG29-1- | TCACACACTGAAGAAACTTGTC |
| of CD38 target | | CD38-target | |
| site | | site-D5 | |
| DNA sequence | 4502 | MG29-1- | CACACACTGAAGAAACTTGTCA |
| of CD38 target | | CD38-target | |
| site | | site-E5 | |
| DNA sequence | 4503 | MG29-1- | ACACACTGAAGAAACTTGTCAG |
| of CD38 target | | CD38-target | |
| site | | site-F5 | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 38—Gene Editing Outcomes at the Phenotypic Level for CD38

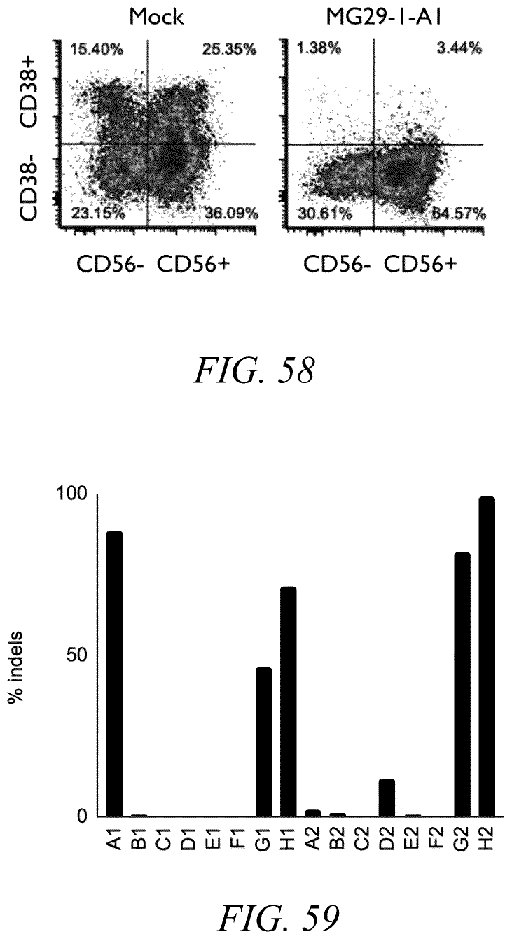

Edited cells were assayed for CD38 expression as follows: primary NK cells (500,000) were washed with phosphate buffered saline (PBS) and stained with LIVE/DEAD™ Fixable Aqua Dead Cell Stain Kit (ThermoFisher) according to the manufacturer specifications. Cells were then washed and incubated with FC Block—Human TruStain FcX™ (Biolegend) for 20 minutes on ice. Next, cells were stained with anti-CD56 PE and anti-38 PerCP eFluor 710 antibodies (ThermoFisher) according to the manufacturer recommendations and analyzed by flow cytometry by collecting 25,000 total events per specimen (FIG. 58).

### Example 39—Gene Editing Outcomes at the DNA Level for TIGIT

Primary T cells were purified from PMBCs using a negative selection kit (Miltenyi) according to the manufacturer's recommendations. Nucleofection of MG29-1 RNPs (106 pmol protein/160 pmol guide) (SEQ ID NOs: 4504-4520) was performed into T cells (200,000) using the Lonza 4D electroporator. Cells were harvested and genomic DNA prepared five days post-transfection. PCR primers appropriate for use in NGS-based DNA sequencing were generated, optimized, and used to amplify the individual target sequences for each guide RNA (SEQ ID NOs: 4521-4537). The amplicons were sequenced on an Illumina MiSeq machine and analyzed with a proprietary Python script to measure gene editing (FIG. 59).

TABLE 14B
Sequences of Guide RNAs and Sequences Targeted for Example 39

| | SEQ | | |
|---|---|---|---|
| Guide | ID | | |
| Target | NO | Guide Name | SEQUENCE |
| MG29-1 | 4504 | MG29-1-TIGIT- | /AltR1/rUrArArUrUrUrCrUrArCrUrGrUrUrGrUrArGrArUrArG |
| sgRNA | | sgRNA-A1 | rGrCrCrUrUrArCrCrUrGrArGrGrCrGrArGrGrGrG/AltR2/ |
| targeting | | | |
| TIGIT | | | |
| MG29-1 | 4505 | MG29-1-TIGIT- | /AltR1/rUrArArUrUrUrCrUrArCrUrGrUrUrGrUrArGrArUrUrA |
| sgRNA | | sgRNA-B1 | rUrUrGrUrGrCrCrUrGrUrCrArUrCrArUrUrCrCrU/AltR2/ |
| targeting | | | |
| TIGIT | | | |
| MG29-1 | 4506 | MG29-1-TIGIT- | /AltR1/rUrArArUrUrUrCrUrArCrUrGrUrUrGrUrArGrArUrUrCr |
| sgRNA | | sgRNA-C1 | UrGrCrArGrArArArUrGrUrUrCrCrCrCrGrUrUrG/AltR2/ |
| targeting | | | |
| TIGIT | | | |
| MG29-1 | 4507 | MG29-1-TIGIT- | /AltR1/rUrArArUrUrUrCrUrArCrUrGrUrUrGrUrArGrArUrUrG |
| sgRNA | | sgRNA-D1 | rCrArGrArGrArArArGrGrUrGrGrCrUrCrUrArUrC/AltR2/ |
| targeting | | | |
| TIGIT | | | |
| MG29-1 | 4508 | MG29-1-TIGIT- | /AltR1/rUrArArUrUrUrCrUrArCrUrGrUrUrGrUrArGrArUrUrA |
| sgRNA | | sgRNA-E1 | rArUrGrCrUrGrArCrUrUrGrGrGrGrUrGrGrCrArC/AltR2/ |
| targeting | | | |
| TIGIT | | | |
| MG29-1 | 4509 | MG29-1-TIGIT- | /AltR1/rUrArArUrUrUrCrUrArCrUrGrUrUrGrUrArGrArUrUrA |
| sgRNA | | sgRNA-F1 | rGrGrArCrCrUrCrCrArGrGrArArGrArUrUrCrUrC/AltR2/ |
| targeting | | | |
| TIGIT | | | |
| MG29-1 | 4510 | MG29-1-TIGIT- | /AltR1/rUrArArUrUrUrCrUrArCrUrGrUrUrGrUrArGrArUrGrU |
| sgRNA | | sgRNA-G1 | rCrCrUrCrCrCrUrCrUrArGrUrGrGrCrUrGrArGrC/AltR2/ |
| targeting | | | |
| TIGIT | | | |
| MG29-1 | 4511 | MG29-1-TIGIT- | /AltR1/rUrArArUrUrUrCrUrArCrUrGrUrUrGrUrArGrArUrUrCr |
| sgRNA | | sgRNA-H1 | CrUrCrCrCrUrCrUrArGrUrGrGrCrUrGrArGrCrA/AltR2/ |
| targeting | | | |
| TIGIT | | | |
| MG29-1 | 4512 | MG29-1-TIGIT- | /AltR1/rUrArArUrUrUrCrUrArCrUrGrUrUrGrUrArGrArUrUrA |
| sgRNA | | sgRNA-A2 | rGrUrCrArArCrGrCrGrArCrCrArCrCrArCrGrArU/AltR2/ |
| targeting | | | |
| TIGIT | | | |
| MG29-1 | 4513 | MG29-1-TIGIT- | /AltR1/rUrArArUrUrUrCrUrArCrUrGrUrUrGrUrArGrArUrUrA |
| sgRNA | | sgRNA-B2 | rGrUrUrUrGrUrUrUrGrUrUrUrUrUrArGrArArGrA/AltR2/ |
| targeting | | | |
| TIGIT | | | |
| MG29-1 | 4514 | MG29-1-TIGIT- | /AltR1/rUrArArUrUrUrCrUrArCrUrGrUrUrGrUrArGrArUrUrU |
| sgRNA | | sgRNA-C2 | rUrGrUrUrUrUrUrArGrArArGrArArArGrCrCrCrU/AltR2/ |
| targeting | | | |
| TIGIT | | | |
| MG29-1 | 4515 | MG29-1-TIGIT- | /AltR1/rUrArArUrUrUrCrUrArCrUrGrUrUrGrUrArGrArUrUrU |
| sgRNA | | sgRNA-D2 | rUrUrUrArGrArArGrArArArGrCrCrCrUrCrArGrA/AltR2/ |
| targeting | | | |
| TIGIT | | | |
| MG29-1 | 4516 | MG29-1-TIGIT- | /AltR1/rUrArArUrUrUrCrUrArCrUrGrUrUrGrUrArGrArUrUrA |
| sgRNA | | sgRNA-E2 | rGrArArGrArArArGrCrCrCrUrCrArGrArArUrCrC/AltR2/ |
| targeting | | | |
| TIGIT | | | |
| MG29-1 | 4517 | MG29-1-TIGIT- | /AltR1/rUrArArUrUrUrCrUrArCrUrGrUrUrGrUrArGrArUrArG |
| sgRNA | | sgRNA-F2 | rArArGrArArArGrCrCrCrUrCrArGrArArUrCrCrA/AltR2/ |
| targeting | | | |
| TIGIT | | | |
| MG29-1 | 4518 | MG29-1-TIGIT- | /AltR1/rUrArArUrUrUrCrUrArCrUrGrUrUrGrUrArGrArUrGrA |
| sgRNA | | sgRNA-G2 | rArGrArArArGrCrCrCrUrCrArGrArArUrCrCrArU/AltR2/ |
| targeting | | | |
| TIGIT | | | |
| MG29-1 | 4519 | MG29-1-TIGIT- | /AltR1/rUrArArUrUrUrCrUrArCrUrGrUrUrGrUrArGrArUrUrCr |
| sgRNA | | sgRNA-H2 | CrUrGrArGrGrUrCrArCrCrUrUrCrCrArCrArGrA/AltR2/ |
| targeting | | | |
| TIGIT | | | |
| MG29-1 | 4520 | MG29-1-TIGIT- | /AltR1/rUrArArUrUrUrCrUrArCrUrGrUrUrGrUrArGrArUrCrUr |
| sgRNA | | sgRNA-A3 | CrCrUrGrArGrGrUrCrArCrCrUrUrCrCrArCrArG/AltR2/ |
| targeting | | | |
| TIGIT | | | |
| DNA | 4521 | MG29-1-TIGIT- | AGGCCTTACCTGAGGCGAGGGG |
| sequence of | | target site-A1 | |
| TIGIT target | | | |
| site | | | |
| DNA | 4522 | MG29-1-TIGIT- | TATTGTGCCTGTCATCATTCCT |
| sequence of | | target site-B1 | |
| TIGIT target | | | |
| site | | | |
| DNA | 4523 | MG29-1-TIGIT- | TCTGCAGAAATGTTCCCCGTTG |
| sequence of | | target site-C1 | |
| TIGIT target | | | |
| site | | | |
| DNA | 4524 | MG29-1-TIGIT- | TGCAGAGAAAGGTGGCTCTATC |
| sequence of | | target site-D1 | |
| TIGIT target | | | |
| site | | | |
| DNA | 4525 | MG29-1-TIGIT- | TAATGCTGACTTGGGGTGGCAC |
| sequence of | | target site-E1 | |
| TIGIT target | | | |
| site | | | |
| DNA | 4526 | MG29-1-TIGIT- | TAGGACCTCCAGGAAGATTCTC |
| sequence of | | target site-F1 | |
| TIGIT target | | | |
| site | | | |
| DNA | 4527 | MG29-1-TIGIT- | GTCCTCCCTCTAGTGGCTGAGC |
| sequence of | | target site-G1 | |
| TIGIT target | | | |
| site | | | |
| DNA | 4528 | MG29-1-TIGIT- | TCCTCCCTCTAGTGGCTGAGCA |
| sequence of | | target site-H1 | |
| TIGIT target | | | |
| site | | | |
| DNA | 4529 | MG29-1-TIGIT- | TAGTCAACGCGACCACCACGAT |
| sequence of | | target site-A2 | |
| TIGIT target | | | |
| site | | | |
| DNA | 4530 | MG29-1-TIGIT- | TAGTTTGTTTGTTTTTAGAAGA |
| sequence of | | target site-B2 | |
| TIGIT target | | | |
| site | | | |
| DNA | 4531 | MG29-1-TIGIT- | TTTGTTTTTAGAAGAAAGCCCT |
| sequence of | | target site-C2 | |
| TIGIT target | | | |
| site | | | |
| DNA | 4532 | MG29-1-TIGIT- | TTTTTAGAAGAAAGCCCTCAGA |
| sequence of | | target site-D2 | |
| TIGIT target | | | |
| site | | | |
| DNA | 4533 | MG29-1-TIGIT- | TAGAAGAAAGCCCTCAGAATCC |
| sequence of | | target site-E2 | |
| TIGIT target | | | |
| site | | | |
| DNA | 4534 | MG29-1-TIGIT- | AGAAGAAAGCCCTCAGAATCCA |
| sequence of | | target site-F2 | |
| TIGIT target | | | |
| site | | | |
| DNA | 4535 | MG29-1-TIGIT- | GAAGAAAGCCCTCAGAATCCAT |
| sequence of | | target site-G2 | |
| TIGIT target | | | |
| site | | | |
| DNA | 4536 | MG29-1-TIGIT- | CTCCTGAGGTCACCTTCCACAG |
| sequence of | | target site-H2 | |
| TIGIT target | | | |
| site | | | |
| DNA | 4537 | MG29-1-TIGIT- | TCCTGAGGTCACCTTCCACAGA |
| sequence of | | target site-A3 | |
| TIGIT target | | | |
| site | | | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 14—Gene Editing Outcomes at the DNA Level for AAVS1

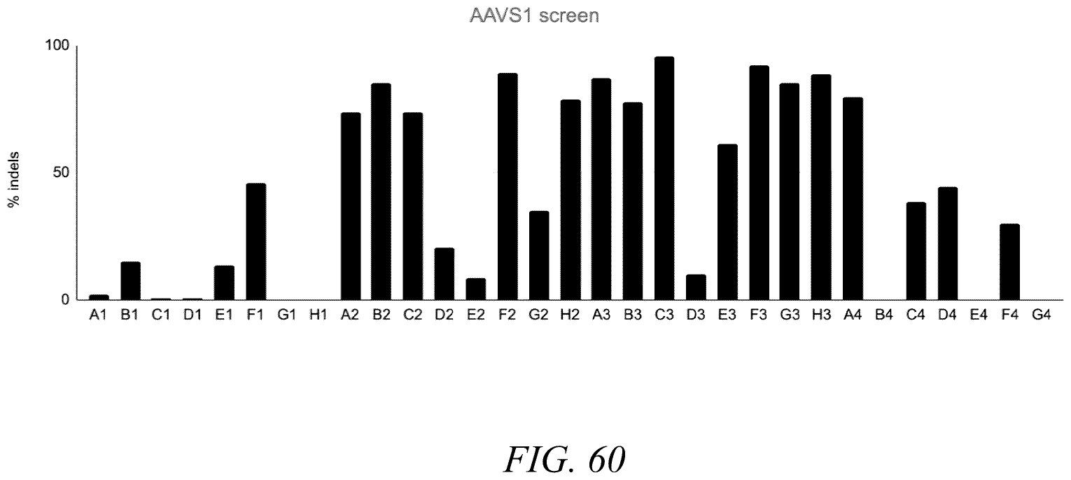

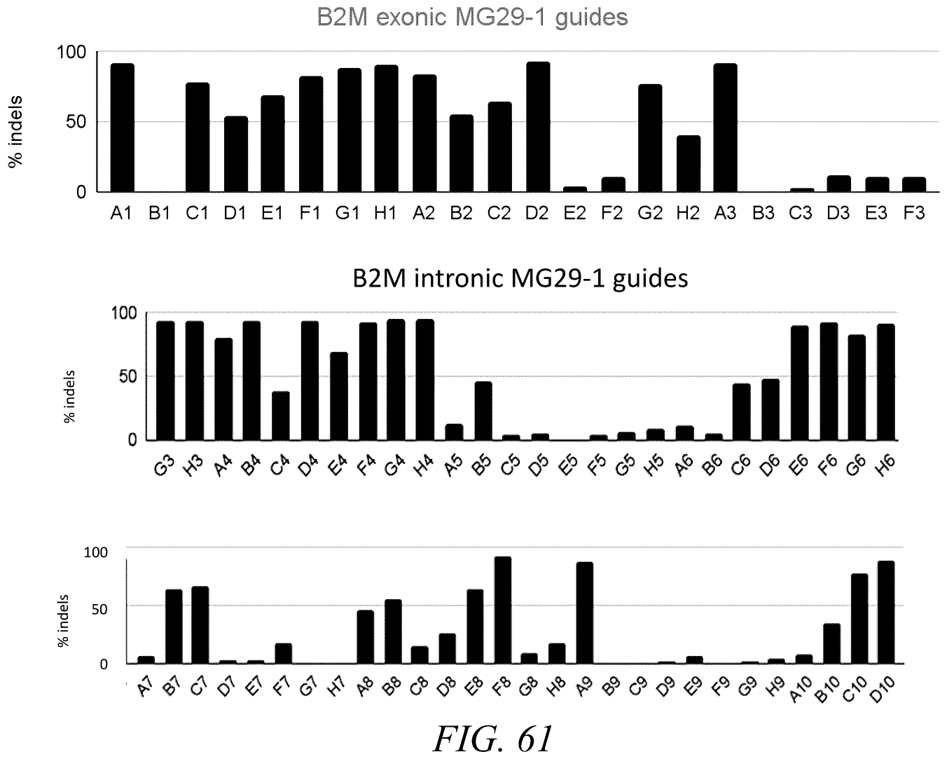

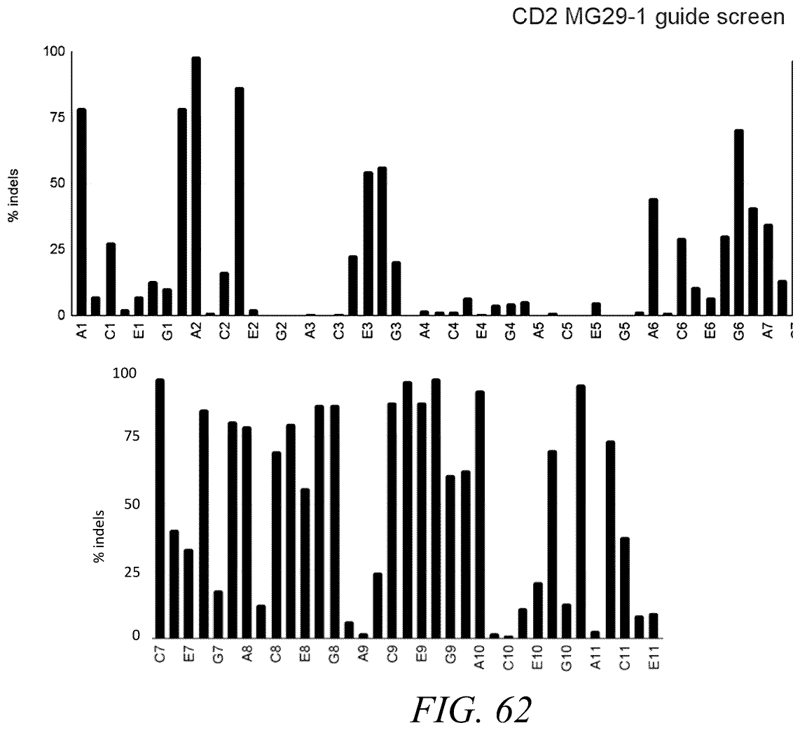

Primary T cells were purified from PMBCs using a negative selection kit (Miltenyi) according to the manufacturer's recommendations. Nucleofection of MG29-1 RNPs (106 pmol protein/160 pmol guide) (SEQ ID NOs: 4538-4568) was performed into T cells (200,000) using the Lonza 4D electroporator. Cells were harvested and genomic DNA prepared five days post-transfection. PCR primers appropriate for use in NGS-based DNA sequencing were generated, optimized, and used to amplify the individual target sequences for each guide RNA (SEQ ID NOs: 4569-4599). The amplicons were sequenced on an Illumina MiSeq machine and analyzed with a proprietary Python script to measure gene editing (FIG. 60).

TABLE 14C
Sequences of Guide RNAs and Sequences Targeted for Example 40

| | SEQ | | |
|---|---|---|---|
| | ID | | |
| Guide Target | NO | Guide Name | SEQUENCE |
| MG29-1 | 4538 | MG29-1-AAVS1- | /AltR1/rUrArArUrUrUrCrUrArCrUrGrUrUrGrUrArGrA |
| sgRNA | | sgRNA-A1 | rUrGrUrUrUrUrUrCrCrArArArCrUrGrCrUrUrCrUrCr |
| targeting | | | CrU/AltR2/ |
| AAVS1 | | | |
| MG29-1 | 4539 | MG29-1-AAVS1- | /AltR1/rUrArArUrUrUrCrUrArCrUrGrUrUrGrUrArGrA |
| sgRNA | | sgRNA-B1 | rUrUrUrUrUrUrCrCrArArArCrUrGrCrUrUrCrUrCrCr |
| targeting | | | UrC/AltR2/ |
| AAVS1 | | | |
| MG29-1 | 4540 | MG29-1-AAVS1- | /AltR1/rUrArArUrUrUrCrUrArCrUrGrUrUrGrUrArGrA |
| sgRNA | | sgRNA-C1 | rUrUrCrCrArArArCrUrGrCrUrUrCrUrCrCrUrCrUrUr |
| targeting | | | GrG/AltR2/ |
| AAVS1 | | | |
| MG29-1 | 4541 | MG29-1-AAVS1- | /AltR1/rUrArArUrUrUrCrUrArCrUrGrUrUrGrUrArGrA |
| sgRNA | | sgRNA-D1 | rUrCrCrArArArCrUrGrCrUrUrCrUrCrCrUrCrUrUrGr |
| targeting | | | GrG/AltR2/ |
| AAVS1 | | | |
| MG29-1 | 4542 | MG29-1-AAVS1- | /AltR1/rUrArArUrUrUrCrUrArCrUrGrUrUrGrUrArGrA |
| sgRNA | | sgRNA-E1 | rUrCrArArArCrUrGrCrUrUrCrUrCrCrUrCrUrUrGrGr |
| targeting | | | GrA/AltR2/ |
| AAVS1 | | | |
| MG29-1 | 4543 | MG29-1-AAVS1- | /AltR1/rUrArArUrUrUrCrUrArCrUrGrUrUrGrUrArGrA |
| sgRNA | | sgRNA-F1 | rUrCrUrGrUrCrArCrCrArArUrCrCrUrGrUrCrCrCrUr |
| targeting | | | ArG/AltR2/ |
| AAVS1 | | | |
| MG29-1 | 4544 | MG29-1-AAVS1- | /AltR1/rUrArArUrUrUrCrUrArCrUrGrUrUrGrUrArGrA |
| sgRNA | | sgRNA-G1 | rUrUrGrUrCrArCrCrArArUrCrCrUrGrUrCrCrCrUrAr |
| targeting | | | GrU/AltR2/ |
| AAVS1 | | | |
| MG29-1 | 4545 | MG29-1-AAVS1- | /AltR1/rUrArArUrUrUrCrUrArCrUrGrUrUrGrUrArGrA |
| sgRNA | | sgRNA-H1 | rUrGrGrGrUrUrGrUrCrCrArGrArArArArArCrGrGrUr |
| targeting | | | GrA/AltR2/ |
| AAVS1 | | | |
| MG29-1 | 4546 | MG29-1-AAVS1- | /AltR1/rUrArArUrUrUrCrUrArCrUrGrUrUrGrUrArGrA |
| sgRNA | | sgRNA-A2 | rUrCrCrUrUrCrUrCrCrUrUrCrUrGrGrGrGrCrCrUrGr |
| targeting | | | UrG/AltR2/ |
| AAVS1 | | | |
| MG29-1 | 4547 | MG29-1-AAVS1- | /AltR1/rUrArArUrUrUrCrUrArCrUrGrUrUrGrUrArGrA |
| sgRNA | | sgRNA-B2 | rUrCrUrUrCrUrCrCrUrUrCrUrGrGrGrGrCrCrUrGrUr |
| targeting | | | GrC/AltR2/ |
| AAVS1 | | | |
| MG29-1 | 4548 | MG29-1-AAVS1- | /AltR1/rUrArArUrUrUrCrUrArCrUrGrUrUrGrUrArGrA |
| sgRNA | | sgRNA-C2 | rUrUrUrArGrGrArUrGrGrCrCrUrUrCrUrCrCrGrArCr |
| targeting | | | GrG/AltR2/ |
| AAVS1 | | | |
| MG29-1 | 4549 | MG29-1-AAVS1- | /AltR1/rUrArArUrUrUrCrUrArCrUrGrUrUrGrUrArGrA |
| sgRNA | | sgRNA-D2 | rUrUrCrUrGrGrArCrArArCrCrCrCrArArArGrUrArCr |
| targeting | | | CrC/AltR2/ |
| AAVS1 | | | |
| MG29-1 | 4550 | MG29-1-AAVS1- | /AltR1/rUrArArUrUrUrCrUrArCrUrGrUrUrGrUrArGrA |
| sgRNA | | sgRNA-E2 | rUrCrUrGrGrArCrArArCrCrCrCrArArArGrUrArCrCr |
| targeting | | | CrC/AltR2/ |
| AAVS1 | | | |
| MG29-1 | 4551 | MG29-1-AAVS1- | /AltR1/rUrArArUrUrUrCrUrArCrUrGrUrUrGrUrArGrA |
| sgRNA | | sgRNA-F2 | rUrUrGrGrArCrArArCrCrCrCrArArArGrUrArCrCrCr |
| targeting | | | CrG/AltR2/ |
| AAVS1 | | | |
| MG29-1 | 4552 | MG29-1-AAVS1- | /AltR1/rUrArArUrUrUrCrUrArCrUrGrUrUrGrUrArGrA |
| sgRNA | | sgRNA-G2 | rUrGrCrCrArCrCrUrCrUrCrCrArUrCrCrUrCrUrUrGr |
| targeting | | | CrU/AltR2/ |
| AAVS1 | | | |
| MG29-1 | 4553 | MG29-1-AAVS1- | /AltR1/rUrArArUrUrUrCrUrArCrUrGrUrUrGrUrArGrA |
| sgRNA | | sgRNA-H2 | rUrUrUrUrGrCrCrUrGrGrArCrArCrCrCrCrGrUrUrCr |
| targeting | | | UrC/AltR2/ |
| AAVS1 | | | |
| MG29-1 | 4554 | MG29-1-AAVS1- | /AltR1/rUrArArUrUrUrCrUrArCrUrGrUrUrGrUrArGrA |
| sgRNA | | sgRNA-A3 | rUrCrCrUrGrGrArCrArCrCrCrCrGrUrUrCrUrCrCrUr |
| targeting | | | GrU/AltR2/ |
| AAVS1 | | | |
| MG29-1 | 4555 | MG29-1-AAVS1- | /AltR1/rUrArArUrUrUrCrUrArCrUrGrUrUrGrUrArGrA |
| sgRNA | | sgRNA-B3 | rUrArUrUrUrGrGrGrCrArGrCrUrCrCrCrCrUrArCrCr |
| targeting | | | CrC/AltR2/ |
| AAVS1 | | | |
| MG29-1 | 4556 | MG29-1-AAVS1- | /AltR1/rUrArArUrUrUrCrUrArCrUrGrUrUrGrUrArGrA |
| sgRNA | | sgRNA-C3 | rUrGrGrCrArGrCrUrCrCrCrCrUrArCrCrCrCrCrCrUr |
| targeting | | | UrA/AltR2/ |
| AAVS1 | | | |
| MG29-1 | 4557 | MG29-1-AAVS1- | /AltR1/rUrArArUrUrUrCrUrArCrUrGrUrUrGrUrArGrA |
| sgRNA | | sgRNA-D3 | rUrCrUrGrCrCrUrCrCrArGrGrGrArUrCrCrUrGrUrGr |
| targeting | | | UrC/AltR2/ |
| AAVS1 | | | |
| MG29-1 | 4558 | MG29-1-AAVS1- | /AltR1/rUrArArUrUrUrCrUrArCrUrGrUrUrGrUrArGrA |
| sgRNA | | sgRNA-E3 | rUrArUrCrUrGrUrCrCrCrCrUrCrCrArCrCrCrCrArCr |
| targeting | | | ArG/AltR2/ |
| AAVS1 | | | |
| MG29-1 | 4559 | MG29-1-AAVS1- | /AltR1/rUrArArUrUrUrCrUrArCrUrGrUrUrGrUrArGrA |
| sgRNA | | sgRNA-F3 | rUrUrCrUrGrUrCrCrCrCrUrCrCrArCrCrCrCrArCrAr |
| targeting | | | GrU/AltR2/ |
| AAVS1 | | | |
| MG29-1 | 4560 | MG29-1-AAVS1- | /AltR1/rUrArArUrUrUrCrUrArCrUrGrUrUrGrUrArGrA |
| sgRNA | | sgRNA-G3 | rUrCrUrGrGrArGrCrCrArUrCrUrCrUrCrUrCrCrUrUr |
| targeting | | | GrC/AltR2/ |
| AAVS1 | | | |
| MG29-1 | 4561 | MG29-1-AAVS1- | /AltR1/rUrArArUrUrUrCrUrArCrUrGrUrUrGrUrArGrA |
| sgRNA | | sgRNA-H3 | rUrCrUrUrArCrGrArUrGrGrArGrCrCrArGrArGrArGr |
| targeting | | | GrA/AltR2/ |
| AAVS1 | | | |
| MG29-1 | 4562 | MG29-1-AAVS1- | /AltR1/rUrArArUrUrUrCrUrArCrUrGrUrUrGrUrArGrA |
| sgRNA | | sgRNA-A4 | rUrArCrUrGrArUrCrCrUrGrGrUrGrCrUrGrCrArGrCr |
| targeting | | | UrU/AltR2/ |
| AAVS1 | | | |
| MG29-1 | 4563 | MG29-1-AAVS1- | /AltR1/rUrArArUrUrUrCrUrArCrUrGrUrUrGrUrArGrA |
| sgRNA | | sgRNA-B4 | rUrGrArArArArArCrArArArArUrCrArGrArArUrArAr |
| targeting | | | GrU/AltR2/ |
| AAVS1 | | | |
| MG29-1 | 4564 | MG29-1-AAVS1- | /AltR1/rUrArArUrUrUrCrUrArCrUrGrUrUrGrUrArGrA |
| sgRNA | | sgRNA-C4 | rUrGrCrUrCrUrUrCrArCrCrUrUrUrCrUrArGrUrCrCr |
| targeting | | | CrC/AltR2/ |
| AAVS1 | | | |
| MG29-1 | 4565 | MG29-1-AAVS1- | /AltR1/rUrArArUrUrUrCrUrArCrUrGrUrUrGrUrArGrA |
| sgRNA | | sgRNA-D4 | rUrUrArGrUrCrCrCrCrArArUrUrUrArUrArUrUrGrUr |
| targeting | | | UrC/AltR2/ |
| AAVS1 | | | |
| MG29-1 | 4566 | MG29-1-AAVS1- | /AltR1/rUrArArUrUrUrCrUrArCrUrGrUrUrGrUrArGrA |
| sgRNA | | sgRNA-E4 | rUrUrArUrUrGrUrUrCrCrUrCrCrGrUrGrCrGrUrCrAr |
| targeting | | | GrU/AltR2/ |
| AAVS1 | | | |
| MG29-1 | 4567 | MG29-1-AAVS1- | /AltR1/rUrArArUrUrUrCrUrArCrUrGrUrUrGrUrArGrA |
| sgRNA | | sgRNA-F4 | rUrArCrCrUrGrUrGrArGrArUrArArGrGrCrCrArGrUr |
| targeting | | | ArG/AltR2/ |
| AAVS1 | | | |
| MG29-1 | 4568 | MG29-1-AAVS1- | /AltR1/rUrArArUrUrUrCrUrArCrUrGrUrUrGrUrArGrA |
| sgRNA | | sgRNA-G4 | rUrCrCrUrGrUrGrArGrArUrArArGrGrCrCrArGrUrAr |
| targeting | | | GrC/AltR2/ |
| AAVS1 | | | |
| DNA sequence | 4569 | MG29-1-AAVS1- | GTTTTTCCAAACTGCTTCTCCT |
| of AAVS1 | | target site-A1 | |
| target site | | | |
| DNA sequence | 4570 | MG29-1-AAVS1- | TTTTTCCAAACTGCTTCTCCTC |
| of AAVS1 | | target site-B1 | |
| target site | | | |
| DNA sequence | 4571 | MG29-1-AAVS1- | TCCAAACTGCTTCTCCTCTTGG |
| of AAVS1 | | target site-C1 | |
| target site | | | |
| DNA sequence | 4572 | MG29-1-AAVS1- | CCAAACTGCTTCTCCTCTTGGG |
| of AAVS1 | | target site-D1 | |
| target site | | | |
| DNA sequence | 4573 | MG29-1-AAVS1- | CAAACTGCTTCTCCTCTTGGGA |
| of AAVS1 | | target site-E1 | |
| target site | | | |
| DNA sequence | 4574 | MG29-1-AAVS1- | CTGTCACCAATCCTGTCCCTAG |
| of AAVS1 | | target site-F1 | |
| target site | | | |
| DNA sequence | 4575 | MG29-1-AAVS1- | TGTCACCAATCCTGTCCCTAGT |
| of AAVS1 | | target site-G1 | |
| target site | | | |
| DNA sequence | 4576 | MG29-1-AAVS1- | GGGTTGTCCAGAAAAACGGTGA |
| of AAVS1 | | target site-H1 | |
| target site | | | |
| DNA sequence | 4577 | MG29-1-AAVS1- | CCTTCTCCTTCTGGGGCCTGTG |
| of AAVS1 | | target site-A2 | |
| target site | | | |
| DNA sequence | 4578 | MG29-1-AAVS1- | CTTCTCCTTCTGGGGCCTGTGC |
| of AAVS1 | | target site-B2 | |
| target site | | | |
| DNA sequence | 4579 | MG29-1-AAVS1- | TTAGGATGGCCTTCTCCGACGG |
| of AAVS1 | | target site-C2 | |
| target site | | | |
| DNA sequence | 4580 | MG29-1-AAVS1- | TCTGGACAACCCCAAAGTACCC |
| of AAVS1 | | target site-D2 | |
| target site | | | |
| DNA sequence | 4581 | MG29-1-AAVS1- | CTGGACAACCCCAAAGTACCCC |
| of AAVS1 | | target site-E2 | |
| target site | | | |
| DNA sequence | 4582 | MG29-1-AAVS1- | TGGACAACCCCAAAGTACCCCG |
| of AAVS1 | | target site-F2 | |
| target site | | | |
| DNA sequence | 4583 | MG29-1-AAVS1- | GCCACCTCTCCATCCTCTTGCT |
| of AAVS1 | | target site-G2 | |
| target site | | | |
| DNA sequence | 4584 | MG29-1-AAVS1- | TTTGCCTGGACACCCCGTTCTC |
| of AAVS1 | | target site-H2 | |
| target site | | | |
| DNA sequence | 4585 | MG29-1-AAVS1- | CCTGGACACCCCGTTCTCCTGT |
| of AAVS1 | | target site-A3 | |
| target site | | | |
| DNA sequence | 4586 | MG29-1-AAVS1- | ATTTGGGCAGCTCCCCTACCCC |
| of AAVS1 | | target site-B3 | |
| target site | | | |
| DNA sequence | 4587 | MG29-1-AAVS1- | GGCAGCTCCCCTACCCCCCTTA |
| of AAVS1 | | target site-C3 | |
| target site | | | |
| DNA sequence | 4588 | MG29-1-AAVS1- | CTGCCTCCAGGGATCCTGTGTC |
| of AAVS1 | | target site-D3 | |
| target site | | | |
| DNA sequence | 4589 | MG29-1-AAVS1- | ATCTGTCCCCTCCACCCCACAG |
| of AAVS1 | | target site-E3 | |
| target site | | | |
| DNA sequence | 4590 | MG29-1-AAVS1- | TCTGTCCCCTCCACCCCACAGT |
| of AAVS1 | | target site-F3 | |
| target site | | | |
| DNA sequence | 4591 | MG29-1-AAVS1- | CTGGAGCCATCTCTCTCCTTGC |
| of AAVS1 | | target site-G3 | |
| target site | | | |
| DNA sequence | 4592 | MG29-1-AAVS1- | CTTACGATGGAGCCAGAGAGGA |
| of AAVS1 | | target site-H3 | |
| target site | | | |
| DNA sequence | 4593 | MG29-1-AAVS1- | ACTGATCCTGGTGCTGCAGCTT |
| of AAVS1 | | target site-A4 | |
| target site | | | |
| DNA sequence | 4594 | MG29-1-AAVS1- | GAAAAACAAAATCAGAATAAGT |
| of AAVS1 | | target site-B4 | |
| target site | | | |
| DNA sequence | 4595 | MG29-1-AAVS1- | GCTCTTCACCTTTCTAGTCCCC |
| of AAVS1 | | target site-C4 | |
| target site | | | |
| DNA sequence | 4596 | MG29-1-AAVS1- | TAGTCCCCAATTTATATTGTTC |
| of AAVS1 | | target site-D4 | |
| target site | | | |
| DNA sequence | 4597 | MG29-1-AAVS1- | TATTGTTCCTCCGTGCGTCAGT |
| of AAVS1 | | target site-E4 | |
| target site | | | |
| DNA sequence | 4598 | MG29-1-AAVS1- | ACCTGTGAGATAAGGCCAGTAG |
| of AAVS1 | | target site-F4 | |
| target site | | | |
| DNA sequence | 4599 | MG29-1-AAVS1- | CCTGTGAGATAAGGCCAGTAGC |
| of AAVS1 | | target site-G4 | |
| target site | | | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 15—Gene Editing Outcomes at the DNA Level for B2M

Primary T cells were purified from PMBCs using a negative selection kit (Miltenyi) according to the manufacturer's recommendations. Nucleofection of MG29-1 RNPs (106 pmol protein/160 pmol guide) (SEQ ID NOs: 46004675) was performed into T cells (200,000) using the Lonza 4D electroporator. Cells were harvested and genomic DNA prepared five days post-transfection. PCR primers appropriate for use in NGS-based DNA sequencing were generated, optimized, and used to amplify the individual target sequences for each guide RNA (SEQ ID NOs: 4676-4751). The anmplicons were sequenced on an Illumina MiSeq machine and analyzed with a proprietary Python script to measure gene editing (FIG. 61).

TABLE 14D
Sequences of Guide RNAs and Sequences Targeted for Example 41

| | SEQ | | |
|---|---|---|---|
| | ID | | |
| Guide Target | NO | Guide Name | SEQUENCE |
| MG29-1 | 4600 | MG29-1-B2M- | /AltR1/rUrArArUrUrUrCrUrArCrUrGrUrUrGrUrArGrA |
| sgRNA | | sgRNA-A1 | rUrUrGrGrCrCrUrGrGrArGrGrCrUrArUrCrCrArGrCr |
| targeting B2M | | | GrU/AltR2/ |
| MG29-1 | 4601 | MG29-1-B2M- | /AltR1/rUrArArUrUrUrCrUrArCrUrGrUrUrGrUrArGrA |
| sgRNA | | sgRNA-B1 | rUrCrCrCrGrArUrArUrUrCrCrUrCrArGrGrUrArCrUr |
| targeting B2M | | | CrC/AltR2/ |
| MG29-1 | 4602 | MG29-1-B2M- | /AltR1/rUrArArUrUrUrCrUrArCrUrGrUrUrGrUrArGrA |
| sgRNA | | sgRNA-C1 | rUrCrCrGrArUrArUrUrCrCrUrCrArGrGrUrArCrUrCr |
| targeting B2M | | | CrA/AltR2/ |
| MG29-1 | 4603 | MG29-1-B2M- | /AltR1/rUrArArUrUrUrCrUrArCrUrGrUrUrGrUrArGrA |
| sgRNA | | sgRNA-D1 | rUrCrUrCrArCrGrUrCrArUrCrCrArGrCrArGrArGrAr |
| targeting B2M | | | ArU/AltR2/ |
| MG29-1 | 4604 | MG29-1-B2M- | /AltR1/rUrArArUrUrUrCrUrArCrUrGrUrUrGrUrArGrA |
| sgRNA | | sgRNA-E1 | rUrCrUrGrArArUrUrGrCrUrArUrGrUrGrUrCrUrGrGr |
| targeting B2M | | | GrU/AltR2/ |
| MG29-1 | 4605 | MG29-1-B2M- | /AltR1/rUrArArUrUrUrCrUrArCrUrGrUrUrGrUrArGrA |
| sgRNA | | sgRNA-F1 | rUrArUrCrCrArUrCrCrGrArCrArUrUrGrArArGrUrUr |
| targeting B2M | | | GrA/AltR2/ |
| MG29-1 | 4606 | MG29-1-B2M- | /AltR1/rUrArArUrUrUrCrUrArCrUrGrUrUrGrUrArGrA |
| sgRNA | | sgRNA-G1 | rUrArGrCrArArGrGrArCrUrGrGrUrCrUrUrUrCrUrAr |
| targeting B2M | | | UrC/AltR2/ |
| MG29-1 | 4607 | MG29-1-B2M- | /AltR1/rUrArArUrUrUrCrUrArCrUrGrUrUrGrUrArGrA |
| sgRNA | | sgRNA-H1 | rUrUrArUrCrUrCrUrUrGrUrArCrUrArCrArCrUrGrAr |
| targeting B2M | | | ArU/AltR2/ |
| MG29-1 | 4608 | MG29-1-B2M- | /AltR1/rUrArArUrUrUrCrUrArCrUrGrUrUrGrUrArGrA |
| sgRNA | | sgRNA-A2 | rUrUrCrArCrArGrCrCrCrArArGrArUrArGrUrUrArAr |
| targeting B2M | | | GrU/AltR2/ |
| MG29-1 | 4609 | MG29-1-B2M- | /AltR1/rUrArArUrUrUrCrUrArCrUrGrUrUrGrUrArGrA |
| sgRNA | | sgRNA-B2 | rUrUrCrArGrUrGrGrGrGrGrUrGrArArUrUrCrArGrUr |
| targeting B2M | | | GrU/AltR2/ |
| MG29-1 | 4610 | MG29-1-B2M- | /AltR1/rUrArArUrUrUrCrUrArCrUrGrUrUrGrUrArGrA |
| sgRNA | | sgRNA-C2 | rUrCrArGrUrGrGrGrGrGrUrGrArArUrUrCrArGrUrGr |
| targeting B2M | | | UrA/AltR2/ |
| MG29-1 | 4611 | MG29-1-B2M- | /AltR1/rUrArArUrUrUrCrUrArCrUrGrUrUrGrUrArGrA |
| sgRNA | | sgRNA-D2 | rUrArGrUrGrGrGrGrGrUrGrArArUrUrCrArGrUrGrUr |
| targeting B2M | | | ArG/AltR2/ |
| MG29-1 | 4612 | MG29-1-B2M- | /AltR1/rUrArArUrUrUrCrUrArCrUrGrUrUrGrUrArGrA |
| sgRNA | | sgRNA-E2 | rUrUrCrArArUrUrCrUrCrUrCrUrCrCrArUrUrCrUrUr |
| targeting B2M | | | CrA/AltR2/ |
| MG29-1 | 4613 | MG29-1-B2M- | /AltR1/rUrArArUrUrUrCrUrArCrUrGrUrUrGrUrArGrA |
| sgRNA | | sgRNA-F2 | rUrCrArArUrUrCrUrCrUrCrUrCrCrArUrUrCrUrUrCr |
| targeting B2M | | | ArG/AltR2/ |
| MG29-1 | 4614 | MG29-1-B2M- | /AltR1/rUrArArUrUrUrCrUrArCrUrGrUrUrGrUrArGrA |
| sgRNA | | sgRNA-G2 | rUrArArUrUrCrUrCrUrCrUrCrCrArUrUrCrUrUrCrAr |
| targeting B2M | | | GrU/AltR2/ |
| MG29-1 | 4615 | MG29-1-B2M- | /AltR1/rUrArArUrUrUrCrUrArCrUrGrUrUrGrUrArGrA |
| sgRNA | | sgRNA-H2 | rUrArCrUrUrUrCrCrArUrUrCrUrCrUrGrCrUrGrGrAr |
| targeting B2M | | | UrG/AltR2/ |
| MG29-1 | 4616 | MG29-1-B2M- | /AltR1/rUrArArUrUrUrCrUrArCrUrGrUrUrGrUrArGrA |
| sgRNA | | sgRNA-A3 | rUrCrArUrUrCrUrCrUrGrCrUrGrGrArUrGrArCrGrUr |
| targeting B2M | | | GrA/AltR2/ |
| MG29-1 | 4617 | MG29-1-B2M- | /AltR1/rUrArArUrUrUrCrUrArCrUrGrUrUrGrUrArGrA |
| sgRNA | | sgRNA-B3 | rUrUrCrUrCrCrArCrUrGrUrCrUrUrUrUrUrCrArUrAr |
| targeting B2M | | | GrA/AltR2/ |
| MG29-1 | 4618 | MG29-1-B2M- | /AltR1/rUrArArUrUrUrCrUrArCrUrGrUrUrGrUrArGrA |
| sgRNA | | sgRNA-C3 | rUrCrUrCrCrArCrUrGrUrCrUrUrUrUrUrCrArUrArGr |
| targeting B2M | | | ArU/AltR2/ |
| MG29-1 | 4619 | MG29-1-B2M- | /AltR1/rUrArArUrUrUrCrUrArCrUrGrUrUrGrUrArGrA |
| sgRNA | | sgRNA-D3 | rUrUrCrCrArCrUrGrUrCrUrUrUrUrUrCrArUrArGrAr |
| targeting B2M | | | UrC/AltR2/ |
| MG29-1 | 4620 | MG29-1-B2M- | /AltR1/rUrArArUrUrUrCrUrArCrUrGrUrUrGrUrArGrA |
| sgRNA | | sgRNA-E3 | rUrUrCrArUrArGrArUrCrGrArGrArCrArUrGrUrArAr |
| targeting B2M | | | GrC/AltR2/ |
| MG29-1 | 4621 | MG29-1-B2M- | /AltR1/rUrArArUrUrUrCrUrArCrUrGrUrUrGrUrArGrA |
| sgRNA | | sgRNA-F3 | rUrCrArUrArGrArUrCrGrArGrArCrArUrGrUrArArGr |
| targeting B2M | | | CrA/AltR2/ |
| MG29-1 | 4622 | MG29-1-B2M- | /AltR1/rUrArArUrUrUrCrUrArCrUrGrUrUrGrUrArGrA |
| sgRNA | | sgRNA-G3 | rUrUrGrGrCrCrUrGrGrArGrGrCrUrArUrCrCrArGrCr |
| targeting B2M | | | GrU/AltR2/ |
| MG29-1 | 4623 | MG29-1-B2M- | /AltR1/rUrArArUrUrUrCrUrArCrUrGrUrUrGrUrArGrA |
| sgRNA | | sgRNA-H3 | rUrGrGrCrGrGrGrGrArGrCrArGrGrGrGrArGrArCrCr |
| targeting B2M | | | UrU/AltR2/ |
| MG29-1 | 4624 | MG29-1-B2M- | /AltR1/rUrArArUrUrUrCrUrArCrUrGrUrUrGrUrArGrA |
| sgRNA | | sgRNA-A4 | rUrGrCrCrUrArCrGrGrCrGrArCrGrGrGrArGrGrGrUr |
| targeting B2M | | | CrG/AltR2/ |
| MG29-1 | 4625 | MG29-1-B2M- | /AltR1/rUrArArUrUrUrCrUrArCrUrGrUrUrGrUrArGrA |
| sgRNA | | sgRNA-B4 | rUrGrGrGrCrGrUrCrGrArUrArArGrCrGrUrCrArGrAr |
| targeting B2M | | | GrC/AltR2/ |
| MG29-1 | 4626 | MG29-1-B2M- | /AltR1/rUrArArUrUrUrCrUrArCrUrGrUrUrGrUrArGrA |
| sgRNA | | sgRNA-C4 | rUrUrCrUrUrCrCrGrCrUrCrUrUrUrCrGrCrGrGrGrGr |
| targeting B2M | | | CrC/AltR2/ |
| MG29-1 | 4627 | MG29-1-B2M- | /AltR1/rUrArArUrUrUrCrUrArCrUrGrUrUrGrUrArGrA |
| sgRNA | | sgRNA-D4 | rUrGrCrGrGrGrGrCrCrUrCrUrGrGrCrUrCrCrCrCrCr |
| targeting B2M | | | ArG/AltR2/ |
| MG29-1 | 4628 | MG29-1-B2M- | /AltR1/rUrArArUrUrUrCrUrArCrUrGrUrUrGrUrArGrA |
| sgRNA | | sgRNA-E4 | rUrUrGrArArCrGrCrGrUrGrGrArGrGrGrGrCrGrCrUr |
| targeting B2M | | | UrG/AltR2/ |
| MG29-1 | 4629 | MG29-1-B2M- | /AltR1/rUrArArUrUrUrCrUrArCrUrGrUrUrGrUrArGrA |
| sgRNA | | sgRNA-F4 | rUrCrUrCrCrCrCrArCrGrGrUrGrUrGrGrCrCrCrCrAr |
| targeting B2M | | | CrA/AltR2/ |
| MG29-1 | 4630 | MG29-1-B2M- | /AltR1/rUrArArUrUrUrCrUrArCrUrGrUrUrGrUrArGrA |
| sgRNA | | sgRNA-G4 | rUrGrGrArCrGrArGrCrCrUrArCrCrCrGrUrCrCrCrCr |
| targeting B2M | | | CrA/AltR2/ |
| MG29-1 | 4631 | MG29-1-B2M- | /AltR1/rUrArArUrUrUrCrUrArCrUrGrUrUrGrUrArGrA |
| sgRNA | | sgRNA-H4 | rUrUrCrCrCrGrArCrCrCrUrCrCrCrGrUrCrGrCrCrGr |
| targeting B2M | | | UrA/AltR2/ |
| MG29-1 | 4632 | MG29-1-B2M- | /AltR1/rUrArArUrUrUrCrUrArCrUrGrUrUrGrUrArGrA |
| sgRNA | | sgRNA-A5 | rUrGrCrGrGrGrArGrCrGrCrArUrGrCrCrUrUrUrUrGr |
| targeting B2M | | | GrC/AltR2/ |
| MG29-1 | 4633 | MG29-1-B2M- | /AltR1/rUrArArUrUrUrCrUrArCrUrGrUrUrGrUrArGrA |
| sgRNA | | sgRNA-B5 | rUrCrGrGrGrArGrCrGrCrArUrGrCrCrUrUrUrUrGrGr |
| targeting B2M | | | CrU/AltR2/ |
| MG29-1 | 4634 | MG29-1-B2M- | /AltR1/rUrArArUrUrUrCrUrArCrUrGrUrUrGrUrArGrA |
| sgRNA | | sgRNA-C5 | rUrGrGrCrUrGrUrArArUrUrCrGrUrGrCrArUrUrUrUr |
| targeting B2M | | | UrU/AltR2/ |
| MG29-1 | 4635 | MG29-1-B2M- | /AltR1/rUrArArUrUrUrCrUrArCrUrGrUrUrGrUrArGrA |
| sgRNA | | sgRNA-D5 | rUrGrCrUrGrUrArArUrUrCrGrUrGrCrArUrUrUrUrUr |
| targeting B2M | | | UrU/AltR2/ |
| MG29-1 | 4636 | MG29-1-B2M- | /AltR1/rUrArArUrUrUrCrUrArCrUrGrUrUrGrUrArGrA |
| sgRNA | | sgRNA-E5 | rUrUrUrUrUrUrArArGrArArArArArCrGrCrCrUrGrCr |
| targeting B2M | | | CrU/AltR2/ |
| MG29-1 | 4637 | MG29-1-B2M- | /AltR1/rUrArArUrUrUrCrUrArCrUrGrUrUrGrUrArGrA |
| sgRNA | | sgRNA-F5 | rUrUrUrUrUrArArGrArArArArArCrGrCrCrUrGrCrCr |
| targeting B2M | | | UrU/AltR2/ |
| MG29-1 | 4638 | MG29-1-B2M- | /AltR1/rUrArArUrUrUrCrUrArCrUrGrUrUrGrUrArGrA |
| sgRNA | | sgRNA-G5 | rUrUrUrUrArArGrArArArArArCrGrCrCrUrGrCrCrUr |
| targeting B2M | | | UrC/AltR2/ |
| MG29-1 | 4639 | MG29-1-B2M- | /AltR1/rUrArArUrUrUrCrUrArCrUrGrUrUrGrUrArGrA |
| sgRNA | | sgRNA-H5 | rUrUrUrArArGrArArArArArCrGrCrCrUrGrCrCrUrUr |
| targeting B2M | | | CrU/AltR2/ |
| MG29-1 | 4640 | MG29-1-B2M- | /AltR1/rUrArArUrUrUrCrUrArCrUrGrUrUrGrUrArGrA |
| sgRNA | | sgRNA-A6 | rUrUrArArGrArArArArArCrGrCrCrUrGrCrCrUrUrCr |
| targeting B2M | | | UrG/AltR2/ |
| MG29-1 | 4641 | MG29-1-B2M- | /AltR1/rUrArArUrUrUrCrUrArCrUrGrUrUrGrUrArGrA |
| sgRNA | | sgRNA-B6 | rUrArArGrArArArArArCrGrCrCrUrGrCrCrUrUrCrUr |
| targeting B2M | | | GrC/AltR2/ |
| MG29-1 | 4642 | MG29-1-B2M- | /AltR1/rUrArArUrUrUrCrUrArCrUrGrUrUrGrUrArGrA |
| sgRNA | | sgRNA-C6 | rUrArGrArArArArArCrGrCrCrUrGrCrCrUrUrCrUrGr |
| targeting B2M | | | CrG/AltR2/ |
| MG29-1 | 4643 | MG29-1-B2M- | /AltR1/rUrArArUrUrUrCrUrArCrUrGrUrUrGrUrArGrA |
| sgRNA | | sgRNA-D6 | rUrCrGrUrArCrArGrArGrGrGrCrUrUrCrCrUrCrUrUr |
| targeting B2M | | | UrG/AltR2/ |
| MG29-1 | 4644 | MG29-1-B2M- | /AltR1/rUrArArUrUrUrCrUrArCrUrGrUrUrGrUrArGrA |
| sgRNA | | sgRNA-E6 | rUrGrUrArCrArGrArGrGrGrCrUrUrCrCrUrCrUrUrUr |
| targeting B2M | | | GrG/AltR2/ |
| MG29-1 | 4645 | MG29-1-B2M- | /AltR1/rUrArArUrUrUrCrUrArCrUrGrUrUrGrUrArGrA |
| sgRNA | | sgRNA-F6 | rUrGrCrUrCrUrUrUrGrCrCrUrGrGrUrUrGrUrUrUrCr |
| targeting B2M | | | CrA/AltR2/ |
| MG29-1 | 4646 | MG29-1-B2M- | /AltR1/rUrArArUrUrUrCrUrArCrUrGrUrUrGrUrArGrA |
| sgRNA | | sgRNA-G6 | rUrCrCrUrGrGrUrUrGrUrUrUrCrCrArArGrArUrGrUr |
| targeting B2M | | | ArC/AltR2/ |
| MG29-1 | 4647 | MG29-1-B2M- | /AltR1/rUrArArUrUrUrCrUrArCrUrGrUrUrGrUrArGrA |
| sgRNA | | sgRNA-H6 | rUrCrArArGrArUrGrUrArCrUrGrUrGrCrCrUrCrUrUr |
| targeting B2M | | | ArC/AltR2/ |
| MG29-1 | 4648 | MG29-1-B2M- | /AltR1/rUrArArUrUrUrCrUrArCrUrGrUrUrGrUrArGrA |
| sgRNA | | sgRNA-A7 | rUrCrArArArArCrCrGrArArArGrUrArArGrArGrGrCr |
| targeting B2M | | | ArC/AltR2/ |
| MG29-1 | 4649 | MG29-1-B2M- | /AltR1/rUrArArUrUrUrCrUrArCrUrGrUrUrGrUrArGrA |
| sgRNA | | sgRNA-B7 | rUrArArArArCrCrGrArArArGrUrArArGrArGrGrCrAr |
| targeting B2M | | | CrA/AltR2/ |
| MG29-1 | 4650 | MG29-1-B2M- | /AltR1/rUrArArUrUrUrCrUrArCrUrGrUrUrGrUrArGrA |
| sgRNA | | sgRNA-C7 | rUrCrUrCrUrGrGrArGrArArUrCrUrCrArCrGrCrArGr |
| targeting B2M | | | ArA/AltR2/ |
| MG29-1 | 4651 | MG29-1-B2M- | /AltR1/rUrArArUrUrUrCrUrArCrUrGrUrUrGrUrArGrA |
| sgRNA | | sgRNA-D7 | rUrUrCrUrUrArArArArArArArArArUrGrCrArCrGrAr |
| targeting B2M | | | ArU/AltR2/ |
| MG29-1 | 4652 | MG29-1-B2M- | /AltR1/rUrArArUrUrUrCrUrArCrUrGrUrUrGrUrArGrA |
| sgRNA | | sgRNA-E7 | rUrCrUrUrArArArArArArArArArUrGrCrArCrGrArAr |
| targeting B2M | | | UrU/AltR2/ |
| MG29-1 | 4653 | MG29-1-B2M- | /AltR1/rUrArArUrUrUrCrUrArCrUrGrUrUrGrUrArGrA |
| sgRNA | | sgRNA-F7 | rUrUrUrArArArArArArArArArUrGrCrArCrGrArArUr |
| targeting B2M | | | UrA/AltR2/ |
| MG29-1 | 4654 | MG29-1-B2M- | /AltR1/rUrArArUrUrUrCrUrArCrUrGrUrUrGrUrArGrA |
| sgRNA | | sgRNA-G7 | rUrUrUrCrUrUrCrArArArArUrGrGrArGrGrUrGrGrCr |
| targeting B2M | | | UrU/AltR2/ |
| MG29-1 | 4655 | MG29-1-B2M- | /AltR1/rUrArArUrUrUrCrUrArCrUrGrUrUrGrUrArGrA |
| sgRNA | | sgRNA-H7 | rUrGrCrCrArGrArGrUrGrGrArArArUrGrGrArArUrUr |
| targeting B2M | | | GrG/AltR2/ |
| MG29-1 | 4656 | MG29-1-B2M- | /AltR1/rUrArArUrUrUrCrUrArCrUrGrUrUrGrUrArGrA |
| sgRNA | | sgRNA-A8 | rUrGrArGrUrArCrCrUrGrArGrGrArArUrArUrCrGrGr |
| targeting B2M | | | GrA/AltR2/ |
| MG29-1 | 4657 | MG29-1-B2M- | /AltR1/rUrArArUrUrUrCrUrArCrUrGrUrUrGrUrArGrA |
| sgRNA | | sgRNA-B8 | rUrArCrUrUrGrGrGrGrCrUrArArCrUrUrGrGrUrGrUr |
| targeting B2M | | | CrA/AltR2/ |
| MG29-1 | 4658 | MG29-1-B2M- | /AltR1/rUrArArUrUrUrCrUrArCrUrGrUrUrGrUrArGrA |
| sgRNA | | sgRNA-C8 | rUrCrArUrUrUrGrGrUrCrArUrCrGrArUrUrUrCrUrCr |
| targeting B2M | | | CrC/AltR2/ |
| MG29-1 | 4659 | MG29-1-B2M- | /AltR1/rUrArArUrUrUrCrUrArCrUrGrUrUrGrUrArGrA |
| sgRNA | | sgRNA-D8 | rUrGrUrCrArUrCrGrArUrUrUrCrUrCrCrCrArArUrUr |
| targeting B2M | | | CrC/AltR2/ |
| MG29-1 | 4660 | MG29-1-B2M- | /AltR1/rUrArArUrUrUrCrUrArCrUrGrUrUrGrUrArGrA |
| sgRNA | | sgRNA-E8 | rUrUrCrCrCrArArUrUrCrCrArUrUrUrCrCrArCrUrCr |
| targeting B2M | | | UrG/AltR2/ |
| MG29-1 | 4661 | MG29-1-B2M- | /AltR1/rUrArArUrUrUrCrUrArCrUrGrUrUrGrUrArGrA |
| sgRNA | | sgRNA-F8 | rUrCrArCrUrCrUrGrGrCrCrArArArUrGrArGrCrUrUr |
| targeting B2M | | | CrC/AltR2/ |
| MG29-1 | 4662 | MG29-1-B2M- | /AltR1/rUrArArUrUrUrCrUrArCrUrGrUrUrGrUrArGrA |
| sgRNA | | sgRNA-G8 | rUrGrArArGrArArUrArArArCrCrGrUrGrArCrUrUrGr |
| targeting B2M | | | GrU/AltR2/ |
| MG29-1 | 4663 | MG29-1-B2M- | /AltR1/rUrArArUrUrUrCrUrArCrUrGrUrUrGrUrArGrA |
| sgRNA | | sgRNA-H8 | rUrArArGrArArUrArArArCrCrGrUrGrArCrUrUrGrGr |
| targeting B2M | | | UrA/AltR2/ |
| MG29-1 | 4664 | MG29-1-B2M- | /AltR1/rUrArArUrUrUrCrUrArCrUrGrUrUrGrUrArGrA |
| sgRNA | | sgRNA-A9 | rUrCrCrUrCrArUrArArUrUrCrCrUrCrUrArUrArCrAr |
| targeting B2M | | | UrG/AltR2/ |
| MG29-1 | 4665 | MG29-1-B2M- | /AltR1/rUrArArUrUrUrCrUrArCrUrGrUrUrGrUrArGrA |
| sgRNA | | sgRNA-B9 | rUrUrUrGrUrUrUrUrUrUrUrUrCrUrArGrCrArGrArUr |
| targeting B2M | | | UrU/AltR2/ |
| MG29-1 | 4666 | MG29-1-B2M- | /AltR1/rUrArArUrUrUrCrUrArCrUrGrUrUrGrUrArGrA |
| sgRNA | | sgRNA-C9 | rUrUrGrUrUrUrUrUrUrUrUrCrUrArGrCrArGrArUrUr |
| targeting B2M | | | UrC/AltR2/ |
| MG29-1 | 4667 | MG29-1-B2M- | /AltR1/rUrArArUrUrUrCrUrArCrUrGrUrUrGrUrArGrA |
| sgRNA | | sgRNA-D9 | rUrGrUrUrUrUrUrUrUrUrCrUrArGrCrArGrArUrUrUr |
| targeting B2M | | | CrU/AltR2/ |
| MG29-1 | 4668 | MG29-1-B2M- | /AltR1/rUrArArUrUrUrCrUrArCrUrGrUrUrGrUrArGrA |
| sgRNA | | sgRNA-E9 | rUrUrUrUrUrUrUrUrUrCrUrArGrCrArGrArUrUrUrCr |
| targeting B2M | | | UrA/AltR2/ |
| MG29-1 | 4669 | MG29-1-B2M- | /AltR1/rUrArArUrUrUrCrUrArCrUrGrUrUrGrUrArGrA |
| sgRNA | | sgRNA-F9 | rUrUrUrUrUrCrUrArGrCrArGrArUrUrUrCrUrArGrCr |
| targeting B2M | | | ArG/AltR2/ |
| MG29-1 | 4670 | MG29-1-B2M- | /AltR1/rUrArArUrUrUrCrUrArCrUrGrUrUrGrUrArGrA |
| sgRNA | | sgRNA-G9 | rUrUrUrUrCrUrArGrCrArGrArUrUrUrCrUrArGrCrAr |
| targeting B2M | | | GrU/AltR2/ |
| MG29-1 | 4671 | MG29-1-B2M- | /AltR1/rUrArArUrUrUrCrUrArCrUrGrUrUrGrUrArGrA |
| sgRNA | | sgRNA-H9 | rUrUrUrCrUrArGrCrArGrArUrUrUrCrUrArGrCrArGr |
| targeting B2M | | | UrA/AltR2/ |
| MG29-1 | 4672 | MG29-1-B2M- | /AltR1/rUrArArUrUrUrCrUrArCrUrGrUrUrGrUrArGrA |
| sgRNA | | sgRNA-A10 | rUrUrCrUrArGrCrArGrArUrUrUrCrUrArGrCrArGrUr |
| targeting B2M | | | ArU/AltR2/ |
| MG29-1 | 4673 | MG29-1-B2M- | /AltR1/rUrArArUrUrUrCrUrArCrUrGrUrUrGrUrArGrA |
| sgRNA | | sgRNA-B10 | rUrCrUrArGrCrArGrArUrUrUrCrUrArGrCrArGrUrAr |
| targeting B2M | | | UrC/AltR2/ |
| MG29-1 | 4674 | MG29-1-B2M- | /AltR1/rUrArArUrUrUrCrUrArCrUrGrUrUrGrUrArGrA |
| sgRNA | | sgRNA-C10 | rUrUrArGrCrArGrArUrUrUrCrUrArGrCrArGrUrArUr |
| targeting B2M | | | CrU/AltR2/ |
| MG29-1 | 4675 | MG29-1-B2M- | /AltR1/rUrArArUrUrUrCrUrArCrUrGrUrUrGrUrArGrA |
| sgRNA | | sgRNA-D10 | rUrUrArGrCrArGrUrArUrCrUrUrCrUrGrUrCrArCrUr |
| targeting B2M | | | GrG/AltR2/ |
| DNA sequence | 4676 | MG29-1-B2M- | TGGCCTGGAGGCTATCCAGCGT |
| of B2M target | | target site-A1 | |
| site | | | |
| DNA sequence | 4677 | MG29-1-B2M- | CCCGATATTCCTCAGGTACTCC |
| of B2M target | | target site-B1 | |
| site | | | |
| DNA sequence | 4678 | MG29-1-B2M- | CCGATATTCCTCAGGTACTCCA |
| of B2M target | | target site-C1 | |
| site | | | |
| DNA sequence | 4679 | MG29-1-B2M- | CTCACGTCATCCAGCAGAGAAT |
| of B2M target | | target site-D1 | |
| site | | | |
| DNA sequence | 4680 | MG29-1-B2M- | CTGAATTGCTATGTGTCTGGGT |
| of B2M target | | target site-E1 | |
| site | | | |
| DNA sequence | 4681 | MG29-1-B2M- | ATCCATCCGACATTGAAGTTGA |
| of B2M target | | target site-F1 | |
| site | | | |
| DNA sequence | 4682 | MG29-1-B2M- | AGCAAGGACTGGTCTTTCTATC |
| of B2M target | | target site-G1 | |
| site | | | |
| DNA sequence | 4683 | MG29-1-B2M- | TATCTCTTGTACTACACTGAAT |
| of B2M target | | target site-H1 | |
| site | | | |
| DNA sequence | 4684 | MG29-1-B2M- | TCACAGCCCAAGATAGTTAAGT |
| of B2M target | | target site-A2 | |
| site | | | |
| DNA sequence | 4685 | MG29-1-B2M- | TCAGTGGGGGTGAATTCAGTGT |
| of B2M target | | target site-B2 | |
| site | | | |
| DNA sequence | 4686 | MG29-1-B2M- | CAGTGGGGGTGAATTCAGTGTA |
| of B2M target | | target site-C2 | |
| site | | | |
| DNA sequence | 4687 | MG29-1-B2M- | AGTGGGGGTGAATTCAGTGTAG |
| of B2M target | | target site-D2 | |
| site | | | |
| DNA sequence | 4688 | MG29-1-B2M- | TCAATTCTCTCTCCATTCTTCA |
| of B2M target | | target site-E2 | |
| site | | | |
| DNA sequence | 4689 | MG29-1-B2M- | CAATTCTCTCTCCATTCTTCAG |
| of B2M target | | target site-F2 | |
| site | | | |
| DNA sequence | 4690 | MG29-1-B2M- | AATTCTCTCTCCATTCTTCAGT |
| of B2M target | | target site-G2 | |
| site | | | |
| DNA sequence | 4691 | MG29-1-B2M- | ACTTTCCATTCTCTGCTGGATG |
| of B2M target | | target site-H2 | |
| site | | | |
| DNA sequence | 4692 | MG29-1-B2M- | CATTCTCTGCTGGATGACGTGA |
| of B2M target | | target site-A3 | |
| site | | | |
| DNA sequence | 4693 | MG29-1-B2M- | TCTCCACTGTCTTTTTCATAGA |
| of B2M target | | target site-B3 | |
| site | | | |
| DNA sequence | 4694 | MG29-1-B2M- | CTCCACTGTCTTTTTCATAGAT |
| of B2M target | | target site-C3 | |
| site | | | |
| DNA sequence | 4695 | MG29-1-B2M- | TCCACTGTCTTTTTCATAGATC |
| of B2M target | | target site-D3 | |
| site | | | |
| DNA sequence | 4696 | MG29-1-B2M- | TCATAGATCGAGACATGTAAGC |
| of B2M target | | target site-E3 | |
| site | | | |
| DNA sequence | 4697 | MG29-1-B2M- | CATAGATCGAGACATGTAAGCA |
| of B2M target | | target site-F3 | |
| site | | | |
| DNA sequence | 4698 | MG29-1-B2M- | TGGCCTGGAGGCTATCCAGCGT |
| of B2M target | | target site-G3 | |
| site | | | |
| DNA sequence | 4699 | MG29-1-B2M- | GGCGGGGAGCAGGGGAGACCTT |
| of B2M target | | target site-H3 | |
| site | | | |
| DNA sequence | 4700 | MG29-1-B2M- | GCCTACGGCGACGGGAGGGTCG |
| of B2M target | | target site-A4 | |
| site | | | |
| DNA sequence | 4701 | MG29-1-B2M- | GGGCGTCGATAAGCGTCAGAGC |
| of B2M target | | target site-B4 | |
| site | | | |
| DNA sequence | 4702 | MG29-1-B2M- | TCTTCCGCTCTTTCGCGGGGCC |
| of B2M target | | target site-C4 | |
| site | | | |
| DNA sequence | 4703 | MG29-1-B2M- | GCGGGGCCTCTGGCTCCCCCAG |
| of B2M target | | target site-D4 | |
| site | | | |
| DNA sequence | 4704 | MG29-1-B2M- | TGAACGCGTGGAGGGGCGCTTG |
| of B2M target | | target site-E4 | |
| site | | | |
| DNA sequence | 4705 | MG29-1-B2M- | CTCCCCACGGTGTGGCCCCACA |
| of B2M target | | target site-F4 | |
| site | | | |
| DNA sequence | 4706 | MG29-1-B2M- | GGACGAGCCTACCCGTCCCCCA |
| of B2M target | | target site-G4 | |
| site | | | |
| DNA sequence | 4707 | MG29-1-B2M- | TCCCGACCCTCCCGTCGCCGTA |
| of B2M target | | target site-H4 | |
| site | | | |
| DNA sequence | 4708 | MG29-1-B2M- | GCGGGAGCGCATGCCTTTTGGC |
| of B2M target | | target site-A5 | |
| site | | | |
| DNA sequence | 4709 | MG29-1-B2M- | CGGGAGCGCATGCCTTTTGGCT |
| of B2M target | | target site-B5 | |
| site | | | |
| DNA sequence | 4710 | MG29-1-B2M- | GGCTGTAATTCGTGCATTTTTT |
| of B2M target | | target site-C5 | |
| site | | | |
| DNA sequence | 4711 | MG29-1-B2M- | GCTGTAATTCGTGCATTTTTTT |
| of B2M target | | target site-D5 | |
| site | | | |
| DNA sequence | 4712 | MG29-1-B2M- | TTTTTAAGAAAAACGCCTGCCT |
| of B2M target | | target site-E5 | |
| site | | | |
| DNA sequence | 4713 | MG29-1-B2M- | TTTTAAGAAAAACGCCTGCCTT |
| of B2M target | | target site-F5 | |
| site | | | |
| DNA sequence | 4714 | MG29-1-B2M- | TTTAAGAAAAACGCCTGCCTTC |
| of B2M target | | target site-G5 | |
| site | | | |
| DNA sequence | 4715 | MG29-1-B2M- | TTAAGAAAAACGCCTGCCTTCT |
| of B2M target | | target site-H5 | |
| site | | | |
| DNA sequence | 4716 | MG29-1-B2M- | TAAGAAAAACGCCTGCCTTCTG |
| of B2M target | | target site-A6 | |
| site | | | |
| DNA sequence | 4717 | MG29-1-B2M- | AAGAAAAACGCCTGCCTTCTGC |
| of B2M target | | target site-B6 | |
| site | | | |
| DNA sequence | 4718 | MG29-1-B2M- | AGAAAAACGCCTGCCTTCTGCG |
| of B2M target | | target site-C6 | |
| site | | | |
| DNA sequence | 4719 | MG29-1-B2M- | CGTACAGAGGGCTTCCTCTTTG |
| of B2M target | | target site-D6 | |
| site | | | |
| DNA sequence | 4720 | MG29-1-B2M- | GTACAGAGGGCTTCCTCTTTGG |
| of B2M target | | target site-E6 | |
| site | | | |
| DNA sequence | 4721 | MG29-1-B2M- | GCTCTTTGCCTGGTTGTTTCCA |
| of B2M target | | target site-F6 | |
| site | | | |
| DNA sequence | 4722 | MG29-1-B2M- | CCTGGTTGTTTCCAAGATGTAC |
| of B2M target | | target site-G6 | |
| site | | | |
| DNA sequence | 4723 | MG29-1-B2M- | CAAGATGTACTGTGCCTCTTAC |
| of B2M target | | target site-H6 | |
| site | | | |
| DNA sequence | 4724 | MG29-1-B2M- | CAAAACCGAAAGTAAGAGGCAC |
| of B2M target | | target site-A7 | |
| site | | | |
| DNA sequence | 4725 | MG29-1-B2M- | AAAACCGAAAGTAAGAGGCACA |
| of B2M target | | target site-B7 | |
| site | | | |
| DNA sequence | 4726 | MG29-1-B2M | CTCTGGAGAATCTCACGCAGAA |
| of B2M target | | target site-C7 | |
| site | | | |
| DNA sequence | 4727 | MG29-1-B2M- | TCTTAAAAAAAAATGCACGAAT |
| of B2M target | | target site-D7 | |
| site | | | |
| DNA sequence | 4728 | MG29-1-B2M- | CTTAAAAAAAAATGCACGAATT |
| of B2M target | | target site-E7 | |
| site | | | |
| DNA sequence | 4729 | MG29-1-B2M- | TTAAAAAAAAATGCACGAATTA |
| of B2M target | | target site-F7 | |
| site | | | |
| DNA sequence | 4730 | MG29-1-B2M- | TTCTTCAAAATGGAGGTGGCTT |
| of B2M target | | target site-G7 | |
| site | | | |
| DNA sequence | 4731 | MG29-1-B2M- | GCCAGAGTGGAAATGGAATTGG |
| of B2M target | | target site-H7 | |
| site | | | |
| DNA sequence | 4732 | MG29-1-B2M- | GAGTACCTGAGGAATATCGGGA |
| of B2M target | | target site-A8 | |
| site | | | |
| DNA sequence | 4733 | MG29-1-B2M- | ACTTGGGGCTAACTTGGTGTCA |
| of B2M target | | target site-B8 | |
| site | | | |
| DNA sequence | 4734 | MG29-1-B2M- | CATTTGGTCATCGATTTCTCCC |
| of B2M target | | target site-C8 | |
| site | | | |
| DNA sequence | 4735 | MG29-1-B2M- | GTCATCGATTTCTCCCAATTCC |
| of B2M target | | target site-D8 | |
| site | | | |
| DNA sequence | 4736 | MG29-1-B2M- | TCCCAATTCCATTTCCACTCTG |
| of B2M target | | target site-E8 | |
| site | | | |
| DNA sequence | 4737 | MG29-1-B2M- | CACTCTGGCCAAATGAGCTTCC |
| of B2M target | | target site-F8 | |
| site | | | |
| DNA sequence | 4738 | MG29-1-B2M- | GAAGAATAAACCGTGACTTGGT |
| of B2M target | | target site-G8 | |
| site | | | |
| DNA sequence | 4739 | MG29-1-B2M- | AAGAATAAACCGTGACTTGGTA |
| of B2M target | | target site-H8 | |
| site | | | |
| DNA sequence | 4740 | MG29-1-B2M- | CCTCATAATTCCTCTATACATG |
| of B2M target | | target site-A9 | |
| site | | | |
| DNA sequence | 4741 | MG29-1-B2M- | TTGTTTTTTTTCTAGCAGATTT |
| of B2M target | | target site-B9 | |
| site | | | |
| DNA sequence | 4742 | MG29-1-B2M- | TGTTTTTTTTCTAGCAGATTTC |
| of B2M target | | target site-C9 | |
| site | | | |
| DNA sequence | 4743 | MG29-1-B2M- | GTTTTTTTTCTAGCAGATTTCT |
| of B2M target | | target site-D9 | |
| site | | | |
| DNA sequence | 4744 | MG29-1-B2M- | TTTTTTTTCTAGCAGATTTCTA |
| of B2M target | | target site-E9 | |
| site | | | |
| DNA sequence | 4745 | MG29-1-B2M- | TTTTCTAGCAGATTTCTAGCAG |
| of B2M target | | target site-F9 | |
| site | | | |
| DNA sequence | 4746 | MG29-1-B2M- | TTTCTAGCAGATTTCTAGCAGT |
| of B2M target | | target site-G9 | |
| site | | | |
| DNA sequence | 4747 | MG29-1-B2M- | TTCTAGCAGATTTCTAGCAGTA |
| of B2M target | | target site-H9 | |
| site | | | |
| DNA sequence | 4748 | MG29-1-B2M- | TCTAGCAGATTTCTAGCAGTAT |
| of B2M target | | target site-A10 | |
| site | | | |
| DNA sequence | 4749 | MG29-1-B2M- | CTAGCAGATTTCTAGCAGTATC |
| of B2M target | | target site-B10 | |
| site | | | |
| DNA sequence | 4750 | MG29-1-B2M- | TAGCAGATTTCTAGCAGTATCT |
| of B2M target | | target site-C10 | |
| site | | | |
| DNA sequence | 4751 | MG29-1-B2M- | TAGCAGTATCTTCTGTCACTGG |
| of B2M target | | target site-D10 | |
| site | | | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 16—Gene Editing Outcomes at the DNA Level for CD2

Primary T cells were purified from PMBCs using a negative selection kit (Miltenyi) according to the manufacturer's recommendations. Nucleofection of MG29-1 RNPs (126 pmol protein/160 pmol guide) (SEQ ID NOs: 4752-4836) was performed into T cells (200,000) using the Lonza 4D electroporator. Cells were harvested and genomic DNA prepared five days post-transfection. PCR primers appropriate for use in NGS-based DNA sequencing were generated, optimized, and used to amplify the individual target sequences for each guide RNA (SEQ ID NOs: 4837-4921). The amplicons were sequenced on an Illumina MiSeq machine and analyzed with a proprietary Python script to measure gene editing (FIG. 62).

TABLE 14E
Sequences of Guide RNAs and Sequences Targeted for Example 42

| | SEQ | | |
|---|---|---|---|
| | ID | | |
| Guide Target | NO | Guide Name | SEQUENCE |
| MG29-1 | 4752 | MG29-1-CD2- | /AltR1/rUrArArUrUrUrCrUrArCrUrGrUrUrGrUrArGrA |
| sgRNA | | sgRNA-A1 | rUrCrArUrGrUrArArArUrUrUrGrUrArGrCrCrArGrCr |
| targeting CD2 | | | UrU/AltR2/ |
| MG29-1 | 4753 | MG29-1-CD2- | /AltR1/rUrArArUrUrUrCrUrArCrUrGrUrUrGrUrArGrA |
| sgRNA | | sgRNA-B1 | rUrUrArGrCrCrArGrCrUrUrCrCrUrUrCrUrGrArUrUr |
| targeting CD2 | | | UrU/AltR2/ |
| MG29-1 | 4754 | MG29-1-CD2- | /AltR1/rUrArArUrUrUrCrUrArCrUrGrUrUrGrUrArGrA |
| sgRNA | | sgRNA-C1 | rUrArCrUrCrUrUrArUrGrCrUrUrArCrCrUrUrUrGrGr |
| targeting CD2 | | | ArA/AltR2/ |
| MG29-1 | 4755 | MG29-1-CD2- | /AltR1/rUrArArUrUrUrCrUrArCrUrGrUrUrGrUrArGrA |
| sgRNA | | sgRNA-D1 | rUrGrArArGrArArArCrArUrUrGrArArArArUrCrArGr |
| targeting CD2 | | | ArA/AltR2/ |
| MG29-1 | 4756 | MG29-1-CD2- | /AltR1/rUrArArUrUrUrCrUrArCrUrGrUrUrGrUrArGrA |
| sgRNA | | sgRNA-E1 | rUrGrCrUrUrUrUrUrArUrArGrGrUrGrCrArGrUrCrUr |
| targeting CD2 | | | CrC/AltR2/ |
| MG29-1 | 4757 | MG29-1-CD2- | /AltR1/rUrArArUrUrUrCrUrArCrUrGrUrUrGrUrArGrA |
| sgRNA | | sgRNA-F1 | rUrCrUrUrUrUrUrArUrArGrGrUrGrCrArGrUrCrUrCr |
| targeting CD2 | | | CrA/AltR2/ |
| MG29-1 | 4758 | MG29-1-CD2- | /AltR1/rUrArArUrUrUrCrUrArCrUrGrUrUrGrUrArGrA |
| sgRNA | | sgRNA-G1 | rUrUrArUrArGrGrUrGrCrArGrUrCrUrCrCrArArArGr |
| targeting CD2 | | | ArG/AltR2/ |
| MG29-1 | 4759 | MG29-1-CD2- | /AltR1/rUrArArUrUrUrCrUrArCrUrGrUrUrGrUrArGrA |
| sgRNA | | sgRNA-H1 | rUrArUrArGrGrUrGrCrArGrUrCrUrCrCrArArArGrAr |
| targeting CD2 | | | GrA/AltR2/ |
| MG29-1 | 4760 | MG29-1-CD2- | /AltR1/rUrArArUrUrUrCrUrArCrUrGrUrUrGrUrArGrA |
| sgRNA | | sgRNA-A2 | rUrUrArGrGrUrGrCrArGrUrCrUrCrCrArArArGrArGr |
| targeting CD2 | | | ArU/AltR2/ |
| MG29-1 | 4761 | MG29-1-CD2- | /AltR1/rUrArArUrUrUrCrUrArCrUrGrUrUrGrUrArGrA |
| sgRNA | | sgRNA-B2 | rUrCrArArArUrGrArGrUrGrArUrGrArUrArUrUrGrAr |
| targeting CD2 | | | CrG/AltR2/ |
| MG29-1 | 4762 | MG29-1-CD2- | /AltR1/rUrArArUrUrUrCrUrArCrUrGrUrUrGrUrArGrA |
| sgRNA | | sgRNA-C2 | rUrArArArUrGrArGrUrGrArUrGrArUrArUrUrGrArCr |
| targeting CD2 | | | GrA/AltR2/ |
| MG29-1 | 4763 | MG29-1-CD2- | /AltR1/rUrArArUrUrUrCrUrArCrUrGrUrUrGrUrArGrA |
| sgRNA | | sgRNA-D2 | rUrArArGrGrArArArArArGrArUrArCrArUrArUrArAr |
| targeting CD2 | | | GrC/AltR2/ |
| MG29-1 | 4764 | MG29-1-CD2- | /AltR1/rUrArArUrUrUrCrUrArCrUrGrUrUrGrUrArGrA |
| sgRNA | | sgRNA-E2 | rUrArArArArUrGrGrArArCrUrCrUrGrArArArArUrUr |
| targeting CD2 | | | ArA/AltR2/ |
| MG29-1 | 4765 | MG29-1-CD2- | /AltR1/rUrArArUrUrUrCrUrArCrUrGrUrUrGrUrArGrA |
| sgRNA | | sgRNA-F2 | rUrUrUrCrCrArArCrArCrArUrUrUrUrUrUrCrCrUrUr |
| targeting CD2 | | | UrU/AltR2/ |
| MG29-1 | 4766 | MG29-1-CD2- | /AltR1/rUrArArUrUrUrCrUrArCrUrGrUrUrGrUrArGrA |
| sgRNA | | sgRNA-G2 | rUrUrCrCrArArCrArCrArUrUrUrUrUrUrCrCrUrUrUr |
| targeting CD2 | | | UrG/AltR2/ |
| MG29-1 | 4767 | MG29-1-CD2- | /AltR1/rUrArArUrUrUrCrUrArCrUrGrUrUrGrUrArGrA |
| sgRNA | | sgRNA-H2 | rUrCrCrArArCrArCrArUrUrUrUrUrUrCrCrUrUrUrUr |
| targeting CD2 | | | GrU/AltR2/ |
| MG29-1 | 4768 | MG29-1-CD2- | /AltR1/rUrArArUrUrUrCrUrArCrUrGrUrUrGrUrArGrA |
| sgRNA | | sgRNA-A3 | rUrCrArArCrArCrArUrUrUrUrUrUrCrCrUrUrUrUrGr |
| targeting CD2 | | | UrA/AltR2/ |
| MG29-1 | 4769 | MG29-1-CD2- | /AltR1/rUrArArUrUrUrCrUrArCrUrGrUrUrGrUrArGrA |
| sgRNA | | sgRNA-B3 | rUrUrUrCrCrUrUrUrUrGrUrArUrCrArUrArUrArUrUr |
| targeting CD2 | | | GrA/AltR2/ |
| MG29-1 | 4770 | MG29-1-CD2- | /AltR1/rUrArArUrUrUrCrUrArCrUrGrUrUrGrUrArGrA |
| sgRNA | | sgRNA-C3 | rUrUrCrCrUrUrUrUrGrUrArUrCrArUrArUrArUrUrGr |
| targeting CD2 | | | ArU/AltR2/ |
| MG29-1 | 4771 | MG29-1-CD2- | /AltR1/rUrArArUrUrUrCrUrArCrUrGrUrUrGrUrArGrA |
| sgRNA | | sgRNA-D3 | rUrCrCrUrUrUrUrGrUrArUrCrArUrArUrArUrUrGrAr |
| targeting CD2 | | | UrA/AltR2/ |
| MG29-1 | 4772 | MG29-1-CD2- | /AltR1/rUrArArUrUrUrCrUrArCrUrGrUrUrGrUrArGrA |
| sgRNA | | sgRNA-E3 | rUrCrUrUrUrUrGrUrArUrCrArUrArUrArUrUrGrArUr |
| targeting CD2 | | | ArC/AltR2/ |
| MG29-1 | 4773 | MG29-1-CD2- | /AltR1/rUrArArUrUrUrCrUrArCrUrGrUrUrGrUrArGrA |
| sgRNA | | sgRNA-F3 | rUrGrUrArUrCrArUrArUrArUrUrGrArUrArCrCrUrUr |
| targeting CD2 | | | GrU/AltR2/ |
| MG29-1 | 4774 | MG29-1-CD2- | /AltR1/rUrArArUrUrUrCrUrArCrUrGrUrUrGrUrArGrA |
| sgRNA | | sgRNA-G3 | rUrUrArUrCrArUrArUrArUrUrGrArUrArCrCrUrUrGr |
| targeting CD2 | | | UrA/AltR2/ |
| MG29-1 | 4775 | MG29-1-CD2- | /AltR1/rUrArArUrUrUrCrUrArCrUrGrUrUrGrUrArGrA |
| sgRNA | | sgRNA-H3 | rUrCrArGrArGrUrUrCrCrArUrUrUrUrUrArArArUrAr |
| targeting CD2 | | | GrC/AltR2/ |
| MG29-1 | 4776 | MG29-1-CD2- | /AltR1/rUrArArUrUrUrCrUrArCrUrGrUrUrGrUrArGrA |
| sgRNA | | sgRNA-A4 | rUrArGrArGrUrUrCrCrArUrUrUrUrUrArArArUrArGr |
| targeting CD2 | | | CrU/AltR2/ |
| MG29-1 | 4777 | MG29-1-CD2- | /AltR1/rUrArArUrUrUrCrUrArCrUrGrUrUrGrUrArGrA |
| sgRNA | | sgRNA-B4 | rUrUrArArArUrArGrCrUrUrArUrArUrGrUrArUrCrUr |
| targeting CD2 | | | UrU/AltR2/ |
| MG29-1 | 4778 | MG29-1-CD2- | /AltR1/rUrArArUrUrUrCrUrArCrUrGrUrUrGrUrArGrA |
| sgRNA | | sgRNA-C4 | rUrArArArUrArGrCrUrUrArUrArUrGrUrArUrCrUrUr |
| targeting CD2 | | | UrU/AltR2/ |
| MG29-1 | 4779 | MG29-1-CD2- | /AltR1/rUrArArUrUrUrCrUrArCrUrGrUrUrGrUrArGrA |
| sgRNA | | sgRNA-D4 | rUrArArUrArGrCrUrUrArUrArUrGrUrArUrCrUrUrUr |
| targeting CD2 | | | UrU/AltR2/ |
| MG29-1 | 4780 | MG29-1-CD2- | /AltR1/rUrArArUrUrUrCrUrArCrUrGrUrUrGrUrArGrA |
| sgRNA | | sgRNA-E4 | rUrUrCrCrUrUrGrArArArGrUrCrUrCrUrUrUrCrUrCr |
| targeting CD2 | | | UrU/AltR2/ |
| MG29-1 | 4781 | MG29-1-CD2- | /AltR1/rUrArArUrUrUrCrUrArCrUrGrUrUrGrUrArGrA |
| sgRNA | | sgRNA-F4 | rUrCrCrUrUrGrArArArGrUrCrUrCrUrUrUrCrUrCrUr |
| targeting CD2 | | | UrU/AltR2/ |
| MG29-1 | 4782 | MG29-1-CD2- | /AltR1/rUrArArUrUrUrCrUrArCrUrGrUrUrGrUrArGrA |
| sgRNA | | sgRNA-G4 | rUrCrUrUrGrArArArGrUrCrUrCrUrUrUrCrUrCrUrUr |
| targeting CD2 | | | UrU/AltR2/ |
| MG29-1 | 4783 | MG29-1-CD2- | /AltR1/rUrArArUrUrUrCrUrArCrUrGrUrUrGrUrArGrA |
| sgRNA | | sgRNA-H4 | rUrUrCrUrUrUrUrCrUrGrArArUrUrGrUrGrCrArArUr |
| targeting CD2 | | | CrU/AltR2/ |
| MG29-1 | 4784 | MG29-1-CD2- | /AltR1/rUrArArUrUrUrCrUrArCrUrGrUrUrGrUrArGrA |
| sgRNA | | sgRNA-A5 | rUrCrUrGrArArUrUrGrUrGrCrArArUrCrUrUrUrUrUr |
| targeting CD2 | | | CrU/AltR2/ |
| MG29-1 | 4785 | MG29-1-CD2- | /AltR1/rUrArArUrUrUrCrUrArCrUrGrUrUrGrUrArGrA |
| sgRNA | | sgRNA-B5 | rUrUrGrArArUrUrGrUrGrCrArArUrCrUrUrUrUrUrCr |
| targeting CD2 | | | UrU/AltR2/ |
| MG29-1 | 4786 | MG29-1-CD2- | /AltR1/rUrArArUrUrUrCrUrArCrUrGrUrUrGrUrArGrA |
| sgRNA | | sgRNA-C5 | rUrUrCrUrUrGrUrCrUrGrArArGrUrUrUrUrUrUrCrCr |
| targeting CD2 | | | CrA/AltR2/ |
| MG29-1 | 4787 | MG29-1-CD2- | /AltR1/rUrArArUrUrUrCrUrArCrUrGrUrUrGrUrArGrA |
| sgRNA | | sgRNA-D5 | rUrCrUrUrGrUrCrUrGrArArGrUrUrUrUrUrUrCrCrCr |
| targeting CD2 | | | ArU/AltR2/ |
| MG29-1 | 4788 | MG29-1-CD2- | /AltR1/rUrArArUrUrUrCrUrArCrUrGrUrUrGrUrArGrA |
| sgRNA | | sgRNA-E5 | rUrUrUrGrUrCrUrGrArArGrUrUrUrUrUrUrCrCrCrAr |
| targeting CD2 | | | UrU/AltR2/ |
| MG29-1 | 4789 | MG29-1-CD2- | /AltR1/rUrArArUrUrUrCrUrArCrUrGrUrUrGrUrArGrA |
| sgRNA | | sgRNA-F5 | rUrUrUrCrCrCrArUrUrUrUrArUrArUrCrGrUrCrArAr |
| targeting CD2 | | | UrA/AltR2/ |
| MG29-1 | 4790 | MG29-1-CD2- | /AltR1/rUrArArUrUrUrCrUrArCrUrGrUrUrGrUrArGrA |
| sgRNA | | sgRNA-G5 | rUrUrCrCrCrArUrUrUrUrArUrArUrCrGrUrCrArArUr |
| targeting CD2 | | | ArU/AltR2/ |
| MG29-1 | 4791 | MG29-1-CD2- | /AltR1/rUrArArUrUrUrCrUrArCrUrGrUrUrGrUrArGrA |
| sgRNA | | sgRNA-H5 | rUrCrCrCrArUrUrUrUrArUrArUrCrGrUrCrArArUrAr |
| targeting CD2 | | | UrC/AltR2/ |
| MG29-1 | 4792 | MG29-1-CD2- | /AltR1/rUrArArUrUrUrCrUrArCrUrGrUrUrGrUrArGrA |
| sgRNA | | sgRNA-A6 | rUrCrCrArUrUrUrUrArUrArUrCrGrUrCrArArUrArUr |
| targeting CD2 | | | CrA/AltR2/ |
| MG29-1 | 4793 | MG29-1-CD2- | /AltR1/rUrArArUrUrUrCrUrArCrUrGrUrUrGrUrArGrA |
| sgRNA | | sgRNA-B6 | rUrArUrArUrCrGrUrCrArArUrArUrCrArUrCrArCrUr |
| targeting CD2 | | | CrA/AltR2/ |
| MG29-1 | 4794 | MG29-1-CD2- | /AltR1/rUrArArUrUrUrCrUrArCrUrGrUrUrGrUrArGrA |
| sgRNA | | sgRNA-C6 | rUrUrArUrCrGrUrCrArArUrArUrCrArUrCrArCrUrCr |
| targeting CD2 | | | ArU/AltR2/ |
| MG29-1 | 4795 | MG29-1-CD2- | /AltR1/rUrArArUrUrUrCrUrArCrUrGrUrUrGrUrArGrA |
| sgRNA | | sgRNA-D6 | rUrArArArArCrUrArGrGrArArUrGrUrCrCrArArGrUr |
| targeting CD2 | | | UrG/AltR2/ |
| MG29-1 | 4796 | MG29-1-CD2- | /AltR1/rUrArArUrUrUrCrUrArCrUrGrUrUrGrUrArGrA |
| sgRNA | | sgRNA-E6 | rUrCrArArGrGrCrArUrUrCrGrUrArArUrCrUrCrUrUr |
| targeting CD2 | | | UrG/AltR2/ |
| MG29-1 | 4797 | MG29-1-CD2- | /AltR1/rUrArArUrUrUrCrUrArCrUrGrUrUrGrUrArGrA |
| sgRNA | | sgRNA-F6 | rUrCrUrUrUrCrUrUrUrUrUrArGrArGrArGrGrGrUrCr |
| targeting CD2 | | | UrC/AltR2/ |
| MG29-1 | 4798 | MG29-1-CD2- | /AltR1/rUrArArUrUrUrCrUrArCrUrGrUrUrGrUrArGrA |
| sgRNA | | sgRNA-G6 | rUrUrUrUrUrUrArGrArGrArGrGrGrUrCrUrCrArArAr |
| targeting CD2 | | | ArC/AltR2/ |
| MG29-1 | 4799 | MG29-1-CD2- | /AltR1/rUrArArUrUrUrCrUrArCrUrGrUrUrGrUrArGrA |
| sgRNA | | sgRNA-H6 | rUrUrArGrArGrArGrGrGrUrCrUrCrArArArArCrCrAr |
| targeting CD2 | | | ArA/AltR2/ |
| MG29-1 | 4800 | MG29-1-CD2- | /AltR1/rUrArArUrUrUrCrUrArCrUrGrUrUrGrUrArGrA |
| sgRNA | | sgRNA-A7 | rUrArGrArGrArGrGrGrUrCrUrCrArArArArCrCrArAr |
| targeting CD2 | | | ArG/AltR2/ |
| MG29-1 | 4801 | MG29-1-CD2- | /AltR1/rUrArArUrUrUrCrUrArCrUrGrUrUrGrUrArGrA |
| sgRNA | | sgRNA-B7 | rUrGrArGrArGrGrGrUrCrUrCrArArArArCrCrArArAr |
| targeting CD2 | | | GrA/AltR2/ |
| MG29-1 | 4802 | MG29-1-CD2- | /AltR1/rUrArArUrUrUrCrUrArCrUrGrUrUrGrUrArGrA |
| sgRNA | | sgRNA-C7 | rUrUrCrArGrArGrGrGrUrCrArUrCrArCrArCrArCrAr |
| targeting CD2 | | | ArG/AltR2/ |
| MG29-1 | 4803 | MG29-1-CD2- | /AltR1/rUrArArUrUrUrCrUrArCrUrGrUrUrGrUrArGrA |
| sgRNA | | sgRNA-D7 | rUrUrUrCrCrCrUrGrCrUrGrUrGrCrArCrUrUrGrArAr |
| targeting CD2 | | | UrU/AltR2/ |
| MG29-1 | 4804 | MG29-1-CD2- | /AltR1/rUrArArUrUrUrCrUrArCrUrGrUrUrGrUrArGrA |
| sgRNA | | sgRNA-E7 | rUrGrCrArCrUrCrArGrGrCrUrGrGrUrGrGrUrCrCrAr |
| targeting CD2 | | | CrU/AltR2/ |
| MG29-1 | 4805 | MG29-1-CD2- | /AltR1/rUrArArUrUrUrCrUrArCrUrGrUrUrGrUrArGrA |
| sgRNA | | sgRNA-F7 | rUrCrArCrUrCrArGrGrCrUrGrGrUrGrGrUrCrCrArCr |
| targeting CD2 | | | UrU/AltR2/ |
| MG29-1 | 4806 | MG29-1-CD2- | /AltR1/rUrArArUrUrUrCrUrArCrUrGrUrUrGrUrArGrA |
| sgRNA | | sgRNA-G7 | rUrArGrArUrGrUrUrUrCrCrCrArUrCrUrUrGrArUrAr |
| targeting CD2 | | | CrA/AltR2/ |
| MG29-1 | 4807 | MG29-1-CD2- | /AltR1/rUrArArUrUrUrCrUrArCrUrGrUrUrGrUrArGrA |
| sgRNA | | sgRNA-H7 | rUrGrArUrGrUrUrUrCrCrCrArUrCrUrUrGrArUrArCr |
| targeting CD2 | | | ArG/AltR2/ |
| MG29-1 | 4808 | MG29-1-CD2- | /AltR1/rUrArArUrUrUrCrUrArCrUrGrUrUrGrUrArGrA |
| sgRNA | | sgRNA-A8 | rUrCrCrArUrCrUrUrGrArUrArCrArGrGrUrUrUrArAr |
| targeting CD2 | | | UrU/AltR2/ |
| MG29-1 | 4809 | MG29-1-CD2- | /AltR1/rUrArArUrUrUrCrUrArCrUrGrUrUrGrUrArGrA |
| sgRNA | | sgRNA-B8 | rUrArUrUrCrGrGrGrGrUrCrArGrUrUrCrCrArUrUrCr |
| targeting CD2 | | | ArU/AltR2/ |
| MG29-1 | 4810 | MG29-1-CD2- | /AltR1/rUrArArUrUrUrCrUrArCrUrGrUrUrGrUrArGrA |
| sgRNA | | sgRNA-C8 | rUrGrCrArGrArGrArArArGrGrUrCrUrGrGrArCrArUr |
| targeting CD2 | | | CrU/AltR2/ |
| MG29-1 | 4811 | MG29-1-CD2- | /AltR1/rUrArArUrUrUrCrUrArCrUrGrUrUrGrUrArGrA |
| sgRNA | | sgRNA-D8 | rUrCrArGrArGrArArArGrGrUrCrUrGrGrArCrArUrCr |
| targeting CD2 | | | UrA/AltR2/ |
| MG29-1 | 4812 | MG29-1-CD2- | /AltR1/rUrArArUrUrUrCrUrArCrUrGrUrUrGrUrArGrA |
| sgRNA | | sgRNA-E8 | rUrUrGrGrCrArCrUrGrCrUrCrGrUrUrUrUrCrUrArUr |
| targeting CD2 | | | ArU/AltR2/ |
| MG29-1 | 4813 | MG29-1-CD2- | /AltR1/rUrArArUrUrUrCrUrArCrUrGrUrUrGrUrArGrA |
| sgRNA | | sgRNA-F8 | rUrCrUrArUrArUrCrArCrCrArArArArGrGrArArArAr |
| targeting CD2 | | | ArA/AltR2/ |
| MG29-1 | 4814 | MG29-1-CD2- | /AltR1/rUrArArUrUrUrCrUrArCrUrGrUrUrGrUrArGrA |
| sgRNA | | sgRNA-G8 | rUrUrArUrArUrCrArCrCrArArArArGrGrArArArArAr |
| targeting CD2 | | | ArC/AltR2/ |
| MG29-1 | 4815 | MG29-1-CD2- | /AltR1/rUrArArUrUrUrCrUrArCrUrGrUrUrGrUrArGrA |
| sgRNA | | sgRNA-H8 | rUrUrCrCrGrArCrUrCrCrUrCrUrGrUrUrUrUrUrUrCr |
| targeting CD2 | | | CrU/AltR2/ |
| MG29-1 | 4816 | MG29-1-CD2- | /AltR1/rUrArArUrUrUrCrUrArCrUrGrUrUrGrUrArGrA |
| sgRNA | | sgRNA-A9 | rUrUrUrCrCrUrUrUrUrGrGrUrGrArUrArUrArGrArAr |
| targeting CD2 | | | ArA/AltR2/ |
| MG29-1 | 4817 | MG29-1-CD2- | /AltR1/rUrArArUrUrUrCrUrArCrUrGrUrUrGrUrArGrA |
| sgRNA | | sgRNA-B9 | rUrUrCrCrUrUrUrUrGrGrUrGrArUrArUrArGrArArAr |
| targeting CD2 | | | ArC/AltR2/ |
| MG29-1 | 4818 | MG29-1-CD2- | /AltR1/rUrArArUrUrUrCrUrArCrUrGrUrUrGrUrArGrA |
| sgRNA | | sgRNA-C9 | rUrCrCrUrUrUrUrGrGrUrGrArUrArUrArGrArArArAr |
| targeting CD2 | | | CrG/AltR2/ |
| MG29-1 | 4819 | MG29-1-CD2- | /AltR1/rUrArArUrUrUrCrUrArCrUrGrUrUrGrUrArGrA |
| sgRNA | | sgRNA-D9 | rUrCrUrUrUrUrGrGrUrGrArUrArUrArGrArArArArCr |
| targeting CD2 | | | GrA/AltR2/ |
| MG29-1 | 4820 | MG29-1-CD2- | /AltR1/rUrArArUrUrUrCrUrArCrUrGrUrUrGrUrArGrA |
| sgRNA | | sgRNA-E9 | rUrGrGrUrGrArUrArUrArGrArArArArCrGrArGrCrAr |
| targeting CD2 | | | GrU/AltR2/ |
| MG29-1 | 4821 | MG29-1-CD2- | /AltR1/rUrArArUrUrUrCrUrArCrUrGrUrUrGrUrArGrA |
| sgRNA | | sgRNA-F9 | rUrGrUrGrArUrArUrArGrArArArArCrGrArGrCrArGr |
| targeting CD2 | | | UrG/AltR2/ |
| MG29-1 | 4822 | MG29-1-CD2- | /AltR1/rUrArArUrUrUrCrUrArCrUrGrUrUrGrUrArGrA |
| sgRNA | | sgRNA-G9 | rUrUrCrUrGrCrArArArArGrGrArArGrArGrArArGrUr |
| targeting CD2 | | | GrG/AltR2/ |
| MG29-1 | 4823 | MG29-1-CD2- | /AltR1/rUrArArUrUrUrCrUrArCrUrGrUrUrGrUrArGrA |
| sgRNA | | sgRNA-H9 | rUrGrUrUrGrUrUrGrCrArGrArUrGrArGrGrArGrCrUr |
| targeting CD2 | | | GrG/AltR2/ |
| MG29-1 | 4824 | MG29-1-CD2- | /AltR1/rUrArArUrUrUrCrUrArCrUrGrUrUrGrUrArGrA |
| sgRNA | | sgRNA-A10 | rUrUrUrGrUrUrGrCrArGrArUrGrArGrGrArGrCrUrGr |
| targeting CD2 | | | GrA/AltR2/ |
| MG29-1 | 4825 | MG29-1-CD2- | /AltR1/rUrArArUrUrUrCrUrArCrUrGrUrUrGrUrArGrA |
| sgRNA | | sgRNA-B10 | rUrUrArUrCrUrUrUrUrUrUrArArUrUrArGrArGrGrAr |
| targeting CD2 | | | ArG/AltR2/ |
| MG29-1 | 4826 | MG29-1-CD2- | /AltR1/rUrArArUrUrUrCrUrArCrUrGrUrUrGrUrArGrA |
| sgRNA | | sgRNA-C10 | rUrUrUrArArUrUrArGrArGrGrArArGrGrGrGrArCrAr |
| targeting CD2 | | | ArU/AltR2/ |
| MG29-1 | 4827 | MG29-1-CD2- | /AltR1/rUrArArUrUrUrCrUrArCrUrGrUrUrGrUrArGrA |
| sgRNA | | sgRNA-D10 | rUrUrArArUrUrArGrArGrGrArArGrGrGrGrArCrArAr |
| targeting CD2 | | | UrG/AltR2/ |
| MG29-1 | 4828 | MG29-1-CD2- | /AltR1/rUrArArUrUrUrCrUrArCrUrGrUrUrGrUrArGrA |
| sgRNA | | sgRNA-E10 | rUrArArUrUrArGrArGrGrArArGrGrGrGrArCrArArUr |
| targeting CD2 | | | GrA/AltR2/ |
| MG29-1 | 4829 | MG29-1-CD2- | /AltR1/rUrArArUrUrUrCrUrArCrUrGrUrUrGrUrArGrA |
| sgRNA | | sgRNA-F10 | rUrArUrUrArGrArGrGrArArGrGrGrGrArCrArArUrGr |
| targeting CD2 | | | ArG/AltR2/ |
| MG29-1 | 4830 | MG29-1-CD2- | /AltR1/rUrArArUrUrUrCrUrArCrUrGrUrUrGrUrArGrA |
| sgRNA | | sgRNA-G10 | rUrCrUrGrCrUrGrCrCrCrCrArUrGrGrGrGrArGrGrUr |
| targeting CD2 | | | UrU/AltR2/ |
| MG29-1 | 4831 | MG29-1-CD2- | /AltR1/rUrArArUrUrUrCrUrArCrUrGrUrUrGrUrArGrA |
| sgRNA | | sgRNA-H10 | rUrUrGrCrUrGrCrCrCrCrArUrGrGrGrGrArGrGrUrUr |
| targeting CD2 | | | UrU/AltR2/ |
| MG29-1 | 4832 | MG29-1-CD2- | /AltR1/rUrArArUrUrUrCrUrArCrUrGrUrUrGrUrArGrA |
| sgRNA | | sgRNA-A11 | rUrGrGrCrUrGrArArCrUrCrGrArGrGrUrCrUrGrGrGr |
| targeting CD2 | | | GrA/AltR2/ |
| MG29-1 | 4833 | MG29-1-CD2- | /AltR1/rUrArArUrUrUrCrUrArCrUrGrUrUrGrUrArGrA |
| sgRNA | | sgRNA-B11 | rUrGrCrUrGrArArCrUrCrGrArGrGrUrCrUrGrGrGrGr |
| targeting CD2 | | | ArG/AltR2/ |
| MG29-1 | 4834 | MG29-1-CD2- | /AltR1/rUrArArUrUrUrCrUrArCrUrGrUrUrGrUrArGrA |
| sgRNA | | sgRNA-C11 | rUrUrGrCrUrGrGrUrGrArArCrUrUrGrUrGrUrGrCrCr |
| targeting CD2 | | | CrG/AltR2/ |
| MG29-1 | 4835 | MG29-1-CD2- | /AltR1/rUrArArUrUrUrCrUrArCrUrGrUrUrGrUrArGrA |
| sgRNA | | sgRNA-D11 | rUrGrUrGrGrGrGrCrUrUrCrCrGrGrCrCrCrCrUrUrUr |
| targeting CD2 | | | CrU/AltR2/ |
| MG29-1 | 4836 | MG29-1-CD2- | /AltR1/rUrArArUrUrUrCrUrArCrUrGrUrUrGrUrArGrA |
| sgRNA | | sgRNA-E11 | rUrUrUrCrArGrUrArGrCrUrArCrUrCrUrGrUrGrGrGr |
| targeting CD2 | | | CrU/AltR2/ |
| DNA sequence | 4837 | MG29-1-CD2- | CATGTAAATTTGTAGCCAGCTT |
| of CD2 target | | target site-A1 | |
| site | | | |
| DNA sequence | 4838 | MG29-1-CD2- | TAGCCAGCTTCCTTCTGATTTT |
| of CD2 target | | target site-B1 | |
| site | | | |
| DNA sequence | 4839 | MG29-1-CD2- | ACTCTTATGCTTACCTTTGGAA |
| of CD2 target | | target site-C1 | |
| site | | | |
| DNA sequence | 4840 | MG29-1-CD2- | GAAGAAACATTGAAAATCAGAA |
| of CD2 target | | target site-D1 | |
| site | | | |
| DNA sequence | 4841 | MG29-1-CD2- | GCTTTTTATAGGTGCAGTCTCC |
| of CD2 target | | target site-E1 | |
| site | | | |
| DNA sequence | 4842 | MG29-1-CD2- | CTTTTTATAGGTGCAGTCTCCA |
| of CD2 target | | target site-F1 | |
| site | | | |
| DNA sequence | 4843 | MG29-1-CD2- | TATAGGTGCAGTCTCCAAAGAG |
| of CD2 target | | target site-G1 | |
| site | | | |
| DNA sequence | 4844 | MG29-1-CD2- | ATAGGTGCAGTCTCCAAAGAGA |
| of CD2 target | | target site-H1 | |
| site | | | |
| DNA sequence | 4845 | MG29-1-CD2- | TAGGTGCAGTCTCCAAAGAGAT |
| of CD2 target | | target site-A2 | |
| site | | | |
| DNA sequence | 4846 | MG29-1-CD2- | CAAATGAGTGATGATATTGACG |
| of CD2 target | | target site-B2 | |
| site | | | |
| DNA sequence | 4847 | MG29-1-CD2- | AAATGAGTGATGATATTGACGA |
| of CD2 target | | target site-C2 | |
| site | | | |
| DNA sequence | 4848 | MG29-1-CD2- | AAGGAAAAAGATACATATAAGC |
| of CD2 target | | target site-D2 | |
| site | | | |
| DNA sequence | 4849 | MG29-1-CD2- | AAAATGGAACTCTGAAAATTAA |
| of CD2 target | | target site-E2 | |
| site | | | |
| DNA sequence | 4850 | MG29-1-CD2- | TTCCAACACATTTTTTCCTTTT |
| of CD2 target | | target site-F2 | |
| site | | | |
| DNA sequence | 4851 | MG29-1-CD2- | TCCAACACATTTTTTCCTTTTG |
| of CD2 target | | target site-G2 | |
| site | | | |
| DNA sequence | 4852 | MG29-1-CD2- | CCAACACATTTTTTCCTTTTGT |
| of CD2 target | | target site-H2 | |
| site | | | |
| DNA sequence | 4853 | MG29-1-CD2- | CAACACATTTTTTCCTTTTGTA |
| of CD2 target | | target site-A3 | |
| site | | | |
| DNA sequence | 4854 | MG29-1-CD2- | TTCCTTTTGTATCATATATTGA |
| of CD2 target | | target site-B3 | |
| site | | | |
| DNA sequence | 4855 | MG29-1-CD2- | TCCTTTTGTATCATATATTGAT |
| of CD2 target | | target site-C3 | |
| site | | | |
| DNA sequence | 4856 | MG29-1-CD2- | CCTTTTGTATCATATATTGATA |
| of CD2 target | | target site-D3 | |
| site | | | |
| DNA sequence | 4857 | MG29-1-CD2- | CTTTTGTATCATATATTGATAC |
| of CD2 target | | target site-E3 | |
| site | | | |
| DNA sequence | 4858 | MG29-1-CD2- | GTATCATATATTGATACCTTGT |
| of CD2 target | | target site-F3 | |
| site | | | |
| DNA sequence | 4859 | MG29-1-CD2- | TATCATATATTGATACCTTGTA |
| of CD2 target | | target site-G3 | |
| site | | | |
| DNA sequence | 4860 | MG29-1-CD2- | CAGAGTTCCATTTTTAAATAGC |
| of CD2 target | | target site-H3 | |
| site | | | |
| DNA sequence | 4861 | MG29-1-CD2- | AGAGTTCCATTTTTAAATAGCT |
| of CD2 target | | target site-A4 | |
| site | | | |
| DNA sequence | 4862 | MG29-1-CD2- | TAAATAGCTTATATGTATCTTT |
| of CD2 target | | target site-B4 | |
| site | | | |
| DNA sequence | 4863 | MG29-1-CD2- | AAATAGCTTATATGTATCTTTT |
| of CD2 target | | target site-C4 | |
| site | | | |
| DNA sequence | 4864 | MG29-1-CD2- | AATAGCTTATATGTATCTTTTT |
| of CD2 target | | target site-D4 | |
| site | | | |
| DNA sequence | 4865 | MG29-1-CD2- | TCCTTGAAAGTCTCTTTCTCTT |
| of CD2 target | | target site-E4 | |
| site | | | |
| DNA sequence | 4866 | MG29-1-CD2- | CCTTGAAAGTCTCTTTCTCTTT |
| of CD2 target | | target site-F4 | |
| site | | | |
| DNA sequence | 4867 | MG29-1-CD2- | CTTGAAAGTCTCTTTCTCTTTT |
| of CD2 target | | target site-G4 | |
| site | | | |
| DNA sequence | 4868 | MG29-1-CD2- | TCTTTTCTGAATTGTGCAATCT |
| of CD2 target | | target site-H4 | |
| site | | | |
| DNA sequence | 4869 | MG29-1-CD2- | CTGAATTGTGCAATCTTTTTCT |
| of CD2 target | | target site-A5 | |
| site | | | |
| DNA sequence | 4870 | MG29-1-CD2- | TGAATTGTGCAATCTTTTTCTT |
| of CD2 target | | target site-B5 | |
| site | | | |
| DNA sequence | 4871 | MG29-1-CD2- | TCTTGTCTGAAGTTTTTTCCCA |
| of CD2 target | | target site-C5 | |
| site | | | |
| DNA sequence | 4872 | MG29-1-CD2- | CTTGTCTGAAGTTTTTTCCCAT |
| of CD2 target | | target site-D5 | |
| site | | | |
| DNA sequence | 4873 | MG29-1-CD2- | TTGTCTGAAGTTTTTTCCCATT |
| of CD2 target | | target site-E5 | |
| site | | | |
| DNA sequence | 4874 | MG29-1-CD2- | TTCCCATTTTATATCGTCAATA |
| of CD2 target | | target site-F5 | |
| site | | | |
| DNA sequence | 4875 | MG29-1-CD2- | TCCCATTTTATATCGTCAATAT |
| of CD2 target | | target site-G5 | |
| site | | | |
| DNA sequence | 4876 | MG29-1-CD2- | CCCATTTTATATCGTCAATATC |
| of CD2 target | | target site-H5 | |
| site | | | |
| DNA sequence | 4877 | MG29-1-CD2- | CCATTTTATATCGTCAATATCA |
| of CD2 target | | target site-A6 | |
| site | | | |
| DNA sequence | 4878 | MG29-1-CD2- | ATATCGTCAATATCATCACTCA |
| of CD2 target | | target site-B6 | |
| site | | | |
| DNA sequence | 4879 | MG29-1-CD2- | TATCGTCAATATCATCACTCAT |
| of CD2 target | | target site-C6 | |
| site | | | |
| DNA sequence | 4880 | MG29-1-CD2- | AAAACTAGGAATGTCCAAGTTG |
| of CD2 target | | target site-D6 | |
| site | | | |
| DNA sequence | 4881 | MG29-1-CD2- | CAAGGCATTCGTAATCTCTTTG |
| of CD2 target | | target site-E6 | |
| site | | | |
| DNA sequence | 4882 | MG29-1-CD2- | CTTTCTTTTTAGAGAGGGTCTC |
| of CD2 target | | target site-F6 | |
| site | | | |
| DNA sequence | 4883 | MG29-1-CD2- | TTTTTAGAGAGGGTCTCAAAAC |
| of CD2 target | | target site-G6 | |
| site | | | |
| DNA sequence | 4884 | MG29-1-CD2- | TAGAGAGGGTCTCAAAACCAAA |
| of CD2 target | | target site-H6 | |
| site | | | |
| DNA sequence | 4885 | MG29-1-CD2- | AGAGAGGGTCTCAAAACCAAAG |
| of CD2 target | | target site-A7 | |
| site | | | |
| DNA sequence | 4886 | MG29-1-CD2- | GAGAGGGTCTCAAAACCAAAGA |
| of CD2 target | | target site-B7 | |
| site | | | |
| DNA sequence | 4887 | MG29-1-CD2- | TCAGAGGGTCATCACACACAAG |
| of CD2 target | | target site-C7 | |
| site | | | |
| DNA sequence | 4888 | MG29-1-CD2- | TTCCCTGCTGTGCACTTGAATT |
| of CD2 target | | target site-D7 | |
| site | | | |
| DNA sequence | 4889 | MG29-1-CD2- | GCACTCAGGCTGGTGGTCCACT |
| of CD2 target | | target site-E7 | |
| site | | | |
| DNA sequence | 4890 | MG29-1-CD2- | CACTCAGGCTGGTGGTCCACTT |
| of CD2 target | | target site-F7 | |
| site | | | |
| DNA sequence | 4891 | MG29-1-CD2- | AGATGTTTCCCATCTTGATACA |
| of CD2 target | | target site-G7 | |
| site | | | |
| DNA sequence | 4892 | MG29-1-CD2- | GATGTTTCCCATCTTGATACAG |
| of CD2 target | | target site-H7 | |
| site | | | |
| DNA sequence | 4893 | MG29-1-CD2- | CCATCTTGATACAGGTTTAATT |
| of CD2 target | | target site-A8 | |
| site | | | |
| DNA sequence | 4894 | MG29-1-CD2- | ATTCGGGGTCAGTTCCATTCAT |
| of CD2 target | | target site-B8 | |
| site | | | |
| DNA sequence | 4895 | MG29-1-CD2- | CCATCTTGATACAGGTTTAATT |
| of CD2 target | | target site-C8 | |
| site | | | |
| DNA sequence | 4896 | MG29-1-CD2- | CAGAGAAAGGTCTGGACATCTA |
| of CD2 target | | target site-D8 | |
| site | | | |
| DNA sequence | 4897 | MG29-1-CD2- | TGGCACTGCTCGTTTTCTATAT |
| of CD2 target | | target site-E8 | |
| site | | | |
| DNA sequence | 4898 | MG29-1-CD2- | CTATATCACCAAAAGGAAAAAA |
| of CD2 target | | target site-F8 | |
| site | | | |
| DNA sequence | 4899 | MG29-1-CD2- | TATATCACCAAAAGGAAAAAAC |
| of CD2 target | | target site-G8 | |
| site | | | |
| DNA sequence | 4900 | MG29-1-CD2- | TCCGACTCCTCTGTTTTTTCCT |
| of CD2 target | | target site-H8 | |
| site | | | |
| DNA sequence | 4901 | MG29-1-CD2- | TTCCTTTTGGTGATATAGAAAA |
| of CD2 target | | target site-A9 | |
| site | | | |
| DNA sequence | 4902 | MG29-1-CD2- | TCCTTTTGGTGATATAGAAAAC |
| of CD2 target | | target site-B9 | |
| site | | | |
| DNA sequence | 4903 | MG29-1-CD2- | CCTTTTGGTGATATAGAAAACG |
| of CD2 target | | target site-C9 | |
| site | | | |
| DNA sequence | 4904 | MG29-1-CD2- | CTTTTGGTGATATAGAAAACGA |
| of CD2 target | | target site-D9 | |
| site | | | |
| DNA sequence | 4905 | MG29-1-CD2- | GGTGATATAGAAAACGAGCAGT |
| of CD2 target | | target site-E9 | |
| site | | | |
| DNA sequence | 4906 | MG29-1-CD2- | GTGATATAGAAAACGAGCAGTG |
| of CD2 target | | target site-F9 | |
| site | | | |
| DNA sequence | 4907 | MG29-1-CD2- | TCTGCAAAAGGAAGAGAAGTGG |
| of CD2 target | | target site-G9 | |
| site | | | |
| DNA sequence | 4908 | MG29-1-CD2- | GTTGTTGCAGATGAGGAGCTGG |
| of CD2 target | | target site-H9 | |
| site | | | |
| DNA sequence | 4909 | MG29-1-CD2- | TTGTTGCAGATGAGGAGCTGGA |
| of CD2 target | | target site-A10 | |
| site | | | |
| DNA sequence | 4910 | MG29-1-CD2- | TATCTTTTTTAATTAGAGGAAG |
| of CD2 target | | target site-B10 | |
| site | | | |
| DNA sequence | 4911 | MG29-1-CD2- | TTAATTAGAGGAAGGGGACAAT |
| of CD2 target | | target site-C10 | |
| site | | | |
| DNA sequence | 4912 | MG29-1-CD2- | TAATTAGAGGAAGGGGACAATG |
| of CD2 target | | target site-D10 | |
| site | | | |
| DNA sequence | 4913 | MG29-1-CD2- | AATTAGAGGAAGGGGACAATGA |
| of CD2 target | | target site-E10 | |
| site | | | |
| DNA sequence | 4914 | MG29-1-CD2- | ATTAGAGGAAGGGGACAATGAG |
| of CD2 target | | target site-F10 | |
| site | | | |
| DNA sequence | 4915 | MG29-1-CD2- | CTGCTGCCCCATGGGGAGGTTT |
| of CD2 target | | target site-G10 | |
| site | | | |
| DNA sequence | 4916 | MG29-1-CD2- | TGCTGCCCCATGGGGAGGTTTT |
| of CD2 target | | target site-H10 | |
| site | | | |
| DNA sequence | 4917 | MG29-1-CD2- | GGCTGAACTCGAGGTCTGGGGA |
| of CD2 target | | target site-A11 | |
| site | | | |
| DNA sequence | 4918 | MG29-1-CD2- | GCTGAACTCGAGGTCTGGGGAG |
| of CD2 target | | target site-B11 | |
| site | | | |
| DNA sequence | 4919 | MG29-1-CD2- | TGCTGGTGAACTTGTGTGCCCG |
| of CD2 target | | target site-C11 | |
| site | | | |
| DNA sequence | 4920 | MG29-1-CD2- | GTGGGGCTTCCGGCCCCTTTCT |
| of CD2 target | | target site-D11 | |
| site | | | |
| DNA sequence | 4921 | MG29-1-CD2- | TTCAGTAGCTACTCTGTGGGCT |
| of CD2 target | | target site-E11 | |
| site | | | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 17—Gene Editing Outcomes at the DNA Level for CD5

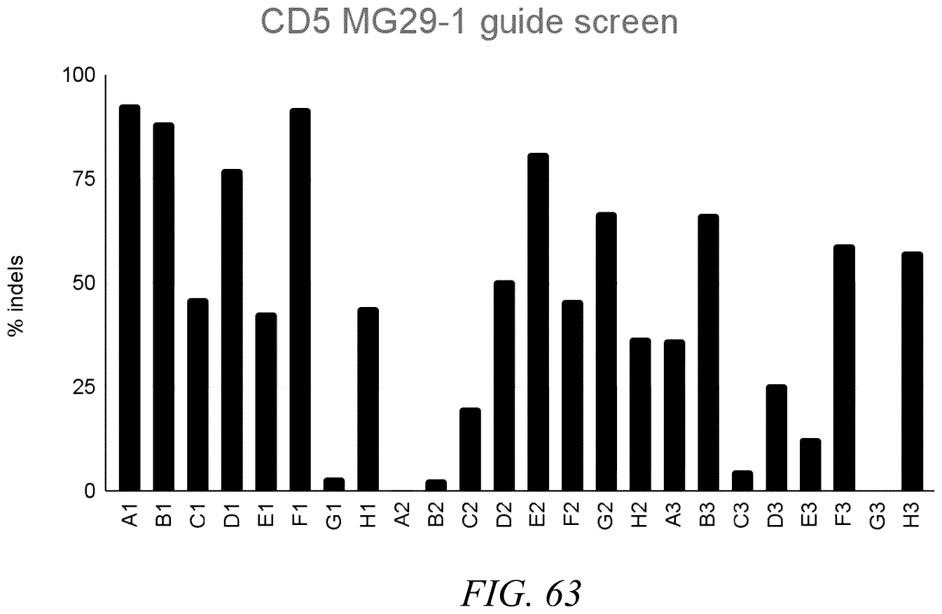

Primary T cells were purified from PMBCs using a negative selection kit (Miltenyi) according to the manufacturer's recommendations. Nucleofection of MG29-1 RNPs (126 pmol protein/160 pmol guide) (SEQ ID NOs: 4922-4945) was performed into T cells (200,000) using the Lonza 4D electroporator. Cells were harvested and genomic DNA prepared five days post-transfection. PCR primers appropriate for use in NGS-based DNA sequencing were generated, optimized, and used to amplify the individual target sequences for each guide RNA (SEQ ID NOs: 4946-4969). The amplicons were sequenced on an Illumina MiSeq machine and analyzed with a proprietary Python script to measure gene editing (FIG. 63).

TABLE 14F
Sequences of Guide RNAs and Sequences
Targeted for Example 43

| Guide | SEQ ID | | |
|---|---|---|---|
| Target | NO | Guide Name | SEQUENCE |
| MG29-1 | 4922 | MG29-1-CD5- | /AltR1/rUrArArUrUrUr |
| sgRNA | | sgRNA-A1 | CrUrArCrUrGrUrUrGrUr |
| targeting | | | ArGrArUrUrGrCrArGrUr |
| CD5 | | | CrGrCrUrUrCrCrUrGrCr |
| | | | CrUrCrGrGrA/AltR2/ |
| MG29-1 | 4923 | MG29-1-CD5- | /AltR1/rUrArArUrUrUr |
| sgRNA | | sgRNA-B1 | CrUrArCrUrGrUrUrGrUr |
| targeting | | | ArGrArUrUrCrCrArUrGr |
| CD5 | | | UrGrGrCrUrCrUrUrCrCr |
| | | | UrUrArCrCrU/AltR2/ |
| MG29-1 | 4924 | MG29-1-CD5- | /AltR1/rUrArArUrUrUr |
| sgRNA | | sgRNA-C1 | CrUrArCrUrGrUrUrGrUr |
| targeting | | | ArGrArUrUrGrArCrCrCr |
| CD5 | | | CrCrArGrArUrUrUrCrCr |
| | | | ArGrGrCrArA/AltR2/ |
| MG29-1 | 4925 | MG29-1-CD5- | /AltR1/rUrArArUrUrUr |
| sgRNA | | sgRNA-D1 | CrUrArCrUrGrUrUrGrUr |
| targeting | | | ArGrArUrCrArGrGrCrAr |
| CD5 | | | ArGrGrCrUrCrArCrCrCr |
| | | | GrUrUrCrCrA/AltR2/ |
| MG29-1 | 4926 | MG29-1-CD5- | /AltR1/rUrArArUrUrUr |
| sgRNA | | sgRNA-E1 | CrUrArCrUrGrUrUrGrUr |
| targeting | | | ArGrArUrCrArGrCrCrAr |
| CD5 | | | GrArGrCrUrGrGrGrGrCr |
| | | | CrGrGrArGrC/AltR2/ |
| MG29-1 | 4927 | MG29-1-CD5- | /AltR1/rUrArArUrUrUr |
| sgRNA | | sgRNA-F1 | CrUrArCrUrGrUrUrGrUr |
| targeting | | | ArGrArUrUrGrCrUrGrUr |
| CD5 | | | GrGrCrUrGrCrArGrUrUr |
| | | | GrGrArGrArA/AltR2/ |
| MG29-1 | 4928 | MG29-1-CD5- | /AltR1/rUrArArUrUrUr |
| sgRNA | | sgRNA-G1 | CrUrArCrUrGrUrUrGrUr |
| targeting | | | ArGrArUrGrArCrGrCrUr |
| CD5 | | | UrGrArCrUrGrGrGrGrUr |
| | | | CrCrUrCrCrC/AltR2/ |
| MG29-1 | 4929 | MG29-1-CD5- | /AltR1/rUrArArUrUrUr |
| sgRNA | | sgRNA-H1 | CrUrArCrUrGrUrUrGrUr |
| targeting | | | ArGrArUrArCrGrCrUrUr |
| CD5 | | | GrArCrUrGrGrGrGrUrCr |
| | | | CrUrCrCrCrA/AltR2/ |
| MG29-1 | 4930 | MG29-1-CD5- | /AltR1/rUrArArUrUrUr |
| sgRNA | | sgRNA-A2 | CrUrArCrUrGrUrUrGrUr |
| targeting | | | ArGrArUrCrUrGrGrGrGr |
| CD5 | | | CrUrUrGrArUrUrUrUrCr |
| | | | CrUrGrArArG/AltR2/ |
| MG29-1 | 4931 | MG29-1-CD5- | /AltR1/rUrArArUrUrUr |
| sgRNA | | sgRNA-B2 | CrUrArCrUrGrUrUrGrUr |
| targeting | | | ArGrArUrUrGrGrGrGrCr |
| CD5 | | | UrUrGrArUrUrUrUrCrCr |
| | | | UrGrArArGrC/AltR2/ |
| MG29-1 | 4932 | MG29-1-CD5- | /AltR1/rUrArArUrUrUr |
| sgRNA | | sgRNA-C2 | CrUrArCrUrGrUrUrGrUr |
| targeting | | | ArGrArUrCrCrUrGrArAr |
| CD5 | | | GrCrArArUrGrCrUrCrCr |
| | | | ArGrGrGrArG/AltR2/ |
| MG29-1 | 4933 | MG29-1-CD5- | /AltR1/rUrArArUrUrUr |
| sgRNA | | sgRNA-D2 | CrUrArCrUrGrUrUrGrUr |
| targeting | | | ArGrArUrCrUrGrArArGr |
| CD5 | | | CrArArUrGrCrUrCrCrAr |
| | | | GrGrGrArGrG/AltR2/ |
| MG29-1 | 4934 | MG29-1-CD5- | /AltR1/rUrArArUrUrUr |
| sgRNA | | sgRNA-E2 | CrUrArCrUrGrUrUrGrUr |
| targeting | | | ArGrArUrCrCrArUrUrGr |
| CD5 | | | CrUrUrCrCrCrCrUrCrUr |
| | | | CrArGrGrUrU/AltR2/ |
| MG29-1 | 4935 | MG29-1-CD5- | /AltR1/rUrArArUrUrUr |
| sgRNA | | sgRNA-F2 | CrUrArCrUrGrUrUrGrUr |
| targeting | | | ArGrArUrCrArGrCrCrCr |
| CD5 | | | ArArGrGrUrGrCrArGrAr |
| | | | GrCrCrGrUrC/AltR2/ |
| MG29-1 | 4936 | MG29-1-CD5- | /AltR1/rUrArArUrUrUr |
| sgRNA | | sgRNA-G2 | CrUrArCrUrGrUrUrGrUr |
| targeting | | | ArGrArUrGrGrArGrArGr |
| CD5 | | | ArArArUrUrCrCrUrArCr |
| | | | UrGrCrArArG/AltR2/ |
| MG29-1 | 4937 | MG29-1-CD5- | /AltR1/rUrArArUrUrUr |
| sgRNA | | sgRNA-H2 | CrUrArCrUrGrUrUrGrUr |
| targeting | | | ArGrArUrUrCrUrCrCrCr |
| CD5 | | | ArArArGrUrUrCrGrUrGr |
| | | | GrCrArCrUrG/AltR2/ |
| MG29-1 | 4938 | MG29-1-CD5- | /AltR1/rUrArArUrUrUr |
| sgRNA | | sgRNA-A3 | CrUrArCrUrGrUrUrGrUr |
| targeting | | | ArGrArUrUrUrCrArCrUr |
| CD5 | | | ArGrCrUrUrCrUrUrGrUr |
| | | | ArGrGrCrArA/AltR2/ |
| MG29-1 | 4939 | MG29-1-CD5- | /AltR1/rUrArArUrUrUr |
| sgRNA | | sgRNA-B3 | CrUrArCrUrGrUrUrGrUr |
| targeting | | | ArGrArUrUrUrGrUrCrUr |
| CD5 | | | CrUrUrGrCrCrCrArGrUr |
| | | | CrCrGrCrCrA/AltR2/ |
| MG29-1 | 4940 | MG29-1-CD5- | /AltR1/rUrArArUrUrUr |
| sgRNA | | sgRNA-C3 | CrUrArCrUrGrUrUrGrUr |
| targeting | | | ArGrArUrGrGrUrUrCrAr |
| CD5 | | | UrUrCrCrCrGrUrUrGrGr |
| | | | GrCrCrArArU/AltR2/ |
| MG29-1 | 4941 | MG29-1-CD5- | /AltR1/rUrArArUrUrUr |
| sgRNA | | sgRNA-D3 | CrUrArCrUrGrUrUrGrUr |
| targeting | | | ArGrArUrGrUrUrCrArUr |
| CD5 | | | UrCrCrCrGrUrUrGrGrGr |
| | | | CrCrArArUrC/AltR2/ |
| MG29-1 | 4942 | MG29-1-CD5- | /AltR1/rUrArArUrUrUr |
| sgRNA | | sgRNA-E3 | CrUrArCrUrGrUrUrGrUr |
| targeting | | | ArGrArUrCrArUrCrGrCr |
| CD5 | | | ArArCrCrArCrArCrGrGr |
| | | | CrArArCrCrG/AltR2/ |
| MG29-1 | 4943 | MG29-1-CD5- | /AltR1/rUrArArUrUrUr |
| sgRNA | | sgRNA-F3 | CrUrArCrUrGrUrUrGrUr |
| targeting | | | ArGrArUrUrUrUrCrCrCr |
| CD5 | | | CrArGrCrUrCrUrGrGrAr |
| | | | ArGrGrGrGrC/AltR2/ |
| MG29-1 | 4944 | MG29-1-CD5- | /AltR1/rUrArArUrUrUr |
| sgRNA | | sgRNA-G3 | CrUrArCrUrGrUrUrGrUr |
| targeting | | | ArGrArUrCrCrCrArGrCr |
| CD5 | | | UrCrUrGrGrArArGrGrGr |
| | | | GrCrUrCrUrG/AltR2/ |
| MG29-1 | 4945 | MG29-1-CD5- | /AltR1/rUrArArUrUrUr |
| sgRNA | | sgRNA-H3 | CrUrArCrUrGrUrUrGrUr |
| targeting | | | ArGrArUrCrArGrCrCrUr |
| CD5 | | | CrUrGrArGrCrCrCrCrAr |
| | | | UrGrCrArGrA/AltR2/ |
| DNA | 4946 | MG29-1-CD5- | TGCAGTCGCTTCCTGCCTCG |
| sequence | | target | GA |
| of CD5 | | site-A1 | |
| target | | | |
| site | | | |
| DNA | 4947 | MG29-1-CD5- | TCCATGTGGCTCTTCCTTAC |
| sequence | | target | CT |
| of CD5 | | site-B1 | |
| target | | | |
| site | | | |
| DNA | 4948 | MG29-1-CD5- | TGACCCCCAGATTTCCAGGC |
| sequence | | target | AA |
| of CD5 | | site-C1 | |
| target | | | |
| site | | | |
| DNA | 4949 | MG29-1-CD5- | CAGGCAAGGCTCACCCGTTC |
| sequence | | target | CA |
| of CD5 | | site-D1 | |
| target | | | |
| site | | | |
| DNA | 4950 | MG29-1-CD5- | CAGCCAGAGCTGGGGCCGGA |
| sequence | | target | GC |
| of CD5 | | site-E1 | |
| target | | | |
| site | | | |
| DNA | 4951 | MG29-1-CD5- | TGCTGTGGCTGCAGTTGGAG |
| sequence | | target | AA |
| of CD5 | | site-F1 | |
| target | | | |
| site | | | |
| DNA | 4952 | MG29-1-CD5- | GACGCTTGACTGGGGTCCTC |
| sequence | | target | CC |
| of CD5 | | site-G1 | |
| target | | | |
| site | | | |
| DNA | 4953 | MG29-1-CD5- | ACGCTTGACTGGGGTCCTCC |
| sequence | | target | CA |
| of CD5 | | site-H1 | |
| target | | | |
| site | | | |
| DNA | 4954 | MG29-1-CD5- | CTGGGGCTTGATTTTCCTGA |
| sequence | | target | AG |
| of CD5 | | site-A2 | |
| target | | | |
| site | | | |
| DNA | 4955 | MG29-1-CD5- | TGGGGCTTGATTTTCCTGAA |
| sequence | | target | GC |
| of CD5 | | site-B2 | |
| target | | | |
| site | | | |
| DNA | 4956 | MG29-1-CD5- | CCTGAAGCAATGCTCCAGGG |
| sequence | | target | AG |
| of CD5 | | site-C2 | |
| target | | | |
| site | | | |
| DNA | 4957 | MG29-1-CD5- | CTGAAGCAATGCTCCAGGGA |
| sequence | | target | GG |
| | | site-D2 | |
| of CD5 | | | |
| target | | | |
| site | | | |
| DNA | 4958 | MG29-1-CD5- | CCATTGCTTCCCCTCTCAGG |
| sequence | | target | TT |
| of CD5 | | site-E2 | |
| target | | | |
| site | | | |
| DNA | 4959 | MG29-1-CD5- | CAGCCCAAGGTGCAGAGCCG |
| sequence | | target | TC |
| of CD5 | | site-F2 | |
| target | | | |
| site | | | |
| DNA | 4960 | MG29-1-CD5- | GGAGAGAAATTCCTACTGCA |
| sequence | | target | AG |
| of CD5 | | site-G2 | |
| target | | | |
| site | | | |
| DNA | 4961 | MG29-1-CD5- | TCTCCCAAAGTTCGTGGCAC |
| sequence | | target | TG |
| of CD5 | | site-H2 | |
| target | | | |
| site | | | |
| DNA | 4962 | MG29-1-CD5- | TTCACTAGCTTCTTGTAGGC |
| sequence | | target | AA |
| of CD5 | | site-A3 | |
| target | | | |
| site | | | |
| DNA | 4963 | MG29-1-CD5- | TTGTCTCTTGCCCAGTCCGC |
| sequence | | target | CA |
| of CD5 | | site-B3 | |
| target | | | |
| site | | | |
| DNA | 4964 | MG29-1-CD5- | GGTTCATTCCCGTTGGGCCA |
| sequence | | target | AT |
| of CD5 | | site-C3 | |
| target | | | |
| site | | | |
| DNA | 4965 | MG29-1-CD5- | GTTCATTCCCGTTGGGCCAA |
| sequence | | target | TC |
| of CD5 | | site-D3 | |
| target | | | |
| site | | | |
| DNA | 4966 | MG29-1-CD5- | CATCGCAACCACACGGCAAC |
| sequence | | target | CG |
| of CD5 | | site-E3 | |
| target | | | |
| site | | | |
| DNA | 4967 | MG29-1-CD5- | TTTCCCCAGCTCTGGAAGGG |
| sequence | | target | GC |
| of CD5 | | site-F3 | |
| target | | | |
| site | | | |
| DNA | 4968 | MG29-1-CD5- | CCCAGCTCTGGAAGGGGCTC |
| sequence | | target | TG |
| of CD5 | | site-G3 | |
| target | | | |
| site | | | |
| DNA | 4969 | MG29-1-CD5- | CAGCCTCTGAGCCCCATGCA |
| sequence | | target | GA |
| of CD5 | | site-H3 | |
| target | | | |
| site | | | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 18—Gene Editing Outcomes at the DNA Level for Mouse TRAC

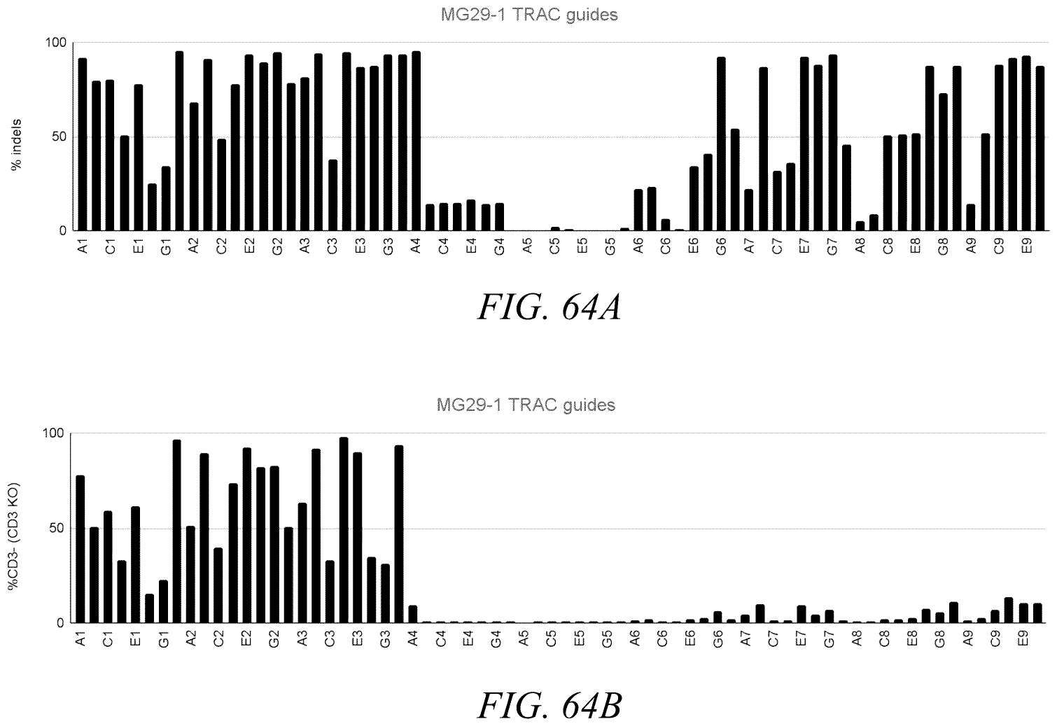

Primary T cells were purified from C57BL/6 mouse spleens. Nucleofection of MG29-1 RNPs (126 pmol protein/160 pmol guide) (SEQ ID NOs: 5056-5125) was performed into T cells (200,000) using the Lonza 4D electroporator and 100 pmol transfection enhancer (IDT). Cells were harvested and genomic DNA prepared five days post-transfection. PCR primers appropriate for use in NGS-based DNA sequencing were generated, optimized, and used to amplify the individual target sequences for each guide RNA (SEQ ID NOs: 5126-5195). The amplicons were sequenced on an Illumina MiSeq machine and analyzed with a proprietary Python script to measure gene editing (FIG. 64A).

TABLE 14G
Sequences of Guide RNAs and Sequences Targeted for Example 44

| | SEQ | Guide | |
|---|---|---|---|
| Guide | ID | | |
| Target | NO | Name | SEQUENCE |
| MG29-1 | 5056 | MG29-1- | /AltR1/rUrArArUrUrUr |
| sgRNA | | mTRAC- | CrUrArCrUrGrUrUrGrUr |
| targeting | | sgRNA-A1 | ArGrArUrArCrUrCrCrCr |
| mouse TRAC | | | ArArArUrCrArArUrGrUr |
| | | | GrCrCrGrArA/AltR2/ |
| MG29-1 | 5057 | MG29-1- | /AltR1/rUrArArUrUrUr |
| sgRNA | | mTRAC- | CrUrArCrUrGrUrUrGrUr |
| targeting | | sgRNA-B1 | ArGrArUrArArGrArUrAr |
| mouse TRAC | | | UrCrUrUrGrGrCrArGrGr |
| | | | UrGrArArGrC/AltR2/ |
| MG29-1 | 5058 | MG29-1- | /AltR1/rUrArArUrUrUr |
| sgRNA | | mTRAC- | CrUrArCrUrGrUrUrGrUr |
| targeting | | sgRNA-C1 | ArGrArUrArUrGrUrCrCr |
| mouse TRAC | | | ArGrCrArCrArGrUrUrUr |
| | | | UrGrUrCrArG/AltR2/ |
| MG29-1 | 5059 | MG29-1- | /AltR1/rUrArArUrUrUr |
| sgRNA | | mTRAC- | CrUrArCrUrGrUrUrGrUr |
| targeting | | sgRNA-D1 | ArGrArUrGrUrCrArGrUr |
| mouse TRAC | | | GrArUrGrArArCrGrUrUr |
| | | | CrCrArGrArU/AltR2 |
| MG29-1 | 5060 | MG29-1- | /AltR1/rUrArArUrUrUr |
| sgRNA | | mTRAC- | CrUrArCrUrGrUrUrGrUr |
| targeting | | sgRNA-E1 | ArGrArUrUrCrArGrUrGr |
| mouse TRAC | | | ArUrGrArArCrGrUrUrCr |
| | | | CrArGrArUrU/AltR2/ |
| MG29-1 | 5061 | MG29-1- | /AltR1/rUrArArUrUrUr |
| sgRNA | | mTRAC- | CrUrArCrUrGrUrUrGrUr |
| targeting | | sgRNA-F1 | ArGrArUrCrGrGrCrArCr |
| mouse TRAC | | | ArUrUrGrArUrUrUrGrGr |
| | | | GrArGrUrCrA/AItR2/ |
| MG29-1 | 5062 | MG29-1- | /AltR1/rUrArArUrUrUr |
| sgRNA | | mTRAC- | CrUrArCrUrGrUrUrGrUr |
| targeting | | sgRNA-G1 | ArGrArUrGrGrCrArCrAr |
| mouse TRAC | | | UrUrGrArUrUrUrGrGrGr |
| | | | ArGrUrCrArA/AltR2/ |
| MG29-1 | 5063 | MG29-1- | /AltR1/rUrArArUrUrUr |
| sgRNA | | mTRAC- | CrUrArCrUrGrUrUrGrUr |
| targeting | | sgRNA-H1 | ArGrArUrGrGrArGrUrCr |
| mouse TRAC | | | ArArArGrUrCrGrGrUrGr |
| | | | ArArCrArGrG/AltR2/ |
| MG29-1 | 5064 | MG29-1- | /AltR1/rUrArArUrUrUr |
| sgRNA | | mTRAC- | CrUrArCrUrGrUrUrGrUr |
| targeting | | sgRNA-A2 | ArGrArUrArArCrUrGrGr |
| mouse TRAC | | | UrArCrArCrArGrCrArGr |
| | | | GrUrUrCrUrG/AltR2/ |
| MG29-1 | 5065 | MG29-1- | /AltR1/rUrArArUrUrUr |
| sgRNA | | mTRAC- | CrUrArCrUrGrUrUrGrUr |
| targeting | | sgRNA-B2 | ArGrArUrArCrUrGrGrUr |
| mouse TRAC | | | ArCrArCrArGrCrArGrGr |
| | | | UrUrCrUrGrG/AltR2/ |
| MG29-1 | 5066 | MG29-1- | /AltR1/rUrArArUrUrUr |
| sgRNA | | mTRAC- | CrUrArCrUrGrUrUrGrUr |
| targeting | | sgRNA-C2 | ArGrArUrUrUrUrUrCrCr |
| mouse TRAC | | | CrUrUrUrUrArGrArCrGr |
| | | | UrUrCrCrCrU/AltR2/ |
| MG29-1 | 5067 | MG29-1- | /AltR1/rUrArArUrUrUr |
| sgRNA | | mTRAC- | CrUrArCrUrGrUrUrGrUr |
| targeting | | sgRNA-D2 | ArGrArUrCrCrCrUrUrUr |
| mouse TRAC | | | UrArGrArCrGrUrUrCrCr |
| | | | CrUrGrUrGrA/AltR2/ |
| MG29-1 | 5068 | MG29-1- | /AltR1/rUrArArUrUrUr |
| sgRNA | | mTRAC- | CrUrArCrUrGrUrUrGrUr |
| targeting | | sgRNA-E2 | ArGrArUrCrCrUrUrUrUr |
| mouse TRAC | | | ArGrArCrGrUrUrCrCrCr |
| | | | UrGrUrGrArU/AltR2/ |
| MG29-1 | 5069 | MG29-1- | /AltR1/rUrArArUrUrUr |
| sgRNA | | mTRAC- | CrUrArCrUrGrUrUrGrUr |
| targeting | | sgRNA-F2 | ArGrArUrArGrArCrGrUr |
| mouse TRAC | | | UrCrCrCrUrGrUrGrArUr |
| | | | GrCrCrArCrG/AltR2/ |
| MG29-1 | 5070 | MG29-1- | /AltR1/rUrArArUrUrUr |
| sgRNA | | mTRAC- | CrUrArCrUrGrUrUrGrUr |
| targeting | | sgRNA-G2 | ArGrArUrGrArCrGrUrUr |
| mouse TRAC | | | CrCrCrUrGrUrGrArUrGr |
| | | | CrCrArCrGrU/AltR2/ |
| MG29-1 | 5071 | MG29-1- | /AltR1/rUrArArUrUrUr |
| sgRNA | | mTRAC- | CrUrArCrUrGrUrUrGrUr |
| targeting | | sgRNA-H2 | ArGrArUrArArArGrCrUr |
| mouse TRAC | | | UrUrUrCrUrCrArGrUrCr |
| | | | ArArCrGrUrG/AltR2/ |
| MG29-1 | 5072 | MG29-1- | /AltR1/rUrArArUrUrUr |
| sgRNA | | mTRAC- | CrUrArCrUrGrUrUrGrUr |
| targeting | | sgRNA-A3 | ArGrArUrCrUrCrArGrUr |
| mouse TRAC | | | CrArArCrGrUrGrGrCrAr |
| | | | UrCrArCrArG/AltR2/ |
| MG29-1 | 5073 | MG29-1- | /AltR1/rUrArArUrUrUr |
| sgRNA | | mTRAC- | CrUrArCrUrGrUrUrGrUr |
| targeting | | sgRNA-B3 | ArGrArUrUrCrArGrUrCr |
| mouse TRAC | | | ArArCrGrUrGrGrCrArUr |
| | | | CrArCrArGrG/AltR2/ |
| MG29-1 | 5074 | MG29-1- | /AltR1/rUrArArUrUrUr |
| sgRNA | | mTRAC- | CrUrArCrUrGrUrUrGrUr |
| targeting | | sgRNA-C3 | ArGrArUrArCrArGrArUr |
| mouse TRAC | | | ArUrGrArArCrCrUrArAr |
| | | | ArCrUrUrUrC/AltR2/ |
| MG29-1 | 5075 | MG29-1- | /AltR1/rUrArArUrUrUr |
| sgRNA | | mTRAC- | CrUrArCrUrGrUrUrGrUr |
| targeting | | sgRNA-D3 | ArGrArUrCrArGrArUrAr |
| mouse TRAC | | | UrGrArArCrCrUrArArAr |
| | | | CrUrUrUrCrA/AltR2/ |
| MG29-1 | 5076 | MG29-1- | /AltR1/rUrArArUrUrUr |
| sgRNA | | mTRAC- | CrUrArCrUrGrUrUrGrUr |
| targeting | | sgRNA-E3 | ArGrArUrArArArArCrCr |
| mouse TRAC | | | UrGrUrCrArGrUrUrArUr |
| | | | GrGrGrArCrU/AltR2/ |
| MG29-1 | 5077 | MG29-1- | /AltR1/rUrArArUrUrUr |
| sgRNA | | mTRAC- | CrUrArCrUrGrUrUrGrUr |
| targeting | | sgRNA-F3 | ArGrArUrArCrCrUrGrCr |
| mouse TRAC | | | UrCrArUrGrArCrGrCrUr |
| | | | GrArGrGrCrU/AltR2/ |
| MG29-1 | 5078 | MG29-1- | /AltR1/rUrArArUrUrUr |
| sgRNA | | mTRAC- | CrUrArCrUrGrUrUrGrUr |
| targeting | | sgRNA-G3 | ArGrArUrArUrGrCrCrUr |
| mouse TRAC | | | UrCrUrUrArCrCrUrCrAr |
| | | | ArCrUrGrGrA/AltR2/ |
| MG29-1 | 5079 | MG29-1- | /AltR1/rUrArArUrUrUr |
| sgRNA | | mTRAC- | CrUrArCrUrGrUrUrGrUr |
| targeting | | sgRNA-H3 | ArGrArUrArGrCrArGrGr |
| mouse TRAC | | | ArGrGrArUrUrCrGrGrAr |
| | | | GrUrCrCrCrA/AltR2/ |
| MG29-1 | 5080 | MG29-1- | /AltR1/rUrArArUrUrUr |
| sgRNA | | mTRAC- | CrUrArCrUrGrUrUrGrUr |
| targeting | | sgRNA-A4 | ArGrArUrUrGrArGrGrAr |
| mouse TRAC | | | ArGrGrUrUrGrCrUrGrGr |
| | | | ArGrArGrCrU/AltR2/ |
| MG29-1 | 5081 | MG29-1- | /AltR1/rUrArArUrUrUr |
| sgRNA | | mTRAC- | CrUrArCrUrGrUrUrGrUr |
| targeting | | sgRNA-B4 | ArGrArUrUrUrGrUrUrUr |
| mouse TRAC | | | UrUrUrUrUrUrUrUrUrUr |
| | | | UrGrCrGrGrG/AltR2/ |
| MG29-1 | 5082 | MG29-1- | /AltR1/rUrArArUrUrUr |
| sgRNA | | mTRAC- | CrUrArCrUrGrUrUrGrUr |
| targeting | | sgRNA-C4 | ArGrArUrUrGrUrUrUrUr |
| mouse TRAC | | | UrUrUrUrUrUrUrUrUrUr |
| | | | GrCrGrGrGrU/AltR2/ |
| MG29-1 | 5083 | MG29-1- | /AltR1/rUrArArUrUrUr |
| sgRNA | | mTRAC- | CrUrArCrUrGrUrUrGrUr |
| targeting | | sgRNA-D4 | ArGrArUrGrUrUrUrUrUr |
| mouse TRAC | | | UrUrUrUrUrUrUrUrUrGr |
| | | | CrGrGrGrUrU/AltR2/ |
| MG29-1 | 5084 | MG29-1- | /AltR1/rUrArArUrUrUr |
| sgRNA | | mTRAC- | CrUrArCrUrGrUrUrGrUr |
| targeting | | sgRNA-E4 | ArGrArUrUrUrUrUrUrUr |
| mouse TRAC | | | UrUrUrUrUrUrUrUrGrCr |
| | | | GrGrGrUrUrU/AltR2/ |
| MG29-1 | 5085 | MG29-1- | /AltR1/rUrArArUrUrUr |
| sgRNA | | mTRAC- | CrUrArCrUrGrUrUrGrUr |
| targeting | | sgRNA-F4 | ArGrArUrUrUrUrUrUrUr |
| mouse TRAC | | | UrUrUrUrGrCrGrGrGrUr |
| | | | UrUrArUrUrU/AltR2/ |
| MG29-1 | 5086 | MG29-1- | /AltR1/rUrArArUrUrUr |
| sgRNA | | mTRAC- | CrUrArCrUrGrUrUrGrUr |
| targeting | | sgRNA-G4 | ArGrArUrUrUrUrUrUrUr |
| mouse TRAC | | | UrUrUrGrCrGrGrGrUrUr |
| | | | UrArUrUrUrU/AltR2/ |
| MG29-1 | 5087 | MG29-1- | /AltR1/rUrArArUrUrUr |
| sgRNA | | mTRAC- | CrUrArCrUrGrUrUrGrUr |
| targeting | | sgRNA-H4 | ArGrArUrUrUrUrUrUrUr |
| mouse TRAC | | | UrUrGrCrGrGrGrUrUrUr |
| | | | ArUrUrUrUrU/AltR2/ |
| MG29-1 | 5088 | MG29-1- | /AltR1/rUrArArUrUrUr |
| sgRNA | | mTRAC- | CrUrArCrUrGrUrUrGrUr |
| targeting | | sgRNA-A5 | ArGrArUrUrUrUrUrUrUr |
| mouse TRAC | | | UrGrCrGrGrGrUrUrUrAr |
| | | | UrUrUrUrUrU/AltR2/ |
| MG29-1 | 5089 | MG29-1- | /AltR1/rUrArArUrUrUr |
| sgRNA | | mTRAC- | CrUrArCrUrGrUrUrGrUr |
| targeting | | sgRNA-B5 | ArGrArUrUrUrUrUrUrUr |
| mouse TRAC | | | GrCrGrGrGrUrUrUrArUr |
| | | | UrUrUrUrUrU/AltR2/ |
| MG29-1 | 5090 | MG29-1- | /AltR1/rUrArArUrUrUr |
| sgRNA | | mTRAC- | CrUrArCrUrGrUrUrGrUr |
| targeting | | sgRNA-C5 | ArGrArUrUrUrUrUrUrGr |
| mouse TRAC | | | CrGrGrGrUrUrUrArUrUr |
| | | | UrUrUrUrUrA/AltR2/ |
| MG29-1 | 5091 | MG29-1- | /AltR1/rUrArArUrUrUr |
| sgRNA | | mTRAC- | CrUrArCrUrGrUrUrGrUr |
| targeting | | sgRNA-D5 | ArGrArUrUrUrUrUrGrCr |
| mouse TRAC | | | GrGrGrUrUrUrArUrUrUr |
| | | | UrUrUrUrArA/AltR2/ |
| MG29-1 | 5092 | MG29-1- | /AltR1/rUrArArUrUrUr |
| sgRNA | | mTRAC- | CrUrArCrUrGrUrUrGrUr |
| targeting | | sgRNA-E5 | ArGrArUrUrUrUrGrCrGr |
| mouse TRAC | | | GrGrUrUrUrArUrUrUrUr |
| | | | UrUrUrArArG/AltR2/ |
| MG29-1 | 5093 | MG29-1- | /AltR1/rUrArArUrUrUr |
| sgRNA | | mTRAC- | CrUrArCrUrGrUrUrGrUr |
| targeting | | sgRNA-F5 | ArGrArUrUrUrGrCrGrGr |
| mouse TRAC | | | GrUrUrUrArUrUrUrUrUr |
| | | | UrUrArArGrC/AltR2/ |
| MG29-1 | 5094 | MG29-1- | /AltR1/rUrArArUrUrUr |
| sgRNA | | mTRAC- | CrUrArCrUrGrUrUrGrUr |
| targeting | | sgRNA-G5 | ArGrArUrUrGrCrGrGrGr |
| mouse TRAC | | | UrUrUrArUrUrUrUrUrUr |
| | | | UrArArGrCrA/AltR2/ |
| MG29-1 | 5095 | MG29-1- | /AltR1/rUrArArUrUrUr |
| sgRNA | | mTRAC- | CrUrArCrUrGrUrUrGrUr |
| targeting | | sgRNA-H5 | ArGrArUrGrCrGrGrGrUr |
| mouse TRAC | | | UrUrArUrUrUrUrUrUrUr |
| | | | ArArGrCrArU/AltR2 |
| MG29-1 | 5096 | MG29-1- | /AltR1/rUrArArUrUrUr |
| sgRNA | | mTRAC- | CrUrArCrUrGrUrUrGrUr |
| targeting | | sgRNA-A6 | ArGrArUrCrGrGrGrUrUr |
| mouse TRAC | | | UrArUrUrUrUrUrUrUrAr |
| | | | ArGrCrArUrC/AltR2/ |
| MG29-1 | 5097 | MG29-1- | /AltR1/rUrArArUrUrUr |
| sgRNA | | mTRAC- | CrUrArCrUrGrUrUrGrUr |
| targeting | | sgRNA-B6 | ArGrArUrUrUrUrUrUrUr |
| mouse TRAC | | | UrArArGrCrArUrCrCrAr |
| | | | UrGrArArGrA/AltR2/ |
| MG29-1 | 5098 | MG29-1- | /AltR1/rUrArArUrUrUr |
| sgRNA | | mTRAC- | CrUrArCrUrGrUrUrGrUr |
| targeting | | sgRNA-C6 | ArGrArUrUrUrUrArArGr |
| mouse TRAC | | | CrArUrCrCrArUrGrArAr |
| | | | GrArArArUrG/AltR2/ |
| MG29-1 | 5099 | MG29-1- | /AltR1/rUrArArUrUrUr |
| sgRNA | | mTRAC- | CrUrArCrUrGrUrUrGrUr |
| targeting | | sgRNA-D6 | ArGrArUrUrUrArArGrCr |
| mouse TRAC | | | ArUrCrCrArUrGrArArGr |
| | | | ArArArUrGrC/AltR2/ |
| MG29-1 | 5100 | MG29-1- | /AltR1/rUrArArUrUrUr |
| sgRNA | | mTRAC- | CrUrArCrUrGrUrUrGrUr |
| targeting | | sgRNA-E6 | ArGrArUrUrArArGrCrAr |
| mouse TRAC | | | UrCrCrArUrGrArArGrAr |
| | | | ArArUrGrCrA/AltR2/ |
| MG29-1 | 5101 | MG29-1- | /AltR1/rUrArArUrUrUr |
| sgRNA | | mTRAC- | CrUrArCrUrGrUrUrGrUr |
| targeting | | sgRNA-F6 | ArGrArUrArArGrCrArUr |
| mouse TRAC | | | CrCrArUrGrArArGrArAr |
| | | | ArUrGrCrArU/AltR2/ |
| MG29-1 | 5102 | MG29-1- | /AltR1/rUrArArUrUrUr |
| sgRNA | | mTRAC- | CrUrArCrUrGrUrUrGrUr |
| targeting | | sgRNA-G6 | ArGrArUrArGrCrArUrCr |
| mouse TRAC | | | CrArUrGrArArGrArArAr |
| | | | UrGrCrArUrA/AltR2/ |
| MG29-1 | 5103 | MG29-1- | /AltR1/rUrArArUrUrUr |
| sgRNA | | mTRAC- | CrUrArCrUrGrUrUrGrUr |
| targeting | | sgRNA-H6 | ArGrArUrArUrCrArArGr |
| mouse TRAC | | | GrUrGrUrArGrArArArUr |
| | | | UrArUrCrUrC/AltR2/ |
| MG29-1 | 5104 | MG29-1- | /AltR1/rUrArArUrUrUr |
| sgRNA | | mTRAC- | CrUrArCrUrGrUrUrGrUr |
| targeting | | sgRNA-A7 | ArGrArUrGrGrUrUrUrUr |
| mouse TRAC | | | UrCrUrGrArArUrCrArCr |
| | | | CrUrUrUrArA/AltR2/ |
| MG29-1 | 5105 | MG29-1- | /AltR1/rUrArArUrUrUr |
| sgRNA | | mTRAC- | CrUrArCrUrGrUrUrGrUr |
| targeting | | sgRNA-B7 | ArGrArUrGrUrUrUrUrUr |
| mouse TRAC | | | CrUrGrArArUrCrArCrCr |
| | | | UrUrUrArArU/AltR2/ |
| MG29-1 | 5106 | MG29-1- | /AltR1/rUrArArUrUrUr |
| sgRNA | | mTRAC- | CrUrArCrUrGrUrUrGrUr |
| targeting | | sgRNA-C7 | ArGrArUrUrCrUrGrArAr |
| mouse TRAC | | | UrCrArCrCrUrUrUrArAr |
| | | | UrGrArUrGrU/AltR2/ |
| MG29-1 | 5107 | MG29-1- | /AltR1/rUrArArUrUrUr |
| sgRNA | | mTRAC- | CrUrArCrUrGrUrUrGrUr |
| targeting | | sgRNA-D7 | ArGrArUrCrUrGrArArUr |
| mouse TRAC | | | CrArCrCrUrUrUrArArUr |
| | | | GrArUrGrUrC/AltR2/ |
| MG29-1 | 5108 | MG29-1- | /AltR1/rUrArArUrUrUr |
| sgRNA | | mTRAC- | CrUrArCrUrGrUrUrGrUr |
| targeting | | sgRNA-E7 | ArGrArUrUrGrArArUrCr |
| mouse TRAC | | | ArCrCrUrUrUrArArUrGr |
| | | | ArUrGrUrCrA/AltR2/ |
| MG29-1 | 5109 | MG29-1- | /AltR1/rUrArArUrUrUr |
| sgRNA | | mTRAC- | CrUrArCrUrGrUrUrGrUr |
| targeting | | sgRNA-F7 | ArGrArUrArUrGrArUrGr |
| mouse TRAC | | | UrCrArUrGrGrArCrArGr |
| | | | CrArGrArArU/AltR2/ |
| MG29-1 | 5110 | MG29-1- | /AltR1/rUrArArUrUrUr |
| sgRNA | | mTRAC- | CrUrArCrUrGrUrUrGrUr |
| targeting | | sgRNA-G7 | ArGrArUrUrArCrArCrCr |
| mouse TRAC | | | UrUrGrArUrGrArArArGr |
| | | | ArGrUrArArU/AltR2/ |
| MG29-1 | 5111 | MG29-1- | /AltR1/rUrArArUrUrUr |
| sgRNA | | mTRAC- | CrUrArCrUrGrUrUrGrUr |
| targeting | | sgRNA-H7 | ArGrArUrUrUrCrArUrGr |
| mouse TRAC | | | GrArUrGrCrUrUrArArAr |
| | | | ArArArArUrA/AltR2/ |
| MG29-1 | 5112 | MG29-1- | /AltR1/rUrArArUrUrUr |
| sgRNA | | mTRAC- | CrUrArCrUrGrUrUrGrUr |
| targeting | | sgRNA-A8 | ArGrArUrUrUrUrUrArCr |
| mouse TRAC | | | ArArCrArUrUrCrUrCrCr |
| | | | ArArGrArGrA/AltR2/ |
| MG29-1 | 5113 | MG29-1- | /AltR1/rUrArArUrUrUr |
| sgRNA | | mTRAC- | CrUrArCrUrGrUrUrGrUr |
| targeting | | sgRNA-B8 | ArGrArUrUrUrUrArCrAr |
| mouse TRAC | | | ArCrArUrUrCrUrCrCrAr |
| | | | ArGrArGrArU/AltR2/ |
| MG29-1 | 5114 | MG29-1- | /AltR1/rUrArArUrUrUr |
| sgRNA | | mTRAC- | CrUrArCrUrGrUrUrGrUr |
| targeting | | sgRNA-C8 | ArGrArUrUrUrArCrArAr |
| mouse TRAC | | | CrArUrUrCrUrCrCrArAr |
| | | | GrArGrArUrU/AltR2/ |
| MG29-1 | 5115 | MG29-1- | /AltR1/rUrArArUrUrUr |
| sgRNA | | mTRAC- | CrUrArCrUrGrUrUrGrUr |
| targeting | | sgRNA-D8 | ArGrArUrUrArCrArArCr |
| mouse TRAC | | | ArUrUrCrUrCrCrArArGr |
| | | | ArGrArUrUrU/AltR2/ |
| MG29-1 | 5116 | MG29-1- | /AltR1/rUrArArUrUrUr |
| sgRNA | | mTRAC- | CrUrArCrUrGrUrUrGrUr |
| targeting | | sgRNA-E8 | ArGrArUrArCrArArCrAr |
| mouse TRAC | | | UrUrCrUrCrCrArArGrAr |
| | | | GrArUrUrUrU/AltR2/ |
| MG29-1 | 5117 | MG29-1- | /AltR1/rUrArArUrUrUr |
| sgRNA | | mTRAC- | CrUrArCrUrGrUrUrGrUr |
| targeting | | sgRNA-F8 | ArGrArUrCrArArCrArUr |
| mouse TRAC | | | UrCrUrCrCrArArGrArGr |
| | | | ArUrUrUrUrA/AltR2/ |
| MG29-1 | 5118 | MG29-1- | /AltR1/rUrArArUrUrUr |
| sgRNA | | mTRAC- | CrUrArCrUrGrUrUrGrUr |
| targeting | | sgRNA-G8 | ArGrArUrArCrArGrGrGr |
| mouse TRAC | | | GrArGrUrCrUrGrCrCrAr |
| | | | UrGrGrGrGrG/AltR2/ |
| MG29-1 | 5119 | MG29-1- | /AltR1/rUrArArUrUrUr |
| sgRNA | | mTRAC- | CrUrArCrUrGrUrUrGrUr |
| targeting | | sgRNA-H8 | ArGrArUrCrArGrGrGrGr |
| mouse TRAC | | | ArGrUrCrUrGrCrCrArUr |
| | | | GrGrGrGrGrA/AltR2/ |
| MG29-1 | 5120 | MG29-1- | /AltR1/rUrArArUrUrUr |
| sgRNA | | mTRAC- | CrUrArCrUrGrUrUrGrUr |
| targeting | | sgRNA-A9 | ArGrArUrUrUrGrUrGrAr |
| mouse TRAC | | | ArUrGrGrUrCrArGrCrAr |
| | | | GrCrArGrUrG/AltR2/ |
| MG29-1 | 5121 | MG29-1- | /AltR1/rUrArArUrUrUr |
| sgRNA | | mTRAC- | CrUrArCrUrGrUrUrGrUr |
| targeting | | sgRNA-B9 | ArGrArUrUrGrUrGrArAr |
| mouse TRAC | | | UrGrGrUrCrArGrCrArGr |
| | | | CrArGrUrGrA/AltR2/ |
| MG29-1 | 5122 | MG29-1- | /AltR1/rUrArArUrUrUr |
| sgRNA | | mTRAC- | CrUrArCrUrGrUrUrGrUr |
| targeting | | sgRNA-C9 | ArGrArUrGrUrGrArArUr |
| mouse TRAC | | | GrGrUrCrArGrCrArGrCr |
| | | | ArGrUrGrArG/AltR2/ |
| MG29-1 | 5123 | MG29-1- | /AltR1/rUrArArUrUrUr |
| sgRNA | | mTRAC- | CrUrArCrUrGrUrUrGrUr |
| targeting | | sgRNA-D9 | ArGrArUrUrGrArArUrGr |
| mouse TRAC | | | GrUrCrArGrCrArGrCrAr |
| | | | GrUrGrArGrG/AltR2/ |
| MG29-1 | 5124 | MG29-1- | /AltR1/rUrArArUrUrUr |
| sgRNA | | mTRAC- | CrUrArCrUrGrUrUrGrUr |
| targeting | | sgRNA-E9 | ArGrArUrGrGrCrUrUrGr |
| mouse TRAC | | | ArArGrArArGrGrArGrCr |
| | | | GrGrArGrGrG/AltR2/ |
| MG29-1 | 5125 | MG29-1- | /AltR1/rUrArArUrUrUr |
| sgRNA | | mTRAC- | CrUrArCrUrGrUrUrGrUr |
| targeting | | sgRNA-F9 | ArGrArUrGrCrUrUrGrAr |
| mouse TRAC | | | ArGrArArGrGrArGrCrGr |
| | | | GrArGrGrGrG/AltR2/ |
| DNA sequence | 5126 | MG29-1- | ACTCCCAAATCAATGTGCCG |
| of TRAC | | mTRAC- | AA |
| target site | | target | |
| | | site-A1 | |
| DNA sequence | 5127 | MG29-1- | AAGATATCTTGGCAGGTGAA |
| of TRAC | | mTRAC- | GC |
| target site | | target | |
| | | site-B1 | |
| DNA sequence | 5128 | MG29-1- | ATGTCCAGCACAGTTTTGTC |
| of TRAC | | mTRAC- | AG |
| target site | | target | |
| | | site-C1 | |
| DNA sequence | 5129 | MG29-1- | GTCAGTGATGAACGTTCCAG |
| of TRAC | | mTRAC- | AT |
| target site | | target | |
| | | site-D1 | |
| DNA sequence | 5130 | MG29-1- | TCAGTGATGAACGTTCCAGA |
| of TRAC | | mTRAC- | TT |
| target site | | target | |
| | | site-E1 | |
| DNA sequence | 5131 | MG29-1- | CGGCACATTGATTTGGGAGT |
| of TRAC | | mTRAC- | CA |
| target site | | target | |
| | | site-F1 | |
| DNA sequence | 5132 | MG29-1- | GGCACATTGATTTGGGAGTC |
| of TRAC | | mTRAC- | AA |
| target site | | target | |
| | | site-G1 | |
| DNA sequence | 5133 | MG29-1- | GGAGTCAAAGTCGGTGAACA |
| of TRAC | | mTRAC- | GG |
| target site | | target | |
| | | site-H1 | |
| DNA sequence | 5134 | MG29-1- | AACTGGTACACAGCAGGTTC |
| of TRAC | | mTRAC- | TG |
| target site | | target | |
| | | site-A2 | |
| DNA sequence | 5135 | MG29-1- | ACTGGTACACAGCAGGTTCT |
| of TRAC | | mTRAC- | GG |
| target site | | target | |
| | | site-B2 | |
| DNA sequence | 5136 | MG29-1- | TTTTCCCTTTTAGACGTTCC |
| of TRAC | | mTRAC- | CT |
| target site | | target | |
| | | site-C2 | |
| DNA sequence | 5137 | MG29-1- | CCCTTTTAGACGTTCCCTGT |
| of TRAC | | mTRAC- | GA |
| target site | | target | |
| | | site-D2 | |
| DNA sequence | 5138 | MG29-1- | CCTTTTAGACGTTCCCTGTG |
| of TRAC | | mTRAC- | AT |
| target site | | target | |
| | | site-E2 | |
| DNA sequence | 5139 | MG29-1- | AGACGTTCCCTGTGATGCCA |
| of TRAC | | mTRAC- | CG |
| target site | | target | |
| | | site-F2 | |
| DNA sequence | 5140 | MG29-1- | GACGTTCCCTGTGATGCCAC |
| of TRAC | | mTRAC- | GT |
| target site | | target | |
| | | site-G2 | |
| DNA sequence | 5141 | MG29-1- | AAAGCTTTTCTCAGTCAACG |
| of TRAC | | mTRAC- | TG |
| target site | | target | |
| | | site-H2 | |
| DNA sequence | 5142 | MG29-1- | CTCAGTCAACGTGGCATCAC |
| of TRAC | | mTRAC- | AG |
| target site | | target | |
| | | site-A3 | |
| DNA sequence | 5143 | MG29-1- | TCAGTCAACGTGGCATCACA |
| of TRAC | | mTRAC- | GG |
| target site | | target | |
| | | site-B3 | |
| DNA sequence | 5144 | MG29-1- | ACAGATATGAACCTAAACTT |
| of TRAC | | mTRAC- | TC |
| target site | | target | |
| | | site-C3 | |
| DNA sequence | 5145 | MG29-1- | CAGATATGAACCTAAACTTT |
| of TRAC | | mTRAC- | CA |
| target site | | target | |
| | | site-D3 | |
| DNA sequence | 5146 | MG29-1- | AAAACCTGTCAGTTATGGGA |
| of TRAC | | mTRAC- | CT |
| target site | | target | |
| | | site-E3 | |
| DNA sequence | 5147 | MG29-1- | ACCTGCTCATGACGCTGAGG |
| of TRAC | | mTRAC- | CT |
| target site | | target | |
| | | site-F3 | |
| DNA sequence | 5148 | MG29-1- | ATGCCTTCTTACCTCAACTG |
| of TRAC | | mTRAC- | GA |
| target site | | target | |
| | | site-G3 | |
| DNA sequence | 5149 | MG29-1- | AGCAGGAGGATTCGGAGTCC |
| of TRAC | | mTRAC- | CA |
| target site | | target | |
| | | site-H3 | |
| DNA sequence | 5150 | MG29-1- | TGAGGAAGGTTGCTGGAGAG |
| of TRAC | | mTRAC- | CT |
| target site | | target | |
| | | site-A4 | |
| DNA sequence | 5151 | MG29-1- | TTGTTTTTTTTTTTTTTGCG |
| of TRAC | | mTRAC- | GG |
| target site | | target | |
| | | site-B4 | |
| DNA sequence | 5152 | MG29-1- | TGTTTTTTTTTTTTTTGCGG |
| of TRAC | | mTRAC- | GT |
| target site | | target | |
| | | site-C4 | |
| DNA sequence | 5153 | MG29-1- | GTTTTTTTTTTTTTTGCGGG |
| of TRAC | | mTRAC- | TT |
| target site | | target | |
| | | site-D4 | |
| DNA sequence | 5154 | MG29-1- | TTTTTTTTTTTTTTGCGGGT |
| of TRAC | | mTRAC- | TT |
| target site | | target | |
| | | site-E4 | |
| DNA sequence | 5155 | MG29-1- | TTTTTTTTTTGCGGGTTTAT |
| of TRAC | | mTRAC- | TT |
| target site | | target | |
| | | site-F4 | |
| DNA sequence | 5156 | MG29-1- | TTTTTTTTTGCGGGTTTATT |
| of TRAC | | mTRAC- | TT |
| target site | | target | |
| | | site-G4 | |
| DNA sequence | 5157 | MG29-1- | TTTTTTTTGCGGGTTTATTT |
| of TRAC | | mTRAC- | TT |
| target site | | target | |
| | | site-H4 | |
| Guide Target | SEQ | Guide Name | SEQUENCE |
| | ID | | |
| | NO | | |
| DNA sequence | 5158 | MG29-1- | TTTTTTTGCGGGTTTATTTT |
| of TRAC | | mTRAC- | TT |
| target site | | target | |
| | | site-A5 | |
| DNA sequence | 5159 | MG29-1- | TTTTTTGCGGGTTTATTTTT |
| of TRAC | | mTRAC- | TT |
| target site | | target | |
| | | site-B5 | |
| DNA sequence | 5160 | MG29-1- | TTTTTGCGGGTTTATTTTTT |
| of TRAC | | mTRAC- | TA |
| target site | | target | |
| | | site-C5 | |
| DNA sequence | 5161 | MG29-1- | TTTTGCGGGTTTATTTTTTT |
| of TRAC | | mTRAC- | AA |
| target site | | target | |
| | | site-D5 | |
| DNA sequence | 5162 | MG29-1- | TTTGCGGGTTTATTTTTTTA |
| of TRAC | | mTRAC- | AG |
| target site | | target | |
| | | site-E5 | |
| DNA sequence | 5163 | MG29-1- | TTGCGGGTTTATTTTTTTAA |
| of TRAC | | mTRAC- | GC |
| target site | | target | |
| | | site-F5 | |
| DNA sequence | 5164 | MG29-1- | TGCGGGTTTATTTTTTTAAG |
| of TRAC | | mTRAC- | CA |
| target site | | target | |
| | | site-G5 | |
| DNA sequence | 5165 | MG29-1- | GCGGGTTTATTTTTTTAAGC |
| of TRAC | | mTRAC- | AT |
| target site | | target | |
| | | site-H5 | |
| DNA sequence | 5166 | MG29-1- | CGGGTTTATTTTTTTAAGCA |
| of TRAC | | mTRAC- | TC |
| target site | | target | |
| | | site-A6 | |
| DNA sequence | 5167 | MG29-1- | TTTTTTTAAGCATCCATGAA |
| of TRAC | | mTRAC- | GA |
| target site | | target | |
| | | site-B6 | |
| DNA sequence | 5168 | MG29-1- | TTTAAGCATCCATGAAGAAA |
| of TRAC | | mTRAC- | TG |
| target site | | target | |
| | | site-C6 | |
| DNA sequence | 5169 | MG29-1- | TTAAGCATCCATGAAGAAAT |
| of TRAC | | mTRAC- | GC |
| target site | | target | |
| | | site-D6 | |
| DNA sequence | 5170 | MG29-1- | TAAGCATCCATGAAGAAATG |
| of TRAC | | mTRAC- | CA |
| target site | | target | |
| | | site-E6 | |
| DNA sequence | 5171 | MG29-1- | AAGCATCCATGAAGAAATGC |
| of TRAC | | mTRAC- | AT |
| target site | | target | |
| | | site-F6 | |
| DNA sequence | 5172 | MG29-1- | AGCATCCATGAAGAAATGCA |
| of TRAC | | mTRAC- | TA |
| target site | | target | |
| | | site-G6 | |
| DNA sequence | 5173 | MG29-1- | ATCAAGGTGTAGAAATTATC |
| of TRAC | | mTRAC- | TC |
| target site | | target | |
| | | site-H6 | |
| DNA sequence | 5174 | MG29-1- | GGTTTTTCTGAATCACCTTT |
| of TRAC | | mTRAC- | AA |
| target site | | target | |
| | | site-A7 | |
| DNA sequence | 5175 | MG29-1- | GTTTTTCTGAATCACCTTTA |
| of TRAC | | mTRAC- | AT |
| target site | | target | |
| | | site-B7 | |
| DNA sequence | 5176 | MG29-1- | TCTGAATCACCTTTAATGAT |
| of TRAC | | mTRAC- | GT |
| target site | | target | |
| | | site-C7 | |
| DNA sequence | 5177 | MG29-1- | CTGAATCACCTTTAATGATG |
| of TRAC | | mTRAC- | TC |
| target site | | target | |
| | | site-D7 | |
| DNA sequence | 5178 | MG29-1- | TGAATCACCTTTAATGATGT |
| of TRAC | | mTRAC- | CA |
| target site | | target | |
| | | site-E7 | |
| DNA sequence | 5179 | MG29-1- | ATGATGTCATGGACAGCAGA |
| of TRAC | | mTRAC- | AT |
| target site | | target | |
| | | site-F7 | |
| DNA sequence | 5180 | MG29-1- | TACACCTTGATGAAAGAGTA |
| of TRAC | | mTRAC- | AT |
| target site | | target | |
| | | site-G7 | |
| DNA sequence | 5181 | MG29-1- | TTCATGGATGCTTAAAAAAA |
| of TRAC | | mTRAC- | TA |
| target site | | target | |
| | | site-H7 | |
| DNA sequence | 5182 | MG29-1- | TTTTACAACATTCTCCAAGA |
| of TRAC | | mTRAC- | GA |
| target site | | target | |
| | | site-A8 | |
| DNA sequence | 5183 | MG29-1- | TTTACAACATTCTCCAAGAG |
| of TRAC | | mTRAC- | AT |
| target site | | target | |
| | | site-B8 | |
| DNA sequence | 5184 | MG29-1- | TTACAACATTCTCCAAGAGA |
| of TRAC | | mTRAC- | TT |
| target site | | target | |
| | | site-C8 | |
| DNA sequence | 5185 | MG29-1- | TACAACATTCTCCAAGAGAT |
| of TRAC | | mTRAC- | TT |
| target site | | target | |
| | | site-D8 | |
| DNA sequence | 5186 | MG29-1- | ACAACATTCTCCAAGAGATT |
| of TRAC | | mTRAC- | TT |
| target site | | target | |
| | | site-E8 | |
| DNA sequence | 5187 | MG29-1- | CAACATTCTCCAAGAGATTT |
| of TRAC | | mTRAC- | TA |
| target site | | target | |
| | | site-F8 | |
| DNA sequence | 5188 | MG29-1- | ACAGGGGAGTCTGCCATGGG |
| of TRAC | | mTRAC- | GG |
| target site | | target | |
| | | site-G8 | |
| DNA sequence | 5189 | MG29-1- | CAGGGGAGTCTGCCATGGGG |
| of TRAC | | mTRAC- | GA |
| target site | | target | |
| | | site-H8 | |
| DNA sequence | 5190 | MG29-1- | TTGTGAATGGTCAGCAGCAG |
| of TRAC | | mTRAC- | TG |
| target site | | target | |
| | | site-A9 | |
| DNA sequence | 5191 | MG29-1- | TGTGAATGGTCAGCAGCAGT |
| of TRAC | | mTRAC- | GA |
| target site | | target | |
| | | site-B9 | |
| DNA sequence | 5192 | MG29-1- | GTGAATGGTCAGCAGCAGTG |
| of TRAC | | mTRAC- | AG |
| target site | | target | |
| | | site-C9 | |
| DNA sequence | 5193 | MG29-1- | TGAATGGTCAGCAGCAGTGA |
| of TRAC | | mTRAC- | GG |
| target site | | target | |
| | | site-D9 | |
| DNA sequence | 5194 | MG29-1- | GGCTTGAAGAAGGAGCGGAG |
| of TRAC | | mTRAC- | GG |
| target site | | target | |
| | | site-E9 | |
| DNA sequence | 5195 | MG29-1- | GCTTGAAGAAGGAGCGGAGG |
| of TRAC | | mTRAC- | GG |
| target site | | target | |
| | | site-F9 | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

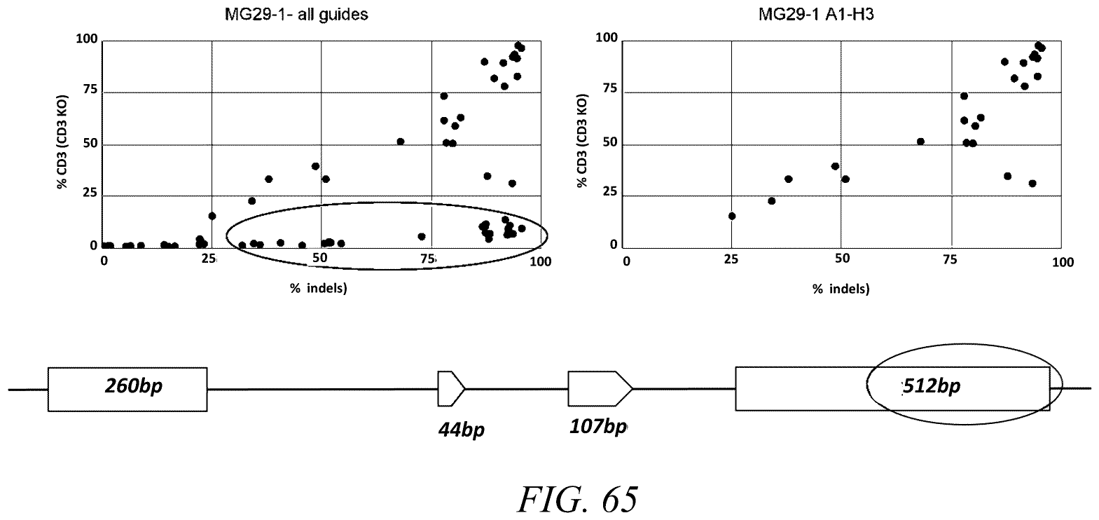

For analysis by flow cytometry, 3 days post-nucleofection, 100,000 mouse T cells were stained with anti-mouse CD3 antibody (Clone 17A2, Invitrogen 11-0032-82) for 30 minutes at 4C and analyzed on an Attune Nxt flow cytometer. Guides at the 3′ end of the mouse TRAC gene have high levels of indels but fail to produce a knock-out of TRAC. Surprisingly and unexpectedly, the phenotype does not correlate with the genotype near the end of the gene, likely due to the retention of function of truncated alleles and the failure of nonsense-mediated decay pathways to remove prematurely truncated out-of-frame mRNAs (FIG. 65).

### Example 19—Gene Editing Outcomes at the DNA Level for Mouse TRBC1 and TRBC2

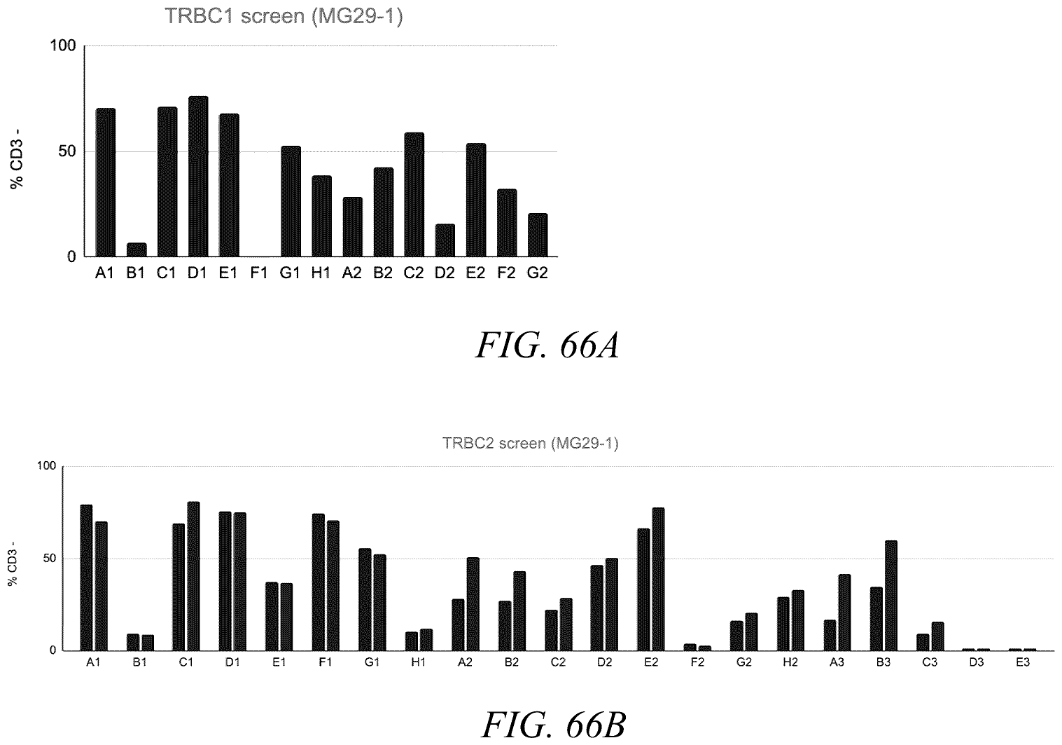

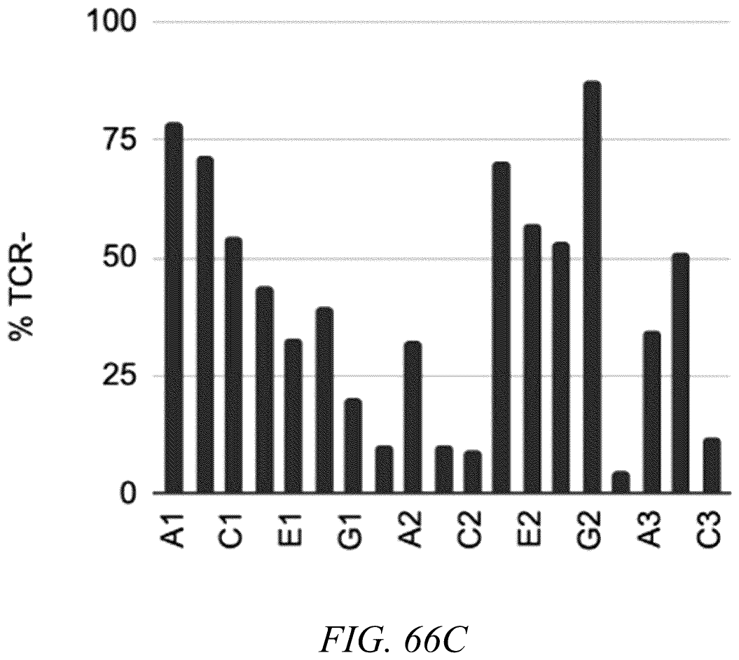

Primary T cells were purified from C57BL/6 mouse spleens. Nucleofection of MG29-1 RNPs (104 pmol protein/120 pmol guide) (TRBC1: SEQ ID NOs: 5196-5210; TRBC2: SEQ ID NOs: 5226-5246) was performed into T cells (200,000) using the Lonza 4D electroporator and 100 pmol transfection enhancer (IDT). Cells were harvested and genomic DNA prepared five days post-transfection. PCR primers appropriate for use in NGS-based DNA sequencing were generated, optimized, and used to amplify the individual target sequences for each guide RNA (TRBC1: SEQ ID NOs: 5211-5225; TRBC2: SEQ ID NOs: 5247-5267). The amplicons were sequenced on an Illumina MiSeq machine and analyzed with a proprietary Python script to measure gene editing (FIGS. 66A and 66B). For analysis by flow cytometry, 3 days post-nucleofection, 100,000 mouse T cells were stained with anti-mouse CD3 antibody (Clone 17A2, Invitrogen 11-0032-82) for 30 minutes at 4C and analyzed on an Attune Nxt flow cytometer.

TABLE 14H
Sequences of Guide RNAs and Sequences
Targeted for Example 45

| | SEQ | | |
|---|---|---|---|
| Guide | ID | Guide | |
| Target | NO | Name | SEQUENCE |
| MG29-1 | 5196 | MG29-1- | /AltR1/rUrArArUrUrUr |
| sgRNA | | mTRBC1- | CrUrArCrUrGrUrUrGrUr |
| targeting | | sgRNA- | ArGrArUrCrUrUrUrCrCr |
| mouse TRBC1 | | A1 | ArGrArGrGrArUrCrUrGr |
| | | | ArGrArArArU/AltR2/ |
| MG29-1 | 5197 | MG29-1- | /AltR1/rUrArArUrUrUr |
| sgRNA | | mTRBC1- | CrUrArCrUrGrUrUrGrUr |
| targeting | | sgRNA- | ArGrArUrCrArGrArGrGr |
| mouse TRBC1 | | B1 | ArUrCrUrGrArGrArArAr |
| | | | UrGrUrGrArC/AltR2/ |
| MG29-1 | 5198 | MG29-1- | /AltR1/rUrArArUrUrUr |
| sgRNA | | mTRBC1- | CrUrArCrUrGrUrUrGrUr |
| targeting | | sgRNA- | ArGrArUrArGrCrCrArUr |
| mouse TRBC1 | | C1 | CrArArArArGrCrArGrAr |
| | | | GrArUrUrGrC/AltR2/ |
| MG29-1 | 5199 | MG29-1- | /AltR1/rUrArArUrUrUr |
| sgRNA | | mTRBC1- | CrUrArCrUrGrUrUrGrUr |
| targeting | | sgRNA- | ArGrArUrArGrArGrGrAr |
| mouse TRBC1 | | D1 | GrGrArCrArArGrUrGrGr |
| | | | CrCrArGrArG/AltR2/ |
| MG29-1 | 5200 | MG29-1- | /AltR1/rUrArArUrUrUr |
| sgRNA | | mTRBC1- | CrUrArCrUrGrUrUrGrUr |
| targeting | | sgRNA- | ArGrArUrGrGrUrGrArGr |
| mouse TRBC1 | | E1 | CrCrCrUrCrUrGrGrCrCr |
| | | | ArCrUrUrGrU/AltR2/ |
| MG29-1 | 5201 | MG29-1- | /AltR1/rUrArArUrUrUr |
| sgRNA | | mTRBC1- | CrUrArCrUrGrUrUrGrUr |
| targeting | | sgRNA- | ArGrArUrGrUrUrUrGrUr |
| mouse TRBC1 | | F1 | UrUrGrCrArArUrCrUrCr |
| | | | UrGrCrUrUrU/AltR2/ |
| MG29-1 | 5202 | MG29-1- | /AltR1/rUrArArUrUrUr |
| sgRNA | | mTRBC1- | CrUrArCrUrGrUrUrGrUr |
| targeting | | sgRNA- | ArGrArUrUrUrUrGrUrUr |
| mouse TRBC1 | | G1 | UrGrCrArArUrCrUrCrUr |
| | | | GrCrUrUrUrU/AltR2/ |
| MG29-1 | 5203 | MG29-1- | /AltR1/rUrArArUrUrUr |
| sgRNA | | mTRBC1- | CrUrArCrUrGrUrUrGrUr |
| targeting | | sgRNA- | ArGrArUrUrUrUrGrCrAr |
| mouse TRBC1 | | H1 | ArUrCrUrCrUrGrCrUrUr |
| | | | UrUrGrArUrG/AltR2/ |
| MG29-1 | 5204 | MG29-1- | /AltR1/rUrArArUrUrUr |
| sgRNA | | mTRBC1- | CrUrArCrUrGrUrUrGrUr |
| targeting | | sgRNA- | ArGrArUrCrArArUrCrUr |
| mouse TRBC1 | | A2 | CrUrGrCrUrUrUrUrGrAr |
| | | | UrGrGrCrUrC/AltR2/ |
| MG29-1 | 5205 | MG29-1- | /AltR1/rUrArArUrUrUr |
| sgRNA | | mTRBC1- | CrUrArCrUrGrUrUrGrUr |
| targeting | | sgRNA- | ArGrArUrGrArUrGrGrCr |
| mouse TRBC1 | | B2 | UrCrArArArCrArArGrGr |
| | | | ArGrArCrCrU/AltR2/ |
| MG29-1 | 5206 | MG29-1- | /AltR1/rUrArArUrUrUr |
| sgRNA | | mTRBC1- | CrUrArCrUrGrUrUrGrUr |
| targeting | | sgRNA- | ArGrArUrArUrGrGrCrUr |
| mouse TRBC1 | | C2 | CrArArArCrArArGrGrAr |
| | | | GrArCrCrUrU/AltR2/ |
| MG29-1 | 5207 | MG29-1- | /AltR1/rUrArArUrUrUr |
| sgRNA | | mTRBC1- | CrUrArCrUrGrUrUrGrUr |
| targeting | | sgRNA- | ArGrArUrUrCrUrCrUrUr |
| mouse TRBC1 | | D2 | CrUrUrUrCrArGrArCrUr |
| | | | GrUrGrGrGrA/AltR2/ |
| MG29-1 | 5208 | MG29-1- | /AltR1/rUrArArUrUrUr |
| sgRNA | | mTRBC1- | CrUrArCrUrGrUrUrGrUr |
| targeting | | sgRNA- | ArGrArUrCrUrGrUrCrAr |
| mouse TRBC1 | | E2 | ArCrArGrCrArUrCrCrUr |
| | | | ArUrCrArArC/AltR2/ |
| MG29-1 | 5209 | MG29-1- | /AltR1/rUrArArUrUrUr |
| sgRNA | | mTRBC1- | CrUrArCrUrGrUrUrGrUr |
| targeting | | sgRNA- | ArGrArUrUrGrUrCrArAr |
| mouse TRBC1 | | F2 | CrArGrCrArUrCrCrUrAr |
| | | | UrCrArArCrA/AltR2/ |
| MG29-1 | 5210 | MG29-1- | /AltR1/rUrArArUrUrUr |
| sgRNA | | mTRBC1- | CrUrArCrUrGrUrUrGrUr |
| targeting | | sgRNA- | ArGrArUrCrCrUrArGrCr |
| mouse TRBC1 | | G2 | ArGrGrArUrCrUrCrArUr |
| | | | ArGrArGrGrA/AltR2/ |
| DNA sequence | 5211 | MG29-1- | CTTTCCAGAGGATCTGAGAA |
| of TRBC1 | | mTRBC1- | AT |
| target site | | target | |
| | | site-A1 | |
| DNA sequence | 5212 | MG29-1- | CAGAGGATCTGAGAAATGTG |
| of TRBC1 | | mTRBC1- | AC |
| target site | | target | |
| | | site-B1 | |
| DNA sequence | 5213 | MG29-1- | AGCCATCAAAAGCAGAGATT |
| of TRBC1 | | mTRBC1- | GC |
| target site | | target | |
| | | site-C1 | |
| DNA sequence | 5214 | MG29-1- | AGAGGAGGACAAGTGGCCAG |
| of TRBC1 | | mTRBC1- | AG |
| target site | | target | |
| | | site-D1 | |
| DNA sequence | 5215 | MG29-1- | GGTGAGCCCTCTGGCCACTT |
| of TRBC1 | | m TRBC1- | GT |
| target site | | target | |
| | | site-E1 | |
| DNA sequence | 5216 | MG29-1- | GTTTGTTTGCAATCTCTGCT |
| of TRBC1 | | mTRBC1- | TT |
| target site | | target | |
| | | site-F1 | |
| DNA sequence | 5217 | MG29-1- | TTTGTTTGCAATCTCTGCTT |
| of TRBC1 | | mTRBC1- | TT |
| target site | | target | |
| | | site-G1 | |
| DNA sequence | 5218 | MG29-1- | TTTGCAATCTCTGCTTTTGA |
| of TRBC1 | | mTRBC1- | TG |
| target site | | target | |
| | | site-H1 | |
| DNA sequence | 5219 | MG29-1- | CAATCTCTGCTTTTGATGGC |
| of TRBC1 | | mTRBC1- | TC |
| target site | | target | |
| | | site-A2 | |
| DNA sequence | 5220 | MG29-1- | GATGGCTCAAACAAGGAGAC |
| of TRBC1 | | mTRBC1- | CT |
| target site | | target | |
| | | site-B2 | |
| DNA sequence | 5221 | MG29-1- | ATGGCTCAAACAAGGAGACC |
| of TRBC1 | | mTRBC1- | TT |
| target site | | target | |
| | | site-C2 | |
| DNA sequence | 5222 | MG29-1- | TCTCTTCTTTCAGACTGTGG |
| of TRBC1 | | mTRBC1- | GA |
| target site | | target | |
| | | site-D2 | |
| DNA sequence | 5223 | MG29-1- | CTGTCAACAGCATCCTATCA |
| of TRBC1 | | mTRBC1- | AC |
| target site | | target | |
| | | site-E2 | |
| DNA sequence | 5224 | MG29-1- | TGTCAACAGCATCCTATCAA |
| of TRBC1 | | mTRBC1- | CA |
| target site | | target | |
| | | site-F2 | |
| DNA sequence | 5225 | MG29-1- | CCTAGCAGGATCTCATAGAG |
| of TRBC1 | | mTRBC1- | GA |
| target site | | target | |
| | | site-G2 | |
| MG29-1 | 5226 | MG29-1- | /AltR1/rUrArArUrUrUr |
| sgRNA | | mTRBC2- | CrUrArCrUrGrUrUrGrUr |
| targeting | | sgRNA- | ArGrArUrCrUrUrUrCrCr |
| mouse TRBC2 | | A1 | ArGrArGrGrArUrCrUrGr |
| | | | ArGrArArArU/AltR2/ |
| MG29-1 | 5227 | MG29-1- | /AltR1/rUrArArUrUrUr |
| sgRNA | | mTRBC2- | CrUrArCrUrGrUrUrGrUr |
| targeting | | sgRNA- | ArGrArUrCrArGrArGrGr |
| mouse TRBC2 | | B1 | ArUrCrUrGrArGrArArAr |
| | | | UrGrUrGrArC/AltR2/ |
| MG29-1 | 5228 | MG29-1- | /AltR1/rUrArArUrUrUr |
| sgRNA | | mTRBC2- | CrUrArCrUrGrUrUrGrUr |
| targeting | | sgRNA- | ArGrArUrArGrCrCrArUr |
| mouse TRBC2 | | C1 | CrArArArArGrCrArGrAr |
| | | | GrArUrUrGrC/AltR2/ |
| MG29-1 | 5229 | MG29-1- | /AltR1/rUrArArUrUrUr |
| sgRNA | | mTRBC2- | CrUrArCrUrGrUrUrGrUr |
| targeting | | sgRNA- | ArGrArUrArGrArGrGrAr |
| mouse TRBC2 | | D1 | GrGrArCrArArGrUrGrGr |
| | | | CrCrArGrArG/AltR2/ |
| MG29-1 | 5230 | MG29-1- | /AltR1/rUrArArUrUrUr |
| sgRNA | | mTRBC2- | CrUrArCrUrGrUrUrGrUr |
| targeting | | sgRNA- | ArGrArUrCrUrCrArUrGr |
| mouse TRBC2 | | E1 | ArGrCrUrCrCrGrCrArCr |
| | | | UrUrArCrCrU/AltR2/ |
| MG29-1 | 5231 | MG29-1- | /AltR1/rUrArArUrUrUr |
| sgRNA | | mTRBC2- | CrUrArCrUrGrUrUrGrUr |
| targeting | | sgRNA- | ArGrArUrGrGrUrGrArGr |
| mouse TRBC2 | | F1 | CrCrCrUrCrUrGrGrCrCr |
| | | | ArCrUrUrGrU/AltR2/ |
| MG29-1 | 5232 | MG29-1- | /AltR1/rUrArArUrUrUr |
| sgRNA | | mTRBC2- | CrUrArCrUrGrUrUrGrUr |
| targeting | | sgRNA- | ArGrArUrGrArGrGrArUr |
| mouse TRBC2 | | G1 | UrGrUrGrCrCrArGrArAr |
| | | | GrGrUrArGrC/AltR2/ |
| MG29-1 | 5233 | MG29-1- | /AltR1/rUrArArUrUrUr |
| sgRNA | | mTRBC2- | CrUrArCrUrGrUrUrGrUr |
| targeting | | sgRNA- | ArGrArUrGrUrUrUrGrUr |
| mouse TRBC2 | | H1 | UrUrGrCrArArUrCrUrCr |
| | | | UrGrCrUrUrU/AltR2/ |
| MG29-1 | 5234 | MG29-1- | /AltR1/rUrArArUrUrUr |
| sgRNA | | mTRBC2- | CrUrArCrUrGrUrUrGrUr |
| targeting | | sgRNA- | ArGrArUrUrUrUrGrUrUr |
| mouse TRBC2 | | A2 | UrGrCrArArUrCrUrCrUr |
| | | | GrCrUrUrUrU/AltR2/ |
| MG29-1 | 5235 | MG29-1- | /AltR1/rUrArArUrUrUr |
| sgRNA | | mTRBC2- | CrUrArCrUrGrUrUrGrUr |
| targeting | | sgRNA- | ArGrArUrUrUrUrGrCrAr |
| mouse TRBC2 | | B2 | ArUrCrUrCrUrGrCrUrUr |
| | | | UrUrGrArUrG/AltR2/ |
| MG29-1 | 5236 | MG29-1- | /AltR1/rUrArArUrUrUr |
| sgRNA | | mTRBC2- | CrUrArCrUrGrUrUrGrUr |
| targeting | | sgRNA- | ArGrArUrCrArArUrCrUr |
| mouse TRBC2 | | C2 | CrUrGrCrUrUrUrUrGrAr |
| | | | UrGrGrCrUrC/AltR2/ |
| MG29-1 | 5237 | MG29-1- | /AltR1/rUrArArUrUrUr |
| sgRNA | | mTRBC2- | CrUrArCrUrGrUrUrGrUr |
| targeting | | sgRNA- | ArGrArUrGrArUrGrGrCr |
| mouse TRBC2 | | D2 | UrCrArArArCrArArGrGr |
| | | | ArGrArCrCrU/AltR2/ |
| MG29-1 | 5238 | MG29-1- | /AltR1/rUrArArUrUrUr |
| sgRNA | | mTRBC2- | CrUrArCrUrGrUrUrGrUr |
| targeting | | sgRNA- | ArGrArUrArUrGrGrCrUr |
| mouse TRBC2 | | E2 | CrArArArCrArArGrGrAr |
| | | | GrArCrCrUrU/AltR2/ |
| MG29-1 | 5239 | MG29-1- | /AltR1/rUrArArUrUrUr |
| sgRNA | | mTRBC2- | CrUrArCrUrGrUrUrGrUr |
| targeting | | sgRNA- | ArGrArUrCrUrCrCrCrUr |
| mouse TRBC2 | | F2 | CrUrCrCrUrUrUrCrUrUr |
| | | | UrCrArGrArC/AltR2/ |
| MG29-1 | 5240 | MG29-1- | /AltR1/rUrArArUrUrUr |
| sgRNA | | mTRBC2- | CrUrArCrUrGrUrUrGrUr |
| targeting | | sgRNA- | ArGrArUrUrCrCrCrUrCr |
| mouse TRBC2 | | G2 | UrCrCrUrUrUrCrUrUrUr |
| | | | CrArGrArCrU/AltR2/ |
| MG29-1 | 5241 | MG29-1- | /AltR1/rUrArArUrUrUr |
| sgRNA | | mTRBC2- | CrUrArCrUrGrUrUrGrUr |
| targeting | | sgRNA- | ArGrArUrUrUrUrCrArGr |
| mouse TRBC2 | | H2 | ArCrUrGrUrGrGrArArUr |
| | | | CrArCrUrUrC/AltR2/ |
| MG29-1 | 5242 | MG29-1- | /AltR1/rUrArArUrUrUr |
| sgRNA | | mTRBC2- | CrUrArCrUrGrUrUrGrUr |
| targeting | | sgRNA- | ArGrArUrArGrArCrUrGr |
| mouse TRBC2 | | A3 | UrGrGrArArUrCrArCrUr |
| | | | UrCrArGrGrU/AltR2/ |
| MG29-1 | 5243 | MG29-1- | /AltR1/rUrArArUrUrUr |
| sgRNA | | mTRBC2- | CrUrArCrUrGrUrUrGrUr |
| targeting | | sgRNA- | ArGrArUrCrUrGrUrCrAr |
| mouse TRBC2 | | B3 | ArCrArGrCrArUrCrCrUr |
| | | | ArUrCrArUrC/AltR2/ |
| MG29-1 | 5244 | MG29-1- | /AltR1/rUrArArUrUrUr |
| sgRNA | | mTRBC2- | CrUrArCrUrGrUrUrGrUr |
| targeting | | sgRNA- | ArGrArUrUrCrCrArUrCr |
| mouse TRBC2 | | C3 | CrUrArCrCrArUrUrCrUr |
| | | | UrArCrCrArU/AltR2/ |
| MG29-1 | 5245 | MG29-1- | /AltR1/rUrArArUrUrUr |
| sgRNA | | mTRBC2- | CrUrArCrUrGrUrUrGrUr |
| targeting | | sgRNA- | ArGrArUrUrCrArGrGrUr |
| mouse TRBC2 | | D3 | CrArArGrArArArArArAr |
| | | | ArArUrUrCrC/AltR2/ |
| MG29-1 | 5246 | MG29-1- | /AltR1/rUrArArUrUrUr |
| sgRNA | | mTRBC2- | CrUrArCrUrGrUrUrGrUr |
| targeting | | sgRNA- | ArGrArUrUrCrUrCrArGr |
| mouse TRBC2 | | E3 | GrArArUrUrUrUrUrUrUr |
| | | | UrCrUrUrGrA/AltR2/ |
| DNA sequence | 5247 | MG29-1- | CTTTCCAGAGGATCTGAGAA |
| of TRBC2 | | mTRBC2- | AT |
| target site | | target | |
| | | site-A1 | |
| DNA sequence | 5248 | MG29-1- | CAGAGGATCTGAGAAATGTG |
| of TRBC2 | | mTRBC2- | AC |
| target site | | target | |
| | | site-B1 | |
| DNA sequence | 5249 | MG29-1- | AGCCATCAAAAGCAGAGATT |
| of TRBC2 | | mTRBC2- | GC |
| target site | | target | |
| | | site-C1 | |
| DNA sequence | 5250 | MG29-1- | AGAGGAGGACAAGTGGCCAG |
| of TRBC2 | | mTRBC2- | AG |
| target site | | target | |
| | | site-D1 | |
| DNA sequence | 5251 | MG29-1- | CTCATGAGCTCCGCACTTAC |
| of TRBC2 | | mTRBC2- | CT |
| target site | | target | |
| | | site-E1 | |
| DNA sequence | 5252 | MG29-1- | GGTGAGCCCTCTGGCCACTT |
| of TRBC2 | | mTRBC2- | GT |
| target site | | target | |
| | | site-F1 | |
| DNA sequence | 5253 | MG29-1- | GAGGATTGTGCCAGAAGGTA |
| of TRBC2 | | mTRBC2- | GC |
| target site | | target | |
| | | site-G1 | |
| DNA sequence | 5254 | MG29-1- | GTTTGTTTGCAATCTCTGCT |
| of TRBC2 | | mTRBC2- | TT |
| target site | | target | |
| | | site-H1 | |
| DNA sequence | 5255 | MG29-1- | TTTGTTTGCAATCTCTGCTT |
| of TRBC2 | | mTRBC2- | TT |
| target site | | target | |
| | | site-A2 | |
| DNA sequence | 5256 | MG29-1- | TTTGCAATCTCTGCTTTTGA |
| of TRBC2 | | mTRBC2- | TG |
| target site | | target | |
| | | site-B2 | |
| DNA sequence | 5257 | MG29-1- | CAATCTCTGCTTTTGATGGC |
| of TRBC2 | | mTRBC2- | TC |
| target site | | target | |
| | | site-C2 | |
| DNA sequence | 5258 | MG29-1- | GATGGCTCAAACAAGGAGAC |
| of TRBC2 | | mTRBC2- | CT |
| target site | | target | |
| | | site-D2 | |
| DNA sequence | 5259 | MG29-1- | ATGGCTCAAACAAGGAGACC |
| of TRBC2 | | mTRBC2- | TT |
| target site | | target | |
| | | site-E2 | |
| DNA sequence | 5260 | MG29-1- | CTCCCTCTCCTTTCTTTCAG |
| of TRBC2 | | mTRBC2- | AC |
| target site | | target | |
| | | site-F2 | |
| DNA sequence | 5261 | MG29-1- | TCCCTCTCCTTTCTTTCAGA |
| of TRBC2 | | mTRBC2- | CT |
| target site | | target | |
| | | site-G2 | |
| DNA sequence | 5262 | MG29-1- | TTTCAGACTGTGGAATCACT |
| of TRBC2 | | mTRBC2- | TC |
| target site | | target | |
| | | site-H2 | |
| DNA sequence | 5263 | MG29-1- | AGACTGTGGAATCACTTCAG |
| of TRBC2 | | mTRBC2- | GT |
| target site | | target | |
| | | site-A3 | |
| DNA sequence | 5264 | MG29-1- | CTGTCAACAGCATCCTATCA |
| of TRBC2 | | mTRBC2- | TC |
| target site | | target | |
| | | site-B3 | |
| DNA sequence | 5265 | MG29-1- | TCCATCCTACCATTCTTACC |
| of TRBC2 | | mTRBC2- | AT |
| target site | | target | |
| | | site-C3 | |
| DNA sequence | 5266 | MG29-1- | TCAGGTCAAGAAAAAAAATT |
| of TRBC2 | | mTRBC2- | CC |
| target site | | target | |
| | | site-D3 | |
| DNA sequence | 5267 | MG29-1- | TCTCAGGAATTTTTTTTCTT |
| of TRBC2 | | mTRBC2- | GA |
| target site | | target | |
| | | site-E3 | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 20—Gene Editing Outcomes at the DNA Level for Human TRBC1/2

Primary T cells were purified from PMBCs using a negative selection kit (Miltenyi) according to the manufacturer's recommendations. Nucleofection of MG29-1 RNPs (106 pmol protein/160 pmol guide) (SEQ ID Nos: 5642-5660) was performed into T cells (200,000) using the Lonza 4D electroporator. For analysis by flow cytometry, 3 days post-nucleofection, 100,000 T cells were stained with anti-CD3 antibody for 30 minutes at 4C and analyzed on an Attune Nxt flow cytometer (FIG. 66C).

TABLE 14I
Sequences of Guide RNAs and Sequences
Targeted for Example 46

| | SEQ | | |
|---|---|---|---|
| Guide | ID | Guide | |
| Target | NO | Name | SEQUENCE |
| MG29-1 | 5642 | MG29-1- | /AltR1/rUrArArUrUrUr |
| sgRNA | | TRBC1/2- | CrUrArCrUrGrUrUrGrUr |
| targeting | | sgRNA- | ArGrArUrArGrCrCrArU |
| human | | A1 | rCrArGrArArGrCrArGrA |
| TRBC1/2 | | | rGrArUrC/AltR2/ |
| MG29-1 | 5643 | MG29-1- | /AltR1/rUrArArUrUrUr |
| sgRNA | | TRBC1/2- | CrUrArCrUrGrUrUrGrUr |
| targeting | | sgRNA- | ArGrArUrGrGrUrGrUrG |
| human | | B1 | rGrGrArGrArUrCrUrCrU |
| TRBC1/2 | | | rGrCrUrU/AltR2/ |
| MG29-1 | 5644 | MG29-1- | /AltR1/rUrArArUrUrUr |
| sgRNA | | TRBC1/2- | CrUrArCrUrGrUrUrGrUr |
| targeting | | sgRNA- | ArGrArUrGrGrGrUrGrU |
| human | | C1 | rGrGrGrArGrArUrCrUrC |
| TRBC1/2 | | | rUrGrCrU/AltR2/ |
| MG29-1 | 5645 | MG29-1- | /AltR1/rUrArArUrUrUr |
| sgRNA | | TRBC1/2- | CrUrArCrUrGrUrUrGrUr |
| targeting | | sgRNA- | ArGrArUrGrCrCrCrUrA |
| human | | D1 | rUrCrCrUrGrGrGrUrCrC |
| TRBC1/2 | | | rArCrUrC/AltR2/ |
| MG29-1 | 5646 | MG29-1- | /AltR1/rUrArArUrUrUr |
| sgRNA | | TRBC1/2- | CrUrArCrUrGrUrUrGrUr |
| targeting | | sgRNA- | ArGrArUrUrUrUrCrArG |
| human | | E1 | rArCrUrGrUrGrGrCrUrU |
| TRBC1/2 | | | rUrArCrC/AltR2/ |
| MG29-1 | 5647 | MG29-1- | /AltR1/rUrArArUrUrUr |
| sgRNA | | TRBC1/2- | CrUrArCrUrGrUrUrGrUr |
| targeting | | sgRNA- | ArGrArUrArGrArCrUrG |
| human | | F1 | rUrGrGrCrUrUrUrArCrC |
| TRBC1/2 | | | rUrCrGrG/AltR2/ |
| MG29-1 | 5648 | MG29-1- | /AltR1/rUrArArUrUrUr |
| sgRNA | | TRBC1/2- | CrUrArCrUrGrUrUrGrUr |
| targeting | | sgRNA- | ArGrArUrUrCrUrUrCrU |
| human | | G1 | rGrCrArGrGrUrCrArArG |
| TRBC1/2 | | | rArGrArA/AltR2/ |
| MG29-1 | 5649 | MG29-1- | /AltR1/rUrArArUrUrUr |
| sgRNA | | TRBC1/2- | CrUrArCrUrGrUrUrGrUr |
| targeting | | sgRNA- | ArGrArUrUrCrUrUrGrA |
| human | | H1 | rCrCrUrGrCrArGrArArG |
| TRBC1/2 | | | rArGrArA/AltR2/ |
| MG29-1 | 5650 | MG29-1- | /AltR1/rUrArArUrUrUr |
| sgRNA | | TRBC1/2- | CrUrArCrUrGrUrUrGrUr |
| targeting | | sgRNA- | ArGrArUrArGrGrUrCrC |
| human | | A2 | rUrCrUrGrGrArArArGrG |
| TRBC1/2 | | | rGrArArG/AltR2/ |
| MG29-1 | 5651 | MG29-1- | /AltR1/rUrArArUrUrUr |
| sgRNA | | TRBC1/2- | CrUrArCrUrGrUrUrGrUr |
| targeting | | sgRNA- | ArGrArUrCrArGrGrUrC |
| human | | B2 | rCrUrCrUrGrGrArArArG |
| TRBC1/2 | | | rGrGrArA/AltR2/ |
| MG29-1 | 5652 | MG29-1- | /AltR1/rUrArArUrUrUr |
| sgRNA | | TRBC1/2- | CrUrArCrUrGrUrUrGrUr |
| targeting | | sgRNA- | ArGrArUrUrCrArGrGrU |
| human | | C2 | rCrCrUrCrUrGrGrArArA |
| TRBC1/2 | | | rGrGrGrA/AltR2/ |
| MG29-1 | 5653 | MG29-1- | /AltR1/rUrArArUrUrUr |
| sgRNA | | TRBC1/2- | CrUrArCrUrGrUrUrGrUr |
| targeting | | sgRNA- | ArGrArUrArGrCrCrArU |
| human | | D2 | rCrArGrArArGrCrArGrA |
| TRBC1/2 | | | rGrArUrC/AltR2/ |
| MG29-1 | 5654 | MG29-1- | /AltR1/rUrArArUrUrUr |
| sgRNA | | TRBC1/2- | CrUrArCrUrGrUrUrGrUr |
| targeting | | sgRNA- | ArGrArUrGrGrUrGrUrG |
| human | | E2 | rGrGrArGrArUrCrUrCrU |
| TRBC1/2 | | | rGrCrUrU/AltR2/ |
| MG29-1 | 5655 | MG29-1- | /AltR1/rUrArArUrUrUr |
| sgRNA | | TRBC1/2- | CrUrArCrUrGrUrUrGrUr |
| targeting | | sgRNA- | ArGrArUrGrGrGrUrGrU |
| human | | F2 | rGrGrGrArGrArUrCrUrC |
| TRBC1/2 | | | rUrGrCrU/AltR2/ |
| MG29-1 | 5656 | MG29-1- | /AltR1/rUrArArUrUrUr |
| sgRNA | | TRBC1/2- | CrUrArCrUrGrUrUrGrUr |
| targeting | | sgRNA- | ArGrArUrGrCrCrCrUrA |
| human | | G2 | rUrCrCrUrGrGrGrUrCrC |
| TRBC1/2 | | | rArCrUrC/AltR2/ |
| MG29-1 | 5657 | MG29-1- | /AltR1/rUrArArUrUrUr |
| sgRNA | | TRBC1/2- | CrUrArCrUrGrUrUrGrUr |
| targeting | | sgRNA- | ArGrArUrCrUrUrUrCrA |
| human | | H2 | rGrArCrUrGrUrGrGrCrU |
| TRBC1/2 | | | rUrCrArC/AltR2/ |
| MG29-1 | 5658 | MG29-1- | /AltR1/rUrArArUrUrUr |
| sgRNA | | TRBC1/2- | CrUrArCrUrGrUrUrGrUr |
| targeting | | sgRNA- | ArGrArUrUrUrUrCrArG |
| human | | A3 | rArCrUrGrUrGrGrCrUrU |
| TRBC1/2 | | | rCrArCrC/AltR2/ |
| MG29-1 | 5659 | MG29-1- | /AltR1/rUrArArUrUrUr |
| sgRNA | | TRBC1/2- | CrUrArCrUrGrUrUrGrUr |
| targeting | | sgRNA- | ArGrArUrArGrArCrUrG |
| human | | B3 | rUrGrGrCrUrUrCrArCrC |
| TRBC1/2 | | | rUrCrCrG/AltR2/ |
| MG29-1 | 5660 | MG29-1- | /AltR1/rUrArArUrUrUr |
| sgRNA | | TRBC1/2- | CrUrArCrUrGrUrUrGrUr |
| targeting | | sgRNA- | ArGrArUrUrCrUrUrGrA |
| human | | C3 | rCrCrUrGrUrGrGrArArG |
| TRBC1/2 | | | rArGrArG/AltR2/ |
| DNA sequence | 5661 | MG29-1- | AGCCATCAGAAGCAGAGATC |
| of TRBC1/2 | | TRBC1/2- | |
| target site | | target | |
| | | site-A1 | |
| DNA sequence | 5662 | MG29-1- | GGTGTGGGAGATCTCTGCTT |
| of TRBC1/2 | | TRBC1/2- | |
| target site | | target | |
| | | site-B1 | |
| DNA sequence | 5663 | MG29-1- | GGGTGTGGGAGATCTCTGCT |
| of TRBC1/2 | | TRBC1/2- | |
| target site | | target | |
| | | site-C1 | |
| DNA sequence | 5664 | MG29-1- | GCCCTATCCTGGGTCCACTC |
| of TRBC1/2 | | TRBC1/2- | |
| target site | | target | |
| | | site-D1 | |
| DNA sequence | 5665 | MG29-1- | TTTCAGACTGTGGCTTTACC |
| of TRBC1/2 | | TRBC1/2- | |
| target site | | target | |
| | | site-E1 | |
| DNA sequence | 5666 | MG29-1- | AGACTGTGGCTTTACCTCGG |
| of TRBC1/2 | | TRBC1/2- | |
| target site | | target | |
| | | site-F1 | |
| DNA sequence | 5667 | MG29-1- | TCTTCTGCAGGTCAAGAGAA |
| of TRBC1/2 | | TRBC1/2- | |
| target site | | target | |
| | | site-G1 | |
| DNA sequence | 5668 | MG29-1- | TCTTGACCTGCAGAAGAGAA |
| of TRBC1/2 | | TRBC1/2- | |
| target site | | target | |
| | | site-H1 | |
| DNA sequence | 5669 | MG29-1- | AGGTCCTCTGGAAAGGGAAG |
| of TRBC1/2 | | TRBC1/2- | |
| target site | | target | |
| | | site-A2 | |
| DNA sequence | 5670 | MG29-1- | CAGGTCCTCTGGAAAGGGAA |
| of TRBC1/2 | | TRBC1/2- | |
| target site | | target | |
| | | site-B2 | |
| DNA sequence | 5671 | MG29-1- | TCAGGTCCTCTGGAAAGGGA |
| of TRBC1/2 | | TRBC1/2- | |
| target site | | target | |
| | | site-C2 | |
| DNA sequence | 5672 | MG29-1- | AGCCATCAGAAGCAGAGATC |
| of TRBC1/2 | | TRBC1/2- | |
| target site | | target | |
| | | site-D2 | |
| DNA sequence | 5673 | MG29-1- | GGTGTGGGAGATCTCTGCTT |
| of TRBC1/2 | | TRBC1/2- | |
| target site | | target | |
| | | site-E2 | |
| DNA sequence | 5674 | MG29-1- | GGGTGTGGGAGATCTCTGCT |
| of TRBC1/2 | | TRBC1/2- | |
| target site | | target | |
| | | site-F2 | |
| DNA sequence | 5675 | MG29-1- | GCCCTATCCTGGGTCCACTC |
| of TRBC1/2 | | TRBC1/2- | |
| target site | | target | |
| | | site-G2 | |
| DNA sequence | 5676 | MG29-1- | CTTTCAGACTGTGGCTTCAC |
| of TRBC1/2 | | TRBC1/2- | |
| target site | | target | |
| | | site-H2 | |
| DNA sequence | 5677 | MG29-1- | TTTCAGACTGTGGCTTCACC |
| of TRBC1/2 | | TRBC1/2- | |
| target site | | target | |
| | | site-A3 | |
| DNA sequence | 5678 | MG29-1- | AGACTGTGGCTTCACCTCCG |
| of TRBC1/2 | | TRBC1/2- | |
| target site | | target | |
| | | site-B3 | |
| DNA sequence | 5679 | MG29-1- | TCTTGACCTGTGGAAGAGAG |
| of TRBC1/2 | | TRBC1/2- | |
| target site | | target | |
| | | site-C3 | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 21—Gene Editing Outcomes at the DNA Level for HPRT

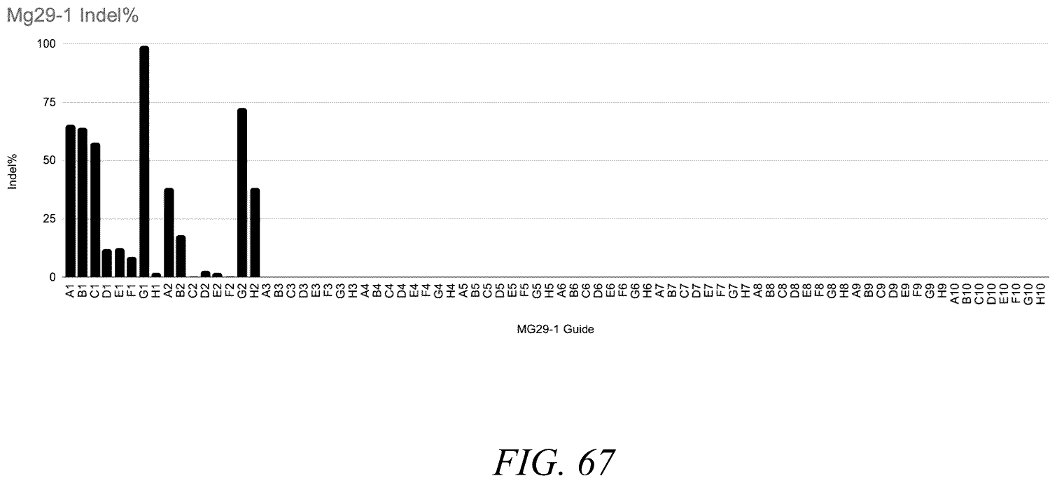

Primary T cells were purified from PMBCs using a negative selection kit (Miltenyi) according to the manufacturer's recommendations. Nucleofection of MG29-1 RNPs (126 pmol protein/160 pmol guide) (SEQ ID NOs: 5482-5561) was performed into T cells (200,000) using the Lonza 4D electroporator. Cells were harvested and genomic DNA prepared five days post-transfection. PCR primers appropriate for use in NGS-based DNA sequencing were generated, optimized, and used to amplify the individual target sequences for each guide RNA (SEQ ID NOs: 5562-5641). The amplicons were sequenced on an Illumina MiSeq machine and analyzed with a proprietary Python script to measure gene editing (FIG. 67).

TABLE 14J
Sequences of Guide RNAs and Sequences
Targeted for Example 47

| | SEQ | | |
|---|---|---|---|
| Guide | ID | Guide | |
| Target | NO | Name | SEQUENCE |
| MG29-1 | 5482 | MG29-1- | /AltR1/rUrArArUrUrUr |
| sgRNA | | HPRT- | CrUrArCrUrGrUrUrGrUr |
| targeting | | sgRNA-A1 | ArGrArUrUrCrUrCrCrCr |
| HPRT | | | UrGrGrCrUrUrArCrCrUr |
| | | | UrUrArGrGrA/AltR2/ |
| MG29-1 | 5483 | MG29-1- | /AltR1/rUrArArUrUrUr |
| sgRNA | | HPRT- | CrUrArCrUrGrUrUrGrUr |
| targeting | | sgRNA-B1 | ArGrArUrGrArArCrArCr |
| HPRT | | | ArArGrCrCrCrArCrCrAr |
| | | | UrUrArArArA/AltR2/ |
| MG29-1 | 5484 | MG29-1- | /AltR1/rUrArArUrUrUr |
| sgRNA | | HPRT- | CrUrArCrUrGrUrUrGrUr |
| targeting | | sgRNA-C1 | ArGrArUrArArUrGrGrUr |
| HPRT | | | GrGrGrCrUrUrGrUrGrUr |
| | | | UrCrUrArArA/AltR2/ |
| MG29-1 | 5485 | MG29-1- | /AltR1/rUrArArUrUrUr |
| sgRNA | | HPRT- | CrUrArCrUrGrUrUrGrUr |
| targeting | | sgRNA-D1 | ArGrArUrArUrGrGrUrGr |
| HPRT | | | GrGrCrUrUrGrUrGrUrUr |
| | | | CrUrArArArG/AltR2/ |
| MG29-1 | 5486 | MG29-1- | /AltR1/rUrArArUrUrUr |
| sgRNA | | HPRT- | CrUrArCrUrGrUrUrGrUr |
| targeting | | sgRNA-E1 | ArGrArUrCrCrCrUrArAr |
| HPRT | | | CrArArArGrArUrGrGrGr |
| | | | UrUrUrGrUrU/AltR2/ |
| MG29-1 | 5487 | MG29-1- | /AltR1/rUrArArUrUrUr |
| sgRNA | | HPRT- | CrUrArCrUrGrUrUrGrUr |
| targeting | | sgRNA-F1 | ArGrArUrArArGrGrCrAr |
| HPRT | | | CrCrCrArArArUrUrArAr |
| | | | UrArArCrGrC/AltR2/ |
| MG29-1 | 5488 | MG29-1- | /AltR1/rUrArArUrUrUr |
| sgRNA | | HPRT- | CrUrArCrUrGrUrUrGrUr |
| targeting | | sgRNA-G1 | ArGrArUrUrArGrCrGrUr |
| HPRT | | | UrArUrUrArArUrUrUrGr |
| | | | GrGrUrGrCrC/AltR2/ |
| MG29-1 | 5489 | MG29-1- | /AltR1/rUrArArUrUrUr |
| sgRNA | | HPRT- | CrUrArCrUrGrUrUrGrUr |
| targeting | | sgRNA-H1 | ArGrArUrUrCrGrArGrUr |
| HPRT | | | GrUrArGrUrCrUrGrUrUr |
| | | | ArGrCrCrArC/AltR2/ |
| MG29-1 | 5490 | MG29-1- | /AltR1/rUrArArUrUrUr |
| sgRNA | | HPRT- | CrUrArCrUrGrUrUrGrUr |
| targeting | | sgRNA-A2 | ArGrArUrUrUrArGrGrGr |
| HPRT | | | ArGrUrUrArUrGrArUrGr |
| | | | UrUrGrUrCrC/AltR2/ |
| MG29-1 | 5491 | MG29-1- | /AltR1/rUrArArUrUrUr |
| sgRNA | | HPRT- | CrUrArCrUrGrUrUrGrUr |
| targeting | | sgRNA-B2 | ArGrArUrUrArGrGrUrGr |
| HPRT | | | CrArGrGrArUrCrArArUr |
| | | | GrArCrArGrC/AltR2/ |
| MG29-1 | 5492 | MG29-1- | /AltR1/rUrArArUrUrUr |
| sgRNA | | HPRT- | CrUrArCrUrGrUrUrGrUr |
| targeting | | sgRNA-C2 | ArGrArUrGrUrArGrGrUr |
| HPRT | | | GrCrArGrGrArUrCrArAr |
| | | | UrGrArCrArG/AltR2/ |
| MG29-1 | 5493 | MG29-1- | /AltR1/rUrArArUrUrUr |
| sgRNA | | HPRT- | CrUrArCrUrGrUrUrGrUr |
| targeting | | sgRNA-D2 | ArGrArUrCrUrGrUrCrAr |
| HPRT | | | UrUrGrArUrCrCrUrGrCr |
| | | | ArCrCrUrArC/AltR2/ |
| MG29-1 | 5494 | MG29-1- | /AltR1/rUrArArUrUrUr |
| sgRNA | | HPRT- | CrUrArCrUrGrUrUrGrUr |
| targeting | | sgRNA-E2 | ArGrArUrCrArGrCrArCr |
| HPRT | | | ArGrUrArArUrUrCrUrCr |
| | | | ArCrCrArArA/AltR2/ |
| MG29-1 | 5495 | MG29-1- | /AltR1/rUrArArUrUrUr |
| sgRNA | | HPRT- | CrUrArCrUrGrUrUrGrUr |
| targeting | | sgRNA-F2 | ArGrArUrGrGrUrGrArGr |
| HPRT | | | ArArUrUrArCrUrGrUrGr |
| | | | CrUrGrArArA/AltR2/ |
| MG29-1 | 5496 | MG29-1- | /AltR1/rUrArArUrUrUr |
| sgRNA | | HPRT- | CrUrArCrUrGrUrUrGrUr |
| targeting | | sgRNA-G2 | ArGrArUrUrCrCrUrGrAr |
| HPRT | | | ArUrArGrCrArUrGrGrCr |
| | | | ArGrArGrGrA/AltR2/ |
| MG29-1 | 5497 | MG29-1- | /AltR1/rUrArArUrUrUr |
| sgRNA | | HPRT- | CrUrArCrUrGrUrUrGrUr |
| targeting | | sgRNA-H2 | ArGrArUrCrArArGrGrGr |
| HPRT | | | GrGrCrCrCrArArArArUr |
| | | | CrCrUrCrUrG/AltR2/ |
| MG29-1 | 5498 | MG29-1- | /AltR1/rUrArArUrUrUr |
| sgRNA | | HPRT- | CrUrArCrUrGrUrUrGrUr |
| targeting | | sgRNA-A3 | ArGrArUrGrCrArArGrGr |
| HPRT | | | GrGrGrCrCrCrArArArAr |
| | | | UrCrCrUrCrU/AltR2/ |
| MG29-1 | 5499 | MG29-1- | /AltR1/rUrArArUrUrUr |
| sgRNA | | HPRT- | CrUrArCrUrGrUrUrGrUr |
| targeting | | sgRNA-B3 | ArGrArUrGrGrGrCrCrCr |
| HPRT | | | CrCrUrUrGrCrArArArAr |
| | | | UrUrArArGrA/AltR2/ |
| MG29-1 | 5500 | MG29-1- | /AltR1/rUrArArUrUrUr |
| sgRNA | | HPRT- | CrUrArCrUrGrUrUrGrUr |
| targeting | | sgRNA-C3 | ArGrArUrGrGrCrCrCrCr |
| HPRT | | | CrUrUrGrCrArArArArUr |
| | | | UrArArGrArA/AltR2/ |
| MG29-1 | 5501 | MG29-1- | /AltR1/rUrArArUrUrUr |
| sgRNA | | HPRT- | CrUrArCrUrGrUrUrGrUr |
| targeting | | sgRNA-D3 | ArGrArUrArCrUrArCrAr |
| HPRT | | | GrArCrArCrArArArGrAr |
| | | | ArGrArUrGrC/AltR2/ |
| MG29-1 | 5502 | MG29-1- | /AltR1/rUrArArUrUrUr |
| sgRNA | | HPRT- | CrUrArCrUrGrUrUrGrUr |
| targeting | | sgRNA-E3 | ArGrArUrUrUrArUrUrAr |
| HPRT | | | ArGrUrCrGrGrCrCrUrCr |
| | | | ArCrCrUrCrC/AltR2/ |
| MG29-1 | 5503 | MG29-1- | /AltR1/rUrArArUrUrUr |
| sgRNA | | HPRT- | CrUrArCrUrGrUrUrGrUr |
| targeting | | sgRNA-F3 | ArGrArUrUrArUrUrArAr |
| HPRT | | | GrUrCrGrGrCrCrUrCrAr |
| | | | CrCrUrCrCrU/AltR2/ |
| MG29-1 | 5504 | MG29-1- | /AltR1/rUrArArUrUrUr |
| sgRNA | | HPRT- | CrUrArCrUrGrUrUrGrUr |
| targeting | | sgRNA-G3 | ArGrArUrArUrUrArArGr |
| HPRT | | | UrCrGrGrCrCrUrCrArCr |
| | | | CrUrCrCrUrC/AltR2/ |
| MG29-1 | 5505 | MG29-1- | /AltR1/rUrArArUrUrUr |
| sgRNA | | HPRT- | CrUrArCrUrGrUrUrGrUr |
| targeting | | sgRNA-H3 | ArGrArUrUrUrArArGrUr |
| HPRT | | | CrGrGrCrCrUrCrArCrCr |
| | | | UrCrCrUrCrA/AltR2/ |
| MG29-1 | 5506 | MG29-1- | /AltR1/rUrArArUrUrUr |
| sgRNA | | HPRT- | CrUrArCrUrGrUrUrGrUr |
| targeting | | sgRNA-A4 | ArGrArUrUrArUrGrUrAr |
| HPRT | | | GrGrGrGrUrCrArGrGrUr |
| | | | ArArUrGrUrU/AltR2/ |
| MG29-1 | 5507 | MG29-1- | /AltR1/rUrArArUrUrUr |
| sgRNA | | HPRT- | CrUrArCrUrGrUrUrGrUr |
| targeting | | sgRNA-B4 | ArGrArUrArUrGrUrArGr |
| HPRT | | | GrGrGrUrCrArGrGrUrAr |
| | | | ArUrGrUrUrC/AltR2/ |
| MG29-1 | 5508 | MG29-1- | /AltR1/rUrArArUrUrUr |
| sgRNA | | HPRT- | CrUrArCrUrGrUrUrGrUr |
| targeting | | sgRNA-C4 | ArGrArUrUrGrUrArGrGr |
| HPRT | | | GrGrUrCrArGrGrUrArAr |
| | | | UrGrUrUrCrU/AltR2/ |
| MG29-1 | 5509 | MG29-1- | /AltR1/rUrArArUrUrUr |
| sgRNA | | HPRT- | CrUrArCrUrGrUrUrGrUr |
| targeting | | sgRNA-D4 | ArGrArUrGrArArArArAr |
| HPRT | | | ArUrCrArCrGrGrUrArUr |
| | | | CrUrGrUrCrG/AltR2/ |
| MG29-1 | 5510 | MG29-1- | /AltR1/rUrArArUrUrUr |
| sgRNA | | HPRT- | CrUrArCrUrGrUrUrGrUr |
| targeting | | sgRNA-E4 | ArGrArUrArArUrCrArGr |
| HPRT | | | ArGrUrArArGrCrCrUrUr |
| | | | CrUrArGrUrG/AltR2/ |
| MG29-1 | 5511 | MG29-1- | /AltR1/rUrArArUrUrUr |
| sgRNA | | HPRT- | CrUrArCrUrGrUrUrGrUr |
| targeting | | sgRNA-F4 | ArGrArUrGrGrArCrArGr |
| HPRT | | | GrUrArCrUrArUrGrArGr |
| | | | ArGrUrArUrA/AltR2/ |
| MG29-1 | 5512 | MG29-1- | /AltR1/rUrArArUrUrUr |
| sgRNA | | HPRT- | CrUrArCrUrGrUrUrGrUr |
| targeting | | sgRNA-G4 | ArGrArUrArArArUrGrUr |
| HPRT | | | CrArArCrCrUrArCrUrGr |
| | | | UrGrGrCrArU/AltR2/ |
| MG29-1 | 5513 | MG29-1- | /AltR1/rUrArArUrUrUr |
| sgRNA | | HPRT- | CrUrArCrUrGrUrUrGrUr |
| targeting | | sgRNA-H4 | ArGrArUrArArUrGrUrCr |
| HPRT | | | ArArCrCrUrArCrUrGrUr |
| | | | GrGrCrArUrA/AltR2/ |
| MG29-1 | 5514 | MG29-1- | /AltR1/rUrArArUrUrUr |
| sgRNA | | HPRT- | CrUrArCrUrGrUrUrGrUr |
| targeting | | sgRNA-A5 | ArGrArUrGrUrUrGrUrUr |
| HPRT | | | CrUrUrUrCrCrUrGrGrUr |
| | | | ArUrArUrGrC/AltR2/ |
| MG29-1 | 5515 | MG29-1- | /AltR1/rUrArArUrUrUr |
| sgRNA | | HPRT- | CrUrArCrUrGrUrUrGrUr |
| targeting | | sgRNA-B5 | ArGrArUrCrUrGrGrUrAr |
| HPRT | | | UrArUrGrCrUrGrUrGrGr |
| | | | ArArUrUrGrA/AltR2/ |
| MG29-1 | 5516 | MG29-1- | /AltR1/rUrArArUrUrUr |
| sgRNA | | HPRT- | CrUrArCrUrGrUrUrGrUr |
| targeting | | sgRNA-C5 | ArGrArUrArGrCrArUrGr |
| HPRT | | | UrCrCrUrArCrCrUrGrUr |
| | | | GrGrCrArArC/AltR2/ |
| MG29-1 | 5517 | MG29-1- | /AltR1/rUrArArUrUrUr |
| sgRNA | | HPRT- | CrUrArCrUrGrUrUrGrUr |
| targeting | | sgRNA-D5 | ArGrArUrGrUrGrUrUrGr |
| HPRT | | | CrCrArCrArGrGrUrArGr |
| | | | GrArCrArUrG/AltR2/ |
| MG29-1 | 5518 | MG29-1- | /AltR1/rUrArArUrUrUr |
| sgRNA | | HPRT- | CrUrArCrUrGrUrUrGrUr |
| targeting | | sgRNA-E5 | ArGrArUrUrGrUrUrGrCr |
| HPRT | | | CrArCrArGrGrUrArGrGr |
| | | | ArCrArUrGrC/AltR2/ |
| MG29-1 | 5519 | MG29-1- | /AltR1/rUrArArUrUrUr |
| sgRNA | | HPRT- | CrUrArCrUrGrUrUrGrUr |
| targeting | | sgRNA-F5 | ArGrArUrCrArUrArCrUr |
| HPRT | | | GrUrArArArUrGrGrGrUr |
| | | | ArArCrCrGrU/AltR2/ |
| MG29-1 | 5520 | MG29-1- | /AltR1/rUrArArUrUrUr |
| sgRNA | | HPRT- | CrUrArCrUrGrUrUrGrUr |
| targeting | | sgRNA-G5 | ArGrArUrCrCrArUrArCr |
| HPRT | | | UrGrUrArArArUrGrGrGr |
| | | | UrArArCrCrG/AltR2/ |
| MG29-1 | 5521 | MG29-1- | /AltR1/rUrArArUrUrUr |
| sgRNA | | HPRT- | CrUrArCrUrGrUrUrGrUr |
| targeting | | sgRNA-H5 | ArGrArUrArUrArArArGr |
| HPRT | | | CrUrCrCrArUrCrUrCrUr |
| | | | ArArGrGrCrA/AltR2/ |
| MG29-1 | 5522 | MG29-1- | /AltR1/rUrArArUrUrUr |
| sgRNA | | HPRT- | CrUrArCrUrGrUrUrGrUr |
| targeting | | sgRNA-A6 | ArGrArUrGrUrGrArUrAr |
| HPRT | | | CrCrUrUrUrUrCrUrGrGr |
| | | | ArGrCrArUrU/AltR2/ |
| MG29-1 | 5523 | MG29-1- | /AltR1/rUrArArUrUrUr |
| sgRNA | | HPRT- | CrUrArCrUrGrUrUrGrUr |
| targeting | | sgRNA-B6 | ArGrArUrCrUrGrGrArGr |
| HPRT | | | CrArUrUrCrCrUrGrArGr |
| | | | UrUrCrArGrG/AltR2/ |
| MG29-1 | 5524 | MG29-1- | /AltR1/rUrArArUrUrUr |
| sgRNA | | HPRT- | CrUrArCrUrGrUrUrGrUr |
| targeting | | sgRNA-C6 | ArGrArUrUrGrGrArGrCr |
| HPRT | | | ArUrUrCrCrUrGrArGrUr |
| | | | UrCrArGrGrU/AltR2/ |
| MG29-1 | 5525 | MG29-1- | /AltR1/rUrArArUrUrUr |
| sgRNA | | HPRT- | CrUrArCrUrGrUrUrGrUr |
| targeting | | sgRNA-D6 | ArGrArUrUrGrCrUrGrUr |
| HPRT | | | GrArUrUrGrGrCrUrUrGr |
| | | | UrUrArUrGrU/AltR2 |
| MG29-1 | 5526 | MG29-1- | /AltR1/rUrArArUrUrUr |
| sgRNA | | HPRT- | CrUrArCrUrGrUrUrGrUr |
| targeting | | sgRNA-E6 | ArGrArUrGrCrUrGrUrGr |
| HPRT | | | ArUrUrGrGrCrUrUrGrUr |
| | | | UrArUrGrUrU/AltR2/ |
| MG29-1 | 5527 | MG29-1- | /AltR1/rUrArArUrUrUr |
| sgRNA | | HPRT- | CrUrArCrUrGrUrUrGrUr |
| targeting | | sgRNA-F6 | ArGrArUrCrUrGrUrGrAr |
| HPRT | | | UrUrGrGrCrUrUrGrUrUr |
| | | | ArUrGrUrUrC/AltR2/ |
| MG29-1 | 5528 | MG29-1- | /AltR1/rUrArArUrUrUr |
| sgRNA | | HPRT- | CrUrArCrUrGrUrUrGrUr |
| targeting | | sgRNA-G6 | ArGrArUrUrUrGrGrArAr |
| HPRT | | | GrArGrUrCrArUrGrArGr |
| | | | GrGrArCrArU/AltR2/ |
| MG29-1 | 5529 | MG29-1- | /AltR1/rUrArArUrUrUr |
| sgRNA | | HPRT- | CrUrArCrUrGrUrUrGrUr |
| targeting | | sgRNA-H6 | ArGrArUrCrArGrArUrGr |
| HPRT | | | UrUrArArArGrGrCrArGr |
| | | | UrCrUrCrArA/AltR2/ |
| MG29-1 | 5530 | MG29-1- | /AltR1/rUrArArUrUrUr |
| sgRNA | | HPRT- | CrUrArCrUrGrUrUrGrUr |
| targeting | | sgRNA-A7 | ArGrArUrCrUrUrGrArGr |
| HPRT | | | ArCrUrGrCrCrUrUrUrAr |
| | | | ArCrArUrCrU/AltR2/ |
| MG29-1 | 5531 | MG29-1- | /AltR1/rUrArArUrUrUr |
| sgRNA | | HPRT- | CrUrArCrUrGrUrUrGrUr |
| targeting | | sgRNA-B7 | ArGrArUrUrUrGrArGrAr |
| HPRT | | | CrUrGrCrCrUrUrUrArAr |
| | | | CrArUrCrUrG/AltR2/ |
| MG29-1 | 5532 | MG29-1- | /AltR1/rUrArArUrUrUr |
| sgRNA | | HPRT- | CrUrArCrUrGrUrUrGrUr |
| targeting | | sgRNA-C7 | ArGrArUrUrArArCrCrCr |
| HPRT | | | ArArArUrGrCrUrGrCrCr |
| | | | UrGrUrUrGrA/AltR2/ |
| MG29-1 | 5533 | MG29-1- | /AltR1/rUrArArUrUrUr |
| sgRNA | | HPRT- | CrUrArCrUrGrUrUrGrUr |
| targeting | | sgRNA-D7 | ArGrArUrArUrArArCrCr |
| HPRT | | | CrArArArUrGrCrUrGrCr |
| | | | CrUrGrUrUrG/AltR2/ |
| MG29-1 | 5534 | MG29-1- | /AltR1/rUrArArUrUrUr |
| sgRNA | | HPRT- | CrUrArCrUrGrUrUrGrUr |
| targeting | | sgRNA-E7 | ArGrArUrUrCrArArCrAr |
| HPRT | | | GrGrCrArGrCrArUrUrUr |
| | | | GrGrGrUrUrA/AltR2/ |
| MG29-1 | 5535 | MG29-1- | /AltR1/rUrArArUrUrUr |
| sgRNA | | HPRT- | CrUrArCrUrGrUrUrGrUr |
| targeting | | sgRNA-F7 | ArGrArUrCrArArCrArGr |
| HPRT | | | GrCrArGrCrArUrUrUrGr |
| | | | GrGrUrUrArU/AltR2/ |
| MG29-1 | 5536 | MG29-1- | /AltR1/rUrArArUrUrUr |
| sgRNA | | HPRT- | CrUrArCrUrGrUrUrGrUr |
| targeting | | sgRNA-G7 | ArGrArUrArArCrArGrGr |
| HPRT | | | CrArGrCrArUrUrUrGrGr |
| | | | GrUrUrArUrA/AltR2/ |
| MG29-1 | 5537 | MG29-1- | /AltR1/rUrArArUrUrUr |
| sgRNA | | HPRT- | CrUrArCrUrGrUrUrGrUr |
| targeting | | sgRNA-H7 | ArGrArUrGrArArCrArAr |
| HPRT | | | ArArGrCrUrGrGrArGrGr |
| | | | UrGrGrUrArU/AltR2/ |
| MG29-1 | 5538 | MG29-1- | /AltR1/rUrArArUrUrUr |
| sgRNA | | HPRT- | CrUrArCrUrGrUrUrGrUr |
| targeting | | sgRNA-A8 | ArGrArUrGrGrUrArGrAr |
| HPRT | | | GrUrUrGrArCrUrUrArUr |
| | | | ArCrCrArCrC/AltR2/ |
| MG29-1 | 5539 | MG29-1- | /AltR1/rUrArArUrUrUr |
| sgRNA | | HPRT- | CrUrArCrUrGrUrUrGrUr |
| targeting | | sgRNA-B8 | ArGrArUrGrUrArGrArGr |
| HPRT | | | UrUrGrArCrUrUrArUrAr |
| | | | CrCrArCrCrU/AltR2/ |
| MG29-1 | 5540 | MG29-1- | /AltR1/rUrArArUrUrUr |
| sgRNA | | HPRT- | CrUrArCrUrGrUrUrGrUr |
| targeting | | sgRNA-C8 | ArGrArUrGrGrArArCrAr |
| HPRT | | | ArArArGrCrUrGrGrArGr |
| | | | GrUrGrGrUrA/AltR2/ |
| MG29-1 | 5541 | MG29-1- | /AltR1/rUrArArUrUrUr |
| sgRNA | | HPRT- | CrUrArCrUrGrUrUrGrUr |
| targeting | | sgRNA-D8 | ArGrArUrUrGrGrArArCr |
| HPRT | | | ArArArArGrCrUrGrGrAr |
| | | | GrGrUrGrGrU/AltR2/ |
| MG29-1 | 5542 | MG29-1- | /AltR1/rUrArArUrUrUr |
| sgRNA | | HPRT- | CrUrArCrUrGrUrUrGrUr |
| targeting | | sgRNA-E8 | ArGrArUrUrUrUrUrUrGr |
| HPRT | | | GrArArCrArArArArGrCr |
| | | | UrGrGrArGrG/AltR2/ |
| MG29-1 | 5543 | MG29-1- | /AltR1/rUrArArUrUrUr |
| sgRNA | | HPRT- | CrUrArCrUrGrUrUrGrUr |
| targeting | | sgRNA-F8 | ArGrArUrGrGrGrUrArAr |
| HPRT | | | ArArCrArArCrUrArGrUr |
| | | | GrUrGrCrCrA/AltR2/ |
| MG29-1 | 5544 | MG29-1- | /AltR1/rUrArArUrUrUr |
| sgRNA | | HPRT- | CrUrArCrUrGrUrUrGrUr |
| targeting | | sgRNA-G8 | ArGrArUrGrGrCrArCrAr |
| HPRT | | | CrUrArGrUrUrGrUrUrUr |
| | | | UrArCrCrCrU/AltR2/ |
| MG29-1 | 5545 | MG29-1- | /AltR1/rUrArArUrUrUr |
| sgRNA | | HPRT- | CrUrArCrUrGrUrUrGrUr |
| targeting | | sgRNA-H8 | ArGrArUrGrCrArCrArCr |
| HPRT | | | UrArGrUrUrGrUrUrUrUr |
| | | | ArCrCrCrUrA/AltR2/ |
| MG29-1 | 5546 | MG29-1- | /AltR1/rUrArArUrUrUr |
| sgRNA | | HPRT- | CrUrArCrUrGrUrUrGrUr |
| targeting | | sgRNA-A9 | ArGrArUrCrCrCrUrArAr |
| HPRT | | | ArGrUrUrCrCrUrCrUrUr |
| | | | UrGrUrArArG/AltR2/ |
| MG29-1 | 5547 | MG29-1- | /AltR1/rUrArArUrUrUr |
| sgRNA | | HPRT- | CrUrArCrUrGrUrUrGrUr |
| targeting | | sgRNA-B9 | ArGrArUrGrGrGrArUrUr |
| HPRT | | | GrUrArUrUrUrCrCrArAr |
| | | | GrGrUrUrUrC/AltR2/ |
| MG29-1 | 5548 | MG29-1- | /AltR1/rUrArArUrUrUr |
| sgRNA | | HPRT- | CrUrArCrUrGrUrUrGrUr |
| targeting | | sgRNA-C9 | ArGrArUrCrArArGrGrUr |
| HPRT | | | UrUrCrUrArGrArCrUrGr |
| | | | ArGrArGrCrC/AltR2/ |
| MG29-1 | 5549 | MG29-1- | /AltR1/rUrArArUrUrUr |
| sgRNA | | HPRT- | CrUrArCrUrGrUrUrGrUr |
| targeting | | sgRNA-D9 | ArGrArUrUrArGrArCrUr |
| HPRT | | | GrArGrArGrCrCrCrUrUr |
| | | | UrUrCrArUrC/AltR2/ |
| MG29-1 | 5550 | MG29-1- | /AltR1/rUrArArUrUrUr |
| sgRNA | | HPRT- | CrUrArCrUrGrUrUrGrUr |
| targeting | | sgRNA-E9 | ArGrArUrCrArUrCrUrUr |
| HPRT | | | UrGrCrUrCrArUrUrGrAr |
| | | | CrArCrUrCrU/AltR2/ |
| MG29-1 | 5551 | MG29-1- | /AltR1/rUrArArUrUrUr |
| sgRNA | | HPRT- | CrUrArCrUrGrUrUrGrUr |
| targeting | | sgRNA-F9 | ArGrArUrArUrCrUrUrUr |
| HPRT | | | GrCrUrCrArUrUrGrArCr |
| | | | ArCrUrCrUrG/AltR2/ |
| MG29-1 | 5552 | MG29-1- | /AltR1/rUrArArUrUrUr |
| sgRNA | | HPRT- | CrUrArCrUrGrUrUrGrUr |
| targeting | | sgRNA-G9 | ArGrArUrCrUrCrArUrUr |
| HPRT | | | GrArCrArCrUrCrUrGrUr |
| | | | ArCrCrCrArU/AltR2/ |
| MG29-1 | 5553 | MG29-1- | /AltR1/rUrArArUrUrUr |
| sgRNA | | HPRT- | CrUrArCrUrGrUrUrGrUr |
| targeting | | sgRNA-H9 | ArGrArUrArCrArCrArCr |
| HPRT | | | CrCrArArGrGrArArArGr |
| | | | ArCrUrArUrG/AltR2/ |
| MG29-1 | 5554 | MG29-1- | /AltR1/rUrArArUrUrUr |
| sgRNA | | HPRT- | CrUrArCrUrGrUrUrGrUr |
| targeting | | sgRNA-A10 | ArGrArUrArArCrArCrAr |
| HPRT | | | CrCrCrArArGrGrArArAr |
| | | | GrArCrUrArU/AltR2/ |
| MG29-1 | 5555 | MG29-1- | /AltR1/rUrArArUrUrUr |
| sgRNA | | HPRT- | CrUrArCrUrGrUrUrGrUr |
| targeting | | sgRNA-B10 | ArGrArUrGrCrUrCrUrCr |
| HPRT | | | CrArUrUrUrCrArUrArGr |
| | | | UrCrUrUrUrC/AltR2/ |
| MG29-1 | 5556 | MG29-1- | /AltR1/rUrArArUrUrUr |
| sgRNA | | HPRT- | CrUrArCrUrGrUrUrGrUr |
| targeting | | sgRNA-C10 | ArGrArUrArUrArGrUrCr |
| HPRT | | | UrUrUrCrCrUrUrGrGrGr |
| | | | UrGrUrGrUrU/AltR2/ |
| MG29-1 | 5557 | MG29-1- | /AltR1/rUrArArUrUrUr |
| sgRNA | | HPRT- | CrUrArCrUrGrUrUrGrUr |
| targeting | | sgRNA-D10 | ArGrArUrCrUrUrGrGrGr |
| HPRT | | | UrGrUrGrUrUrArArArAr |
| | | | GrUrGrArCrC/AltR2/ |
| MG29-1 | 5558 | MG29-1- | /AltR1/rUrArArUrUrUr |
| sgRNA | | HPRT- | CrUrArCrUrGrUrUrGrUr |
| targeting | | sgRNA-E10 | ArGrArUrArUrCrCrGrUr |
| HPRT | | | GrCrUrGrArGrUrGrUrAr |
| | | | CrCrArUrGrG/AltR2/ |
| MG29-1 | 5559 | MG29-1- | /AltR1/rUrArArUrUrUr |
| sgRNA | | HPRT- | CrUrArCrUrGrUrUrGrUr |
| targeting | | sgRNA-F10 | ArGrArUrArUrUrUrCrAr |
| HPRT | | | UrCrCrGrUrGrCrUrGrAr |
| | | | GrUrGrUrArC/AltR2/ |
| MG29-1 | 5560 | MG29-1- | /AltR1/rUrArArUrUrUr |
| sgRNA | | HPRT- | CrUrArCrUrGrUrUrGrUr |
| targeting | | sgRNA-G10 | ArGrArUrGrArArArCrGr |
| HPRT | | | UrCrArGrUrCrUrUrCrUr |
| | | | CrUrUrUrUrG/AltR2/ |
| MG29-1 | 5561 | MG29-1- | /AltR1/rUrArArUrUrUr |
| sgRNA | | HPRT- | CrUrArCrUrGrUrUrGrUr |
| targeting | | sgRNA-H10 | ArGrArUrUrArGrArGrAr |
| HPRT | | | GrGrCrArCrArUrUrUrGr |
| | | | CrCrArGrUrA/AltR2/ |
| DNA sequence | 5562 | MG29-1- | TCTCCCTGGCTTACCTTTAG |
| of HPRT | | HPRT- | GA |
| target site | | target | |
| | | site-A1 | |
| DNA sequence | 5563 | MG29-1- | GAACACAAGCCCACCATTAA |
| of HPRT | | HPRT- | AA |
| target site | | target | |
| | | site-B1 | |
| DNA sequence | 5564 | MG29-1- | AATGGTGGGCTTGTGTTCTA |
| of HPRT | | HPRT- | AA |
| target site | | target | |
| | | site-C1 | |
| DNA sequence | 5565 | MG29-1- | ATGGTGGGCTTGTGTTCTAA |
| of HPRT | | HPRT- | AG |
| target site | | target | |
| | | site-D1 | |
| DNA sequence | 5566 | MG29-1- | CCCTAACAAAGATGGGTTTG |
| of HPRT | | HPRT- | TT |
| target site | | target | |
| | | site-E1 | |
| DNA sequence | 5567 | MG29-1- | AAGGCACCCAAATTAATAAC |
| of HPRT | | HPRT- | GC |
| target site | | target | |
| | | site-F1 | |
| DNA sequence | 5568 | MG29-1- | TAGCGTTATTAATTTGGGTG |
| of HPRT | | HPRT- | CC |
| target site | | target | |
| | | site-G1 | |
| DNA sequence | 5569 | MG29-1- | TCGAGTGTAGTCTGTTAGCC |
| of HPRT | | HPRT- | AC |
| target site | | target | |
| | | site-H1 | |
| DNA sequence | 5570 | MG29-1- | TTAGGGAGTTATGATGTTGT |
| of HPRT | | HPRT- | CC |
| target site | | target | |
| | | site-A2 | |
| DNA sequence | 5571 | MG29-1- | TAGGTGCAGGATCAATGACA |
| of HPRT | | HPRT- | GC |
| target site | | target | |
| | | site-B2 | |
| DNA sequence | 5572 | MG29-1- | GTAGGTGCAGGATCAATGAC |
| of HPRT | | HPRT- | AG |
| target site | | target | |
| | | site-C2 | |
| DNA sequence | 5573 | MG29-1- | CTGTCATTGATCCTGCACCT |
| of HPRT | | HPRT- | AC |
| target site | | target | |
| | | site-D2 | |
| DNA sequence | 5574 | MG29-1- | CAGCACAGTAATTCTCACCA |
| of HPRT | | HPRT- | AA |
| target site | | target | |
| | | site-E2 | |
| DNA sequence | 5575 | MG29-1- | GGTGAGAATTACTGTGCTGA |
| of HPRT | | HPRT- | AA |
| target site | | target | |
| | | site-F2 | |
| DNA sequence | 5576 | MG29-1- | TCCTGAATAGCATGGCAGAG |
| of HPRT | | HPRT- | GA |
| target site | | target | |
| | | site-G2 | |
| DNA sequence | 5577 | MG29-1- | CAAGGGGGCCCAAAATCCTC |
| of HPRT | | HPRT- | TG |
| target site | | target | |
| | | site-H2 | |
| DNA sequence | 5578 | MG29-1- | GCAAGGGGGCCCAAAATCCT |
| of HPRT | | HPRT- | CT |
| target site | | target | |
| | | site-A3 | |
| DNA sequence | 5579 | MG29-1- | GGGCCCCCTTGCAAAATTAA |
| of HPRT | | HPRT- | GA |
| target site | | target | |
| | | site-B3 | |
| DNA sequence | 5580 | MG29-1- | GGCCCCCTTGCAAAATTAAG |
| of HPRT | | HPRT- | AA |
| target site | | target | |
| | | site-C3 | |
| DNA sequence | 5581 | MG29-1- | ACTACAGACACAAAGAAGAT |
| of HPRT | | HPRT- | GC |
| target site | | target | |
| | | site-D3 | |
| DNA sequence | 5582 | MG29-1- | TTATTAAGTCGGCCTCACCT |
| of HPRT | | HPRT- | CC |
| target site | | target | |
| | | site-E3 | |
| DNA sequence | 5583 | MG29-1- | TATTAAGTCGGCCTCACCTC |
| of HPRT | | HPRT- | CT |
| target site | | target | |
| | | site-F3 | |
| DNA sequence | 5584 | MG29-1- | ATTAAGTCGGCCTCACCTCC |
| of HPRT | | HPRT- | TC |
| target site | | target | |
| | | site-G3 | |
| DNA sequence | 5585 | MG29-1- | TTAAGTCGGCCTCACCTCCT |
| of HPRT | | HPRT- | CA |
| target site | | target | |
| | | site-H3 | |
| DNA sequence | 5586 | MG29-1- | TATGTAGGGGTCAGGTAATG |
| of HPRT | | HPRT- | TT |
| target site | | target | |
| | | site-A4 | |
| DNA sequence | 5587 | MG29-1- | ATGTAGGGGTCAGGTAATGT |
| of HPRT | | HPRT- | TC |
| target site | | target | |
| | | site-B4 | |
| DNA sequence | 5588 | MG29-1- | TGTAGGGGTCAGGTAATGTT |
| of HPRT | | HPRT- | CT |
| target site | | target | |
| | | site-C4 | |
| DNA sequence | 5589 | MG29-1- | GAAAAAATCACGGTATCTGT |
| of HPRT | | HPRT- | CG |
| target site | | target | |
| | | site-D4 | |
| DNA sequence | 5590 | MG29-1- | AATCAGAGTAAGCCTTCTAG |
| of HPRT | | HPRT- | TG |
| target site | | target | |
| | | site-E4 | |
| DNA sequence | 5591 | MG29-1- | GGACAGGTACTATGAGAGTA |
| of HPRT | | HPRT- | TA |
| target site | | target | |
| | | site-F4 | |
| DNA sequence | 5592 | MG29-1- | AAATGTCAACCTACTGTGGC |
| of HPRT | | HPRT- | AT |
| target site | | target | |
| | | site-G4 | |
| DNA sequence | 5593 | MG29-1- | AATGTCAACCTACTGTGGCA |
| of HPRT | | HPRT- | TA |
| target site | | target | |
| | | site-H4 | |
| DNA sequence | 5594 | MG29-1- | GTTGTTCTTTCCTGGTATAT |
| of HPRT | | HPRT- | GC |
| target site | | target | |
| | | site-A5 | |
| DNA sequence | 5595 | MG29-1- | CTGGTATATGCTGTGGAATT |
| of HPRT | | HPRT- | GA |
| target site | | target | |
| | | site-B5 | |
| DNA sequence | 5596 | MG29-1- | AGCATGTCCTACCTGTGGCA |
| of HPRT | | HPRT- | AC |
| target site | | target | |
| | | site-C5 | |
| DNA sequence | 5597 | MG29-1- | GTGTTGCCACAGGTAGGACA |
| of HPRT | | HPRT- | TG |
| target site | | target | |
| | | site-D5 | |
| DNA sequence | 5598 | MG29-1- | TGTTGCCACAGGTAGGACAT |
| of HPRT | | HPRT- | GC |
| target site | | target | |
| | | site-E5 | |
| DNA sequence | 5599 | MG29-1- | CATACTGTAAATGGGTAACC |
| of HPRT | | HPRT- | GT |
| target site | | target | |
| | | site-F5 | |
| DNA sequence | 5600 | MG29-1- | CCATACTGTAAATGGGTAAC |
| of HPRT | | HPRT- | CG |
| target site | | target | |
| | | site-G5 | |
| DNA sequence | 5601 | MG29-1- | ATAAAGCTCCATCTCTAAGG |
| of HPRT | | HPRT- | CA |
| target site | | target | |
| | | site-H5 | |
| DNA sequence | 5602 | MG29-1- | GTGATACCTTTTCTGGAGCA |
| of HPRT | | HPRT- | TT |
| target site | | target | |
| | | site-A6 | |
| DNA sequence | 5603 | MG29-1- | CTGGAGCATTCCTGAGTTCA |
| of HPRT | | HPRT- | GG |
| target site | | target | |
| | | site-B6 | |
| DNA sequence | 5604 | MG29-1- | TGGAGCATTCCTGAGTTCAG |
| of HPRT | | HPRT- | GT |
| target site | | target | |
| | | site-C6 | |
| DNA sequence | 5605 | MG29-1- | TGCTGTGATTGGCTTGTTAT |
| of HPRT | | HPRT- | GT |
| target site | | target | |
| | | site-D6 | |
| DNA sequence | 5606 | MG29-1- | GCTGTGATTGGCTTGTTATG |
| of HPRT | | HPRT- | TT |
| target site | | target | |
| | | site-E6 | |
| DNA sequence | 5607 | MG29-1- | CTGTGATTGGCTTGTTATGT |
| of HPRT | | HPRT- | TC |
| target site | | target | |
| | | site-F6 | |
| DNA sequence | 5608 | MG29-1- | TTGGAAGAGTCATGAGGGAC |
| of HPRT | | HPRT- | AT |
| target site | | target | |
| | | site-G6 | |
| DNA sequence | 5609 | MG29-1- | CAGATGTTAAAGGCAGTCTC |
| of HPRT | | HPRT- | AA |
| target site | | target | |
| | | site-H6 | |
| DNA sequence | 5610 | MG29-1- | CTTGAGACTGCCTTTAACAT |
| of HPRT | | HPRT- | CT |
| target site | | target | |
| | | site-A7 | |
| DNA sequence | 5611 | MG29-1- | TTGAGACTGCCTTTAACATC |
| of HPRT | | HPRT- | TG |
| target site | | target | |
| | | site-B7 | |
| DNA sequence | 5612 | MG29-1- | TAACCCAAATGCTGCCTGTT |
| of HPRT | | HPRT- | GA |
| target site | | target | |
| | | site-C7 | |
| DNA sequence | 5613 | MG29-1- | ATAACCCAAATGCTGCCTGT |
| of HPRT | | HPRT- | TG |
| target site | | target | |
| | | site-D7 | |
| DNA sequence | 5614 | MG29-1- | TCAACAGGCAGCATTTGGGT |
| of HPRT | | HPRT- | TA |
| target site | | target | |
| | | site-E7 | |
| DNA sequence | 5615 | MG29-1- | CAACAGGCAGCATTTGGGTT |
| of HPRT | | HPRT- | AT |
| target site | | target | |
| | | site-F7 | |
| DNA sequence | 5616 | MG29-1- | AACAGGCAGCATTTGGGTTA |
| of HPRT | | HPRT- | TA |
| target site | | target | |
| | | site-G7 | |
| DNA sequence | 5617 | MG29-1- | GAACAAAAGCTGGAGGTGGT |
| of HPRT | | HPRT- | AT |
| target site | | target | |
| | | site-H7 | |
| DNA sequence | 5618 | MG29-1- | GGTAGAGTTGACTTATACCA |
| of HPRT | | HPRT- | CC |
| target site | | target | |
| | | site-A8 | |
| DNA sequence | 5619 | MG29-1- | GTAGAGTTGACTTATACCAC |
| of HPRT | | HPRT- | CT |
| target site | | target | |
| | | site-B8 | |
| DNA sequence | 5620 | MG29-1- | GGAACAAAAGCTGGAGGTGG |
| of HPRT | | HPRT- | TA |
| target site | | target | |
| | | site-C8 | |
| DNA sequence | 5621 | MG29-1- | TGGAACAAAAGCTGGAGGTG |
| of HPRT | | HPRT- | GT |
| target site | | target | |
| | | site-D8 | |
| DNA sequence | 5622 | MG29-1- | TTTTTGGAACAAAAGCTGGA |
| of HPRT | | HPRT- | GG |
| target site | | target | |
| | | site-E8 | |
| DNA sequence | 5623 | MG29-1- | GGGTAAAACAACTAGTGTGC |
| of HPRT | | HPRT- | CA |
| target site | | target | |
| | | site-F8 | |
| DNA sequence | 5624 | MG29-1- | GGCACACTAGTTGTTTTACC |
| of HPRT | | HPRT- | CT |
| target site | | target | |
| | | site-G8 | |
| DNA sequence | 5625 | MG29-1- | GCACACTAGTTGTTTTACCC |
| of HPRT | | HPRT- | TA |
| target site | | target | |
| | | site-H8 | |
| DNA sequence | 5626 | MG29-1- | CCCTAAAGTTCCTCTTTGTA |
| of HPRT | | HPRT- | AG |
| target site | | target | |
| | | site-A9 | |
| DNA sequence | 5627 | MG29-1- | GGGATTGTATTTCCAAGGTT |
| of HPRT | | HPRT- | TC |
| target site | | target | |
| | | site-B9 | |
| DNA sequence | 5628 | MG29-1- | CAAGGTTTCTAGACTGAGAG |
| of HPRT | | HPRT- | CC |
| target site | | target | |
| | | site-C9 | |
| DNA sequence | 5629 | MG29-1- | TAGACTGAGAGCCCTTTTCA |
| of HPRT | | HPRT- | TC |
| target site | | target | |
| | | site-D9 | |
| DNA sequence | 5630 | MG29-1- | CATCTTTGCTCATTGACACT |
| of HPRT | | HPRT- | CT |
| target site | | target | |
| | | site-E9 | |
| DNA sequence | 5631 | MG29-1- | ATCTTTGCTCATTGACACTC |
| of HPRT | | HPRT- | TG |
| target site | | target | |
| | | site-F9 | |
| DNA sequence | 5632 | MG29-1- | CTCATTGACACTCTGTACCC |
| of HPRT | | HPRT- | AT |
| target site | | target | |
| | | site-G9 | |
| DNA sequence | 5633 | MG29-1- | ACACACCCAAGGAAAGACTA |
| of HPRT | | HPRT- | TG |
| target site | | target | |
| | | site-H9 | |
| DNA sequence | 5634 | MG29-1- | AACACACCCAAGGAAAGACT |
| of HPRT | | HPRT- | AT |
| target site | | target | |
| | | site-A10 | |
| DNA sequence | 5635 | MG29-1- | GCTCTCCATTTCATAGTCTT |
| of HPRT | | HPRT- | TC |
| target site | | target | |
| | | site-B10 | |
| DNA sequence | 5636 | MG29-1- | ATAGTCTTTCCTTGGGTGTG |
| of HPRT | | HPRT- | TT |
| target site | | target | |
| | | site-C10 | |
| DNA sequence | 5637 | MG29-1- | CTTGGGTGTGTTAAAAGTGA |
| of HPRT | | HPRT- | CC |
| target site | | target | |
| | | site-D10 | |
| DNA sequence | 5638 | MG29-1- | ATCCGTGCTGAGTGTACCAT |
| of HPRT | | HPRT- | GG |
| target site | | target | |
| | | site-E10 | |
| DNA sequence | 5639 | MG29-1- | ATTTCATCCGTGCTGAGTGT |
| of HPRT | | HPRT- | AC |
| target site | | target | |
| | | site-F10 | |
| DNA sequence | 5640 | MG29-1- | GAAACGTCAGTCTTCTCTTT |
| of HPRT | | HPRT- | TG |
| target site | | target | |
| | | site-G10 | |
| DNA sequence | 5641 | MG29-1- | TAGAGAGGCACATTTGCCAG |
| of HPRT | | HPRT- | TA |
| target site | | target | |
| | | site-H10 | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 22—Additional MG29-1 Guide Chemistry Optimization

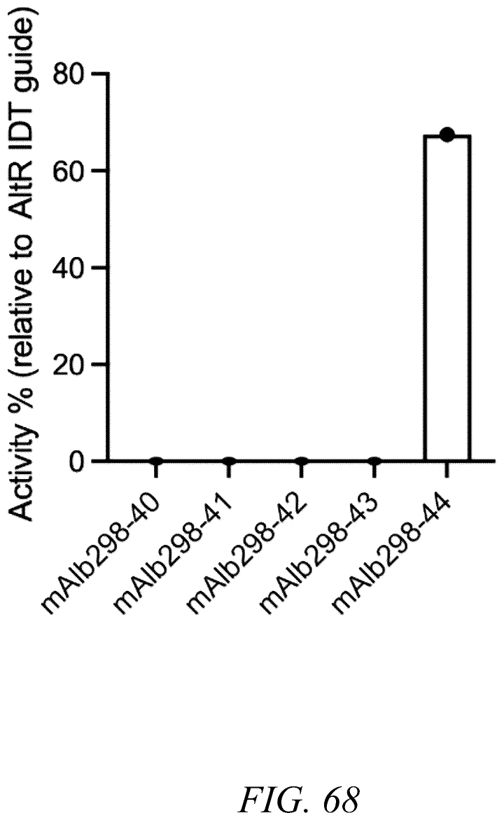

The editing activity of 5 guides with the same base sequence but different chemical modifications was evaluated in Hepa1-6 cells by co-transfection of mRNA encoding MG29-1 and the guide; the results are shown in Table 15 and FIG. 68. A guide with the same base sequence and a commercially available chemical modification called A1tR1/A1tR2 was used as a control. The spacer sequence in these guides targets a 22 nucleotide region in albumin intron1 of the mouse genome. Guide mA1b298-44 exhibited 67.5% of the editing activity of the control AltR1/AltR2 guide while the other 4 guides did not result in measurable editing. When co-transfection of mRNA and guide with a lipid transfection reagent such as Messenger MAX is used, the mixture of the two RNA forms a complex with the positively charged lipid and the complex enters the cells via endocytosis and eventually reaches the cytoplasm, where the mRNA is translated into protein. In the case of an RNA guided nuclease such as MG29-1, the resulting MG29-1 protein presumably forms a complex with the guide RNA in the cytoplasm before entering the nucleus in a process mediated by the nuclear localization signals that were engineered into the MG29-1 protein. Because translation of the mRNA into sufficient amounts of MG29-1 protein followed by the binding of the MG29-1 protein to the guide RNA takes a finite amount of time, the guide RNA may require increased stability in the cytoplasm for longer than is the case when pre-formed RNP is delivered by nucleofection. Thus, lipid-based mRNA/sgRNA co-transfection may require a more stable guide than is the case for RNP nucleofection, which may result in some guide chemistries being active as RNP but inactive when co-transfected with mRNA using cationic lipid reagents.

TABLE 15
Activity of chemically modified MG29-1
guides in Hepa1-6 cells transfected with
MG29-1 mRNA and the guide RNA

| | | | Editing |
|---|---|---|---|
| | | sgRNA sequence | activity |
| | SEQ | and | (% of |
| sgRNA | ID | chemical | AltR1/AltR2 |
| name | No. | modifications | control) |
| mAlb298-40 | 5745 | mC*mU*mU*U*UAAU | 0 |
| | | UmUmCmUmACU*G*U | |
| | | *U*GUAGAUi2FCi2 | |
| | | FUI2FGi2FUi2FAi | |
| | | 2FAi2FCi2FGi2FA | |
| | | i2FUi2FCi2FGi2F | |
| | | Gi2FGi2FAi2FAi2 | |
| | | FC*i2FU*i2FGi2F | |
| | | G*i2FC*mA | |
| mAlb298-41 | 5746 | mC*mU*mU*U*UAAU | 0 |
| | | UmUmCmUmACU*G*U | |
| | | *U*dGdTdAdGdAdT | |
| | | i2FCi2FUi2FGi2F | |
| | | Ui2FAi2FAi2FCi2 | |
| | | FGi2FAi2FUi2FCi | |
| | | 2FGi2FGi2FGi2FA | |
| | | i2FAi2FC*i2FU*i | |
| | | 2FGi2FG*i2FC*MA | |
| mAlb298-42 | 5747 | mC*mU*mU*U*UAAU | 0 |
| | | UmUmCmUmACU*G*U | |
| | | *U*i2FGi2FUi2FA | |
| | | i2FGi2FAi2FUi2F | |
| | | Ci2FUi2FGi2FUi2 | |
| | | FAi2FAi2FCi2FGi | |
| | | 2FAi2FUi2FCi2FG | |
| | | i2FGi2FGi2FAi2F | |
| | | Ai2FC*i2FU*i2FG | |
| | | i2FG*12FC*mA | |
| mAlb298-43 | 5748 | mC*mU*mU*U*UAAU | 0 |
| | | UmUmCmUmAmCU*G* | |
| | | U*U*GUAGAUi2FCi | |
| | | 2FUI2FGi2FUi2FA | |
| | | i2FAi2FCi2FGi2F | |
| | | Ai2FUi2FCi2FGi2 | |
| | | FGi2FGi2FAi2FAi | |
| | | 2FC*12FU*i2FGi2 | |
| | | FG*i2FC*MA | |

Nomenclature of chemical modifications: a “/” is used to separate bases with 2′-flourine modifications, m; 2′-O-methyl base (for example a A base with 2′-O-methyl modification is written as mA), i2F; internal 2′-flourine base (for example an internal C with 2′-flourine modification is written as /12FC/), 52F; 2′-flourine base at the 5′ end of the sequence (for example a 5′ C with 2′-flourine modification is written as /52FC/), 32F; 2′-flourine base at the 3′ end of the sequence (for example a 3′ A base with 2′- flourine modification is written as /32FA/), r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to DDT technologies' proprietary 5′ and 3′ AltR modifications

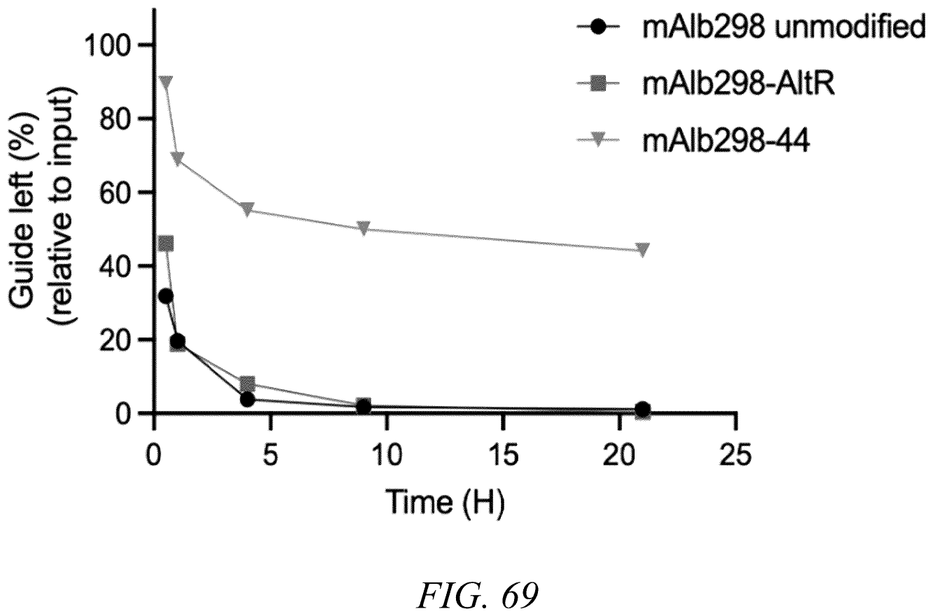

In order to test the stability of these chemically modified guides compared to the guide with no chemical modification (native RNA), a stability assay using crude cell extracts was used. Crude cell extracts from mammalian cells were selected because they contain the mixture of nucleases that a guide RNA will be exposed to when delivered to mammalian cells in vitro or in vivo. Hepa1-6 cells were collected by adding 3 ml of cold PBS per 15 cm dish of confluent cells and releasing the cells from the surface of the dish using a cell scraper. The cells were pelleted at 200 g for 10 min and frozen at −80° C. for future use. For the stability assays, cells were resuspended in 4 volumes of cold PBS (i.e. for a 100 mg pellet cells were resuspended in 400 ul of cold PBS). Triton X-100 was added to a ending concentration of 0.2% (v/v), cells were vortexed for 10 seconds, put on ice for 10 minutes and vortexed again for 10 seconds. Triton X-100 is a mild non-ionic detergent that disrupts cell membranes but does not inactivate or denature proteins at the concentration used. Stability reactions were set up on ice and comprised 20 ul of cell crude extract with 2 pmoles of each guide (1 ul of a 2 uM stock). Six reactions were set up per guide comprising: input, 0.5 hour, 1 hour, 4 hours, 9 hours and 21 hours (the time in hours referring to the length of time each sample was incubated). Samples were incubated at 37° C. from 0.5 hours up to 21 hours while the input control was left on ice for 5 minutes. After each incubation period, the reaction was stopped by adding 300 ul of a mixture of phenol and guanidine thiocyanate (Tri reagent, Zymo Research) which immediately denatures all proteins and efficiently inhibits ribonucleases and facilitates the subsequent recovery of RNA. After adding Tri Reagent the samples were vortexed for 15 seconds and stored at −20° C. RNA was extracted from the samples using Direct-zol RNA miniprep kit (Zymo Research) and eluted in 100 ul of nuclease-free water. Detection of the modified guide was performed using Taqman RT-qPCR using the Taqman miRNA Assay technology (Thermo Fisher) and primers and probes designed to specifically detect the sequence in the mA1b298 sgRNA, which is the same for all of the guides. Data was plotted as a function of percentage of sgRNA remaining in relation to the input sample (Table 16 and FIG. 69).

TABLE 16
Stability of Chemically Modified MG29-1 Guides

| | Percentage of guide left | | |
|---|---|---|---|
| Time | mAlb298 | mAlb298- | mAlb298- |
| (H) | unmodified | AltR | 44 |
| 0.5 | 31.8640157 | 46.1691 | 89.5025 |
| 1 | 19.6826994 | 18.8809 | 68.7771 |
| 4 | 3.80753207 | 7.99366 | 55.0953 |
| 9 | 1.67464604 | 2.14928 | 50 |
| 21 | 1.17190546 | 0.4044 | 44.1351 |

Guide mA1b289-44 exhibited significantly improved stability in the cell lysate compared to both un-modified guide and the guide with AltR1/AltR2 modifications. Thus, the chemical modifications present in the mA1b289-44 guide may be useful for optimizing editing in vivo. The chemical modifications present on the mA1b289-44 guide are detailed in Table 15. The mA1b289-44 guide chemistry differs from another highly stable guide chemistry called the mA1b289-37 by the presence of 3 additional phosphorothioate linkages

### Example 23—Improving the Stability of the Guide RNA for MG29-1 by Addition of a Stem-Loop at the 5′ End

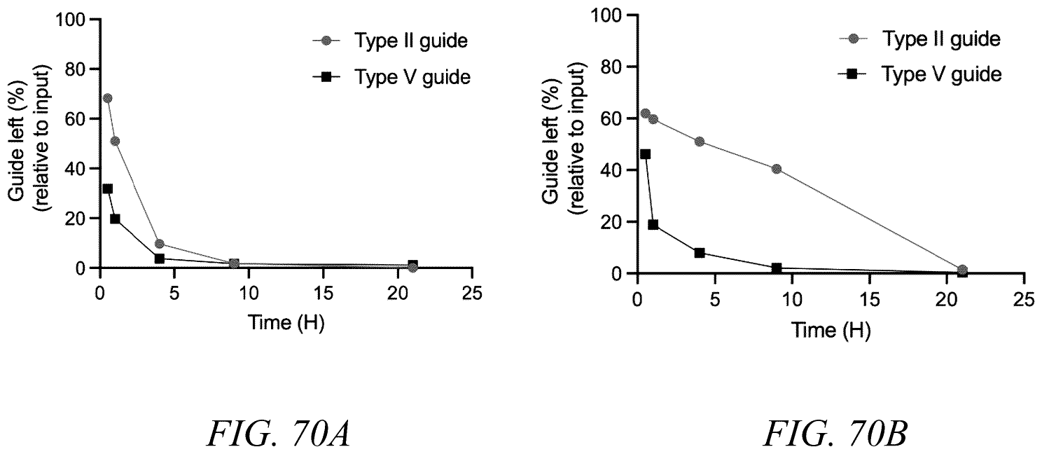

A comparison of the stability in cell lysates in vitro of the guide RNA for MG29-1 to that of the guide RNA for a type II CRISPR nuclease called MG3-6/3-4 shows that the MG29-1 guide is inherently less stable (Table 17 and FIGS. 70A-B).

TABLE 17
Stability of Type II Versus Type V Guides

| | Percentage of guide left | | | |
|---|---|---|---|---|
| | Unmodified | | Only 5′ and 3′ End Modifications | |
| Time (H) | Type II | Type V | Type II | Type V |
| 0.5 | 68.30201 | 31.86402 | 61.98539 | 46.16912 |
| 1 | 51.05061 | 19.6827 | 59.66679 | 18.88091 |
| 4 | 9.672281 | 3.807532 | 51.05061 | 7.99366 |
| 9 | 1.757904 | 1.674646 | 40.47211 | 2.149284 |
| 21 | 0.034051 | 1.171905 | 1.447794 | 0.4044 |

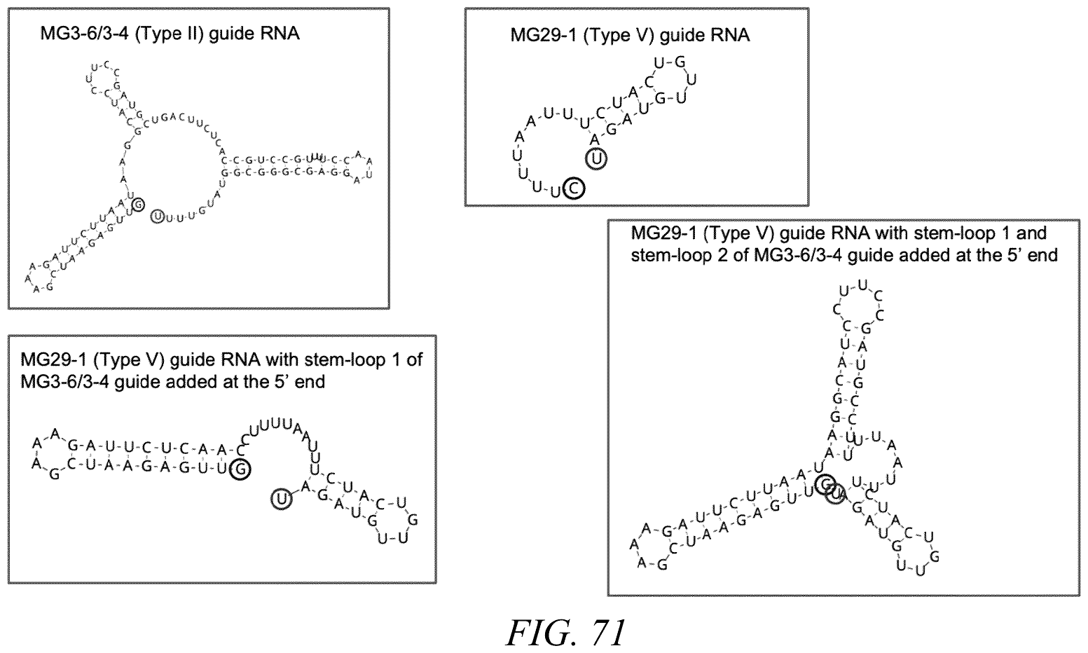

As shown in FIG. 70A, when comparing guide RNA without chemical modifications, the Type V guide (MG29-1 guide) was degraded more rapidly than the Type II guide (MG3-6/3-4 guide). As shown in FIG. 70B, when comparing guide RNA with chemical modifications of both ends of the RNA, the difference in stability between the Type V guide (MG29-1 guide) and the Type II guide (MG3-6/3-4 guide) was even more pronounced. The end-modified type V guide was almost completely degraded in 10 h while 40% of the Type II guide remained at the same time points. The secondary structures of the backbone (CRISPR repeat and tracr) of the MG29-1 (Type V) guide and the backbone of the MG3-6/3-4 (Type II) guide were predicted using the folding algorithm in Geneious Prime and are shown in FIG. 71. The backbone of the MG29-1 guide is 24 nucleotides long while that of MG3-6/3-4 is 88 nucleotides long. The backbone (CRISPR repeat) of the MG29-1 guide is predicted to form a single stem loop with a stem comprised of 5 nucleotides and a free energy of −1.22 kcal/mol while the backbone (CRISPR repeat and tracr) of MG3-6/3-4 is predicted to form 3 stem loops with a free energy of −14.8 kcal/mol. The three stem-loops of the MG3-6/3-4 guide RNA are comprised of stem 1 (at the 5′ end of the TRACR) that has a 10 nucleotide stem, stem 2 (in the middle) that has a 5 nucleotide stem, and stem 3 (at the 3′ end of the backbone) that has a 11 nucleotide stem. The larger size and more extensive stem structures in the MG3-6/3-4 guide RNA may contribute to the greater stability of this guide compared to the MG29-1 guide. The addition of a stem-loop forming RNA sequence at the 5′ end of the MG29-1 guide may improve its stability and thereby potentially improve the efficiency of editing in vivo. In one embodiment, stem 1 of the MG3-6/3-4 guide comprises the sequence (GUUGAGAAUCGAAAGAUUCUUAAU), wherein the underlined bases are predicted to form non-canonical G-U base pairs. To improve the stability of the stem, the underlined bases were changed from U to C to convert these to G-C base pairs in the predicted stem (GUUGAGAAUCGAAAGAUUCUCAAC). In one embodiment, this stem-loop forming sequence is added at the 5′ end of the MG29-1 guide RNA with chemistry #37. In addition, the chemical modifications on the 5′-most 4 nucleotides of the #37 chemistry (comprised of 2′ O-methyl and phosphorothioate linkages) were replicated at the new 5′ end of the guide in order to protect the 5′ end of the guide from nuclease attack. This gave rise to the guide RNA sequence called mA1b29-8-50 (Table 18). In an alternative guide design, the chemically modified bases at the original 5′ end of the mA1b298-37 were moved to the new 5′ end of the guide after the addition of the stem-loop sequence as in mA1b29-8-49. In another design for a potentially more stable MG29-1 guide, the RNA sequence from the MG3-6/3-4 backbone that encompasses stem-loop 1 and stem-loop 2 was added to the 5′ end of the MG29-1 guide to create mA1b29-8-48 and mA1b29-8-47, which differ in the chemically modified bases included. In another version, guide mA1b29-8-48 is further chemically modified by inclusion of phosphorothioate and 2′ O-methyl bases in the loop 1 (mALb29-8-47) or loop 1 and loop 2 ((mALb29-8-46). The activity of these guides can be tested in mammalian cells by transfection of mRNA and guide RNA mixtures using MessengerMax lipid reagent or other methodologies. The stability of these guides can be tested in the same mammalian cell lysate assay system as described above. Guides that retain editing activity and exhibit improvements in stability are candidates for testing in vivo in mice.

TABLE 18
Activity of chemically modified MG29-1
guides in Hepa1-6 cells transfected with
MG29-1 mRNA and the guide RNA

| | SEQ | sgRNA sequence and |
|---|---|---|
| | ID | chemical |
| sgRNA name | NO. | modifications |
| mAlb298-37 | 5750 | mC*mU*mU*U*rUrArArUrUmUmC |
| | | mUmArCrU*rG*rU*rU*rGrUrA |
| | | rGrArUrCrUrGrUrArArCi2FGi |
| | | 2FAi2FUi2FCi2FGi2FGi2FGi |
| | | 2FAi2FAi2FC*i2FU*i2FGi2FG |
| | | *i2FC*MA |
| mAlb29-8-50 | 5751 | mG*mU*mU*GrArGrArArUrC*mG |
| | | *mA*mA*mArGrArUrUrCrUrCr |
| | | ArArC*mC*mU*mU*U*UrArArU |
| | | rUmUmCmUmArCrU*G*U*U*GrU |
| | | rArGrArUrCrUrGrUrArArCfGf |
| | | AfUfCfGfGfGfAfAfC*fU*fGf |
| | | G*fC*mA |
| mAlb29-8-49 | 5752 | mG*mU*mU*GrArGrArArUrCrGr |
| | | ArArArGrArUrUrCrUrCrArAr |
| | | C*mC*mU*mU*U*UrArArUrUmUm |
| | | CmUmArCrU*G*U*U*GrUrArGr |
| | | ArUrCrUrGrUrArArCfGfAfUfC |
| | | fGfGfGfAfAfC*fU*fGfG*fC* |
| | | mA |
| mAlb29-8-48 | 5753 | mG*mU*mU*GAGAAUCGAAAGAUUC |
| | | UUAAUAAGGCAUCCUUCCGAUGCm |
| | | C*mU*mU*U*rUrArArUrUmUmCm |
| | | UmArCrU*rG*rU*rU*rGrUrAr |
| | | GrArUrCrUrGrUrArArCi2FGi |
| | | 2FAi2FUi2FCi2FGi2FGi2FGi2 |
| | | FAi2FAi2FC*12FU*i2FGi2FG |
| | | *i2FC*mA |
| mAlb29-8-47 | 5754 | mG*mU*mU*GAGAAUCGAAAGAUUC |
| | | UUAAUAAGGCAUCCUUCCGAUGCC |
| | | UUUrUrArArUrUmUmCmUmArCrU |
| | | *rG*rU*rU*rGrUrArGrArUrC |
| | | rUrGrUrArArCi2FGi2FAi2FU |
| | | I2FCi2FGi2FGi2FGi2FAi2FAi |
| | | 2FC*12FU*i2FGi2FG*i2FC*m |
| | | A |
| mAlb29-8-46 | 5755 | mG*mU*mU*GAGAAUCmG*mA*mA* |
| | | MAGAUUCUUAAUAAGGCAUCmC*m |
| | | U*mU*mC*mCGAUGCmC*mU*mU*U |
| | | *rUrArArUrUmUmCmUmArCrU* |
| | | rG*rU*rU*rGrUrArGrArUrCr |
| | | UrGrUrArArCi2FGi2FAi2FUi2 |
| | | FCi2FGi2FGi2FGi2FAi2FAi2 |
| | | FC*i2FU*i2FGi2FG*i2FC*MA |

Nomenclature of chemical modifications: a “/” is used to separate bases with 2′-flourine modifications, m; 2′-O-methyl base (for example a A base with 2′-O-methyl modification is written as mA), i2F; internal 2′-flourine base (for example an internal C with 2′-flourine modification is written as /12FC/), 52F; 2′-flourine base at the 5′ end of the sequence (for example a 5′ C with 2′-flourine modification is written as /52FC/), 32F; 2′-flourine base at the 3′ end of the sequence (for example a 3′ A base with 2′-flourine modification is written as /32FA/), r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 24—In Vivo Genome Editing with the MG29-1 Nuclease

To evaluate the ability of the MG29-1 Type V nuclease to edit the genome in vivo in a living animal, we used a lipid nanoparticle to deliver a mRNA encoding the MG29-1 nuclease and one of four guide RNA. The four guide RNA tested are mA1b298-37, mA1b2912-37, mA1b2918-37, and mA1b298-34, the sequences of which are shown in Table 19. Guides mA1b298-37 and mA1b298-34 have the same nucleotide sequence but different chemical modifications while guides mA1b298-37, mA1b2912-37, and mA1b2918-37 have different spacer sequences but the same chemical modifications.

TABLE 19
Sequences and chemical modifications of
guide RNA tested in vivo in mice

| | SEQ | |
|---|---|---|
| | ID | |
| Guide name | NO. | Sequence |
| mAlb298-37 | 5756 | mC*mU*mU*U*UAAUUmUmC |
| | | mUmACU*G*U*U*GUAGAUC |
| | | UGUAACfGfAfUfCfGfGfG |
| | | fAfAfC*fU*fGfG*fC*mA |
| mAlb2912-37 | 5757 | mC*mU*mU*U*UAAUUmUmC |
| | | mUmACU*G*U*U*GUAGAUA |
| | | GUGUAGfCfAfGfAfGfAfG |
| | | fGfAfA*fC*fCfA*fU*mU |
| mAlb2918-37 | 5758 | mC*mU*mU*U*UAAUUmUmC |
| | | mUmACU*G*U*U*GUAGAUA |
| | | AGAUUGfAfUfGfAfAfGfA |
| | | fCfAfA*fC*fUfA*fA*mC |
| mAlb298-34 | 5759 | mC*rU*rUrArArUrUmUmC |
| | | mUmArCrUrGrUrUrGmUmA |
| | | mGmArUrCrUrGrUrArArC |
| | | rGrArUrCrGrGrGrA*rAf |
| | | C*fUfG*fGfC*mA |

Nomenclature of chemical modifications: a “/” is used to separate bases with 2'-flourine modifications, m; 2'-O-methyl base (for example a A base with 2'-O-methyl modification is written as mA), i2F; internal 2'-flourine base (for example an internal C with 2'-flourine modification is written as /i2FC/), 52F; 2'-flourine base at the 5' end of the sequence (for example a 5' C with 2'-flourine modification is written as /52FC/), 32F; 2'-flourine base at the 3' end of the sequence (for example a 3' A base with 2'-flourine modification is written as /32FA/), r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5' and 3' AltR modifications

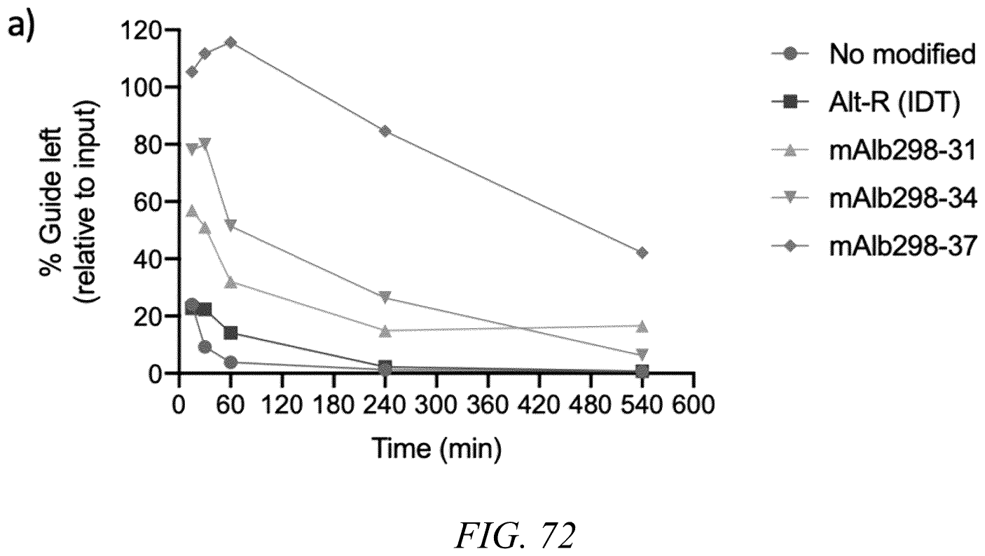

In the in vitro stability assay in Hepa1-6 cell lysates, the mA1b298-37 guide was more stable than the mA1b298-34 guide (FIG. 72), demonstrating that the chemical modifications on the mA1b298-37 guide were more effective at protecting the guide RNA against degradation.

The mRNA encoding MG29-1 was generated by in vitro transcription of a linearized plasmid template using T7 RNA polymerase and standard conditions using nucleotides and enzymes purchased from New England Biolabs or Trilink Biotechnologies. The sequence of the MG29-1 coding sequence is shown in SEQ ID No. 5680. The protein coding sequence of the MG29-1 cassette comprises the following elements from 5′ to 3′: the nuclear localization signal from SV40, a five amino acid linker(GGGS), the protein coding sequence of the MG29-1 nuclease from which the initiating methionine codon was removed, a 3 amino acid linker (SGG) and the nuclear localization signal from nucleoplasmin. The DNA sequence of this cassette was codon optimized for human using a commercially available algorithm. An approximately 100 nucleotide polyA tail was encoded in the plasmid used for in vitro transcription, and the mRNA was co-transcriptionally capped using the CleanCAP (™) reagent purchased from Trilink Biotechnologies. Uridine in the mRNA was replaced with N1-methyl pseudouridine.

The lipid nanoparticle (LNP) formulation used to deliver the MG29-1 mRNA and the guide RNA is based on LNP formulations described in the literature including Kauffman et al. (see e.g. Nano Lett. 2015, 15, 11, 7300-7306, which is incorporated by reference herein). The four lipid components were dissolved in ethanol and mixed in an appropriate molar ratio to make the lipid working mix. The mRNA and the guide RNA were either mixed before formulation at a 1:1 mass ratio or formulated in separate LNP that were later co-injected into mice at a 1:1 mass ratio of the two RNA's. In either case, the RNA was diluted in 100 mM Sodium Acetate (pH4.0) to make the RNA working stock. The lipid working stock and the RNA working stock were mixed in a microfluidics device (Ignite NanoAssembler, Precision Nanosystems) at a flow rate ratio of 1:3, respectively, and a flow rate of 12 mls/min. The LNP were dialyzed against phosphate buffered saline (PBS) for 2 to 16 hours and then concentrated using Amicon spin concentrators (Millipore) until the ending volume was achieved. The concentration of RNA in the LNP formulation was measured using the Ribogreen reagent (Thermo Fisher). The diameter and polydispersity (PDI) of the LNP were determined by dynamic light scattering. Example LNP had diameters ranging from 65 nm to 120 nm and PDI of 0.05 to 0.20. LNP were injected intravenously into 8 to 12 week old C57B16 wild type mice via the tail vein (0.1 ml per mouse) at a total RNA dose of 1 mg RNA per kg body weight. The mice were sacrificed three days post dosing, and the liver was collected and homogenized using a bead beater (Omni International) in a digestion buffer supplied in the PureLink Genomic DNA Isolation Kit (Thermo Fisher Scientific). Genomic DNA was purified from the resulting homogenate using the PureLink Genomic DNA Isolation Kit (Thermo Fisher Scientific) and quantified by measuring the absorbance at 260 nm. Genomic DNA purified from mice injected with buffer alone was used as a control. The liver genomic DNA was then PCR amplified using primers flanking the region targeted by the guides. The PCR primers used are shown in Table 20. PCR was performed using Pfusion flash high fidelity PCR master mix (Thermo Fisher Scientific) on 50 ng of genomic DNA and an annealing temperature of 64° C.

TABLE 20
Sequences of PCR primers and Sequencing
primers used to analyze in vivo
genome editing in mice

| Primer name | SEQ ID NO. | Sequence |
|---|---|---|
| mAlb90F | 5760 | CTCCTCTTCGTCTCCGGC |
| mAlb1073 | 5761 | CTGCCACATTGCTCAGCAC |
| mALb460F | 5762 | GCCTGCTCGACCATGCTAT |
| | | A |

The resulting PCR product was a single band by agarose gel electrophoresis and was purified using the DNA Clean & Concentrator-5 kit (Zymo Research), then subjected to Sanger sequencing with the primer mA1b460F that is located between 100 and 300 bases from the target sites of the different guides. The Sanger sequencing chromatograms were analyzed for insertions and deletions (INDELS) at the predicted target site for each guide by Tracking of Indels by DEcomposition (TIDE) as described by Brinkman et al (Nucleic Acids Res. 2014 Dec. 16; 42 (22): e168)_The presence of INDELS at the target site is the consequence of the generation of double strand breaks in the DNA, which are then repaired by the error prone cellular repair machinery which introduces insertions and deletions.

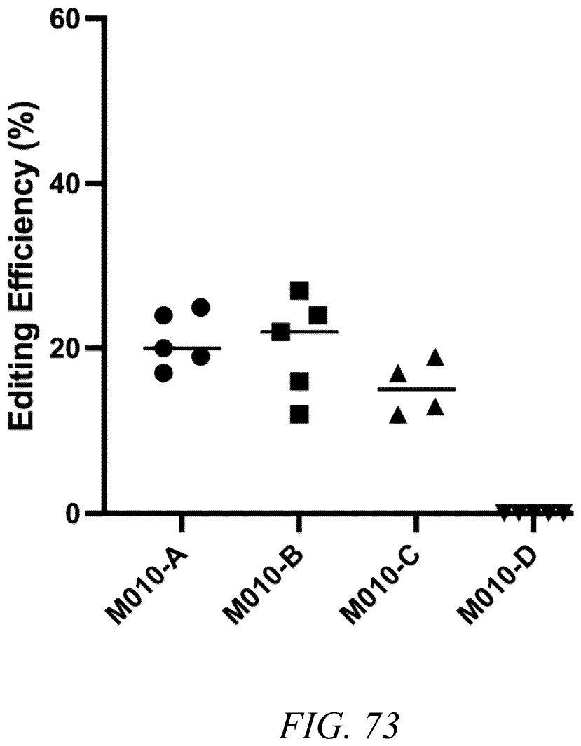

The results of the TIDE analysis are shown in FIG. 73 and Table 21. Group A mice received LNP encapsulating guide RNA mA1b298-37. Group B mice received LNP encapsulating guide RNA mA1b2912-37. Group C mice received LNP encapsulating guide RNA mA1b2918-37. Group D mice received LNP encapsulating guide RNA mA1b298-34. All mice also received LNP encapsulating the MG29-1 mRNA. The average INDEL frequency in group A that received guide mA1b298-37 was 21%. The average INDEL frequency in group B that received guide mA1b2912-37 was 20%. The average INDEL frequency in group C that received guide mA1b2918-37 was 15%. The average INDEL frequency in group D that received guide mA1b298-34 was 0%. This data demonstrates that the MG29-1 nuclease together with a guide RNA comprised of chemical modified bases (chemistry #37) was active in vivo in the liver of mice. Guide mA1b298-34 that has the same nucleotide sequence as guide mA1b298-37, but with different chemical modifications, was not active. Guide mA1b298-34 exhibited less stability in cell lysate than guide mA1b298-37, which correlates to in vivo activity.

TABLE 21
Gene editing at the on target site in the liver of mice at 3 days after IV
injection of nuclease mRNA and guide RNA packaged in LNP

| | | Editing | |
|---|---|---|---|
| Group | Animal | Efficiency (%) | R-Squared |
| A | 1281 | 20 | 0.98 |
| | 1282 | 19 | 0.98 |
| | 1283 | 25 | 0.98 |
| | 1284 | 24 | 0.98 |
| | 1285 | 17 | 0.99 |
| B | 1286 | 24 | 0.97 |
| | 1287 | 12 | 0.98 |
| | 1288 | 16 | 0.98 |
| | 1289 | 22 | 0.97 |
| | 1290 | 27 | 0.97 |
| C | 1291 | 12 | 0.99 |
| | 1292 | 19 | 0.99 |
| | 1293 | Not analyzed | |
| | 1294 | 13 | 0.99 |
| | 1295 | 17 | 0.99 |
| D | 1296 | 0 | 1 |
| | 1297 | 0 | 1 |
| | 1298 | 0 | 1 |
| | 1299 | 0 | 1 |
| | 1300 | 0 | 1 |

### Example 25—MG29-1 Guide Screen for Mouse HAO-1 Gene Using mRNA Transfection

From a guide screen of exons 1 to 4 of the mouse HAO-1 gene that was performed using MG29-1 protein complexed to the guide RNA that was nucleofected into Hepa1-6 cells, 5 highly active guides were selected for further evaluation by transfection of mRNA encoding MG29-1 mixed with the guide RNA. 300 ng mRNA and 120 ng single guide RNA were transfected into Hepa1-6 cells as follows. One day before to transfection, Hepa1-6 cells that have been cultured for less than 10 days in DMEM, 10% FBS, 1×NEAA media, without Pen/Strep, were seeded into a TC-treated 24 well plate. Cells were counted, and the equivalent volume to 60,000 viable cells were added to each well. Additional pre-equilibrated media was added to each well to bring the total volume to 500 μL. On the day of transfection, 25 μL of OptiMEM media and 1.25 ul of Lipofectamine Messenger Max Solution (Thermo Fisher) were mixed in a mastermix solution, vortexed, and allowed to sit for at least 5 minutes at room temperature. In separate tubes, 300 ng of the MG29-1 mRNA and 120 ng of the sgRNA were mixed together with 25 μL of OptiMEM media, and vortexed briefly. The appropriate volume of MessengerMax solution was added to each RNA solution, mixed by flicking the tube and briefly spun down at a low speed. The complete editing reagent solutions were allowed to incubate for 10 minutes at room temperature, then added directly to the Hepa1-6 cells. Two days post transfection, the media was aspirated off of each well of Hepa1-6 cells and genomic DNA was purified by automated magnetic bead purification, via the KingFisher Flex with the MagMAX™ DNA Multi-Sample Ultra 2.0 Kit. The activity of the guides is summarized in Table 22, while the primers used are summarized in Table 23.

TABLE 22
Average Activity of MG29-1 guides at
mouse HAO1 delivered by mRNA
Transfection

| | | | | Editing |
|---|---|---|---|---|
| | | SEQ | | Activity |
| Guide | | ID | Spacer | (Average |
| Name | PAM | NO. | Sequence | % INDELs) |
| mH29-1 | TTTG | 5763 | CCCCAGACCTGTA | 26.0 |
| | | | ATAGTCATA | |
| mH29-15 | TTTG | 5764 | TGACTGTGGACAC | 32.7 |
| | | | CCCTTACCT | |
| mH29-16 | TTTC | 5765 | ATTACAGCCTGTC | 15.0 |
| | | | AGACCATGG | |
| mH29-26 | \|TTTC | 5766 | TCCATTTCATTAC | 10.0 |
| | | | AGCCTGTCA | |
| mH29-29 | TTTC | 5767 | CCTTAGGAGAAAA | 36.7 |
| | | | TGCCAAATC | |

TABLE 23
Primers designed for the mouse HAO1 gene,
used for PCR at each of the first
four exons, and for sanger sequencing

| | | | SEQ | Primer |
|---|---|---|---|---|
| Target | | Primer | ID | Se- |
| Exon | Use | Name | NO: | quence |
| Mouse | Fwd PCR | PCR_mHE1_ | 5768 | GTGACC |
| HA01 | | F_+233 | | AACCCT |
| Exon 1 | | | | ACCCGT |
| | | | | TT |
| | Rev PCR | PCR_mHE1_ | 5769 | GCAAGC |
| | | R_−553 | | ACCTAC |
| | | | | TGTCTC |
| | | | | GT |
| | Sequencing | Seq_mHE1_ | 5770 | GTCTAG |
| | | F_+139 | | GCATAC |
| | | | | AATGTT |
| | | | | TGCTCA |
| Mouse | Fwd PCR | HA01_E2_ | 5771 | CAACGA |
| HA01 | | F5721 | | AGGTTC |
| Exon 2 | | | | CCTCCA |
| | | | | GG |
| | Rev PCR | HA01_E2_ | 5772 | GGAAGG |
| | | R6271 | | GTGTTC |
| | | | | GAGAAG |
| | | | | GA |
| | Sequencing | 5938F_Seq_ | 5773 | CTATGC |
| | | HAO1_E2 | | AAGGAA |
| | | | | AAGATT |
| | | | | TGGCC |
| Mouse | Fwd PCR | HA01_E3_ | 5774 | TGCCCT |
| HA01 | | F23198 | | AGACAA |
| Exon 3 | | | | GCTGAC |
| | | | | AC |
| | Rev PCR | HA01_E3_ | 5775 | CAGATT |
| | | R23879 | | CTGGAA |
| | | | | GTGGCC |
| | | | | CA |
| | Sequencing | HA01_E3_ | 5774 | Same as |
| | | F23198 | | Fwd |
| | | | | PCR |
| | | | | Primer |
| Mouse | Fwd PCR | PCR_mHE4_ | 5776 | GGCTGG |
| HA01 | | F_+300 | | CTGAAA |
| Exon 4 | | | | ATAGCA |
| | | | | TCC |
| | Rev PCR | HA01_E4_ | 5777 | AGGTTT |
| | | R31650 | | GGTTCC |
| | | | | CCTCAC |
| | | | | CT |
| | Sequencing | PCR_mHE4_ | 5778 | TCTGCC |
| | | R_−149 | | ATGAAG |
| | | | | GCATAT |
| | | | | GGAC |

### Example 52—Efficiency of mRNA Electroporation in T Cells

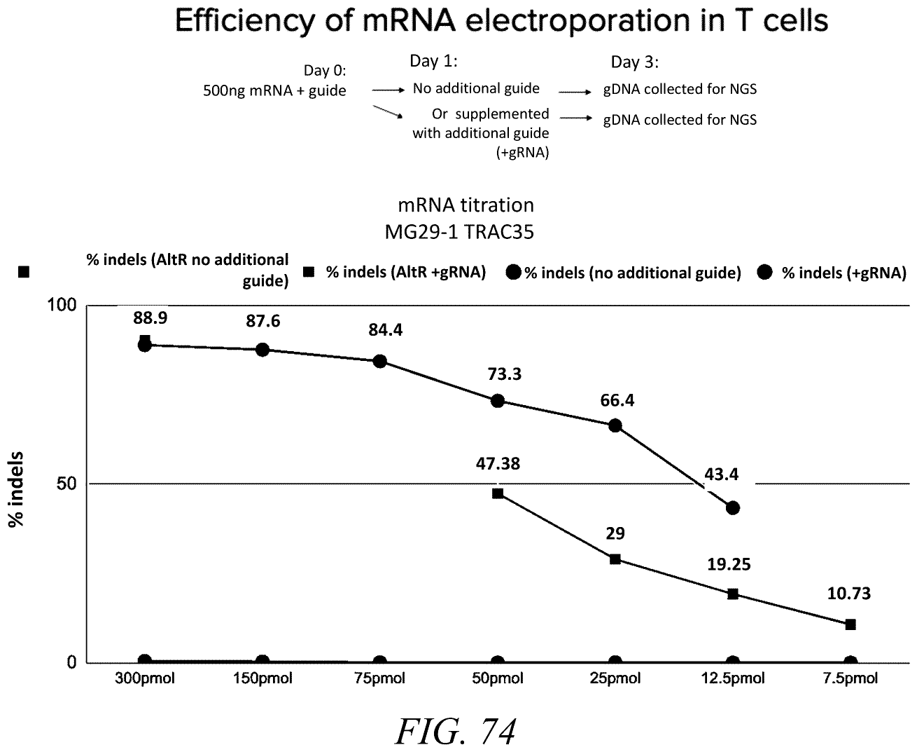

Primary T cells were purified from PMBCs using a negative selection kit (Miltenyi) according to the manufacturer's recommendations. Nucleofection of mRNA was performed as follow: 200,000 cells were co-transfected with 500 ng of mRNA and the indicated amount of guide RNA using a Lonza 4D electroporator (DS-120). Cells were harvested and genomic DNA prepared three days post initial transfection. For conditions labeled “+gRNA”: 15 h post initial transfection, cells were nucleofected with indicated amount of additional guide. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 74).

### Example 53—Editing with Chemically Modified Guides

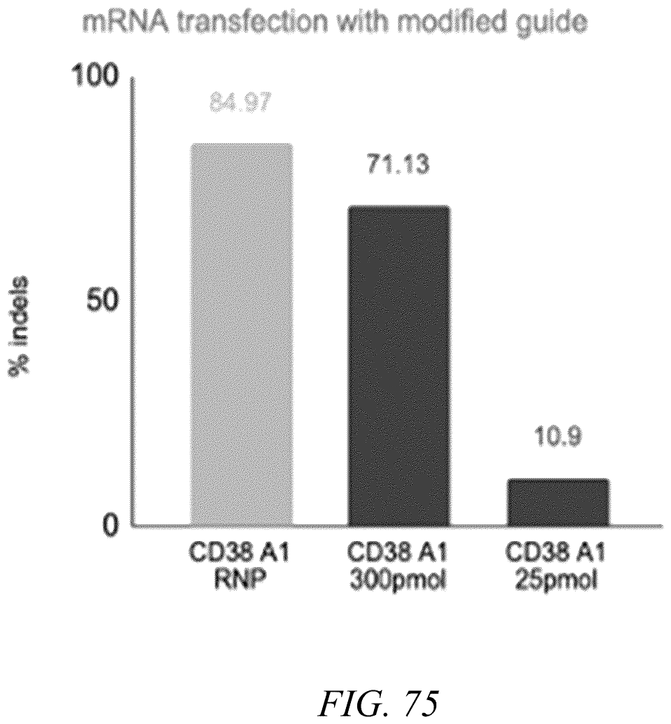

Primary T cells were purified from PBMCs using a negative selection kit (Miltenyi) according to the manufacturer's recommendations. Nucleofection of mRNA was performed as follow: 200,000 cells were co-transfected with 500 ng of mRNA and the indicated amount of guide using a Lonza 4D electroporator (DS-120). Cells were harvested and genomic DNA prepared three days post initial transfection. Nucleofection of RNPs was performed by combining 120 pmol protein and 160 pmol guide RNA. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 75).

### Example 54—ELISA Assay to Assess Pre-Existing Antibody Response

MG29-1 was expressed in and purified from human HEK293 cells using the Expi293™ Expression System Kit (ThermoFisher Scientific). Briefly, 293 cells were lipofected with plasmids encoding the nucleases driven by a strong viral promoter. Cells were grown in suspension culture with agitation and harvested two days post-transfection. The nuclease proteins were fused to a Six-His affinity tag and purified by metal-affinity chromatography to between 50-60% purity. Parallel lysates were made from mock-transfected cells and were subjected to an identical metal-affinity chromatography process. Cas9 was purchased from IDT and is >95% pure.

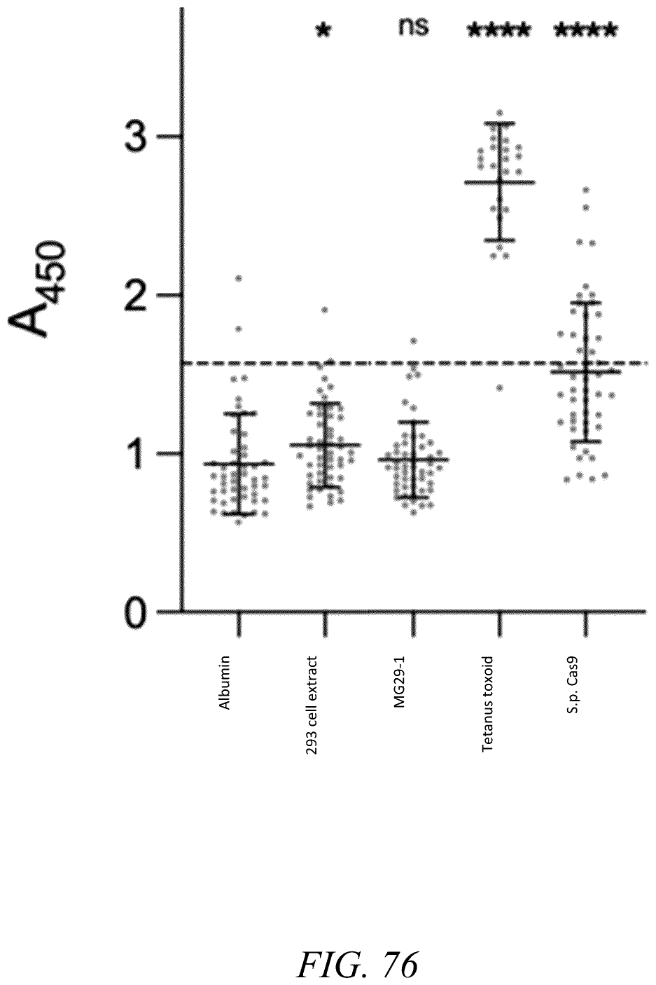

MaxiSorp® ELISA plates (Thermo Scientific) were coated with 0.5 μg of nucleases or control proteins diluted in 1× phosphate buffered saline (PBS) and incubated overnight at room temperature. Plates were then washed and incubated with a 1% (w/v) bovine serum albumin (BSA) (Sigma-Aldrich)/1× PBS solution (1% BSA-PBS) for an hour at room temperature. After another washing operation, wells were incubated for 1 h at room temperature with more than 50 separate serum samples taken from randomly selected human donors (1:50 dilution in 1% BSA-PBS). Plates were then washed and incubated for an hour at room temperature with a peroxidase-labeled goat anti-human (Fcγ fragment-specific) secondary antibody (Jackson Immuno Research), diluted 1:50,000 in 1% BSA-PBS. The assay was developed using a 3,3′,5,5′-Tetramethylbenzidine (TMB) Liquid Substrate System kit (Sigma-Aldrich), according to the manufacturer's specifications. Antibody titers are reported as absorbance values measured at 450 nm (FIG. 76). Tetanus toxoid was used as the positive control due to wide-spread vaccination against this antigen and was purchased from Sigma Aldrich. The data indicated that, in contrast to SpCas9 and tetanus toxoid (positive control), MG29-1 had similar antibody response to albumin and 293T cell extract, indicating that the donors did not have existing exposure to antigenic epitopes of MG29-1. This suggests this enzyme may be more efficacious for in vivo editing as it would be less susceptible to inactivation in vivo by existing antibody responses.

### Example 55—In Vivo Editing of Mouse HAO-1 with MG29-1 Delivered via Lipid Nanoparticle (LNP)

The human genetic disease Primary Hyperoxaluria Type I (PH1) is caused by mutations in the alanine-glyoxylate aminotransferase gene (AGXT) that disrupt glycolate metabolism in the liver and result in the overproduction of oxalate. Oxalate is an insoluble metabolite that is cleared from the body by the kidney and excreted in the urine. Elevated levels of oxalate production result in the accumulation of oxalate in the kidney and other organs which results in kidney failure as well as damage to other organs. The available curative treatment for PH1 is a liver transplant which is often combined with a kidney transplant to replace the defective kidney function. The HAO-1 gene encodes the enzyme glycolate oxidase (GO) which lies upstream of AGXT in the glycolate metabolic pathway. Reduction in the amount of GO protein reduces the production of oxalate and is thus an effective approach for the treatment of PH1 as demonstrated in a mouse model of PH1 (see Martin-Higueras et al. Molecular Therapy vol. 24 no. 4, 719-725 (2016) doi: 10.1038/mt.2015.224, which is incorporated by reference in its entirety herein) and in clinical studies with a RNAi drug that targets HAO-1 (see Frishberg et al. CJASN July 2021, 16 (7) 1025-1036 doi: 10.2215/CJN.14730920, which is incorporated by reference in its entirety herein).

A genome editing approach that knocks down the HAO-1 gene is an attractive approach for a curative therapy for PH1 patients. One approach for a genome editing therapy for PH1 is to create a double strand break within the coding region of the HAO-1 gene in hepatocytes which is repaired by the non-homologous end joining (NHEJ) DNA repair pathway. Hepatocytes are the cell type in the liver that express the HAO-1 gene and the NHEJ pathway is the dominant DNA repair pathway in these cells. The NHEJ repair pathway is error-prone and introduces insertions or deletions at the site of the double strand break which can lead to frame shifts (if the insertions or deletions are not multiples of 3 nucleotides) or to deletions or insertions of amino acids. The introduction of a frame shift can lead to the induction of nonsense-mediated mRNA decay which reduces the level of the mRNA, which further contributes to protein knockdown.

Double-strand breaks can be generated in a sequence specific manner by RNA guided CRISPR-Cas nucleases. MG29-1 is a type V CRISPR nuclease that utilizes a short guide RNA of between 38 and 42 nucleotides. MG29-1 primarily generates deletions at the cut site when tested in cultured mammalian cells which makes it attractive for the purposes of knocking down a gene. In order to be useful for in vivo therapeutics, MG29-1 activity is ideally preserved in living mammals when delivered using a clinically appropriate delivery system. Lipid nanoparticles represent an attractive delivery system for in vivo genome editing of hepatocytes in the liver because they efficiently deliver mRNA and sgRNA to hepatocytes after intravenous administration in rodents and primates. We therefore evaluated MG29-1 as a genome editing system for use in knocking down the HAO-1 gene as a potential therapy for PH1.

Messenger RNA encoding the MG29-1 nuclease was generated by in vitro transcription of a linearized plasmid template using T7 RNA polymerase and a mixture of ribonucleotides rATP, rCTP and rGTP and N1-methyl pseudouridine in place of rUTP and CleanCAP (Trilink). The plasmid also encoded an approximately 100 nt polyA tail at the 3′ end of the coding sequence. In addition to an mRNA encoding the wild type MG29-1 protein, a second mRNA encoding the MG29-1 protein with a single amino acid change of S168R was synthesized. The mRNA was purified on commercial spin columns, the concentration was determined by absorbance at 260 nM, and the purity was determined by Tape Station (Agilent).

Guide RNAs were selected based on editing efficiency evaluations of multiple guides spanning exons 1 to 4 of the mouse HAO-1 gene in the mouse liver cell line Hepa1-6. Three guides were chemically synthesized incorporating a combination of chemical modifications of bases at specific positions referred to collectively as chemical modification #37; these guide RNAs are shown below in Table 25.

TABLE 25
Sequences and chemical modifications of
guide RNA tested in vivo in mice

| guide RNA | SEQ | |
|---|---|---|
| Name | ID No. | Sequence |
| mH29-1 37 | 5779 | mC*mU*mU*U*UAAUUmUmC |
| | | mUmACU*G*U*U*GUAGAUC |
| | | CCCAGAfCfCfUfGfUfAfA |
| | | fUfAfG*fU*fCfA*fU*mA |
| mH29-15 37 | 5780 | mC*mU*mU*U*UAAUUmUmC |
| | | mUmACU*G*U*U*GUAGAUU |
| | | GACUGUfGfGfAfCfAfCfC |
| | | fCfCfU*fU*fAfC*fC*mU |
| mH29-29 37 | 5781 | mC*mU*mU*U*UAAUUmUmC |
| | | mUmACU*G*U*U*GUAGAUC |
| | | CUUAGGfAfGfAfAfAfAfU |
| | | fGfCfC*fA*fAfA*fU*mC |

Code for modified bases and linkages: m: 2′-O-methyl modified base, f: 2′-fluoro modifed base, *: phosphorothioate linkage

The MG29-1 mRNA and the guide RNA were separately packaged inside lipid nanoparticles (LNP) using a process essentially as described by Kaufmann et al (Nano Lett. 2015, 15, 11, 7300-7306, PMID: 26469188, DOI:10.1021/acs.nanolett.5b02497, which is incorporated by reference herein in its entirety). Lipids were purchased from Avanti Polar Lipids or from Corden Pharma and dissolved in ethanol. The mRNA or sgRNA was prepared in water then diluted in 100 mM sodium acetate (pH 4.0) to make the RNA working stock. The four lipid components were combined in ethanol at the specific ratios to make the lipid working stock. An example lipid mixture comprised cholesterol, 1,2-dioleoyl-sn-glycero-3-phosphoethanolamine (DOPE), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), and DMG-PEG-2000 at molar ratios of 47.5:16:35:1.5. The lipid working stock and the RNA working stock were combined in a microfluidics mixing device (Precision Nanosystems) at a flow rate of 12 mL/min and a ratio of 1 volume of lipid working stock to 3 volumes of RNA working stock. The mass ratio of C12-200 to RNA in the formulation was 10 to 1. The formulated LNPs were diluted 1:1 with 1× PBS then dialyzed twice in 1× PBS for 1 hour each followed by concentration in Amicon spin concentrators. The resultant LNPs were formulated in 1× PBS buffer, filter sterilized through a 0.2 uM filter, and stored at 4° C. The concentration of the RNA inside and outside of the LNP was measured using the Ribogreen reagent (Thermo Fisher). The average diameter and polydispersity of the LNPs were measured in the resultant concentrated LNP by dynamic light scattering using a NanoBrook 90Plus (Brookhaven Instruments).

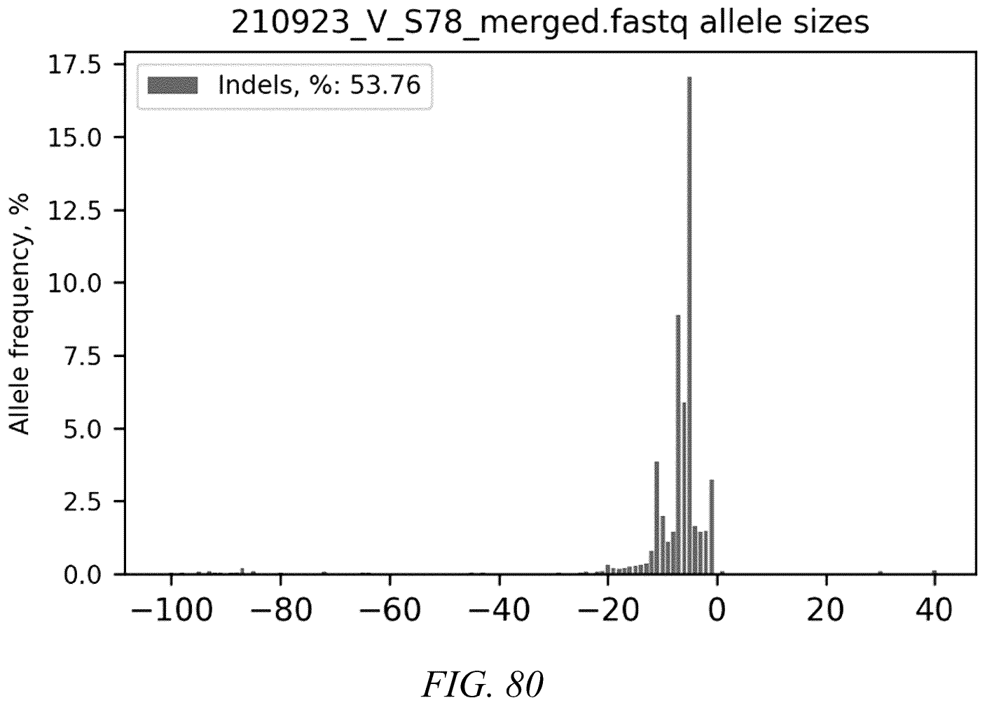

LNPs encapsulating a guide RNA and the MG29-1 mRNA or MG29-1_S168R mRNA were mixed at a RNA mass ratio of 1:1, then injected intravenously into wild type C57Bl/6 mice via the tail vein at a dose of 1 mg of RNA per kg in a total volume of 0.1 ml per mouse. Mice were sacrificed at 10 days post dosing and the 3 lobes of the liver (left lateral, right lateral, medial) were collected, flash frozen, and stored at −80° C. The entire left lateral lobe of the liver was homogenized in Genomic Digestion Buffer (Purelink Genomic DNA Purification Kit, Thermo Fisher) using 0.4 mL of buffer per 100 mg of tissue weight in a Bead Mill. Genomic DNA was purified from an aliquot of the homogenate using the Purelink Genomic DNA Purification Kit (Thermo Fisher). The region of the HAO-1 gene targeted by each specific guide RNA was PCR amplified using gene specific primers with adapters complementary to the barcoded primers used for next generation sequencing (NGS) in a PCR reaction comprised of the Q5 high fidelity DNA polymerase and a total of 29 cycles. The product of this first PCR reaction was PCR amplified using the barcoded primers for NGS using a total of 10 cycles. The resulting product was subjected to NGS on an Illumina MiSeq instrument and the results were processed using a custom script to generate the percentage of sequencing reads that contain insertions or deletions (INDELS) at the targeted site in the HAO-1 gene. The genomic DNA from livers of mice injected with PBS buffer were used as controls. The average sequencing read count was 142,000 reads (range 54,000 to 205,000). The NGS data also enabled a prediction of the percentage of INDELS that generate a frame shift as well as a determination of the INDEL profile (FIG. 80).

Total protein was extracted from the entire right lateral lobe of the liver from the same mice by homogenization in PBS in a bead mill followed by three rounds of freeze-thaw at −80° C. and room temperature. The lysate was centrifuged to remove tissue debris and the supernatant was collected. The concentration of total protein in the supernatant was determined using the BCA assay. Equal amounts of total protein were fractionated on SDS-PAGE gels and transferred to nitrocellulose membranes which were then probed with an anti-glycolate oxidase antibody (R&D Systems AF6197, sheep anti-HAO1). Detection utilized an HRP-conjugated secondary antibody (R&D Systems HAF016, Donkey anti-Sheep IgG) followed by detection with SuperSignal West Dura Chemiluminescent substrate (ThermoFisher Cat. #34076) and visualization with the Bio-Rad ChemiDoc MP imager.

Editing activity of the tested guides is summarized below in Table 26.

TABLE 26
Editing activity of guide RNA tested in vivo in mice

| | | Editing activity in | Editing activity in |
|---|---|---|---|
| guide | Exon of | Hepa1-6 cells (% | Hepa1-6 cells (% |
| RNA | HAO-1 | INDELS) by mRNA | INDELS) by RNP |
| name | targeted | transfection | nucleofection |
| mH29-1_37 | Exon 1 | 17% | 92.6% |
| mH29-15_37 | Exon 3 | 34% | 96.8% |
| mH29-29_37 | Exon 4 | 35% | 93.8% |

By nucleofection of ribonuclear protein complexes, these guide RNA all had editing activity of 90% or greater in Hepa1-6 cells. The level of editing in Hepa1-6 cells when the MG29-1 mRNA and the guide RNA were co-transfected using lipofection (MessengerMax reagent, Thermo Fisher) was lower due to the lower transfection efficiency, and guides mH29-15 and mH29-29 were significantly more potent than mH29-1 when tested by this transfection method.

The characteristics of the LNP encapsulating MG29-1 mRNA and the guide RNA are summarized in Table 27 below.

TABLE 27
Characteristics of lipid nanoparticles (LNP)

| | | LNP | LNP | % Recovery | % of RNA |
|---|---|---|---|---|---|
| LNP | Payload | Diameter | PDI | of RNA (yield) | Encapsulated |
| L012-A | mH29-1_37 | 74 | 0.15 | 71 | 92.7 |
| L012-B | mH29-15_37 | 85 | 0.08 | 87 | 93.5 |
| L012-C | mH29-29_37 | 83 | 0.121 | 78 | 92.1 |
| L012-M | MG29-1 mRNA | 98 | 0.13 | 97 | 92.9 |
| L012-S | MG29-1_S168R mRNA | 76 | 0.10 | 84 | 92.8 |

The LNP encapsulating the 3 guide RNAs had diameters between 74 nm and 85 nm with polydispersity (PDI) of 0.08 to 0.15. The LNP encapsulating the MG29-1 mRNA and the MG29-1_S168R mRNA had diameters of 98 nm and 76 nm, respectively, and PDI values less than 0.15 indicative of low polydispersity. The percentage of input RNA recovered in the resultant LNP ranged from 71% to 97% and the percentage of the total RNA that was encapsulated inside the LNP was 92% or greater for all the LNPs. These data demonstrate that both the guide RNA and the MG29-1 mRNA can be efficiently encapsulated in LNPs and that the resulting LNPs were of small size (less than 100 nM) and low polydispersity.

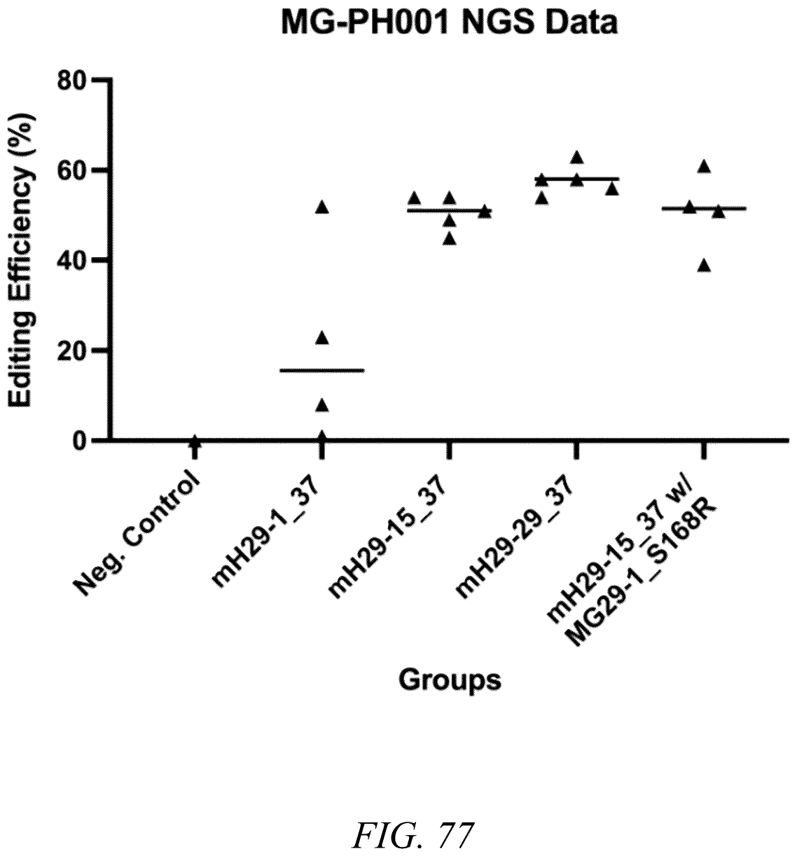

The level of editing at the target site in the HAO-1 gene 10 days after intravenous injection of LNP encapsulating MG29-1 mRNA and each of the guide RNAs is shown in FIG. 77. As expected, a mouse injected with PBS buffer had no editing. Mice injected with LNP encapsulating MG29-1 mRNA and the guide RNA mH29-1_37 exhibited variable levels of editing that ranged from 1% to 52%. Mice injected with LNP encapsulating MG29-1 mRNA and the guide RNA mH29-15_37 exhibited consistent levels of editing with a mean of 50.4% (range 45% to 54%). Mice injected with LNP encapsulating MG29-1 mRNA and the guide RNA mH29-29_37 exhibited consistent levels of editing with a mean of 57.7% (range 54% to 63%). Mice injected with LNP encapsulating MG29-1_S168R mRNA and the guide RNA mH29-15_37 exhibited consistent levels of editing with a mean of 50.8% (range 38% to 61%). These results demonstrate that mRNA encoding the MG29-1 nuclease together with a guide RNA with an optimized chemistry can edit a target locus in the liver of mice when delivered in an LNP. Among the 3 guide RNAs with different spacer sequences that were tested, two exhibited consistently high levels of editing while one exhibited more variable editing. Because LNP of this type deliver their payload almost exclusively to hepatocytes, and because hepatocytes make up 60 to 65% of the total number of cells in the rat liver (Bale et al, Scientific Reports volume 6, Article number: 25329 (2016) doi: 10.1038/srep25329, which is incorporated by reference in its entirety herein) and 52% of the total cells in the mouse liver (Barratta et al, Histochemistry and Cell Biology volume 131, pages 713-726 (2009) doi: 10.1007/s00418-009-0577-1, which is incorporated by reference in its entirety herein), the maximum level of editing achievable if every hepatocyte were edited at each copy of the HAO-1 gene is predicted to be 60% to 65%. Thus, 50% editing measured in total genomic DNA purified from the liver represents editing in approximately 75 to 80% of the hepatocytes. The inclusion of the S168R amino acid change in MG29-1, which can improve editing efficiency in cultured cells, did not improve editing efficiency in this study with the one guide RNA tested. The improvement in editing efficiencies with the S168R amino acid variant of MG29-1 was previously observed with guide RNAs that exhibited low editing, which may explain why no improvement was observed here with a guide RNA that was already selected for high levels of editing. The INDEL profile (FIG. 80) was composed entirely of deletions. The majority of the deletions were between 1 and 11 nucleotides with a small number of larger deletions. The frequency of predicted frame shift creation among the mice treated with guides mH29-15 and mH29-29 ranged from 70 to 80% of the total INDELS with a mean of 75%. Thus, on average, 75% of the observed INDELS are predicted to create a frame shift in the HAO-1 coding sequence which will result in disruption of the amino acid sequence downstream of the editing site and a high chance of creating a stop codon.

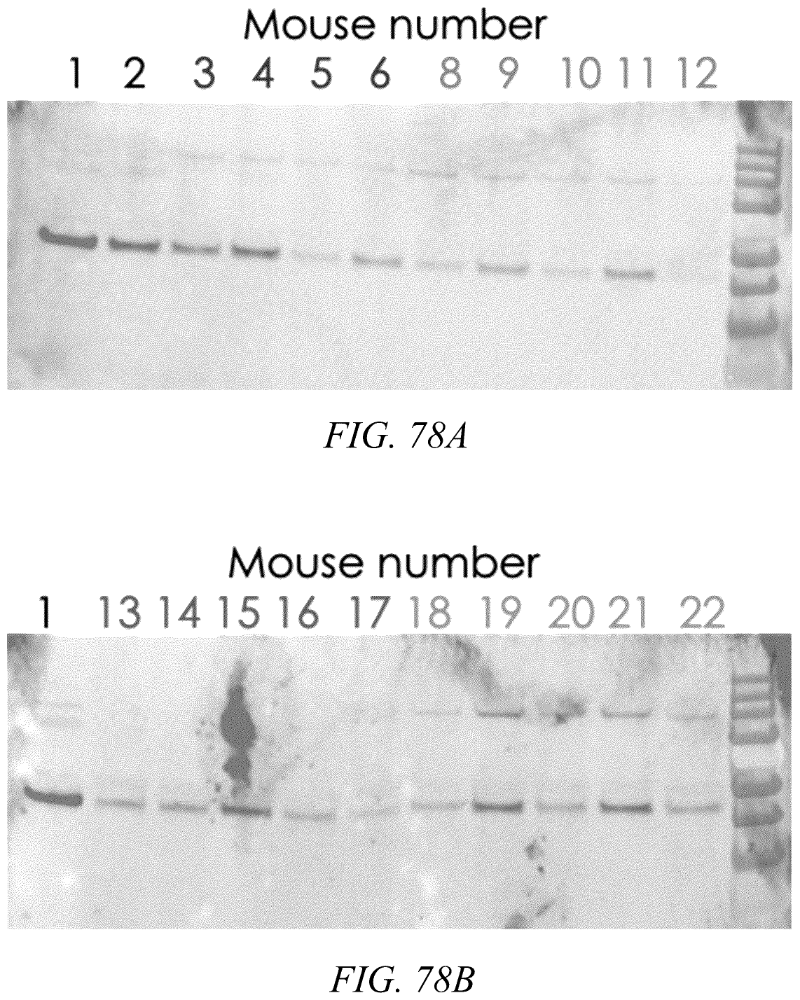

The other main lobe of the liver from the same mice was analyzed for the level of the protein glycolate oxidase (GO) that is the product of the HAO-1 gene to determine if the INDELS introduced into the HAO-1 gene resulted in a reduction in GO protein levels in the liver. Western blot analysis using an antibody to the GO protein detected a band of the expected size (Table 28 and FIGS. 78A-B).

TABLE 28
Editing of glycolate oxidase in mice

| Mouse | Treatment | Editing % |
|---|---|---|
| 1 | PBS | 0 |
| 2 | | 0 |
| 3 | mH29-1_37 | 23 |
| 4 | MG29-1_mRNA | 1 |
| 5 | | 52 |
| 6 | | ND |
| 8 | mH29-15_37 | 54 |
| 9 | MG29-1_mRNA | 54 |
| 10 | | 49 |
| 11 | | 45 |
| 12 | | 51 |
| 13 | mH29-29_37 | 56 |
| 14 | MG29-1_mRNA | 63 |
| 15 | | 58 |
| 16 | | 58 |
| 17 | | 54 |
| 18 | mH29-15_37 | 61 |
| 19 | MG29-1 S168R_mRNA | 39 |
| 20 | | 51 |
| 21 | | 52 |
| 22 | | ND |

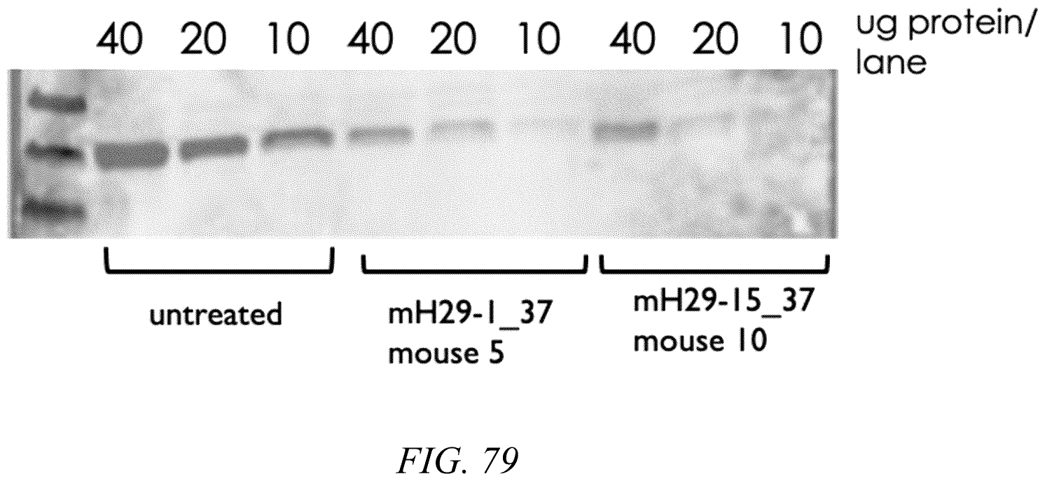

In comparison to mice that received PBS buffer, mice that received LNP encapsulating MG29-1 mRNA and the various guide RNAs exhibited reduced levels of the GO protein. In general, the magnitude of the reduction in GO protein correlated to the editing efficiency at the HAO-1 gene as measured in the same mouse by NGS. Liver protein from 2 of the mice that showed reductions in GO protein were further tested by loading 3 different amounts of total protein on the gel and repeating the Western blot. As shown in FIG. 79, the reduction of GO protein in the 2 mice treated with LNP encapsulating MG29-1 mRNA and either guide RNA mH29-1_37 or mH29-15_37 was clearly observed at the different loadings of total protein. These data demonstrate that the editing of the HAO-1 gene resulted in reductions in the level of GO protein in the liver of the mice.

### Example 56—Gene Editing Outcomes at the DNA Level for TRAC in Human Peripheral Blood B Cells

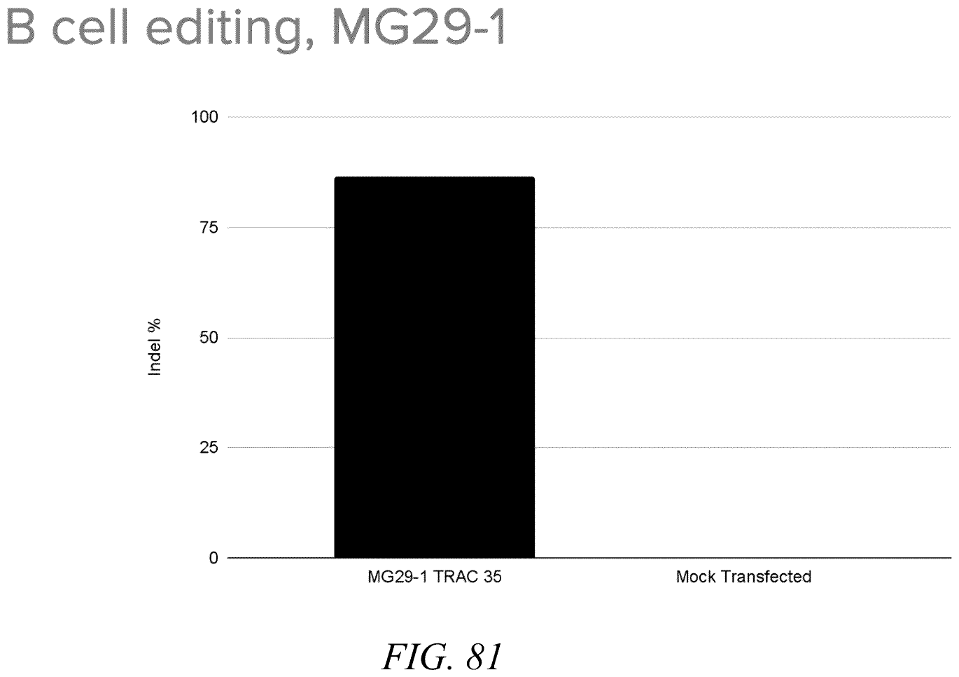

Human Peripheral Blood B cells were purchased from STEMCELL Technologies and expanded using ImmunoCult™ Human B Cell Expansion Kit for 2 days prior to nucleofection. Nucleofection of MG29-1 RNPs (126 pmol protein/160 pmol guide) was performed into B cells (200,000) using the Lonza 4D electroporator. Cells were harvested and genomic DNA prepared three days post-transfection. For NGS analysis PCR primers appropriate for use in NGS-based DNA sequencing were used to amplify the target sequence for the TRAC 35 guide RNA (SEQ ID NO: 5681). The amplicon was sequenced on an Illumina MiSeq machine and analyzed with a proprietary Python script to measure gene editing (FIG. 81).

TABLE 28B
Sequences of Guide RNAs and Sequences
Targeted for Example 56

| | SEQ | | |
|---|---|---|---|
| Guide | ID | Guide | |
| Target | NO | Name | SEQUENCE |
| MG29-1 | 5681 | MG29-1- | mU*rArArUrUrUrCrUrArCrUr |
| sgRNA | | TRAC- | GrUrUrGrUrArGrArUrGrArGr |
| target- | | sgRNA-35 | UrCrUrCrUrCrArGrCrUrGrGr |
| ing | | | UrArCrArCrG*mG |
| TRAC | | | |
| DNA | 5682 | MG29-1- | GAGTCTCTCAGCTGGTACACGG |
| Sequence | | TRAC- | |
| of | | Target | |
| TRAC | | site-35 | |
| target | | | |
| site | | | |
| MG29-1 | 5683 | MG29-1- | /AltR1/rUrArArUrUrUrCrUr |
| sgRNA | | TRAC- | ArCrUrGrUrUrGrUrArGrArUr |
| target- | | sgRNA-35- | GrArGrUrCrUrCrUrCrArGrCr |
| ing | | AltR | UrGrGrUrArCrArCrGrG/ |
| TRAC | | | AltR2/ |
| DNA | 5684 | MG29-1- | GAGTCTCTCAGCTGGTACACGG |
| sequence | | TRAC- | |
| of | | target | |
| TRAC | | site- | |
| target | | 35-AltR | |
| site | | | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 57—Gene Editing Outcomes at the DNA Level for TRAC in Hematopoietic Stem Cells (HSCs)

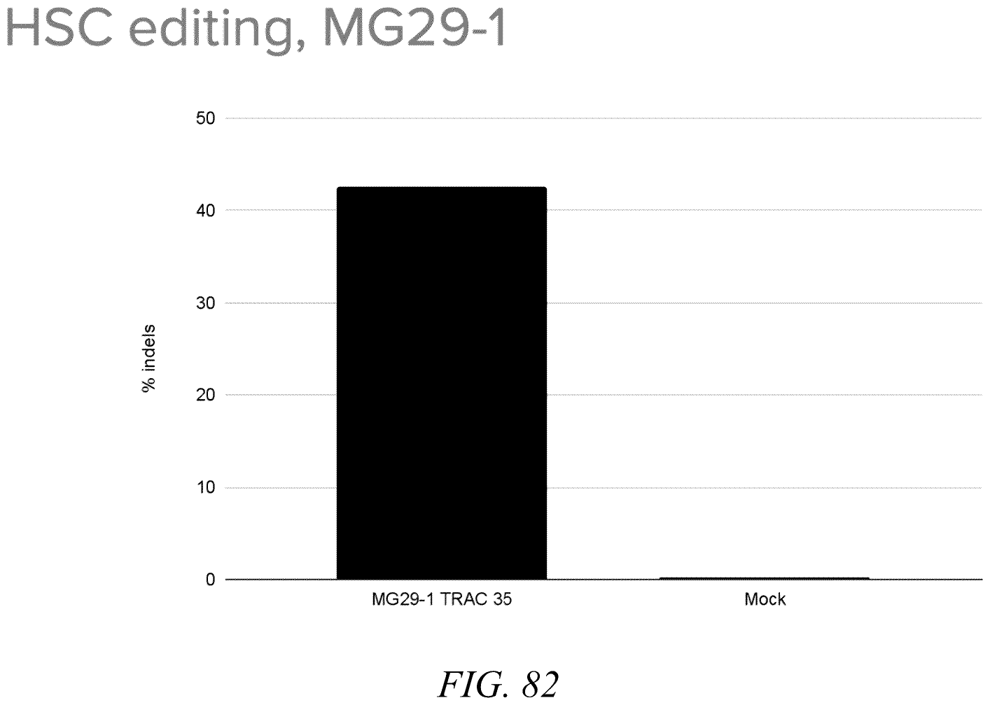

Mobilized peripheral blood CD34+ cells were acquired from AllCells and cultured in STEMCELL StemSpan™ SFEM II media supplemented with StemSpan™ CC110 cytokine cocktail for 48 hours prior to nucleofection. Nucleofection of MG29-1 RNPs (126 pmol protein/160 pmol guide) was performed into HSCs (200,000) using the Lonza 4D electroporator. Cells were harvested and genomic DNA prepared three days post-transfection. PCR primers appropriate for use in NGS-based DNA sequencing used to amplify the individual target sequences for MG29-1 TRAC 35 gRNA (SEQ ID NO: 5681). The NGS amplicons were sequenced on an Illumina MiSeq machine and analyzed with a proprietary Python script to measure gene editing (FIG. 82).

### Example 58—Gene Editing Outcomes at the DNA Level for TRAC in Induced Pluripotent Stem Cells (iPSCs)

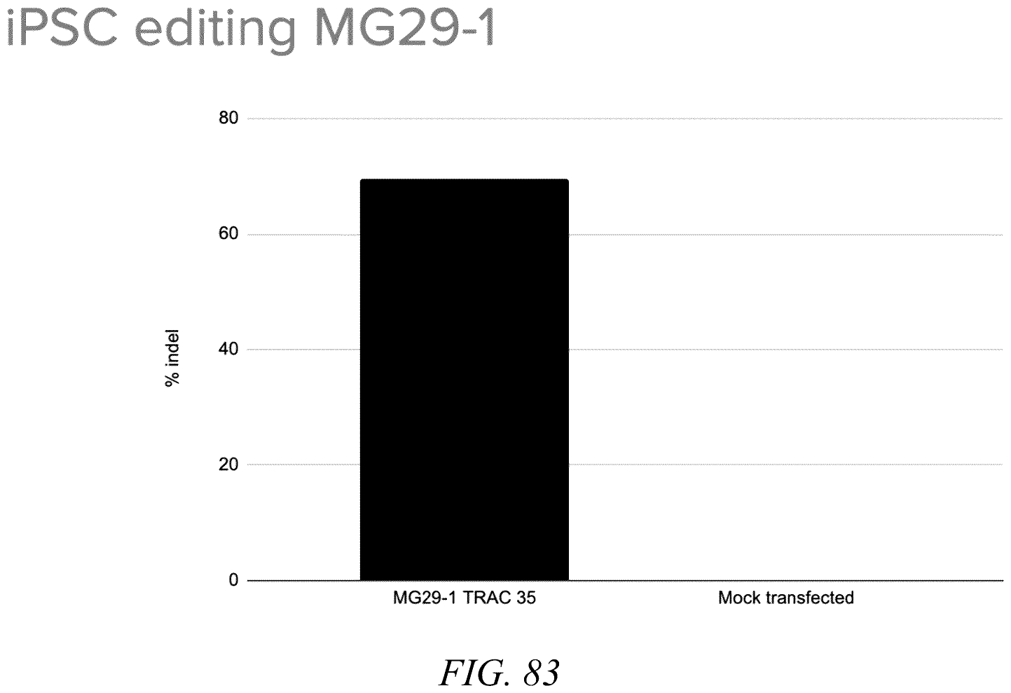

ATCC-BXS0116 Human [Non-Hispanic Caucasian Female] Induced Pluripotent Stem (IPS) Cells are cultured on Corning Matrigel-coated plasticware in mTESR Plus (STEMCELL Technologies) containing 10 μM ROCK inhibitor Y-27632 for 24 hr prior to nucleofection. Nucleofection of MG29-1 RNP (126 pmol protein/160 pmol guide) was performed into iPSCs (200,000) using the Lonza 4D electroporator. Cells were harvested with Accutase for genomic DNA extraction five days post-transfection. PCR primers appropriate for use in NGS-based DNA sequencing were used to amplify the individual target sequences for the TRAC 35 gRNA (SEQ ID NO: 5681). The amplicons were sequenced on an Illumina MiSeq machine and analyzed with a proprietary Python script to measure gene editing (FIG. 83).

### Example 59—In Vivo Genome Editing with the MG29-1 Nuclease Quantified by Next Generation Sequencing (NGS)

To evaluate the ability of the MG29-1 Type V nuclease to edit the genome in vivo in a living animal, an mRNA encoding the MG29-1 nuclease and one of four guide RNAs were delivered in a lipid nanoparticle. The four guide RNAs tested were mA1b298-37, mA1b2912-37, mA1b2918-37, and mA1b298-34, the sequences of which are shown below in Table 29. Guides mA1b298-37 and mA1b298-34 have the same nucleotide sequence but different chemical modifications, while guides mA1b298-37, mA1b2912-37, and mA1b2918-37 have different spacer sequences but the same chemical modifications.

Sequences and chemical modifications of
guide RNA tested in vivo in mice

| | SEQ | |
|---|---|---|
| | ID | |
| Guide name | NO. | Sequence |
| mAlb298-37 | 5756 | mC*mU*mU*U*UAAUUmUmC |
| | | mUmACU*G*U*U*GUAGAUC |
| | | UGUAACfGfAfUfCfGfGfG |
| | | fAfAfC*fU*fGfG*fC*mA |
| mAlb2912-37 | 5757 | mC*mU*mU*U*UAAUUmUmC |
| | | mUmACU*G*U*U*GUAGAUA |
| | | GUGUAGfCfAfGfAfGfAfG |
| | | fGfAfA*fC*fCfA*fU*mU |
| mAlb2918-37 | 5758 | mC*mU*mU*U*UAAUUmUmC |
| | | mUmACU*G*U*U*GUAGAUA |
| | | AGAUUGfAfUfGfAfAfGfA |
| | | fCfAfA*fC*fUfA*fA*mC |
| mAlb298-34 | 5759 | mC*rU*rUrArArUrUmUmC |
| | | mUmArCrUrGrUrUrGmUmA |
| | | mGmArUrCrUrGrUrArArC |
| | | rGrArUrCrGrGrGrA*rAf |
| | | C*fUfG*fGfC*mA |

M: 2′-O methyl modified base; f: 2′-fluorine modified base; *: phosphorothioate backbone

In an in vitro stability assay in Hepa1-6 cell lysates, the mA1b298-37 guide was more stable than the mA1b298-34 guide, demonstrating that the chemical modifications on the mA1b298-37 guide were more effective at protecting the guide RNA against degradation.

The mRNA encoding MG29-1 (SEQ ID NO: 5687) was generated by in vitro transcription of a linearized plasmid template using T7 RNA polymerase using nucleotides and enzymes purchased from New England Biolabs or Trilink Biotechnologies. The protein coding sequence of the MG29-1 cassette comprises the following elements from 5′ to 3′: the nuclear localization signal from SV40, a five amino acid linker (GGGS), the protein coding sequence of the MG29-1 nuclease from which the initiating methionine codon was removed, a 3 amino acid linker (SGG), and the nuclear localization signal from nucleoplasmin. The DNA sequence of this cassette was codon optimized for human using a commercially available algorithm. An approximately 100 nucleotide polyA tail was encoded in the plasmid used for in vitro transcription and the mRNA was co-transcriptionally capped using the CleanCAP (™) reagent purchased from Trilink Biotechnologies. Uridine in the mRNA was replaced with N1-methyl pseudouridine.

To generate the lipid nanoparticle (LNP) formulation used to deliver the MG29-1 mRNA and the guide RNA, the four lipid components were dissolved in ethanol and mixed in an appropriate molar ratio to make the lipid working mix. The mRNA and the guide RNA were either mixed prior to formulation at a 1:1 mass ratio or formulated in separate LNP that were later co-injected into mice at a 1:1 mass ratio of the two RNA's. In either case, the RNA was diluted in 100 mM Sodium Acetate (pH 4.0) to make the RNA working stock. The lipid working stock and the RNA working stock were mixed in a microfluidics device (Ignite NanoAssembler, Precision Nanosystems) at a flow rate ratio of 1:3, respectively, and a flow rate of 12 mL/min. The LNP were dialyzed against phosphate buffered saline (PBS) for 2 to 16 hours and then concentrated using Amicon spin concentrators (Milipore) until the pre-determined volume was achieved. The concentration of RNA in the LNP formulation was measured using the Ribogreen reagent (Thermo Fisher). The diameter and polydispersity (PDI) of the LNP were determined by dynamic light scattering. Example LNP had diameters ranging from 65 nm to 120 nm with PDI of 0.05 to 0.20. LNP were injected intravenously into 8 to 12 week old C57B16 wild type mice via the tail vein (0.1 mL per mouse) at a total RNA dose of 1 mg RNA per kg body weight. Three days post dosing, the mice were sacrificed and the liver was collected and homogenized using a bead beater (Omni International) in a digestion buffer supplied in the PureLink Genomic DNA Isolation Kit (Thermo Fisher Scientific). Genomic DNA was purified from the resulting homogenate using the PureLink Genomic DNA Isolation Kit (Thermo Fisher Scientific) and quantified by measuring the absorbance at 260 nm. Genomic DNA purified from mice injected with buffer alone was used as a control.

The liver genomic DNA was then PCR amplified using a first set of primers flanking the region targeted by the guides. The PCR primers used are shown below in Table 30. The 5′ end of these primers comprise conserved regions complementary to the PCR primers used in the second PCR, followed by 5 Ns in order to give sequence diversity and improve MiSeq sequencing quality, and end with sequences complementary to the target region in the mouse genome. PCR was performed using Q5@ Hot Start High-Fidelity 2× Master Mix (New England Biolabs) on 100 ng of genomic DNA and an annealing temperature of 60° C. for a total of 30 cycl^{es}. This was followed by a 2nd round of 10 cycles of PCR using primers designed to add unique dual Illumina barcodes (IDT) for next generation sequencing on a MiSeq instrument. Each sample was sequenced to a depth of greater than 10,000 reads using 150 bp paired end reads. Reads were merged to generate a single 250 bp sequence from which Indel percentage and INDEL profile was calculated using a proprietary Python Script.

TABLE 30
Sequences of PCR primers and Next Generation Sequencing primers used to
analyze in vivo genome editing in mice

| | | SEQ | |
|---|---|---|---|
| Primer name | Purpose | ID NO. | Sequence |
| mAlb298_NGS_F | Amplify the target | 5782 | GCTCTTCCGATCTNNNNNCTTG |
| | site in albumin | | AGTTTGAATGCACAGATA |
| | intron 1 | | |
| mAlb298_NGS_R | Amplify the target | 5783 | GCTCTTCCGATCTNNNNNTGG |
| | site in albumin | | AAACAGGGAGAGAAAAACC |
| | intron 1 | | |
| mAlb2912_NGS_F | Amplify the target | 5784 | GCTCTTCCGATCTNNNNNTACA |
| | site in albumin | | AACATGACAGAAACACTAA |
| | intron 1 | | |
| mAlb2912_NGS_R | Amplify the target | 5785 | GCTCTTCCGATCTNNNNNGATT |
| | site in albumin | | GATGAAGACAACTAACTGT |
| | intron 1 | | |
| mAlb2918_NGS_F | Amplify the target | 5786 | GCTCTTCCGATCTNNNNNCTTT |
| | site in albumin | | GAGTGTAGCAGAGAGG |
| | intron 1 | | |
| mAlb2918_NGS_R | Amplify the target | 5787 | GCTCTTCCGATCTNNNNNCATT |
| intron 1 | site in albumin | | ATACCGATGGGCGATC |

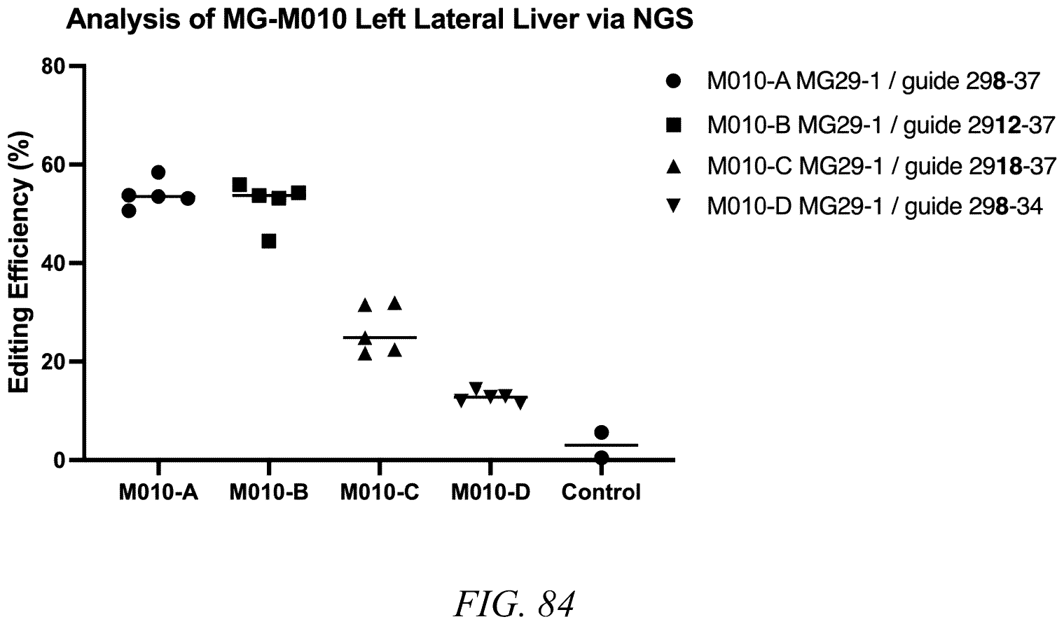

The results of the NGS analysis are shown in FIG. 84 and Table 31. Group A mice received LNP encapsulating guide RNA mASb298-37. Group B mice received LNP encapsulating guide RNA mA1b2912-37. Group C mice received LNP encapsulating guide RNA mA1b2918-37. Group D mice received LNP encapsulating guide RNA mA1b298-34. All mice in groups A to D also received LNP encapsulating the MG29-1 mRNA that was mixed with the guide RNA containing LNP at a 1:1 RNA mass ratio prior to injection. Two mice were injected with PBS as controls (Group E).

TABLE 31
Gene editing at the on target site in the liver of mice at 3 days after IV
injection of nuclease mRNA and guide RNA packaged in LNP

| | | | | Standard |
|---|---|---|---|---|
| | | Editing | | deviation |
| | | Efficiency (%) | Mean per | per |
| Group | Animal | by NGS | group | group |
| A | 1281 | 50.65 | 53.9 | 2.8 |
| | 1282 | 53.80 | | |
| | 1283 | 53.50 | | |
| | 1284 | 58.43 | | |
| | 1285 | 53.14 | | |
| B | 1286 | 53.19 | 52.3 | 4.5 |
| | 1287 | 44.49 | | |
| | 1288 | 53.73 | | |
| | 1289 | 55.90 | | |
| | 1290 | 54.27 | | |
| C | 1291 | 22.46 | 26.5 | 4.9 |
| | 1292 | 31.98 | | |
| | 1293 | 31.59 | | |
| | 1294 | 21.74 | | |
| | 1295 | 24.88 | | |
| D | 1296 | 12.94 | 12.7 | 1.1 |
| | 1297 | 14.35 | | |
| | 1298 | 11.98 | | |
| | 1299 | 12.78 | | |
| | 1300 | 11.52 | | |
| E | 1209 | 5.6 | | |
| | 1209 | 0.45 | | |

The average INDEL frequency in group A that received guide mA1b298-37 was 53.9%. The average INDEL frequency in group B that received guide mA1b2912-37 was 52.3%. The average INDEL frequency in group C that received guide mA1b2918-37 was 26.5%. The average INDEL frequency in group D that received guide mA1b298-34 was 12.7%. These data demonstrate that the MG29-1 nuclease together with a guide RNA comprised of chemical modified bases (chemistry #37 or chemistry #34) was active in vivo in the liver of mice. Guide mA1b298-34, which resulted in about 50% of the editing as guide mA1b298-37, has the same nucleotide sequence as mA1b298-37 but different chemical modifications, demonstrating that chemical modifications #37 enable significantly more editing activity in vivo than chemical modifications #34. The improved in vivo editing observed with chemistry #37 compared to chemistry #34 is consistent with the superior in vitro stability of chemistry #37.

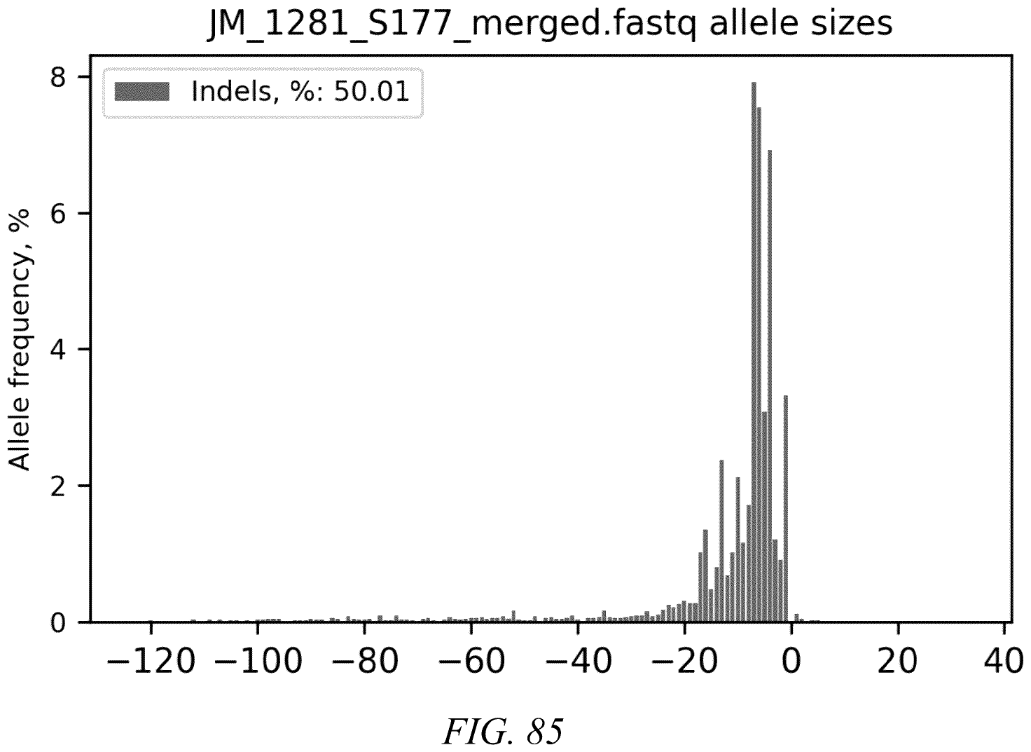

An example INDEL profile generated by the MG29-1 nuclease and guide 298-37 as measured by NGS is shown in FIG. 85. The INDEL profiles from the other 4 mice treated with the same LNP were essentially identical. The majority of the INDELS were deletions, with very few insertions detectable. This INDEL profile is distinct from that seen with spCas9, which commonly generates a mixture of insertions and deletions with a tendency to generate +1 and −1 INDELS. The deletions resulting from in vivo cleavage by MG29-1 range from −1 to −30, with the majority of deletions between −1 and −10 nucleotides.

### Example 59—Spacer Length Optimization for MG29-1 Single Guide RNA

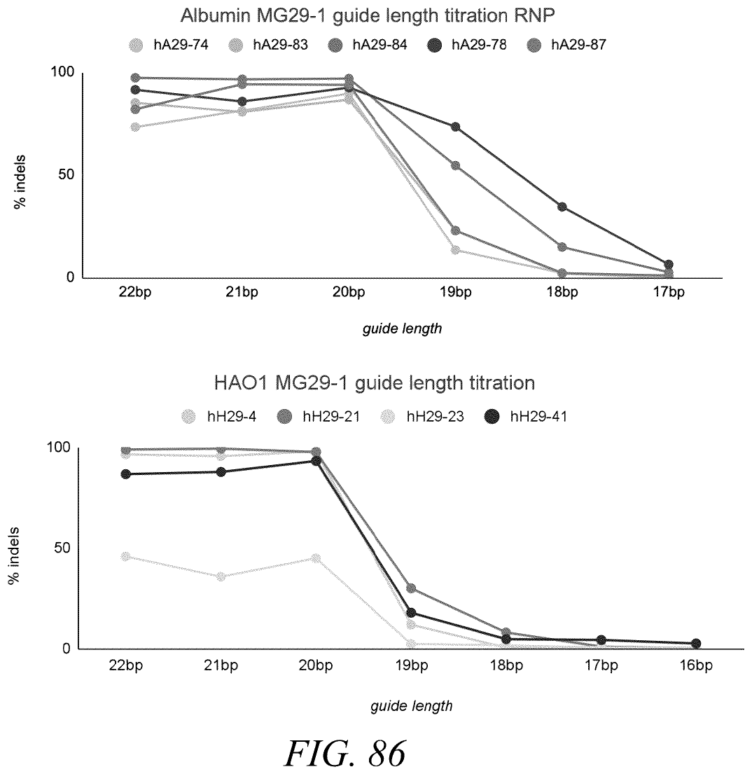

The guides tested comprised 5 different spacers targeting different regions of human albumin intron 1 (spacers 74, 83, 84, 78, and 87) with chemical modifications called “A1tR1/A1tR2” provided by Integrated DNA Technologies. The spacer length was titrated from 22 nucleotides (nt) to 17 nt by removal of nucleotides from the 3′ end of the guide RNA. Each of these guides (6 per spacer sequence) were evaluated for their editing efficiency in the human liver cell line Hep3B. Hep3B cells (1×10⁵ cells/sample) were electroporated using an Amaxa nucleofection device and program EH-100 with pre-formed ribonucleoprotein complex made by mixing 120 pmol MG29-1 protein and 160 pmol guide RNA. After electroporation, the cells were plated in 24 well plates and cultured for 3 days after which genomic DNA was purified from the cells using a commercial kit (Purelink, Invitrogen). The genomic DNA was analyzed for editing at the on target site (human albumin intron 1) by next generation sequencing (NGS). The NGS data was analyzed by a custom Python script (IndelCalculator v1.3.1). As shown in FIG. 86, editing activity was unchanged for all 5 guides when the spacer length was titrated from 22 nucleotides to 20 nucleotides. Shortening the spacer to 19 nucleotides reduced the editing activity of all 5 guides, with more pronounced 75% reductions in activity for 3 of the 5 guides. Reduction of the spacer length to 18 or 17 nucleotides further reduced the editing activity such that a 17 nucleotide spacer was inactive for all 5 guides. Similar data were obtained for 4 different guide RNA targeting a different genomic locus, the human HAO-1 gene (FIG. 86). In the case of the HAO-1 guides, editing activity dropped by 75 to 95% when the guide length was reduced from 20 nt to 22 nt. These data demonstrate that across a range of different guide spacer sequences targeting different genomic loci, the minimal MG29-1 spacer length that retained maximal editing activity was 20 nt. Because longer spacer sequences may increase the risk of off-target editing, identification of the shortest spacers that retain full activity is beneficial.

A guide RNA (“guide 29”) was identified as a highly active guide in a screen for guides with 22 nt spacers for MG29-1 that target the human HAO-1 locus. The spacer length of this guide was reduced to 20 nt by removing the 3′ most 2 nt from the spacer to create guides designated as mH29-29.1_37 (SEQ ID NO: 5710) and mH29-29.2_37 (SEQ ID NO: 5711) which differ in their chemical modifications.

These guides contain the same chemical modifications as chemistry #37 which was based on a 22 nt spacer, except that the modifications on the 5′ end where the spacer was shortened had to be adjusted. In mH29-29.1_37, the number of fluoro bases was reduced by 2, but the 4 PS bonds and 1 2′-O-methyl base at the 5′ remained the same as in the original #37 chemistry. In mH29-29.2_37, the number of fluoro bases was reduced by 2 and the 2′-O-methyl on the last base was retained, but the number of PS bonds at the 5′ end was increased from 4 to 5. The relative potency of these guides will be tested in cells in culture and in mice.

### Example 60—Design of a Guide RNA for MG29-1 with an 24 Nucleotide Stem-Loop Structure at the 5′ End to Improve Stability (Chemistry #50)

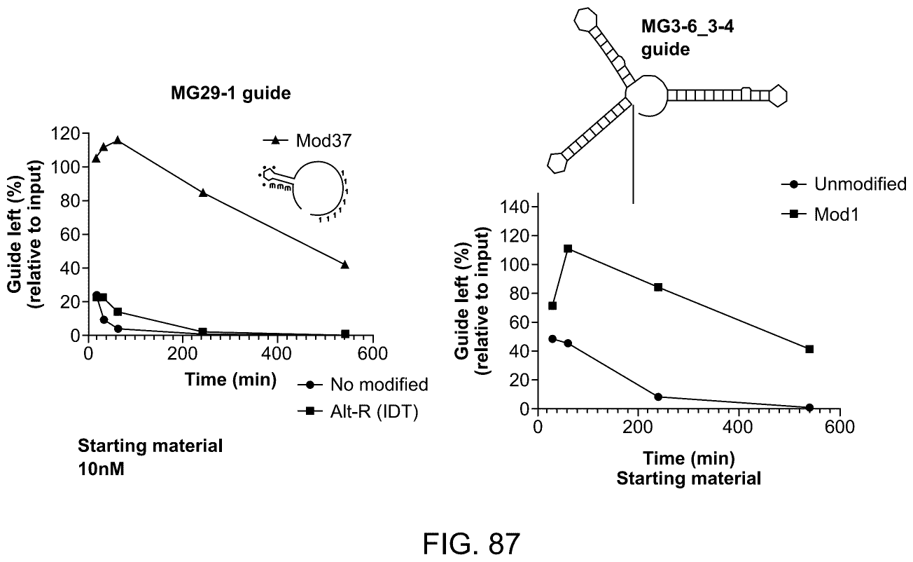

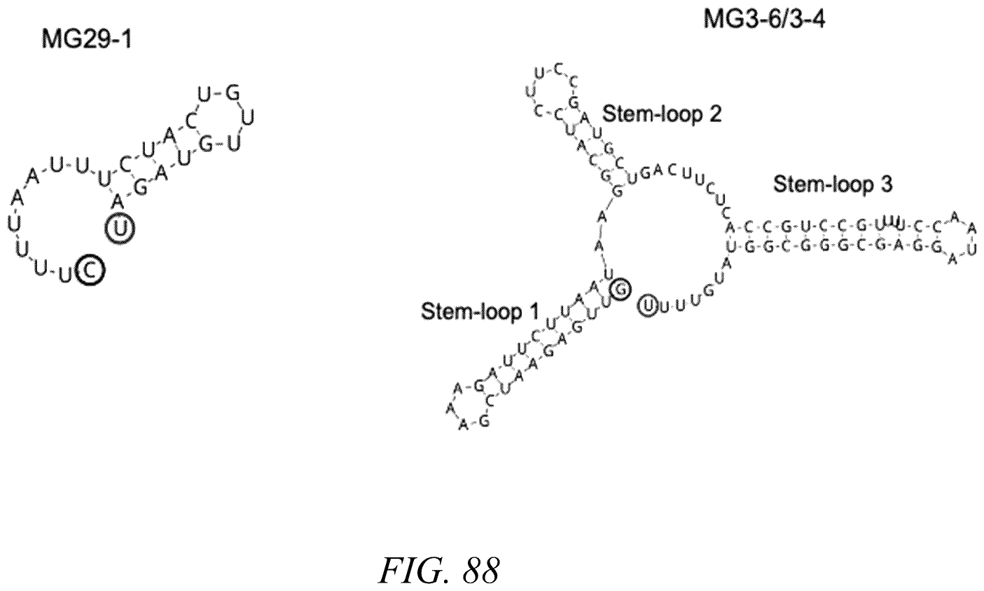

An in vitro stability assay for MG29-1 and MG3-6/3-4 guides demonstrated that MG29-1 guides can be less stable (FIG. 87). In this assay, the guide RNA was incubated in a crude extract from mammalian cells (Hepa1-6) that contains nucleases that can degrade RNA. An MG29-1 guide with chemical modifications on the 5′ and 3′ ends (Alt-R) was degraded in about 200 mins while about 50% of a MG3-6/3-4 guide with chemical modifications of the 5′ and 3′ ends (Mod 1) remained intact after 500 mins (FIG. 87).

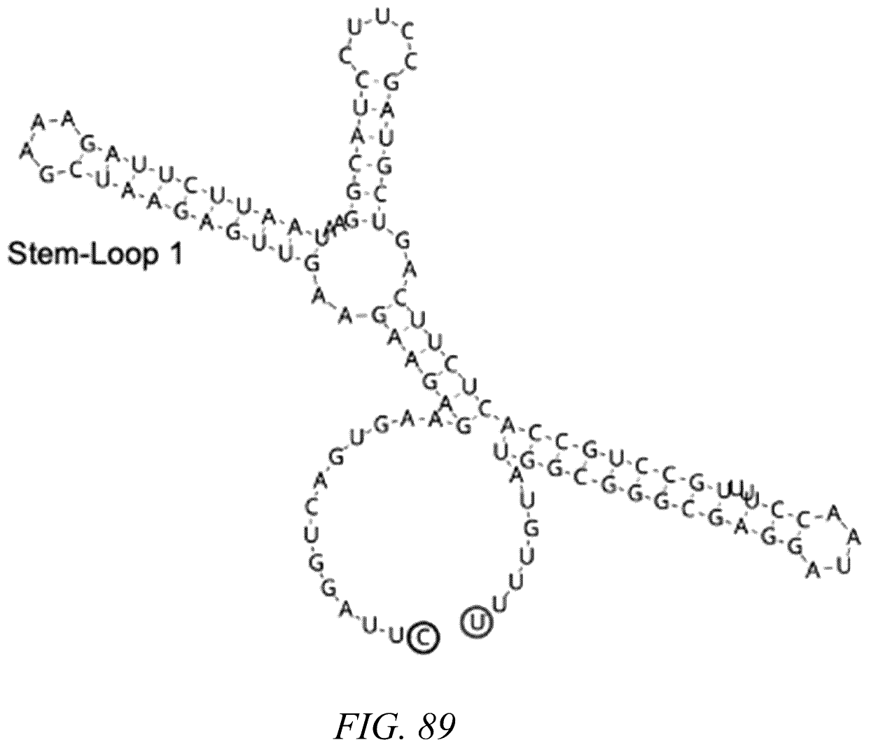

The structures of the MG29-1 and MG3-6/3-4 guide RNAs (FIG. 88) were predicted using the Geneious Prime Software (Turner 2004 algorithm: https://rna.urmc.rochester_edu/NNDB/index.html) and were noted to be significantly different. The MG29-1 guide is about one third of the length of the guide RNA for MG3-6/3-4. In addition, the MG29-1 guide contains minimal secondary structure comprising one stem of 5 nucleotides in length. In contrast, the guide RNA for MG3-6/3-4 contains 3 stem-loops with stem lengths of 10, 6, and 10 nucleotides (FIG. 88). The highly active MG3-6/3-4 guide containing a spacer targeting mouse albumin that was used to generate the data in FIG. 86 was also predicted to contain a stem of 10 nucleotides (Stem-loop 1 in FIG. 89) identical to the 10 nt stem-loop predicted for the backbone alone.

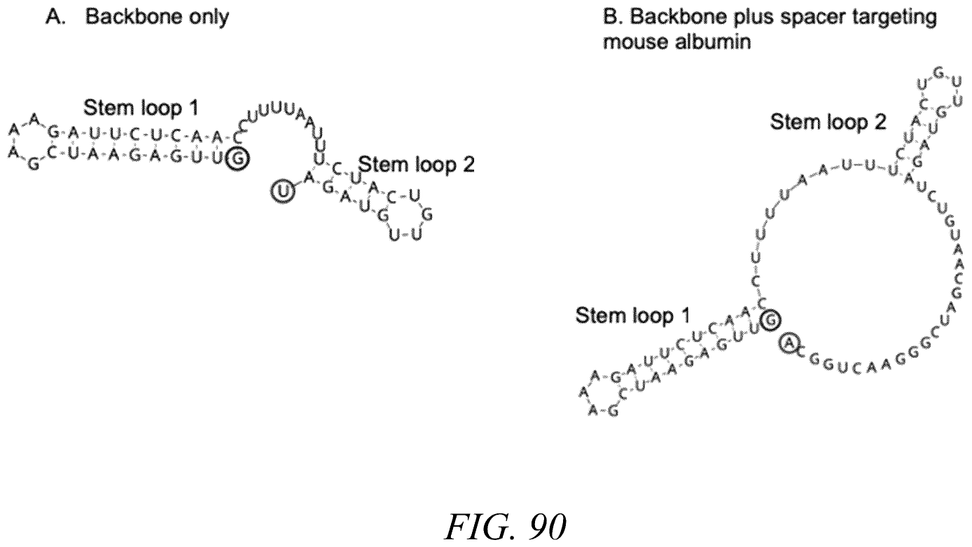

It was hypothesized that the minimal secondary structure of the MG29-1 guide made it less stable in a cellular milieu that is mimicked by the in vitro stability assay. Given the significantly greater in vitro stability of the MG3-6/3-4 guide RNA, a modified MG29-1 sgRNA backbone was designed in which the largest stem loop of MG3-6/3-4 (Stem-loop 1) was added at the 5′ end of the MG29-1 guide. The predicted structure of this modified MG29-1 guide is shown in FIG. 89. The chemical modifications designated chemistry #37 that had been previously demonstrated to significantly improve the stability and activity of the standard MG29-1 guide RNA were incorporated in the design of chemistry #50; specifically, the same phosphorothioate, 2′-O-methyl, and 2′-fluoro modifications present in the backbone and spacer of chemistry #37 were included in chemistry #50. To further stabilize this new design, the 3 nucleotides at the 5′ end were modified with phosphorothioate linkages and 2′-O-methyl bases. In addition, phosphorothioate linkages and 2′-O-methyl bases were included in the loop of the added stem loop (stem loop 1 in FIG. 90).

Additional variants of chemistry #50 were designed as shown in Table 32 below. The designs for chemistries #44, #50, #51, #52, #53, and #54 for any spacer sequence are shown in SEQ ID NOs: 5695-5701, in which N is any ribonucleotide base in the spacer.

TABLE 32
Summary of chemistry #50 and additional variants

| | | Spacer | Extra | Chemistry | |
|---|---|---|---|---|---|
| | SEQ | length | stem | on | |
| Guide name | ID | (nt) | loop | spacer/stem 2 | Additional changes |
| mAlb29-8-50 | 5689 | 22 | Yes | #37 | |
| mAlb29-8-50b | 5690 | 20 | Yes | #37 | |
| mAlb29-8-51b | 5691 | 20 | Yes | No 2′-fluoro in | |
| | | | | spacer | |
| mAlb29-8-52b | 5692 | 20 | Yes | Reduced 2′- | |
| | | | | fluoro in spacer | |
| mAlb29-8-53b | 5693 | 20 | Yes | #37 | PS and methyl on every |
| | | | | | base in stem loop 1 |
| mAlb29-8-54b | 5694 | 20 | Yes | No 2′-fluoro in | PS and methyl on every |
| | | | | spacer | base in stem loop 1 |

### Example 61—In Vitro Editing with MG29-1 Guide Chemistry #50 Containing a 24 Nucleotide Stem-Loop Structure at the 5′ End

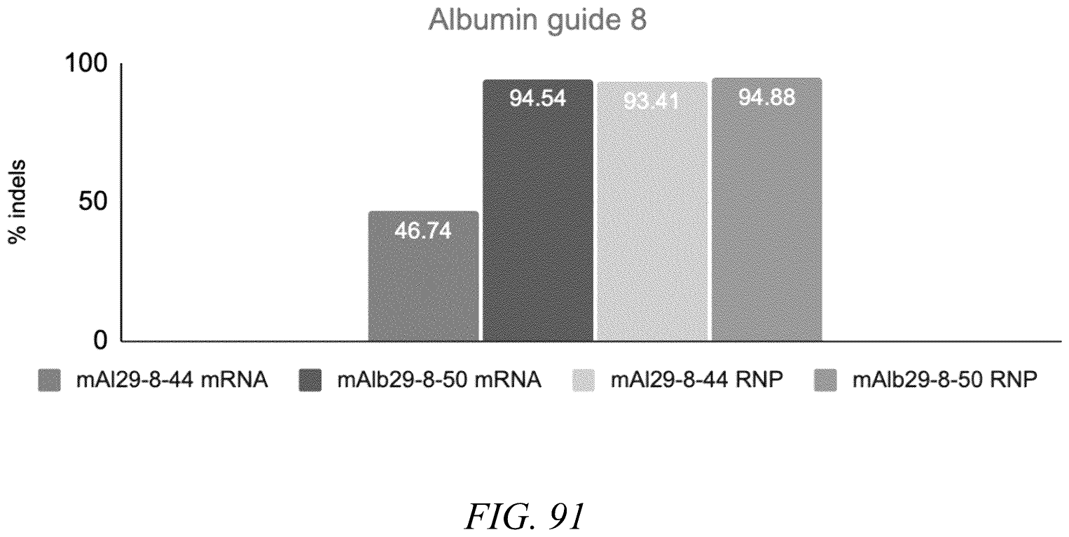

The mouse liver cell line Hepa1-6 (1×10⁵ cells/sample) was electroporated using an Amaxa nucleofection device and program: EH-100 with either pre-formed ribonucleoprotein complex (120 pmol MG29-1 protein mixed with 160 pmol guide RNA) or with a mixture of 500 ng MG29-1 miRNA and 210 pmol guide RNA. The guides tested were mA1b29-8-44 (spacer 8, chemistry 44) and mA1b29-8-50 (spacer 8, chemistry 50). Chemistry 44 comprises the MG29-1 backbone plus the 22 nt spacer and a specific set of chemical modifications of either the bases or the backbone that had been optimized for activity and stability. The sequence of mALb29-8-44 with chemical modifications is shown in SEQ ID NO: 5688. The sequence of mA1b29-8-50 is shown in SEQ ID NO: 5689. Both of these guides contain 22 nucleotide spacers. After electroporation, the cells were plated in 24 well plates and cultured for 3 days after which genomic DNA was purified from the cells using a commercial kit (Purelink, Invitrogen). The genomic DNA was analyzed for editing at the on target site (albumin intron 1) by next generation sequencing (NGS). The NGS data was analyzed by a custom Python script (IndelCalculator v11.3.1). As shown in FIG. 91, when the MG29-1 nuclease was delivered by mRNA transfection, chemistry 50 improved editing efficiency from 46% to 94%. When the MG29-1 nuclease was delivered as a protein that was pre-complexed to the guide, both chemistries exhibited editing of 94%. When the nuclease is delivered as a mRNA, the guide is ideally stable enough to survive inside the cell until the mRNA is translated into protein, after which the nuclease can complex with the guide and transit to the nucleus. Thus guide stability is likely more critical when the nuclease is delivered as mRNA. In contrast, the guide may be stabilized when complexed with the nuclease as a RNP prior to electroporation into the cells, in line with the observation that both chemistry 44 and 50 resulted in 94% editing by RNP electroporation (FIG. 91). These data suggest that chemistry 50 on the MG29-1 guide RNA containing an additional stem loop mediates improved editing in cells in culture.

### Example 62—In Vivo Gene Editing in the Liver of Mice with MG29-1 Guide Chemistry 50

To evaluate the impact of different guide chemistries on gene editing in the liver of mice, we delivered the MG29-1 mRNA and the guide RNA using a lipid nanoparticle (LNP). Messenger RNA encoding the MG29-1 nuclease was generated by in vitro transcription of a linearized plasmid template using T7 RNA polymerase and a mixture of ribonucleotides rATP, rCTP, and rGTP and N1-methyl pseudouridine in place of rUTP and CleanCAP (Trilink). The plasmid also encoded an approximately 100 nt polyA tail at the 3′ end of the coding sequence. The mRNA was purified on commercial spin columns, the concentration was determined by absorbance at 260 nM, and the purity was determined by Tape Station (Agilent). Three different guide RNAs that comprise the same spacer sequence but with different chemistries/backbones were evaluated in a single mouse study: mA1b29-8-44 (SEQ ID NO: 5688), mA1b29-8-50 (SEQ ID NO: 5689), and mA1b29-8-37 (SEQ ID NO: 5702). Guide mA1b29-12-44 (SEQ ID NO: 5703), which contains a different spacer (spacer 12) with chemistry 44, was also tested. The MG29-1 mRNA and the guide RNA were separately packaged inside lipid nanoparticles (LNP) using a process essentially as described by Kaufmann et al (PMID: 26469188, DOI:10.1021/acs.nanolett.5b02497, which is incorporated by reference herein in its entirety). Lipids were purchased from Avanti Polar Lipids or from Corden Pharma and dissolved in ethanol. The mRNA or sgRNA was prepared in water then diluted in 100 mM sodium acetate (pH 4.0) to make the RNA working stock. The four lipid components were combined in ethanol at specified ratios to make the lipid working stock. An example lipid mixture comprised cholesterol, DOPE, C12-200, and DMG-PEG-2000 at molar ratios of 47.5:16:35:1.5. The lipid working stock and the RNA working stock were combined in a microfluidics mixing device (Precision Nanosystems) at a flow rate of 12 mL/min and a ratio of 1 volume of lipid working stock to 3 volumes of RNA working stock. The mass ratio of C12-200 to RNA in the formulation was 10 to 1. The formulated LNP were diluted 1:1 with 1× PBS then dialyzed twice in 1× PBS for 1 hour each followed by concentration in Amicon spin concentrators. The resultant LNPs were formulated into 1× PBS buffer, filter sterilized through a 0.2 uM filter, and stored at 4° C. The concentration of the RNA inside and outside of the LNP was measured using the Ribogreen reagent (Thermo Fisher). The average diameter and polydispersity of the LNPs were measured in the resultant concentrated LNPs by dynamic light scattering using a NanoBrook 90Plus (Brookhaven Instruments). Representative LNPs ranged in size from 80 to 100 nanometers with a PDI<0.15 and an RNA encapsulation ratio of greater than 90%. LNP encapsulating a guide RNA and the MG29-1 mRNA were mixed at an RNA mass ratio of 1:1 then injected intravenously into wild type C57Bl/6 mice via the tail vein at a dose of 0.5 mg of RNA per kg in a total volume of 0.1 ml per mouse (N=5 mice per LNP). Mice were sacrificed at 4 days post dosing and the 3 lobes of the liver (left lateral, right lateral, medial) were collected, flash frozen, and stored at −80° C. The entire left lateral lobe of the liver was homogenized in Genomic Digestion Buffer (Purelink Genomic DNA Purification Kit, Thermo Fisher) using 0.4 mL of buffer per 100 mg of tissue weight in a Bead Mill. Genomic DNA was purified from an aliquot of the homogenate using the Purelink Genomic DNA Purification Kit (Thermo Fisher). The albumin intron 1 region was PCR amplified from 50 ng of the genomic DNA in a reaction containing 0.5 micro molar each of the primers mA1b90F (CTCCTCTTCGTCTCCGGC) and mA1b1073R (CTGCCACATTGCTCAGCAC) and 1× Pfusion Flash PCR Master Mix. The resulting 984 bp PCR product which spans the entire intron 1 of mouse albumin was purified using a column based purification kit (DNA Clean and Concentrator, Zymo Research) and sequenced using primers located within 150 to 350 bp of the predicted target site for each guide RNA. The PCR product generated using primers mA1b90F and mA1b1073R from a PBS buffer injected mouse was sequenced in parallel as a control. The Sanger sequencing chromatograms were analyzed using Inference of CRISPR Edits (ICE) that determines the frequency of INDELS as well as the INDEL profile (Hsiau et. al, Inference of CRISPR Edits from Sanger Trace Data. BioArxiv. 2018 https://www.biorxiv.org/content/early/2018/01/20/251082).

Without wishing to be bound by theory, it is understood that when a nuclease creates a double strand break (DSB) in DNA inside a living cell, the DSB is repaired by the cellular DNA repair machinery. In actively dividing cells, such as transformed mammalian cells in culture, and in the absence of a repair template, it is understood that this repair occurs by the NHEJ pathway. Without wishing to be bound by theory, it is understood that the NHEJ pathway is an error prone process that introduces insertions or deletions of bases at the site of the double strand break (Lieber, M. R, Annu Rey Biochem. 2010; 79: 181-211). These insertions and deletions are understood to be a hallmark of a double strand break that occurred and was subsequently repaired, and are thus widely used as a readout of the editing or cutting efficiency of the nuclease.

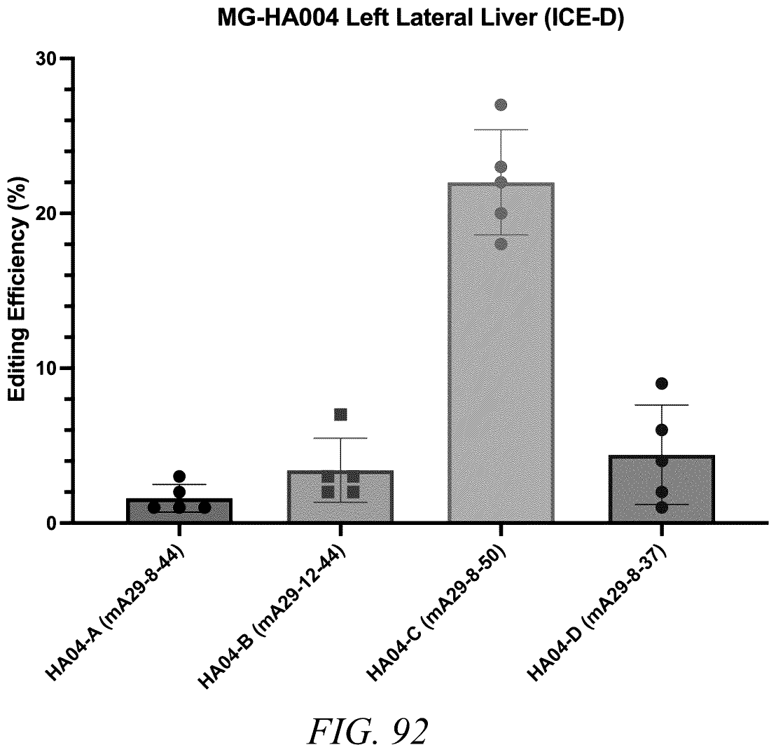

The editing efficiency in the liver of the mice is shown in FIG. 92. Comparing guides with the same spacer sequence (guide 8) but different chemistries or backbones, the guide with chemistry #44 (SEQ ID NO: 5688) was the least active (mean 1.6% editing) while the guide with chemistry #37 (SEQ ID NO: 5702) was more active (mean 4% editing). Chemistry #44 differs from chemistry #37 by the addition of 2 additional PS bonds in the spacer region (chemistry #44 has 6 PS bonds in the spacer region while chemistry #37 has 4 PS bonds in the spacer region). The guide with chemistry #50 (SEQ ID NO: 5689) was significantly more active with mean editing of 22%, approximately 5-fold higher than the guide with chemistry #37 and 10-fold higher than the guide with chemistry #44. When the same genomic DNA was analyzed for editing by next generation sequencing (NGS), the levels of editing with guide spacer 8 were determined to be 7%, 7%, and 42% for chemistries 44, 37, and 50, respectively. For guide spacer 12 with chemistry 44, the level of editing was 4% and 9% when measured by ICE and NGS, respectively confirming that chemistry 44 has similar activity to chemistry 37. These data demonstrate that chemistry #50, which contains a stem-loop from the MG3-6/3-4 guide added to the 5′ end of the normal MG29-1 guide backbone, exhibits significantly improved editing in the liver of mice after systemic delivery in an LNP. Thus, guide chemistry 50 provides improved in vivo potency of the MG29-1 nuclease.

### Example 63—Further Improvements to MG29-1 Guide Chemistry 50

Further improvements to the MG29-1 guide chemistry #50 are contemplated. In one potentially improved version, all of the nucleotides in the stem-loop 1 that was added to the 5′ end of the standard MG29-1 guide backbone are chemically modified with both 2′-O-methyl on the bases and phosphorothioate linkages as in chemistries 53 (SEQ ID NO: 5700) and 54 (SEQ ID NO: 5701). In one potentially improved version, all of the 2′-flouro bases in the spacer are changed to standard nucleotides as in chemistries 51 (SEQ ID NO: 5698) and 54 (SEQ ID NO: 5701). In another potentially improved version, the number of 2′-flouro bases in the spacer are reduced by 2-fold as in chemistry 52 (SEQ ID NO: 5699). The reduction in the number of 2′-fluoro bases may have impacts on guide specificity.

### Example 64—Design of a MG29-1 Single Guide RNA Comprising the Native Guide Array

An alternative approach to improving the stability, and thus the potency, of the MG29-1 single guide RNA is to design a native like CRISPR array for MG29-1, mimicking the documented process in which MG29-1 nuclease cleaves its own CRISPR array to generate a mature guide. The array was designated as mA1b29-g8-37-array (SEQ ID NO: 5712) and it comprises two copies of a 22 nt spacer targeting mouse albumin (spacer 8) embedded in the native CRISPR array for MG29-1.

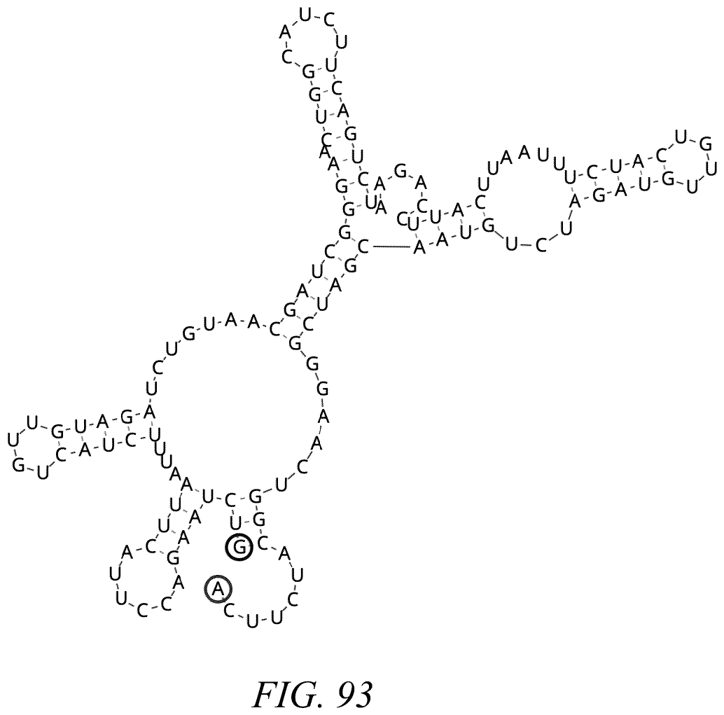

The designed array is 126 nt long and it comprises a repeat, followed by a spacer, followed by a repeat, followed by a spacer. The predicted secondary structure of mA1b29-g8-37-array is shown in FIG. 93 in which the 5′ end is circled in blue and the 3′ end is circled in red. This RNA is designed to be cleaved inside mammalian cells by an expressed MG29-1 nuclease to generate two functional sgRNAs. Chemical modifications were included in mA1b29-g8-37-array to promote stability. The modifications in the spacer and the MG29-1 backbone portions are based on those used in chemistry #37 but with additional modifications and some changes.

### Example 65—Guides for MG29-1 Nuclease with 20 nt Spacers Targeting Human Albumin Intron 1 with Chemistry #37

A guide screen against human albumin intron 1 using the MG29-1 nuclease and single guide RNA with 22 nt spacers identified 5 guides with high editing activity in human Hep3B cells. These guides were designated as spacer numbers 87, 78, 74, 83, and 84. Versions of these single guide RNA's with 20 nt spacers were designed incorporating the chemistry #37 chemical modifications and these were designated as hA29-87-37B (SEQ ID NO: 5713), hA29-78-37B (SEQ ID NO: 5714), hA29-74-37B (SEQ ID NO: 5715), hA29-83-37B (SEQ ID NO: 5716), and hA29-84-37B (SEQ ID NO: 5717).

### Example 66—Guides for MG29-1 Nuclease with 20 nt Spacers Targeting Human HAO1 with Chemistry #37

A guide screen against human HAO-1 (encoding glycolate oxidase) using the MG29-1 nuclease and single guide RNA with 22 nt spacers identified 4 guides with high editing activity in human Hep3B cells. These guides were designated as spacer numbers 4, 21, 23, and 41. Versions of these single guide RNA's with 20 nt spacers were designed incorporating the chemistry #37 chemical modifications and these were designated as hH29-4_37b (SEQ ID NO: 5718), hH29-21_37b (SEQ ID NO: 5719), hH29-23_37b (SEQ ID NO: 5720), and hH29-41_37b (SEQ ID NO: 5721).

### Example 67—Guides for MG29-1 Nuclease with 22 nt Spacers Targeting Human HAO1 with Chemistry #50

A guide screen against human HAO-1 (encoding glycolate oxidase) using the MG29-1 nuclease and single guide RNA with 22 nt spacers identified 4 guides with high editing activity in human Hep3B cells. These guides were designated as spacer numbers 4, 21, 23, and 41. Versions of these single guide RNA's with 22 nt spacers were designed incorporating the chemistry #50 chemical modifications and these were designated as hH29-4_50 (SEQ ID NO: 5722), hH29-21_50 (SEQ ID NO: 5723), hH29-23_50 (SEQ ID NO: 5724), and hH29-41_50 (SEQ ID NO: 5725).

### Example 68—Guides for MG29-1 with 20 nt Spacers Targeting Human HAO1 with Chemistry #50

A guide screen against human HAO-1 (encoding glycolate oxidase) using the MG29-1 nuclease and single guide RNA with 22 nt spacers identified 4 guides with high editing activity in human Hep3B cells. These guides were designated as spacer numbers 4, 21, 23, and 41. Versions of these single guide RNA's with 20 nt spacers were designed incorporating the chemistry #50 chemical modifications and these were designated as hH29-4_50b (SEQ ID NO: 5726), hH29-21_50b (SEQ ID NO: 5727), hH29-23_50b (SEQ ID NO: 5728), and hH29-41_50b (SEQ ID NO: 5729).

### Example 69—Guides for MG29-1 with 22 nt Spacers Targeting Mouse HAO1 with Chemistry #50

A guide screen against mouse HAO-1 (encoding glycolate oxidase) using the MG29-1 nuclease and single guide RNA with 22 nt spacers identified 3 guides with high editing activity in mouse Hepa1-6 cells. These guides were designated as spacer numbers 1, 15, and 29. Versions of these single guide RNA's with 22 nt spacers were designed incorporating the chemistry #50 chemical modifications and these were designated as mH29-1-50 (SEQ ID NO: 5730), mH29-15-50 (SEQ ID NO: 5731), and mH29-29-50 (SEQ ID NO: 5704).

### Example 70—Guides for MG29-1 with 20 nt Spacers Targeting Mouse HAO1 with Chemistry #50

A guide screen against mouse HAO-1 (encoding glycolate oxidase) using the MG29-1 nuclease and single guide RNA with 22 nt spacers identified 4 guides with high editing activity in mouse Hepa1-6 cells. These guides were designated as spacer numbers 1, 15, and 29. Versions of these single guide RNA's with 20 nt spacers were designed incorporating the chemistry #50 chemical modifications and these were designated as mH29-1-50b (SEQ ID NO: 5732), mH29-15-50b (SEQ ID NO: 5733), and mH29-29-50b (SEQ ID NO: 5705).

### Example 71—Comparison of the In Vivo Editing Efficiency of MG29-1 to spCas9

To compare the in vivo editing efficiency of the MG29-1 nuclease to that of spCas9, a dose response was performed in wild type C57B16 mice. Albumin intron 1 was selected as a genomic target locus for both spCas9 and MG29-1. An in silico search for spCas9 guide target sites in mouse intron 1 using the Chop-Chop algorithm (see e.g. Labun et al doi: 10.1093/nar/gkz365, which is incorporated by reference in its entirety herein) identified a total of 39 potential guides, which were ranked according to their efficiency score and off-target prediction. In addition, guide target sites located within 50 bp of exon 1 or exon 2 were excluded. The top 3 guides from this ranking were designated mA1bR1 (SEQ ID NO: 5734), mA1bR2 (SEQ ID NO: 5735), and mA1bR3 (SEQ ID NO: 5736), and were chemically synthesized with chemical modifications at both the 5′ and 3′ ends comprising methylated bases (represented by the nomenclature mA, mC, mG, and mU) and phosphorothioate backbone linkages (represented by the nomenclature A*, C*, G*, and U*). The editing efficiencies of these 3 guides were evaluated in the mouse liver cell line Hepa1-6 by nucleofection of ribonucleoprotein complexes formed by mixing the guide RNA and commercially sourced spCas9 protein (purchased from Integrated DNA technologies) at a molar ratio of 1:2.5 (protein to guide RNA). 20 moles of spCas9 protein was mixed with 50 moles of guide RNA and subsequently nucleofected into 2×10⁵ Hepa1-6 cells using an Amaxa electroporation device with program setting EH100. The nucleofected cells were each transferred to a well of a 48 well plate in fresh growth media and cultured for 48 h in a 5% CO₂/37° C. humidified incubator. Genomic DNA was purified from the cells using the Purelink kit (Invitrogen, ThermoFisher) and analyzed for editing at the target site in albumin intron 1 by PCR amplification of the target locus using primers mA1b90F and mA1b1073R (SEQ ID NOs: 5737 and 5738) and a high fidelity PCR enzyme mix. The PCR product was subjected to Sanger sequencing using primers mA1b282F or mA1b460F. The Sanger sequencing chromatograms were analyzed for insertions and deletions (“indels”) at the predicted target site for each guide by Tracking of Indels by DEcomposition (TIDE) as described by Brinkman et al (Nucleic Acids Res. 2014 Dec. 16; 42 (22), doi: 10.1093/nar/gku936, which is incorporated by reference in its entirety herein). The presence of indels at the target site is the consequence of the generation of double strand breaks in the DNA, which are then repaired by the error prone cellular repair machinery which introduces insertions and deletions. The results of the TIDE analysis are shown in Table 33. All three guides generated indel frequencies of greater than 90%, demonstrating that all three guides are highly active.

TABLE 33
INDEL frequencies in Hepa1-6 cells nucleofected with guide RNA for
spCas9 targeting mouse albumin intron 1 and spCas9 protein as a RNP

| Sample | | | |
|---|---|---|---|
| ID | Guide | INDEL % | R2 |
| 1 | mAlbR1 | 92 | 0.95 |
| 2 | mAlbR2 | 91 | 0.91 |
| 3 | mAlbR3 | 96 | 0.96 |

Guide mALbR2 was synthesized with extensive chemical modifications as described previously (Yin et al. doi:10.1038/nbt.4005, and WO 2019/079527 A1, each of which are incorporated by reference in their entirety herein). The chemical modifications include modifications of the 3 bases at the 5′ end and 3 bases at the 3′ end with 2′-O-methyl bases and phosphorothioate linkages between the 3 bases at the 5′ end and the 3 bases at the 3′ end. In addition, 33 of the internal bases are modified with 2′-O-methyl (SEQ ID NO: 5741). These chemical modifications of the guide RNA for spCas9 were reported to enable efficient editing in vivo in mouse liver after delivery of the mRNA for spCas9 and the guide RNA in a lipid nanoparticle (see e.g. WO 2019/079527 A1, which is incorporated by reference in its entirety herein).

A guide screen for guides that target the MG29-1 nuclease to mouse albumin intron 1 and promote cleavage and indel formation was performed. The two guides with the highest editing activity in Hepa1-6 cells when the nuclease was delivered as a mRNA were mALb29-8 and mA1b29-12. Guide mALb29-8 was selected for comparison to spCas9 guide mA1bR2 in vivo in mice. Chemical and structural modifications to the guide RNA for MG29-1 were optimized by evaluating the impact of different chemical modifications including 2′O-methyl and 2′-fluoro modified bases, phosphorothioate linkages, as well as an additional stem loop upon the stability and editing activity of the guide.

Experiments on guide chemistry optimization indicated that guide chemistry #50 was the most active guide chemistry among those tested. When delivered in vivo to mice using a LNP encapsulating MG29-1 mRNA and the same guide RNA sequence targeting mouse albumin intron 1, but with two different guide chemistries (#37 and #50), chemistry #50 was about 4-fold more potent than chemistry #37 at a dose of 0.5 mg/kg. Therefore, MG29-1 guide chemistry #50 was selected to test in vivo in comparison to spCas9 with its cognate guide mALbR2 (SEQ ID NO: 5741).

Messenger RNA encoding the MG29-1 nuclease or the spCas9 nuclease was generated by in vitro transcription of a linearized plasmid template using T7 RNA polymerase and a mixture of ribonucleotides rATP, rCTP, and rGTP, N1-methyl pseudouridine, and the CleanCAP capping reagent (Trilink Biotechnologies). The SV40-derived nuclear localization sequence (PKKKRKVGGGGS) followed by a short linker was included at the N terminus of the coding sequence of both spCas9 and MG29-1. The nuclear localization signal from nucleoplasmin preceded by a short linker (SGGKRPAATKKAGQAKKKK) was added to the C-terminus of the coding sequence for both spCas9 and MG29-1. Thus, the same nuclear localization signals were used for both MG29-1 and spCas9. The plasmids also encoded an approximately 100 nt polyA tail at the 3′ end of both spCas9 and MG29-1 coding sequences, which generates a polyA tail in the mRNA. The coding sequences for both spCas9 and MG29-1 were codon optimized using the same algorithm (see e.g. Raab et al, DOI 10.1007/s11693-010-9062-3, which is incorporated by reference in its entirety herein). The DNA sequence encoding the spCas9 mRNA is in SEQ ID NO: 5742 and the amino acid sequence encoded by the spCas9 mRNA is in SEQ ID NO: 5743. The mRNA was purified on commercial spin columns, the concentration was determined by absorbance at 260 nM, and the purity was determined by Tape Station (Agilent); the purity was found to be equivalent for both spCas9 mRNA and MG29-1 mRNA. For in vivo delivery to mice, the spCas9 mRNA/mA1bR2 guide or the MG29-1 mRNA/mA1b29-8-50 guide were packaged inside lipid nanoparticles (LNP) using a process essentially as described by Kaufmann et al. (PMID: 26469188, DOI:10.1021/acs.nanolett.5b02497, which is incorporated by reference herein). The guide RNA and the mRNA were separately packaged for both spCas9 and for MG29-1. Lipids (purchased from Avanti Polar Lipids or from Corden Pharma) were dissolved in ethanol. The mRNA or guide RNA was prepared in water, then diluted in 100 mM sodium acetate (pH 4.0) to make the RNA working stock. The four lipid components were combined in ethanol at the specified ratios to make the lipid working stock. An example lipid mixture comprised cholesterol, a neutral lipid such as 1,2-dioleoyl-sn-glycero-3-phosphoethanolamine (DOPE), a cationic lipid such as 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), and a PEG-linked lipid such as 1,2-dimyristoyl-rac-glycero-3-methoxypolyethylene glycol-2000 (DMG-PEG-2000) at molar ratios of 47.5:16:35:1.5. The lipid working stock and the RNA working stock were combined in a microfluidics mixing device (Precision Nanosystems) at a flow rate of 12 mL/min and a ratio of 1 volume of lipid working stock to 3 volumes of RNA working stock. The mass ratio of C12-200 to RNA in the formulation was 10 to 1. The formulated LNP were diluted 1:1 with 1× PBS then dialyzed twice in 1× PBS for 1 hour each, followed by concentration in Amicon spin concentrators. The resultant LNP were formulated into 1× PBS buffer, filter sterilized through a 0.2 μM filter and stored at 4° C. The concentration of the RNA inside and outside of the LNP was measured using the Ribogreen reagent (Thermo Fisher). The average diameter and polydispersity of the LNP were measured in the resultant concentrated LNP by dynamic light scattering using a NanoBrook 90Plus (Brookhaven Instruments). Representative LNP ranged in size from 80 to 100 nanometers with a PDI<0.15 and an RNA encapsulation ratio of greater than 90%. The average diameter, polydispersity, and RNA encapsulation efficiency is shown below in Table 34.

TABLE 34
Summary of LNP characteristics

| | | Average | | Percent |
|---|---|---|---|---|
| | | Diameter | | encapsulation of |
| LNP | RNA | (nm) | Polydispersity | RNA |
| LNP-A | MG29-1 mRNA | 57 | 0.082 | 94.9 |
| LNP-B | mAlb29-8-50 guide | 47 | 0.082 | 93.7 |
| | (SEQ ID NO: | | | |
| | 5744) | | | |
| LNP-C | Cas9 mRNA | 60 | 0.114 | 94.5 |
| LNP-D | mAlbR2 guide | 50 | 0.059 | 94.5 |
| | (SEQ ID NO: | | | |
| | 5741) | | | |

LNP encapsulating the guide RNA mA1b29-8-50 and the MG29-1 mRNA were mixed at an RNA mass ratio of 1:1. LNP encapsulating the guide RNA mA1bR1 and the spCas9 mRNA were mixed at an RNA mass ratio of 1:1. Both LNP mixtures were injected intravenously into wild type C57BV/6 mice via the tail vein with total RNA doses of 1 mg/kg, 0.5 mg/kg, or 0.25 mg/kg of RNA in a total volume of 0.1 mL per mouse (N=5 mice per LNP dose). Mice were sacrificed at 5 days post-dosing and the whole liver was flash frozen and stored at −80° C. The entire left lateral lobe of the liver was homogenized in Genomic Digestion Buffer (Purelink Genomic DNA Purification Kit, Thermo Fisher) using 0.4 mL of buffer per 100 mg of tissue weight in a Bead Mill. Genomic DNA was purified from an aliquot of the homogenate using the Purelink Genomic DNA Purification Kit (Thermo Fisher). The albumin intron 1 region was PCR amplified from 50 ng of the genomic DNA in a reaction containing 0.5 micromolar each of the primers mA1b90F (SEQ ID NO: 5737, CTCCTCTTCGTCTCCGGC) and mA1b1073R (SEQ ID NO: 5738, CTGCCACATTGCTCAGCAC) and 1× Pfusion Flash high fidelity PCR Master Mix. The resulting 984 bp PCR product, which spans the entire intron 1 of mouse albumin, was purified using a column-based purification kit (DNA Clean and Concentrator, Zymo Research). The PCR product was sequenced by next generation sequencing (NGS), analyzing for creation of indels in the target sequence, which were used as indicators of creation of double-strand breaks by the Cas enzymes and engagement of the NHEJ pathway. This detection method is based in the concept that when a nuclease creates a double strand break (DSB) in DNA inside a living cell, the DSB is believed to be repaired by the cellular DNA repair machinery which, in the absence of a repair template, occurs by the NHEJ pathway. As the NHEJ pathway is known to be an error prone process that introduces insertions or deletions of bases at the site of the double strand break (Lieber, M. R, Annu Rev Biochem 2010: 79: 181-211), these insertions and deletions (indels) are therefore used as a hallmark of a double strand break that occurred and was subsequently repaired, and thus as a readout of the editing or cutting efficiency of the nuclease.

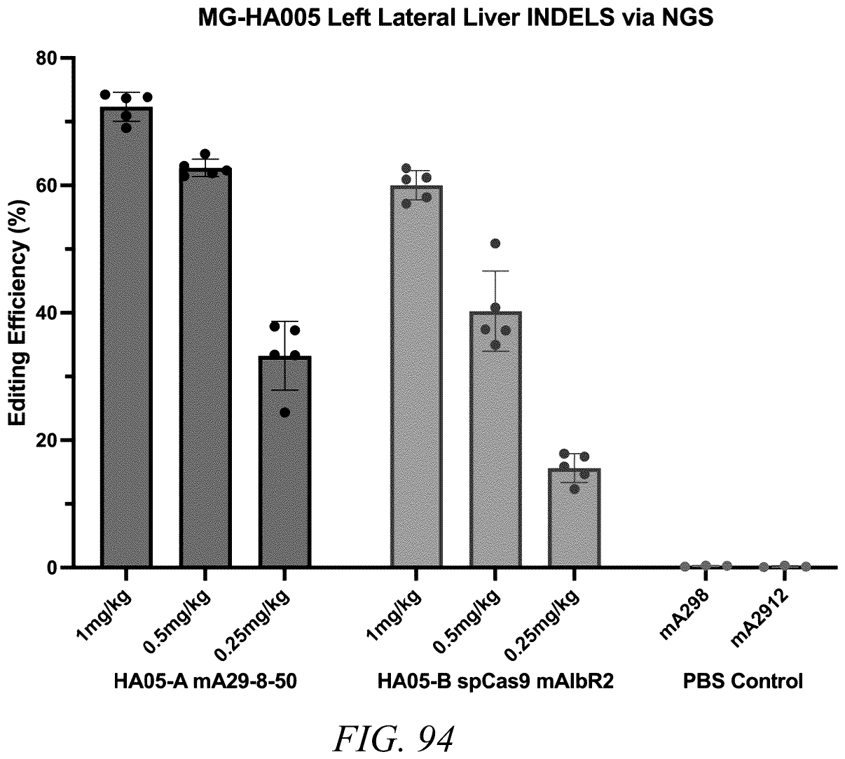

The sequencing reads were analyzed with a custom Python script (IndelCalculator v1.3.1) that aligns each sequence read to the wild-type target sequence (in this case Albumin intron 1) and calculates the number of reads that contain at least one indel irrespective of the indel size within a window that spans 10 base pairs either side of the predicted on-target cut site for the nuclease. The editing efficiency (indel frequency) in each of the 5 mice in each group, as well as the mean and standard deviation for the group, are summarized in FIG. 94. No editing was detected in the control mice injected with PBS buffer. Both spCas9 mRNA/mA1bR2 LNP and the MG29-1 mRNA/mA1b29-8-50 LNP resulted in dose-dependent editing. At all 3 doses, the editing efficiency was higher for the MG29-1 mRNA/mA1b29-8-50 LNP than for the spCas9 mRNA/mA1bR2 LNP. The mean editing efficiencies at the 3 doses are summarized in Table 35. At a dose of 1 mg/kg (0.5 mg/kg mRNA and 0.5 mg/kg guide RNA), MG29-1 was slightly more potent than spCas9, resulting in about 15% more indels. At a dose of 0.5 mg/kg (0.25 mg/kg mRNA and 0.25 mg/kg guide RNA), MG29-1 resulted in about 50% more indels. At a dose of 0.25 mg/kg (0.125 mg/kg mRNA and 0.125 mg/kg guide RNA), MG29-1 resulted in 100% more indels. These data demonstrate that using the same LNP for delivery and mRNA produced using an identical process, the MG29-1 nuclease combined with an appropriately optimized guide RNA is more potent than the spCas9 nuclease and an appropriately modified guide RNA. The superior in vivo editing efficiency of MG29-1 was especially evident at the lowest dose tested, where MG29-1 was 2-fold more potent than spCas9 at the same dose. These results suggest that the MG29-1 nuclease and an appropriately modified guide RNA exemplified by chemistry #50 may have an advantage for in vivo gene editing using LNP delivery.

TABLE 35
Mean editing efficiency in the whole liver of mice at 5 days after intravenous
injection of LNP encapsulating either MG29-1 mRNA and guide mAlb29-8-50 (mA29-8-50)
or spCas9 mRNA and guide mAlbR2 at three doses, or PBS buffer (Control).

| | | | Mean editing | Standard |
|---|---|---|---|---|
| mRNA | Guide RNA | Dose (mg/kg) | (%) | deviation |
| MG29-1 | mAlb29-8-50 | 1 | 72.3 | 2.3 |
| MG29-1 | mAlb29-8-50 | 0.5 | 62.7 | 1.4 |
| MG29-1 | mAlb29-8-50 | 0.25 | 33.2 | 5.4 |
| spCas9 | mAlbR2 | 1 | 60.0 | 2.3 |
| spCas9 | mAlbR2 | 0.5 | 40.3 | 6.3 |
| spCas9 | mAlbR2 | 0.25 | 15.6 | 2.3 |
| PBS control | | — | 0.18 | 0.1 |

### Example 72—Identification of Active Single Guide RNA for MG29-1 that Target the Exonic Regions of Human HAO-1 by mRNA-Based Transfection in Hep3B Cells

Sequence-specific nucleases can be used to disrupt the coding sequence of a gene of interest, thereby creating a functional knockout of the protein encoded by that gene. This can be of therapeutic use when the knockdown of the protein has a beneficial effect in a particular human disease. One way to disrupt the coding sequence of a gene is to make a double strand break within the exonic regions of the gene using a sequence-specific nuclease. The double strand break is repaired via error-prone repair pathways, primarily non-homologous end joining (NHEJ) to generate insertions or deletions which can result in either frameshift mutations or changes to the amino acid sequence which disrupt the function of the protein.

To identify guide RNAs for MG29-1 that efficiently create double strand breaks at exonic regions of the gene encoding human glycolate oxidase (GO), single guide RNAs (sgRNAs) with a spacer length of 22 nt targeted to exons 1 to 4 of the human hydroxyacid oxidase (HAO-1) gene (GenBank RefSeq accession number NG 046733) were identified using the guide finding algorithm in the Geneious Prime nucleic acid analysis software (https://www.geneious.com/prime/).

The first four exons of the human HAO-1 gene encode amino acids comprising approximately the N-terminal 50% of the HAO-1 coding sequence. The first 4 exons were chosen because indels created towards the N-terminus of the coding sequence of a gene are more likely to create a frameshift or missense mutation that disrupts the activity of the protein. Using the more restrictive PAM for MG29-1 of TTTN, a total of 42 potential sgRNAs were identified within human HAO-1 exons 1 through 4. Guides that spanned the intron/exon boundaries were included because such guides may create INDELS that interfere with splicing. To create the full sgRNAs, the backbone sequence (UAAUUUCUACUGUUGUAGAU) was added to the 3′ end of the spacer sequence. The sgRNAs were chemically synthesized incorporating chemically modified bases documented to improve the performance of sgRNAs for the type V nuclease cpf1 (“A1tR1/A1tR2” chemistry, commercially available at Integrated DNA Technologies). The spacer sequences of these guides are shown in Table 36.

TABLE 36
Sequences of 42 single guide RNA targeting human HAO-1 for
testing in human Hep3B cells

| Guide | SEQ | | | Spacer | |
|---|---|---|---|---|---|
| Name | ID | Exon | PAM | Sequence (as DNA) | sgRNA Sequence |
| hH29-1 | 4184 | 1 | TTTA | GCATGTTGTTCATAATC | UAAUUUCUACUGUUGUA |
| | | | | ATTGA | GAUGCAUGUUGUUCAUA |
| | | | | | AUCAUUGA |
| hH29-2 | 4185 | 1 | TTTG | GAAGTACTGATTTAGCA | UAAUUUCUACUGUUGUA |
| | | | | TGTTG | GAUGAAGUACUGAUUUA |
| | | | | | GCAUGUUG |
| hH29-3 | 4186 | 1 | TTTG | TATCAATGATTATGAAC | UAAUUUCUACUGUUGUA |
| | | | | AACAT | GAUUAUCAAUGAUUAUG |
| | | | | | AACAACAU |
| hH29-4 | 4187 | 1 | TTTG | CCCCAGACCTGTAATAG | UAAUUUCUACUGUUGUA |
| | | | | TCATA | GAUCCCCAGACCUGUAAU |
| | | | | | AGUCAUA |
| hH29-5 | 4188 | 1 | TTTC | TTCATCATTTGCCCCAGA | UAAUUUCUACUGUUGUA |
| | | | | CCTG | GAUUUCAUCAUUUGCCCC |
| | | | | | AGACCUG |
| hH29-6 | 4189 | 1 | TTTC | TTACCTGGAAAATGCTG | UAAUUUCUACUGUUGUA |
| | | | | CAATA | GAUUUACCUGGAAAAUG |
| | | | | | CUGCAAUA |
| hH29-7 | 4190 | 1 | TTTT | CTTACCTGGAAAATGCT | UAAUUUCUACUGUUGUA |
| | | | | GCAAT | GAUCUUACCUGGAAAAU |
| | | | | | GCUGCAAU |
| hH29-8 | 4191 | 1 | TTTG | GCTGATAATATTGCAGC | UAAUUUCUACUGUUGUA |
| | | | | ATTTT | GAUGCUGAUAAUAUUGC |
| | | | | | AGCAUUUU |
| hH29-9 | 4192 | 1 | TTTA | AAAAATAAATTTTCTTA | UAAUUUCUACUGUUGUA |
| | | | | CCTGG | GAUAAAAAUAAAUUUUC |
| | | | | | UUACCUGG |
| hH29-10 | 4193 | 1 | TTTT | AAAAAATAAATTTTCTT | UAAUUUCUACUGUUGUA |
| | | | | ACCTG | GAUAAAAAAUAAAUUUU |
| | | | | | CUUACCUG |
| hH29-11 | 4194 | 2 | TTTT | ATTTTATTTTTTAATTCT | UAAUUUCUACUGUUGUA |
| | | | | AGAT | GAUAUUUUAUUUUUUAA |
| | | | | | UUCUAGAU |
| hH29-12\| | 4195 | 2 | TTTA | TTTTATTTTTTAATTCTA | UAAUUUCUACUGUUGUA |
| | | | | GATG | GAUUUUUAUUUUUUAAU |
| | | | | | UCUAGAUG |
| hH29-13\| | 4196 | 2 | TTTT | ATTTTTTAATTCTAGATG | UAAUUUCUACUGUUGUA |
| | | | | GAAG | GAUAUUUUUUAAUUCUA |
| | | | | | GAUGGAAG |
| hH29-14 | 4197 | 2 | TTTA | TTTTTTAATTCTAGATGG | UAAUUUCUACUGUUGUA |
| | | | | AAGC | GAUUUUUUUAAUUCUAG |
| | | | | | AUGGAAGC |
| hH29-15 | 4198 | 2 | TTTT | TTAATTCTAGATGGAAG | UAAUUUCUACUGUUGUA |
| | | | | CTGTA | GAUUUAAUUCUAGAUGG |
| | | | | | AAGCUGUA |
| hH29-16\| | 4199 | 2 | TTTT | TAATTCTAGATGGAAGC | UAAUUUCUACUGUUGUA |
| | | | | TGTAT | GAUUAAUUCUAGAUGGA |
| | | | | | AGCUGUAU |
| hH29-17 | 4200 | 2 | TTTT | AATTCTAGATGGAAGCT | UAAUUUCUACUGUUGUA |
| | | | | GTATC | GAUAAUUCUAGAUGGAA |
| | | | | | GCUGUAUC |
| hH29-18 | 4201 | 2 | TTTA | ATTCTAGATGGAAGCTG | UAAUUUCUACUGUUGUA |
| | | | | TATCC | GAUAUUCUAGAUGGAAG |
| | | | | | CUGUAUCC |
| hH29-19 | 4202 | 2 | TTTC | AGCAACATTCCGGAGCA | UAAUUUCUACUGUUGUA |
| | | | | TCCTT | GAUAGCAACAUUCCGGAG |
| | | | | | CAUCCUU |
| hH29-20 | 4203 | 2 | TTTT | AGGACAGAGGGTCAGCA | UAAUUUCUACUGUUGUA |
| | | | | TGCCA | GAUAGGACAGAGGGUCA |
| | | | | | GCAUGCCA |
| hH29-21 | 4204 | 2 | TTTA | GGACAGAGGGTCAGCAT | UAAUUUCUACUGUUGUA |
| | | | | GCCAA | GAUGGACAGAGGGUCAG |
| | | | | | CAUGCCAA |
| hH29-22 | 4205 | 3 | TTTC | TTTCTCAGCCTGTCAGTC | UAAUUUCUACUGUUGUA |
| | | | | CCTG | GAUUUUCUCAGCCUGUCA |
| | | | | | GUCCCUG |
| hH29-23 | 4206 | 3 | TTTC | TCAGCCTGTCAGTCCCT | UAAUUUCUACUGUUGUA |
| | | | | GGGAA | GAUUCAGCCUGUCAGUCC |
| | | | | | CUGGGAA |
| hH29-24 | 4207 | 3 | TTTG | TGACAGTGGACACACCT | UAAUUUCUACUGUUGUA |
| | | | | TACCT | GAUUGACAGUGGACACAC |
| | | | | | CUUACCU |
| hH29-25 | 4208 | 3 | TTTG | AATCTGTTACGCACATC | UAAUUUCUACUGUUGUA |
| | | | | ATCCA | GAUAAUCUGUUACGCACA |
| | | | | | UCAUCCA |
| hH29-26 | 4209 | 4 | TTTT | ATGCATTTCTTATTTTAG | UAAUUUCUACUGUUGUA |
| | | | | GATG | GAUAUGCAUUUCUUAUU |
| | | | | | UUAGGAUG |
| hH29-27 | 4210 | 4 | TTTA | TGCATTTCTTATTTTAGG | UAAUUUCUACUGUUGUA |
| | | | | ATGA | GAUUGCAUUUCUUAUUU |
| | | | | | UAGGAUGA |
| hH29-28 | 4211 | 4 | TTTC | TTATTTTAGGATGAAAA | UAAUUUCUACUGUUGUA |
| | | | | ATTTT | GAUUUAUUUUAGGAUGA |
| | | | | | AAAAUUUU |
| hH29-29 | 4212 | 4 | TTTT | AGGATGAAAAATTTTGA | UAAUUUCUACUGUUGUA |
| | | | | AACCA | GAUAGGAUGAAAAAUUU |
| | | | | | UGAAACCA |
| hH29-30 | 4213 | 4 | TTTA | GGATGAAAAATTTTGAA | UAAUUUCUACUGUUGUA |
| | | | | ACCAG | GAUGGAUGAAAAAUUUU |
| | | | | | GAAACCAG |
| hH29-31 | 4214 | 4 | TTTC | CTCAGGAGAAAATGATA | UAAUUUCUACUGUUGUA |
| | | | | AAGTA | GAUCUCAGGAGAAAAUG |
| | | | | | AUAAAGUA |
| hH29-32 | 4215 | 4 | TTTT | CCTCAGGAGAAAATGAT | UAAUUUCUACUGUUGUA |
| | | | | AAAGT | GAUCCUCAGGAGAAAAU |
| | | | | | GAUAAAGU |
| hH29-33 | 4216 | 4 | TTTT | GAAACCAGTACTTTATC | UAAUUUCUACUGUUGUA |
| | | | | ATTTT | GAUGAAACCAGUACUUU |
| | | | | | AUCAUUUU |
| hH29-34 | 4217 | 4 | TTTG | AAACCAGTACTTTATCA | UAAUUUCUACUGUUGUA |
| | | | | TTTTC | GAUAAACCAGUACUUUA |
| | | | | | UCAUUUUC |
| hH29-35 | 4218 | 4 | TTTA | TCATTTTCTCCTGAGGAA | UAAUUUCUACUGUUGUA |
| | | | | AATT | GAUUCAUUUUCUCCUGAG |
| | | | | | GAAAAUU |
| hH29-36 | 4219 | 4 | TTTT | CTCCTGAGGAAAATTTT | UAAUUUCUACUGUUGUA |
| | | | | GGAGA | GAUCUCCUGAGGAAAAU |
| | | | | | UUUGGAGA |
| hH29-37 | 4220 | 4 | TTTC | TCCTGAGGAAAATTTTG | UAAUUUCUACUGUUGUA |
| | | | | GAGAC | GAUUCCUGAGGAAAAUU |
| | | | | | UUGGAGAC |
| hH29-38 | 4221 | 4 | TTTA | GCCACATATGCAGCAAG | UAAUUUCUACUGUUGUA |
| | | | | TCCAC | GAUGCCACAUAUGCAGCA |
| | | | | | AGUCCAC |
| hH29-39 | 4222 | 4 | TTTT | GGAGACGACAGTGGACT | UAAUUUCUACUGUUGUA |
| | | | | TGCTG | GAUGGAGACGACAGUGG |
| | | | | | ACUUGCUG |
| hH29-40 | 4223 | 4 | TTTG | GAGACGACAGTGGACTT | UAAUUUCUACUGUUGUA |
| | | | | GCTGC | GAUGAGACGACAGUGGA |
| | | | | | CUUGCUGC |
| hH29-41 | 4224 | 4 | TTTG | ATATCTTCCCAGCTGAT | UAAUUUCUACUGUUGUA |
| | | | | AGATG | GAUAUAUCUUCCCAGCUG |
| | | | | | AUAGAUG |
| hH29-42 | 4225 | 4 | TTTG | CAACAATTGGCAATGAT | UAAUUUCUACUGUUGUA |
| | | | | GTCAG | GAUCAACAAUUGGCAAU |
| | | | | | GAUGUCAG |

Hep3B cells (ATCC catalog number HB-8064), a transformed human liver cell line (derived from a hepatocellular carcinoma), were cultured under standard conditions in growth media (EMEM media with 10% FBS) in a 5% CO₂ incubator and transfected with a mixture of mRNA encoding MG29-1 and each of the single guide RNAs. The mRNA encoding MG29-1 was generated by T7 polymerase in vitro transcription of a plasmid in which the coding sequence of MG29-1 had been cloned. The MG29-1 coding sequence was codon optimized using human codon usage tables and flanked by nuclear localization signals derived from SV40 (at the N-terminus) and from Nucleoplasmin (at the C-terminus). In addition, a 5′ untranslated region (5′ UTR) was included at the 5′ end of the coding sequence to improve translation. A 3′ UTR followed by an approximately 90 to 110 nucleotide polyA tract was included in the mRNA (encoded in the plasmid) at the 3′ end of the coding sequence to improve mRNA stability in vivo. The DNA sequence that encodes the MG29-1 mRNA without the polyA tail is shown in SEQ ID 5830. The in vitro transcription reaction included the Clean Cap® capping reagent (Trilink BioTechnologies), the resulting RNA was purified using the MEGAClear™ Transcription Clean-Up kit (Invitrogen), and purity was evaluated using the TapeStation (Agilent) and found to be composed of >90% full length RNA.

When co-transfection of mRNA and guide with a lipid transfection reagent such as MessengerMAX is used, the mixture of the two RNA molecules forms a complex with the positively charged lipid, the complex enters the cells via endocytosis, and eventually some of the RNA reaches the cytoplasm. In the cytoplasm, the mRNA is translated into protein. In the case of an RNA-guided nuclease such as MG29-1, the resulting MG29-1 protein will presumably form a complex with the sgRNA in the cytoplasm before entering the nucleus in a process mediated by the nuclear localization signals that were engineered into the MG29-1 protein. This process is similar to that of delivering an mRNA and a guide RNA in a lipid nanoparticle in vivo, a method of use that is envisaged for therapeutic applications in which the HAO-1 gene would be functionally inactivated by introduction of INDELS within the coding sequence. Thus, the 42 single guide RNAs for editing activity were screened using co-transfection of mRNA and sgRNA because this method is likely a better representation of the planned therapeutic use and is thus likely to more accurately predict which of the 42 sgRNAs will be most active in a therapeutic application.

A total of 2×10⁵ Hep3B cells were plated per well of 24 well plates in growth media (EMEM plus 10% FBS) and incubated overnight in a 5% CO₂/37° C. humidified incubator. The following day, Lipofectamine MessengerMAX (Thermo Fisher) was diluted in OPTIMEM media (1.25 μL MessengerMAX plus 25 μL OPTIMEM per transfection). 300 ng of MG29-1 mRNA (0.22 pmol) and 120 ng (8.4 pmol) of sgRNA were combined in 25 μL OPTIMEM media, then mixed with 26 μL of the diluted MessengerMAX by gently flicking the tube. After incubating for 5 to 10 mins at room temperature, the RNA/MessengerMAX mixture was added to each well of Hep3B cells and mixed by swirling gently. The cells were incubated overnight (16 h), after which the media was exchanged for fresh growth media. At 48 h after addition of the RNA/MessengerMAX mixture, the cells were collected by trypsinization or by on-plate lysis, and genomic DNA was purified using either the Purelink Genomic DNA Extraction kit (Thermo Fisher) or the MagMax DNA Extraction Kit (Thermo Fisher) and the KingFisher robotic system (Thermo Fisher). The purified genomic DNA was quantified by absorbance at 260 nm. HAO-1 gene sequences targeted by the single guides were amplified by PCR from the purified genomic DNA using exon-specific primers (Table 37) and Phusion Flash High-Fidelity PCR Master Mix (Thbermo Fisher). PCR products were purified and concentrated using the DNA Clean & Concentrator 5 kit (Zymo Research), and 40 ng of PCR product was subjected to Sanger sequencing (at ELIM Biosciences) using primers located within 100 bp to 350 bp of the predicted target site for each sgRNA (Table 37). PCR products derived from untreated Hep3B cells were included as controls. The sequences of the PCR products matched the expected sequences of the HAO-1 exons.

TABLE 37
Primers designed for the human HAO1 gene,
used for PCR amplification of the
first four exons, and for Sanger sequencing.

| Target Exon | Use | Primer Name | Primer Sequence |
|---|---|---|---|
| Human HAO1 | Fwd PCR | PCR_hHe1_ | TTTCATGGATGCCCCGTTCA |
| Exon 1 | | F_+490 | |
| | Rev PCR | PCR_hHe1_ | ACGAAAAGCCAGCAGGAAG |
| | | R_−412 | A |
| | Sequencing | Seq_hHe1_ | AGCCCCAAGAACTTTTCCCT |
| | | R_−121 | |
| Human HAO1 | Fwd PCR | PCR_hHe2_ | TGCATCAGTGGTTGTCAGGG |
| Exon 2 | | F_+391 | |
| | Rev PCR | PCR_hHe2_ | CCTAGCTGTGACTTTGGGCA |
| | | R_−387 | |
| | Sequencing | Seq_hHe2_ | TGGAAAGAAGAGGAGCAGG |
| | | R_−152 | AC |
| Human HAO1 | Fwd PCR | PCR_hHe3_ | AGGCTGGATGTTCAGGTTCT |
| Exon 3 | | F_+238 | |
| | | | T |
| | Rev PCR | PCR_hHe3_ | TCCCAAAGCCAAAGCCCTTA |
| | | R_−212 | |
| | Sequencing | Seq_hHe3_ | AGCAGAAATAACTCCAGTA |
| | | F_+186 | GCCA |
| Human HAO1 | Fwd PCR | PCR_hHe4_ | GCTGGCTGAAAATCGTGTCA |
| Exon 4 | | F_+324 | A |
| | Rev PCR | PCR_hHe4_ | TCCTTGGGGCTTCTCTTTGG |
| | | R_−348 | |
| | Sequencing | Seq_hHe4_ | ACTGATTAAGACCACTAGA |
| | | F_+174 | GTATCACA |

The Sanger sequencing chromatograms were analyzed for insertions and deletions (indels) at the predicted target site for each sgRNA by an algorithm called Tracking of Indels by DEcomposition (TIDE) as described by Brinkman et al. (See. e.g., Nucleic Acids Res. 2014 Dec. 16; 42 (22): e168.Published online 2014 Oct. 9. doi: 10.10.1093/nar/gka936, which is incorporated by reference herein). As presented in Table 38, 14 guides demonstrated detectable editing at their predicted target sites. Ten guides exhibited editing activity of 10% or greater. These data demonstrate that the MG29-1 nuclease can generate RNA-guided, sequence-specific, double strand breaks in exonic regions of the human HAO-1 gene in a cultured liver cell line.

TABLE 38
Editing activity of 42 sgRNA targeting
exons 1 to 4 of the human HAO-1
gene in Hep3B cells.

| | | Average | |
|---|---|---|---|
| | | INDEL | |
| Guide Name | PAM | (Percentage)* | SD* |
| hH29-1 | TTTA | 8.5 | 4.95 |
| hH29-2 | TTTG | 15.25 | 5.56 |
| hH29-3 | TTTG | 6 | 5.66 |
| hH29-4 | TTTG | 39.5 | 10.47 |
| hH29-5 | TTTC | 0 | 0 |
| hH29-6 | TTTC | 8 | 8.49 |
| hH29-7 | TTTT | 0 | 0 |
| hH29-8 | TTTG | 2.5 | 3.54 |
| hH29-9 | TTTA | 0 | 0 |
| hH29-10 | TTTT | 0 | 0 |
| hH29-11 | TTTT | 0 | 0 |
| hH29-12 | TTTA | 0 | 0 |
| hH29-13 | TTTT | 0 | 0 |
| hH29-14 | TTTA | 0 | 0 |
| hH29-15 | TTTT | 0 | 0 |
| hH29-16 | TTTT | 0 | 0 |
| hH29-17 | TTTT | 0 | 0 |
| hH29-18 | TTTA | 3.5 | 4.95 |
| hH29-19 | TTTC | 18.75 | 12.74 |
| hH29-20 | TTTT | 6.5 | 4.95 |
| hH29-21 | TTTA | 68 | 8.04 |
| hH29-22 | TTTC | 6 | 1.41 |
| hH29-23 | TTTC | 17.5 | 2.89 |
| hH29-24 | TTTG | 10 | 4.24 |
| hH29-25 | TTTG | 0 | 0 |
| hH29-26 | TTTT | 0 | 0 |
| hH29-27 | TTTA | 0 | 0 |
| hH29-28 | TTTC | 0 | 0 |
| hH29-29 | TTTT | 0 | 0 |
| hH29-30 | TTTA | 0 | 0 |
| hH29-31 | TTTC | 0 | 0 |
| hH29-32 | TTTT | 0 | 0 |
| hH29-33 | TTTT | 0 | 0 |
| hH29-34 | TTTG | 0 | 0 |
| hH29-35 | TTTA | 0 | 0 |
| hH29-36 | TTTT | 0 | 0 |
| hH29-37 | TTTC | 0 | 0 |
| hH29-38 | TTTA | 0 | 0 |
| hH29-39 | TTTT | 0 | 0 |
| hH29-40 | TTTG | 0 | 0 |
| hH29-41 | TTTG | 34 | 13.04 |
| hH29-42 | TTTG | 0 | 0 |

*Data are the mean of 2 independent transfections

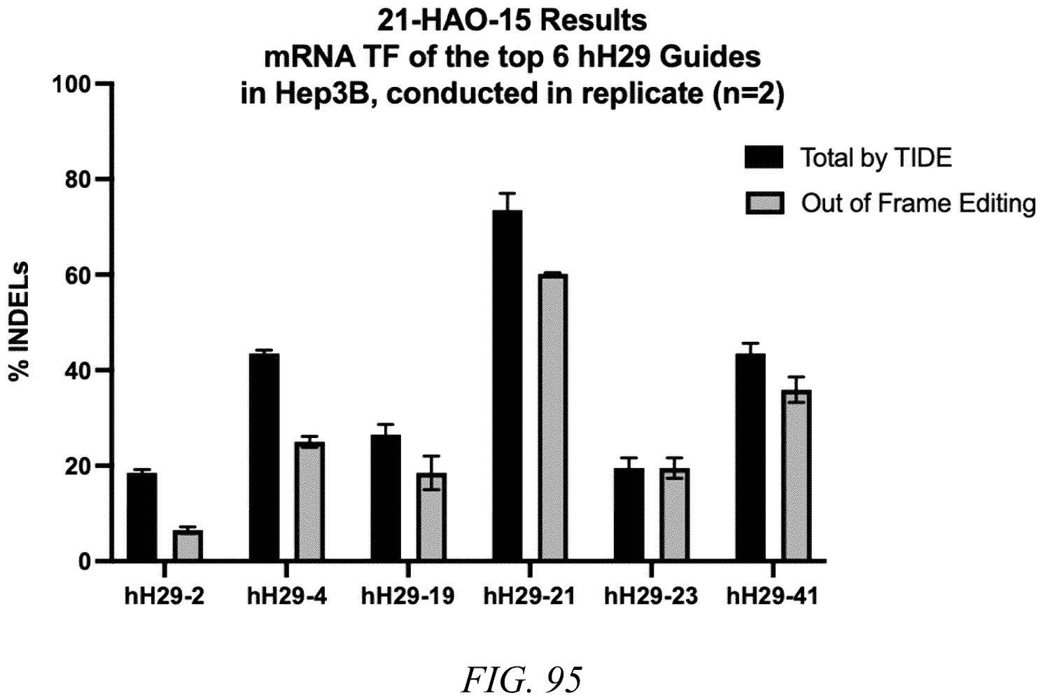

Six of the sgRNAs (hH29-2, hH29-4, hH29-19, hH29-21, hH29-23, and hH29-41) with the highest editing activity from this initial screen were re-tested using the same MG29-1 mRNA/sgRNA MessengerMax transfection method in Hep3B cells. Two independent transfections were performed, and indel frequency was determined using the same Sanger sequencing method described above, followed by analysis using TIDE. The mean indel frequencies (FIG. 95) ranged from 20% to 75%. The rank order of editing efficiency was hH29-21>hH29-41>hH29-4>hH29-19>hH29-23=hH29-2. An evaluation of the frequency of indels that generate a frame shift (Out of Frame Editing) was also made based on the Sanger Chromatograms, and these results are plotted in FIG. 95. The ratio of out of frame edits to total indels was different for different sgRNAs, but the rank order of out of frame editing frequency was the same as total indels.

Four of the sgRNAs (hH29-21, hH29-4, hH29-41, and hH29-23) with the highest editing activity in Hep3B cells were also evaluated for their editing activity by nucleofection of ribonucleoprotein particles (RNP) into both Hep3B cells and another human liver-derived cell line, HuH7. The ribonuclear protein complex was formed by mixing 160 pmol of sgRNA and 126 pmol purified MG29-1 protein in PBS buffer. A total of 2×10⁵ Hep3B or HuH7 cells in suspension in complete SF nucleofection reagent (Lonza) were nucleofected with the pre-formed nuclease/sgRNA complex using a 4D nucleofection device (Lonza). After nucleofection, the cells were plated in 24 well plates in growth media plus 10% FBS and incubated in a 5% CO₂ incubator for 48 h to 72 h. Genomic DNA was then extracted from the cells using a column based purification kit (Purelink genomic DNA mini kit, ThermoFisher Scientific) and quantified by absorbance at 260 nm. HAO-1 gene sequences targeted by the single guides were amplified by PCR from the purified genomic DNA using the relevant exon-specific primers (Table 37) and Phusion Flash High-Fidelity PCR Master Mix (Thermo Fisher). PCR products were purified and concentrated using DNA Clean & Concentrator 5 (Zymo Research), and 40 ng of PCR product subjected to Sanger sequencing (at ELIM Biosciences) using relevant primers (Table 37) located within 100 to 350 bp of the predicted target site for each sgRNA. PCR products derived from untransfected cells were included as controls. The sequence of the PCR products matched the expected sequences of the HAO-1 exons. The Sanger sequencing chromatograms were analyzed for insertions and deletions (indels) at the predicted target site for each guide by Tracking of Indels by DEcomposition (TIDE) as described by Brinkman et al. (Nucleic Acids Res. 2014 Dec. 16; 42 (22): e168.Published online 2014 Oct. 9. doi: 10.1093/nar/gka936). The presence of indels at the target site is the consequence of the generation of double strand breaks in the DNA, which are then repaired by the error-prone cellular repair machinery which introduces insertions and deletions. The results (FIG. %) demonstrate that using the more efficient transfection method of RNP nucleofection, the editing frequency for these top 4 guides ranged from 25% to 95%. The rank order of editing activity for these 4 guides was hH29-21>hH29-4>hH29-41>hH29-23, which was similar but not identical to the rank order of editing activity measured by transfection of mRNA and sgRNA by MessengerMAX in Hep3B cells (FIG. 95).

### Example 73—Editing Activity of the Most Active MG29-1 Guides Targeting Exons 1 to 4 of Human HAO-1 in Primary Human Hepatocytes

Primary human hepatocytes (PHH) are hepatocytes that are isolated from the livers of deceased humans using documented methods such as Kegel et al. (doi: 10.3791/53069). PHH are the closest cell-based model for human hepatocytes in their native state in vivo and thus represent an in vitro cell system that can be used to predict the performance of gene editing systems in humans in vivo. PHH have undergone minimal manipulation, do not undergo cell division, and have a limited lifespan in culture of about 7 days. PHH were obtained from commercial suppliers (Lonza, Gibco) and cultured according to the protocols provided by the supplier. To evaluate the editing activity of the four most active MG29-1 guides targeting human HAO-1, the sgRNAs with spacers 4, 21, 23, and 41 were chemically synthesized with the incorporation of chemical modification #37 to the backbone and the nucleobases that improve the stability and activity of the sgRNA. The 4 sgRNAs with chemical modifications #37 are designated as hH29-4-37 (SEQ ID 5831), hH29-21-37 (SEQ ID 5832), hH29-23-37 (SEQ ID 5833), and hH29-41-37 (SEQ ID 5834). 1028 ng of MG29-1 mRNA and 222 ng of each single guide RNA (1:20 molar ratio of mRNA:guide RNA) were transfected into primary human hepatocyte (PHH) cells as follows. One day prior to transfection, PHH cells were thawed and seeded in 1.0 ml HBM™-5% FBS-HCM™ SingleQuot Supplements media into collagen-treated 12 well plates at 1,000,000 viable cells per well. On the day of transfection, 60.4 μL of OptiMEM media and 2.1 μL of Lipofectamine MessengerMax Solution (Thermo Fisher) were mixed in a master mix solution, vortexed, and allowed to sit for at least 10 minutes at room temperature. In separate tubes, 1028 ng of MG29-1 mRNA and 222 ng of the sgRNA were mixed, brought to a volume of 62.5 μL with OptiMEM media, and pipetted briefly. The appropriate volume of MessengerMax solution was added to each RNA solution, mixed by flicking the tube, and briefly spun down at a low speed. The complete editing reagent solutions were allowed to incubate for at least 10 minutes at room temperature, then added directly to the PHH cells. Following the transfection, media was replaced every day until harvest. Three days post-transfection, the culturing media was aspirated from each well of PHH cells and replaced with MagMAX™ Cell and Tissue DNA Extraction Buffer (Thermo Fisher). Cells were scraped and transferred to a 96-well plate, and genomic DNA was purified by automated magnetic bead purification via the KingFisher Flex with the MagMAX™ DNA Multi-Sample Ultra 2.0 Kit (Thermo Fisher).

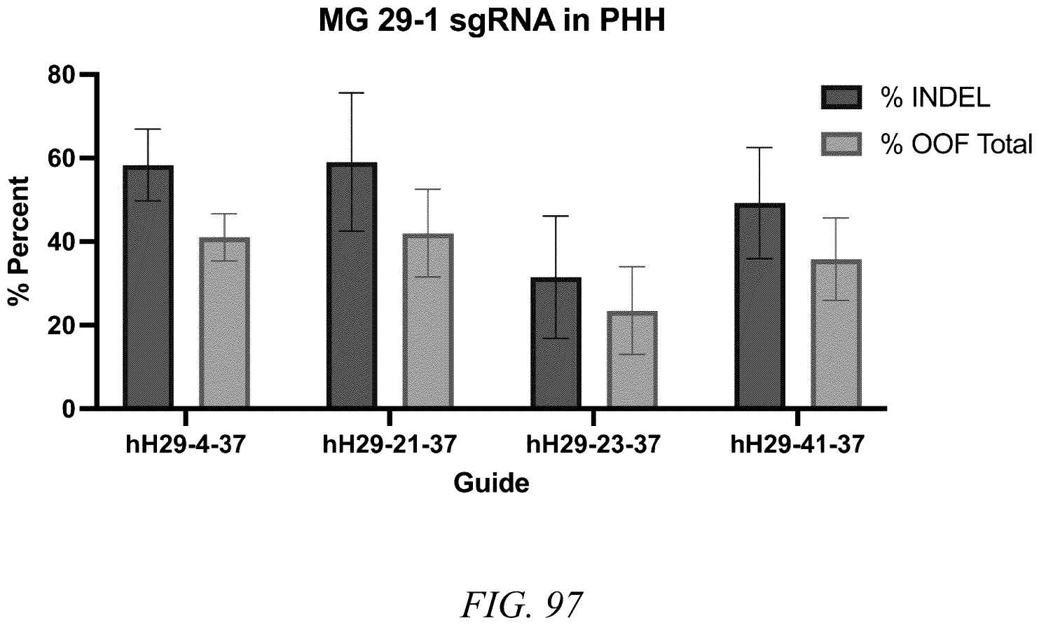

The region of the HAO-1 gene targeted by each specific sgRNA was PCR amplified with Q5 high fidelity DNA polymerase and exon specific primers (Table 37) but with the addition of adapters complementary to the barcoded primers used for next generation sequencing (NGS) for a total of 29 cycles. The product of this first PCR reaction was PCR amplified using the barcoded primers for NGS using a total of 10 cycles. The resulting product was subjected to NGS on an Illumina MiSeq instrument, and the results were processed using a custom script to generate the percentage of sequencing reads that contain insertions or deletions (indels) at the targeted site in the HAO-1 gene. Two independent transfections of PHH were performed, and in each experiment, each of the sgRNA were tested in duplicate wells that were separately assayed for indels by NGS. The average of the indel frequency in the 2 wells was then calculated. The mean of indel frequency from the 2 independent experiments was then determined (Table 39) and also shown in graph format (FIG. 97), in which the error bars represent the standard deviation.

TABLE 39
Editing activity in PHH of 4 MG29-1 sgRNA targeting human HAO-1

| sgRNA | Exon | Mean total INDEL %* | Stdev |
|---|---|---|---|
| hH29-4-37 | 1 | 58.3 | 5.6 |
| hH29-21-37 | 2 | 59.1 | 10.5 |
| hH29-23-37 | 3 | 31.5 | 10.5 |
| hH29-41-37 | 4 | 49.2 | 9.9 |

*data are the mean of 2 independent transfection experiments each performed in duplicate wells

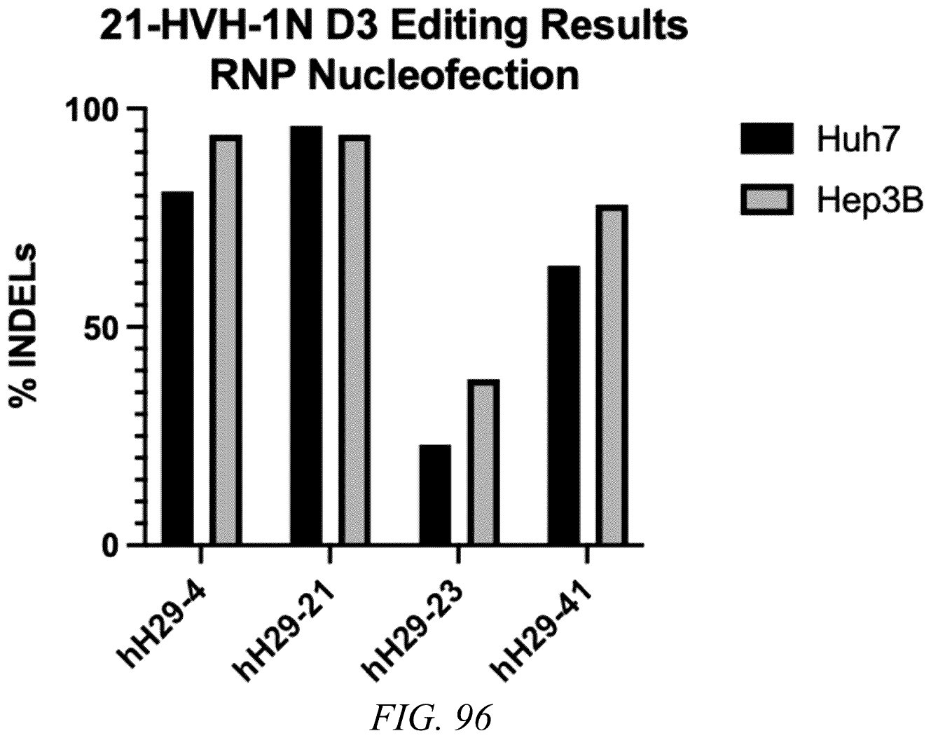

The results indicate that all four guides edited the HAO-1 gene in PHH with mean INDEL frequencies ranging from 20% to 58% (FIG. 97). Guides hH29-4-37 and mH29-21-37 exhibited the highest editing activity in PHH. Guide hH29-41-37 was slightly less active than guides hH29-4-37 and mH29-21-37, but the difference was not significant. Guide hH29-23-37 was the least active of the 4 guides in PHH. Guide hH29-23-37 was also the least active of these 4 guides in Hep3B and HuH7 cells (Table 37, FIG. 95, FIG. 96). PHH are a surrogate for editing hepatocytes in vivo. These data demonstrate that the MG29-1 nuclease and an appropriate sgRNA have utility in generating insertions and deletions in the coding sequence of the human HAO-1 gene, which are expected to generate a mixture of non-sense, missense, and deletion mutations leading to disruption of GO protein expression and/or activity.

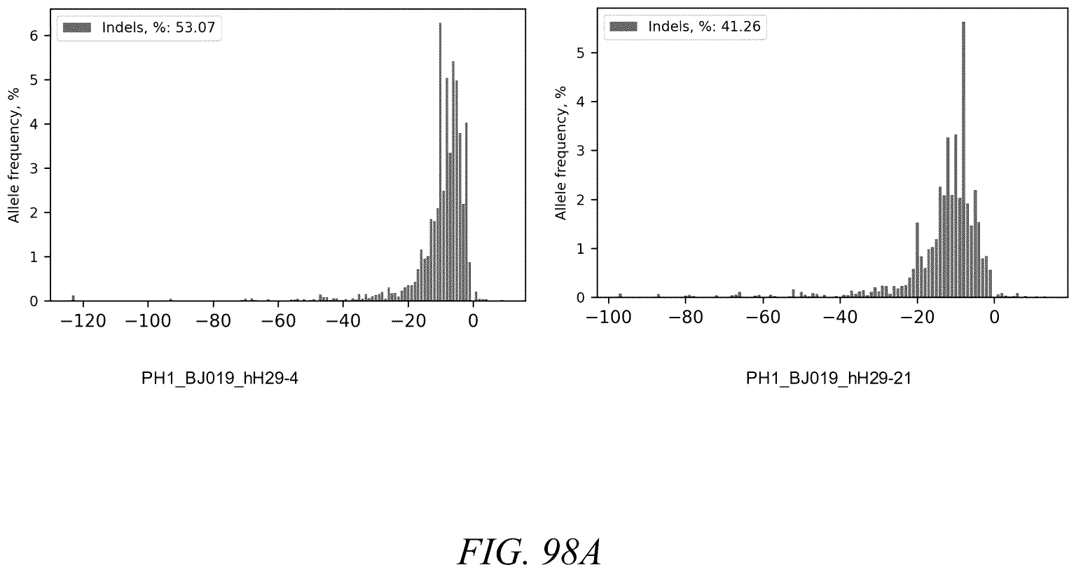

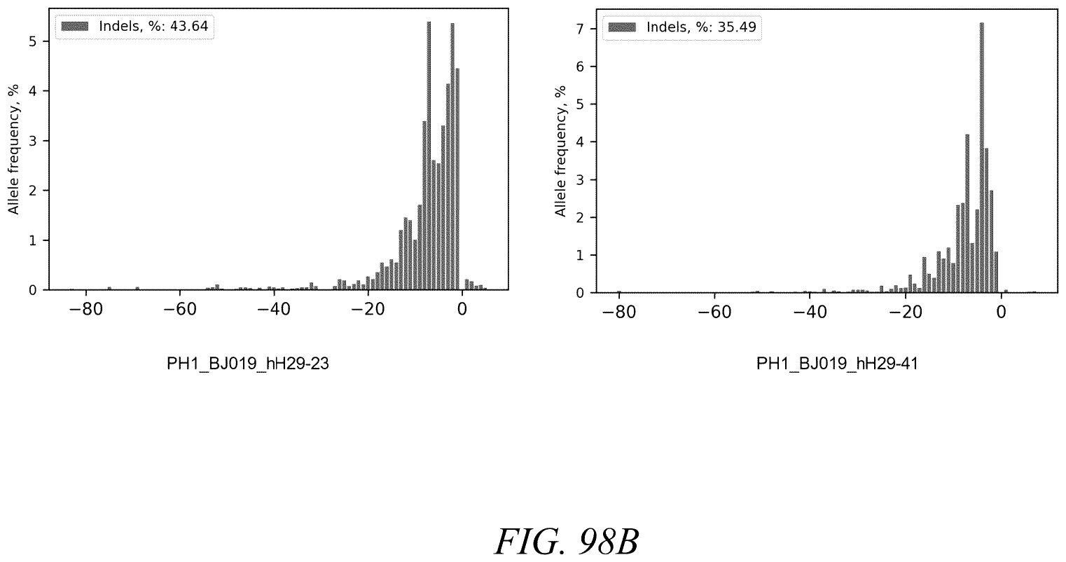

The indel profile obtained from the same NGS sequence data of the HAO-1 gene in PHH transfected with MG29-1 mRNA and the 4 sgRNAs was used to determine the percentage of INDELS that result in a frame shift. Sequence reads in which the number of bases inserted or deleted at the target site are not 3 bases or a multiple of 3 bases will shift the reading frame of the HAO-1 protein coding sequence. A frame shift will alter the amino acid sequence encoded in the mRNA downstream of the indel and in many cases will introduce an in frame stop codon at some point (this can be predicted for each allele). The NGS data (comprising several thousand reads for each sample) was analyzed using a Python script that calculates the percentage of total sequencing reads in which there is an indel that created a frame shift, and this was designated as the out of frame (OOF) indel percentage. The OOF indel percentage is plotted in FIG. 97 alongside the total indel percentage. The OOF indel percentage ranged from 20% to 40%, which represents between 70% and 80% of the total INDEL percentage, demonstrating that the majority of indels are predicted to create a frameshift. The in-frame indels will delete 1 or more codons from the mRNA, and thus will remove 1 or more amino acids from the glycolate oxidase protein that is encoded by HAO-1. FIGS. 98A and 98B show representative indel profiles for each of the 4 MGf29-1 sgRNAs from edited PHH. In-frame deletions of 3, 6, 9, 12, 15, 18, and 21 bases are evident at different relative frequencies. An analysis of the frequencies of the in-frame deletions and their impact on the GO protein sequence can be used to inform selection of sgRNA that will result in the greatest reduction in GO protein function.

### Example 74—In Vivo Editing Activity of Single Guide RNAs for MG29-1 with 22 and 20 Nucleotide Spacers that Target the Exonic Regions of Mouse HAO-1

To evaluate the ability of the MG29-1 Type V nuclease to edit the genome in vivo in a living animal, a lipid nanoparticle was used to deliver an mRNA encoding the MG29-1 nuclease and one of four guide RNAs. The ability of MG29-1 with sgRNA comprising 22 nucleotide (nt) spacers to edit the mouse HAO-1 locus in the liver of mice when delivered in an LNP was demonstrated in Example 55. Experiments in cultured mammalian cells demonstrated that reducing the length of the spacer region in MG29-1 sgRNA from 22 nt to 20 nt did not change editing activity. Reducing the spacer from 22 nt to 20 nt may be advantageous in terms of minimizing off-target activity and in terms of sgRNA manufacture. In order to validate that MG29-1 sgRNA with a reduced spacer length of 20 nt retained potency in vivo, four guide RNAs (mH29-29-37, mH29-29-44, mH29-29 s-37, and mH29-29s-44) were designed and tested in mice. The sequences of these guides are shown below in Table 40.

TABLE 40
Sequences and chemical modifications of
guide RNAs tested in vivo in mouse study

| Guide RNA | Sequence |
|---|---|
| mH29-29-37 | mC*mU*mU*U*UAAUUmUmCmUmACU*G*U |
| | *U*GUAGAU CCUUAGGfAfGfAfAfAfAf |
| | UfGfCfC*fA*fAfA*fU*mC |
| mH29-29-44 | mC*mU*mU*U*U*AAUUmUmCmUmACU*G* |
| | U*U*GUAGAU CCUUAGGfAfGfAfAfAfA |
| | fUfG*fCfC*fA*fA*fA*fU*mC |
| mH29-29s-37 | mC*mU*mU*U*UAAUUmUmCmUmACU*G*U |
| | *U*GUAGAU CCUUAGGfAfGfAfAfAfAf |
| | UfG*fCfC*fA*fA*MA |
| mH29-29s-44 | mC*mU*mU*U*UAAUUmUmCmUmACU*G*U |
| | *U*GUAGAU CCUUAGGfAfGfAfAfAfAf |
| | UfG*fC*fC*fA*fA*MA |

Notations for chemical modifications: m= 2′O-Methyl ribonucleotide (e.g mC= cytosine ribonucleotide with 2′-O Methyl in place of 2′ hydroxy1); f= 2′Fluorine ribonucleotide (e.g fC = cytosine ribonucleotide with 2′ fluorine in place of 2′ hydroxyl); *= phosphorothioate bond. All other bases are native ribonucleotides.
Backbone sequence in normal type.
Spacer sequence in bold type.

Guides mH29-29-37 and mH29-29-44 have the same nt sequence (spacer 29) but different chemical modifications (chemistries 37 and 44), while guides mH29-29s-37 and mH29-29s-44 have spacer sequences shortened by two nts at the 3′ end but are otherwise identical in nt sequence to mH29-29-37 and mH29-29-44, respectively. The locations of the 2′-fluoro, 2′-O-methyl, and phosphorothioate modifications in the spacer region of the guides with a 20 nt spacer are shifted relative to those in the corresponding guides with a 22 nt spacer. Because the chemical modifications in the sgRNA impact sgRNA stability, which is critical for in vivo potency, it was important to evaluate the impact of these changes on in vivo editing.

Preparation of MG29-1 mRNA

The mRNA encoding MG29-1 (SEQ ID NO: 5846) was generated by in vitro transcription of a linearized plasmid template using T7 RNA polymerase and standard conditions using nucleotides and enzymes purchased from New England Biolabs or Trilink Biotechnologies. The protein coding sequence of the MG29-1 cassette comprises the following elements from 5′ to 3′: the nuclear localization signal from SV40, a five amino acid linker (GGGS), the protein coding sequence of the MG29-1 nuclease from which the initiating methionine codon was removed, a 3 amino acid linker (SGG), and the nuclear localization signal from nucleoplasmin. The DNA sequence of this cassette was codon optimized for human using a commercially available algorithm. An approximately 100 nucleotide polyA tail was encoded in the plasmid used for in vitro transcription and the mRNA was co-transcriptionally capped using the CleanCAP™ reagent purchased from Trilink Biotechnologies. Uridine in the mRNA was replaced with N1-methyl pseudouridine.

### Preparation of Lipid Nanoparticles

The lipid nanoparticle (LNP) formulation used to deliver the MG29-1 mRNA and the guide RNA is based on LNP formulations described in the literature including Kauffman et al. (Nano Lett. 2015, 15, 11, 7300-7306 https://doi.org/10.1021/acs_nanolett_5b024970) The four lipid components were dissolved in ethanol and mixed in an appropriate molar ratio to make the lipid working mix. The RNAs were diluted in 100 mM Sodium Acetate (pH 4.0) to make the RNA working stocks. The lipid working stock and the RNA working stocks were mixed in a microfluidics device (Ignite NanoAssembler, Precision Nanosystems) at a flow rate ratio of 1:3, respectively, and a flow rate of 12 mL/min. The LNP were dialyzed against phosphate buffered saline (PBS) for 2 to 16 hours and then concentrated using Amicon spin concentrators (Milipore) until the desired volume was achieved. The concentration of RNA in the LNP formulation was measured using the Ribogreen reagent (Thermo Fisher). The diameter and polydispersity (PDI) of the LNP were determined by dynamic light scattering. Typical LNP had diameters ranging from 70 nm to 84 nm and PDI of 0.098 to 0.150.

### Mouse Dosing and Harvesting

LNP for mRNA and sgRNA were mixed at 1:1 mass ratio and injected intravenously into 7 week-old C57B16 wild type mice via the tail vein (0.1 mL per mouse) at a total RNA dose of 0.84 mg RNA per kg body weight. Fourteen days after dosing, the mice were sacrificed, and the left lateral, medial, and right lateral lobes of the liver were collected for preparation of DNA, RNA, and protein, respectively. Blood was collected by exsanguination via cardiac puncture and collected onto BD microtainer (heparin coated). Samples were kept on wet ice for no longer than 30 minutes prior to centrifugation. Samples were centrifuged at 2,000 G for 10 minutes and the plasma transferred to 1.5 mL Cryotubes and stored at −80° C.

### Genomic DNA Preparation and Editing Analysis by Next-Generation Sequencing (NGS

The left lateral lobe of the liver (100 mg) was homogenized using a Bead Ruptor (Omni International) in the digestion buffer supplied in the PureLink Genomic DNA Isolation Kit (Thermo Fisher Scientific). Genomic DNA was purified from the resulting homogenate using the MagMAX™ DNA Multi-Sample Ultra 2.0 Kit (Applied Biosystems) and quantified by measuring the absorbance at 260 nm. Genomic DNA purified from mice injected with PBS buffer alone was used as a control. The region of the HAO-1 gene targeted by each specific sgRNA was PCR amplified with Q5 high fidelity DNA polymerase and gene specific primers (Table 41) with adapters complementary to the barcoded primers used for next generation sequencing (NGS) for a total of 29 cycles.

TABLE 41
Primers used to amplify HAO1 guide
target site and for NGS

| | | Fwd with | Rev with |
|---|---|---|---|
| | Primer | Miseq | Miseq |
| Guide | Set Name | adapter | adapter |
| mH29-29 | mHAO1- | GCTCTTCCGA | GCTCTTCCGATC |
| (spacer | NGS-P3 | TCTNNNNNGT | TNNNNNTGTAG |
| 29) | | GATGTCAATC | GTGGCTGAGTA |
| | | GTCTGAGC | CGTT |

The product of this first PCR reaction was PCR amplified using the barcoded primers for NGS for a total of 10 cycles. The resulting product was subjected to NGS on an Illumina MiSeq instrument, and the results were processed using a custom Python script to generate the percentage of sequencing reads that contain insertions or deletions (INDELS) at the targeted site in the HAO-1 gene.

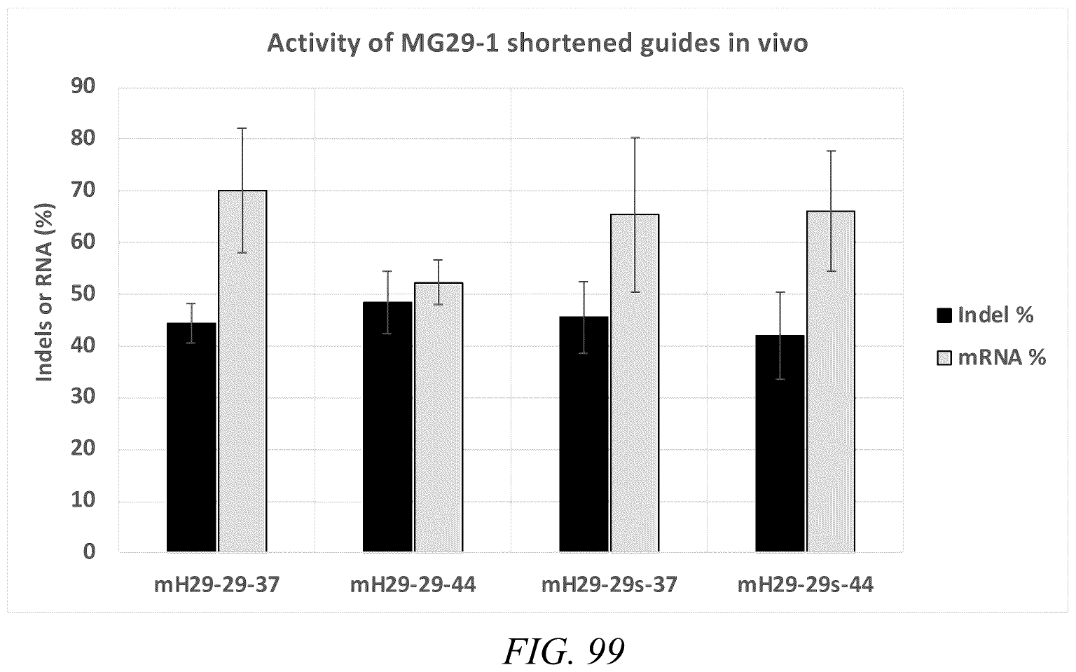

NGS analysis showed that editing of the groups dosed with LNP encapsulating MG29-1 mRNA and each of the four sgRNA ranged from 42% to 48% of total liver genomic DNA (FIG. 99). There were only slight differences between the groups, indicating that the sgRNA with the 20-nt spacer edited the target locus as efficiently as the 22-nt spacer, and that guide RNAs with chemistries 37 and 44 edited equally well.

### RNA Preparation and Analysis by RT-ddPCR

The medial lobe of the liver was stored in RNAlater or RNAprotect (Qiagen) to preserve the integrity of the RNA prior to homogenization. A maximum of 10 mg of tissue was transferred to a 2 mL tube containing 1.4 mm ceramic beads and homogenized using the Bead Ruptor Elite (OMNI International) following the soft tissue homogenization protocol, 5.00 m/s for 10-15 sec. Homogenized tissue was then processed using RNeasy Protect Kit (Qiagen) with an additional 45 minute on-column DNase I digestion treatment (Qiagen). The RNA products were quantified by measuring the absorbance at 260 nm. Isolated RNA samples then underwent an additional DNase treatment using the ezDNase™ Enzyme (Invitrogen) and reverse transcription using the SuperScript™ IV Vilo Master Mix (Invitrogen). The cDNA product was then quantified by measuring the absorbance at 260 nm.

HAO-1 mRNA levels were quantified using a custom ddPCR probe-based assay that was multiplexed with the housekeeping gene GAPDH. HAO-1 was labeled with a HEX probe while GAPDH was labeled with a FAM probe. Both probes were flanked by primers specific to the respective genes creating amplicons of about 115 bp. The template material for the assay was 100 ng of cDNA product from the process above mixed with 900 nM of each primer and 250 nM of probe per gene assay along with 10 μL of ddPCR Supermix for Probes (No dUTP) (Bio-Rad Laboratories) raised to a final volume of 20 μL with nuclease-free water. The PCR mix was parsed into thousands of oil droplets via the AutoDG ddPCR System (Bio-Rad Laboratories), ran in the C1000 Touch™ deep well thermal cycler (Bio-Rad Laboratories), and analyzed using the QX200 Droplet Reader (Bio-Rad Laboratories). Results were divided into four sections: negative droplets (no fluorescence), single positive droplets (HEX or FAM positive fluorescence), and double positive droplets (both HEX and FAM fluorescence). Using the QuantaSoft Software (Bio-Rad Laboratories), copies per μL of HAO1 and GAPDH were calculated for each sample. The ratio of HAO1 to GAPDH was compared for mice treated with mH29-29 against mice treated with buffer only. The GAPDH-normalized levels of HAO1 mRNA of the mH29-29 treated groups ranged from 52% to 70% of the control group (FIG. 99) and correlated with the editing efficiency as defined by INDEL %, indicating little or no effect of spacer length or chemistry upon guide RNA activity.

TABLE 42
Primers and probes used in the amplification
HAO1 and GAPDH ddPCR assays

| | | | Fwd | Rev |
|---|---|---|---|---|
| Target | Probe | Probe | Primer | primer |
| Gene | Fluorescence | Sequence | Sequence | sequence |
| HAO1 | HEX | AGTG+GGTG+ | GGGGAGA | CTCACCA |
| | | C+CA+G | AAGGTGT | ATGTCTTG |
| | | AAT+GTGAA | TCAAGATGT | TCGATGA |
| GAPDH | FAM | CATGACCA+ | GCACCACC | CCATCCAC |
| | | CAGT+C+ | AACTGCT | AGTCTTCT |
| | | CATGCCATC | TAG | GGG |

Notations for chemical modifications: += Locked Nucleic Acid (LNA) base modification

### Example 75—Investigation of Different mRNA:sgRNA Ratios and LNP Formulation Procedures on Editing Efficiency In Vivo in Mice

In this in vivo mouse study, MG29-1 mRNA and sgRNA mH29-29-50b were formulated separately into LNP and then mixed at different mass ratios of mRNA and sgRNA prior to dosing. The same mRNA and sgRNA were also co-formulated in the same LNP at a 1:1 mass ratio then either kept at 4° C. and dosed within 48 h (fresh LNP), or frozen and stored at −80° C., then thawed and dosed within 2 h (frozen LNP). The sequence of the mH29-29-50b sgRNA is shown in Table 43. This guide has a 20-nt spacer sequence and the chemical modifications designated chemistry 50.

TABLE 43
Sequences and chemical modifications of
guide RNAs tested in vivo in
mouse studies

| Guide RNA | Sequence |
|---|---|
| mH29-29-50 | mG*mU*mU*GAGAAUC*mG* |
| | mA*mA*mAGAUUCUCAAC*m |
| | C*mU*mU*U*UAAUUmUmCm |
| | UmACU*G*U*U*GUAGAUCC |
| | UUAGGfAfGfAfAfAfAfUf |
| | GfCfC*fA*fAfA*fU*mC |
| mH29-29-50b | mG*mU*mU*GAGAAUC*mG* |
| | mA*mA*mAGAUUCUCAAC*m |
| | C*mU*mU*U*UAAUUmUmCm |
| | UmACU*G*U*U*GUAGAUCC |
| | UUAGGfAfGfAfAfAfAfUf |
| | GfCfC*fA*fA*MA |
| mH29-29-51b | mG*mU*mU*GAGAAUC*mG* |
| | mA*mA*mAGAUUCUCAAC*m |
| | C*mU*mU*U*UAAUUmUmCm |
| | UmACU*G*U*U*GUAGAUCC |
| | UUAGGAGAAAAUGCC*A*mA |
| | *mA |
| mH29-29-52b | mG*mU*mU*GAGAAUC*mG* |
| | mA*mA*mAGAUUCUCAAC*m |
| | C*mU*mU*U*UAAUUmUmCm |
| | UmACU*G*U*U*GUAGAUCC |
| | UUAGGAfGAfAAfAUfG*C* |
| | fCA*fA*mA |
| mH29-29-53b | mG*mU*mU*mG*mA*mG*mA |
| | *mA*mU*mC*mG*mA*mA*m |
| | A*mG*mA*mU*mU*mC*mU* |
| | mC*mA*mA*mC*mC*mU*mU |
| | *U*UAAUUmUmCmUmACU*G |
| | *U*U*GUAGAUCCUUAGGfA |
| | fGfAfAfAfAfUfGfCfC*f |
| | A*fA*mA |
| mH29-29-54b | mG*mU*mU*mG*mA*mG*mA |
| | *mA*mU*mC*mG*mA*mA*m |
| | A*mG*mA*mU*mU*mC*mU* |
| | mC*mA*mA*mC*mC*mU*mU |
| | *U*UAAUUmUmCmUmACU*G |
| | *U*U*GUAGAUCCUUAGGAG |
| | AAAAUGCC*A*mA*mA |

Notations for chemical modifications: m= 2′O-Methyl ribonucleotide (e.g mC= cytosine ribonucleotide with 2′-O Methyl in place of 2′ hydroxy1); f= 2′Fluorine ribonucleotide (e.g fC= cytosine ribonucleotide with 2′ fluorine in place of 2′ hydroxyl); *= phosphorothioate bond.
All other bases are native ribonucleotides.
Backbone sequence in normal type.
Spacer sequence in bold type.

### Preparation of Lipid Nanoparticles

The LNP formulations used to deliver the MG29-1 mRNA and the guide RNA were prepared by the same procedure as in Example 74 with the following differences:
1. For different ratios of separately-formulated LNP, the mRNA and guide RNA LNP were mixed at 1:2,1:1.5,1:1, 1:0.75, and 1:0.5 mRNA:guide RNA mass ratios.
2. For the co-formulated LNP, the mRNA and the guide RNA were mixed prior to formulation at a 1:1 mass ratio and stored overnight at either 4° C. (“Fresh”) or −80° C. (“Frozen”).

### Mouse Dosing and Harvesting

mRNA and sgRNA formulated in separate LNP were mixed at different mass ratios as above and injected intravenously into 7 week-old C57B16 wild type mice via the tail vein (0.1 mL per mouse) at a total RNA dose of 0.35 mg RNA per kg body weight. Co-formulated LNPs were injected similarly at the same dose. Seven days after dosing, the mice were sacrificed, and the left lateral, medial, and right lateral lobes of the liver were collected for preparation of DNA, RNA, and protein, respectively, by the same procedure as in Example 74. Terminal blood was collected by exsanguination via cardiac puncture as per the procedure of Example 74.

### Genomic DNA Preparation and Editing Analysis by NGS

Genomic DNA was prepared and analyzed by NGS as in Example 74.

### RNA Preparation and Analysis by RT-ddPCR

RNA was prepared and analyzed by RT-ddPCR as in Example 74.

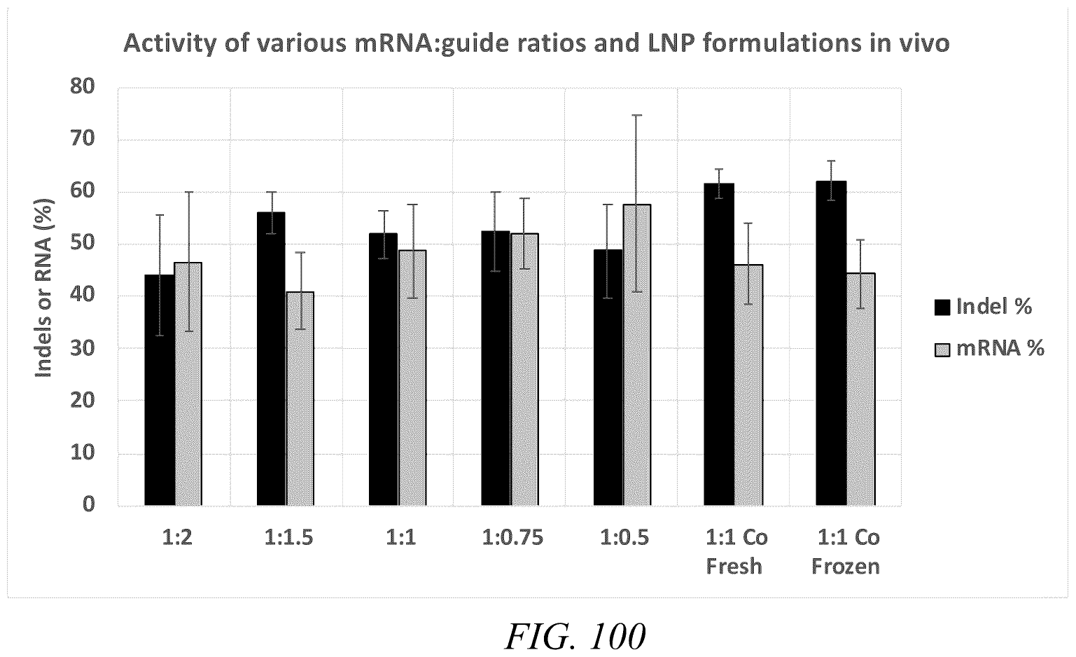

The results demonstrated that the ratio between the mRNA and guide in the separately-formulated LNPs does not greatly affect the editing efficiency (FIG. 100). Although there was somewhat higher editing efficiency at the middle three ratios (1:1.5, 1:1, and 1:0.75) than at the extremes (1:2 and 1:0.5), the differences were within the variation of the experiment. The co-formulated LNPs resulted in higher editing than the separate formulations, with the fresh and frozen LNPs performing equally well. Similar to the editing efficiency, there was little difference in the amount of HAO-1 mRNA present in the livers of mice treated with separately-formulated LNPs at different MG29-1 mRNA:guide ratios (FIG. 100). HAO-1 mRNA levels in the mice treated with the co-formulated LNPs were reduced more than 50%, similar to the groups that received the separately-formulated LNPs. In conclusion, these results demonstrate that the MG29-1 mRNA and sgRNA with chemistry 50 can be co-formulated into LNP with no reduction in editing potency and that a range of mRNA to sgRNA ratios between 1:2 to 1:0.5 can be used.

### Example 76—Investigation of the Impact of Different Guide Chemistries Upon Editing Efficiency In Vivo

### Preparation of Lipid Nanoparticles

The LNP formulations used to deliver the MG29-1 mRNA and the guide RNA were prepared by the same procedure as in Example 74. Guide RNA sequences and chemistries are listed in Table 40.

### Mouse Dosing and Harvesting

mRNA and sgRNA were formulated separately into LNP then mixed at 1:1 mass ratios as in Example 74 and injected intravenously into 7 week-old C57B16 wild type mice via the tail vein (0.1 mL per mouse) at a total RNA dose of 0.25 mg RNA per kg body weight. Seven days after dosing, the mice were sacrificed, and the left lateral, medial, and right lateral lobes of the liver were collected for preparation of DNA, RNA, and protein, respectively, by the same procedure as in Example 74. Terminal blood was collected by exsanguination via cardiac puncture as per the procedure of Example 74.

### Genomic DNA Preparation and Editing Analysis by NGS

Genomic DNA was prepared and analyzed by NGS as in Example 74.

### RNA Preparation and Analysis by RT-ddPCR

RNA was prepared and analyzed by RT-ddPCR as in Example 74.

All guides designated with a “b” in the guide name (e.g. mH29-29-50b) contain 20-nt spacers. Guides mH29-29-37 and mH29-29-50 contain 22-nt spacers. Guides with chemistries 51, 52, 53, and 54 contain the same nucleotide sequence as the guide with chemistry 50 but have differences in the chemical modifications at specific regions of the guide. Chemistry 51 is identical to chemistry 50 with the exception of the removal of the 2′-fluoro modifications in the spacer. Chemistry 52 is identical to chemistry 50 with the exception of the removal of half of the 2′-flouro modifications in the spacer. Chemistry 53 is identical to chemistry 50 with the exception of an additional 21 phosphorothioate linkages and 21 2′-O methyl modifications. Chemistry 53 is identical to chemistry 50 with the exception of an additional 21 phosphorothioate linkages and 21 2′-O methyl modifications in the 5′ stem loop. Chemistry 54 is identical to chemistry 50 with the exception of an additional 21 phosphorothioate linkages and 21 2′-O methyl modifications in the 5′ stem loop and the removal of the 2′-fluoro modifications in the spacer.

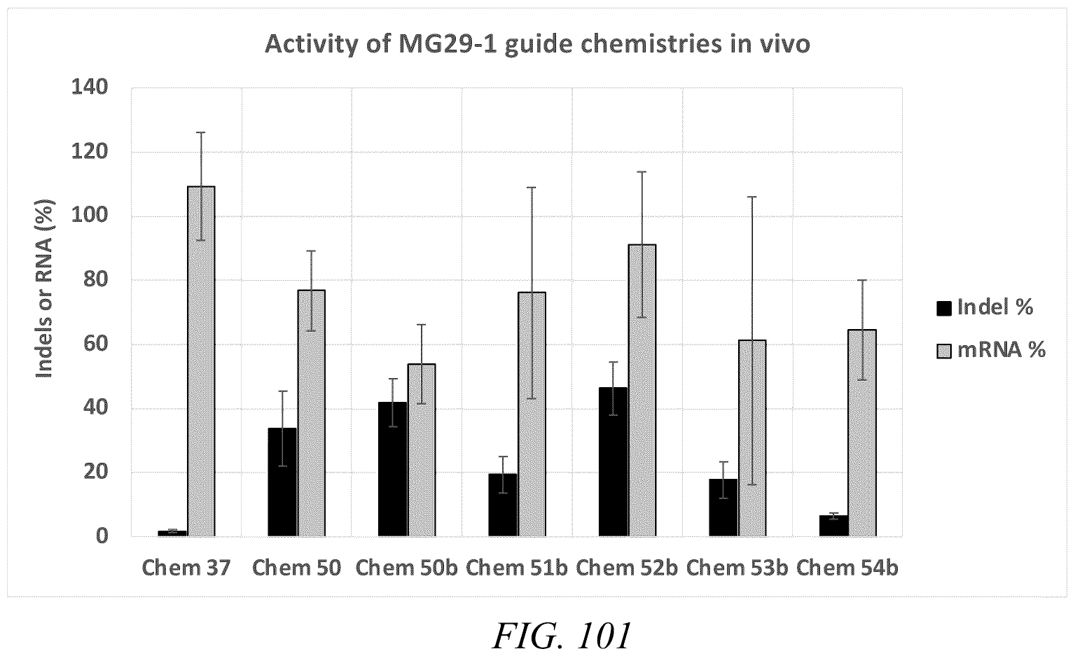

The results indicate that guide RNAs with chemistry 50 (50b) and chemistry 52 (52b) had the highest editing efficiency (FIG. 101). The introduction of INDELS in the HAO-1 gene is expected to reduce the level of HAO-1 mRNA through nonsense-mediated mRNA decay, due to reading frame shifts and the resulting premature stop codons. Treatment with the 22-nt guide with chemistry 50 (mH29-29-50b) resulted in the lowest level (largest reduction) of HAO-1 mRNA, consistent with this mechanism (FIG. 101). Chemistry 51 (51b) was 2-fold less potent than chemistry 50 (50b), indicating that the 2′-fluoro modifications in the spacer contributed significantly to potency. Chemistry 52 (52b) had similar or slightly improved editing potency compared to chemistry 50 (50b), indicating that in the context of this guide spacer sequence, it was possible to remove half of the 2-fluoro modifications in the spacer without significantly reducing potency. Chemistry 53 (53b) had about 2-fold lower editing potency compared to chemistry 50 (50b), indicating that the addition of 2′-O-methyl and PS linkages in the 5′ stem-loop had a negative impact on potency, which is surprising given that these additional modifications were expected to improve guide stability. Chemistry 54 (54b) had about 4-fold lower potency compared to chemistry 50 (50b) and about 2-fold lower editing potency compared to chemistry 53 (53b), confirming that the additional 2′O-methyl and PS bases in the 5′ stem loop and the removal of the 2′-fluoro bases in the spacer both had an additive negative effect on guide potency.

### Example 77—Gene Editing Outcomes at the DNA Level for APO-A1 in Hepa1-6 Cells

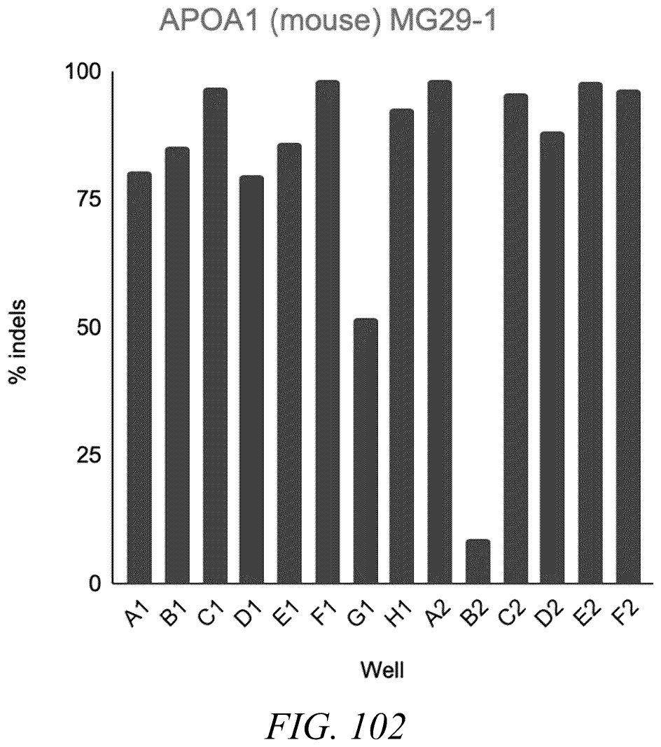

Nucleofection of MG29-1 RNPs (126 pmol protein/160 pmol guide) was performed into Hepa1-6 cells (200,000) using the Lonza 4D electroporator. Cells were harvested and genomic DNA prepared five days post-transfection. PCR primers appropriate for use in NGS-based DNA sequencing were generated, optimized, and used to amplify the individual target sequences for each guide RNA. The anmplicons were sequenced on an Illumina MiSeq machine and analyzed with a proprietary Python script to measure gene editing (FIG. 102).

TABLE 43A
Sequences of Guide RNAs and Sequences Targeted for Example 77

| | SEQ | | |
|---|---|---|---|
| | ID | | |
| Type | NO | Guide Name | SEQUENCE |
| MG29-1 sgRNA | 5847 | MG29-1-mouse | /AltR1/rUrArArUrUrUrCrUrArCrUrGrUrUrGrUrArGrArUrGr |
| targeting mouse | | APO-A1- | CrUrArArUrGrUrGrUrArUrGrUrGrGrArUrGrCrGrG/AltR2/ |
| APO-A1 | | sgRNA-A1 | |
| MG29-1 sgRNA | 5848 | MG29-1-mouse | /AltR1/rUrArArUrUrUrCrUrArCrUrGrUrUrGrUrArGrArUrAr |
| targeting mouse | | APO-A1- | ArUrCrCrUrCrCrUrCrCrUrUrGrGrGrCrCrArArCrA/AltR2/ |
| APO-A1 | | sgRNA-B1 | |
| MG29-1 sgRNA | 5849 | MG29-1-mouse | /AltR1/rUrArArUrUrUrCrUrArCrUrGrUrUrGrUrArGrArUrCr |
| targeting mouse | | APO-A1- | UrUrArCrUrUrCrArGrCrUrGrUrUrGrGrCrCrCrArA/AltR2/ |
| APO-A1 | | sgRNA-C1 | |
| MG29-1 sgRNA | 5850 | MG29-1-mouse | /AltR1/rUrArArUrUrUrCrUrArCrUrGrUrUrGrUrArGrArUrAr |
| targeting mouse | | APO-A1- | CrCrGrCrArUrCrCrArCrArUrArCrArCrArUrUrArG/AltR2/ |
| APO-A1 | | sgRNA-D1 | |
| MG29-1 sgRNA | 5851 | MG29-1-mouse | /AltR1/rUrArArUrUrUrCrUrArCrUrGrUrUrGrUrArGrArUrUr |
| targeting mouse | | APO-A1- | CrCrCrArUrUrGrGrGrArCrUrGrGrGrGrUrUrCrArU/AltR2/ |
| APO-A1 | | sgRNA-E1 | |
| MG29-1 sgRNA | 5852 | MG29-1-mouse | /AltR1/rUrArArUrUrUrCrUrArCrUrGrUrUrGrUrArGrArUrGr |
| targeting mouse | | APO-A1- | CrGrArCrCrGrCrArUrGrCrGrCrArCrArCrArCrGrU/AltR2/ |
| APO-A1 | | sgRNA-F1 | |
| MG29-1 sgRNA | 5853 | MG29-1-mouse | /AltR1/rUrArArUrUrUrCrUrArCrUrGrUrUrGrUrArGrArUrUr |
| targeting mouse | | APO-A1- | CrGrCrCrArArGrUrGrUrCrUrUrCrArGrGrUrGrGrG/AltR2/ |
| APO-A1 | | sgRNA-G1 | |
| MG29-1 sgRNA | 5854 | MG29-1-mouse | /AltR1/rUrArArUrUrUrCrUrArCrUrGrUrUrGrUrArGrArUrGr |
| targeting mouse | | APO-A1- | GrCrCrCrUrGrGrUrGrUrGrGrUrArCrUrCrGrUrUrC/AltR2/ |
| APO-A1 | | sgRNA-H1 | |
| MG29-1 sgRNA | 5855 | MG29-1-mouse | /AltR1/rUrArArUrUrUrCrUrArCrUrGrUrUrGrUrArGrArUrGr |
| targeting mouse | | APO-A1- | CrCrCrUrGrGrUrGrUrGrGrUrArCrUrCrGrUrUrCrA/AltR2/ |
| APO-A1 | | sgRNA-A2 | |
| MG29-1 sgRNA | 5856 | MG29-1-mouse | /AltR1/rUrArArUrUrUrCrUrArCrUrGrUrUrGrUrArGrArUrCr |
| targeting mouse | | APO-A1- | ArUrUrUrCrUrUrCrUrGrGrArArUrUrCrGrUrCrCrA/AltR2/ |
| APO-A1 | | sgRNA-B2 | |
| MG29-1 sgRNA | 5857 | MG29-1-mouse | /AltR1/rUrArArUrUrUrCrUrArCrUrGrUrUrGrUrArGrArUrUr |
| targeting mouse | | APO-A1- | UrCrUrGrGrArArUrUrCrGrUrCrCrArGrGrUrArGrG/AltR2/ |
| APO-A1 | | sgRNA-C2 | |
| MG29-1 sgRNA | 5858 | MG29-1-mouse | /AltR1/rUrArArUrUrUrCrUrArCrUrGrUrUrGrUrArGrArUrAr |
| targeting mouse | | APO-A1- | CrUrUrCrCrUrCrUrArGrGrUrCrCrUrUrGrUrUrCrA/AltR2/ |
| APO-A1 | | sgRNA-D2 | |
| MG29-1 sgRNA | 5859 | MG29-1-mouse | /AltR1/rUrArArUrUrUrCrUrArCrUrGrUrUrGrUrArGrArUrUr |
| targeting mouse | | APO-A1- | UrUrCrUrCrCrArGrGrUrUrArUrCrCrCrArGrArArG/AltR2/ |
| APO-A1 | | sgRNA-E2 | |
| MG29-1 sgRNA | 5860 | MG29-1-mouse | /AltR1/rUrArArUrUrUrCrUrArCrUrGrUrUrGrUrArGrArUrUr |
| targeting mouse | | APO-A1- | CrCrArGrGrUrUrArUrCrCrCrArGrArArGrUrCrCrC/AltR2/ |
| APO-A1 | | sgRNA-F2 | |
| DNA Sequence of | 5861 | MG29-1-mouse | GCTAATGTGTATGTGGATGCGG |
| APO-A1 Target Site | | APO-A1-target | |
| | | site-A1 | |
| DNA Sequence of | 5862 | MG29-1-mouse | AATCCTCCTCCTTGGGCCAACA |
| APO-A1 Target Site | | APO-A1-target | |
| | | site-B1 | |
| DNA Sequence of | 5863 | MG29-1-mouse | CTTACTTCAGCTGTTGGCCCAA |
| APO-A1 Target Site | | APO-A1-target | |
| | | site-C1 | |
| DNA Sequence of | 5864 | MG29-1-mouse | ACCGCATCCACATACACATTAG |
| APO-A1 Target Site | | APO-A1-target | |
| | | site-D1 | |
| DNA Sequence of | 5865 | MG29-1-mouse | TCCCATTGGGACTGGGGTTCAT |
| APO-A1 Target Site | | APO-A1-target | |
| | | site-E1 | |
| DNA Sequence of | 5866 | MG29-1-mouse | GCGACCGCATGCGCACACACGT |
| APO-A1 Target Site | | APO-A1-target | |
| | | site-F1 | |
| DNA Sequence of | 5867 | MG29-1-mouse | TCGCCAAGTGTCTTCAGGTGGG |
| APO-A1 Target Site | | APO-A1-target | |
| | | site-G1 | |
| DNA Sequence of | 5868 | MG29-1-mouse | GGCCCTGGTGTGGTACTCGTTC |
| APO-A1 Target Site | | APO-A1-target | |
| | | site-H1 | |
| DNA Sequence of | 5869 | MG29-1-mouse | GCCCTGGTGTGGTACTCGTTCA |
| APO-A1 Target Site | | APO-A1-target | |
| | | site-A2 | |
| DNA Sequence of | 5870 | MG29-1-mouse | CATTTCTTCTGGAATTCGTCCA |
| APO-A1 Target Site | | APO-A1-target | |
| | | site-B2 | |
| DNA Sequence of | 5871 | MG29-1-mouse | TTCTGGAATTCGTCCAGGTAGG |
| APO-A1 Target Site | | APO-A1-target | |
| | | site-C2 | |
| DNA Sequence of | 5872 | MG29-1-mouse | ACTTCCTCTAGGTCCTTGTTCA |
| APO-A1 Target Site | | APO-A1-target | |
| | | site-D2 | |
| DNA Sequence of | 5873 | MG29-1-mouse | TTTCTCCAGGTTATCCCAGAAG |
| APO-A1 Target Site | | APO-A1-target | |
| | | site-E2 | |
| DNA Sequence of | 5874 | MG29-1-mouse | TCCAGGTTATCCCAGAAGTCCC |
| APO-A1 Target Site | | APO-A1-target | |
| | | site-F2 | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 78—Gene editing outcomes at the DNA level for ANGPTL3 in Hepa1-6 Cells

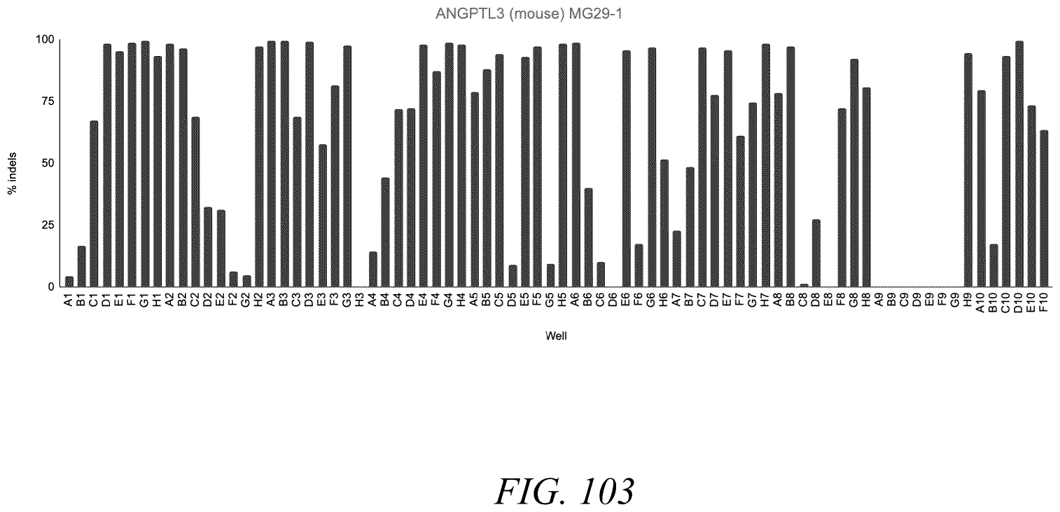

Nucleofection of MG29-1 RNPs (126 pmol protein/160 pmol guide) was performed into Hepa1-6 cells (200,000) using the Lonza 4D electroporator. Cells were harvested and genomic DNA prepared five days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 103).

TABLE 43B
Sequences of Guide RNAs and Sequences Targeted for Example 78

| | SEQ | | |
|---|---|---|---|
| | ID | | |
| Type | NO | Guide Name | SEQUENCE |
| MG29-1 | 5875 | MG29-1-mouse | /AltR1/rUrArArUrUrUrCrUrArCrUrGrUrUrGrUrArGrArUrUrGrUrUr |
| sgRNA | | ANGPTL3- | GrUrUrCrCrUrUrUrArGrUrArArUrUrGrCrA/AltR2/ |
| targeting | | sgRNA-A1 | |
| mouse | | | |
| ANGPTL3 | | | |
| MG29-1 | 5876 | MG29-1-mouse | /AltR1/rUrArArUrUrUrCrUrArCrUrGrUrUrGrUrArGrArUrGrUrUrGr |
| sgRNA | | ANGPTL3- | UrUrCrCrUrUrUrArGrUrArArUrUrGrCrArU/AltR2/ |
| targeting | | sgRNA-B1 | |
| mouse | | | |
| ANGPTL3 | | | |
| MG29-1 | 5877 | MG29-1-mouse | /AltR1/rUrArArUrUrUrCrUrArCrUrGrUrUrGrUrArGrArUrUrUrGrUr |
| sgRNA | | ANGPTL3- | UrCrCrUrUrUrArGrUrArArUrUrGrCrArUrC/AltR2/ |
| targeting | | sgRNA-C1 | |
| mouse | | | |
| ANGPTL3 | | | |
| MG29-1 | 5878 | MG29-1-mouse | /AltR1/rUrArArUrUrUrCrUrArCrUrGrUrUrGrUrArGrArUrGrUrArAr |
| sgRNA | | ANGPTL3- | UrUrGrCrArUrCrCrArGrArGrUrGrGrArUrC/AltR2/ |
| targeting | | sgRNA-D1 | |
| mouse | | | |
| ANGPTL3 | | | |
| MG29-1 | 5879 | MG29-1-mouse | /AltR1/rUrArArUrUrUrCrUrArCrUrGrUrUrGrUrArGrArUrArUrCrAr |
| sgRNA | | ANGPTL3- | UrUrUrGrArUrUrCrUrGrCrArCrCrUrUrCrA/AltR2/ |
| targeting | | sgRNA-E1 | |
| mouse | | | |
| ANGPTL3 | | | |
| MG29-1 | 5880 | MG29-1-mouse | /AltR1/rUrArArUrUrUrCrUrArCrUrGrUrUrGrUrArGrArUrArUrUrCr |
| sgRNA | | ANGPTL3- | UrGrCrArCrCrUrUrCrArGrArGrCrCrArArA/AltR2/ |
| targeting | | sgRNA-F1 | |
| mouse | | | |
| ANGPTL3 | | | |
| MG29-1 | 5881 | MG29-1-mouse | /AltR1/rUrArArUrUrUrCrUrArCrUrGrUrUrGrUrArGrArUrCrUrArUr |
| sgRNA | | ANGPTL3- | GrUrUrGrGrArUrGrArUrGrUrCrArArArArU/AltR2/ |
| targeting | | sgRNA-G1 | |
| mouse | | | |
| ANGPTL3 | | | |
| MG29-1 | 5882 | MG29-1-mouse | /AltR1/rUrArArUrUrUrCrUrArCrUrGrUrUrGrUrArGrArUrArGrCrGr |
| sgRNA | | ANGPTL3- | ArArUrGrGrCrCrUrCrCrUrGrCrArGrCrUrG/AltR2/ |
| targeting | | sgRNA-H1 | |
| mouse | | | |
| ANGPTL3 | | | |
| MG29-1 | 5883 | MG29-1-mouse | /AltR1/rUrArArUrUrUrCrUrArCrUrGrUrUrGrUrArGrArUrGrCrGrAr |
| sgRNA | | ANGPTL3- | ArUrGrGrCrCrUrCrCrUrGrCrArGrCrUrGrG/AltR2/ |
| targeting | | sgRNA-A2 | |
| mouse | | | |
| ANGPTL3 | | | |
| MG29-1 | 5884 | MG29-1-mouse | /AltR1/rUrArArUrUrUrCrUrArCrUrGrUrUrGrUrArGrArUrGrUrCrCr |
| sgRNA | | ANGPTL3- | ArUrArArGrArCrUrArArGrGrGrArCrArArA/AltR2/ |
| targeting | | sgRNA-B2 | |
| mouse | | | |
| ANGPTL3 | | | |
| MG29-1 | 5885 | MG29-1-mouse | /AltR1/rUrArArUrUrUrCrUrArCrUrGrUrUrGrUrArGrArUrUrCrCrAr |
| sgRNA | | ANGPTL3- | UrArArGrArCrUrArArGrGrGrArCrArArArU/AltR2/ |
| targeting | | sgRNA-C2 | |
| mouse | | | |
| ANGPTL3 | | | |
| MG29-1 | 5886 | MG29-1-mouse | /AltR1/rUrArArUrUrUrCrUrArCrUrGrUrUrGrUrArGrArUrArGrArAr |
| sgRNA | | ANGPTL3- | GrCrUrCrArArCrArUrArUrUrUrGrArUrCrA/AltR2/ |
| targeting | | sgRNA-D2 | |
| mouse | | | |
| ANGPTL3 | | | |
| MG29-1 | 5887 | MG29-1-mouse | /AltR1/rUrArArUrUrUrCrUrArCrUrGrUrUrGrUrArGrArUrArUrCrAr |
| sgRNA | | ANGPTL3- | GrUrCrUrUrUrUrUrArUrGrArCrCrUrArUrC/AltR2/ |
| targeting | | sgRNA-E2 | |
| mouse | | | |
| ANGPTL3 | | | |
| MG29-1 | 5888 | MG29-1-mouse | /AltR1/rUrArArUrUrUrCrUrArCrUrGrUrUrGrUrArGrArUrUrArUrGr |
| sgRNA | | ANGPTL3- | ArCrCrUrArUrCrArCrUrUrCrGrArArCrCrA/AltR2/ |
| targeting | | sgRNA-F2 | |
| mouse | | | |
| ANGPTL3 | | | |
| MG29-1 | 5889 | MG29-1-mouse | /AltR1/rUrArArUrUrUrCrUrArCrUrGrUrUrGrUrArGrArUrArUrGrAr |
| sgRNA | | ANGPTL3- | CrCrUrArUrCrArCrUrUrCrGrArArCrCrArA/AltR2/ |
| targeting | | sgRNA-G2 | |
| mouse | | | |
| ANGPTL3 | | | |
| MG29-1 | 5890 | MG29-1-mouse | /AltR1/rUrArArUrUrUrCrUrArCrUrGrUrUrGrUrArGrArUrUrGrArCr |
| sgRNA | | ANGPTL3- | CrUrArUrCrArCrUrUrCrGrArArCrCrArArU/AltR2/ |
| targeting | | sgRNA-H2 | |
| mouse | | | |
| ANGPTL3 | | | |
| MG29-1 | 5891 | MG29-1-mouse | /AltR1/rUrArArUrUrUrCrUrArCrUrGrUrUrGrUrArGrArUrGrArGrGr |
| sgRNA | | ANGPTL3- | ArGrCrArGrCrUrArArCrCrArArCrUrUrArA/AltR2/ |
| targeting | | sgRNA-A3 | |
| mouse | | | |
| ANGPTL3 | | | |
| MG29-1 | 5892 | MG29-1-mouse | /AltR1/rUrArArUrUrUrCrUrArCrUrGrUrUrGrUrArGrArUrUrArCrUr |
| sgRNA | | ANGPTL3- | UrArCrUrUrUrGrArGrUrGrArUrGrUrUrArC/AltR2/ |
| targeting | | sgRNA-B3 | |
| mouse | | | |
| ANGPTL3 | | | |
| MG29-1 | 5893 | MG29-1-mouse | /AltR1/rUrArArUrUrUrCrUrArCrUrGrUrUrGrUrArGrArUrArGrUrGr |
| sgRNA | | ANGPTL3- | ArUrGrUrUrArCrUrUrCrUrGrGrGrUrGrCrU/AltR2/ |
| targeting | | sgRNA-C3 | |
| mouse | | | |
| ANGPTL3 | | | |
| MG29-1 | 5894 | MG29-1-mouse | /AltR1/rUrArArUrUrUrCrUrArCrUrGrUrUrGrUrArGrArUrArGrUrUr |
| sgRNA | | ANGPTL3- | CrArGrUrUrCrUrArCrUrGrArCrArUrGrUrU/AltR2/ |
| targeting | | sgRNA-D3 | |
| mouse | | | |
| ANGPTL3 | | | |
| MG29-1 | 5895 | MG29-1-mouse | /AltR1/rUrArArUrUrUrCrUrArCrUrGrUrUrGrUrArGrArUrUrArArCr |
| sgRNA | | ANGPTL3- | UrUrGrUrArGrUrGrUrArGrArUrGrUrArGrU/AltR2/ |
| targeting | | sgRNA-E3 | |
| mouse | | | |
| ANGPTL3 | | | |
| MG29-1 | 5896 | MG29-1-mouse | /AltR1/rUrArArUrUrUrCrUrArCrUrGrUrUrGrUrArGrArUrArArCrUr |
| sgRNA | | ANGPTL3- | UrGrUrArGrUrGrUrArGrArUrGrUrArGrUrU/AltR2/ |
| targeting | | sgRNA-F3 | |
| mouse | | | |
| ANGPTL3 | | | |
| MG29-1 | 5897 | MG29-1-mouse | /AltR1/rUrArArUrUrUrCrUrArCrUrGrUrUrGrUrArGrArUrArCrUrUr |
| sgRNA | | ANGPTL3- | GrUrArGrUrGrUrArGrArUrGrUrArGrUrUrC/AltR2/ |
| targeting | | sgRNA-G3 | |
| mouse | | | |
| ANGPTL3 | | | |
| MG29-1 | 5898 | MG29-1-mouse | /AltR1/rUrArArUrUrUrCrUrArCrUrGrUrUrGrUrArGrArUrCrCrUrCr |
| sgRNA | | ANGPTL3- | UrUrCrUrUrUrGrArUrUrUrCrArUrUrGrGrU/AltR2/ |
| targeting | | sgRNA-H3 | |
| mouse | | | |
| ANGPTL3 | | | |
| MG29-1 | 5899 | MG29-1-mouse | /AltR1/rUrArArUrUrUrCrUrArCrUrGrUrUrGrUrArGrArUrCrUrCrUr |
| sgRNA | | ANGPTL3- | UrCrUrUrUrGrArUrUrUrCrArUrUrGrGrUrU/AltR2/ |
| targeting | | sgRNA-A4 | |
| mouse | | | |
| ANGPTL3 | | | |
| MG29-1 | 5900 | MG29-1-mouse | /AltR1/rUrArArUrUrUrCrUrArCrUrGrUrUrGrUrArGrArUrArUrUrUr |
| sgRNA | | ANGPTL3- | CrArUrUrGrGrUrUrCrGrArArGrUrGrArUrA/AltR2/ |
| targeting | | sgRNA-B4 | |
| mouse | | | |
| ANGPTL3 | | | |
| MG29-1 | 5901 | MG29-1-mouse | /AltR1/rUrArArUrUrUrCrUrArCrUrGrUrUrGrUrArGrArUrArUrUrGr |
| sgRNA | | ANGPTL3- | GrUrUrCrGrArArGrUrGrArUrArGrGrUrCrA/AltR2/ |
| targeting | | sgRNA-C4 | |
| mouse | | | |
| ANGPTL3 | | | |
| MG29-1 | 5902 | MG29-1-mouse | /AltR1/rUrArArUrUrUrCrUrArCrUrGrUrUrGrUrArGrArUrUrCrCrCr |
| sgRNA | | ANGPTL3- | UrUrArGrUrCrUrUrArUrGrGrArCrArArArA/AltR2/ |
| targeting | | sgRNA-D4 | |
| mouse | | | |
| ANGPTL3 | | | |
| MG29-1 | 5903 | MG29-1-mouse | /AltR1/rUrArArUrUrUrCrUrArCrUrGrUrUrGrUrArGrArUrArGrUrCr |
| sgRNA | | ANGPTL3- | CrArUrGrArCrCrCrArGrCrUrGrCrArGrGrA/AltR2/ |
| targeting | | sgRNA-E4 | |
| mouse | | | |
| ANGPTL3 | | | |
| MG29-1 | 5904 | MG29-1-mouse | /AltR1/rUrArArUrUrUrCrUrArCrUrGrUrUrGrUrArGrArUrGrArCrAr |
| sgRNA | | ANGPTL3- | UrCrArUrCrCrArArCrArUrArGrCrArArArU/AltR2/ |
| targeting | | sgRNA-F4 | |
| mouse | | | |
| ANGPTL3 | | | |
| MG29-1 | 5905 | MG29-1-mouse | /AltR1/rUrArArUrUrUrCrUrArCrUrGrUrUrGrUrArGrArUrArCrArUr |
| sgRNA | | ANGPTL3- | CrArUrCrCrArArCrArUrArGrCrArArArUrC/AltR2/ |
| targeting | | sgRNA-G4 | |
| mouse | | | |
| ANGPTL3 | | | |
| MG29-1 | 5906 | MG29-1-mouse | /AltR1/rUrArArUrUrUrCrUrArCrUrGrUrUrGrUrArGrArUrGrGrCrUr |
| sgRNA | | ANGPTL3- | CrUrGrArArGrGrUrGrCrArGrArArUrCrArA/AltR2/ |
| targeting | | sgRNA-H4 | |
| mouse | | | |
| ANGPTL3 | | | |
| MG29-1 | 5907 | MG29-1-mouse | /AltR1/rUrArArUrUrUrCrUrArCrUrGrUrUrGrUrArGrArUrGrCrUrCr |
| sgRNA | | ANGPTL3- | UrGrArArGrGrUrGrCrArGrArArUrCrArArA/AltR2/ |
| targeting | | sgRNA-A5 | |
| mouse | | | |
| ANGPTL3 | | | |
| MG29-1 | 5908 | MG29-1-mouse | /AltR1/rUrArArUrUrUrCrUrArCrUrGrUrUrGrUrArGrArUrGrUrArGr |
| sgRNA | | ANGPTL3- | ArArCrArGrCrArArGrArCrArArCrArGrCrA/AltR2/ |
| targeting | | sgRNA-B5 | |
| mouse | | | |
| ANGPTL3 | | | |
| MG29-1 | 5909 | MG29-1-mouse | /AltR1/rUrArArUrUrUrCrUrArCrUrGrUrUrGrUrArGrArUrUrArGrAr |
| sgRNA | | ANGPTL3- | ArCrArGrCrArArGrArCrArArCrArGrCrArU/AltR2/ |
| targeting | | sgRNA-C5 | |
| mouse | | | |
| ANGPTL3 | | | |
| MG29-1 | 5910 | MG29-1-mouse | /AltR1/rUrArArUrUrUrCrUrArCrUrGrUrUrGrUrArGrArUrCrUrArUr |
| sgRNA | | ANGPTL3- | UrUrCrUrUrUrUrArUrCrUrGrCrArUrGrUrG/AltR2/ |
| targeting | | sgRNA-D5 | |
| mouse | | | |
| ANGPTL3 | | | |
| MG29-1 | 5911 | MG29-1-mouse | /AltR1/rUrArArUrUrUrCrUrArCrUrGrUrUrGrUrArGrArUrUrArUrUr |
| sgRNA | | ANGPTL3- | UrCrUrUrUrUrArUrCrUrGrCrArUrGrUrGrC/AltR2/ |
| targeting | | sgRNA-E5 | |
| mouse | | | |
| ANGPTL3 | | | |
| MG29-1 | 5912 | MG29-1-mouse | /AltR1/rUrArArUrUrUrCrUrArCrUrGrUrUrGrUrArGrArUrUrUrUrUr |
| sgRNA | | ANGPTL3- | ArUrCrUrGrCrArUrGrUrGrCrUrGrUrUrGrA/AltR2/ |
| targeting | | sgRNA-F5 | |
| mouse | | | |
| ANGPTL3 | | | |
| MG29-1 | 5913 | MG29-1-mouse | /AltR1/rUrArArUrUrUrCrUrArCrUrGrUrUrGrUrArGrArUrArUrCrUr |
| sgRNA | | ANGPTL3- | GrCrArUrGrUrGrCrUrGrUrUrGrArCrUrUrA/AltR2/ |
| targeting | | sgRNA-G5 | |
| mouse | | | |
| ANGPTL3 | | | |
| MG29-1 | 5914 | MG29-1-mouse | /AltR1/rUrArArUrUrUrCrUrArCrUrGrUrUrGrUrArGrArUrUrCrUrGr |
| sgRNA | | ANGPTL3- | CrArUrGrUrGrCrUrGrUrUrGrArCrUrUrArA/AltR2/ |
| targeting | | sgRNA-H5 | |
| mouse | | | |
| ANGPTL3 | | | |
| MG29-1 | 5915 | MG29-1-mouse | /AltR1/rUrArArUrUrUrCrUrArCrUrGrUrUrGrUrArGrArUrUrArCrUr |
| sgRNA | | ANGPTL3- | GrUrUrCrUrUrCrCrArCrArCrUrCrUrGrGrA/AltR2/ |
| targeting | | sgRNA-A6 | |
| mouse | | | |
| ANGPTL3 | | | |
| MG29-1 | 5916 | MG29-1-mouse | /AltR1/rUrArArUrUrUrCrUrArCrUrGrUrUrGrUrArGrArUrUrArUrUr |
| sgRNA | | ANGPTL3- | CrUrUrUrUrArUrCrArGrCrUrCrArGrArArA/AltR2/ |
| targeting | | sgRNA-B6 | |
| mouse | | | |
| ANGPTL3 | | | |
| MG29-1 | 5917 | MG29-1-mouse | /AltR1/rUrArArUrUrUrCrUrArCrUrGrUrUrGrUrArGrArUrArUrCrAr |
| sgRNA | | ANGPTL3- | GrCrUrCrArGrArArArGrArCrUrGrGrUrArU/AltR2/ |
| targeting | | sgRNA-C6 | |
| mouse | | | |
| ANGPTL3 | | | |
| MG29-1 | 5918 | MG29-1-mouse | /AltR1/rUrArArUrUrUrCrUrArCrUrGrUrUrGrUrArGrArUrUrCrArGr |
| sgRNA | | ANGPTL3- | CrUrCrArGrArArArGrArCrUrGrGrUrArUrU/AltR2/ |
| targeting | | sgRNA-D6 | |
| mouse | | | |
| ANGPTL3 | | | |
| MG29-1 | 5919 | MG29-1-mouse | /AltR1/rUrArArUrUrUrCrUrArCrUrGrUrUrGrUrArGrArUrUrUrCrUr |
| sgRNA | | ANGPTL3- | ArArArUrCrArArGrArGrCrArCrCrArArGrA/AltR2/ |
| targeting | | sgRNA-E6 | |
| mouse | | | |
| ANGPTL3 | | | |
| MG29-1 | 5920 | MG29-1-mouse | /AltR1/rUrArArUrUrUrCrUrArCrUrGrUrUrGrUrArGrArUrCrUrGrUr |
| sgRNA | | ANGPTL3- | UrUrCrGrUrUrCrArGrUrUrGrArArGrArGrG/AltR2/ |
| targeting | | sgRNA-F6 | |
| mouse | | | |
| ANGPTL3 | | | |
| MG29-1 | 5921 | MG29-1-mouse | /AltR1/rUrArArUrUrUrCrUrArCrUrGrUrUrGrUrArGrArUrUrGrUrUr |
| sgRNA | | ANGPTL3- | UrCrGrUrUrCrArGrUrUrGrArArGrArGrGrG/AltR2/ |
| targeting | | sgRNA-G6 | |
| mouse | | | |
| ANGPTL3 | | | |
| MG29-1 | 5922 | MG29-1-mouse | /AltR1/rUrArArUrUrUrCrUrArCrUrGrUrUrGrUrArGrArUrGrUrUrCr |
| sgRNA | | ANGPTL3- | ArGrUrUrGrArArGrArGrGrGrGrGrArGrUrA/AltR2/ |
| targeting | | sgRNA-H6 | |
| mouse | | | |
| ANGPTL3 | | | |
| MG29-1 | 5923 | MG29-1-mouse | /AltR1/rUrArArUrUrUrCrUrArCrUrGrUrUrGrUrArGrArUrGrArArGr |
| sgRNA | | ANGPTL3- | ArArArGrArGrArArUrUrUrUrCrUrGrArGrG/AltR2/ |
| targeting | | sgRNA-A7 | |
| mouse | | | |
| ANGPTL3 | | | |
| MG29-1 | 5924 | MG29-1-mouse | /AltR1/rUrArArUrUrUrCrUrArCrUrGrUrUrGrUrArGrArUrCrUrGrAr |
| sgRNA | | ANGPTL3- | GrGrGrUrUrCrUrUrGrArArUrArCrCrArGrU/AltR2/ |
| targeting | | sgRNA-B7 | |
| mouse | | | |
| ANGPTL3 | | | |
| MG29-1 | 5925 | MG29-1-mouse | /AltR1/rUrArArUrUrUrCrUrArCrUrGrUrUrGrUrArGrArUrUrGrArGr |
| sgRNA | | ANGPTL3- | GrGrUrUrCrUrUrGrArArUrArCrCrArGrUrC/AltR2/ |
| targeting | | sgRNA-C7 | |
| mouse | | | |
| ANGPTL3 | | | |
| MG29-1 | 5926 | MG29-1-mouse | /AltR1/rUrArArUrUrUrCrUrArCrUrGrUrUrGrUrArGrArUrUrArArCr |
| sgRNA | | ANGPTL3- | ArGrArGrGrCrGrArArCrArUrArCrArArGrU/AltR2/ |
| targeting | | sgRNA-D7 | |
| mouse | | | |
| ANGPTL3 | | | |
| MG29-1 | 5927 | MG29-1-mouse | /AltR1/rUrArArUrUrUrCrUrArCrUrGrUrUrGrUrArGrArUrArUrGrUr |
| sgRNA | | ANGPTL3- | CrUrArCrUrGrUrGrArUrArCrCrCrArArUrC/AltR2/ |
| targeting | | sgRNA-E7 | |
| mouse | | | |
| ANGPTL3 | | | |
| MG29-1 | 5928 | MG29-1-mouse | /AltR1/rUrArArUrUrUrCrUrArCrUrGrUrUrGrUrArGrArUrCrArUrGr |
| sgRNA | | ANGPTL3- | GrGrUrUrUrArCrCrUrGrArUrUrGrGrGrUrA/AltR2/ |
| targeting | | sgRNA-F7 | |
| mouse | | | |
| ANGPTL3 | | | |
| MG29-1 | 5929 | MG29-1-mouse | /AltR1/rUrArArUrUrUrCrUrArCrUrGrUrUrGrUrArGrArUrCrCrUrGr |
| sgRNA | | ANGPTL3- | ArUrUrGrGrGrUrArUrCrArCrArGrUrArGrA/AltR2/ |
| targeting | | sgRNA-G7 | |
| mouse | | | |
| ANGPTL3 | | | |
| MG29-1 | 5930 | MG29-1-mouse | /AltR1/rUrArArUrUrUrCrUrArCrUrGrUrUrGrUrArGrArUrUrUrGrGr |
| sgRNA | | ANGPTL3- | UrUrUrArArUrArGrUrGrUrArCrArCrGrCrC/AltR2/ |
| targeting | | sgRNA-H7 | |
| mouse | | | |
| ANGPTL3 | | | |
| MG29-1 | 5931 | MG29-1-mouse | /AltR1/rUrArArUrUrUrCrUrArCrUrGrUrUrGrUrArGrArUrArUrArGr |
| sgRNA | | ANGPTL3- | UrGrUrArCrArCrGrCrCrArCrUrUrGrUrArU/AltR2/ |
| targeting | | sgRNA-A8 | |
| mouse | | | |
| ANGPTL3 | | | |
| MG29-1 | 5932 | MG29-1-mouse | /AltR1/rUrArArUrUrUrCrUrArCrUrGrUrUrGrUrArGrArUrCrCrArUr |
| sgRNA | | ANGPTL3- | CrGrArGrCrCrUrCrCrCrArArArGrCrCrCrU/AltR2/ |
| targeting | | sgRNA-B8 | |
| mouse | | | |
| ANGPTL3 | | | |
| MG29-1 | 5933 | MG29-1-mouse | /AltR1/rUrArArUrUrUrCrUrArCrUrGrUrUrGrUrArGrArUrCrGrUrAr |
| sgRNA | | ANGPTL3- | GrUrUrUrUrCrCrCrArUrGrUrUrUrCrGrUrU/AltR2/ |
| targeting | | sgRNA-C8 | |
| mouse | | | |
| ANGPTL3 | | | |
| MG29-1 | 5934 | MG29-1-mouse | /AltR1/rUrArArUrUrUrCrUrArCrUrGrUrUrGrUrArGrArUrGrUrArGr |
| sgRNA | | ANGPTL3- | UrUrUrUrCrCrCrArUrGrUrUrUrCrGrUrUrG/AltR2/ |
| targeting | | sgRNA-D8 | |
| mouse | | | |
| ANGPTL3 | | | |
| MG29-1 | 5935 | MG29-1-mouse | /AltR1/rUrArArUrUrUrCrUrArCrUrGrUrUrGrUrArGrArUrCrCrCrAr |
| sgRNA | | ANGPTL3- | UrGrUrUrUrCrGrUrUrGrArArGrUrCrCrUrG/AltR2/ |
| targeting | | sgRNA-E8 | |
| mouse | | | |
| ANGPTL3 | | | |
| MG29-1 | 5936 | MG29-1-mouse | /AltR1/rUrArArUrUrUrCrUrArCrUrGrUrUrGrUrArGrArUrCrCrArUr |
| sgRNA | | ANGPTL3- | GrUrUrUrCrGrUrUrGrArArGrUrCrCrUrGrU/AltR2/ |
| targeting | | sgRNA-F8 | |
| mouse | | | |
| ANGPTL3 | | | |
| MG29-1 | 5937 | MG29-1-mouse | /AltR1/rUrArArUrUrUrCrUrArCrUrGrUrUrGrUrArGrArUrGrUrUrGr |
| sgRNA | | ANGPTL3- | ArArGrUrCrCrUrGrUrGrArGrCrCrArUrCrU/AltR2/ |
| targeting | | sgRNA-G8 | |
| mouse | | | |
| ANGPTL3 | | | |
| MG29-1 | 5938 | MG29-1-mouse | /AltR1/rUrArArUrUrUrCrUrArCrUrGrUrUrGrUrArGrArUrCrGrGrUr |
| sgRNA | | ANGPTL3- | GrUrUrGrArArUrUrArArUrGrUrCrCrArUrG/AltR2/ |
| targeting | | sgRNA-H8 | |
| mouse | | | |
| ANGPTL3 | | | |
| MG29-1 | 5939 | MG29-1-mouse | /AltR1/rUrArArUrUrUrCrUrArCrUrGrUrUrGrUrArGrArUrCrCrCrUr |
| sgRNA | | ANGPTL3- | CrUrArArCrUrUrUrUrUrUrCrUrUrUrArGrG/AltR2/ |
| targeting | | sgRNA-A9 | |
| mouse | | | |
| ANGPTL3 | | | |
| MG29-1 | 5940 | MG29-1-mouse | /AltR1/rUrArArUrUrUrCrUrArCrUrGrUrUrGrUrArGrArUrCrCrUrCr |
| sgRNA | | ANGPTL3- | UrArArCrUrUrUrUrUrUrCrUrUrUrArGrGrA/AltR2/ |
| targeting | | sgRNA-B9 | |
| mouse | | | |
| ANGPTL3 | | | |
| MG29-1 | 5941 | MG29-1-mouse | /AltR1/rUrArArUrUrUrCrUrArCrUrGrUrUrGrUrArGrArUrUrUrCrUr |
| sgRNA | | ANGPTL3- | UrUrArGrGrArGrArArUrUrUrUrGrGrUrUrG/AltR2/ |
| targeting | | sgRNA-C9 | |
| mouse | | | |
| ANGPTL3 | | | |
| MG29-1 | 5942 | MG29-1-mouse | /AltR1/rUrArArUrUrUrCrUrArCrUrGrUrUrGrUrArGrArUrUrCrUrUr |
| sgRNA | | ANGPTL3- | UrArGrGrArGrArArUrUrUrUrGrGrUrUrGrG/AltR2/ |
| targeting | | sgRNA-D9 | |
| mouse | | | |
| ANGPTL3 | | | |
| MG29-1 | 5943 | MG29-1-mouse | /AltR1/rUrArArUrUrUrCrUrArCrUrGrUrUrGrUrArGrArUrCrUrUrUr |
| sgRNA | | ANGPTL3- | ArGrGrArGrArArUrUrUrUrGrGrUrUrGrGrG/AltR2/ |
| targeting | | sgRNA-E9 | |
| mouse | | | |
| ANGPTL3 | | | |
| MG29-1 | 5944 | MG29-1-mouse | /AltR1/rUrArArUrUrUrCrUrArCrUrGrUrUrGrUrArGrArUrUrUrUrAr |
| sgRNA | | ANGPTL3- | GrGrArGrArArUrUrUrUrGrGrUrUrGrGrGrC/AltR2/ |
| targeting | | sgRNA-F9 | |
| mouse | | | |
| ANGPTL3 | | | |
| MG29-1 | 5945 | MG29-1-mouse | /AltR1/rUrArArUrUrUrCrUrArCrUrGrUrUrGrUrArGrArUrGrGrArGr |
| sgRNA | | ANGPTL3- | ArArUrUrUrUrGrGrUrUrGrGrGrCrCrUrArG/AltR2/ |
| targeting | | sgRNA-G9 | |
| mouse | | | |
| ANGPTL3 | | | |
| MG29-1 | 5946 | MG29-1-mouse | /AltR1/rUrArArUrUrUrCrUrArCrUrGrUrUrGrUrArGrArUrGrGrUrUr |
| sgRNA | | ANGPTL3- | GrGrGrCrCrUrArGrArGrArArGrArUrCrUrA/AltR2/ |
| targeting | | sgRNA-H9 | |
| mouse | | | |
| ANGPTL3 | | | |
| MG29-1 | 5947 | MG29-1-mouse | /AltR1/rUrArArUrUrUrCrUrArCrUrGrUrUrGrUrArGrArUrGrUrUrGr |
| sgRNA | | ANGPTL3- | GrGrCrCrUrArGrArGrArArGrArUrCrUrArU/AltR2/ |
| targeting | | sgRNA-A10 | |
| mouse | | | |
| ANGPTL3 | | | |
| MG29-1 | 5948 | MG29-1-mouse | /AltR1/rUrArArUrUrUrCrUrArCrUrGrUrUrGrUrArGrArUrArCrGrAr |
| sgRNA | | ANGPTL3- | CrUrCrGrArGrCrUrArCrArArGrArCrUrGrG/AltR2/ |
| targeting | | sgRNA-B10 | |
| mouse | | | |
| ANGPTL3 | | | |
| MG29-1 | 5949 | MG29-1-mouse | /AltR1/rUrArArUrUrUrCrUrArCrUrGrUrUrGrUrArGrArUrCrGrArCr |
| sgRNA | | ANGPTL3- | UrCrGrArGrCrUrArCrArArGrArCrUrGrGrA/AltR2/ |
| targeting | | sgRNA-C10 | |
| mouse | | | |
| ANGPTL3 | | | |
| MG29-1 | 5950 | MG29-1-mouse | /AltR1/rUrArArUrUrUrCrUrArCrUrGrUrUrGrUrArGrArUrArCrCrUr |
| sgRNA | | ANGPTL3- | GrGrGrCrArGrUrCrArCrGrArArArCrCrArA/AltR2/ |
| targeting | | sgRNA-D10 | |
| mouse | | | |
| ANGPTL3 | | | |
| MG29-1 | 5951 | MG29-1-mouse | /AltR1/rUrArArUrUrUrCrUrArCrUrGrUrUrGrUrArGrArUrGrUrGrAr |
| sgRNA | | ANGPTL3- | CrUrGrCrCrCrArGrGrUrGrArArArGrGrArG/AltR2/ |
| targeting | | sgRNA-E10 | |
| mouse | | | |
| ANGPTL3 | | | |
| MG29-1 | 5952 | MG29-1-mouse | /AltR1/rUrArArUrUrUrCrUrArCrUrGrUrUrGrUrArGrArUrCrArGrUr |
| sgRNA | | ANGPTL3- | CrUrUrGrUrArGrCrUrCrGrArGrUrCrGrUrA/AltR2/ |
| targeting | | sgRNA-F10 | |
| mouse | | | |
| ANGPTL3 | | | |
| DNA | 5953 | MG29-1-mouse | TGTTGTTCCTTTAGTAATTGCA |
| Sequence | | ANGPTL3- | |
| of | | target site-A1 | |
| ANGPTL3 | | | |
| Target Site | | | |
| DNA | 5954 | MG29-1-mouse | GTTGTTCCTTTAGTAATTGCAT |
| Sequence | | ANGPTL3- | |
| of | | target site-B1 | |
| ANGPTL3 | | | |
| Target Site | | | |
| DNA | 5955 | MG29-1-mouse | TTGTTCCTTTAGTAATTGCATC |
| Sequence | | ANGPTL3- | |
| of | | target site-C1 | |
| ANGPTL3 | | | |
| Target Site | | | |
| DNA | 5956 | MG29-1-mouse | GTAATTGCATCCAGAGTGGATC |
| Sequence | | ANGPTL3- | |
| of | | target site-D1 | |
| ANGPTL3 | | | |
| Target Site | | | |
| DNA | 5957 | MG29-1-mouse | ATCATTTGATTCTGCACCTTCA |
| Sequence | | ANGPTL3- | |
| of | | target site-E1 | |
| ANGPTL3 | | | |
| Target Site | | | |
| DNA | 5958 | MG29-1-mouse | ATTCTGCACCTTCAGAGCCAAA |
| Sequence | | ANGPTL3- | |
| of | | target site-F1 | |
| ANGPTL3 | | | |
| Target Site | | | |
| DNA | 5959 | MG29-1-mouse | CTATGTTGGATGATGTCAAAAT |
| Sequence | | ANGPTL3- | |
| of | | target site-G1 | |
| ANGPTL3 | | | |
| Target Site | | | |
| DNA | 5960 | MG29-1-mouse | AGCGAATGGCCTCCTGCAGCTG |
| Sequence | | ANGPTL3- | |
| of | | target site-H1 | |
| ANGPTL3 | | | |
| Target Site | | | |
| DNA | 5961 | MG29-1-mouse | GCGAATGGCCTCCTGCAGCTGG |
| Sequence | | ANGPTL3- | |
| of | | target site-A2 | |
| ANGPTL3 | | | |
| Target Site | | | |
| DNA | 5962 | MG29-1-mouse | GTCCATAAGACTAAGGGACAAA |
| Sequence | | ANGPTL3- | |
| of | | target site-B2 | |
| ANGPTL3 | | | |
| Target Site | | | |
| DNA | 5963 | MG29-1-mouse | TCCATAAGACTAAGGGACAAAT |
| Sequence | | ANGPTL3- | |
| of | | target site-C2 | |
| ANGPTL3 | | | |
| Target Site | | | |
| DNA | 5964 | MG29-1-mouse | AGAAGCTCAACATATTTGATCA |
| Sequence | | ANGPTL3- | |
| of | | target site-D2 | |
| ANGPTL3 | | | |
| Target Site | | | |
| DNA | 5965 | MG29-1-mouse | ATCAGTCTTTTTATGACCTATC |
| Sequence | | ANGPTL3- | |
| of | | target site-E2 | |
| ANGPTL3 | | | |
| Target Site | | | |
| DNA | 5966 | MG29-1-mouse | TATGACCTATCACTTCGAACCA |
| Sequence | | ANGPTL3- | |
| of | | target site-F2 | |
| ANGPTL3 | | | |
| Target Site | | | |
| DNA | 5967 | MG29-1-mouse | ATGACCTATCACTTCGAACCAA |
| Sequence | | ANGPTL3- | |
| of | | target site-G2 | |
| ANGPTL3 | | | |
| Target Site | | | |
| DNA | 5968 | MG29-1-mouse | TGACCTATCACTTCGAACCAAT |
| Sequence | | ANGPTL3- | |
| of | | target site-H2 | |
| ANGPTL3 | | | |
| Target Site | | | |
| DNA | 5969 | MG29-1-mouse | GAGGAGCAGCTAACCAACTTAA |
| Sequence | | ANGPTL3- | |
| of | | target site-A3 | |
| ANGPTL3 | | | |
| Target Site | | | |
| DNA | 5970 | MG29-1-mouse | TACTTACTTTGAGTGATGTTAC |
| Sequence | | ANGPTL3- | |
| of | | target site-B3 | |
| ANGPTL3 | | | |
| Target Site | | | |
| DNA | 5971 | MG29-1-mouse | AGTGATGTTACTTCTGGGTGCT |
| Sequence | | ANGPTL3- | |
| of | | target site-C3 | |
| ANGPTL3 | | | |
| Target Site | | | |
| DNA | 5972 | MG29-1-mouse | AGTTCAGTTCTACTGACATGTT |
| Sequence | | ANGPTL3- | |
| of | | target site-D3 | |
| ANGPTL3 | | | |
| Target Site | | | |
| DNA | 5973 | MG29-1-mouse | TAACTTGTAGTGTAGATGTAGT |
| Sequence | | ANGPTL3- | |
| of | | target site-E3 | |
| ANGPTL3 | | | |
| Target Site | | | |
| DNA | 5974 | MG29-1-mouse | AACTTGTAGTGTAGATGTAGTT |
| Sequence | | ANGPTL3- | |
| of | | target site-F3 | |
| ANGPTL3 | | | |
| Target Site | | | |
| DNA | 5975 | MG29-1-mouse | ACTTGTAGTGTAGATGTAGTTC |
| Sequence | | ANGPTL3- | |
| of | | target site-G3 | |
| ANGPTL3 | | | |
| Target Site | | | |
| DNA | 5976 | MG29-1-mouse | CCTCTTCTTTGATTTCATTGGT |
| Sequence | | ANGPTL3- | |
| of | | target site-H3 | |
| ANGPTL3 | | | |
| Target Site | | | |
| DNA | 5977 | MG29-1-mouse | CTCTTCTTTGATTTCATTGGTT |
| Sequence | | ANGPTL3- | |
| of | | target site-A4 | |
| ANGPTL3 | | | |
| Target Site | | | |
| DNA | 5978 | MG29-1-mouse | ATTTCATTGGTTCGAAGTGATA |
| Sequence | | ANGPTL3- | |
| of | | target site-B4 | |
| ANGPTL3 | | | |
| Target Site | | | |
| DNA | 5979 | MG29-1-mouse | ATTGGTTCGAAGTGATAGGTCA |
| Sequence | | ANGPTL3- | |
| of | | target site-C4 | |
| ANGPTL3 | | | |
| Target Site | | | |
| DNA | 5980 | MG29-1-mouse | TCCCTTAGTCTTATGGACAAAA |
| Sequence | | ANGPTL3- | |
| of | | target site-D4 | |
| ANGPTL3 | | | |
| Target Site | | | |
| DNA | 5981 | MG29-1-mouse | AGTCCATGACCCAGCTGCAGGA |
| Sequence | | ANGPTL3- | |
| of | | target site-E4 | |
| ANGPTL3 | | | |
| Target Site | | | |
| DNA | 5982 | MG29-1-mouse | GACATCATCCAACATAGCAAAT |
| Sequence | | ANGPTL3- | |
| of | | target site-F4 | |
| ANGPTL3 | | | |
| Target Site | | | |
| DNA | 5983 | MG29-1-mouse | ACATCATCCAACATAGCAAATC |
| Sequence | | ANGPTL3- | |
| of | | target site-G4 | |
| ANGPTL3 | | | |
| Target Site | | | |
| DNA | 5984 | MG29-1-mouse | GGCTCTGAAGGTGCAGAATCAA |
| Sequence | | ANGPTL3- | |
| of | | target site-H4 | |
| ANGPTL3 | | | |
| Target Site | | | |
| DNA | 5985 | MG29-1-mouse | GCTCTGAAGGTGCAGAATCAAA |
| Sequence | | ANGPTL3- | |
| of | | target site-A5 | |
| ANGPTL3 | | | |
| Target Site | | | |
| DNA | 5986 | MG29-1-mouse | GTAGAACAGCAAGACAACAGCA |
| Sequence | | ANGPTL3- | |
| of | | target site-B5 | |
| ANGPTL3 | | | |
| Target Site | | | |
| DNA | 5987 | MG29-1-mouse | TAGAACAGCAAGACAACAGCAT |
| Sequence | | ANGPTL3- | |
| of | | target site-C5 | |
| ANGPTL3 | | | |
| Target Site | | | |
| DNA | 5988 | MG29-1-mouse | CTATTTCTTTTATCTGCATGTG |
| Sequence | | ANGPTL3- | |
| of | | target site-D5 | |
| ANGPTL3 | | | |
| Target Site | | | |
| DNA | 5989 | MG29-1-mouse | TATTTCTTTTATCTGCATGTGC |
| Sequence | | ANGPTL3- | |
| of | | target site-E5 | |
| ANGPTL3 | | | |
| Target Site | | | |
| DNA | 5990 | MG29-1-mouse | TTTTATCTGCATGTGCTGTTGA |
| Sequence | | ANGPTL3- | |
| of | | target site-F5 | |
| ANGPTL3 | | | |
| Target Site | | | |
| DNA | 5991 | MG29-1-mouse | ATCTGCATGTGCTGTTGACTTA |
| Sequence | | ANGPTL3- | |
| of | | target site-G5 | |
| ANGPTL3 | | | |
| Target Site | | | |
| DNA | 5992 | MG29-1-mouse | TCTGCATGTGCTGTTGACTTAA |
| Sequence | | ANGPTL3- | |
| of | | target site-H5 | |
| ANGPTL3 | | | |
| Target Site | | | |
| DNA | 5993 | MG29-1-mouse | TACTGTTCTTCCACACTCTGGA |
| Sequence | | ANGPTL3- | |
| of | | target site-A6 | |
| ANGPTL3 | | | |
| Target Site | | | |
| DNA | 5994 | MG29-1-mouse | TATTCTTTTATCAGCTCAGAAA |
| Sequence | | ANGPTL3- | |
| of | | target site-B6 | |
| ANGPTL3 | | | |
| Target Site | | | |
| DNA | 5995 | MG29-1-mouse | ATCAGCTCAGAAAGACTGGTAT |
| Sequence | | ANGPTL3- | |
| of | | target site-C6 | |
| ANGPTL3 | | | |
| Target Site | | | |
| DNA | 5996 | MG29-1-mouse | TCAGCTCAGAAAGACTGGTATT |
| Sequence | | ANGPTL3- | |
| of | | target site-D6 | |
| ANGPTL3 | | | |
| Target Site | | | |
| DNA | 5997 | MG29-1-mouse | TTCTAAATCAAGAGCACCAAGA |
| Sequence | | ANGPTL3- | |
| of | | target site-E6 | |
| ANGPTL3 | | | |
| Target Site | | | |
| DNA | 5998 | MG29-1-mouse | CTGTTTCGTTCAGTTGAAGAGG |
| Sequence | | ANGPTL3- | |
| of | | target site-F6 | |
| ANGPTL3 | | | |
| Target Site | | | |
| DNA | 5999 | MG29-1-mouse | TGTTTCGTTCAGTTGAAGAGGG |
| Sequence | | ANGPTL3- | |
| of | | target site-G6 | |
| ANGPTL3 | | | |
| Target Site | | | |
| DNA | 6000 | MG29-1-mouse | GTTCAGTTGAAGAGGGGGAGTA |
| Sequence | | ANGPTL3- | |
| of | | target site-H6 | |
| ANGPTL3 | | | |
| Target Site | | | |
| DNA | 6001 | MG29-1-mouse | GAAGAAAGAGAATTTTCTGAGG |
| Sequence | | ANGPTL3- | |
| of | | target site-A7 | |
| ANGPTL3 | | | |
| Target Site | | | |
| DNA | 6002 | MG29-1-mouse | CTGAGGGTTCTTGAATACCAGT |
| Sequence | | ANGPTL3- | |
| of | | target site-B7 | |
| ANGPTL3 | | | |
| Target Site | | | |
| DNA | 6003 | MG29-1-mouse | TGAGGGTTCTTGAATACCAGTC |
| Sequence | | ANGPTL3- | |
| of | | target site-C7 | |
| ANGPTL3 | | | |
| Target Site | | | |
| DNA | 6004 | MG29-1-mouse | TAACAGAGGCGAACATACAAGT |
| Sequence | | ANGPTL3- | |
| of | | target site-D7 | |
| ANGPTL3 | | | |
| Target Site | | | |
| DNA | 6005 | MG29-1-mouse | ATGTCTACTGTGATACCCAATC |
| Sequence | | ANGPTL3- | |
| of | | target site-E7 | |
| ANGPTL3 | | | |
| Target Site | | | |
| DNA | 6006 | MG29-1-mouse | CATGGGTTTACCTGATTGGGTA |
| Sequence | | ANGPTL3- | |
| of | | target site-F7 | |
| ANGPTL3 | | | |
| Target Site | | | |
| DNA | 6007 | MG29-1-mouse | CCTGATTGGGTATCACAGTAGA |
| Sequence | | ANGPTL3- | |
| of | | target site-G7 | |
| ANGPTL3 | | | |
| Target Site | | | |
| DNA | 6008 | MG29-1-mouse | TTGGTTTAATAGTGTACACGCC |
| Sequence | | ANGPTL3- | |
| of | | target site-H7 | |
| ANGPTL3 | | | |
| Target Site | | | |
| DNA | 6009 | MG29-1-mouse | ATAGTGTACACGCCACTTGTAT |
| Sequence | | ANGPTL3- | |
| of | | target site-A8 | |
| ANGPTL3 | | | |
| Target Site | | | |
| DNA | 6010 | MG29-1-mouse | CCATCGAGCCTCCCAAAGCCCT |
| Sequence | | ANGPTL3- | |
| of | | target site-B8 | |
| ANGPTL3 | | | |
| Target Site | | | |
| DNA | 6011 | MG29-1-mouse | CGTAGTTTTCCCATGTTTCGTT |
| Sequence | | ANGPTL3- | |
| of | | target site-C8 | |
| ANGPTL3 | | | |
| Target Site | | | |
| DNA | 6012 | MG29-1-mouse | GTAGTTTTCCCATGTTTCGTTG |
| Sequence | | ANGPTL3- | |
| of | | target site-D8 | |
| ANGPTL3 | | | |
| Target Site | | | |
| DNA | 6013 | MG29-1-mouse | CCCATGTTTCGTTGAAGTCCTG |
| Sequence | | ANGPTL3- | |
| of | | target site-E8 | |
| ANGPTL3 | | | |
| Target Site | | | |
| DNA | 6014 | MG29-1-mouse | CCATGTTTCGTTGAAGTCCTGT |
| Sequence | | ANGPTL3- | |
| of | | target site-F8 | |
| ANGPTL3 | | | |
| Target Site | | | |
| DNA | 6015 | MG29-1-mouse | GTTGAAGTCCTGTGAGCCATCT |
| Sequence | | ANGPTL3- | |
| of | | target site-G8 | |
| ANGPTL3 | | | |
| Target Site | | | |
| DNA | 6016 | MG29-1-mouse | CGGTGTTGAATTAATGTCCATG |
| Sequence | | ANGPTL3- | |
| of | | target site-H8 | |
| ANGPTL3 | | | |
| Target Site | | | |
| DNA | 6017 | MG29-1-mouse | CCCTCTAACTTTTTTCTTTAGG |
| Sequence | | ANGPTL3- | |
| of | | target site-A9 | |
| ANGPTL3 | | | |
| Target Site | | | |
| DNA | 6018 | MG29-1-mouse | CCTCTAACTTTTTTCTTTAGGA |
| Sequence | | ANGPTL3- | |
| of | | target site-B9 | |
| ANGPTL3 | | | |
| Target Site | | | |
| DNA | 6019 | MG29-1-mouse | TTCTTTAGGAGAATTTTGGTTG |
| Sequence | | ANGPTL3- | |
| of | | target site-C9 | |
| ANGPTL3 | | | |
| Target Site | | | |
| DNA | 6020 | MG29-1-mouse | TCTTTAGGAGAATTTTGGTTGG |
| Sequence | | ANGPTL3- | |
| of | | target site-D9 | |
| ANGPTL3 | | | |
| Target Site | | | |
| DNA | 6021 | MG29-1-mouse | CTTTAGGAGAATTTTGGTTGGG |
| Sequence | | ANGPTL3- | |
| of | | target site-E9 | |
| ANGPTL3 | | | |
| Target Site | | | |
| DNA | 6022 | MG29-1-mouse | TTTAGGAGAATTTTGGTTGGGC |
| Sequence | | ANGPTL3- | |
| of | | target site-F9 | |
| ANGPTL3 | | | |
| Target Site | | | |
| DNA | 6023 | MG29-1-mouse | GGAGAATTTTGGTTGGGCCTAG |
| Sequence | | ANGPTL3- | |
| of | | target site-G9 | |
| ANGPTL3 | | | |
| Target Site | | | |
| DNA | 6024 | MG29-1-mouse | GGTTGGGCCTAGAGAAGATCTA |
| Sequence | | ANGPTL3- | |
| of | | target site-H9 | |
| ANGPTL3 | | | |
| Target Site | | | |
| DNA | 6025 | MG29-1-mouse | GTTGGGCCTAGAGAAGATCTAT |
| Sequence | | ANGPTL3- | |
| of | | target site-A10 | |
| ANGPTL3 | | | |
| Target Site | | | |
| DNA | 6026 | MG29-1-mouse | ACGACTCGAGCTACAAGACTGG |
| Sequence | | ANGPTL3- | |
| of | | target site-B10 | |
| ANGPTL3 | | | |
| Target Site | | | |
| DNA | 6027 | MG29-1-mouse | CGACTCGAGCTACAAGACTGGA |
| Sequence | | ANGPTL3- | |
| of | | target site-C10 | |
| ANGPTL3 | | | |
| Target Site | | | |
| DNA | 6028 | MG29-1-mouse | ACCTGGGCAGTCACGAAACCAA |
| Sequence | | ANGPTL3- | |
| of | | target site-D10 | |
| ANGPTL3 | | | |
| Target Site | | | |
| DNA | 6029 | MG29-1-mouse | GTGACTGCCCAGGTGAAAGGAG |
| Sequence | | ANGPTL3- | T |
| of | | target site-E10 | |
| ANGPTL3 | | | |
| Target Site | | | |
| DNA | 6030 | MG29-1-mouse | CAGTCTTGTAGCTCGAGTCGTA |
| Sequence | | ANGPTL3- | |
| of | | target site-F10 | |
| ANGPTL3 | | | |
| Target Site | | | |

r; native RNA linkage comprising the sugar ribose (for example the ribose or RNA form of the A base is written rA), d; deoxyribose sugar (DNA) linkage (for example a deoxyribose form of the A base is written dA), *; between bases in which one of the oxygen molecules in the phosphodiester bond is replaced with sulfur; AltR1 and AltR2 refer to IDT technologies' proprietary 5′ and 3′ AltR modifications

### Example 79—Compact MG55-43 Nuclease System

In Vitro Characterization to Identify Putative tracrRNAs

To identify tracrRNA sequences, the nuclease (MG55-43, protein SEQ ID NO: 470), intergenic sequences, and minimal arrays were expressed in transcription-translation reaction mixtures using myTXTL® Sigma 70 Master Mix Kit (Arbor Biosciences). The final reaction mixtures contained 5 nM nuclease DNA template, 12 nM intergenic DNA template, 15 nM minimal array DNA template, 0.1 nM pTXTL-P70a-T7map, and 1× of myTXTL® Sigma 70 Master Mix. The reactions were incubated at 29° C. for 16 hours then stored at 4° C.

Ribonucleoprotein complexes were tested via in vitro cleavage reactions. Plasmid DNA library cleavage reactions were carried out by mixing 5 nM of the target plasmid DNA library representing all possible 8N PAMs, a 5-fold dilution of the TXTL expressions, 10 nM Tris-HCl, 10 nM MgCl₂, and 100 mM NaCl at 37° C. for 2 hours. Reactions were stopped and cleaned with HighPrep™ PCR clean up beads (MAGBIO Genomics, Inc.) and eluted in Tris EDTA pH 8.0 buffer. 3 nM of the cleavage product ends were blunted with 3.33 μM dNTPs, 1× T4 DNA ligase buffer, and 0.167 U/μL of Klenow Fragment (New England Biolabs Inc.) at 25° C. for 15 minutes. 1.5 nM of the cleavage products were ligated with 150 nM adapters, 1× T4 DNA ligase buffer (New England Biolabs Inc.), and 20 U/μL T4 DNA ligase (New England Biolabs Inc.) at room temperature for 20 minutes. The ligated products were amplified by PCR with NGS primers and sequenced by NGS to obtain the PAM.

To obtain the sequence of the tracrRNA and crRNA, RNA is extracted from TXTL lysate following the Quick-RNA™ Miniprep Kit (Zymo Research) and eluted in 30-50 μL of water. The total concentration of the transcripts was measured on a Nanodrop, Tapestation, and Qubit. RNA libraries were subjected to RNA sequencing.

In Silico Search for Novel tracrRNA Sequences

To identify additional non-coding regions containing potential tracrRNAs, the sequence of the active tracrRNA was mapped to other contigs containing nucleases in the same nuclease family. The newly identified sequences were used to generate covariance models to predict additional tracrRNAs. Covariance models were built from a multiple sequence alignment (MSA) of the active and predicted tracrRNA sequences. The secondary structure of the MSA was obtained with RNAalifold (Vienna Package), and the covariance models were built with Infernal packages (http://eddylab.org/infernal/). Contigs containing candidate nucleases were searched using the covariance models with the Infernal command ‘cmsearch’.

sgRNA Design

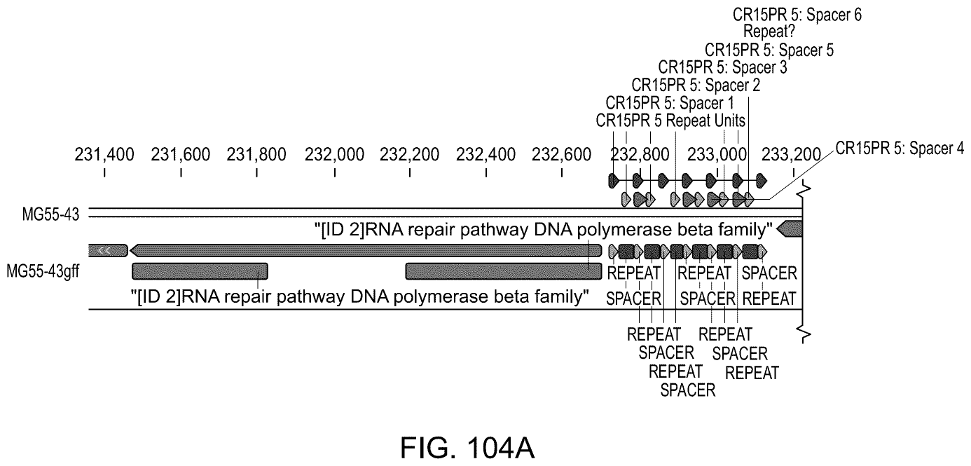

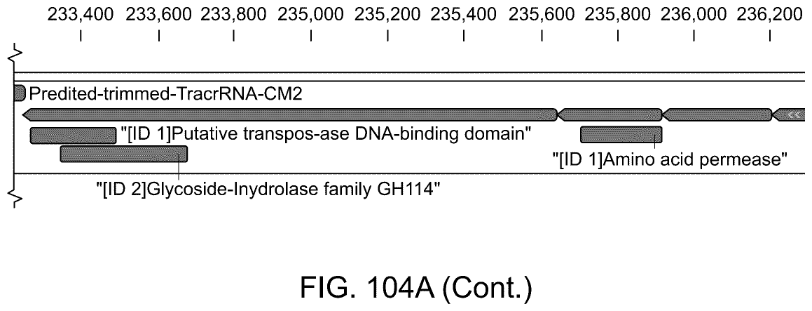

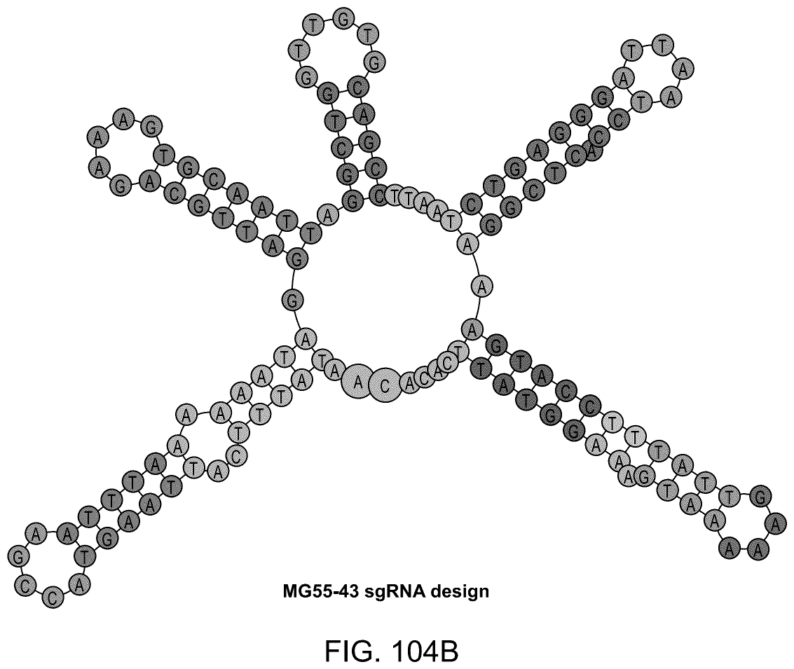

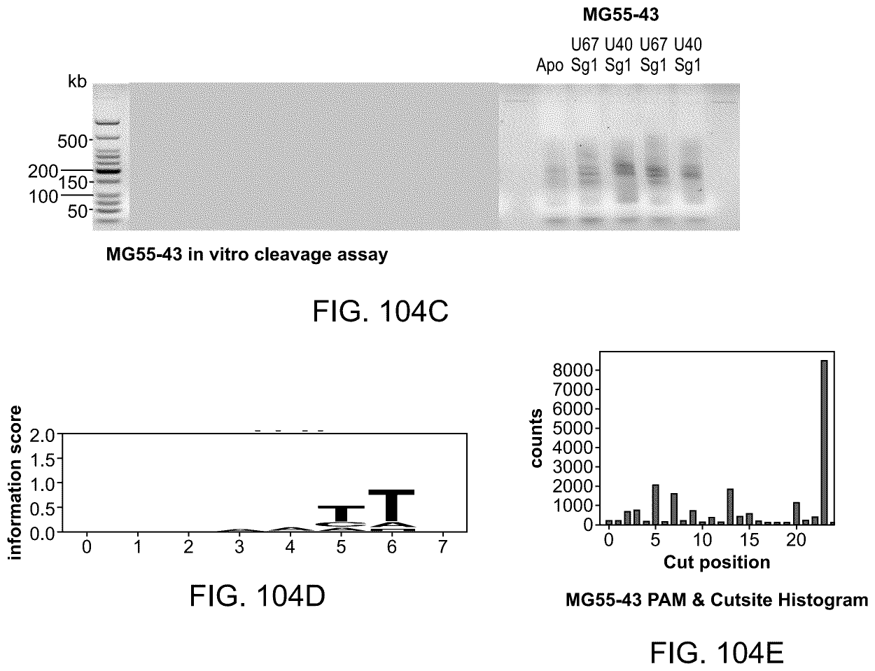

The predicted tracrRNA obtained from the covariance models and associated CRISPR repeat sequence were modified to generate an sgRNA (FIG. 104A, SEQ ID NO: 6031) as follows: the 3′ end of the predicted tracrRNA sequence as well as the 5′ end of the repeat sequence were trimmed, and then connected with a GAAA tetraloop (FIG. 104B).

### In Vitro Cleavage Reactions Confirmed MG55-43 Activity and Enabled PAM Determination

Target plasmid DNA library cleavage reactions described above (In vitro characterization to Identify putative tracrRNAs) were carried out using the guides. The product of the reactions were amplified by PCR with NGS primers and sequenced by NGS to obtain the PAM. Active proteins that successfully cleaved the PAM library yielded a band around 188 or 205 bp in agarose gel electrophoresis (FIG. 104C).

TABLE 44
Spacer sequences for tested guides

| Code | Sequence |
|---|---|
| U67 spacer | GTCGAGGCTTGCGACGTGGT |
| U40 spacer | TGGAGATATCTTGAACCTTG |

Sequence logos for PAMs were made using Seqlogo maker, and histograms showing cut-site preferences were made from the counts of reads mapping at each nucleotide position. The PAM recognized by MG55-43 is a 5′-yTn sequence (FIG. 104D, Sequence Number: A6032), and the preferred cut position is nucleotide 23 (FIG. 104E).

### Example 80—MG91 Family of Compact Type V Nucleases

De Novo Prediction of tracrRNA Sequences Encoded in Intergenic Regions

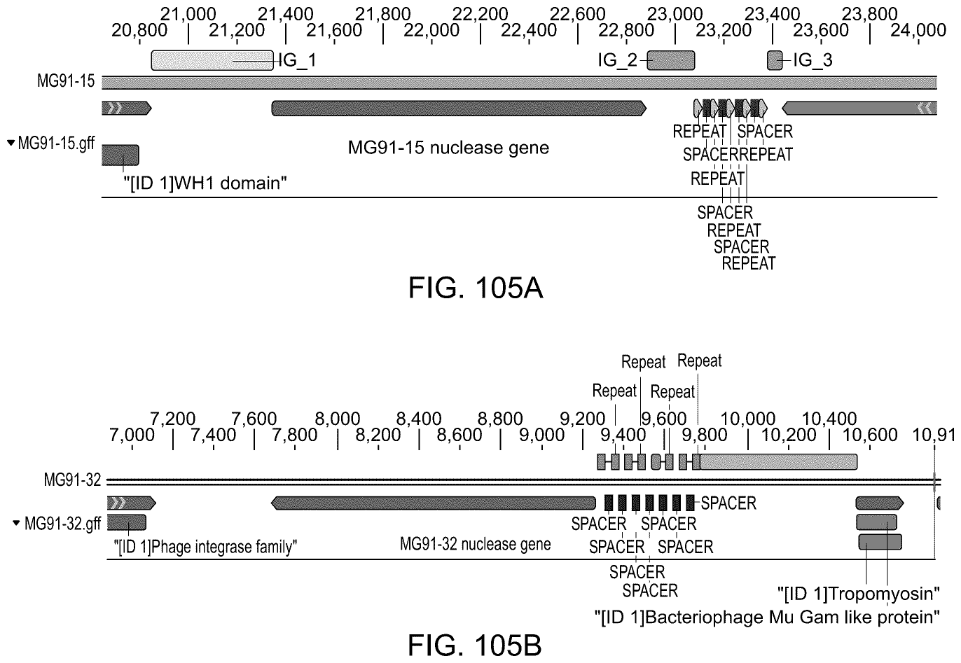

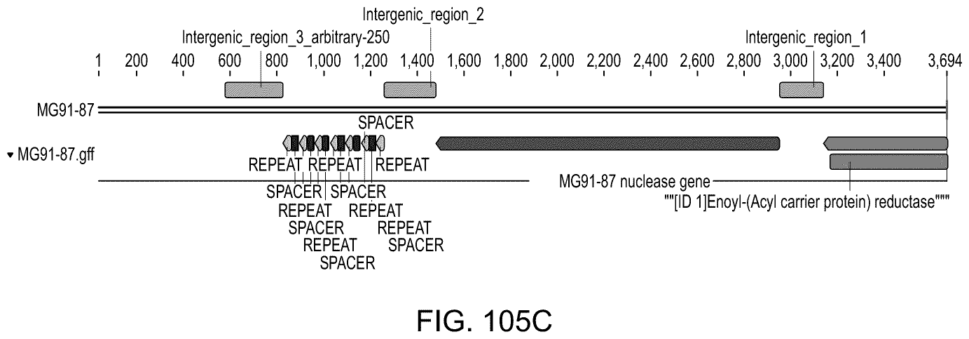

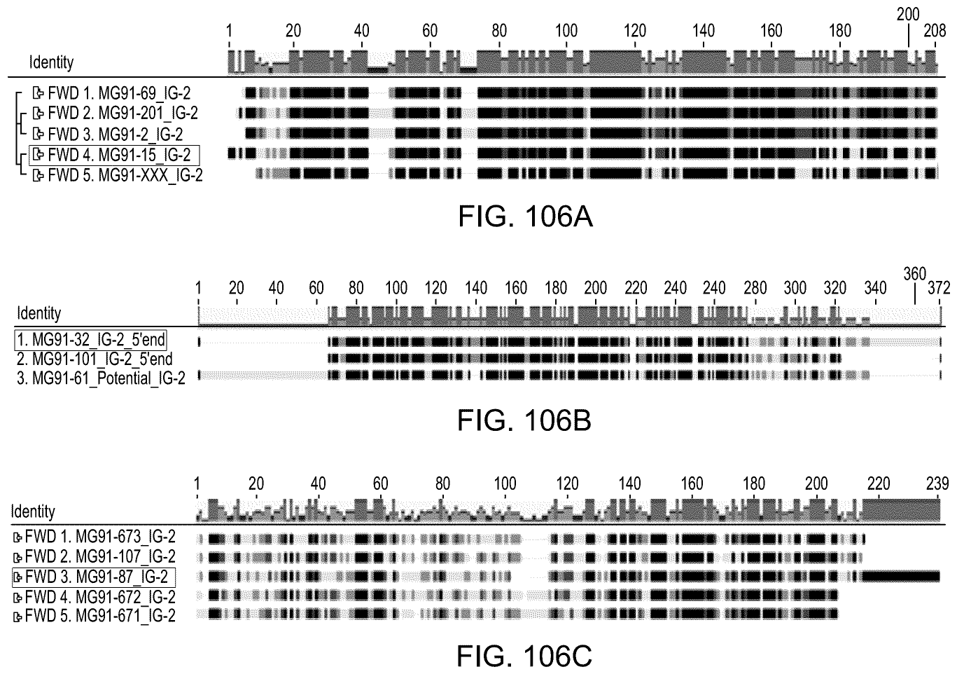

Compact type V nuclease proteins from distinct clades were targeted for in silico characterization of genomic regions encoding CRISPR Cas systems. To identify intergenic regions potentially encoding tracrRNAs, individual protein clades (with confirmed catalytic residues) were chosen for visual inspection of contigs encoding the compact type nuclease genes and a CRISPR array. Genomic regions devoid of coding sequence predictions between two genes, or between genes and CRISPR arrays, were manually annotated as intergenic regions (FIG. 105). Intergenic regions upstream and downstream from a nuclease gene as well as a CRISPR array, i.e., at the same location relative to a nuclease and the corresponding CRISPR array, were consistently assigned labels across contigs encoding homologous nucleases. Nucleotide sequences of matching intergenic regions were aligned and inspected for conserved motifs across sequences (FIG. 106). Similarly, nucleotide sequences from non-matching intergenic regions within clades were aligned and inspected. By comparison, intergenic regions with the highest degree of conservation among them were identified as potentially encoding tracrRNAs.

Intergenic regions potentially encoding tracrRNAs corresponding to candidate nucleases (SEQ ID NOs: 6040-6049) were identified by the methods described herein, and are the subject of in vitro characterization. Results for active nucleases MG91-15 (SEQ ID NO: 2824), MG91-32 (SEQ ID NO: 2841), and MG91-87 (SEQ ID NO: 28%) are presented herein.

Intergenic Region Secondary Structure Predictions Inform tracrRNA Prediction

Intergenic regions potentially encoding tracrRNAs were folded with the corresponding repeat sequences using different energy models (Turner 2004 or Andronescu 2007) and parameters (for example, 20° C., 37° C., dangling ends). The stability of potential secondary RNA structures was visually inspected based on base pairs probabilities. Optimal intergenic region/repeat folds for MG91-15, MG91-32, and MG91-87 were obtained and used to inform the design of single guide RNAs.

### MG91-15, MG91-32 and MG91-87sgRNA Design

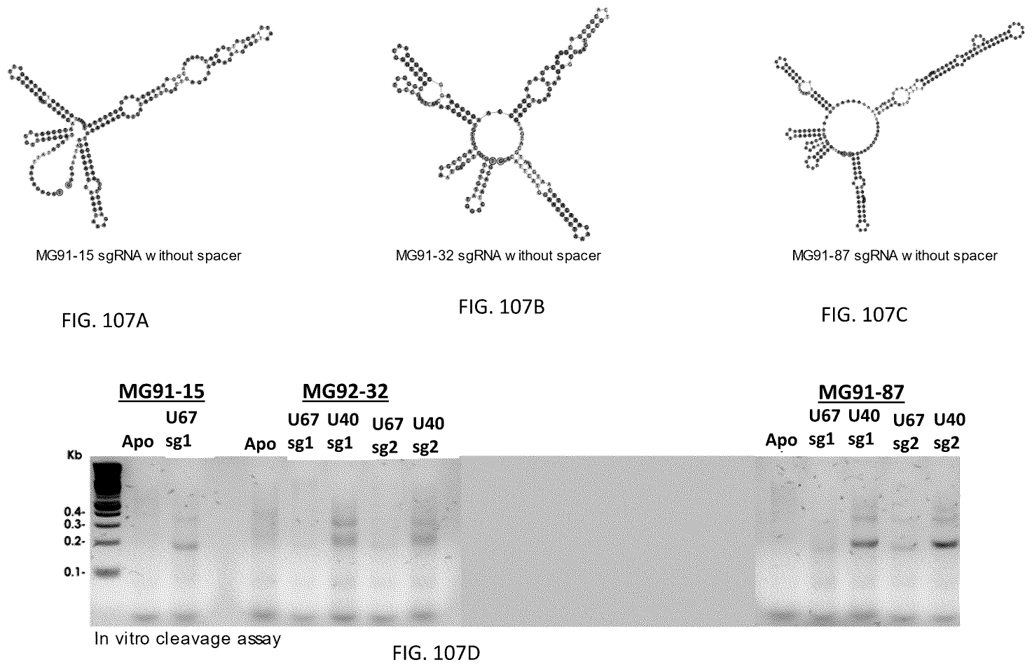

Promising folds between intergenic regions potentially containing tracrRNAs with the corresponding repeat sequences were modified as follows: tracrRNA sequences were trimmed on the 3′ end and sometimes on the 5′ end, and repeat sequences were trimmed on the 5′ end. Both RNA sequences were connected via a GAAA tetraloop and folded for secondary structure prediction as described above. The secondary structure for active sgRNAs corresponding to nucleases MG91-15 sgRNA1 (SEQ ID NO: 6033), MG91-32 sgRNA1 (SEQ ID NO: 6034), and MG91-87 sgRNA1 (SEQ ID NO: 6035) are shown in FIG. 107.

### In Vitro Activity and PAM Sequence Determination for MG91-15, MG91-32 and MG91-87 Nucleases

5 nM of nuclease amplified DNA templates and 25 nM sgRNA amplified DNA templates were expressed at 37° C. for 3 hours with PURExpress® In Vitro Protein Synthesis Kit (New England Biolabs Inc.). Plasmid library DNA cleavage reactions were carried out by mixing 5 nM of the target library representing all possible 8N PAMs, a 5-fold dilution of PURExpress expressions, 10 nM Tris-HCl, 10 nM MgCl2, and 100 mM NaCl at 37° C. for 2 hours. Reactions were stopped and cleaned with HighPrep™ PCR clean up beads (MAGBIO Genomics, Inc.) and eluted in Tris EDTA pH 8.0 buffer. 3 nM of the cleavage product ends were blunted with 3.33 μM dNTPs, 1× T4 DNA ligase buffer, and 0.167 U/μL of Klenow Fragment (New England Biolabs Inc.) at 25° C. for 15 minutes. 1.5 nM of the cleavage products were ligated with 150 nM adapters, 1× T4 DNA ligase buffer (New England Biolabs Inc.), and 20 U/μL T4 DNA ligase (New England Biolabs Inc.) at room temperature for 20 minutes. The ligated products were amplified by PCR with NGS primers and sequenced by NGS to obtain the PAM.

Active proteins that successfully cleaved the PAM library yielded a band around 188 or 205 bp in an agarose gel (FIG. 107D).

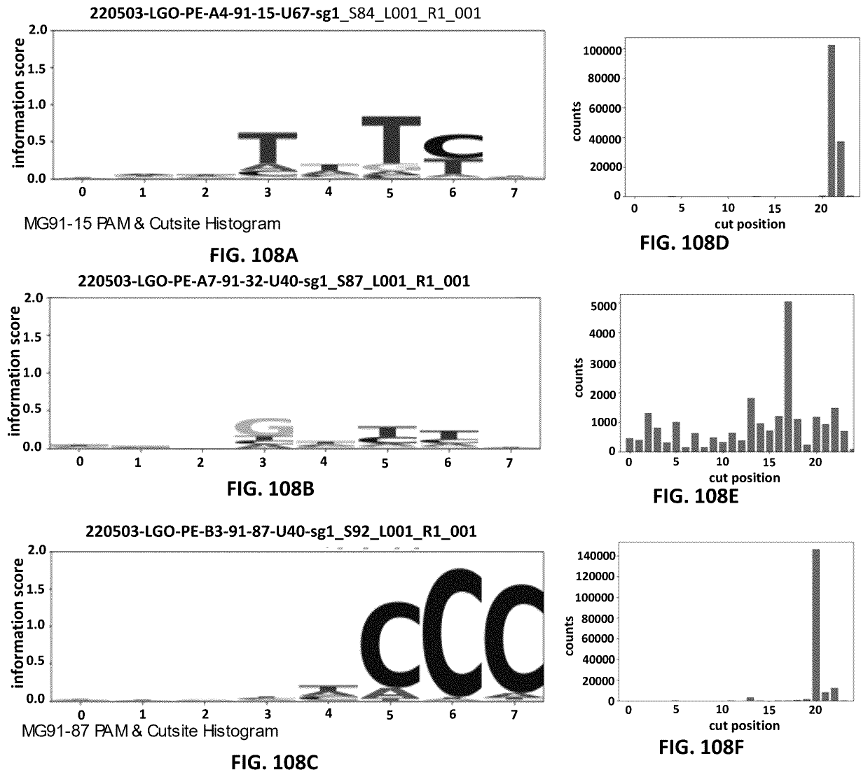

Sequence logos were made using Seqlogo maker, and histograms were made from the counts of reads at each nucleotide position. The PAM recognized by MG91-15 is a 5′-TtTYn sequence (FIG. 108A, Sequence Number: A6037), by MG91-32 is a 5′-GnYYn sequence (FIG. 108B,Sequence Number: A6038), and by MG-87 is a 5′-wCCC sequence (FIG. 108C, Sequence Number: A6039). The preferred cut position(s) were nucleotides 21 and 22 for MG91-15 (FIG. 108D), nucleotide 17 for MG91-32 (FIG. 108E) and nucleotide 20 for MG91-87 (FIG. 108F).

Covariance models were built as described above (In silico search for novel tracrRNA sequences, Compact type MG55-43 Cas nuclease system) from active tracrRNA sequences, and used to refine the prediction of tracrRNAs from other intergenic regions associated with nucleases in the MG91 family.

### In Vitro Activity of Compact Type V MG91 Nucleases and Selected Intergenic Regions (Prophetic)

Nuclease, intergenic sequences, and minimal arrays are expressed in transcription-translation reaction mixtures using myTXTL® Sigma 70 Master Mix Kit (Arbor Biosciences) as described before. Ribonucleoprotein complexes are tested in in vitro cleavage reactions of plasmid target DNA library using the TXTL expressions. The products are amplified by PCR with NGS primers and sequenced by NGS to obtain the PAM sequence.

Active proteins that successfully cleave the PAM library are expected to yield a band around 188 or 205 bp in agarose gel electrophoresis. Sequence logos are made using Seqlogo maker, and histograms are generated from the counts of reads at each nucleotide position.

To obtain the sequence of active tracrRNA and crRNA, RNA is extracted from TXTL lysate following the Quick-RNA™ Miniprep Kit (Zymo Research). The total concentration of the transcripts is measured on a Nanodrop, Tapestation, and Qubit; and is subjected to next generation sequencing.

The sequence of active tracrRNAs and crRNAs is used to design sgRNAs, and is subsequently tested in PURExpress with the corresponding MG91 nuclease to confirm the PAM sequence and cut site.

E. coli Expressions (Compact Type V Nucleases) (Prophetic)

Plasmids encoding the effector, intergenic sequence from the genomic contig, native repeat, and universal spacer sequences with a T7 promoter are transformed into BL21 DE3 or T7 Express 1ysY/Iq and cultured at 37° C. in 60 mL terrific broth media supplemented with 100 μg/mL of ampicillin. Expression is induced with 0.4 mM IPTG after cultures reach OD600 nm of 0.5 and cells are incubated at 16° C. overnight. 25 mL of cells are pelleted by centrifugation and resuspended in 1.5 mL of lysis buffer (20 mM Tris-HCl, 500 mM NaCl, 1 mM TCEP, 5% glycerol, 10 mM MgCl₂ pH 7.5 with Pierce Protease Inhibitor, (Thermo Scientific™)). Cells are then lysed by sonication. Supernatant and cell debris are separated by centrifugation.

In Vitro Cleavage Efficiency with Purified Protein (Compact Type V Nucleases) (Prophetic)

Proteins are expressed in E. coli protease deficient B strain under T7 inducible promoter, the cells are lysed using sonication, and the His-tagged protein of interest is purified using HisTrap FF (GE Lifescience) Ni-NTA affinity chromatography on the AKTA Avant FPLC (GE Lifescience). Purity is determined using densitometry in ImageLab software (Bio-Rad) of the protein bands resolved on SDS-PAGE and InstantBlue Ultrafast (Sigma-Aldrich) coomassie stained acrylamide gels (Bio-Rad). The protein is desalted in storage buffer composed of 50 mM Tris-HCl, 300 mM NaCl, 1 mM TCEP, 5% glycerol; pH 7.5 and stored at −80° C.

A target DNA is constructed that contains a spacer sequence and the PAM determined via NGS. In the case of degenerate bases in the PAM, a single representative PAM is chosen for testing. The target DNA is 2200 bp of linear DNA derived from a plasmid via PCR amplification. The PAM and spacer are located 700 bp from one end. Successful cleavage results in fragments of 700 and 1500 bp.

The target DNA, in vitro transcribed single RNA, and purified recombinant protein are combined in cleavage buffer (10 mM Tris, 100 mM NaCl, 10 mM MgCl₂) with an excess of protein and RNA and incubated for 5′ to 3 hours, usually 1 hr. The reaction is stopped via addition of RNAse A and incubation at 60° C. The reaction is resolved on a 1.2% TAE agarose gel and the fraction of cleaved target DNA is quantified in ImageLab software.

Activity in E. coli (Compact Type V Nucleases) (Prophetic)

For testing of nuclease activity in bacterial cells, strains are constructed with genome sequences containing a spacer target with corresponding PAM sequence specific to the enzyme of interest. Engineered strains are then transformed with the nuclease of interest and transformants are then subsequently made chemocompetent and transformed with 50 ng of single guides either specific to the target sequence (on target) or non specific to the target (off target). After heat shock, transformations are recovered in SOC for 2 hrs at 37° C., and nuclease efficiency is determined by a 5-fold dilution series grown on induction media. Colonies are quantified from the dilution series in triplicate. Nuclease, and therefore genome editing capability, is assessed by quantifying the reduction of viable cells in the presence of guides and nucleases targeting the host cell chromosome.

### Activity in Mammalian Cells (Compact Type V Nucleases) (Prophetic)

To show targeting and cleavage activity in mammalian cells, and therefore genome editing potential, the protein sequences are cloned into two mammalian expression vectors, one with a C-terminal SV40 NLS and a 2A-GFP tag and one with no GFP tag and two NLS sequences (one on the N-terminus and one on the C-terminus). Alternative NLS sequences that could also be used are listed in Table 45.

TABLE 45
Alternative nuclear localization sequences (NLS)

| Source | NLS amino acid sequence |
|---|---|
| SV40 | PKKKRKV |
| nucleoplasmin | KRPAATKKAGQAKKKK |
| bipartite NLS | |
| c-myc NLS | PAAKRVKLD |
| c-myc NLS | RQRRNELKRSP |
| hRNPA1 M9 NLS | NQSSNFGPMKGGNFGGRSSGPY |
| | GGGGQYFAKPRNQGGY |
| Importin-alpha | RMRIZFKNKGKDTAELRRRRVE |
| IBB domain | VSVELRKAKKDEQILKRRNV |
| Myoma T protein | VSRKRPRP |
| Myoma T protein | PPKKARED |
| p53 | PQPKKKPL |
| mouse c-abl IV | SALIKKKKKMAP |
| influenza virus NS1 | DRLRR |
| influenza virus NS1 | PKQKKRK |
| Hepatitis virus | RKLKKKIKKL |
| delta antigen | |
| mouse Mx1 protein | REKKKFLKRR |
| human poly(ADP-ribose) | KRKGDEVDGVDEVAKKKSKK |
| polymerase | |
| steroid hormone | RKCLQAGMNLEARKTKK |
| receptors | |
| (human) | |
| glucocorticoid | |

The DNA sequence for the protein can be the native sequence, the E. coli codon optimized sequence, or the mammalian codon optimized sequence. The single guide RNA sequence with a gene target of interest is also cloned into a mammalian expression vector. The two plasmids are cotransfected into HEK293T cells. 72 hr after co-transfection of the expression plasmid and a sgRNA targeting plasmid into HEK293T cells, the DNA is extracted and used for the preparation of an NGS library. Percent NHEJ is measured via InDels in the sequencing of the target site to demonstrate the targeting efficiency of the enzyme in mammalian cells. At least 10 different target sites are chosen for testing protein activity.

TABLE 46
Listing of additional protein and nucleic
acid sequences referred to herein not
included in the sequence listing

| | Se- | | | | | |
|---|---|---|---|---|---|---|
| | quence | SEQ | Descrip- | | Organ- | |
| Cat. | Number | ID: | tion | Type | ism | Sequence |
| | — | 4426 | MG29-1 | | | TAATACGACTCACTAT |
| | | | WT | | | AAGGAAAAGCCAGCTC |
| | | | mRNA | | | CAGCAGGCGCTGCTCA |
| | | | | | | CTCCTCCCCATCCTCT |
| | | | | | | CCCTCTGTCCCTCTGT |
| | | | | | | CCCTCTGACCCTGCAC |
| | | | | | | TGTCCCAGCACCATGG |
| | | | | | | CCCCCAAGAAGAAGCG |
| | | | | | | GAAAGTTGGCGGCGG |
| | | | | | | AGGCAGCTTCAACAAC |
| | | | | | | TTCATCAAGAAATACA |
| | | | | | | GCCTGCAGAAGACCCT |
| | | | | | | GCGGTTCGAACTGAAG |
| | | | | | | CCCGTGGGCGAGACA |
| | | | | | | GCGGACTACATCGAAG |
| | | | | | | ACTTCAAGAGCGAATA |
| | | | | | | CCTGAAGGACACGGTG |
| | | | | | | CTGAAGGACGAACAGC |
| | | | | | | GGGCAAAAGACTACCA |
| | | | | | | GGAGATCAAAACACTG |
| | | | | | | ATCGACGACTACCACC |
| | | | | | | GGGAGTACATCGAAGA |
| | | | | | | ATGCCTGAGGGAACCC |
| | | | | | | GTGGACAAAAAGACCG |
| | | | | | | GCGAGATCCTGGACTT |
| | | | | | | CACACAGGACCTGGAA |
| | | | | | | GACGCATTCAGCTACT |
| | | | | | | ACCAGAAACTGAAAGA |
| | | | | | | AAACCCCACCGAGAAC |
| | | | | | | CGAGTGGGGTGGGAG |
| | | | | | | AAAGAGCAGGAGAGC |
| | | | | | | CTGAGAAAGAAGCTGG |
| | | | | | | TGACCAGCTTCGTGGG |
| | | | | | | GAACGACGGCCTGTTC |
| | | | | | | AAGAAAGAGTTCATCA |
| | | | | | | CCCGCGACCTGCCCGA |
| | | | | | | ATGGCTGCAGAAAAAG |
| | | | | | | GGGCTGTGGGGCGAA |
| | | | | | | TACAAGGACACCGTGG |
| | | | | | | AGAACTTCAAAAAATT |
| | | | | | | CACCACCTACTTCAGC |
| | | | | | | GGCTTCCACGAGAACA |
| | | | | | | GGAAGAATATGTACAC |
| | | | | | | AGCCGAAGCCCAGAG |
| | | | | | | CACAGCCATCGCCAAC |
| | | | | | | AGGCTGATGAACGACA |
| | | | | | | ACCTGCCCAAGTTCTT |
| | | | | | | CAACAACTACCTGGCA |
| | | | | | | TACCAGACCATCAAGG |
| | | | | | | AGAAACACCCCGACCT |
| | | | | | | GGTGTTCCGACTGGAC |
| | | | | | | GACGCCCTGCTGCAGG |
| | | | | | | CCGCCGGCGTGGAGC |
| | | | | | | ACCTGGACGAGGCATT |
| | | | | | | CCAGCCCAGATACTTC |
| | | | | | | AGCAGACTGTTCGCAC |
| | | | | | | AGAGCGGAATCACGG |
| | | | | | | CCTTCAACGAGCTGAT |
| | | | | | | CGGAGGAAGGACCAC |
| | | | | | | GGAAAACGGCGAAAA |
| | | | | | | GATCCAGGGCCTGAAC |
| | | | | | | GAGCAGATCAACCTGT |
| | | | | | | ACAGACAGCAGAACCC |
| | | | | | | CGAGAAGGCCAAGGG |
| | | | | | | CTTCCCAAGATTCATG |
| | | | | | | CCCCTGTTCAAGCAAA |
| | | | | | | TCCTGAGCGACAGGGA |
| | | | | | | GACCCACAGCTTCCTG |
| | | | | | | CCCGACGCATTCGAAA |
| | | | | | | ACGACAAAGAGCTGCT |
| | | | | | | GCAGGCCCTGAGGGA |
| | | | | | | CTACGTGGACGCCGCC |
| | | | | | | ACCAGCGAAGAAGGA |
| | | | | | | ATGATCAGCCAACTGA |
| | | | | | | ACAAGGCCATGAACCA |
| | | | | | | GTTCGTGACCGCCGAC |
| | | | | | | CTGAAAAGGGTGTACA |
| | | | | | | TCAAAAGCGCCGCCCT |
| | | | | | | GACCAGCCTGAGCCAG |
| | | | | | | GAACTGTTCCACTTCT |
| | | | | | | TCGGCGTGATCAGCGA |
| | | | | | | CGCCATCGCGTGGTAC |
| | | | | | | GCCGAGAAGAGACTG |
| | | | | | | AGCCCCAAGAAAGCCC |
| | | | | | | AGGAGAGCTTCCTGAA |
| | | | | | | ACAGGAAGTGTACGCC |
| | | | | | | ATCGAAGAACTGAACC |
| | | | | | | AGGCCGTGGTGGGCT |
| | | | | | | ACATCGACCAGCTGGA |
| | | | | | | AGACCAGAGCGAGCT |
| | | | | | | GCAGCAGCTGCTGGTG |
| | | | | | | GACCTGCCAGACCCCC |
| | | | | | | AGAAACCAGTGAGCAG |
| | | | | | | CTTCATCCTGACCCAC |
| | | | | | | TGGCAAAAAAGCCAGG |
| | | | | | | AGCCGCTGCAGGCCGT |
| | | | | | | GATCGCGAAGGTGGA |
| | | | | | | ACCCCTGTTCGAACTG |
| | | | | | | GAGGAGCTGAGCAAA |
| | | | | | | AACAAACGGGCCCCGA |
| | | | | | | AACACGACAAGGACCA |
| | | | | | | GGGAGGGGAAGGCTT |
| | | | | | | CCAGCAGGTGGACGC |
| | | | | | | AATCAAGAACATGCTG |
| | | | | | | GACGCATTCATGGAGG |
| | | | | | | TGAGCCACGCCATCAA |
| | | | | | | GCCCCTGTACCTGGTG |
| | | | | | | AAGGGCCGGAAAGCA |
| | | | | | | ATCGACATGCCGGACG |
| | | | | | | TGGACACAGGATTCTA |
| | | | | | | CGCCGACTTCGCGGAG |
| | | | | | | GCATACAGCGCCTACG |
| | | | | | | AGCAAGTGACGGTGA |
| | | | | | | GCCTGTACAACAAGAC |
| | | | | | | CCGAAACCACCTGAGC |
| | | | | | | AAGAAACCCTTCAGCA |
| | | | | | | AAGACAAAATCAAAAT |
| | | | | | | CAACTTCGACGCCCCA |
| | | | | | | ACACTGCTGAACGGCT |
| | | | | | | GGGACCTGAACAAGG |
| | | | | | | AAAGCGACAACAAAAG |
| | | | | | | CATCATCCTGAGAAAA |
| | | | | | | GACGGAAACTTCTACC |
| | | | | | | TGGCCATCATGCACCC |
| | | | | | | CAAACACACAAAGGTG |
| | | | | | | TTCGACTGCTACAGCG |
| | | | | | | CCAGCGAGGCGGCCG |
| | | | | | | GGAAATGCTACGAGAA |
| | | | | | | AATGAACTACAAACTG |
| | | | | | | CTGAGCGGCGCCAACA |
| | | | | | | AGATGCTGCCCAAAGT |
| | | | | | | GTTCTTCAGCAAGAAG |
| | | | | | | GGAATCGAAACCTTCA |
| | | | | | | GCCCACCCCAGGAAAT |
| | | | | | | CCTGGACCTGTACAAG |
| | | | | | | AACAACGAACACAAGA |
| | | | | | | AGGGAGCCACCTTCAA |
| | | | | | | GCTGGAGAGCTGCCAC |
| | | | | | | AAGCTGATCGACTTCT |
| | | | | | | TCAAGCGGAACATCCC |
| | | | | | | CAAGTACAAGGTGCAC |
| | | | | | | CCAACCGACAACTTCG |
| | | | | | | GATGGGACGTCTTCGG |
| | | | | | | ATTCCACTTCAGCCCA |
| | | | | | | ACCAGCAGCTACGGCG |
| | | | | | | ACCTGAGCGGCTTCTA |
| | | | | | | CCGAGAGGTGGAAGC |
| | | | | | | CCAGGGGTACAAACTG |
| | | | | | | TGGTTCAGCGACGTGA |
| | | | | | | GCGAGGCATACATCAA |
| | | | | | | CAAGTGCGTGGAAGA |
| | | | | | | GGGCAAACTGTTCCTG |
| | | | | | | TTCCAGATCTACAACA |
| | | | | | | AGGACTTCAGCCCCAA |
| | | | | | | CAGCACCGGGAAGCC |
| | | | | | | AAACCTGCACACACTG |
| | | | | | | TACTGGAAAGGACTGT |
| | | | | | | TCGAACCCGAGAACCT |
| | | | | | | GAAGGACGTGGTGCT |
| | | | | | | GAAACTGAACGGCGA |
| | | | | | | GGCCGAGATCTTCTAC |
| | | | | | | AGGAAACACAGCATCA |
| | | | | | | AGCACGAGGACAAGA |
| | | | | | | CGATCCACCGGGCCAA |
| | | | | | | GGACCCAATCGCCAAC |
| | | | | | | AAAAACGCAGACAACC |
| | | | | | | CCAAGAAGCAGAGCGT |
| | | | | | | GTTCGACTACGACATC |
| | | | | | | ATCAAGGACAAGCGCT |
| | | | | | | ACACCCAGGACAAATT |
| | | | | | | CTTCTTCCACGTGCCC |
| | | | | | | ATCAGCCTGAACTTCA |
| | | | | | | AGAGCCAGGGAGTGG |
| | | | | | | TGCGGTTCAACGACAA |
| | | | | | | GATCAACGGCCTGCTG |
| | | | | | | GCCGCACAGGACGAC |
| | | | | | | GTGCACGTGATCGGGA |
| | | | | | | TCGACCGAGGGGAAC |
| | | | | | | GCCACCTGCTGTACTA |
| | | | | | | CACCGTGGTGAACGGC |
| | | | | | | AAGGGCGAGGTGGTG |
| | | | | | | GAACAGGGCAGCCTG |
| | | | | | | AACCAGGTGGCCACAG |
| | | | | | | ACCAGGGGTACGTGGT |
| | | | | | | GGACTACCAACAGAAA |
| | | | | | | CTGCACGCCAAAGAGA |
| | | | | | | AGGAGAGAGACCAGG |
| | | | | | | CCAGGAAGAACTGGA |
| | | | | | | GCACCATCGAAAACAT |
| | | | | | | CAAGGAGCTGAAGGC |
| | | | | | | CGGGTACCTGAGCCAG |
| | | | | | | GTGGTGCACAAACTGG |
| | | | | | | CCCAGCTGATCGTGAA |
| | | | | | | ACACAACGCCATCGTG |
| | | | | | | TGCCTGGAGGACCTGA |
| | | | | | | ACTTCGGATTCAAGAG |
| | | | | | | GGGACGGTTCAAAGTG |
| | | | | | | GAGAAGCAGGTGTACC |
| | | | | | | AGAAGTTCGAGAAAGC |
| | | | | | | CCTGATCGACAAGCTG |
| | | | | | | AACTACCTGGTGTTCA |
| | | | | | | AGGAACGGGGGGCCA |
| | | | | | | CCCAGGCAGGCGGAT |
| | | | | | | ACCTGAACGCCTACCA |
| | | | | | | GCTGGCCGCACCATTC |
| | | | | | | GAGAGCTTCGAAAAAC |
| | | | | | | TGGGCAAGCAGACCG |
| | | | | | | GCATCCTGTACTACGT |
| | | | | | | GCGGAGCGACTACACC |
| | | | | | | AGCAAGATCGACCCCG |
| | | | | | | CCACAGGCTTCGTGGA |
| | | | | | | CTTCCTGAAGCCCAAA |
| | | | | | | TACGAAAGCATGGCAA |
| | | | | | | AGAGCAAAGTGTTCTT |
| | | | | | | CGAGAGCTTCGAAAGA |
| | | | | | | ATCCAGTGGAACCAGG |
| | | | | | | CCAAAGGCTACTTCGA |
| | | | | | | GTTCGAATTCGACTAC |
| | | | | | | AAGAAAATGTGCCCCA |
| | | | | | | GCAGGAAGTTCGGCG |
| | | | | | | ACTACCGCACCCGGTG |
| | | | | | | GGTGGTGTGCACATTC |
| | | | | | | GGCGACACACGGTACC |
| | | | | | | AGAACAGGCGCAACAA |
| | | | | | | AAGCAGCGGCCAATG |
| | | | | | | GGAGACCGAGACAATC |
| | | | | | | GACGTGACCGCCCAGC |
| | | | | | | TGAAGGCCCTGTTCGC |
| | | | | | | GGCCTACGGCATCACC |
| | | | | | | TACAACCAGGAGGACA |
| | | | | | | ACATCAAGGACGCCAT |
| | | | | | | CGCAGCCGTGAAGTAC |
| | | | | | | ACAAAATTCTACAAAC |
| | | | | | | AGCTGTACTGGCTGCT |
| | | | | | | GAGACTGACGCTGAGC |
| | | | | | | CTGCGGCACAGCGTGA |
| | | | | | | CCGGGACCGACGAGG |
| | | | | | | ACTTCATCCTGAGCCC |
| | | | | | | CGTGGCCGACGAGAA |
| | | | | | | CGGCGTGTTCTTCGAC |
| | | | | | | AGCAGGAAGGCCACG |
| | | | | | | GACAAACAGCCCAAGG |
| | | | | | | ACGCAGACGCGAACG |
| | | | | | | GCGCCTACCACATCGC |
| | | | | | | CCTGAAGGGACTGTGG |
| | | | | | | AACCTGCAGCAGATCA |
| | | | | | | GGCAGCACGACTGGA |
| | | | | | | ACGTGGAAAAACCAAA |
| | | | | | | AAAGCTGAACCTGGCC |
| | | | | | | ATGAAAAACGAAGAGT |
| | | | | | | GGTTCGGCTTCGCACA |
| | | | | | | GAAGAAGAAATTCAGG |
| | | | | | | GCCTCTGGCGGAAAAA |
| | | | | | | GACCTGCCGCCACAAA |
| | | | | | | GAAAGCCGGACAGGC |
| | | | | | | CAAGAAAAAGAAGTGA |
| | | | | | | CCACACCCCCATTCCC |
| | | | | | | CCACTCCAGATAGAAC |
| | | | | | | TTCAGTTATATCTCAC |
| | | | | | | GTGTCTGGAGTTGGAT |
| | | | | | | CCAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAA |
| | — | 4427 | MG29-1 S168R | | | TAATACGACTCACTAT |
| | | | mRNA | | | AAGGAAAAGCCAGCTC |
| | | | | | | CAGCAGGCGCTGCTCA |
| | | | | | | CTCCTCCCCATCCTCT |
| | | | | | | CCCTCTGTCCCTCTGT |
| | | | | | | CCCTCTGACCCTGCAC |
| | | | | | | TGTCCCAGCACCATGG |
| | | | | | | CCCCCAAGAAGAAGCG |
| | | | | | | GAAAGTTGGCGGCGG |
| | | | | | | AGGCAGCTTCAACAAC |
| | | | | | | TTCATCAAGAAATACA |
| | | | | | | GCCTGCAGAAGACCCT |
| | | | | | | GCGGTTCGAACTGAAG |
| | | | | | | CCCGTGGGCGAGACA |
| | | | | | | GCGGACTACATCGAAG |
| | | | | | | ACTTCAAGAGCGAATA |
| | | | | | | CCTGAAGGACACGGTG |
| | | | | | | CTGAAGGACGAACAGC |
| | | | | | | GGGCAAAAGACTACCA |
| | | | | | | GGAGATCAAAACACTG |
| | | | | | | ATCGACGACTACCACC |
| | | | | | | GGGAGTACATCGAAGA |
| | | | | | | ATGCCTGAGGGAACCC |
| | | | | | | GTGGACAAAAAGACCG |
| | | | | | | GCGAGATCCTGGACTT |
| | | | | | | CACACAGGACCTGGAA |
| | | | | | | GACGCATTCAGCTACT |
| | | | | | | ACCAGAAACTGAAAGA |
| | | | | | | AAACCCCACCGAGAAC |
| | | | | | | CGAGTGGGGTGGGAG |
| | | | | | | AAAGAGCAGGAGAGC |
| | | | | | | CTGAGAAAGAAGCTGG |
| | | | | | | TGACCAGCTTCGTGGG |
| | | | | | | GAACGACGGCCTGTTC |
| | | | | | | AAGAAAGAGTTCATCA |
| | | | | | | CCCGCGACCTGCCCGA |
| | | | | | | ATGGCTGCAGAAAAAG |
| | | | | | | GGGCTGTGGGGCGAA |
| | | | | | | TACAAGGACACCGTGG |
| | | | | | | AGAACTTCAAAAAATT |
| | | | | | | CACCACCTACTTCAGG |
| | | | | | | GGCTTCCACGAGAACA |
| | | | | | | GGAAGAATATGTACAC |
| | | | | | | AGCCGAAGCCCAGAG |
| | | | | | | CACAGCCATCGCCAAC |
| | | | | | | AGGCTGATGAACGACA |
| | | | | | | ACCTGCCCAAGTTCTT |
| | | | | | | CAACAACTACCTGGCA |
| | | | | | | TACCAGACCATCAAGG |
| | | | | | | AGAAACACCCCGACCT |
| | | | | | | GGTGTTCCGACTGGAC |
| | | | | | | GACGCCCTGCTGCAGG |
| | | | | | | CCGCCGGCGTGGAGC |
| | | | | | | ACCTGGACGAGGCATT |
| | | | | | | CCAGCCCAGATACTTC |
| | | | | | | AGCAGACTGTTCGCAC |
| | | | | | | AGAGCGGAATCACGG |
| | | | | | | CCTTCAACGAGCTGAT |
| | | | | | | CGGAGGAAGGACCAC |
| | | | | | | GGAAAACGGCGAAAA |
| | | | | | | GATCCAGGGCCTGAAC |
| | | | | | | GAGCAGATCAACCTGT |
| | | | | | | ACAGACAGCAGAACCC |
| | | | | | | CGAGAAGGCCAAGGG |
| | | | | | | CTTCCCAAGATTCATG |
| | | | | | | CCCCTGTTCAAGCAAA |
| | | | | | | TCCTGAGCGACAGGGA |
| | | | | | | GACCCACAGCTTCCTG |
| | | | | | | CCCGACGCATTCGAAA |
| | | | | | | ACGACAAAGAGCTGCT |
| | | | | | | GCAGGCCCTGAGGGA |
| | | | | | | CTACGTGGACGCCGCC |
| | | | | | | ACCAGCGAAGAAGGA |
| | | | | | | ATGATCAGCCAACTGA |
| | | | | | | ACAAGGCCATGAACCA |
| | | | | | | GTTCGTGACCGCCGAC |
| | | | | | | CTGAAAAGGGTGTACA |
| | | | | | | TCAAAAGCGCCGCCCT |
| | | | | | | GACCAGCCTGAGCCAG |
| | | | | | | GAACTGTTCCACTTCT |
| | | | | | | TCGGCGTGATCAGCGA |
| | | | | | | CGCCATCGCGTGGTAC |
| | | | | | | GCCGAGAAGAGACTG |
| | | | | | | AGCCCCAAGAAAGCCC |
| | | | | | | AGGAGAGCTTCCTGAA |
| | | | | | | ACAGGAAGTGTACGCC |
| | | | | | | ATCGAAGAACTGAACC |
| | | | | | | AGGCCGTGGTGGGCT |
| | | | | | | ACATCGACCAGCTGGA |
| | | | | | | AGACCAGAGCGAGCT |
| | | | | | | GCAGCAGCTGCTGGTG |
| | | | | | | GACCTGCCAGACCCCC |
| | | | | | | AGAAACCAGTGAGCAG |
| | | | | | | CTTCATCCTGACCCAC |
| | | | | | | TGGCAAAAAAGCCAGG |
| | | | | | | AGCCGCTGCAGGCCGT |
| | | | | | | GATCGCGAAGGTGGA |
| | | | | | | ACCCCTGTTCGAACTG |
| | | | | | | GAGGAGCTGAGCAAA |
| | | | | | | AACAAACGGGCCCCGA |
| | | | | | | AACACGACAAGGACCA |
| | | | | | | GGGAGGGGAAGGCTT |
| | | | | | | CCAGCAGGTGGACGC |
| | | | | | | AATCAAGAACATGCTG |
| | | | | | | GACGCATTCATGGAGG |
| | | | | | | TGAGCCACGCCATCAA |
| | | | | | | GCCCCTGTACCTGGTG |
| | | | | | | AAGGGCCGGAAAGCA |
| | | | | | | ATCGACATGCCGGACG |
| | | | | | | TGGACACAGGATTCTA |
| | | | | | | CGCCGACTTCGCGGAG |
| | | | | | | GCATACAGCGCCTACG |
| | | | | | | AGCAAGTGACGGTGA |
| | | | | | | GCCTGTACAACAAGAC |
| | | | | | | CCGAAACCACCTGAGC |
| | | | | | | AAGAAACCCTTCAGCA |
| | | | | | | AAGACAAAATCAAAAT |
| | | | | | | CAACTTCGACGCCCCA |
| | | | | | | ACACTGCTGAACGGCT |
| | | | | | | GGGACCTGAACAAGG |
| | | | | | | AAAGCGACAACAAAAG |
| | | | | | | CATCATCCTGAGAAAA |
| | | | | | | GACGGAAACTTCTACC |
| | | | | | | TGGCCATCATGCACCC |
| | | | | | | CAAACACACAAAGGTG |
| | | | | | | TTCGACTGCTACAGCG |
| | | | | | | CCAGCGAGGCGGCCG |
| | | | | | | GGAAATGCTACGAGAA |
| | | | | | | AATGAACTACAAACTG |
| | | | | | | CTGAGCGGCGCCAACA |
| | | | | | | AGATGCTGCCCAAAGT |
| | | | | | | GTTCTTCAGCAAGAAG |
| | | | | | | GGAATCGAAACCTTCA |
| | | | | | | GCCCACCCCAGGAAAT |
| | | | | | | CCTGGACCTGTACAAG |
| | | | | | | AACAACGAACACAAGA |
| | | | | | | AGGGAGCCACCTTCAA |
| | | | | | | GCTGGAGAGCTGCCAC |
| | | | | | | AAGCTGATCGACTTCT |
| | | | | | | TCAAGCGGAACATCCC |
| | | | | | | CAAGTACAAGGTGCAC |
| | | | | | | CCAACCGACAACTTCG |
| | | | | | | GATGGGACGTCTTCGG |
| | | | | | | ATTCCACTTCAGCCCA |
| | | | | | | ACCAGCAGCTACGGCG |
| | | | | | | ACCTGAGCGGCTTCTA |
| | | | | | | CCGAGAGGTGGAAGC |
| | | | | | | CCAGGGGTACAAACTG |
| | | | | | | TGGTTCAGCGACGTGA |
| | | | | | | GCGAGGCATACATCAA |
| | | | | | | CAAGTGCGTGGAAGA |
| | | | | | | GGGCAAACTGTTCCTG |
| | | | | | | TTCCAGATCTACAACA |
| | | | | | | AGGACTTCAGCCCCAA |
| | | | | | | CAGCACCGGGAAGCC |
| | | | | | | AAACCTGCACACACTG |
| | | | | | | TACTGGAAAGGACTGT |
| | | | | | | TCGAACCCGAGAACCT |
| | | | | | | GAAGGACGTGGTGCT |
| | | | | | | GAAACTGAACGGCGA |
| | | | | | | GGCCGAGATCTTCTAC |
| | | | | | | AGGAAACACAGCATCA |
| | | | | | | AGCACGAGGACAAGA |
| | | | | | | CGATCCACCGGGCCAA |
| | | | | | | GGACCCAATCGCCAAC |
| | | | | | | AAAAACGCAGACAACC |
| | | | | | | CCAAGAAGCAGAGCGT |
| | | | | | | GTTCGACTACGACATC |
| | | | | | | ATCAAGGACAAGCGCT |
| | | | | | | ACACCCAGGACAAATT |
| | | | | | | CTTCTTCCACGTGCCC |
| | | | | | | ATCAGCCTGAACTTCA |
| | | | | | | AGAGCCAGGGAGTGG |
| | | | | | | TGCGGTTCAACGACAA |
| | | | | | | GATCAACGGCCTGCTG |
| | | | | | | GCCGCACAGGACGAC |
| | | | | | | GTGCACGTGATCGGGA |
| | | | | | | TCGACCGAGGGGAAC |
| | | | | | | GCCACCTGCTGTACTA |
| | | | | | | CACCGTGGTGAACGGC |
| | | | | | | AAGGGCGAGGTGGTG |
| | | | | | | GAACAGGGCAGCCTG |
| | | | | | | AACCAGGTGGCCACAG |
| | | | | | | ACCAGGGGTACGTGGT |
| | | | | | | GGACTACCAACAGAAA |
| | | | | | | CTGCACGCCAAAGAGA |
| | | | | | | AGGAGAGAGACCAGG |
| | | | | | | CCAGGAAGAACTGGA |
| | | | | | | GCACCATCGAAAACAT |
| | | | | | | CAAGGAGCTGAAGGC |
| | | | | | | CGGGTACCTGAGCCAG |
| | | | | | | GTGGTGCACAAACTGG |
| | | | | | | CCCAGCTGATCGTGAA |
| | | | | | | ACACAACGCCATCGTG |
| | | | | | | TGCCTGGAGGACCTGA |
| | | | | | | ACTTCGGATTCAAGAG |
| | | | | | | GGGACGGTTCAAAGTG |
| | | | | | | GAGAAGCAGGTGTACC |
| | | | | | | AGAAGTTCGAGAAAGC |
| | | | | | | CCTGATCGACAAGCTG |
| | | | | | | AACTACCTGGTGTTCA |
| | | | | | | AGGAACGGGGGGCCA |
| | | | | | | CCCAGGCAGGCGGAT |
| | | | | | | ACCTGAACGCCTACCA |
| | | | | | | GCTGGCCGCACCATTC |
| | | | | | | GAGAGCTTCGAAAAAC |
| | | | | | | TGGGCAAGCAGACCG |
| | | | | | | GCATCCTGTACTACGT |
| | | | | | | GCGGAGCGACTACACC |
| | | | | | | AGCAAGATCGACCCCG |
| | | | | | | CCACAGGCTTCGTGGA |
| | | | | | | CTTCCTGAAGCCCAAA |
| | | | | | | TACGAAAGCATGGCAA |
| | | | | | | AGAGCAAAGTGTTCTT |
| | | | | | | CGAGAGCTTCGAAAGA |
| | | | | | | ATCCAGTGGAACCAGG |
| | | | | | | CCAAAGGCTACTTCGA |
| | | | | | | GTTCGAATTCGACTAC |
| | | | | | | AAGAAAATGTGCCCCA |
| | | | | | | GCAGGAAGTTCGGCG |
| | | | | | | ACTACCGCACCCGGTG |
| | | | | | | GGTGGTGTGCACATTC |
| | | | | | | GGCGACACACGGTACC |
| | | | | | | AGAACAGGCGCAACAA |
| | | | | | | AAGCAGCGGCCAATG |
| | | | | | | GGAGACCGAGACAATC |
| | | | | | | GACGTGACCGCCCAGC |
| | | | | | | TGAAGGCCCTGTTCGC |
| | | | | | | GGCCTACGGCATCACC |
| | | | | | | TACAACCAGGAGGACA |
| | | | | | | ACATCAAGGACGCCAT |
| | | | | | | CGCAGCCGTGAAGTAC |
| | | | | | | ACAAAATTCTACAAAC |
| | | | | | | AGCTGTACTGGCTGCT |
| | | | | | | GAGACTGACGCTGAGC |
| | | | | | | CTGCGGCACAGCGTGA |
| | | | | | | CCGGGACCGACGAGG |
| | | | | | | ACTTCATCCTGAGCCC |
| | | | | | | CGTGGCCGACGAGAA |
| | | | | | | CGGCGTGTTCTTCGAC |
| | | | | | | AGCAGGAAGGCCACG |
| | | | | | | GACAAACAGCCCAAGG |
| | | | | | | ACGCAGACGCGAACG |
| | | | | | | GCGCCTACCACATCGC |
| | | | | | | CCTGAAGGGACTGTGG |
| | | | | | | AACCTGCAGCAGATCA |
| | | | | | | GGCAGCACGACTGGA |
| | | | | | | ACGTGGAAAAACCAAA |
| | | | | | | AAAGCTGAACCTGGCC |
| | | | | | | ATGAAAAACGAAGAGT |
| | | | | | | GGTTCGGCTTCGCACA |
| | | | | | | GAAGAAGAAATTCAGG |
| | | | | | | GCCTCTGGCGGAAAAA |
| | | | | | | GACCTGCCGCCACAAA |
| | | | | | | GAAAGCCGGACAGGC |
| | | | | | | CAAGAAAAAGAAGTGA |
| | | | | | | CCACACCCCCATTCCC |
| | | | | | | CCACTCCAGATAGAAC |
| | | | | | | TTCAGTTATATCTCAC |
| | | | | | | GTGTCTGGAGTTGGAT |
| | | | | | | CCAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAA |
| MG29-1 | — | 4970 | MG29-1-hRosa26- | Nucleotide | N.A. | /AltR1/rUrArArUrUrUrCr |
| sgRNA | | | sgRNA-A1 | | | UrArCrUrGrUrUrGrUrAr |
| targeting | | | | | | GrArUrArGrGrUrCrArC |
| hRosa26 | | | | | | rUrGrUrCrCrUrArGrCrU |
| | | | | | | rCrUrCrCrA/AltR2/ |
| MG29-1 | — | 4971 | MG29-1-hRosa26- | Nucleotide | N.A. | /AltR1/rUrArArUrUrUrCr |
| sgRNA | | | sgRNA-B1 | | | UrArCrUrGrUrUrGrUrAr |
| targeting | | | | | | GrArUrUrUrUrUrUrArGr |
| hRosa26 | | | | | | GrCrCrGrGrGrCrGrCrG |
| | | | | | | rGrUrGrGrC/AltR2/ |
| MG29-1 | — | 4972 | MG29-1-hRosa26- | Nucleotide | N.A. | /AltR1/rUrArArUrUrUrCr |
| sgRNA | | | sgRNA-C1 | | | UrArCrUrGrUrUrGrUrAr |
| targeting | | | | | | GrArUrUrArGrGrCrCrG |
| hRosa26 | | | | | | rGrGrCrGrCrGrGrUrGr |
| | | | | | | GrCrUrCrArC/AltR2/ |
| MG29-1 | — | 4973 | MG29-1-hRosa26- | Nucleotide | N.A. | /AltR1/rUrArArUrUrUrCr |
| sgRNA | | | sgRNA-D1 | | | UrArCrUrGrUrUrGrUrAr |
| targeting | | | | | | GrArUrArGrGrCrCrGrG |
| hRosa26 | | | | | | rGrCrGrCrGrGrUrGrGr |
| | | | | | | CrUrCrArCrA/AltR2/ |
| MG29-1 | — | 4974 | MG29-1-hRosa26- | Nucleotide | N.A. | /AltR1/rUrArArUrUrUrCr |
| sgRNA | | | sgRNA-E1 | | | UrArCrUrGrUrUrGrUrAr |
| targeting | | | | | | GrArUrGrGrCrCrGrGrG |
| hRosa26 | | | | | | rCrGrCrGrGrUrGrGrCr |
| | | | | | | UrCrArCrArC/AltR2/ |
| MG29-1 | — | 4975 | MG29-1-hRosa26- | Nucleotide | N.A | /AltR1/rUrArArUrUrUrCr |
| sgRNA | | | sgRNA-F1 | | | UrArCrUrGrUrUrGrUrAr |
| targeting | | | | | | GrArUrGrGrArGrGrCrC |
| hRosa26 | | | | | | rGrArGrGrCrArGrGrCr |
| | | | | | | ArGrArUrCrA/AltR2/ |
| MG29-1 | — | 4976 | MG29-1-hRosa26- | Nucleotide | N.A. | /AltR1/rUrArArUrUrUrCr |
| sgRNA | | | sgRNA-G1 | | | UrArCrUrGrUrUrGrUrAr |
| targeting | | | | | | GrArUrArGrGrUrCrArG |
| hRosa26 | | | | | | rGrArGrUrUrCrArArGr |
| | | | | | | ArCrCrArGrC/AltR2/ |
| MG29-1 | — | 4977 | MG29-1-hRosa26- | Nucleotide | N.A. | /AltR1/rUrArArUrUrUrCr |
| sgRNA | | | sgRNA-H1 | | | UrArCrUrGrUrUrGrUrAr |
| targeting | | | | | | GrArUrCrArGrUrGrArG |
| hRosa26 | | | | | | rCrUrGrArGrArUrCrGr |
| | | | | | | UrGrCrCrArU/AltR2/ |
| MG29-1 | — | 4978 | MG29-1-hRosa26- | Nucleotide | N.A. | /AltR1/rUrArArUrUrUrCr |
| sgRNA | | | sgRNA-A2 | | | UrArCrUrGrUrUrGrUrAr |
| targeting | | | | | | GrArUrUrUrUrUrUrUrGr |
| hRosa26 | | | | | | GrUrUrGrGrGrUrGrUrG |
| | | | | | | rGrUrGrGrC/AltR2/ |
| MG29-1 | — | 4979 | MG29-1-hRosa26- | Nucleotide | N.A. | /AltR1/rUrArArUrUrUrCr |
| sgRNA | | | sgRNA-B2 | | | UrArCrUrGrUrUrGrUrAr |
| targeting | | | | | | GrArUrUrUrGrGrUrUrG |
| hRosa26 | | | | | | rGrGrUrGrUrGrGrUrGr |
| | | | | | | GrCrUrCrArC/AltR2/ |
| MG29-1 | — | 4980 | MG29-1-hRosa26- | Nucleotide | N.A. | /AltR1/rUrArArUrUrUrCr |
| sgRNA | | | sgRNA-C2 | | | UrArCrUrGrUrUrGrUrAr |
| targeting | | | | | | GrArUrUrGrGrUrUrGrG |
| hRosa26 | | | | | | rGrUrGrUrGrGrUrGrGr |
| | | | | | | CrUrCrArCrA/AltR2/ |
| MG29-1 | — | 4981 | MG29-1-hRosa26- | Nucleotide | N.A. | /AltR1/rUrArArUrUrUrCr |
| sgRNA | | | sgRNA-D2 | | | UrArCrUrGrUrUrGrUrAr |
| targeting | | | | | | GrArUrGrGrUrUrGrGrG |
| hRosa26 | | | | | | rUrGrUrGrGrUrGrGrCr |
| | | | | | | UrCrArCrArC/AltR2/ |
| MG29-1 | — | 4982 | MG29-1-hRosa26- | Nucleotide | N.A. | /AltR1/rUrArArUrUrUrCr |
| sgRNA | | | sgRNA-E2 | | | UrArCrUrGrUrUrGrUrAr |
| targeting | | | | | | GrArUrGrUrUrGrGrGrU |
| hRosa26 | | | | | | rGrUrGrGrUrGrGrCrUr |
| | | | | | | CrArCrArCrC/AltR2/ |
| MG29-1 | — | 4983 | MG29-1-hRosa26- | Nucleotide | N.A. | /AltR1/rUrArArUrUrUrCr |
| sgRNA | | | sgRNA-F2 | | | UrArCrUrGrUrUrGrUrAr |
| targeting | | | | | | GrArUrGrArArGrGrArU |
| hRosa26 | | | | | | rGrArGrGrCrArGrArAr |
| | | | | | | GrGrArUrCrA/AltR2/ |
| MG29-1 | — | 4984 | MG29-1-hRosa26- | Nucleotide | N.A. | /AltR1/rUrArArUrUrUrCr |
| sgRNA | | | sgRNA-G2 | | | UrArCrUrGrUrUrGrUrAr |
| targeting | | | | | | GrArUrGrGrArGrGrCrC |
| hRosa26 | | | | | | rArArGrGrCrGrGrGrCr |
| | | | | | | GrGrArUrCrA/AltR2/ |
| MG29-1 | — | 4985 | MG29-1-hRosa26- | Nucleotide | N.A. | /AltR1/rUrArArUrUrUrCr |
| sgRNA | | | sgRNA-H2 | | | UrArCrUrGrUrUrGrUrAr |
| targeting | | | | | | GrArUrCrGrCrCrArUrUr |
| hRosa26 | | | | | | CrUrCrCrUrGrCrCrUrCr |
| | | | | | | ArGrCrCrU/AltR2/ |
| MG29-1 | — | 4986 | MG29-1-hRosa26- | Nucleotide | N.A. | /AltR1/rUrArArUrUrUrCr |
| sgRNA | | | sgRNA-A3 | | | UrArCrUrGrUrUrGrUrAr |
| targeting | | | | | | GrArUrUrUrGrUrGrUrU |
| hRosa26 | | | | | | rUrUrUrArGrUrArGrAr |
| | | | | | | GrArArGrGrG/AltR2/ |
| MG29-1 | — | 4987 | MG29-1-hRosa26- | Nucleotide | N.A. | /AltR1/rUrArArUrUrUrCr |
| sgRNA | | | sgRNA-B3 | | | UrArCrUrGrUrUrGrUrAr |
| targeting | | | | | | GrArUrUrGrUrGrUrUrU |
| hRosa26 | | | | | | rUrUrArGrUrArGrArGr |
| | | | | | | ArArGrGrGrG/AltR2/ |
| MG29-1 | — | 4988 | MG29-1-hRosa26- | Nucleotide | N.A. | /AltR1/rUrArArUrUrUrCr |
| sgRNA | | | sgRNA-C3 | | | UrArCrUrGrUrUrGrUrAr |
| targeting | | | | | | GrArUrGrUrGrUrUrUrU |
| hRosa26 | | | | | | rUrArGrUrArGrArGrAr |
| | | | | | | ArGrGrGrGrU/AltR2/ |
| MG29-1 | — | 4989 | MG29-1-hRosa26- | Nucleotide | N.A. | /AltR1/rUrArArUrUrUrCr |
| sgRNA | | | sgRNA-D3 | | | UrArCrUrGrUrUrGrUrAr |
| targeting | | | | | | GrArUrUrGrUrUrUrUrUr |
| hRosa26 | | | | | | ArGrUrArGrArGrArArG |
| | | | | | | rGrGrGrUrU/AltR2/ |
| MG29-1 | — | 4990 | MG29-1-hRosa26- | Nucleotide | N.A. | /AltR1/rUrArArUrUrUrCr |
| sgRNA | | | sgRNA-E3 | | | UrArCrUrGrUrUrGrUrAr |
| targeting | | | | | | GrArUrUrArGrUrArGrA |
| hRosa26 | | | | | | rGrArArGrGrGrGrUrUr |
| | | | | | | UrCrArCrCrG/AltR2/ |
| MG29-1 | — | 4991 | MG29-1-hRosa26- | Nucleotide | N.A. | /AltR1/rUrArArUrUrUrCr |
| sgRNA | | | sgRNA-F3 | | | UrArCrUrGrUrUrGrUrAr |
| targeting | | | | | | GrArUrArGrUrArGrArG |
| hRosa26 | | | | | | rArArGrGrGrGrUrUrUr |
| | | | | | | CrArCrCrGrU/AltR2/ |
| MG29-1 | — | 4992 | MG29-1-hRosa26- | Nucleotide | N.A. | /AltR1/rUrArArUrUrUrCr |
| sgRNA | | | sgRNA-G3 | | | UrArCrUrGrUrUrGrUrAr |
| targeting | | | | | | GrArUrGrUrArGrArGrA |
| hRosa26 | | | | | | rArGrGrGrGrUrUrUrCr |
| | | | | | | ArCrCrGrUrG/AltR2/ |
| MG29-1 | — | 4993 | MG29-1-hRosa26- | Nucleotide | N.A. | /AltR1/rUrArArUrUrUrCr |
| sgRNA | | | sgRNA-H3 | | | UrArCrUrGrUrUrGrUrAr |
| targeting | | | | | | GrArUrArCrCrGrUrGrU |
| hRosa26 | | | | | | rUrArGrCrCrArGrGrAr |
| | | | | | | UrGrGrUrCrU/AltR2/ |
| MG29-1 | — | 4994 | MG29-1-hRosa26- | Nucleotide | N.A. | /AltR1/rUrArArUrUrUrCr |
| sgRNA | | | sgRNA-A4 | | | UrArCrUrGrUrUrGrUrAr |
| targeting | | | | | | GrArUrGrArArCrUrCrCr |
| hRosa26 | | | | | | UrUrGrGrCrUrCrArArG |
| | | | | | | rUrGrArUrC/AltR2/ |
| MG29-1 | — | 4995 | MG29-1-hRosa26- | Nucleotide | N.A. | /AltR1/rUrArArUrUrUrCr |
| sgRNA | | | sgRNA-B4 | | | UrArCrUrGrUrUrGrUrAr |
| targeting | | | | | | GrArUrArArCrUrCrCrUr |
| hRosa26 | | | | | | UrGrGrCrUrCrArArGrU |
| | | | | | | rGrArUrCrC/AltR2/ |
| MG29-1 | — | 4996 | MG29-1-hRosa26- | Nucleotide | N.A. | /AltR1/rUrArArUrUrUrCr |
| sgRNA | | | sgRNA-C4 | | | UrArCrUrGrUrUrGrUrAr |
| targeting | | | | | | GrArUrUrUrUrUrUrUrUr |
| hRosa26 | | | | | | UrCrUrUrGrArGrUrCrAr |
| | | | | | | GrArGrUrC/AltR2/ |
| MG29-1 | — | 4997 | MG29-1-hRosa26- | Nucleotide | N.A. | /AltR1/rUrArArUrUrUrCr |
| sgRNA | | | sgRNA-D4 | | | UrArCrUrGrUrUrGrUrAr |
| targeting | | | | | | GrArUrUrUrUrUrCrUrUr |
| hRosa26 | | | | | | GrArGrUrCrArGrArGrU |
| | | | | | | rCrUrUrGrC/AltR2/ |
| MG29-1 | — | 4998 | MG29-1-hRosa26- | Nucleotide | N.A. | /AltR1/rUrArArUrUrUrCr |
| sgRNA | | | sgRNA-E4 | | | UrArCrUrGrUrUrGrUrAr |
| targeting | | | | | | GrArUrUrUrUrCrUrUrGr |
| hRosa26 | | | | | | ArGrUrCrArGrArGrUrC |
| | | | | | | rUrUrGrCrU/AltR2/ |
| MG29-1 | — | 4999 | MG29-1-hRosa26- | Nucleotide | N.A. | /AltR1/rUrArArUrUrUrCr |
| sgRNA | | | sgRNA-F4 | | | UrArCrUrGrUrUrGrUrAr |
| targeting | | | | | | GrArUrUrUrCrUrUrGrAr |
| hRosa26 | | | | | | GrUrCrArGrArGrUrCrU |
| | | | | | | rUrGrCrUrC/AltR2/ |
| MG29-1 | — | 5000 | MG29-1-hRosa26- | Nucleotide | N.A. | /AltR1/rUrArArUrUrUrCr |
| sgRNA | | | sgRNA-G4 | | | UrArCrUrGrUrUrGrUrAr |
| targeting | | | | | | GrArUrUrCrUrUrGrArG |
| hRosa26 | | | | | | rUrCrArGrArGrUrCrUrU |
| | | | | | | rGrCrUrCrC/AltR2/ |
| MG29-1 | — | 5001 | MG29-1-hRosa26- | Nucleotide | N.A. | /AltR1/rUrArArUrUrUrCr |
| sgRNA | | | sgRNA-H4 | | | UrArCrUrGrUrUrGrUrAr |
| targeting | | | | | | GrArUrCrUrUrGrArGrU |
| hRosa26 | | | | | | rCrArGrArGrUrCrUrUr |
| | | | | | | GrCrUrCrCrG/AltR2/ |
| MG29-1 | — | 5002 | MG29-1-hRosa26- | Nucleotide | N.A. | /AltR1/rUrArArUrUrUrCr |
| sgRNA | | | sgRNA-A5 | | | UrArCrUrGrUrUrGrUrAr |
| targeting | | | | | | GrArUrUrUrGrArGrUrC |
| hRosa26 | | | | | | rArGrArGrUrCrUrUrGr |
| | | | | | | CrUrCrCrGrU/AltR2/ |
| MG29-1 | — | 5003 | MG29-1-hRosa26- | Nucleotide | N.A. | /AltR1/rUrArArUrUrUrCr |
| sgRNA | | | sgRNA-B5 | | | UrArCrUrGrUrUrGrUrAr |
| targeting | | | | | | GrArUrUrGrUrArUrUrUr |
| hRosa26 | | | | | | UrUrArGrUrArGrArGrA |
| | | | | | | rUrGrGrGrG/AltR2/ |
| MG29-1 | — | 5004 | MG29-1-hRosa26- | Nucleotide | N.A. | /AltR1/rUrArArUrUrUrCr |
| sgRNA | | | sgRNA-C5 | | | UrArCrUrGrUrUrGrUrAr |
| targeting | | | | | | GrArUrGrUrArUrUrUrUr |
| hRosa26 | | | | | | UrArGrUrArGrArGrArU |
| | | | | | | rGrGrGrGrU/AltR2/ |
| MG29-1 | — | 5005 | MG29-1-hRosa26- | Nucleotide | N.A. | /AltR1/rUrArArUrUrUrCr |
| sgRNA | | | sgRNA-D5 | | | UrArCrUrGrUrUrGrUrAr |
| targeting | | | | | | GrArUrUrArUrUrUrUrUr |
| hRosa26 | | | | | | ArGrUrArGrArGrArUrG |
| | | | | | | rGrGrGrUrU/AltR2/ |
| MG29-1 | — | 5006 | MG29-1-hRosa26- | Nucleotide | N.A. | /AltR1/rUrArArUrUrUrCr |
| sgRNA | | | sgRNA-E5 | | | UrArCrUrGrUrUrGrUrAr |
| targeting | | | | | | GrArUrUrArGrUrArGrA |
| hRosa26 | | | | | | rGrArUrGrGrGrGrUrUr |
| | | | | | | UrCrArCrCrA/AltR2/ |
| MG29-1 | — | 5007 | MG29-1-hRosa26- | Nucleotide | N.A. | /AltR1/rUrArArUrUrUrCr |
| sgRNA | | | sgRNA-F5 | | | UrArCrUrGrUrUrGrUrAr |
| targeting | | | | | | GrArUrArGrUrArGrArG |
| hRosa26 | | | | | | rArUrGrGrGrGrUrUrUr |
| | | | | | | CrArCrCrArC/AltR2/ |
| MG29-1 | — | 5008 | MG29-1-hRosa26- | Nucleotide | N.A. | /AltR1/rUrArArUrUrUrCr |
| sgRNA | | | sgRNA-G5 | | | UrArCrUrGrUrUrGrUrAr |
| targeting | | | | | | GrArUrGrUrArGrArGrA |
| hRosa26 | | | | | | rUrGrGrGrGrUrUrUrCr |
| | | | | | | ArCrCrArCrG/AltR2/ |
| MG29-1 | — | 5009 | MG29-1-hRosa26- | Nucleotide | N.A. | /AltR1/rUrArArUrUrUrCr |
| sgRNA | | | sgRNA-H5 | | | UrArCrUrGrUrUrGrUrAr |
| targeting | | | | | | GrArUrArCrCrArCrGrUr |
| hRosa26 | | | | | | UrGrGrUrCrArGrGrCrU |
| | | | | | | rGrGrUrCrU/AltR2/ |
| MG29-1 | — | 5010 | MG29-1-hRosa26- | Nucleotide | N.A. | /AltR1/rUrArArUrUrUrCr |
| sgRNA | | | sgRNA-A6 | | | UrArCrUrGrUrUrGrUrAr |
| targeting | | | | | | GrArUrArCrCrUrUrCrCr |
| hRosa26 | | | | | | UrGrUrGrArCrUrUrCrCr |
| | | | | | | UrGrGrArG/AltR2/ |
| MG29-1 | — | 5011 | MG29-1-hRosa26- | Nucleotide | N.A. | /AltR1/rUrArArUrUrUrCr |
| sgRNA | | | sgRNA-B6 | | | UrArCrUrGrUrUrGrUrAr |
| targeting | | | | | | GrArUrCrCrUrUrCrCrUr |
| hRosa26 | | | | | | GrUrGrArCrUrUrCrCrUr |
| | | | | | | GrGrArGrA/AltR2/ |
| MG29-1 | — | 5012 | MG29-1-hRosa26- | Nucleotide | N.A. | /AltR1/rUrArArUrUrUrCr |
| sgRNA | | | sgRNA-C6 | | | UrArCrUrGrUrUrGrUrAr |
| targeting | | | | | | GrArUrUrGrGrGrGrArA |
| hRosa26 | | | | | | rGrGrUrUrCrUrCrUrGr |
| | | | | | | UrCrUrGrCrC/AltR2/ |
| DNA | — | 5013 | MG29-1-hRosa26- | Nucleotide | N.A. | AGGTCACTGTCCTAGC |
| sequence | | | target site-A1 | | | TCTCCA |
| of | | | | | | |
| hRosa26 | | | | | | |
| target | | | | | | |
| site | | | | | | |
| DNA | — | 5014 | MG29-1-hRosa26- | Nucleotide | N.A. | TTTTTAGGCCGGGCGC |
| sequence | | | target site-B1 | | | GGTGGC |
| of | | | | | | |
| hRosa26 | | | | | | |
| target | | | | | | |
| site | | | | | | |
| DNA | — | 5015 | MG29-1-hRosa26- | Nucleotide | N.A. | TAGGCCGGGCGCGGT |
| sequence | | | target site-C1 | | | GGCTCAC |
| of | | | | | | |
| hRosa26 | | | | | | |
| target | | | | | | |
| site | | | | | | |
| DNA | — | 5016 | MG29-1-hRosa26- | Nucleotide | N.A. | AGGCCGGGCGCGGTG |
| sequence | | | target site-D1 | | | GCTCACA |
| of | | | | | | |
| hRosa26 | | | | | | |
| target | | | | | | |
| site | | | | | | |
| DNA | — | 5017 | MG29-1-hRosa26- | Nucleotide | N.A. | GGCCGGGCGCGGTGG |
| sequence | | | target site-E1 | | | CTCACAC |
| of | | | | | | |
| hRosa26 | | | | | | |
| target | | | | | | |
| site | | | | | | |
| DNA | — | 5018 | MG29-1-hRosa26- | Nucleotide | N.A. | GGAGGCCGAGGCAGG |
| sequence | | | target site-F1 | | | CAGATCA |
| of | | | | | | |
| hRosa26 | | | | | | |
| target | | | | | | |
| site | | | | | | |
| DNA | — | 5019 | MG29-1-hRosa26- | Nucleotide | N.A. | AGGTCAGGAGTTCAAG |
| sequence | | | target site-G1 | | | ACCAGC |
| of | | | | | | |
| hRosa26 | | | | | | |
| target | | | | | | |
| site | | | | | | |
| DNA | — | 5020 | MG29-1-hRosa26- | Nucleotide | N.A. | CAGTGAGCTGAGATCG |
| sequence | | | target site-H1 | | | TGCCAT |
| of | | | | | | |
| hRosa26 | | | | | | |
| target | | | | | | |
| site | | | | | | |
| DNA | — | 5021 | MG29-1-hRosa26- | Nucleotide | N.A. | TTTTTTGGTTGGGTGT |
| sequence | | | target site-A2 | | | GGTGGC |
| of | | | | | | |
| hRosa26 | | | | | | |
| target | | | | | | |
| site | | | | | | |
| DNA | — | 5022 | MG29-1-hRosa26- | Nucleotide | N.A. | TTGGTTGGGTGTGGTG |
| sequence | | | target site-B2 | | | GCTCAC |
| of | | | | | | |
| hRosa26 | | | | | | |
| target | | | | | | |
| site | | | | | | |
| DNA | — | 5023 | MG29-1-hRosa26- | Nucleotide | N.A. | TGGTTGGGTGTGGTGG |
| sequence | | | target site-C2 | | | CTCACA |
| of | | | | | | |
| hRosa26 | | | | | | |
| target | | | | | | |
| site | | | | | | |
| DNA | — | 5024 | MG29-1-hRosa26- | Nucleotide | N.A. | GGTTGGGTGTGGTGG |
| sequence | | | target site-D2 | | | CTCACAC |
| of | | | | | | |
| hRosa26 | | | | | | |
| target | | | | | | |
| site | | | | | | |
| DNA | — | 5025 | MG29-1-hRosa26- | Nucleotide | N.A. | GTTGGGTGTGGTGGCT |
| sequence | | | target site-E2 | | | CACACC |
| of | | | | | | |
| hRosa26 | | | | | | |
| target | | | | | | |
| site | | | | | | |
| DNA | — | 5026 | MG29-1-hRosa26- | Nucleotide | N.A. | GAAGGATGAGGCAGA |
| sequence | | | target site-F2 | | | AGGATCA |
| of | | | | | | |
| hRosa26 | | | | | | |
| target | | | | | | |
| site | | | | | | |
| DNA | — | 5027 | MG29-1-hRosa26- | Nucleotide | N.A. | GGAGGCCAAGGCGGG |
| sequence | | | target site-G2 | | | CGGATCA |
| of | | | | | | |
| hRosa26 | | | | | | |
| target | | | | | | |
| site | | | | | | |
| DNA | — | 5028 | MG29-1-hRosa26- | Nucleotide | N.A. | CGCCATTCTCCTGCCT |
| sequence | | | target site-H2 | | | CAGCCT |
| of | | | | | | |
| hRosa26 | | | | | | |
| target | | | | | | |
| site | | | | | | |
| DNA | — | 5029 | MG29-1-hRosa26- | Nucleotide | N.A. | TTGTGTTTTTAGTAGA |
| sequence | | | target site-A3 | | | GAAGGG |
| of | | | | | | |
| hRosa26 | | | | | | |
| target | | | | | | |
| site | | | | | | |
| DNA | — | 5030 | MG29-1-hRosa26- | Nucleotide | N.A. | TGTGTTTTTAGTAGAG |
| sequence | | | target site-B3 | | | AAGGGG |
| of | | | | | | |
| hRosa26 | | | | | | |
| target | | | | | | |
| site | | | | | | |
| DNA | — | 5031 | MG29-1-hRosa26- | Nucleotide | N.A. | GTGTTTTTAGTAGAGA |
| sequence | | | target site-C3 | | | AGGGGT |
| of | | | | | | |
| hRosa26 | | | | | | |
| target | | | | | | |
| site | | | | | | |
| DNA | — | 5032 | MG29-1-hRosa26- | Nucleotide | N.A. | TGTTTTTAGTAGAGAA |
| sequence | | | target site-D3 | | | GGGGTT |
| of | | | | | | |
| hRosa26 | | | | | | |
| target | | | | | | |
| site | | | | | | |
| DNA | — | 5033 | MG29-1-hRosa26- | Nucleotide | N.A. | TAGTAGAGAAGGGGTT |
| sequence | | | target site-E3 | | | TCACCG |
| of | | | | | | |
| hRosa26 | | | | | | |
| target | | | | | | |
| site | | | | | | |
| DNA | — | 5034 | MG29-1-hRosa26- | Nucleotide | N.A. | AGTAGAGAAGGGGTTT |
| sequence | | | target site-F3 | | | CACCGT |
| of | | | | | | |
| hRosa26 | | | | | | |
| target | | | | | | |
| site | | | | | | |
| DNA | — | 5035 | MG29-1-hRosa26- | Nucleotide | N.A. | GTAGAGAAGGGGTTTC |
| sequence | | | target site-G3 | | | ACCGTG |
| of | | | | | | |
| hRosa26 | | | | | | |
| target | | | | | | |
| site | | | | | | |
| DNA | — | 5036 | MG29-1-hRosa26- | Nucleotide | N.A. | ACCGTGTTAGCCAGGA |
| sequence | | | target site-H3 | | | TGGTCT |
| of | | | | | | |
| hRosa26 | | | | | | |
| target | | | | | | |
| site | | | | | | |
| DNA | — | 5037 | MG29-1-hRosa26- | Nucleotide | N.A. | GAACTCCTTGGCTCAA |
| sequence | | | target site-A4 | | | GTGATC |
| of | | | | | | |
| hRosa26 | | | | | | |
| target | | | | | | |
| site | | | | | | |
| DNA | — | 5038 | MG29-1-hRosa26- | Nucleotide | N.A. | AACTCCTTGGCTCAAG |
| sequence | | | target site-B4 | | | TGATCC |
| of | | | | | | |
| hRosa26 | | | | | | |
| target | | | | | | |
| site | | | | | | |
| DNA | — | 5039 | MG29-1-hRosa26- | Nucleotide | N.A | TTTTTTTTCTTGAGTCA |
| sequence | | | target site-C4 | | | GAGTC |
| of | | | | | | |
| hRosa26 | | | | | | |
| target | | | | | | |
| site | | | | | | |
| DNA | — | 5040 | MG29-1-hRosa26- | Nucleotide | N.A. | TTTTCTTGAGTCAGAG |
| sequence | | | target site-D4 | | | TCTTGC |
| of | | | | | | |
| hRosa26 | | | | | | |
| -target | | | | | | |
| site | | | | | | |
| DNA | — | 5041 | MG29-1-hRosa26- | Nucleotide | N.A. | TTTCTTGAGTCAGAGT |
| sequence | | | target site-E4 | | | CTTGCT |
| of | | | | | | |
| hRosa26 | | | | | | |
| target | | | | | | |
| site | | | | | | |
| DNA | — | 5042 | MG29-1-hRosa26- | Nucleotide | N.A. | TTCTTGAGTCAGAGTC |
| sequence | | | target site-F4 | | | TTGCTC |
| of | | | | | | |
| hRosa26 | | | | | | |
| target | | | | | | |
| site | | | | | | |
| DNA | — | 5043 | MG29-1-hRosa26- | Nucleotide | N.A. | TCTTGAGTCAGAGTCT |
| sequence | | | target site-G4 | | | TGCTCC |
| of | | | | | | |
| hRosa26 | | | | | | |
| target | | | | | | |
| site | | | | | | |
| DNA | — | 5044 | MG29-1-hRosa26- | Nucleotide | N.A. | CTTGAGTCAGAGTCTT |
| sequence | | | target site-H4 | | | GCTCCG |
| of | | | | | | |
| hRosa26 | | | | | | |
| target | | | | | | |
| site | | | | | | |
| DNA | — | 5045 | MG29-1-hRosa26- | Nucleotide | N.A. | TTGAGTCAGAGTCTTG |
| sequence | | | target site-A5 | | | CTCCGT |
| of | | | | | | |
| hRosa26 | | | | | | |
| target | | | | | | |
| site | | | | | | |
| DNA | — | 5046 | MG29-1-hRosa26- | Nucleotide | N.A | TGTATTTTTAGTAGAG |
| sequence | | | target site-B5 | | | ATGGGG |
| of | | | | | | |
| hRosa26 | | | | | | |
| target | | | | | | |
| site | | | | | | |
| DNA | — | 5047 | MG29-1-hRosa26- | Nucleotide | N.A. | GTATTTTTAGTAGAGA |
| sequence | | | target site-C5 | | | TGGGGT |
| of | | | | | | |
| hRosa26 | | | | | | |
| target | | | | | | |
| site | | | | | | |
| DNA | — | 5048 | MG29-1-hRosa26- | Nucleotide | N.A. | TATTTTTAGTAGAGAT |
| sequence | | | target site-D5 | | | GGGGTT |
| of | | | | | | |
| hRosa26 | | | | | | |
| target | | | | | | |
| site | | | | | | |
| DNA | — | 5049 | MG29-1-hRosa26- | Nucleotide | N.A. | TAGTAGAGATGGGGTT |
| sequence | | | target site-E5 | | | TCACCA |
| of | | | | | | |
| hRosa26 | | | | | | |
| target | | | | | | |
| site | | | | | | |
| DNA | — | 5050 | MG29-1-hRosa26- | Nucleotide | N.A. | AGTAGAGATGGGGTTT |
| sequence | | | target site-F5 | | | CACCAC |
| of | | | | | | |
| hRosa26 | | | | | | |
| target | | | | | | |
| site | | | | | | |
| DNA | — | 5051 | MG29-1-hRosa26- | Nucleotide | N.A. | GTAGAGATGGGGTTTC |
| sequence | | | target site-G5 | | | ACCACG |
| of | | | | | | |
| hRosa26 | | | | | | |
| target | | | | | | |
| site | | | | | | |
| DNA | — | 5052 | MG29-1-hRosa26- | Nucleotide | N.A. | ACCACGTTGGTCAGGC |
| sequence | | | target site-H5 | | | TGGTCT |
| of | | | | | | |
| hRosa26 | | | | | | |
| target | | | | | | |
| site | | | | | | |
| DNA | — | 5053 | MG29-1-hRosa26- | Nucleotide | N.A. | ACCTTCCTGTGACTTC |
| sequence | | | target site-A6 | | | CTGGAG |
| of | | | | | | |
| hRosa26 | | | | | | |
| target | | | | | | |
| site | | | | | | |
| DNA | — | 5054 | MG29-1-hRosa26- | Nucleotide | N.A. | CCTTCCTGTGACTTCC |
| sequence | | | target site-B6 | | | TGGAGA |
| of | | | | | | |
| hRosa26 | | | | | | |
| target | | | | | | |
| site | | | | | | |
| DNA | — | 5055 | MG29-1-hRosa26- | Nucleotide | N.A. | TGGGGAAGGTTCTCTG |
| sequence | | | target site-C6 | | | TCTGCC |
| of | | | | | | |
| hRosa26 | | | | | | |
| target | | | | | | |
| site | | | | | | |
| MG29-1 | — | 5268 | MG29-1-FAS- | Nucleotide | N.A. | /AltR1/rUrArArUrUrUrCr |
| sgRNA | | | sgRNA-A1 | | | UrArCrUrGrUrUrGrUrAr |
| targeting | | | | | | GrArUrArGrGrUrArArA |
| FAS | | | | | | rCrArArCrCrGrArArCrU |
| | | | | | | rGrArUrGrA/AltR2/ |
| MG29-1 | — | 5269 | MG29-1-FAS- | Nucleotide | N.A. | /AltR1/rUrArArUrUrUrCr |
| sgRNA | | | sgRNA-B1 | | | UrArCrUrGrUrUrGrUrAr |
| targeting | | | | | | GrArUrCrCrUrGrArGrC |
| FAS | | | | | | rArArArGrArCrUrCrUrU |
| | | | | | | rGrCrUrArC/AltR2/ |
| MG29-1 | — | 5270 | MG29-1-FAS- | Nucleotide | N.A. | /AltR1/rUrArArUrUrUrCr |
| sgRNA | | | sgRNA-C1 | | | UrArCrUrGrUrUrGrUrAr |
| targeting | | | | | | GrArUrCrArGrArArCrG |
| FAS | | | | | | rUrGrGrCrArUrCrArArC |
| | | | | | | rArUrCrArC/AltR2/ |
| MG29-1 | — | 5271 | MG29-1-FAS- | Nucleotide | N.A. | /AltR1/rUrArArUrUrUrCr |
| sgRNA | | | sgRNA-D1 | | | UrArCrUrGrUrUrGrUrAr |
| targeting | | | | | | GrArUrUrCrArCrCrArGr |
| FAS | | | | | | CrUrCrCrCrArUrGrUrGr |
| | | | | | | ArUrGrUrU/AltR2/ |
| MG29-1 | — | 5272 | MG29-1-FAS- | Nucleotide | N.A. | /AltR1/rUrArArUrUrUrCr |
| sgRNA | | | sgRNA-E1 | | | UrArCrUrGrUrUrGrUrAr |
| targeting | | | | | | GrArUrUrGrUrGrUrCrU |
| FAS | | | | | | rArUrUrArGrArUrGrCrU |
| | | | | | | rCrArGrArG/AltR2/ |
| MG29-1 | — | 5273 | MG29-1-FAS- | Nucleotide | N.A. | /AltR1/rUrArArUrUrUrCr |
| sgRNA | | | sgRNA-F1 | | | UrArCrUrGrUrUrGrUrAr |
| targeting | | | | | | GrArUrGrUrGrUrCrUrA |
| FAS | | | | | | rUrUrArGrArUrGrCrUrC |
| | | | | | | rArGrArGrU/AltR2/ |
| MG29-1 | — | 5274 | MG29-1-FAS- | Nucleotide | N.A. | /AltR1/rUrArArUrUrUrCr |
| sgRNA | | | sgRNA-G1 | | | UrArCrUrGrUrUrGrUrAr |
| targeting | | | | | | GrArUrUrGrUrCrUrArUr |
| FAS | | | | | | UrArGrArUrGrCrUrCrAr |
| | | | | | | GrArGrUrG/AltR2/ |
| MG29-1 | — | 5275 | MG29-1-FAS- | Nucleotide | N.A. | /AltR1/rUrArArUrUrUrCr |
| sgRNA | | | sgRNA-H1 | | | UrArCrUrGrUrUrGrUrAr |
| targeting | | | | | | GrArUrGrCrArUrCrUrG |
| FAS | | | | | | rUrCrArCrUrGrCrArCrU |
| | | | | | | rUrArCrCrA/AltR2/ |
| MG29-1 | — | 5276 | MG29-1-FAS- | Nucleotide | N.A. | /AltR1/rUrArArUrUrUrCr |
| sgRNA | | | sgRNA-A2 | | | UrArCrUrGrUrUrGrUrAr |
| targeting | | | | | | GrArUrCrArUrCrUrGrUr |
| FAS | | | | | | CrArCrUrGrCrArCrUrUr |
| | | | | | | ArCrCrArC/AltR2/ |
| MG29-1 | — | 5277 | MG29-1-FAS- | Nucleotide | N.A. | /AltR1/rUrArArUrUrUrCr |
| sgRNA | | | sgRNA-B2 | | | UrArCrUrGrUrUrGrUrAr |
| targeting | | | | | | GrArUrArGrUrArGrArC |
| FAS | | | | | | rUrGrUrUrArGrUrGrCr |
| | | | | | | CrArUrGrArG/AltR2/ |
| MG29-1 | — | 5278 | MG29-1-FAS- | Nucleotide | N.A. | /AltR1/rUrArArUrUrUrCr |
| sgRNA | | | sgRNA-C2 | | | UrArCrUrGrUrUrGrUrAr |
| targeting | | | | | | GrArUrArUrUrUrUrArCr |
| FAS | | | | | | ArGrGrUrUrCrUrUrArCr |
| | | | | | | GrUrCrUrG/AltR2/ |
| MG29-1 | — | 5279 | MG29-1-FAS- | Nucleotide | N.A. | /AltR1/rUrArArUrUrUrCr |
| sgRNA | | | sgRNA-D2 | | | UrArCrUrGrUrUrGrUrAr |
| targeting | | | | | | GrArUrUrUrUrUrArCrAr |
| FAS | | | | | | GrGrUrUrCrUrUrArCrG |
| | | | | | | rUrCrUrGrU/AltR2/ |
| MG29-1 | — | 5280 | MG29-1-FAS- | Nucleotide | N.A. | /AltR1/rUrArArUrUrUrCr |
| sgRNA | | | sgRNA-E2 | | | UrArCrUrGrUrUrGrUrAr |
| targeting | | | | | | GrArUrArCrArGrGrUrU |
| FAS | | | | | | rCrUrUrArCrGrUrCrUrG |
| | | | | | | rUrUrGrCrU/AltR2/ |
| MG29-1 | — | 5281 | MG29-1-FAS- | Nucleotide | N.A. | /AltR1/rUrArArUrUrUrCr |
| sgRNA | | | sgRNA-F2 | | | UrArCrUrGrUrUrGrUrAr |
| targeting | | | | | | GrArUrCrArGrGrUrUrC |
| FAS | | | | | | rUrUrArCrGrUrCrUrGrU |
| | | | | | | rUrGrCrUrA/AltR2/ |
| MG29-1 | — | 5282 | MG29-1-FAS- | Nucleotide | N.A. | /AltR1/rUrArArUrUrUrCr |
| sgRNA | | | sgRNA-G2 | | | UrArCrUrGrUrUrGrUrAr |
| targeting | | | | | | GrArUrGrUrGrUrArArC |
| FAS | | | | | | rArUrArCrCrUrGrGrAr |
| | | | | | | GrGrArCrArG/AltR2/ |
| MG29-1 | — | 5283 | MG29-1-FAS- | Nucleotide | N.A. | /AltR1/rUrArArUrUrUrCr |
| sgRNA | | | sgRNA-H2 | | | UrArCrUrGrUrUrGrUrAr |
| targeting | | | | | | GrArUrUrGrUrArArCrAr |
| FAS | | | | | | UrArCrCrUrGrGrArGrG |
| | | | | | | rArCrArGrG/AltR2/ |
| MG29-1 | — | 5284 | MG29-1-FAS- | Nucleotide | N.A. | /AltR1/rUrArArUrUrUrCr |
| sgRNA | | | sgRNA-A3 | | | UrArCrUrGrUrUrGrUrAr |
| targeting | | | | | | GrArUrGrGrArCrGrArU |
| FAS | | | | | | rArArUrCrUrArGrCrArA |
| | | | | | | rCrArGrArC/AltR2/ |
| MG29-1 | — | 5285 | MG29-1-FAS- | Nucleotide | N.A. | /AltR1/rUrArArUrUrUrCr |
| sgRNA | | | sgRNA-B3 | | | UrArCrUrGrUrUrGrUrAr |
| targeting | | | | | | GrArUrGrArCrGrArUrA |
| FAS | | | | | | rArUrCrUrArGrCrArArC |
| | | | | | | rArGrArCrG/AltR2/ |
| MG29-1 | — | 5286 | MG29-1-FAS- | Nucleotide | N.A. | /AltR1/rUrArArUrUrUrCr |
| sgRNA | | | sgRNA-C3 | | | UrArCrUrGrUrUrGrUrAr |
| targeting | | | | | | GrArUrUrUrCrCrUrUrGr |
| FAS | | | | | | GrGrCrArGrGrUrGrArA |
| | | | | | | rArGrGrArA/AltR2/ |
| MG29-1 | — | 5287 | MG29-1-FAS- | Nucleotide | N.A. | /AltR1/rUrArArUrUrUrCr |
| sgRNA | | | sgRNA-D3 | | | UrArCrUrGrUrUrGrUrAr |
| targeting | | | | | | GrArUrUrCrCrUrUrGrG |
| FAS | | | | | | rGrCrArGrGrUrGrArAr |
| | | | | | | ArGrGrArArA/AltR2/ |
| MG29-1 | — | 5288 | MG29-1-FAS- | Nucleotide | N.A. | /AltR1/rUrArArUrUrUrCr |
| sgRNA | | | sgRNA-E3 | | | UrArCrUrGrUrUrGrUrAr |
| targeting | | | | | | GrArUrCrCrUrUrGrGrG |
| FAS | | | | | | rCrArGrGrUrGrArArAr |
| | | | | | | GrGrArArArG/AltR2/ |
| MG29-1 | — | 5289 | MG29-1-FAS- | Nucleotide | N.A. | /AltR1/rUrArArUrUrUrCr |
| sgRNA | | | sgRNA-F3 | | | UrArCrUrGrUrUrGrUrAr |
| targeting | | | | | | GrArUrCrUrUrGrGrGrC |
| FAS | | | | | | rArGrGrUrGrArArArGr |
| | | | | | | GrArArArGrC/AltR2/ |
| MG29-1 | — | 5290 | MG29-1-FAS- | Nucleotide | N.A. | /AltR1/rUrArArUrUrUrCr |
| sgRNA | | | sgRNA-G3 | | | UrArCrUrGrUrUrGrUrAr |
| targeting | | | | | | GrArUrUrCrUrUrCrCrAr |
| FAS | | | | | | ArArUrGrCrArGrArArG |
| | | | | | | rArUrGrUrA/AltR2/ |
| MG29-1 | — | 5291 | MG29-1-FAS- | Nucleotide | N.A. | /AltR1/rUrArArUrUrUrCr |
| sgRNA | | | sgRNA-H3 | | | UrArCrUrGrUrUrGrUrAr |
| targeting | | | | | | GrArUrCrUrUrCrCrArAr |
| FAS | | | | | | ArUrGrCrArGrArArGrA |
| | | | | | | rUrGrUrArG/AltR2/ |
| MG29-1 | — | 5292 | MG29-1-FAS- | Nucleotide | N.A. | /AltR1/rUrArArUrUrUrCr |
| sgRNA | | | sgRNA-A4 | | | UrArCrUrGrUrUrGrUrAr |
| targeting | | | | | | GrArUrUrUrCrCrArArAr |
| FAS | | | | | | UrGrCrArGrArArGrArU |
| | | | | | | rGrUrArGrA/AltR2/ |
| MG29-1 | — | 5293 | MG29-1-FAS- | Nucleotide | N.A. | /AltR1/rUrArArUrUrUrCr |
| sgRNA | | | sgRNA-B4 | | | UrArCrUrGrUrUrGrUrAr |
| targeting | | | | | | GrArUrArArGrArCrUrCr |
| FAS | | | | | | UrUrArCrCrArUrGrUrCr |
| | | | | | | CrUrUrCrA/AltR2/ |
| MG29-1 | — | 5294 | MG29-1-FAS- | Nucleotide | N.A. | /AltR1/rUrArArUrUrUrCr |
| sgRNA | | | sgRNA-C4 | | | UrArCrUrGrUrUrGrUrAr |
| targeting | | | | | | GrArUrArGrArCrUrCrUr |
| FAS | | | | | | UrArCrCrArUrGrUrCrCr |
| | | | | | | UrUrCrArU/AltR2/ |
| MG29-1 | — | 5295 | MG29-1-FAS- | Nucleotide | N.A. | /AltR1/rUrArArUrUrUrCr |
| sgRNA | | | sgRNA-D4 | | | UrArCrUrGrUrUrGrUrAr |
| targeting | | | | | | GrArUrGrArArGrArArA |
| FAS | | | | | | rArArUrGrGrGrCrUrUr |
| | | | | | | UrGrUrCrUrG/AltR2/ |
| MG29-1 | — | 5296 | MG29-1-FAS- | Nucleotide | N.A. | /AltR1/rUrArArUrUrUrCr |
| sgRNA | | | sgRNA-E4 | | | UrArCrUrGrUrUrGrUrAr |
| targeting | | | | | | GrArUrUrCrUrGrUrGrU |
| FAS | | | | | | rArCrUrCrCrUrUrCrCrC |
| | | | | | | rUrUrCrUrU/AltR2/ |
| MG29-1 | — | 5297 | MG29-1-FAS- | Nucleotide | N.A. | /AltR1/rUrArArUrUrUrCr |
| sgRNA | | | sgRNA-F4 | | | UrArCrUrGrUrUrGrUrAr |
| targeting | | | | | | GrArUrCrArArArCrUrGr |
| FAS | | | | | | ArUrUrUrUrCrUrArGrGr |
| | | | | | | CrUrUrArG/AltR2/ |
| MG29-1 | — | 5298 | MG29-1-FAS- | Nucleotide | N.A. | /AltR1/rUrArArUrUrUrCr |
| sgRNA | | | sgRNA-G4 | | | UrArCrUrGrUrUrGrUrAr |
| targeting | | | | | | GrArUrCrUrArGrGrCrU |
| FAS | | | | | | rUrArGrArArGrUrGrGr |
| | | | | | | ArArArUrArA/AltR2/ |
| MG29-1 | — | 5299 | MG29-1-FAS- | Nucleotide | N.A. | /AltR1/rUrArArUrUrUrCr |
| sgRNA | | | sgRNA-H4 | | | UrArCrUrGrUrUrGrUrAr |
| targeting | | | | | | GrArUrUrArGrGrCrUrU |
| FAS | | | | | | rArGrArArGrUrGrGrAr |
| | | | | | | ArArUrArArA/AltR2/ |
| MG29-1 | — | 5300 | MG29-1-FAS- | Nucleotide | N.A. | /AltR1/rUrArArUrUrUrCr |
| sgRNA | | | sgRNA-A5 | | | UrArCrUrGrUrUrGrUrAr |
| targeting | | | | | | GrArUrUrUrUrGrUrArAr |
| FAS | | | | | | CrUrCrUrArCrUrGrUrAr |
| | | | | | | UrGrUrGrA/AltR2/ |
| MG29-1 | — | 5301 | MG29-1-FAS- | Nucleotide | N.A. | /AltR1/rUrArArUrUrUrCr |
| sgRNA | | | sgRNA-B5 | | | UrArCrUrGrUrUrGrUrAr |
| targeting | | | | | | GrArUrUrUrGrUrArArCr |
| FAS | | | | | | UrCrUrArCrUrGrUrArUr |
| | | | | | | GrUrGrArA/AltR2/ |
| MG29-1 | — | 5302 | MG29-1-FAS- | Nucleotide | N.A. | /AltR1/rUrArArUrUrUrCr |
| sgRNA | | | sgRNA-C5 | | | UrArCrUrGrUrUrGrUrAr |
| targeting | | | | | | GrArUrUrGrUrArArCrUr |
| FAS | | | | | | CrUrArCrUrGrUrArUrGr |
| | | | | | | UrGrArArC/AltR2/ |
| MG29-1 | — | 5303 | MG29-1-FAS- | Nucleotide | N.A. | /AltR1/rUrArArUrUrUrCr |
| sgRNA | | | sgRNA-D5 | | | UrArCrUrGrUrUrGrUrAr |
| targeting | | | | | | GrArUrGrUrArArCrUrCr |
| FAS | | | | | | UrArCrUrGrUrArUrGrUr |
| | | | | | | GrArArCrA/AltR2/ |
| MG29-1 | — | 5304 | MG29-1-FAS- | Nucleotide | N.A. | /AltR1/rUrArArUrUrUrCr |
| sgRNA | | | sgRNA-E5 | | | UrArCrUrGrUrUrGrUrAr |
| targeting | | | | | | GrArUrUrArArCrUrCrUr |
| FAS | | | | | | ArCrUrGrUrArUrGrUrG |
| | | | | | | rArArCrArC/AltR2/ |
| MG29-1 | — | 5305 | MG29-1-FAS- | Nucleotide | N.A. | /AltR1/rUrArArUrUrUrCr |
| sgRNA | | | sgRNA-F5 | | | UrArCrUrGrUrUrGrUrAr |
| targeting | | | | | | GrArUrGrUrUrUrArCrAr |
| FAS | | | | | | UrCrUrGrCrArCrUrUrGr |
| | | | | | | GrUrArUrU/AltR2/ |
| MG29-1 | — | 5306 | MG29-1-FAS- | Nucleotide | N.A. | /AltR1/rUrArArUrUrUrCr |
| sgRNA | | | sgRNA-G5 | | | UrArCrUrGrUrUrGrUrAr |
| targeting | | | | | | GrArUrCrArUrCrUrGrCr |
| FAS | | | | | | ArCrUrUrGrGrUrArUrUr |
| | | | | | | CrUrGrGrG/AltR2/ |
| MG29-1 | — | 5307 | MG29-1-FAS- | Nucleotide | N.A. | /AltR1/rUrArArUrUrUrCr |
| sgRNA | | | sgRNA-H5 | | | UrArCrUrGrUrUrGrUrAr |
| targeting | | | | | | GrArUrUrCrCrUrGrUrAr |
| FAS | | | | | | UrUrUrUrUrUrUrUrUrCr |
| | | | | | | UrArGrArU/AltR2/ |
| MG29-1 | — | 5308 | MG29-1-FAS- | Nucleotide | N.A. | /AltR1/rUrArArUrUrUrCr |
| sgRNA | | | sgRNA-A6 | | | UrArCrUrGrUrUrGrUrAr |
| targeting | | | | | | GrArUrUrUrUrUrUrCrUr |
| FAS | | | | | | ArGrArUrGrUrGrArArC |
| | | | | | | rArUrGrGrA/AltR2/ |
| MG29-1 | — | 5309 | MG29-1-FAS- | Nucleotide | N.A. | /AltR1/rUrArArUrUrUrCr |
| sgRNA | | | sgRNA-B6 | | | UrArCrUrGrUrUrGrUrAr |
| targeting | | | | | | GrArUrUrUrUrUrCrUrAr |
| FAS | | | | | | GrArUrGrUrGrArArCrA |
| | | | | | | rUrGrGrArA/AltR2/ |
| MG29-1 | — | 5310 | MG29-1-FAS- | Nucleotide | N.A. | /AltR1/rUrArArUrUrUrCr |
| sgRNA | | | sgRNA-C6 | | | UrArCrUrGrUrUrGrUrAr |
| targeting | | | | | | GrArUrUrUrUrCrUrArGr |
| FAS | | | | | | ArUrGrUrGrArArCrArUr |
| | | | | | | GrGrArArU/AltR2/ |
| MG29-1 | — | 5311 | MG29-1-FAS- | Nucleotide | N.A. | /AltR1/rUrArArUrUrUrCr |
| sgRNA | | | sgRNA-D6 | | | UrArCrUrGrUrUrGrUrAr |
| targeting | | | | | | GrArUrUrUrCrUrArGrAr |
| FAS | | | | | | UrGrUrGrArArCrArUrG |
| | | | | | | rGrArArUrC/AltR2/ |
| MG29-1 | — | 5312 | MG29-1-FAS- | Nucleotide | N.A. | /AltR1/rUrArArUrUrUrCr |
| sgRNA | | | sgRNA-E6 | | | UrArCrUrGrUrUrGrUrAr |
| targeting | | | | | | GrArUrUrCrUrArGrArUr |
| FAS | | | | | | GrUrGrArArCrArUrGrG |
| | | | | | | rArArUrCrA/AltR2/ |
| MG29-1 | — | 5313 | MG29-1-FAS- | Nucleotide | N.A. | /AltR1/rUrArArUrUrUrCr |
| sgRNA | | | sgRNA-F6 | | | UrArCrUrGrUrUrGrUrAr |
| targeting | | | | | | GrArUrCrUrArGrArUrG |
| FAS | | | | | | rUrGrArArCrArUrGrGr |
| | | | | | | ArArUrCrArU/AltR2/ |
| MG29-1 | — | 5314 | MG29-1-FAS- | Nucleotide | N.A. | /AltR1/rUrArArUrUrUrCr |
| sgRNA | | | sgRNA-G6 | | | UrArCrUrGrUrUrGrUrAr |
| targeting | | | | | | GrArUrUrArGrArUrGrU |
| FAS | | | | | | rGrArArCrArUrGrGrAr |
| | | | | | | ArUrCrArUrC/AltR2/ |
| MG29-1 | — | 5315 | MG29-1-FAS- | Nucleotide | N.A. | /AltR1/rUrArArUrUrUrCr |
| sgRNA | | | sgRNA-H6 | | | UrArCrUrGrUrUrGrUrAr |
| targeting | | | | | | GrArUrCrArCrUrUrGrG |
| FAS | | | | | | rUrGrUrUrGrCrUrGrGr |
| | | | | | | UrGrArGrUrG/AltR2/ |
| MG29-1 | — | 5316 | MG29-1-FAS- | Nucleotide | N.A. | /AltR1/rUrArArUrUrUrCr |
| sgRNA | | | sgRNA-A7 | | | UrArCrUrGrUrUrGrUrAr |
| targeting | | | | | | GrArUrUrCrUrUrCrUrUr |
| FAS | | | | | | CrUrUrUrUrGrCrCrArAr |
| | | | | | | UrUrCrCrA/AltR2/ |
| MG29-1 | — | 5317 | MG29-1-FAS- | Nucleotide | N.A. | /AltR1/rUrArArUrUrUrCr |
| sgRNA | | | sgRNA-B7 | | | UrArCrUrGrUrUrGrUrAr |
| targeting | | | | | | GrArUrGrCrCrArArUrUr |
| FAS | | | | | | CrCrArCrUrArArUrUrGr |
| | | | | | | UrUrUrGrG/AltR2/ |
| MG29-1 | — | 5318 | MG29-1-FAS- | Nucleotide | N.A. | /AltR1/rUrArArUrUrUrCr |
| sgRNA | | | sgRNA-C7 | | | UrArCrUrGrUrUrGrUrAr |
| targeting | | | | | | GrArUrCrCrArArUrUrCr |
| FAS | | | | | | CrArCrUrArArUrUrGrUr |
| | | | | | | UrUrGrGrG/AltR2/ |
| MG29-1 | — | 5319 | MG29-1-FAS- | Nucleotide | N.A. | /AltR1/rUrArArUrUrUrCr |
| sgRNA | | | sgRNA-D7 | | | UrArCrUrGrUrUrGrUrAr |
| targeting | | | | | | GrArUrArArCrArArArGr |
| FAS | | | | | | CrArArGrArArCrUrUrAr |
| | | | | | | CrCrCrCrA/AltR2/ |
| MG29-1 | — | 5320 | MG29-1-FAS- | Nucleotide | N.A. | /AltR1/rUrArArUrUrUrCr |
| sgRNA | | | sgRNA-E7 | | | UrArCrUrGrUrUrGrUrAr |
| targeting | | | | | | GrArUrUrUrUrGrUrUrCr |
| FAS | | | | | | UrUrUrCrArGrUrGrArAr |
| | | | | | | GrArGrArA/AltR2/ |
| MG29-1 | — | 5321 | MG29-1-FAS- | Nucleotide | N.A. | /AltR1/rUrArArUrUrUrCr |
| sgRNA | | | sgRNA-F7 | | | UrArCrUrGrUrUrGrUrAr |
| targeting | | | | | | GrArUrUrUrCrUrUrUrCr |
| FAS | | | | | | ArGrUrGrArArGrArGrA |
| | | | | | | rArArGrGrA/AltR2/ |
| MG29-1 | — | 5322 | MG29-1-FAS- | Nucleotide | N.A. | /AltR1/rUrArArUrUrUrCr |
| sgRNA | | | sgRNA-G7 | | | UrArCrUrGrUrUrGrUrAr |
| targeting | | | | | | GrArUrArGrUrGrArArG |
| FAS | | | | | | rArGrArArArGrGrArAr |
| | | | | | | GrUrArCrArG/AltR2/ |
| MG29-1 | — | 5323 | MG29-1-FAS- | Nucleotide | N.A. | /AltR1/rUrArArUrUrUrCr |
| sgRNA | | | sgRNA-H7 | | | UrArCrUrGrUrUrGrUrAr |
| targeting | | | | | | GrArUrArArUrArCrCrUr |
| FAS | | | | | | ArCrArGrGrArUrUrUrAr |
| | | | | | | ArArGrUrU/AltR2/ |
| MG29-1 | — | 5324 | MG29-1-FAS- | Nucleotide | N.A. | /AltR1/rUrArArUrUrUrCr |
| sgRNA | | | sgRNA-A8 | | | UrArCrUrGrUrUrGrUrAr |
| targeting | | | | | | GrArUrArArGrUrUrGrG |
| FAS | | | | | | rArGrArUrUrCrArUrGrA |
| | | | | | | rGrArArCrC/AltR2/ |
| MG29-1 | — | 5325 | MG29-1-FAS- | Nucleotide | N.A. | /AltR1/rUrArArUrUrUrCr |
| sgRNA | | | sgRNA-B8 | | | UrArCrUrGrUrUrGrUrAr |
| targeting | | | | | | GrArUrCrCrUrUrUrCrUr |
| FAS | | | | | | GrUrGrCrUrUrUrCrUrG |
| | | | | | | rCrArUrGrU/AltR2/ |
| MG29-1 | — | 5326 | MG29-1-FAS- | Nucleotide | N.A. | /AltR1/rUrArArUrUrUrCr |
| sgRNA | | | sgRNA-C8 | | | UrArCrUrGrUrUrGrUrAr |
| targeting | | | | | | GrArUrCrUrUrUrCrUrGr |
| FAS | | | | | | UrGrCrUrUrUrCrUrGrCr |
| | | | | | | ArUrGrUrU/AltR2/ |
| MG29-1 | — | 5327 | MG29-1-FAS- | Nucleotide | N.A. | /AltR1/rUrArArUrUrUrCr |
| sgRNA | | | sgRNA-D8 | | | UrArCrUrGrUrUrGrUrAr |
| targeting | | | | | | GrArUrUrGrUrGrCrUrU |
| FAS | | | | | | rUrCrUrGrCrArUrGrUrU |
| | | | | | | rUrUrCrUrG/AltR2/ |
| MG29-1 | — | 5328 | MG29-1-FAS- | Nucleotide | N.A. | /AltR1/rUrArArUrUrUrCr |
| sgRNA | | | sgRNA-E8 | | | UrArCrUrGrUrUrGrUrAr |
| targeting | | | | | | GrArUrUrGrCrArUrGrU |
| FAS | | | | | | rUrUrUrCrUrGrUrArCrU |
| | | | | | | rUrCrCrUrU/AltR2/ |
| MG29-1 | — | 5329 | MG29-1-FAS- | Nucleotide | N.A. | /AltR1/rUrArArUrUrUrCr |
| sgRNA | | | sgRNA-F8 | | | UrArCrUrGrUrUrGrUrAr |
| targeting | | | | | | GrArUrCrUrGrUrArCrUr |
| FAS | | | | | | UrCrCrUrUrUrCrUrCrUr |
| | | | | | | UrCrArCrU/AltR2/ |
| MG29-1 | — | 5330 | MG29-1-FAS- | Nucleotide | N.A. | /AltR1/rUrArArUrUrUrCr |
| sgRNA | | | sgRNA-G8 | | | UrArCrUrGrUrUrGrUrAr |
| targeting | | | | | | GrArUrUrUrGrCrUrUrUr |
| FAS | | | | | | CrUrArGrGrArArArCrAr |
| | | | | | | GrUrGrGrC/AltR2/ |
| MG29-1 | — | 5331 | MG29-1-FAS- | Nucleotide | N.A. | /AltR1/rUrArArUrUrUrCr |
| sgRNA | | | sgRNA-H8 | | | UrArCrUrGrUrUrGrUrAr |
| targeting | | | | | | GrArUrUrGrCrUrUrUrCr |
| FAS | | | | | | UrArGrGrArArArCrArG |
| | | | | | | rUrGrGrCrA/AltR2/ |
| MG29-1 | — | 5332 | MG29-1-FAS- | Nucleotide | N.A. | /AltR1/rUrArArUrUrUrCr |
| sgRNA | | | sgRNA-A9 | | | UrArCrUrGrUrUrGrUrAr |
| targeting | | | | | | GrArUrGrCrUrUrUrCrUr |
| FAS | | | | | | ArGrGrArArArCrArGrU |
| | | | | | | rGrGrCrArA/AltR2/ |
| MG29-1 | — | 5333 | MG29-1-FAS- | Nucleotide | N.A. | /AltR1/rUrArArUrUrUrCr |
| sgRNA | | | sgRNA-B9 | | | UrArCrUrGrUrUrGrUrAr |
| targeting | | | | | | GrArUrCrUrUrUrCrUrAr |
| FAS | | | | | | GrGrArArArCrArGrUrG |
| | | | | | | rGrCrArArU/AltR2/ |
| MG29-1 | — | 5334 | MG29-1-FAS- | Nucleotide | N.A. | /AltR1/rUrArArUrUrUrCr |
| sgRNA | | | sgRNA-C9 | | | UrArCrUrGrUrUrGrUrAr |
| targeting | | | | | | GrArUrUrArGrGrArArA |
| FAS | | | | | | rCrArGrUrGrGrCrArAr |
| | | | | | | UrArArArUrU/AltR2/ |
| MG29-1 | — | 5335 | MG29-1-FAS- | Nucleotide | N.A. | /AltR1/rUrArArUrUrUrCr |
| sgRNA | | | sgRNA-D9 | | | UrArCrUrGrUrUrGrUrAr |
| targeting | | | | | | GrArUrArGrArCrUrArUr |
| FAS | | | | | | UrUrUrCrUrArUrUrUrUr |
| | | | | | | UrCrArGrA/AltR2/ |
| MG29-1 | — | 5336 | MG29-1-FAS- | Nucleotide | N.A. | /AltR1/rUrArArUrUrUrCr |
| sgRNA | | | sgRNA-E9 | | | UrArCrUrGrUrUrGrUrAr |
| targeting | | | | | | GrArUrCrUrArUrUrUrUr |
| FAS | | | | | | UrCrArGrArUrGrUrUrG |
| | | | | | | rArCrUrUrG/AltR2/ |
| MG29-1 | — | 5337 | MG29-1-FAS- | Nucleotide | N.A. | /AltR1/rUrArArUrUrUrCr |
| sgRNA | | | sgRNA-F9 | | | UrArCrUrGrUrUrGrUrAr |
| targeting | | | | | | GrArUrUrArUrUrUrUrUr |
| FAS | | | | | | CrArGrArUrGrUrUrGrA |
| | | | | | | rCrUrUrGrA/AltR2/ |
| MG29-1 | — | 5338 | MG29-1-FAS- | Nucleotide | N.A. | /AltR1/rUrArArUrUrUrCr |
| sgRNA | | | sgRNA-G9 | | | UrArCrUrGrUrUrGrUrAr |
| targeting | | | | | | GrArUrUrCrArGrArUrG |
| FAS | | | | | | rUrUrGrArCrUrUrGrAr |
| | | | | | | GrUrArArArU/AltR2/ |
| MG29-1 | — | 5339 | MG29-1-FAS- | Nucleotide | N.A. | /AltR1/rUrArArUrUrUrCr |
| sgRNA | | | sgRNA-H9 | | | UrArCrUrGrUrUrGrUrAr |
| targeting | | | | | | GrArUrCrArGrArUrGrU |
| FAS | | | | | | rUrGrArCrUrUrGrArGr |
| | | | | | | UrArArArUrA/AltR2/ |
| MG29-1 | — | 5340 | MG29-1-FAS- | Nucleotide | N.A. | /AltR1/rUrArArUrUrUrCr |
| sgRNA | | | sgRNA-A10 | | | UrArCrUrGrUrUrGrUrAr |
| targeting | | | | | | GrArUrArGrArUrGrUrU |
| FAS | | | | | | rGrArCrUrUrGrArGrUr |
| | | | | | | ArArArUrArU/AltR2/ |
| MG29-1 | — | 5341 | MG29-1-FAS- | Nucleotide | N.A. | /AltR1/rUrArArUrUrUrCr |
| sgRNA | | | sgRNA-B10 | | | UrArCrUrGrUrUrGrUrAr |
| targeting | | | | | | GrArUrUrUrCrGrArArAr |
| FAS | | | | | | GrArArUrGrGrUrGrUrC |
| | | | | | | rArArUrGrA/AltR2/ |
| MG29-1 | — | 5342 | MG29-1-FAS- | Nucleotide | N.A. | /AltR1/rUrArArUrUrUrCr |
| sgRNA | | | sgRNA-C10 | | | UrArCrUrGrUrUrGrUrAr |
| targeting | | | | | | GrArUrUrArCrUrCrUrUr |
| FAS | | | | | | GrCrArGrArGrArArArA |
| | | | | | | rUrUrCrArG/AltR2/ |
| MG29-1 | — | 5343 | MG29-1-FAS- | Nucleotide | N.A. | /AltR1/rUrArArUrUrUrCr |
| sgRNA | | | sgRNA-D10 | | | UrArCrUrGrUrUrGrUrAr |
| targeting | | | | | | GrArUrUrUrGrUrUrUrUr |
| FAS | | | | | | UrCrArCrUrCrUrArGrAr |
| | | | | | | CrCrArArG/AltR2/ |
| MG29-1 | — | 5344 | MG29-1-FAS- | Nucleotide | N.A. | /AltR1/rUrArArUrUrUrCr |
| sgRNA | | | sgRNA-E10 | | | UrArCrUrGrUrUrGrUrAr |
| targeting | | | | | | GrArUrUrCrArCrUrCrUr |
| FAS | | | | | | ArGrArCrCrArArGrCrUr |
| | | | | | | UrUrGrGrA/AltR2/ |
| MG29-1 | — | 5345 | MG29-1-FAS- | Nucleotide | N.A. | /AltR1/rUrArArUrUrUrCr |
| sgRNA | | | sgRNA-F10 | | | UrArCrUrGrUrUrGrUrAr |
| targeting | | | | | | GrArUrCrArCrUrCrUrAr |
| FAS | | | | | | GrArCrCrArArGrCrUrUr |
| | | | | | | UrGrGrArU/AltR2/ |
| MG29-1 | — | 5346 | MG29-1-FAS- | Nucleotide | N.A. | /AltR1/rUrArArUrUrUrCr |
| sgRNA | | | sgRNA-G10 | | | UrArCrUrGrUrUrGrUrAr |
| targeting | | | | | | GrArUrArCrUrCrUrArGr |
| FAS | | | | | | ArCrCrArArGrCrUrUrUr |
| | | | | | | GrGrArUrU/AltR2/ |
| MG29-1 | — | 5347 | MG29-1-FAS- | Nucleotide | N.A. | /AltR1/rUrArArUrUrUrCr |
| sgRNA | | | sgRNA-H10 | | | UrArCrUrGrUrUrGrUrAr |
| targeting | | | | | | GrArUrGrArUrUrUrCrAr |
| FAS | | | | | | UrUrUrCrUrGrArArGrUr |
| | | | | | | UrUrGrArA/AltR2/ |
| MG29-1 | — | 5348 | MG29-1-FAS- | Nucleotide | N.A. | /AltR1/rUrArArUrUrUrCr |
| sgRNA | | | sgRNA-A11 | | | UrArCrUrGrUrUrGrUrAr |
| targeting | | | | | | GrArUrArUrUrUrCrUrGr |
| FAS | | | | | | ArArGrUrUrUrGrArArUr |
| | | | | | | UrUrUrCrU/AltR2/ |
| MG29-1 | — | 5349 | MG29-1-FAS- | Nucleotide | N.A. | /AltR1/rUrArArUrUrUrCr |
| sgRNA | | | sgRNA-B11 | | | UrArCrUrGrUrUrGrUrAr |
| targeting | | | | | | GrArUrUrGrArArGrUrU |
| FAS | | | | | | rUrGrArArUrUrUrUrCrU |
| | | | | | | rGrArGrUrC/AltR2/ |
| MG29-1 | — | 5350 | MG29-1-FAS- | Nucleotide | N.A. | /AltR1/rUrArArUrUrUrCr |
| sgRNA | | | sgRNA-C11 | | | UrArCrUrGrUrUrGrUrAr |
| targeting | | | | | | GrArUrArArUrUrUrUrCr |
| FAS | | | | | | UrGrArGrUrCrArCrUrAr |
| | | | | | | GrUrArArU/AltR2/ |
| MG29-1 | — | 5351 | MG29-1-FAS- | Nucleotide | N.A. | /AltR1/rUrArArUrUrUrCr |
| sgRNA | | | sgRNA-D11 | | | UrArCrUrGrUrUrGrUrAr |
| targeting | | | | | | GrArUrCrUrGrArGrUrC |
| FAS | | | | | | rArCrUrArGrUrArArUrG |
| | | | | | | rUrCrCrUrU/AltR2/ |
| MG29-1 | — | 5352 | MG29-1-FAS- | Nucleotide | N.A. | /AltR1/rUrArArUrUrUrCr |
| sgRNA | | | sgRNA-E11 | | | UrArCrUrGrUrUrGrUrAr |
| targeting | | | | | | GrArUrUrGrArGrUrCrA |
| FAS | | | | | | rCrUrArGrUrArArUrGrU |
| | | | | | | rCrCrUrUrG/AltR2/ |
| MG29-1 | — | 5353 | MG29-1-FAS- | Nucleotide | N.A. | /AltR1/rUrArArUrUrUrCr |
| sgRNA | | | sgRNA-F11 | | | UrArCrUrGrUrUrGrUrAr |
| targeting | | | | | | GrArUrCrUrCrUrGrCrAr |
| FAS | | | | | | ArGrArGrUrArCrArArAr |
| | | | | | | GrArUrUrG/AltR2/ |
| MG29-1 | — | 5354 | MG29-1-FAS- | Nucleotide | N.A. | /AltR1/rUrArArUrUrUrCr |
| sgRNA | | | sgRNA-G11 | | | UrArCrUrGrUrUrGrUrAr |
| targeting | | | | | | GrArUrUrCrUrGrCrArAr |
| FAS | | | | | | GrArGrUrArCrArArArG |
| | | | | | | rArUrUrGrG/AltR2/ |
| MG29-1 | — | 5355 | MG29-1-FAS- | Nucleotide | N.A. | /AltR1/rUrArArUrUrUrCr |
| sgRNA | | | sgRNA-H11 | | | UrArCrUrGrUrUrGrUrAr |
| targeting | | | | | | GrArUrUrUrGrArGrArU |
| FAS | | | | | | rCrUrUrUrArArUrCrArA |
| | | | | | | rUrGrUrGrU/AltR2/ |
| MG29-1 | — | 5356 | MG29-1-FAS- | Nucleotide | N.A. | /AltR1/rUrArArUrUrUrCr |
| sgRNA | | | sgRNA-A12 | | | UrArCrUrGrUrUrGrUrAr |
| targeting | | | | | | GrArUrUrGrArGrArUrC |
| FAS | | | | | | rUrUrUrArArUrCrArArU |
| | | | | | | rGrUrGrUrC/AltR2/ |
| MG29-1 | — | 5357 | MG29-1-FAS- | Nucleotide | N.A. | /AltR1/rUrArArUrUrUrCr |
| sgRNA | | | sgRNA-B12 | | | UrArCrUrGrUrUrGrUrAr |
| targeting | | | | | | GrArUrGrArGrArUrCrU |
| FAS | | | | | | rUrUrArArUrCrArArUrG |
| | | | | | | rUrGrUrCrA/AltR2/ |
| MG29-1 | — | 5358 | MG29-1-FAS- | Nucleotide | N.A. | /AltR1/rUrArArUrUrUrCr |
| sgRNA | | | sgRNA-C12 | | | UrArCrUrGrUrUrGrUrAr |
| targeting | | | | | | GrArUrArGrArUrCrUrUr |
| FAS | | | | | | UrArArUrCrArArUrGrUr |
| | | | | | | GrUrCrArU/AltR2/ |
| MG29-1 | — | 5359 | MG29-1-FAS- | Nucleotide | N.A. | /AltR1/rUrArArUrUrUrCr |
| sgRNA | | | sgRNA-D12 | | | UrArCrUrGrUrUrGrUrAr |
| targeting | | | | | | GrArUrArUrCrArArUrGr |
| FAS | | | | | | UrGrUrCrArUrArCrGrCr |
| | | | | | | UrUrCrUrU/AltR2/ |
| MG29-1 | — | 5360 | MG29-1-FAS- | Nucleotide | N.A. | /AltR1/rUrArArUrUrUrCr |
| sgRNA | | | sgRNA-E12 | | | UrArCrUrGrUrUrGrUrAr |
| targeting | | | | | | GrArUrUrUrUrCrCrArUr |
| FAS | | | | | | GrArArGrUrUrGrArUrG |
| | | | | | | rCrCrArArU/AltR2/ |
| MG29-1 | — | 5361 | MG29-1-FAS- | Nucleotide | N.A. | /AltR1/rUrArArUrUrUrCr |
| sgRNA | | | sgRNA-F12 | | | UrArCrUrGrUrUrGrUrAr |
| targeting | | | | | | GrArUrCrArUrGrArArG |
| FAS | | | | | | rUrUrGrArUrGrCrCrArA |
| | | | | | | rUrUrArCrG/AltR2/ |
| MG29-1 | — | 5362 | MG29-1-FAS- | Nucleotide | N.A. | /AltR1/rUrArArUrUrUrCr |
| sgRNA | | | sgRNA-G12 | | | UrArCrUrGrUrUrGrUrAr |
| targeting | | | | | | GrArUrUrGrUrUrCrUrG |
| FAS | | | | | | rCrUrGrUrGrUrCrUrUr |
| | | | | | | GrGrArCrArU/AltR2/ |
| MG29-1 | — | 5363 | MG29-1-FAS- | Nucleotide | N.A. | /AltR1/rUrArArUrUrUrCr |
| sgRNA | | | sgRNA-H12 | | | UrArCrUrGrUrUrGrUrAr |
| targeting | | | | | | GrArUrGrGrCrUrUrCrA |
| FAS | | | | | | rUrUrGrArCrArCrCrArU |
| | | | | | | rUrCrUrUrU/AltR2/ |
| MG29-1 | — | 5364 | MG29-1-FAS- | Nucleotide | N.A. | /AltR1/rUrArArUrUrUrCr |
| sgRNA | | | sgRNA-A13 | | | UrArCrUrGrUrUrGrUrAr |
| targeting | | | | | | GrArUrGrCrUrUrCrArUr |
| FAS | | | | | | UrGrArCrArCrCrArUrUr |
| | | | | | | CrUrUrUrC/AltR2/ |
| MG29-1 | — | 5365 | MG29-1-FAS- | Nucleotide | N.A. | /AltR1/rUrArArUrUrUrCr |
| sgRNA | | | sgRNA-B13 | | | UrArCrUrGrUrUrGrUrAr |
| targeting | | | | | | GrArUrGrArArCrArArAr |
| FAS | | | | | | GrCrCrUrUrUrArArCrUr |
| | | | | | | UrGrArCrU/AltR2/ |
| MG29-1 | — | 5366 | MG29-1-FAS- | Nucleotide | N.A. | /AltR1/rUrArArUrUrUrCr |
| sgRNA | | | sgRNA-C13 | | | UrArCrUrGrUrUrGrUrAr |
| targeting | | | | | | GrArUrArCrUrUrGrArCr |
| FAS | | | | | | UrUrArGrUrGrUrCrArUr |
| | | | | | | GrArCrUrC/AltR2/ |
| DNA | — | 5367 | MG29-1-FAS- | Nucleotide | N.A. | AGGTAAACAACCGAAC |
| sequence | | | target site-A1 | | | TGATGA |
| of FAS | | | | | | |
| target | | | | | | |
| site | | | | | | |
| DNA | — | 5368 | MG29-1-FAS- | Nucleotide | N.A. | CCTGAGCAAAGACTCT |
| sequence | | | target site-B1 | | | TGCTAC |
| of FAS | | | | | | |
| target | | | | | | |
| site | | | | | | |
| DNA | — | 5369 | MG29-1-FAS- | Nucleotide | N.A. | CAGAACGTGGCATCAA |
| sequence | | | target site-C1 | | | CATCAC |
| of FAS | | | | | | |
| target | | | | | | |
| site | | | | | | |
| DNA | — | 5370 | MG29-1-FAS- | Nucleotide | N.A. | TCACCAGCTCCCATGT |
| sequence | | | target site-D1 | | | GATGTT |
| of FAS | | | | | | |
| target | | | | | | |
| site | | | | | | |
| DNA | — | 5371 | MG29-1-FAS- | Nucleotide | N.A. | TGTGTCTATTAGATGC |
| sequence | | | target site-E1 | | | TCAGAG |
| of FAS | | | | | | |
| target | | | | | | |
| site | | | | | | |
| DNA | — | 5372 | MG29-1-FAS- | Nucleotide | N.A. | GTGTCTATTAGATGCT |
| sequence | | | target site-F1 | | | CAGAGT |
| of FAS | | | | | | |
| target | | | | | | |
| site | | | | | | |
| DNA | — | 5373 | MG29-1-FAS- | Nucleotide | N.A. | TGTCTATTAGATGCTC |
| sequence | | | target site-G1 | | | AGAGTG |
| of FAS | | | | | | |
| target | | | | | | |
| site | | | | | | |
| DNA | — | 5374 | MG29-1-FAS- | Nucleotide | N.A. | GCATCTGTCACTGCAC |
| sequence | | | target site-H1 | | | TTACCA |
| of FAS | | | | | | |
| target | | | | | | |
| site | | | | | | |
| DNA | — | 5375 | MG29-1-FAS- | Nucleotide | N.A. | CATCTGTCACTGCACT |
| sequence | | | target site-A2 | | | TACCAC |
| of FAS | | | | | | |
| target | | | | | | |
| site | | | | | | |
| DNA | — | 5376 | MG29-1-FAS- | Nucleotide | N.A. | AGTAGACTGTTAGTGC |
| sequence | | | target site-B2 | | | CATGAG |
| of FAS | | | | | | |
| target | | | | | | |
| site | | | | | | |
| DNA | — | 5377 | MG29-1-FAS- | Nucleotide | N.A. | ATTTTACAGGTTCTTA |
| sequence | | | target site-C2 | | | CGTCTG |
| of FAS | | | | | | |
| target | | | | | | |
| site | | | | | | |
| DNA | — | 5378 | MG29-1-FAS- | Nucleotide | N.A. | TTTTACAGGTTCTTAC |
| sequence | | | target site-D2 | | | GTCTGT |
| of FAS | | | | | | |
| target | | | | | | |
| site | | | | | | |
| DNA | — | 5379 | MG29-1-FAS- | Nucleotide | N.A. | ACAGGTTCTTACGTCT |
| sequence | | | target site-E2 | | | GTTGCT |
| of FAS | | | | | | |
| target | | | | | | |
| site | | | | | | |
| DNA | — | 5380 | MG29-1-FAS- | Nucleotide | N.A. | CAGGTTCTTACGTCTG |
| sequence | | | target site-F2 | | | TTGCTA |
| of FAS | | | | | | |
| target | | | | | | |
| site | | | | | | |
| DNA | — | 5381 | MG29-1-FAS- | Nucleotide | N.A. | GTGTAACATACCTGGA |
| sequence | | | target site-G2 | | | GGACAG |
| of FAS | | | | | | |
| target | | | | | | |
| site | | | | | | |
| DNA | — | 5382 | MG29-1-FAS- | Nucleotide | N.A. | TGTAACATACCTGGAG |
| sequence | | | target site-H2 | | | GACAGG |
| of FAS | | | | | | |
| target | | | | | | |
| site | | | | | | |
| DNA | — | 5383 | MG29-1-FAS- | Nucleotide | N.A. | GGACGATAATCTAGCA |
| sequence | | | target site-A3 | | | ACAGAC |
| of FAS | | | | | | |
| target | | | | | | |
| site | | | | | | |
| DNA | — | 5384 | MG29-1-FAS- | Nucleotide | N.A. | GACGATAATCTAGCAA |
| sequence | | | target site-B3 | | | CAGACG |
| of FAS | | | | | | |
| target | | | | | | |
| site | | | | | | |
| DNA | — | 5385 | MG29-1-FAS- | Nucleotide | N.A. | TTCCTTGGGCAGGTGA |
| sequence | | | target site-C3 | | | AAGGAA |
| of FAS | | | | | | |
| target | | | | | | |
| site | | | | | | |
| DNA | — | 5386 | MG29-1-FAS- | Nucleotide | N.A. | TCCTTGGGCAGGTGAA |
| sequence | | | target site-D3 | | | AGGAAA |
| of FAS | | | | | | |
| target | | | | | | |
| site | | | | | | |
| DNA | — | 5387 | MG29-1-FAS- | Nucleotide | N.A. | CCTTGGGCAGGTGAAA |
| sequence | | | target site-E3 | | | GGAAAG |
| of FAS | | | | | | |
| target | | | | | | |
| site | | | | | | |
| DNA | — | 5388 | MG29-1-FAS- | Nucleotide | N.A. | CTTGGGCAGGTGAAAG |
| sequence | | | target site-F3 | | | GAAAGC |
| of FAS | | | | | | |
| target | | | | | | |
| site | | | | | | |
| DNA | — | 5389 | MG29-1-FAS- | Nucleotide | N.A. | TCTTCCAAATGCAGAA |
| sequence | | | target site-G3 | | | GATGTA |
| of FAS | | | | | | |
| target | | | | | | |
| site | | | | | | |
| DNA | — | 5390 | MG29-1-FAS- | Nucleotide | N.A. | CTTCCAAATGCAGAAG |
| sequence | | | target site-H3 | | | ATGTAG |
| of FAS | | | | | | |
| target | | | | | | |
| site | | | | | | |
| DNA | — | 5391 | MG29-1-FAS- | Nucleotide | N.A. | TTCCAAATGCAGAAGA |
| sequence | | | target site-A4 | | | TGTAGA |
| of FAS | | | | | | |
| target | | | | | | |
| site | | | | | | |
| DNA | — | 5392 | MG29-1-FAS- | Nucleotide | N.A. | AAGACTCTTACCATGT |
| sequence | | | target site-B4 | | | CCTTCA |
| of FAS | | | | | | |
| target | | | | | | |
| site | | | | | | |
| DNA | — | 5393 | MG29-1-FAS- | Nucleotide | N.A. | AGACTCTTACCATGTC |
| sequence | | | target site-C4 | | | CTTCAT |
| of FAS | | | | | | |
| target | | | | | | |
| site | | | | | | |
| DNA | — | 5394 | MG29-1-FAS- | Nucleotide | N.A. | GAAGAAAAATGGGCTT |
| sequence | | | target site-D4 | | | TGTCTG |
| of FAS | | | | | | |
| target | | | | | | |
| site | | | | | | |
| DNA | — | 5395 | MG29-1-FAS- | Nucleotide | N.A. | TCTGTGTACTCCTTCC |
| sequence | | | target site-E4 | | | CTTCTT |
| of FAS | | | | | | |
| target | | | | | | |
| site | | | | | | |
| DNA | — | 5396 | MG29-1-FAS- | Nucleotide | N.A. | CAAACTGATTTTCTAG |
| sequence | | | target site-F4 | | | GCTTAG |
| of FAS | | | | | | |
| target | | | | | | |
| site | | | | | | |
| DNA | — | 5397 | MG29-1-FAS- | Nucleotide | N.A. | CTAGGCTTAGAAGTGG |
| sequence | | | target site-G4 | | | AAATAA |
| of FAS | | | | | | |
| target | | | | | | |
| site | | | | | | |
| DNA | — | 5398 | MG29-1-FAS- | Nucleotide | N.A. | TAGGCTTAGAAGTGGA |
| sequence | | | target site-H4 | | | AATAAA |
| of FAS | | | | | | |
| target | | | | | | |
| site | | | | | | |
| DNA | — | 5399 | MG29-1-FAS- | Nucleotide | N.A. | TTTGTAACTCTACTGT |
| sequence | | | target site-A5 | | | ATGTGA |
| of FAS | | | | | | |
| target | | | | | | |
| site | | | | | | |
| DNA | — | 5400 | MG29-1-FAS- | Nucleotide | N.A. | TTGTAACTCTACTGTA |
| sequence | | | target site-B5 | | | TGTGAA |
| of FAS | | | | | | |
| target | | | | | | |
| site | | | | | | |
| DNA | — | 5401 | MG29-1-FAS- | Nucleotide | N.A. | TGTAACTCTACTGTAT |
| sequence | | | target site-C5 | | | GTGAAC |
| of FAS | | | | | | |
| target | | | | | | |
| site | | | | | | |
| DNA | — | 5402 | MG29-1-FAS- | Nucleotide | N.A. | GTAACTCTACTGTATG |
| sequence | | | target site-D5 | | | TGAACA |
| of FAS | | | | | | |
| target | | | | | | |
| site | | | | | | |
| DNA | — | 5403 | MG29-1-FAS- | Nucleotide | N.A. | TAACTCTACTGTATGT |
| sequence | | | target site-E5 | | | GAACAC |
| of FAS | | | | | | |
| target | | | | | | |
| site | | | | | | |
| DNA | — | 5404 | MG29-1-FAS- | Nucleotide | N.A. | GTTTACATCTGCACTT |
| sequence | | | target site-F5 | | | GGTATT |
| of FAS | | | | | | |
| target | | | | | | |
| site | | | | | | |
| DNA | — | 5405 | MG29-1-FAS- | Nucleotide | N.A. | CATCTGCACTTGGTAT |
| sequence | | | target site-G5 | | | TCTGGG |
| of FAS | | | | | | |
| target | | | | | | |
| site | | | | | | |
| DNA | — | 5406 | MG29-1-FAS- | Nucleotide | N.A. | TCCTGTATTTTTTTTTC |
| sequence | | | target site-H5 | | | TAGAT |
| of FAS | | | | | | |
| target | | | | | | |
| site | | | | | | |
| DNA | — | 5407 | MG29-1-FAS- | Nucleotide | N.A. | TTTTTCTAGATGTGAA |
| sequence | | | target site-A6 | | | CATGGA |
| of FAS | | | | | | |
| target | | | | | | |
| site | | | | | | |
| DNA | — | 5408 | MG29-1-FAS- | Nucleotide | N.A. | TTTTCTAGATGTGAAC |
| sequence | | | target site-B6 | | | ATGGAA |
| of FAS | | | | | | |
| target | | | | | | |
| site | | | | | | |
| DNA | — | 5409 | MG29-1-FAS- | Nucleotide | N.A. | TTTCTAGATGTGAACA |
| sequence | | | target site-C6 | | | TGGAAT |
| of FAS | | | | | | |
| target | | | | | | |
| site | | | | | | |
| DNA | — | 5410 | MG29-1-FAS- | Nucleotide | N.A. | TTCTAGATGTGAACAT |
| sequence | | | target site-D6 | | | GGAATC |
| of FAS | | | | | | |
| target | | | | | | |
| site | | | | | | |
| DNA | — | 5411 | MG29-1-FAS- | Nucleotide | N.A. | TCTAGATGTGAACATG |
| sequence | | | target site-E6 | | | GAATCA |
| of FAS | | | | | | |
| target | | | | | | |
| site | | | | | | |
| DNA | — | 5412 | MG29-1-FAS- | Nucleotide | N.A. | CTAGATGTGAACATGG |
| sequence | | | target site-F6 | | | AATCAT |
| of FAS | | | | | | |
| target | | | | | | |
| site | | | | | | |
| DNA | — | 5413 | MG29-1-FAS- | Nucleotide | N.A. | TAGATGTGAACATGGA |
| sequence | | | target site-G6 | | | ATCATC |
| of FAS | | | | | | |
| target | | | | | | |
| site | | | | | | |
| DNA | — | 5414 | MG29-1-FAS- | Nucleotide | N.A. | CACTTGGTGTTGCTGG |
| sequence | | | target site-H6 | | | TGAGTG |
| of FAS | | | | | | |
| target | | | | | | |
| site | | | | | | |
| DNA | — | 5415 | MG29-1-FAS- | Nucleotide | N.A. | TCTTCTTCTTTTGCCA |
| sequence | | | target site-A7 | | | ATTCCA |
| of FAS | | | | | | |
| target | | | | | | |
| site | | | | | | |
| DNA | — | 5416 | MG29-1-FAS- | Nucleotide | N.A. | GCCAATTCCACTAATT |
| sequence | | | target site-B7 | | | GTTTGG |
| of FAS | | | | | | |
| target | | | | | | |
| site | | | | | | |
| DNA | — | 5417 | MG29-1-FAS- | Nucleotide | N.A. | CCAATTCCACTAATTG |
| sequence | | | target site-C7 | | | TTTGGG |
| of FAS | | | | | | |
| target | | | | | | |
| site | | | | | | |
| DNA | — | 5418 | MG29-1-FAS- | Nucleotide | N.A. | AACAAAGCAAGAACTT |
| sequence | | | target site-D7 | | | ACCCCA |
| of FAS | | | | | | |
| target | | | | | | |
| site | | | | | | |
| DNA | — | 5419 | MG29-1-FAS- | Nucleotide | N.A. | TTTGTTCTTTCAGTGA |
| sequence | | | target site-E7 | | | AGAGAA |
| of FAS | | | | | | |
| target | | | | | | |
| site | | | | | | |
| DNA | — | 5420 | MG29-1-FAS- | Nucleotide | N.A. | TTCTTTCAGTGAAGAG |
| sequence | | | target site-F7 | | | AAAGGA |
| of FAS | | | | | | |
| target | | | | | | |
| site | | | | | | |
| DNA | — | 5421 | MG29-1-FAS- | Nucleotide | N.A. | AGTGAAGAGAAAGGA |
| sequence | | | target site-G7 | | | AGTACAG |
| of FAS | | | | | | |
| target | | | | | | |
| site | | | | | | |
| DNA | — | 5422 | MG29-1-FAS- | Nucleotide | N.A. | AATACCTACAGGATTT |
| sequence | | | target site-H7 | | | AAAGTT |
| of FAS | | | | | | |
| target | | | | | | |
| site | | | | | | |
| DNA | — | 5423 | MG29-1-FAS- | Nucleotide | N.A. | AAGTTGGAGATTCATG |
| sequence | | | target site-A8 | | | AGAACC |
| of FAS | | | | | | |
| target | | | | | | |
| site | | | | | | |
| DNA | — | 5424 | MG29-1-FAS- | Nucleotide | N.A. | CCTTTCTGTGCTTTCT |
| sequence | | | target site-B8 | | | GCATGT |
| of FAS | | | | | | |
| target | | | | | | |
| site | | | | | | |
| DNA | — | 5425 | MG29-1-FAS- | Nucleotide | N.A. | CTTTCTGTGCTTTCTG |
| sequence | | | target site-C8 | | | CATGTT |
| of FAS | | | | | | |
| target | | | | | | |
| site | | | | | | |
| DNA | — | 5426 | MG29-1-FAS- | Nucleotide | N.A. | TGTGCTTTCTGCATGT |
| sequence | | | target site-D8 | | | TTTCTG |
| of FAS | | | | | | |
| target | | | | | | |
| site | | | | | | |
| DNA | — | 5427 | MG29-1-FAS- | Nucleotide | N.A. | TGCATGTTTTCTGTAC |
| sequence | | | target site-E8 | | | TTCCTT |
| of FAS | | | | | | |
| target | | | | | | |
| site | | | | | | |
| DNA | — | 5428 | MG29-1-FAS- | Nucleotide | N.A. | CTGTACTTCCTTTCTC |
| sequence | | | target site-F8 | | | TTCACT |
| of FAS | | | | | | |
| target | | | | | | |
| site | | | | | | |
| DNA | — | 5429 | MG29-1-FAS- | Nucleotide | N.A. | TTGCTTTCTAGGAAAC |
| sequence | | | target site-G8 | | | AGTGGC |
| of FAS | | | | | | |
| target | | | | | | |
| site | | | | | | |
| DNA | — | 5430 | MG29-1-FAS- | Nucleotide | N.A. | TGCTTTCTAGGAAACA |
| sequence | | | target site-H8 | | | GTGGCA |
| of FAS | | | | | | |
| target | | | | | | |
| site | | | | | | |
| DNA | — | 5431 | MG29-1-FAS- | Nucleotide | N.A. | GCTTTCTAGGAAACAG |
| sequence | | | target site-A9 | | | TGGCAA |
| of FAS | | | | | | |
| target | | | | | | |
| site | | | | | | |
| DNA | — | 5432 | MG29-1-FAS- | Nucleotide | N.A. | CTTTCTAGGAAACAGT |
| sequence | | | target site-B9 | | | GGCAAT |
| of FAS | | | | | | |
| target | | | | | | |
| site | | | | | | |
| DNA | — | 5433 | MG29-1-FAS- | Nucleotide | N.A. | TAGGAAACAGTGGCAA |
| sequence | | | target site-C9 | | | TAAATT |
| of FAS | | | | | | |
| target | | | | | | |
| site | | | | | | |
| DNA | — | 5434 | MG29-1-FAS- | Nucleotide | N.A. | AGACTATTTTCTATTTT |
| sequence | | | target site-D9 | | | TCAGA |
| of FAS | | | | | | |
| target | | | | | | |
| site | | | | | | |
| DNA | — | 5435 | MG29-1-FAS- | Nucleotide | N.A. | CTATTTTTCAGATGTT |
| sequence | | | target site-E9 | | | GACTTG |
| of FAS | | | | | | |
| target | | | | | | |
| site | | | | | | |
| DNA | — | 5436 | MG29-1-FAS- | Nucleotide | N.A. | TATTTTTCAGATGTTG |
| sequence | | | target site-F9 | | | ACTTGA |
| of FAS | | | | | | |
| target | | | | | | |
| site | | | | | | |
| DNA | — | 5437 | MG29-1-FAS- | Nucleotide | N.A. | TCAGATGTTGACTTGA |
| sequence | | | target site-G9 | | | GTAAAT |
| of FAS | | | | | | |
| target | | | | | | |
| site | | | | | | |
| DNA | — | 5438 | MG29-1-FAS- | Nucleotide | N.A. | CAGATGTTGACTTGAG |
| sequence | | | target site-H9 | | | TAAATA |
| of FAS | | | | | | |
| target | | | | | | |
| site | | | | | | |
| DNA | — | 5439 | MG29-1-FAS- | Nucleotide | N.A. | AGATGTTGACTTGAGT |
| sequence | | | target site-A10 | | | AAATAT |
| of FAS | | | | | | |
| target | | | | | | |
| site | | | | | | |
| DNA | — | 5440 | MG29-1-FAS- | Nucleotide | N.A. | TTCGAAAGAATGGTGT |
| sequence | | | target site-B10 | | | CAATGA |
| of FAS | | | | | | |
| target | | | | | | |
| site | | | | | | |
| DNA | — | 5441 | MG29-1-FAS- | Nucleotide | N.A. | TACTCTTGCAGAGAAA |
| sequence | | | target site-C10 | | | ATTCAG |
| of FAS | | | | | | |
| target | | | | | | |
| site | | | | | | |
| DNA | — | 5442 | MG29-1-FAS | Nucleotide | N.A. | TTGTTTTTCACTCTAG |
| sequence | | | target site-D10 | | | ACCAAG |
| of FAS | | | | | | |
| target | | | | | | |
| site | | | | | | |
| DNA | — | 5443 | MG29-1-FAS- | Nucleotide | N.A. | TCACTCTAGACCAAGC |
| sequence | | | target site-E10 | | | TTTGGA |
| of FAS | | | | | | |
| target | | | | | | |
| site | | | | | | |
| DNA | — | 5444 | MG29-1-FAS- | Nucleotide | N.A. | CACTCTAGACCAAGCT |
| sequence | | | target site-F10 | | | TTGGAT |
| of FAS | | | | | | |
| target | | | | | | |
| site | | | | | | |
| DNA | — | 5445 | MG29-1-FAS- | Nucleotide | N.A. | ACTCTAGACCAAGCTT |
| sequence | | | target site-G10 | | | TGGATT |
| of FAS | | | | | | |
| target | | | | | | |
| site | | | | | | |
| DNA | — | 5446 | MG29-1-FAS- | Nucleotide | N.A. | GATTTCATTTCTGAAG |
| sequence | | | target site-H10 | | | TTTGAA |
| of FAS | | | | | | |
| target | | | | | | |
| site | | | | | | |
| DNA | — | 5447 | MG29-1-FAS- | Nucleotide | N.A. | ATTTCTGAAGTTTGAA |
| sequence | | | target site-A11 | | | TTTTCT |
| of FAS | | | | | | |
| target | | | | | | |
| site | | | | | | |
| DNA | — | 5448 | MG29-1-FAS- | Nucleotide | N.A. | TGAAGTTTGAATTTTC |
| sequence | | | target site-B11 | | | TGAGTC |
| of FAS | | | | | | |
| target | | | | | | |
| site | | | | | | |
| DNA | — | 5449 | MG29-1-FAS- | Nucleotide | N.A. | AATTTTCTGAGTCACT |
| sequence | | | target site-C11 | | | AGTAAT |
| of FAS | | | | | | |
| target | | | | | | |
| site | | | | | | |
| DNA | — | 5450 | MG29-1-FAS- | Nucleotide | N.A. | CTGAGTCACTAGTAAT |
| sequence | | | target site-D11 | | | GTCCTT |
| of FAS | | | | | | |
| target | | | | | | |
| site | | | | | | |
| DNA | — | 5451 | MG29-1-FAS- | Nucleotide | N.A. | TGAGTCACTAGTAATG |
| sequence | | | target site-E11 | | | TCCTTG |
| of FAS | | | | | | |
| target | | | | | | |
| site | | | | | | |
| DNA | — | 5452 | MG29-1-FAS- | Nucleotide | N.A. | CTCTGCAAGAGTACAA |
| sequence | | | target site-F11 | | | AGATTG |
| of FAS | | | | | | |
| target | | | | | | |
| site | | | | | | |
| DNA | — | 5453 | MG29-1-FAS- | Nucleotide | N.A. | TCTGCAAGAGTACAAA |
| sequence | | | target site-G11 | | | GATTGG |
| of FAS | | | | | | |
| target | | | | | | |
| site | | | | | | |
| DNA | — | 5454 | MG29-1-FAS- | Nucleotide | N.A. | TTGAGATCTTTAATCA |
| sequence | | | target site-H11 | | | ATGTGT |
| of FAS | | | | | | |
| target | | | | | | |
| site | | | | | | |
| DNA | — | 5455 | MG29-1-FAS- | Nucleotide | N.A. | TGAGATCTTTAATCAA |
| sequence | | | target site-A12 | | | TGTGTC |
| of FAS | | | | | | |
| target | | | | | | |
| site | | | | | | |
| DNA | — | 5456 | MG29-1-FAS- | Nucleotide | N.A. | GAGATCTTTAATCAAT |
| sequence | | | target site-B12 | | | GTGTCA |
| of FAS | | | | | | |
| target | | | | | | |
| site | | | | | | |
| DNA | — | 5457 | MG29-1-FAS- | Nucleotide | N.A. | AGATCTTTAATCAATG |
| sequence | | | target site-C12 | | | TGTCAT |
| of FAS | | | | | | |
| target | | | | | | |
| site | | | | | | |
| DNA | — | 5458 | MG29-1-FAS- | Nucleotide | N.A. | ATCAATGTGTCATACG |
| sequence | | | target site-D12 | | | CTTCTT |
| of FAS | | | | | | |
| target | | | | | | |
| site | | | | | | |
| DNA | — | 5459 | MG29-1-FAS- | Nucleotide | N.A. | TTTCCATGAAGTTGAT |
| sequence | | | target site-E12 | | | GCCAAT |
| of FAS | | | | | | |
| target | | | | | | |
| site | | | | | | |
| DNA | — | 5460 | MG29-1-FAS- | Nucleotide | N.A. | CATGAAGTTGATGCCA |
| sequence | | | target site-F12 | | | ATTACG |
| of FAS | | | | | | |
| target | | | | | | |
| site | | | | | | |
| DNA | — | 5461 | MG29-1-FAS- | Nucleotide | N.A. | TGTTCTGCTGTGTCTT |
| sequence | | | target site-G12 | | | GGACAT |
| of FAS | | | | | | |
| target | | | | | | |
| site | | | | | | |
| DNA | — | 5462 | MG29-1-FAS- | Nucleotide | N.A. | GGCTTCATTGACACCA |
| sequence | | | target site-H12 | | | TTCTTT |
| of FAS | | | | | | |
| target | | | | | | |
| site | | | | | | |
| DNA | — | 5463 | MG29-1-FAS- | Nucleotide | N.A. | GCTTCATTGACACCAT |
| sequence | | | target site-A13 | | | TCTTTC |
| of FAS | | | | | | |
| target | | | | | | |
| site | | | | | | |
| DNA | — | 5464 | MG29-1-FAS- | Nucleotide | N.A. | GAACAAAGCCTTTAAC |
| sequence | | | target site-B13 | | | TTGACT |
| of FAS | | | | | | |
| target | | | | | | |
| site | | | | | | |
| DNA | — | 5465 | MG29-1-FAS- | Nucleotide | N.A. | ACTTGACTTAGTGTCA |
| sequence | | | target site-C13 | | | TGACTC |
| of FAS | | | | | | |
| target | | | | | | |
| site | | | | | | |
| MG29-1 | — | 5466 | MG29-1-PD-1- | Nucleotide | N.A. | /AltR1/rUrArArUrUrUrCr |
| sgRNA | | | sgRNA-A1 | | | UrArCrUrGrUrUrGrUrAr |
| targeting | | | | | | GrArUrCrUrArGrCrGrG |
| PD-1 | | | | | | rArArUrGrGrGrCrArCr |
| | | | | | | CrUrCrArUrC/AltR2/ |
| MG29-1 | — | 5467 | MG29-1-PD-1- | Nucleotide | N.A. | /AltR1/rUrArArUrUrUrCr |
| sgRNA | | | sgRNA-B1 | | | UrArCrUrGrUrUrGrUrAr |
| targeting | | | | | | GrArUrCrArGrUrGrGrC |
| PD-1 | | | | | | rGrArGrArGrArArGrAr |
| | | | | | | CrCrCrCrGrG/AltR2/ |
| MG29-1 | — | 5468 | MG29-1-PD-1- | Nucleotide | N.A. | /AltR1/rUrArArUrUrUrCr |
| sgRNA | | | sgRNA-C1 | | | UrArCrUrGrUrUrGrUrAr |
| targeting | | | | | | GrArUrUrCrCrUrCrArAr |
| PD-1 | | | | | | ArGrArArGrGrArGrGrA |
| | | | | | | rCrCrCrCrU/AltR2/ |
| MG29-1 | — | 5469 | MG29-1-PD-1- | Nucleotide | N.A. | /AltR1/rUrArArUrUrUrCr |
| sgRNA | | | sgRNA-D1 | | | UrArCrUrGrUrUrGrUrAr |
| targeting | | | | | | GrArUrUrCrUrGrCrArG |
| PD-1 | | | | | | rGrGrArCrArArUrArGr |
| | | | | | | GrArGrCrCrA/AltR2/ |
| MG29-1 | — | 5470 | MG29-1-PD-1- | Nucleotide | N.A. | /AltR1/rUrArArUrUrUrCr |
| sgRNA | | | sgRNA-E1 | | | UrArCrUrGrUrUrGrUrAr |
| targeting | | | | | | GrArUrGrArArCrUrGrG |
| PD-1 | | | | | | rCrCrGrGrCrUrGrGrCr |
| | | | | | | CrUrGrGrGrU/AltR2/ |
| MG29-1 | — | 5471 | MG29-1-PD-1- | Nucleotide | N.A. | /AltR1/rUrArArUrUrUrCr |
| sgRNA | | | sgRNA-F1 | | | UrArCrUrGrUrUrGrUrAr |
| targeting | | | | | | GrArUrUrGrCrCrCrUrUr |
| PD-1 | | | | | | CrCrArGrArGrArGrArA |
| | | | | | | rGrGrGrCrA/AltR2/ |
| MG29-1 | — | 5472 | MG29-1-PD-1- | Nucleotide | N.A. | /AltR1/rUrArArUrUrUrCr |
| sgRNA | | | sgRNA-G1 | | | UrArCrUrGrUrUrGrUrAr |
| targeting | | | | | | GrArUrArUrCrUrGrCrG |
| PD-1 | | | | | | rCrCrUrUrGrGrGrGrGr |
| | | | | | | CrCrArGrGrG/AltR2/ |
| MG29-1 | — | 5473 | MG29-1-PD-1- | Nucleotide | N.A. | /AltR1/rUrArArUrUrUrCr |
| sgRNA | | | sgRNA-H1 | | | UrArCrUrGrUrUrGrUrAr |
| targeting | | | | | | GrArUrGrCrArCrGrArA |
| PD-1 | | | | | | rGrCrUrCrUrCrCrGrArU |
| | | | | | | rGrUrGrUrU/AltR2/ |
| DNA | — | 5474 | MG29-1-PD-1- | Nucleotide | N.A. | ATCTGCGCCTTGGGGG |
| sequence | | | target site-A1 | | | CCAGGG |
| of PD-1 | | | | | | |
| target | | | | | | |
| site | | | | | | |
| DNA | — | 5475 | MG29-1-PD-1- | Nucleotide | N.A. | GCACGAAGCTCTCCGA |
| sequence | | | target site-B1 | | | TGTGTT |
| of PD-1 | | | | | | |
| target | | | | | | |
| site | | | | | | |
| DNA | — | 5476 | MG29-1-PD-1- | Nucleotide | N.A. | GAACTGGCCGGCTGG |
| sequence | | | target site-C1 | | | CCTGGGT |
| of PD-1 | | | | | | |
| target | | | | | | |
| site | | | | | | |
| DNA | — | 5477 | MG29-1-PD-1- | Nucleotide | N.A. | TGCCCTTCCAGAGAGA |
| sequence | | | target site-D1 | | | AGGGCA |
| of PD-1 | | | | | | |
| target | | | | | | |
| site | | | | | | |
| DNA | — | 5478 | MG29-1-PD-1- | Nucleotide | N.A. | TCTGCAGGGACAATAG |
| sequence | | | target site-E1 | | | GAGCCA |
| of PD-1 | | | | | | |
| target | | | | | | |
| site | | | | | | |
| DNA | — | 5479 | MG29-1-PD-1- | Nucleotide | N.A. | TCCTCAAAGAAGGAGG |
| sequence | | | target site-F1 | | | ACCCCT |
| of PD-1 | | | | | | |
| target | | | | | | |
| site | | | | | | |
| DNA | — | 5480 | MG29-1-PD-1- | Nucleotide | N.A. | CAGTGGCGAGAGAAG |
| sequence | | | target site-G1 | | | ACCCCGG |
| of PD-1 | | | | | | |
| target | | | | | | |
| site | | | | | | |
| DNA | — | 5481 | MG29-1-PD-1- | Nucleotide | N.A. | CTAGCGGAATGGGCAC |
| sequence | | | target site-H1 | | | CTCATC |
| of PD-1 | | | | | | |
| target | | | | | | |
| site | | | | | | |
| MG29-1 | — | 5680 | MG29-1 mRNA | Nucleotide | N.A. | GAAAAGCCAGCTCCAG |
| containing | | | | | | CAGGCGCTGCTCACTC |
| 5′ | | | | | | CTCCCCATCCTCTCCC |
| UTR, | | | | | | TCTGTCCCTCTGTCCC |
| NLS, | | | | | | TCTGACCCTGCACTGT |
| CDS, | | | | | | CCCAGCACCATGGCCC |
| NLS, | | | | | | CTAAGAAGAAGAGAAA |
| 3′UTR, | | | | | | AGTCGGCGGAGGCGG |
| polyA | | | | | | CAGCTTCAACAACTTC |
| tail | | | | | | ATCAAGAAGTACAGCC |
| | | | | | | TGCAGAAAACCCTGCG |
| | | | | | | CTTCGAGCTGAAGCCT |
| | | | | | | GTGGGCGAGACAGCC |
| | | | | | | GACTACATCGAGGACT |
| | | | | | | TCAAGAGCGAGTACCT |
| | | | | | | GAAGGACACCGTGCTG |
| | | | | | | AAGGACGAGCAGAGA |
| | | | | | | GCCAAGGACTACCAAG |
| | | | | | | AGATCAAGACCCTGAT |
| | | | | | | CGACGATTACCACCGC |
| | | | | | | GAGTACATCGAAGAGT |
| | | | | | | GCCTGAGAGAACCCGT |
| | | | | | | GGACAAGAAAACCGG |
| | | | | | | CGAGATCCTGGACTTC |
| | | | | | | ACCCAGGACCTGGAAG |
| | | | | | | ATGCCTTCAGCTACTA |
| | | | | | | CCAGAAGCTGAAAGAG |
| | | | | | | AACCCCACCGAGAACA |
| | | | | | | GAGTCGGCTGGGAGA |
| | | | | | | AAGAGCAAGAGAGCCT |
| | | | | | | GAGGAAGAAGCTGGT |
| | | | | | | CACCTCCTTCGTGGGC |
| | | | | | | AACGACGGCCTGTTCA |
| | | | | | | AGAAAGAGTTCATCAC |
| | | | | | | CAGGGACCTGCCTGAG |
| | | | | | | TGGCTGCAGAAGAAAG |
| | | | | | | GACTCTGGGGCGAGTA |
| | | | | | | CAAGGACACAGTGGAA |
| | | | | | | AACTTCAAGAAGTTCA |
| | | | | | | CCACCTACTTCAGCGG |
| | | | | | | CTTCCACGAGAACCGG |
| | | | | | | AAGAACATGTACACCG |
| | | | | | | CCGAGGCTCAGAGCAC |
| | | | | | | CGCTATCGCCAACAGA |
| | | | | | | CTGATGAACGACAACC |
| | | | | | | TGCCTAAGTTCTTTAA |
| | | | | | | CAACTACCTGGCCTAC |
| | | | | | | CAGACCATCAAAGAGA |
| | | | | | | AGCACCCCGACCTGGT |
| | | | | | | GTTCAGACTGGATGAT |
| | | | | | | GCTCTGCTGCAGGCCG |
| | | | | | | CTGGCGTGGAACATCT |
| | | | | | | GGATGAGGCTTTCCAG |
| | | | | | | CCTAGATACTTCAGCA |
| | | | | | | GACTGTTCGCCCAGAG |
| | | | | | | CGGCATCACCGCTTTC |
| | | | | | | AACGAGCTGATCGGCG |
| | | | | | | GCAGAACCACAGAGAA |
| | | | | | | CGGCGAGAAGATCCA |
| | | | | | | GGGCCTGAACGAGCA |
| | | | | | | GATCAACCTGTACAGA |
| | | | | | | CAGCAGAACCCCGAGA |
| | | | | | | AGGCCAAGGGCTTCCC |
| | | | | | | CAGATTCATGCCTCTG |
| | | | | | | TTCAAGCAGATCCTGA |
| | | | | | | GCGACAGAGAGACAC |
| | | | | | | ACAGCTTTCTGCCCGA |
| | | | | | | CGCCTTCGAGAACGAC |
| | | | | | | AAAGAGCTGCTCCAGG |
| | | | | | | CTCTGAGAGACTACGT |
| | | | | | | GGACGCCGCCACATCT |
| | | | | | | GAGGAAGGCATGATCA |
| | | | | | | GCCAGCTGAACAAGGC |
| | | | | | | CATGAACCAGTTCGTG |
| | | | | | | ACCGCCGACCTGAAGA |
| | | | | | | GAGTGTACATCAAGAG |
| | | | | | | CGCCGCTCTGACCAGC |
| | | | | | | CTGAGCCAAGAGCTGT |
| | | | | | | TCCACTTCTTCGGCGT |
| | | | | | | GATCAGCGACGCTATC |
| | | | | | | GCTTGGTACGCCGAGA |
| | | | | | | AGAGACTGAGCCCCAA |
| | | | | | | GAAGGCCCAAGAGTCT |
| | | | | | | TTCCTGAAGCAAGAGG |
| | | | | | | TGTACGCCATCGAGGA |
| | | | | | | ACTGAACCAGGCTGTC |
| | | | | | | GTGGGCTACATCGACC |
| | | | | | | AGCTGGAAGATCAGAG |
| | | | | | | CGAGCTGCAGCAACTG |
| | | | | | | CTGGTGGACCTGCCAG |
| | | | | | | ATCCTCAGAAACCCGT |
| | | | | | | GTCCAGCTTCATCCTG |
| | | | | | | ACACACTGGCAGAAGT |
| | | | | | | CTCAAGAGCCCCTGCA |
| | | | | | | GGCAGTGATCGCCAAG |
| | | | | | | GTGGAACCTCTGTTCG |
| | | | | | | AACTGGAAGAACTGAG |
| | | | | | | CAAGAACAAGAGGGC |
| | | | | | | CCCAAAGCACGACAAG |
| | | | | | | GACCAAGGCGGCGAG |
| | | | | | | GGATTTCAGCAGGTCG |
| | | | | | | ACGCCATCAAGAACAT |
| | | | | | | GCTGGACGCCTTCATG |
| | | | | | | GAAGTGTCCCACGCTA |
| | | | | | | TCAAGCCCCTGTACCT |
| | | | | | | GGTCAAGGGAAGAAA |
| | | | | | | GGCCATCGACATGCCC |
| | | | | | | GACGTGGACACCGGCT |
| | | | | | | TCTACGCTGATTTCGC |
| | | | | | | CGAGGCCTACAGCGCC |
| | | | | | | TACGAGCAAGTGACAG |
| | | | | | | TGTCCCTGTACAACAA |
| | | | | | | GACCAGAAACCACCTG |
| | | | | | | TCCAAGAAGCCCTTCA |
| | | | | | | GCAAGGACAAGATCAA |
| | | | | | | GATCAACTTCGACGCC |
| | | | | | | CCTACACTGCTGAACG |
| | | | | | | GCTGGGACCTGAACAA |
| | | | | | | AGAGAGCGACAACAA |
| | | | | | | GTCCATCATCCTGCGG |
| | | | | | | AAGGACGGCAACTTCT |
| | | | | | | ACCTGGCAATCATGCA |
| | | | | | | CCCCAAGCACACCAAG |
| | | | | | | GTGTTCGACTGCTACT |
| | | | | | | CTGCCTCTGAGGCTGC |
| | | | | | | CGGCAAGTGCTACGAG |
| | | | | | | AAGATGAACTACAAGC |
| | | | | | | TGCTGAGCGGCGCCAA |
| | | | | | | CAAGATGCTGCCTAAG |
| | | | | | | GTGTTCTTTAGCAAGA |
| | | | | | | AGGGCATCGAGACATT |
| | | | | | | CAGCCCTCCACAAGAA |
| | | | | | | ATCCTGGACCTGTACA |
| | | | | | | AGAACAACGAGCATAA |
| | | | | | | GAAGGGCGCCACCTTC |
| | | | | | | AAGCTGGAATCCTGCC |
| | | | | | | ACAAGCTGATCGATTT |
| | | | | | | CTTCAAGCGGAACATC |
| | | | | | | CCCAAGTACAAGGTGC |
| | | | | | | ACCCTACCGACAACTT |
| | | | | | | TGGCTGGGACGTGTTC |
| | | | | | | GGCTTTCACTTCAGCC |
| | | | | | | CTACCAGCAGCTACGG |
| | | | | | | CGACCTGTCTGGCTTC |
| | | | | | | TACAGAGAGGTGGAA |
| | | | | | | GCCCAGGGATACAAGC |
| | | | | | | TGTGGTTCAGCGACGT |
| | | | | | | GTCCGAGGCTTACATC |
| | | | | | | AACAAATGCGTGGAAG |
| | | | | | | AGGGCAAGCTGTTCCT |
| | | | | | | GTTCCAAATCTACAAC |
| | | | | | | AAGGACTTCTCCCCTA |
| | | | | | | ACTCCACCGGCAAGCC |
| | | | | | | CAACCTGCACACCCTG |
| | | | | | | TATTGGAAGGGCCTGT |
| | | | | | | TCGAGCCCGAGAACCT |
| | | | | | | GAAAGACGTGGTGCTG |
| | | | | | | AAGCTGAATGGCGAG |
| | | | | | | GCCGAGATCTTCTACC |
| | | | | | | GGAAGCACAGCATCAA |
| | | | | | | GCACGAGGACAAGAC |
| | | | | | | CATCCACAGAGCTAAG |
| | | | | | | GACCCTATCGCTAACA |
| | | | | | | AGAACGCTGACAACCC |
| | | | | | | CAAGAAACAGAGCGTG |
| | | | | | | TTCGATTACGACATCA |
| | | | | | | TCAAGGATAAGCGGTA |
| | | | | | | TACCCAGGACAAGTTC |
| | | | | | | TTCTTCCACGTGCCAA |
| | | | | | | TCAGCCTGAACTTCAA |
| | | | | | | AAGCCAGGGCGTCGT |
| | | | | | | GCGGTTCAACGATAAG |
| | | | | | | ATCAACGGCCTGCTGG |
| | | | | | | CCGCTCAGGACGATGT |
| | | | | | | GCATGTGATCGGCATC |
| | | | | | | GACAGAGGCGAGAGA |
| | | | | | | CATCTGCTGTACTACA |
| | | | | | | CCGTGGTCAACGGCAA |
| | | | | | | GGGCGAAGTGGTGGA |
| | | | | | | ACAGGGCAGCCTGAAT |
| | | | | | | CAGGTGGCCACAGATC |
| | | | | | | AGGGCTACGTGGTGG |
| | | | | | | ATTACCAGCAGAAGCT |
| | | | | | | GCACGCCAAAGAGAAA |
| | | | | | | GAACGCGACCAGGCC |
| | | | | | | AGAAAGAACTGGTCCA |
| | | | | | | CCATCGAGAACATCAA |
| | | | | | | AGAACTGAAGGCCGG |
| | | | | | | CTACCTGAGCCAGGTG |
| | | | | | | GTGCATAAGCTGGCTC |
| | | | | | | AGCTGATCGTGAAGCA |
| | | | | | | CAACGCCATCGTGTGC |
| | | | | | | CTCGAGGACCTGAATT |
| | | | | | | TCGGCTTCAAGAGGGG |
| | | | | | | CAGATTCAAGGTCGAG |
| | | | | | | AAACAGGTGTACCAGA |
| | | | | | | AGTTCGAGAAGGCTCT |
| | | | | | | GATCGACAAGCTGAAC |
| | | | | | | TACCTCGTGTTCAAAG |
| | | | | | | AGAGAGGCGCCACAC |
| | | | | | | AGGCTGGCGGATACCT |
| | | | | | | GAATGCTTACCAGCTG |
| | | | | | | GCCGCACCTTTCGAGA |
| | | | | | | GCTTTGAGAAGCTGGG |
| | | | | | | CAAGCAGACCGGCATC |
| | | | | | | CTGTACTACGTGCGGA |
| | | | | | | GCGACTACACCAGCAA |
| | | | | | | GATCGACCCTGCTACC |
| | | | | | | GGCTTCGTGGACTTTC |
| | | | | | | TGAAGCCTAAGTACGA |
| | | | | | | GAGCATGGCCAAGAG |
| | | | | | | CAAAGTGTTCTTCGAG |
| | | | | | | TCCTTCGAGCGCATCC |
| | | | | | | AGTGGAACCAGGCCAA |
| | | | | | | AGGCTACTTCGAGTTC |
| | | | | | | GAGTTTGACTACAAGA |
| | | | | | | AGATGTGCCCCAGCAG |
| | | | | | | AAAGTTCGGCGACTAC |
| | | | | | | AGAACCAGATGGGTCG |
| | | | | | | TGTGCACCTTCGGCGA |
| | | | | | | CACCCGCTACCAGAAC |
| | | | | | | AGAAGAAACAAGAGCA |
| | | | | | | GCGGCCAGTGGGAGA |
| | | | | | | CAGAGACAATCGATGT |
| | | | | | | GACAGCCCAGCTGAAA |
| | | | | | | GCCCTGTTCGCCGCTT |
| | | | | | | ACGGCATCACATACAA |
| | | | | | | TCAAGAGGATAACATC |
| | | | | | | AAGGACGCCATTGCCG |
| | | | | | | CCGTGAAGTACACCAA |
| | | | | | | GTTCTACAAGCAGCTG |
| | | | | | | TACTGGCTGCTGAGAC |
| | | | | | | TGACCCTGAGCCTGAG |
| | | | | | | ACACAGCGTGACAGGC |
| | | | | | | ACCGACGAGGATTTCA |
| | | | | | | TCCTGTCTCCAGTGGC |
| | | | | | | CGACGAGAATGGCGT |
| | | | | | | GTTCTTTGACTCTAGG |
| | | | | | | AAGGCCACCGACAAGC |
| | | | | | | AGCCTAAGGACGCTGA |
| | | | | | | TGCTAACGGCGCCTAC |
| | | | | | | CATATCGCCCTGAAAG |
| | | | | | | GCCTGTGGAATCTCCA |
| | | | | | | GCAGATCAGACAGCAC |
| | | | | | | GACTGGAACGTGGAAA |
| | | | | | | AGCCCAAAAAGCTGAA |
| | | | | | | CCTCGCCATGAAGAAC |
| | | | | | | GAAGAGTGGTTCGGCT |
| | | | | | | TCGCTCAGAAGAAGAA |
| | | | | | | GTTTAGAGCCAGCGGC |
| | | | | | | GGCAAGAGGCCTGCC |
| | | | | | | GCTACAAAAAAAGCCG |
| | | | | | | GCCAGGCCAAGAAAAA |
| | | | | | | GAAGTGACCACACCCC |
| | | | | | | CATTCCCCCACTCCAG |
| | | | | | | ATAGAACTTCAGTTAT |
| | | | | | | ATCTCACGTGTCTGGA |
| | | | | | | GTTGGATCCCTTGAAG |
| | | | | | | ACTAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAA |
| MG29-1 | — | 5681 | MG29-1-TRAC- | Nucleotide | N.A. | mU*rArArUrUrUrCrUrA |
| sgRNA | | | sgRNA-35 | | | rCrUrGrUrUrGrUrArGr |
| targeting | | | | | | ArUrGrArGrUrCrUrCrUr |
| TRAC | | | | | | CrArGrCrUrGrGrUrArC |
| | | | | | | rArCrG*mG |
| DNA | — | 5682 | MG29-1-TRAC- | Nucleotide | N.A. | GAGTCTCTCAGCTGGT |
| sequence | | | target site-35 | | | ACACGG |
| of TRAC | | | | | | |
| target | | | | | | |
| site | | | | | | |
| MG29-1 | — | 5683 | MG29-1-TRAC- | Nucleotide | N.A. | /AltR1/rUrArArUrUrUrCr |
| sgRNA | | | sgRNA-35-AltR | | | UrArCrUrGrUrUrGrUrAr |
| targeting | | | | | | GrArUrGrArGrUrCrUrC |
| TRAC | | | | | | rUrCrArGrCrUrGrGrUr |
| | | | | | | ArCrArCrGrG/AltR2/ |
| DNA | — | 5684 | MG29-1-TRAC- | Nucleotide | N.A. | GAGTCTCTCAGCTGGT |
| sequence | | | target site- | | | ACACGG |
| of TRAC | | | 35-AltR | | | |
| target | | | | | | |
| site | | | | | | |
| MG29-1 | — | 5685 | MG29-1-CD38- | Nucleotide | N.A. | mC*mU*mU*rU*rUrArAr |
| sgRNA | | | sgRNA | | | UrUmUmCmUmArCrU*r |
| targeting | | | | | | G*rU*rU*rGrUrArGrArU |
| CD38 | | | | | | rCrCrGrArGrArC/i2FC//i |
| | | | | | | 2FG//i2FU//i2FC//i2FC//12 |
| | | | | | | FU//12FG//12FG//i2FC//i2F |
| | | | | | | G/*/12FC/*/12FG//i2FA/*/12 |
| | | | | | | FU/*mG |
| DNA | — | 5686 | MG29-1-CD38- | Nucleotide | N.A. | CCGAGACCGTCCTGGC |
| sequence | | | target site | | | GCGATG |
| of CD38 | | | | | | |
| target | | | | | | |
| site | | | | | | |
| MG29-1 | — | 5687 | MG29-1-mRNA | Nucleotide | N.A. | ATGGCCCCTAAGAAGA |
| coding | | | coding sequence | | | AGAGAAAAGTCGGCG |
| sequence | | | | | | GAGGCGGCAGCTTCAA |
| used for | | | | | | CAACTTCATCAAGAAG |
| generati | | | | | | TACAGCCTGCAGAAAA |
| on of | | | | | | CCCTGCGCTTCGAGCT |
| mRNA | | | | | | GAAGCCTGTGGGCGA |
| | | | | | | GACAGCCGACTACATC |
| | | | | | | GAGGACTTCAAGAGCG |
| | | | | | | AGTACCTGAAGGACAC |
| | | | | | | CGTGCTGAAGGACGA |
| | | | | | | GCAGAGAGCCAAGGA |
| | | | | | | CTACCAAGAGATCAAG |
| | | | | | | ACCCTGATCGACGATT |
| | | | | | | ACCACCGCGAGTACAT |
| | | | | | | CGAAGAGTGCCTGAGA |
| | | | | | | GAACCCGTGGACAAGA |
| | | | | | | AAACCGGCGAGATCCT |
| | | | | | | GGACTTCACCCAGGAC |
| | | | | | | CTGGAAGATGCCTTCA |
| | | | | | | GCTACTACCAGAAGCT |
| | | | | | | GAAAGAGAACCCCACC |
| | | | | | | GAGAACAGAGTCGGCT |
| | | | | | | GGGAGAAAGAGCAAG |
| | | | | | | AGAGCCTGAGGAAGA |
| | | | | | | AGCTGGTCACCTCCTT |
| | | | | | | CGTGGGCAACGACGG |
| | | | | | | CCTGTTCAAGAAAGAG |
| | | | | | | TTCATCACCAGGGACC |
| | | | | | | TGCCTGAGTGGCTGCA |
| | | | | | | GAAGAAAGGACTCTGG |
| | | | | | | GGCGAGTACAAGGAC |
| | | | | | | ACAGTGGAAAACTTCA |
| | | | | | | AGAAGTTCACCACCTA |
| | | | | | | CTTCAGCGGCTTCCAC |
| | | | | | | GAGAACCGGAAGAAC |
| | | | | | | ATGTACACCGCCGAGG |
| | | | | | | CTCAGAGCACCGCTAT |
| | | | | | | CGCCAACAGACTGATG |
| | | | | | | AACGACAACCTGCCTA |
| | | | | | | AGTTCTTTAACAACTA |
| | | | | | | CCTGGCCTACCAGACC |
| | | | | | | ATCAAAGAGAAGCACC |
| | | | | | | CCGACCTGGTGTTCAG |
| | | | | | | ACTGGATGATGCTCTG |
| | | | | | | CTGCAGGCCGCTGGC |
| | | | | | | GTGGAACATCTGGATG |
| | | | | | | AGGCTTTCCAGCCTAG |
| | | | | | | ATACTTCAGCAGACTG |
| | | | | | | TTCGCCCAGAGCGGCA |
| | | | | | | TCACCGCTTTCAACGA |
| | | | | | | GCTGATCGGCGGCAG |
| | | | | | | AACCACAGAGAACGGC |
| | | | | | | GAGAAGATCCAGGGC |
| | | | | | | CTGAACGAGCAGATCA |
| | | | | | | ACCTGTACAGACAGCA |
| | | | | | | GAACCCCGAGAAGGC |
| | | | | | | CAAGGGCTTCCCCAGA |
| | | | | | | TTCATGCCTCTGTTCA |
| | | | | | | AGCAGATCCTGAGCGA |
| | | | | | | CAGAGAGACACACAGC |
| | | | | | | TTTCTGCCCGACGCCT |
| | | | | | | TCGAGAACGACAAAGA |
| | | | | | | GCTGCTCCAGGCTCTG |
| | | | | | | AGAGACTACGTGGACG |
| | | | | | | CCGCCACATCTGAGGA |
| | | | | | | AGGCATGATCAGCCAG |
| | | | | | | CTGAACAAGGCCATGA |
| | | | | | | ACCAGTTCGTGACCGC |
| | | | | | | CGACCTGAAGAGAGTG |
| | | | | | | TACATCAAGAGCGCCG |
| | | | | | | CTCTGACCAGCCTGAG |
| | | | | | | CCAAGAGCTGTTCCAC |
| | | | | | | TTCTTCGGCGTGATCA |
| | | | | | | GCGACGCTATCGCTTG |
| | | | | | | GTACGCCGAGAAGAG |
| | | | | | | ACTGAGCCCCAAGAAG |
| | | | | | | GCCCAAGAGTCTTTCC |
| | | | | | | TGAAGCAAGAGGTGTA |
| | | | | | | CGCCATCGAGGAACTG |
| | | | | | | AACCAGGCTGTCGTGG |
| | | | | | | GCTACATCGACCAGCT |
| | | | | | | GGAAGATCAGAGCGA |
| | | | | | | GCTGCAGCAACTGCTG |
| | | | | | | GTGGACCTGCCAGATC |
| | | | | | | CTCAGAAACCCGTGTC |
| | | | | | | CAGCTTCATCCTGACA |
| | | | | | | CACTGGCAGAAGTCTC |
| | | | | | | AAGAGCCCCTGCAGGC |
| | | | | | | AGTGATCGCCAAGGTG |
| | | | | | | GAACCTCTGTTCGAAC |
| | | | | | | TGGAAGAACTGAGCAA |
| | | | | | | GAACAAGAGGGCCCC |
| | | | | | | AAAGCACGACAAGGAC |
| | | | | | | CAAGGCGGCGAGGGA |
| | | | | | | TTTCAGCAGGTCGACG |
| | | | | | | CCATCAAGAACATGCT |
| | | | | | | GGACGCCTTCATGGAA |
| | | | | | | GTGTCCCACGCTATCA |
| | | | | | | AGCCCCTGTACCTGGT |
| | | | | | | CAAGGGAAGAAAGGC |
| | | | | | | CATCGACATGCCCGAC |
| | | | | | | GTGGACACCGGCTTCT |
| | | | | | | ACGCTGATTTCGCCGA |
| | | | | | | GGCCTACAGCGCCTAC |
| | | | | | | GAGCAAGTGACAGTGT |
| | | | | | | CCCTGTACAACAAGAC |
| | | | | | | CAGAAACCACCTGTCC |
| | | | | | | AAGAAGCCCTTCAGCA |
| | | | | | | AGGACAAGATCAAGAT |
| | | | | | | CAACTTCGACGCCCCT |
| | | | | | | ACACTGCTGAACGGCT |
| | | | | | | GGGACCTGAACAAAGA |
| | | | | | | GAGCGACAACAAGTCC |
| | | | | | | ATCATCCTGCGGAAGG |
| | | | | | | ACGGCAACTTCTACCT |
| | | | | | | GGCAATCATGCACCCC |
| | | | | | | AAGCACACCAAGGTGT |
| | | | | | | TCGACTGCTACTCTGC |
| | | | | | | CTCTGAGGCTGCCGGC |
| | | | | | | AAGTGCTACGAGAAGA |
| | | | | | | TGAACTACAAGCTGCT |
| | | | | | | GAGCGGCGCCAACAA |
| | | | | | | GATGCTGCCTAAGGTG |
| | | | | | | TTCTTTAGCAAGAAGG |
| | | | | | | GCATCGAGACATTCAG |
| | | | | | | CCCTCCACAAGAAATC |
| | | | | | | CTGGACCTGTACAAGA |
| | | | | | | ACAACGAGCATAAGAA |
| | | | | | | GGGCGCCACCTTCAAG |
| | | | | | | CTGGAATCCTGCCACA |
| | | | | | | AGCTGATCGATTTCTT |
| | | | | | | CAAGCGGAACATCCCC |
| | | | | | | AAGTACAAGGTGCACC |
| | | | | | | CTACCGACAACTTTGG |
| | | | | | | CTGGGACGTGTTCGGC |
| | | | | | | TTTCACTTCAGCCCTA |
| | | | | | | CCAGCAGCTACGGCGA |
| | | | | | | CCTGTCTGGCTTCTAC |
| | | | | | | AGAGAGGTGGAAGCC |
| | | | | | | CAGGGATACAAGCTGT |
| | | | | | | GGTTCAGCGACGTGTC |
| | | | | | | CGAGGCTTACATCAAC |
| | | | | | | AAATGCGTGGAAGAG |
| | | | | | | GGCAAGCTGTTCCTGT |
| | | | | | | TCCAAATCTACAACAA |
| | | | | | | GGACTTCTCCCCTAAC |
| | | | | | | TCCACCGGCAAGCCCA |
| | | | | | | ACCTGCACACCCTGTA |
| | | | | | | TTGGAAGGGCCTGTTC |
| | | | | | | GAGCCCGAGAACCTGA |
| | | | | | | AAGACGTGGTGCTGAA |
| | | | | | | GCTGAATGGCGAGGC |
| | | | | | | CGAGATCTTCTACCGG |
| | | | | | | AAGCACAGCATCAAGC |
| | | | | | | ACGAGGACAAGACCAT |
| | | | | | | CCACAGAGCTAAGGAC |
| | | | | | | CCTATCGCTAACAAGA |
| | | | | | | ACGCTGACAACCCCAA |
| | | | | | | GAAACAGAGCGTGTTC |
| | | | | | | GATTACGACATCATCA |
| | | | | | | AGGATAAGCGGTATAC |
| | | | | | | CCAGGACAAGTTCTTC |
| | | | | | | TTCCACGTGCCAATCA |
| | | | | | | GCCTGAACTTCAAAAG |
| | | | | | | CCAGGGCGTCGTGCG |
| | | | | | | GTTCAACGATAAGATC |
| | | | | | | AACGGCCTGCTGGCCG |
| | | | | | | CTCAGGACGATGTGCA |
| | | | | | | TGTGATCGGCATCGAC |
| | | | | | | AGAGGCGAGAGACAT |
| | | | | | | CTGCTGTACTACACCG |
| | | | | | | TGGTCAACGGCAAGG |
| | | | | | | GCGAAGTGGTGGAAC |
| | | | | | | AGGGCAGCCTGAATCA |
| | | | | | | GGTGGCCACAGATCAG |
| | | | | | | GGCTACGTGGTGGATT |
| | | | | | | ACCAGCAGAAGCTGCA |
| | | | | | | CGCCAAAGAGAAAGAA |
| | | | | | | CGCGACCAGGCCAGA |
| | | | | | | AAGAACTGGTCCACCA |
| | | | | | | TCGAGAACATCAAAGA |
| | | | | | | ACTGAAGGCCGGCTAC |
| | | | | | | CTGAGCCAGGTGGTGC |
| | | | | | | ATAAGCTGGCTCAGCT |
| | | | | | | GATCGTGAAGCACAAC |
| | | | | | | GCCATCGTGTGCCTCG |
| | | | | | | AGGACCTGAATTTCGG |
| | | | | | | CTTCAAGAGGGGCAGA |
| | | | | | | TTCAAGGTCGAGAAAC |
| | | | | | | AGGTGTACCAGAAGTT |
| | | | | | | CGAGAAGGCTCTGATC |
| | | | | | | GACAAGCTGAACTACC |
| | | | | | | TCGTGTTCAAAGAGAG |
| | | | | | | AGGCGCCACACAGGCT |
| | | | | | | GGCGGATACCTGAATG |
| | | | | | | CTTACCAGCTGGCCGC |
| | | | | | | ACCTTTCGAGAGCTTT |
| | | | | | | GAGAAGCTGGGCAAG |
| | | | | | | CAGACCGGCATCCTGT |
| | | | | | | ACTACGTGCGGAGCGA |
| | | | | | | CTACACCAGCAAGATC |
| | | | | | | GACCCTGCTACCGGCT |
| | | | | | | TCGTGGACTTTCTGAA |
| | | | | | | GCCTAAGTACGAGAGC |
| | | | | | | ATGGCCAAGAGCAAAG |
| | | | | | | TGTTCTTCGAGTCCTT |
| | | | | | | CGAGCGCATCCAGTGG |
| | | | | | | AACCAGGCCAAAGGCT |
| | | | | | | ACTTCGAGTTCGAGTT |
| | | | | | | TGACTACAAGAAGATG |
| | | | | | | TGCCCCAGCAGAAAGT |
| | | | | | | TCGGCGACTACAGAAC |
| | | | | | | CAGATGGGTCGTGTGC |
| | | | | | | ACCTTCGGCGACACCC |
| | | | | | | GCTACCAGAACAGAAG |
| | | | | | | AAACAAGAGCAGCGG |
| | | | | | | CCAGTGGGAGACAGA |
| | | | | | | GACAATCGATGTGACA |
| | | | | | | GCCCAGCTGAAAGCCC |
| | | | | | | TGTTCGCCGCTTACGG |
| | | | | | | CATCACATACAATCAA |
| | | | | | | GAGGATAACATCAAGG |
| | | | | | | ACGCCATTGCCGCCGT |
| | | | | | | GAAGTACACCAAGTTC |
| | | | | | | TACAAGCAGCTGTACT |
| | | | | | | GGCTGCTGAGACTGAC |
| | | | | | | CCTGAGCCTGAGACAC |
| | | | | | | AGCGTGACAGGCACC |
| | | | | | | GACGAGGATTTCATCC |
| | | | | | | TGTCTCCAGTGGCCGA |
| | | | | | | CGAGAATGGCGTGTTC |
| | | | | | | TTTGACTCTAGGAAGG |
| | | | | | | CCACCGACAAGCAGCC |
| | | | | | | TAAGGACGCTGATGCT |
| | | | | | | AACGGCGCCTACCATA |
| | | | | | | TCGCCCTGAAAGGCCT |
| | | | | | | GTGGAATCTCCAGCAG |
| | | | | | | ATCAGACAGCACGACT |
| | | | | | | GGAACGTGGAAAAGC |
| | | | | | | CCAAAAAGCTGAACCT |
| | | | | | | CGCCATGAAGAACGAA |
| | | | | | | GAGTGGTTCGGCTTCG |
| | | | | | | CTCAGAAGAAGAAGTT |
| | | | | | | TAGAGCCAGCGGCGG |
| | | | | | | CAAGAGGCCTGCCGCT |
| | | | | | | ACAAAAAAAGCCGGCC |
| | | | | | | AGGCCAAGAAAAAGAA |
| | | | | | | GTGA |
| MG29-1 | — | 5688 | mAlb29-8-44 | Nucleotide | N.A. | mC*mU*mU*U*U*AAUU |
| sgRNA | | | | | | mUmCmUmACU*G*U*U |
| targeting | | | | | | *GUAGAUCUGUAACfGf |
| mouse | | | | | | AfUfCfGfGfGfA*fAfC*fU |
| albumin | | | | | | *fG*fG*fC*mA |
| MG29-1 | — | 5689 | mAlb29-8-50 | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| albumin | | | | | | *G*U*U*GUAGAUCUGU |
| | | | | | | AACfGfAfUfCfGfGfGfAf |
| | | | | | | AfC*fU*fGfG*fC*mA |
| MG29-1 | — | 5690 | mAlb29-8-50b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| albumin | | | | | | *G*U*U*GUAGAUCUGU |
| | | | | | | AACfGfAfUfCfGfGfGfAf |
| | | | | | | AfC*fU*fG*mG |
| MG29-1 | — | 5691 | mAlb29-8-51b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| albumin | | | | | | *G*U*U*GUAGAUCUGU |
| | | | | | | AACGAUCGGGAAC*U* |
| | | | | | | mG*mG |
| MG29-1 | — | 5692 | mAlb29-8-52b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| albumin | | | | | | *G*U*U*GUAGAUCUGU |
| | | | | | | AACGfAUfCGfGGfA*A*f |
| | | | | | | CU*fG*mG |
| MG29-1 | — | 5693 | mAlb29-8-53b | Nucleotide | N.A. | mG*mU*mU*mG*mA*m |
| sgRNA | | | | | | G*mA*mA*mU*mC*mG* |
| targeting | | | | | | mA*mA*mA*mG*mA*m |
| mouse | | | | | | U*mU*mC*mU*mC*mA* |
| albumin | | | | | | mA*mC*mC*mU*mU*U* |
| | | | | | | UAAUUmUmCmUmACU* |
| | | | | | | G*U*U*GUAGAUCUGU |
| | | | | | | AACfGfAfUfCfGfGfGfAf |
| | | | | | | AfC*fU*fG*mG |
| MG29-1 | — | 5694 | mAlb29-8-54b | Nucleotide | N.A. | mG*mU*mU*mG*mA*m |
| sgRNA | | | | | | G*mA*mA*mU*mC*mG* |
| targeting | | | | | | mA*mA*mA*mG*mA*m |
| mouse | | | | | | U*mU*mC*mU*mC*mA* |
| albumin | | | | | | mA*mC*mC*mU*mU*U* |
| | | | | | | UAAUUmUmCmUmACU* |
| | | | | | | G*U*U*GUAGAUCUGU |
| | | | | | | AACGAUCGGGAAC*U* |
| | | | | | | mG*mG |
| Guide | — | 5695 | Chemistry 44 (22 | Nucleotide | N.A. | mC*mU*mU*U*U*AAUU |
| modifica- | | | nt spacer) | | | mUmCmUmACU*G*U*U |
| tion | | | | | | *GUAGAUNNNNNNNfNf |
| chemistry | | | | | | NfNfNfNfNfNfN*fNfN*fN* |
| | | | | | | fN*fN*fN*mN |
| Guide | — | 5696 | Chemistry 50 (22 | Nucleotide | N.A. | mG*mU*mU*GAGAAUC |
| modifica- | | | nt spacer) | | | *mG*mA*mA*mAGAUU |
| tion | | | | | | CUCAAC*mC*mU*mU*U |
| chemistry | | | | | | *UAAUUmUmCmUmACU |
| | | | | | | *G*U*U*GUAGAUNNNN |
| | | | | | | NNNfNfNfNfNfNfNfNfNfN |
| | | | | | | fN*fN*fNfN*fN*mN |
| Guide | — | 5697 | Chemistry 50 (20 | Nucleotide | N.A. | mG*mU*mU*GAGAAUC |
| modifica- | | | nt spacer) | | | *mG*mA*mA*mAGAUU |
| tion | | | | | | CUCAAC*mC*mU*mU*U |
| chemistry | | | | | | *UAAUUmUmCmUmACU |
| | | | | | | *G*U*U*GUAGAUNNNN |
| | | | | | | NNNfNfNfNfNfNfNfNfNfN |
| | | | | | | fN*fN*fN*mN |
| Guide | — | 5698 | Chemistry 51 (20 | Nucleotide | N.A. | mG*mU*mU*GAGAAUC |
| modifica- | | | nt spacer) | | | *mG*mA*mA*mAGAUU |
| tion | | | | | | CUCAAC*mC*mU*mU*U |
| chemistry | | | | | | *UAAUUmUmCmUmACU |
| | | | | | | *G*U*U*GUAGAUNNNN |
| | | | | | | NNNNNNNNNNNNN*N* |
| | | | | | | mN*mN |
| Guide | — | 5699 | Chemistry 52 (20 | Nucleotide | N.A. | mG*mU*mU*GAGAAUC |
| modifica- | | | nt spacer) | | | *mG*mA*mA*mAGAUU |
| tion | | | | | | CUCAAC*mC*mU*mU*U |
| chemistry | | | | | | *UAAUUmUmCmUmACU |
| | | | | | | *G*U*U*GUAGAUNNNN |
| | | | | | | NNNNfNNfNNfNNfN*N*f |
| | | | | | | NN*fN*mN |
| Guide | — | 5700 | Chemistry 53 (20 | Nucleotide | N.A. | mG*mU*mU*mG*mA*m |
| modifica- | | | nt spacer) | | | G*mA*mA*mU*mC*mG* |
| tion | | | | | | mA*mA*mA*mG*mA*m |
| chemistry | | | | | | U*mU*mC*mU*mC*mA* |
| | | | | | | mA*mC*mC*mU*mU*U* |
| | | | | | | UAAUUmUmCmUmACU* |
| | | | | | | G*U*U*GUAGAUNNNNN |
| | | | | | | NNfNfNfNfNfNfNfNfNfNf |
| Guide | — | 5701 | Chemistry 54 (20 | Nucleotide | N.A. | mG*mU*mU*mG*mA*m |
| modifica- | | | nt spacer) | | | G*mA*mA*mU*mC*mG* |
| tion | | | | | | mA*mA*mA*mG*mA*m |
| chemistry | | | | | | U*mU*mC*mU*mC*mA* |
| | | | | | | mA*mC*mC*mU*mU*U* |
| | | | | | | UAAUUmUmCmUmACU* |
| | | | | | | G*U*U*GUAGAUNNNNN |
| | | | | | | NNNNNNNNNNNN*N*m |
| | | | | | | N*mN |
| MG29-1 | — | 5702 | mAlb29-8-37 | Nucleotide | N.A. | mC*mU*mU*U*UAAUU |
| sgRNA | | | | | | mUmCmUmACU*G*U*U |
| targeting | | | | | | *GUAGAUCUGUAACfGf |
| mouse | | | | | | AfUfCfGfGfGfAfAfC*fU*f |
| albumin | | | | | | GfG*fC*mA |
| MG29-1 | — | 5703 | mAlb29-12-44 | Nucleotide | N.A. | mC*mU*mU*U*U*AAUU |
| sgRNA | | | | | | mUmCmUmACU*G*U*U |
| targeting | | | | | | *GUAGAUAGUGUAGfCf |
| mouse | | | | | | AfGfAfGfAfGfG*fAfA*fC |
| albumin | | | | | | *fC*fA*fU*mU |
| MG29-1 | — | 5704 | mH29-29-50 | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HA01 | | | | | | *G*U*U*GUAGAUCCUU |
| | | | | | | AGGfAfGfAfAfAfAfUfGf |
| | | | | | | CfC*fA*fAfA*fU*mC |
| MG29-1 | — | 5705 | mH29-29-50b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HA01 | | | | | | *G*U*U*GUAGAUCCUU |
| | | | | | | AGGfAfGfAfAfAfAfUfGf |
| | | | | | | CfC*fA*fA*mA |
| MG29-1 | — | 5706 | mH29-29-51b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HA01 | | | | | | *G*U*U*GUAGAUCCUU |
| | | | | | | AGGAGAAAAUGCC*A* |
| | | | | | | mA*mA |
| MG29-1 | — | 5707 | mH29-29-52b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HA01 | | | | | | *G*U*U*GUAGAUCCUU |
| | | | | | | AGGAfGAfAAfAUfG*C*f |
| | | | | | | CA*fA*mA |
| MG29-1 | — | 5708 | mH29-29-53b | Nucleotide | N.A. | mG*mU*mU*mG*mA*m |
| sgRNA | | | | | | G*mA*mA*mU*mC*mG* |
| targeting | | | | | | mA*mA*mA*mG*mA*m |
| mouse | | | | | | U*mU*mC*mU*mC*mA* |
| HA01 | | | | | | mA*mC*mC*mU*mU*U* |
| | | | | | | UAAUUmUmCmUmACU* |
| | | | | | | G*U*U*GUAGAUCCUUA |
| | | | | | | GGfAfGfAfAfAfAfUfGfCf |
| | | | | | | C*fA*fA*mA |
| MG29-1 | — | 5709 | mH29-29-54b | Nucleotide | N.A. | mG*mU*mU*mG*mA*m |
| sgRNA | | | | | | G*mA*mA*mU*mC*mG* |
| targeting | | | | | | mA*mA*mA*mG*mA*m |
| mouse | | | | | | U*mU*mC*mU*mC*mA* |
| HA01 | | | | | | mA*mC*mC*mU*mU*U* |
| | | | | | | UAAUUmUmCmUmACU* |
| | | | | | | G*U*U*GUAGAUCCUUA |
| | | | | | | GGAGAAAAUGCC*A*m |
| | | | | | | A*mA |
| MG29-1 | — | 5710 | mH29-29.1_37 | Nucleotide | N.A. | mC*mU*mU*U*UAAUU |
| sgRNA | | | | | | mUmCmUmACU*G*U*U |
| targeting | | | | | | *GUAGAUCCUUAGGfAf |
| mouse | | | | | | GfAfAfAfAfUfG*fCfC*fA |
| HA01 | | | | | | *fA*mA |
| MG29-1 | — | 5711 | mH29-29.2_37 | Nucleotide | N.A. | mC*mU*mU*U*UAAUU |
| sgRNA | | | | | | mUmCmUmACU*G*U*U |
| targeting | | | | | | *GUAGAUCCUUAGGfAf |
| mouse | | | | | | GfAfAfAfAfUfG*fC*fC*f |
| HA01 | | | | | | A*fA*mA |
| MG29-1 | — | 5712 | mAlb29-g8-37- | Nucleotide | N.A. | mG*mU*mC*UAAGACC |
| CRISPR | | | array | | | UmU*mA*mC*U*U*AAU |
| array | | | | | | UmUmCmUmACU*G*U* |
| targeting | | | | | | U*GUAGAUCUGUAACf |
| mouse | | | | | | GfAfUfCfGfGfGfA*fAfC*f |
| albumin | | | | | | U*fG*fG*fC*mAUCUUC |
| | | | | | | AGUCUAAGACCUMU*m |
| | | | | | | A*mC*U*U*AAUUmUmC |
| | | | | | | mUmACU*G*U*U*GUAG |
| | | | | | | AUCUGUAACfGfAfUfCf |
| | | | | | | GfGfGfA*fAfC*fU*fG*fG |
| | | | | | | *fC*mAUCU*mU*mC*m |
| | | | | | | A |
| MG29-1 | — | 5713 | hA29-87-37B | Nucleotide | N.A. | mC*mU*mU*U*UAAUU |
| sgRNA | | | | | | mUmCmUmACU*G*U*U |
| targeting | | | | | | *GUAGAUCGCACUAfAf |
| human | | | | | | GfGfAfAfAfGfU*fGfC*fA |
| albumin | | | | | | *fA*mA |
| MG29-1 | — | 5714 | hA29-78-37B | Nucleotide | N.A. | mC*mU*mU*U*UAAUU |
| sgRNA | | | | | | mUmCmUmACU*G*U*U |
| targeting | | | | | | *GUAGAUUUUUGCGfCf |
| human | | | | | | AfCfUfAfAfGfG*fAfA*fA |
| albumin | | | | | | *fG*mU |
| MG29-1 | — | 5715 | hA29-74-37B | Nucleotide | N.A. | mC*mU*mU*U*UAAUU |
| sgRNA | | | | | | mUmCmUmACU*G*U*U |
| targeting | | | | | | *GUAGAUAAUAAAGfCf |
| human | | | | | | AfUfAfGfUfGfC*fAfA*fU |
| albumin | | | | | | *fG*mG |
| MG29-1 | — | 5716 | hA29-83-37B | Nucleotide | N.A. | mC*mU*mU*U*UAAUU |
| sgRNA | | | | | | mUmCmUmACU*G*U*U |
| targeting | | | | | | *GUAGAUUGAGAUCfAf |
| human | | | | | | AfCfAfGfCfAfC*fAfG*fG |
| albumin | | | | | | *fU*mU |
| MG29-1 | — | 5717 | hA29-84-37B | Nucleotide | N.A. | mC*mU*mU*U*UAAUU |
| sgRNA | | | | | | mUmCmUmACU*G*U*U |
| targeting | | | | | | *GUAGAUCACUUUCfCf |
| human | | | | | | UfUfAfGfUfGfC*fGfC*fA |
| albumin | | | | | | *fA*mA |
| MG29-1 | — | 5718 | hH29-4_37b | Nucleotide | N.A. | mC*mU*mU*U*UAAUU |
| sgRNA | | | | | | mUmCmUmACU*G*U*U |
| targeting | | | | | | *GUAGAUCCCCAGAfCf |
| human | | | | | | CfUfGfUfAfAfU*fAfG*fU |
| HA01 | | | | | | *fC*mA |
| MG29-1 | — | 5719 | hH29-21 37b | Nucleotide | N.A. | mC*mU*mU*U*UAAUU |
| sgRNA | | | | | | mUmCmUmACU*G*U*U |
| targeting | | | | | | *GUAGAUGGACAGAfGf |
| human | | | | | | GfGfUfCfAfGfC*fAfU*fG |
| HA01 | | | | | | *fC*mC |
| MG29-1 | — | 5720 | hH29-23_37b | Nucleotide | N.A. | mC*mU*mU*U*UAAUU |
| sgRNA | | | | | | mUmCmUmACU*G*U*U |
| targeting | | | | | | *GUAGAUUCAGCCUfGf |
| human | | | | | | UfCfAfGfUfCfC*fCfU*fG |
| HA01 | | | | | | *fG*mG |
| MG29-1 | — | 5721 | hH29-41_37b | Nucleotide | N.A. | mC*mU*mU*U*UAAUU |
| sgRNA | | | | | | mUmCmUmACU*G*U*U |
| targeting | | | | | | *GUAGAUAUAUCUUfCf |
| human | | | | | | CfCfAfGfCfUfG*fAfU*fA |
| HAO1 | | | | | | *fG*mA |
| MG29-1 | — | 5722 | hH29-4_50 | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| human | | | | | | *UAAUUmUmCmUmACU |
| HA01 | | | | | | *G*U*U*GUAGAUCCCC |
| | | | | | | AGAfCfCfUfGfUfAfAfUfA |
| | | | | | | fG*fU*fCfA*fU*mA |
| MG29-1 | — | 5723 | hH29-21_50 | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| human | | | | | | *UAAUUmUmCmUmACU |
| HA01 | | | | | | *G*U*U*GUAGAUGGAC |
| | | | | | | AGAfGfGfGfUfCfAfGfCf |
| | | | | | | AfU*fG*fCfC*fA*mA |
| MG29-1 | — | 5724 | hH29-23_50 | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| human | | | | | | *UAAUUmUmCmUmACU |
| HA01 | | | | | | *G*U*U*GUAGAUUCAG |
| | | | | | | CCUfGfUfCfAfGfUfCfCfC |
| | | | | | | fU*fG*fGfG*fA*mA |
| MG29-1 | — | 5725 | hH29-41_50 | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| human | | | | | | *UAAUUmUmCmUmACU |
| HA01 | | | | | | *G*U*U*GUAGAUAUAU |
| | | | | | | CUUfCfCfCfAfGfCfUfGfA |
| | | | | | | fU*fA*fGfA*fU*mG |
| MG29-1 | — | 5726 | hH29-4_50b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| human | | | | | | *UAAUUmUmCmUmACU |
| HA01 | | | | | | *G*U*U*GUAGAUCCCC |
| | | | | | | AGAfCfCfUfGfUfAfAfU*f |
| | | | | | | AfG*fU*fC*mA |
| MG29-1 | — | 5727 | hH29-21_50b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| human | | | | | | *UAAUUmUmCmUmACU |
| HA01 | | | | | | *G*U*U*GUAGAUGGAC |
| | | | | | | AGAfGfGfGfUfCfAfGfC*f |
| | | | | | | AfU*fG*fC*mC |
| MG29-1 | — | 5728 | hH29-23_50b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| human | | | | | | *UAAUUmUmCmUmACU |
| HA01 | | | | | | *G*U*U*GUAGAUUCAG |
| | | | | | | CCUfGfUfCfAfGfUfCfC*f |
| | | | | | | CfU*fG*fG*mG |
| MG29-1 | — | 5729 | hH29-41_50b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| human | | | | | | *UAAUUmUmCmUmACU |
| HA01 | | | | | | *G*U*U*GUAGAUAUAU |
| | | | | | | CUUfCfCfCfAfGfCfUfG*f |
| | | | | | | AfU*fA*fG*mA |
| MG29-1 | — | 5730 | mH29-1-50 | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HA01 | | | | | | *G*U*U*GUAGAUCCCC |
| | | | | | | AGAfCfCfUfGfUfAfAfUfA |
| | | | | | | fG*fU*fCfA*fU*mA |
| MG29-1 | — | 5731 | mH29-15-50 | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HA01 | | | | | | *G*U*U*GUAGAUUGAC |
| | | | | | | UGUfGfGfAfCfAfCfCfCf |
| | | | | | | CfU*fU*fAfC*fC*mU |
| MG29-1 | — | 5732 | mH29-1-50b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HA01 | | | | | | *G*U*U*GUAGAUCCCC |
| | | | | | | AGAfCfCfUfGfUfAfAfUfA |
| | | | | | | fG*fU*fC*mA |
| MG29-1 | — | 5733 | mH29-15-50b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HA01 | | | | | | *G*U*U*GUAGAUUGAC |
| | | | | | | UGUfGfGfAfCfAfCfCfCf |
| | | | | | | CfU*fU*fA*mC |
| SpCas9 | — | 5734 | mAlbR1 guide | Nucleotide | N.A. | mU*mU*mA*GUAUAGC |
| sgRNA | | | RNA | | | AUGGUCGAGCGUUUU |
| targeting | | | | | | AGAGCUAGAAAUAGCA |
| mouse | | | | | | AGUUAAAAUAAGGCUA |
| albumin | | | | | | GUCCGUUAUCAACUUG |
| | | | | | | AAAAAGUGGCACCGA |
| | | | | | | GUCGGUGCmU*mU*mU |
| | | | | | | *U |
| SpCas9 | — | 5735 | mAlbR2 guide | Nucleotide | N.A. | mU*mU*mC*CUGUAAC |
| sgRNA | | | RNA | | | GAUCGGGAACGUUUU |
| targeting | | | | | | AGAGCUAGAAAUAGCA |
| mouse | | | | | | AGUUAAAAUAAGGCUA |
| albumin | | | | | | GUCCGUUAUCAACUUG |
| | | | | | | AAAAAGUGGCACCGA |
| | | | | | | GUCGGUGCmU*mU*mU |
| | | | | | | *U |
| SpCas9 | — | 5736 | mAlbR3 guide | Nucleotide | N.A. | mU*mG*mC*CAGUUCC |
| sgRNA | | | RNA | | | CGAUCGUUACGUUUUA |
| targeting | | | | | | GAGCUAGAAAUAGCAA |
| mouse | | | | | | GUUAAAAUAAGGCUAG |
| albumin | | | | | | UCCGUUAUCAACUUGA |
| | | | | | | AAAAGUGGCACCGAG |
| | | | | | | UCGGUGCmU*mU*mU* |
| | | | | | | U |
| PCR | — | 5737 | mAlb90F PCR | Nucleotide | N.A. | CTCCTCTTCGTCTCCG |
| primer | | | primer | | | GC |
| PCR | — | 5738 | mAlb1073R PCR | Nucleotide | N.A. | CTGCCACATTGCTCAG |
| primer | | | primer | | | CAC |
| PCR | — | 5739 | mAlb282F PCR | Nucleotide | N.A. | TTGCATCTGAGAACCC |
| primer | | | primer | | | TTAGG |
| PCR | — | 5740 | mAlb460F PCR | Nucleotide | N.A. | GCCTGCTCGACCATGC |
| primer | | | primer | | | TATA |
| SpCas9 | — | 5741 | mAlbR2 guide | Nucleotide | N.A. | mU*mU*mC*CUGUAAC |
| sgRNA | | | RNA with | | | GAUCGGGAACGUUUU |
| targeting | | | extensive | | | AGAmGmCmUmAGAAA |
| mouse | | | chemical | | | mUmAmGmCAAGUUAA |
| albumin | | | modifications | | | AAUAAGGCUAGUCCGU |
| | | | | | | UAUCmAmAmCmUmUG |
| | | | | | | AAAmAmAmGmUmGmG |
| | | | | | | mCmAmCmCmGmAmG |
| | | | | | | mUmCmGmGmUmGmC |
| | | | | | | mU*mU*mU*U |
| SpCas9 | — | 5742 | Template DNA for | Nucleotide | N.A. | GCGGCCGCTAATACGA |
| DNA | | | spCas9 in vitro | | | CTCACTATAAGAAAAG |
| sequence | | | transcription | | | CCAGCTCCAGCAGGCG |
| | | | | | | CTGCTCACTCCTCCCC |
| | | | | | | ATCCTCTCCCTCTGTC |
| | | | | | | CCTCTGTCCCTCTGAC |
| | | | | | | CCTGCACTGTCCCAGC |
| | | | | | | ACCATGGCCCCCAAGA |
| | | | | | | AGAAGCGGAAAGTTG |
| | | | | | | GCGGCGGAGGCAGCG |
| | | | | | | ACAAGAAGTACTCTAT |
| | | | | | | CGGCCTGGACATCGGC |
| | | | | | | ACCAACTCTGTTGGAT |
| | | | | | | GGGCCGTGATCACCGA |
| | | | | | | CGAGTACAAGGTGCCC |
| | | | | | | AGCAAGAAATTCAAGG |
| | | | | | | TGCTGGGCAACACCGA |
| | | | | | | CCGGCACAGCATCAAG |
| | | | | | | AAGAATCTGATCGGCG |
| | | | | | | CCCTGCTGTTCGACTC |
| | | | | | | TGGCGAAACAGCCGAA |
| | | | | | | GCCACCAGACTGAAGA |
| | | | | | | GAACCGCCAGACGGC |
| | | | | | | GGTACACCAGAAGAAA |
| | | | | | | GAACCGGATCTGCTAC |
| | | | | | | CTGCAAGAGATCTTCA |
| | | | | | | GCAACGAGATGGCCAA |
| | | | | | | GGTGGACGACAGCTTC |
| | | | | | | TTCCACAGACTGGAAG |
| | | | | | | AGTCCTTCCTGGTGGA |
| | | | | | | AGAGGACAAGAAGCA |
| | | | | | | CGAGCGGCACCCCATC |
| | | | | | | TTCGGCAACATCGTGG |
| | | | | | | ATGAGGTGGCCTACCA |
| | | | | | | CGAGAAGTACCCCACC |
| | | | | | | ATCTACCACCTGAGAA |
| | | | | | | AGAAACTGGTGGACAG |
| | | | | | | CACCGACAAGGCCGAC |
| | | | | | | CTGAGACTGATCTATC |
| | | | | | | TGGCCCTGGCTCACAT |
| | | | | | | GATCAAGTTCCGGGGC |
| | | | | | | CACTTCCTGATCGAGG |
| | | | | | | GCGACCTGAATCCTGA |
| | | | | | | CAACAGCGACGTGGAC |
| | | | | | | AAGCTGTTCATCCAGC |
| | | | | | | TGGTGCAGACCTACAA |
| | | | | | | CCAGCTGTTCGAGGAA |
| | | | | | | AACCCCATCAACGCCA |
| | | | | | | GCGGAGTGGATGCCA |
| | | | | | | AGGCCATCCTGTCTGC |
| | | | | | | CAGACTGAGCAAGAGC |
| | | | | | | AGACGGCTGGAAAACC |
| | | | | | | TGATCGCTCAGCTGCC |
| | | | | | | CGGCGAGAAGAAGAA |
| | | | | | | TGGCCTGTTCGGCAAC |
| | | | | | | CTGATTGCCCTGAGCC |
| | | | | | | TGGGCCTGACACCTAA |
| | | | | | | CTTCAAGAGCAACTTC |
| | | | | | | GACCTGGCCGAGGAC |
| | | | | | | GCCAAACTGCAGCTGT |
| | | | | | | CCAAGGACACCTACGA |
| | | | | | | CGACGACCTGGACAAT |
| | | | | | | CTGCTGGCCCAGATCG |
| | | | | | | GCGATCAGTACGCCGA |
| | | | | | | CTTGTTTCTGGCCGCC |
| | | | | | | AAGAACCTGTCCGACG |
| | | | | | | CCATCCTGCTGAGCGA |
| | | | | | | CATCCTGAGAGTGAAC |
| | | | | | | ACCGAGATCACAAAGG |
| | | | | | | CCCCTCTGAGCGCCTC |
| | | | | | | TATGATCAAGAGATAC |
| | | | | | | GACGAGCACCACCAG |
| | | | | | | GATCTGACCCTGCTGA |
| | | | | | | AGGCCCTCGTTAGACA |
| | | | | | | GCAGCTGCCTGAGAAG |
| | | | | | | TACAAAGAGATTTTCT |
| | | | | | | TCGACCAGAGCAAGAA |
| | | | | | | CGGCTACGCCGGCTAC |
| | | | | | | ATTGATGGCGGAGCCA |
| | | | | | | GCCAAGAGGAATTCTA |
| | | | | | | CAAGTTCATCAAGCCC |
| | | | | | | ATCCTCGAGAAGATGG |
| | | | | | | ACGGCACCGAGGAACT |
| | | | | | | GCTGGTCAAGCTGAAC |
| | | | | | | AGAGAGGACCTGCTGC |
| | | | | | | GGAAGCAGCGGACCTT |
| | | | | | | CGACAATGGCTCTATC |
| | | | | | | CCTCACCAGATCCACC |
| | | | | | | TGGGAGAGCTGCACG |
| | | | | | | CCATTCTGCGGAGACA |
| | | | | | | AGAGGACTTTTACCCA |
| | | | | | | TTCCTGAAGGACAACC |
| | | | | | | GGGAAAAGATTGAGAA |
| | | | | | | GATCCTGACCTTCAGG |
| | | | | | | ATCCCCTACTACGTGG |
| | | | | | | GACCACTGGCCAGAG |
| | | | | | | GCAATAGCAGATTCGC |
| | | | | | | CTGGATGACCAGAAAG |
| | | | | | | AGCGAGGAAACCATCA |
| | | | | | | CACCCTGGAACTTCGA |
| | | | | | | GGAAGTGGTGGACAA |
| | | | | | | GGGCGCCAGCGCTCA |
| | | | | | | GTCCTTCATCGAGCGG |
| | | | | | | ATGACCAACTTCGATA |
| | | | | | | AGAACCTGCCTAACGA |
| | | | | | | GAAGGTGCTGCCCAAG |
| | | | | | | CACAGCCTGCTGTACG |
| | | | | | | AGTACTTCACCGTGTA |
| | | | | | | CAACGAGCTGACCAAA |
| | | | | | | GTGAAATACGTGACCG |
| | | | | | | AGGGAATGAGAAAGC |
| | | | | | | CCGCCTTTCTGAGCGG |
| | | | | | | CGAGCAGAAAAAGGC |
| | | | | | | CATTGTGGATCTGCTG |
| | | | | | | TTCAAGACCAACCGGA |
| | | | | | | AAGTGACCGTGAAGCA |
| | | | | | | GCTGAAAGAGGACTAC |
| | | | | | | TTCAAGAAAATCGAGT |
| | | | | | | GCTTCGACAGCGTGGA |
| | | | | | | AATCAGCGGCGTGGAA |
| | | | | | | GATCGGTTCAATGCCA |
| | | | | | | GCCTGGGCACATACCA |
| | | | | | | CGACCTGCTGAAAATT |
| | | | | | | ATCAAGGACAAGGACT |
| | | | | | | TCCTGGACAACGAAGA |
| | | | | | | GAACGAGGACATCCTG |
| | | | | | | GAAGATATCGTGCTGA |
| | | | | | | CCCTGACACTGTTTGA |
| | | | | | | GGACAGAGAGATGATC |
| | | | | | | GAGGAACGGCTGAAA |
| | | | | | | ACATACGCCCACCTGT |
| | | | | | | TCGACGACAAAGTGAT |
| | | | | | | GAAGCAACTGAAGCG |
| | | | | | | GCGGAGATACACCGG |
| | | | | | | CTGGGGCAGACTGTCT |
| | | | | | | CGGAAGCTGATCAACG |
| | | | | | | GCATCCGGGATAAGCA |
| | | | | | | GTCCGGCAAGACCATC |
| | | | | | | CTGGACTTTCTGAAGT |
| | | | | | | CCGACGGCTTCGCCAA |
| | | | | | | CAGAAACTTCATGCAG |
| | | | | | | CTGATCCACGACGACA |
| | | | | | | GCCTGACCTTTAAAGA |
| | | | | | | GGATATCCAGAAAGCC |
| | | | | | | CAGGTGTCCGGCCAG |
| | | | | | | GGCGATTCTCTGCATG |
| | | | | | | AGCACATTGCCAACCT |
| | | | | | | GGCCGGCTCTCCCGCC |
| | | | | | | ATTAAGAAGGGCATTC |
| | | | | | | TGCAGACAGTGAAGGT |
| | | | | | | GGTGGACGAGCTGGT |
| | | | | | | CAAAGTCATGGGCAGA |
| | | | | | | CACAAGCCCGAGAACA |
| | | | | | | TCGTGATCGAAATGGC |
| | | | | | | CAGAGAGAACCAGACC |
| | | | | | | ACACAGAAGGGCCAG |
| | | | | | | AAGAACAGCCGCGAG |
| | | | | | | AGAATGAAGCGGATCG |
| | | | | | | AAGAGGGCATCAAAGA |
| | | | | | | GCTGGGCAGCCAGATC |
| | | | | | | CTGAAAGAACACCCCG |
| | | | | | | TGGAAAACACCCAGCT |
| | | | | | | GCAGAACGAGAAGCT |
| | | | | | | GTACCTGTACTACCTC |
| | | | | | | CAGAACGGCCGGGAT |
| | | | | | | ATGTACGTGGACCAAG |
| | | | | | | AGCTGGACATCAACCG |
| | | | | | | GCTGTCCGACTACGAT |
| | | | | | | GTGGACCATATCGTGC |
| | | | | | | CCCAGTCTTTTCTGAA |
| | | | | | | GGACGACTCCATCGAC |
| | | | | | | AACAAGGTCCTGACCA |
| | | | | | | GATCCGACAAGAATCG |
| | | | | | | GGGCAAGAGCGACAA |
| | | | | | | CGTGCCCTCCGAAGAG |
| | | | | | | GTGGTCAAGAAGATGA |
| | | | | | | AGAACTACTGGCGACA |
| | | | | | | GCTGCTGAACGCCAAG |
| | | | | | | CTGATTACCCAGCGGA |
| | | | | | | AGTTCGATAACCTGAC |
| | | | | | | CAAGGCCGAGAGAGG |
| | | | | | | CGGCCTGTCTGAACTG |
| | | | | | | GATAAGGCCGGCTTCA |
| | | | | | | TCAAGAGACAGCTGGT |
| | | | | | | GGAAACCCGGCAGATC |
| | | | | | | ACCAAACACGTGGCAC |
| | | | | | | AGATTCTGGACTCCCG |
| | | | | | | GATGAACACTAAGTAC |
| | | | | | | GACGAGAATGACAAGC |
| | | | | | | TGATCCGGGAAGTGAA |
| | | | | | | AGTGATCACCCTGAAG |
| | | | | | | TCCAAGCTGGTGTCCG |
| | | | | | | ATTTCCGGAAGGATTT |
| | | | | | | CCAGTTCTACAAAGTG |
| | | | | | | CGCGAGATCAACAACT |
| | | | | | | ACCATCACGCCCACGA |
| | | | | | | CGCCTACCTGAATGCC |
| | | | | | | GTTGTTGGAACAGCCC |
| | | | | | | TGATCAAGAAGTATCC |
| | | | | | | CAAGCTGGAAAGCGA |
| | | | | | | GTTCGTGTACGGCGAC |
| | | | | | | TACAAGGTGTACGACG |
| | | | | | | TGCGGAAGATGATCGC |
| | | | | | | CAAGAGCGAGCAAGA |
| | | | | | | GATTGGCAAGGCTACC |
| | | | | | | GCCAAGTACTTTTTCT |
| | | | | | | ACAGCAACATCATGAA |
| | | | | | | CTTTTTCAAGACCGAG |
| | | | | | | ATTACCCTGGCCAACG |
| | | | | | | GCGAGATCAGAAAGC |
| | | | | | | GGCCTCTGATCGAGAC |
| | | | | | | AAACGGCGAAACCGG |
| | | | | | | CGAGATTGTGTGGGAT |
| | | | | | | AAGGGCAGAGACTTTG |
| | | | | | | CCACAGTGCGGAAAGT |
| | | | | | | GCTGAGCATGCCCCAA |
| | | | | | | GTGAATATCGTGAAGA |
| | | | | | | AAACCGAGGTGCAGAC |
| | | | | | | AGGCGGCTTCAGCAAA |
| | | | | | | GAGTCCATTCTGCCCA |
| | | | | | | AGAGAAACAGCGATAA |
| | | | | | | GCTGATCGCCCGGAAG |
| | | | | | | AAGGACTGGGACCCTA |
| | | | | | | AGAAGTACGGCGGCTT |
| | | | | | | CGATAGCCCTACCGTG |
| | | | | | | GCCTATTCTGTGCTGG |
| | | | | | | TGGTGGCCAAAGTGGA |
| | | | | | | AAAGGGCAAGTCCAAG |
| | | | | | | AAACTCAAGAGCGTGA |
| | | | | | | AAGAGCTGCTGGGGAT |
| | | | | | | CACCATCATGGAAAGA |
| | | | | | | AGCAGCTTCGAGAAGA |
| | | | | | | ATCCTATCGATTTCCT |
| | | | | | | CGAGGCCAAGGGCTA |
| | | | | | | CAAAGAAGTGAAAAAG |
| | | | | | | GACCTGATCATCAAGC |
| | | | | | | TCCCCAAGTACTCCCT |
| | | | | | | GTTCGAGCTGGAAAAT |
| | | | | | | GGCCGGAAGCGGATG |
| | | | | | | CTGGCTTCTGCTGGCG |
| | | | | | | AACTGCAGAAGGGAAA |
| | | | | | | CGAACTGGCCCTGCCT |
| | | | | | | AGCAAATATGTGAACT |
| | | | | | | TCCTGTACCTGGCCAG |
| | | | | | | CCACTATGAGAAGCTG |
| | | | | | | AAGGGCAGCCCCGAG |
| | | | | | | GACAATGAGCAAAAGC |
| | | | | | | AGCTGTTTGTGGAACA |
| | | | | | | GCACAAGCACTACCTG |
| | | | | | | GACGAGATCATCGAGC |
| | | | | | | AGATCAGCGAGTTCTC |
| | | | | | | CAAGAGAGTGATCCTG |
| | | | | | | GCCGACGCTAATCTGG |
| | | | | | | ACAAAGTGCTGTCCGC |
| | | | | | | CTACAACAAGCACCGG |
| | | | | | | GACAAGCCTATCAGAG |
| | | | | | | AGCAGGCCGAGAATAT |
| | | | | | | CATCCACCTGTTTACC |
| | | | | | | CTGACCAATCTGGGAG |
| | | | | | | CCCCTGCCGCCTTCAA |
| | | | | | | GTACTTCGACACCACC |
| | | | | | | ATCGACCGGAAGCGCT |
| | | | | | | ACACCAGCACCAAAGA |
| | | | | | | GGTGCTGGACGCCACA |
| | | | | | | CTGATCCACCAGTCTA |
| | | | | | | TCACCGGCCTGTACGA |
| | | | | | | GACACGGATCGACCTG |
| | | | | | | TCTCAGCTCGGAGGCG |
| | | | | | | ATTCTGGCGGAAAAAG |
| | | | | | | ACCTGCCGCCACAAAG |
| | | | | | | AAAGCCGGACAGGCC |
| | | | | | | AAGAAAAAGAAGTGAC |
| | | | | | | CACACCCCCATTCCCC |
| | | | | | | CACTCCAGATAAAGCT |
| | | | | | | TCAGTTATATCTCACG |
| | | | | | | TGTCTGGAGTTAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAAA |
| | | | | | | AAAAAAAAAAAAAAAG |
| | | | | | | AAGAGCCCTGCAGG |
| SpCas9 | — | 5743 | Amino acid | Protein | N.A. | MAPKKKRKVGGGGSD |
| protein | | | sequence of | | | KKYSIGLDIGTNSVGWA |
| sequence | | | spCas9 encoded in | | | VITDEYKVPSKKFKVLG |
| | | | mRNA inclusing | | | NTDRHSIKKNLIGALLF |
| | | | nuclear | | | DSGETAEATRLKRTAR |
| | | | localization | | | RRYTRRKNRICYLQEIF |
| | | | signals | | | SNEMAKVDDSFFHRLE |
| | | | | | | ESFLVEEDKKHERHPIF |
| | | | | | | GNIVDEVAYHEKYPTIY |
| | | | | | | HLRKKLVDSTDKADLR |
| | | | | | | LIYLALAHMIKFRGHFL |
| | | | | | | IEGDLNPDNSDVDKLFI |
| | | | | | | QLVQTYNQLFEENPINA |
| | | | | | | SGVDAKAILSARLSKSR |
| | | | | | | RLENLIAQLPGEKKNGL |
| | | | | | | FGNLIALSLGLTPNFKS |
| | | | | | | NFDLAEDAKLQLSKDT |
| | | | | | | YDDDLDNLLAQIGDQY |
| | | | | | | ADLFLAAKNLSDAILLS |
| | | | | | | DILRVNTEITKAPLSAS |
| | | | | | | MIKRYDEHHQDLTLLK |
| | | | | | | ALVRQQLPEKYKEIFFD |
| | | | | | | QSKNGYAGYIDGGASQ |
| | | | | | | EEFYKFIKPILEKMDGT |
| | | | | | | EELLVKLNREDLLRKQ |
| | | | | | | RTFDNGSIPHQIHLGEL |
| | | | | | | HAILRRQEDFYPFLKDN |
| | | | | | | REKIEKILTFRIPYYVGP |
| | | | | | | LARGNSRFAWMTRKSE |
| | | | | | | ETITPWNFEEVVDKGAS |
| | | | | | | AQSFIERMTNFDKNLPN |
| | | | | | | EKVLPKHSLLYEYFTVY |
| | | | | | | NELTKVKYVTEGMRKP |
| | | | | | | AFLSGEQKKAIVDLLFK |
| | | | | | | TNRKVTVKQLKEDYFK |
| | | | | | | KIECFDSVEISGVEDRFN |
| | | | | | | ASLGTYHDLLKIIKDKD |
| | | | | | | FLDNEENEDILEDIVLTL |
| | | | | | | TLFEDREMIEERLKTYA |
| | | | | | | HLFDDKVMKQLKRRR |
| | | | | | | YTGWGRLSRKLINGIRD |
| | | | | | | KQSGKTILDFLKSDGFA |
| | | | | | | NRNFMQLIHDDSLTFKE |
| | | | | | | DIQKAQVSGQGDSLHE |
| | | | | | | HIANLAGSPAIKKGILQ |
| | | | | | | TVKVVDELVKVMGRH |
| | | | | | | KPENIVIEMARENQTTQ |
| | | | | | | KGQKNSRERMKRIEEG |
| | | | | | | IKELGSQILKEHPVENT |
| | | | | | | QLQNEKLYLYYLQNGR |
| | | | | | | DMYVDQELDINRLSDY |
| | | | | | | DVDHIVPQSFLKDDSID |
| | | | | | | NKVLTRSDKNRGKSDN |
| | | | | | | VPSEEVVKKMKNYWR |
| | | | | | | QLLNAKLITQRKFDNLT |
| | | | | | | KAERGGLSELDKAGFIK |
| | | | | | | RQLVETRQITKHVAQIL |
| | | | | | | DSRMNTKYDENDKLIR |
| | | | | | | EVKVITLKSKLVSDFRK |
| | | | | | | DFQFYKVREINNYHHA |
| | | | | | | HDAYLNAVVGTALIKK |
| | | | | | | YPKLESEFVYGDYKVY |
| | | | | | | DVRKMIAKSEQEIGKAT |
| | | | | | | AKYFFYSNIMNFFKTEI |
| | | | | | | TLANGEIRKRPLIETNG |
| | | | | | | ETGEIVWDKGRDFATV |
| | | | | | | RKVLSMPQVNIVKKTE |
| | | | | | | VQTGGFSKESILPKRNS |
| | | | | | | DKLIARKKDWDPKKYG |
| | | | | | | GFDSPTVAYSVLVVAKV |
| | | | | | | EKGKSKKLKSVKELLGI |
| | | | | | | TIMERSSFEKNPIDFLEA |
| | | | | | | KGYKEVKKDLIIKLPKY |
| | | | | | | SLFELENGRKRMLASA |
| | | | | | | GELQKGNELALPSKYV |
| | | | | | | NFLYLASHYEKLKGSPE |
| | | | | | | DNEQKQLFVEQHKHYL |
| | | | | | | DEIIEQISEFSKRVILAD |
| | | | | | | ANLDKVLSAYNKHRDK |
| | | | | | | PIREQAENIIHLFTLTNL |
| | | | | | | GAPAAFKYFDTTIDRKR |
| | | | | | | YTSTKEVLDATLIHQSIT |
| | | | | | | GLYETRIDLSQLGGDSG |
| | | | | | | GKRPAATKKAGQAKK |
| | | | | | | KK |
| MG29-1 | — | 5744 | mAlb29-8-50 | Nucleotide | N.A. | mG*mU*mU*GAGAAUC |
| sgRNA | | | guide RNA | | | *mG*mA*mA*mAGAUU |
| targeting | | | | | | CUCAAC*mC*mU*mU*U |
| mouse | | | | | | *UAAUUmUmCmUmACU |
| albumin | | | | | | *G*U*U*GUAGAUCUGU |
| | | | | | | AACfGfAfUfCfGfGfGfAf |
| | | | | | | AfC*fU*fGfG*fC*mA |
| MG29-1 | — | 5788 | hH29-1 | Nucleotide | N.A. | UAAUUUCUACUGUUGU |
| sgRNA | | | | | | AGAUGCAUGUUGUUCA |
| targeting | | | | | | UAAUCAUUGA |
| human | | | | | | |
| HAO-1 | | | | | | |
| MG29-1 | — | 5789 | hH29-2 | Nucleotide | N.A. | UAAUUUCUACUGUUGU |
| sgRNA | | | | | | AGAUGAAGUACUGAUU |
| targeting | | | | | | UAGCAUGUUG |
| human | | | | | | |
| HAO-1 | | | | | | |
| MG29-1 | — | 5790 | hH29-3 | Nucleotide | N.A. | UAAUUUCUACUGUUGU |
| sgRNA | | | | | | AGAUUAUCAAUGAUUA |
| targeting | | | | | | UGAACAACAU |
| human | | | | | | |
| HAO-1 | | | | | | |
| MG29-1 | — | 5791 | hH29-4 | Nucleotide | N.A. | UAAUUUCUACUGUUGU |
| sgRNA | | | | | | AGAUCCCCAGACCUGU |
| targeting | | | | | | AAUAGUCAUA |
| human | | | | | | |
| HAO-1 | | | | | | |
| MG29-1 | — | 5792 | hH29-5 | Nucleotide | N.A. | UAAUUUCUACUGUUGU |
| sgRNA | | | | | | AGAUUUCAUCAUUUGC |
| targeting | | | | | | CCCAGACCUG |
| human | | | | | | |
| HAO-1 | | | | | | |
| MG29-1 | — | 5793 | hH29-6 | Nucleotide | N.A. | UAAUUUCUACUGUUGU |
| sgRNA | | | | | | AGAUUUACCUGGAAAA |
| targeting | | | | | | UGCUGCAAUA |
| human | | | | | | |
| HAO-1 | | | | | | |
| MG29-1 | — | 5794 | hH29-7 | Nucleotide | N.A. | UAAUUUCUACUGUUGU |
| sgRNA | | | | | | AGAUCUUACCUGGAAA |
| targeting | | | | | | AUGCUGCAAU |
| human | | | | | | |
| HAO-1 | | | | | | |
| MG29-1 | — | 5795 | hH29-8 | Nucleotide | N.A. | UAAUUUCUACUGUUGU |
| sgRNA | | | | | | AGAUGCUGAUAAUAUU |
| targeting | | | | | | GCAGCAUUUU |
| human | | | | | | |
| HAO-1 | | | | | | |
| MG29-1 | — | 5796 | hH29-9 | Nucleotide | N.A. | UAAUUUCUACUGUUGU |
| sgRNA | | | | | | AGAUAAAAAUAAAUUU |
| targeting | | | | | | UCUUACCUGG |
| human | | | | | | |
| HAO-1 | | | | | | |
| MG29-1 | — | 5797 | hH29-10 | Nucleotide | N.A. | UAAUUUCUACUGUUGU |
| sgRNA | | | | | | AGAUAAAAAAUAAAUU |
| targeting | | | | | | UUCUUACCUG |
| human | | | | | | |
| HAO-1 | | | | | | |
| MG29-1 | — | 5798 | hH29-11 | Nucleotide | N.A. | UAAUUUCUACUGUUGU |
| sgRNA | | | | | | AGAUAUUUUAUUUUUU |
| targeting | | | | | | AAUUCUAGAU |
| human | | | | | | |
| HAO-1 | | | | | | |
| MG29-1 | — | 5799 | hH29-12 | Nucleotide | N.A | UAAUUUCUACUGUUGU |
| sgRNA | | | | | | AGAUUUUUAUUUUUUA |
| targeting | | | | | | AUUCUAGAUG |
| human | | | | | | |
| HAO-1 | | | | | | |
| MG29-1 | — | 5800 | hH29-13 | Nucleotide | N.A. | UAAUUUCUACUGUUGU |
| sgRNA | | | | | | AGAUAUUUUUUAAUUC |
| targeting | | | | | | UAGAUGGAAG |
| human | | | | | | |
| HAO-1 | | | | | | |
| MG29-1 | — | 5801 | hH29-14 | Nucleotide | N.A. | UAAUUUCUACUGUUGU |
| sgRNA | | | | | | AGAUUUUUUUAAUUCU |
| targeting | | | | | | AGAUGGAAGC |
| human | | | | | | |
| HAO-1 | | | | | | |
| MG29-1 | — | 5802 | hH29-15 | Nucleotide | N.A. | UAAUUUCUACUGUUGU |
| sgRNA | | | | | | AGAUUUAAUUCUAGAU |
| targeting | | | | | | GGAAGCUGUA |
| human | | | | | | |
| HAO-1 | | | | | | |
| MG29-1 | — | 5803 | hH29-16 | Nucleotide | N.A. | UAAUUUCUACUGUUGU |
| sgRNA | | | | | | AGAUUAAUUCUAGAUG |
| targeting | | | | | | GAAGCUGUAU |
| human | | | | | | |
| HAO-1 | | | | | | |
| MG29-1 | — | 5804 | hH29-17 | Nucleotide | N.A. | UAAUUUCUACUGUUGU |
| sgRNA | | | | | | AGAUAAUUCUAGAUGG |
| targeting | | | | | | AAGCUGUAUC |
| human | | | | | | |
| HAO-1 | | | | | | |
| MG29-1 | — | 5805 | hH29-18 | Nucleotide | N.A. | UAAUUUCUACUGUUGU |
| sgRNA | | | | | | AGAUAUUCUAGAUGGA |
| targeting | | | | | | AGCUGUAUCC |
| human | | | | | | |
| HAO-1 | | | | | | |
| MG29-1 | — | 5806 | hH29-19 | Nucleotide | N.A. | UAAUUUCUACUGUUGU |
| sgRNA | | | | | | AGAUAGCAACAUUCCG |
| targeting | | | | | | GAGCAUCCUU |
| human | | | | | | |
| HAO-1 | | | | | | |
| MG29-1 | — | 5807 | hH29-20 | Nucleotide | N.A. | UAAUUUCUACUGUUGU |
| sgRNA | | | | | | AGAUAGGACAGAGGG |
| targeting | | | | | | UCAGCAUGCCA |
| human | | | | | | |
| HAO-1 | | | | | | |
| MG29-1 | — | 5808 | hH29-21 | Nucleotide | N.A. | UAAUUUCUACUGUUGU |
| sgRNA | | | | | | AGAUGGACAGAGGGU |
| targeting | | | | | | CAGCAUGCCAA |
| human | | | | | | |
| HAO-1 | | | | | | |
| MG29-1 | — | 5809 | hH29-22 | Nucleotide | N.A. | UAAUUUCUACUGUUGU |
| sgRNA | | | | | | AGAUUUUCUCAGCCUG |
| targeting | | | | | | UCAGUCCCUG |
| human | | | | | | |
| HAO-1 | | | | | | |
| MG29-1 | — | 5810 | hH29-23 | Nucleotide | N.A. | UAAUUUCUACUGUUGU |
| sgRNA | | | | | | AGAUUCAGCCUGUCAG |
| targeting | | | | | | UCCCUGGGAA |
| human | | | | | | |
| HAO-1 | | | | | | |
| MG29-1 | — | 5811 | hH29-24 | Nucleotide | N.A. | UAAUUUCUACUGUUGU |
| sgRNA | | | | | | AGAUUGACAGUGGAC |
| targeting | | | | | | ACACCUUACCU |
| human | | | | | | |
| HAO-1 | | | | | | |
| MG29-1 | — | 5812 | hH29-25 | Nucleotide | N.A. | UAAUUUCUACUGUUGU |
| sgRNA | | | | | | AGAUAAUCUGUUACGC |
| targeting | | | | | | ACAUCAUCCA |
| human | | | | | | |
| HAO-1 | | | | | | |
| MG29-1 | — | 5813 | hH29-26 | Nucleotide | N.A. | UAAUUUCUACUGUUGU |
| sgRNA | | | | | | AGAUAUGCAUUUCUUA |
| targeting | | | | | | UUUUAGGAUG |
| human | | | | | | |
| HAO-1 | | | | | | |
| MG29-1 | — | 5814 | hH29-27 | Nucleotide | N.A. | UAAUUUCUACUGUUGU |
| sgRNA | | | | | | AGAUUGCAUUUCUUAU |
| targeting | | | | | | UUUAGGAUGA |
| human | | | | | | |
| HAO-1 | | | | | | |
| MG29-1 | — | 5815 | hH29-28 | Nucleotide | N.A. | UAAUUUCUACUGUUGU |
| sgRNA | | | | | | AGAUUUAUUUUAGGAU |
| targeting | | | | | | GAAAAAUUUU |
| human | | | | | | |
| HAO-1 | | | | | | |
| MG29-1 | — | 5816 | hH29-29 | Nucleotide | N.A. | UAAUUUCUACUGUUGU |
| sgRNA | | | | | | AGAUAGGAUGAAAAAU |
| targeting | | | | | | UUUGAAACCA |
| human | | | | | | |
| HAO-1 | | | | | | |
| MG29-1 | — | 5817 | hH29-30 | Nucleotide | N.A. | UAAUUUCUACUGUUGU |
| sgRNA | | | | | | AGAUGGAUGAAAAAUU |
| targeting | | | | | | UUGAAACCAG |
| human | | | | | | |
| HAO-1 | | | | | | |
| MG29-1 | — | 5818 | hH29-31 | Nucleotide | N.A. | UAAUUUCUACUGUUGU |
| sgRNA | | | | | | AGAUCUCAGGAGAAAA |
| targeting | | | | | | UGAUAAAGUA |
| human | | | | | | |
| HAO-1 | | | | | | |
| MG29-1 | — | 5819 | hH29-32 | Nucleotide | N.A. | UAAUUUCUACUGUUGU |
| sgRNA | | | | | | AGAUCCUCAGGAGAAA |
| targeting | | | | | | AUGAUAAAGU |
| human | | | | | | |
| HAO-1 | | | | | | |
| MG29-1 | — | 5820 | hH29-33 | Nucleotide | N.A. | UAAUUUCUACUGUUGU |
| sgRNA | | | | | | AGAUGAAACCAGUACU |
| targeting | | | | | | UUAUCAUUUU |
| human | | | | | | |
| HAO-1 | | | | | | |
| MG29-1 | — | 5821 | hH29-34 | Nucleotide | N.A. | UAAUUUCUACUGUUGU |
| sgRNA | | | | | | AGAUAAACCAGUACUU |
| targeting | | | | | | UAUCAUUUUC |
| human | | | | | | |
| HAO-1 | | | | | | |
| MG29-1 | | 5822 | hH29-35 | Nucleotide | N.A. | UAAUUUCUACUGUUGU |
| sgRNA | | | | | | AGAUUCAUUUUCUCCU |
| targeting | | | | | | GAGGAAAAUU |
| human | | | | | | |
| HAO-1 | | | | | | |
| MG29-1 | — | 5823 | hH29-36 | Nucleotide | N.A. | UAAUUUCUACUGUUGU |
| sgRNA | | | | | | AGAUCUCCUGAGGAAA |
| targeting | | | | | | AUUUUGGAGA |
| human | | | | | | |
| HAO-1 | | | | | | |
| MG29-1 | — | 5824 | hH29-37 | Nucleotide | N.A. | UAAUUUCUACUGUUGU |
| sgRNA | | | | | | AGAUUCCUGAGGAAAA |
| targeting | | | | | | UUUUGGAGAC |
| human | | | | | | |
| HAO-1 | | | | | | |
| MG29-1 | — | 5825 | hH29-38 | Nucleotide | N.A. | UAAUUUCUACUGUUGU |
| sgRNA | | | | | | AGAUGCCACAUAUGCA |
| targeting | | | | | | GCAAGUCCAC |
| human | | | | | | |
| HAO-1 | | | | | | |
| MG29-1 | — | 5826 | hH29-39 | Nucleotide | N.A. | UAAUUUCUACUGUUGU |
| sgRNA | | | | | | AGAUGGAGACGACAG |
| targeting | | | | | | UGGACUUGCUG |
| human | | | | | | |
| HAO-1 | | | | | | |
| MG29-1 | — | 5827 | hH29-40 | Nucleotide | N.A. | UAAUUUCUACUGUUGU |
| sgRNA | | | | | | AGAUGAGACGACAGU |
| targeting | | | | | | GGACUUGCUGC |
| human | | | | | | |
| HAO-1 | | | | | | |
| MG29-1 | — | 5828 | hH29-41 | Nucleotide | N.A. | UAAUUUCUACUGUUGU |
| sgRNA | | | | | | AGAUAUAUCUUCCCAG |
| targeting | | | | | | CUGAUAGAUG |
| human | | | | | | |
| HAO-1 | | | | | | |
| MG29-1 | — | 5829 | hH29-42 | Nucleotide | N.A. | UAAUUUCUACUGUUGU |
| sgRNA | | | | | | AGAUCAACAAUUGGCA |
| targeting | | | | | | AUGAUGUCAG |
| human | | | | | | |
| HAO-1 | | | | | | |
| DNA | — | 5830 | DNA sequence | Nucleotide | N.A. | AAAAGCCAGCTCCAGC |
| sequence | | | encoding the | | | AGGCGCTGCTCACTCC |
| encoding | | | MG29-1 | | | TCCCCATCCTCTCCCT |
| the | | | messenger RNA | | | CTGTCCCTCTGTCCCT |
| MG29-1 | | | | | | CTGACCCTGCACTGTC |
| messenger | | | | | | CCAGCACCATGGCCCC |
| RNA | | | | | | TAAGAAGAAGAGAAAA |
| | | | | | | GTCGGCGGAGGCGGC |
| | | | | | | AGCTTCAACAACTTCA |
| | | | | | | TCAAGAAGTACAGCCT |
| | | | | | | GCAGAAAACCCTGCGC |
| | | | | | | TTCGAGCTGAAGCCTG |
| | | | | | | TGGGCGAGACAGCCG |
| | | | | | | ACTACATCGAGGACTT |
| | | | | | | CAAGAGCGAGTACCTG |
| | | | | | | AAGGACACCGTGCTGA |
| | | | | | | AGGACGAGCAGAGAG |
| | | | | | | CCAAGGACTACCAAGA |
| | | | | | | GATCAAGACCCTGATC |
| | | | | | | GACGATTACCACCGCG |
| | | | | | | AGTACATCGAAGAGTG |
| | | | | | | CCTGAGAGAACCCGTG |
| | | | | | | GACAAGAAAACCGGC |
| | | | | | | GAGATCCTGGACTTCA |
| | | | | | | CCCAGGACCTGGAAGA |
| | | | | | | TGCCTTCAGCTACTAC |
| | | | | | | CAGAAGCTGAAAGAGA |
| | | | | | | ACCCCACCGAGAACAG |
| | | | | | | AGTCGGCTGGGAGAA |
| | | | | | | AGAGCAAGAGAGCCT |
| | | | | | | GAGGAAGAAGCTGGT |
| | | | | | | CACCTCCTTCGTGGGC |
| | | | | | | AACGACGGCCTGTTCA |
| | | | | | | AGAAAGAGTTCATCAC |
| | | | | | | CAGGGACCTGCCTGAG |
| | | | | | | TGGCTGCAGAAGAAAG |
| | | | | | | GACTCTGGGGCGAGTA |
| | | | | | | CAAGGACACAGTGGAA |
| | | | | | | AACTTCAAGAAGTTCA |
| | | | | | | CCACCTACTTCAGCGG |
| | | | | | | CTTCCACGAGAACCGG |
| | | | | | | AAGAACATGTACACCG |
| | | | | | | CCGAGGCTCAGAGCAC |
| | | | | | | CGCTATCGCCAACAGA |
| | | | | | | CTGATGAACGACAACC |
| | | | | | | TGCCTAAGTTCTTTAA |
| | | | | | | CAACTACCTGGCCTAC |
| | | | | | | CAGACCATCAAAGAGA |
| | | | | | | AGCACCCCGACCTGGT |
| | | | | | | GTTCAGACTGGATGAT |
| | | | | | | GCTCTGCTGCAGGCCG |
| | | | | | | CTGGCGTGGAACATCT |
| | | | | | | GGATGAGGCTTTCCAG |
| | | | | | | CCTAGATACTTCAGCA |
| | | | | | | GACTGTTCGCCCAGAG |
| | | | | | | CGGCATCACCGCTTTC |
| | | | | | | AACGAGCTGATCGGCG |
| | | | | | | GCAGAACCACAGAGAA |
| | | | | | | CGGCGAGAAGATCCA |
| | | | | | | GGGCCTGAACGAGCA |
| | | | | | | GATCAACCTGTACAGA |
| | | | | | | CAGCAGAACCCCGAGA |
| | | | | | | AGGCCAAGGGCTTCCC |
| | | | | | | CAGATTCATGCCTCTG |
| | | | | | | TTCAAGCAGATCCTGA |
| | | | | | | GCGACAGAGAGACAC |
| | | | | | | ACAGCTTTCTGCCCGA |
| | | | | | | CGCCTTCGAGAACGAC |
| | | | | | | AAAGAGCTGCTCCAGG |
| | | | | | | CTCTGAGAGACTACGT |
| | | | | | | GGACGCCGCCACATCT |
| | | | | | | GAGGAAGGCATGATCA |
| | | | | | | GCCAGCTGAACAAGGC |
| | | | | | | CATGAACCAGTTCGTG |
| | | | | | | ACCGCCGACCTGAAGA |
| | | | | | | GAGTGTACATCAAGAG |
| | | | | | | CGCCGCTCTGACCAGC |
| | | | | | | CTGAGCCAAGAGCTGT |
| | | | | | | TCCACTTCTTCGGCGT |
| | | | | | | GATCAGCGACGCTATC |
| | | | | | | GCTTGGTACGCCGAGA |
| | | | | | | AGAGACTGAGCCCCAA |
| | | | | | | GAAGGCCCAAGAGTCT |
| | | | | | | TTCCTGAAGCAAGAGG |
| | | | | | | TGTACGCCATCGAGGA |
| | | | | | | ACTGAACCAGGCTGTC |
| | | | | | | GTGGGCTACATCGACC |
| | | | | | | AGCTGGAAGATCAGAG |
| | | | | | | CGAGCTGCAGCAACTG |
| | | | | | | CTGGTGGACCTGCCAG |
| | | | | | | ATCCTCAGAAACCCGT |
| | | | | | | GTCCAGCTTCATCCTG |
| | | | | | | ACACACTGGCAGAAGT |
| | | | | | | CTCAAGAGCCCCTGCA |
| | | | | | | GGCAGTGATCGCCAAG |
| | | | | | | GTGGAACCTCTGTTCG |
| | | | | | | AACTGGAAGAACTGAG |
| | | | | | | CAAGAACAAGAGGGC |
| | | | | | | CCCAAAGCACGACAAG |
| | | | | | | GACCAAGGCGGCGAG |
| | | | | | | GGATTTCAGCAGGTCG |
| | | | | | | ACGCCATCAAGAACAT |
| | | | | | | GCTGGACGCCTTCATG |
| | | | | | | GAAGTGTCCCACGCTA |
| | | | | | | TCAAGCCCCTGTACCT |
| | | | | | | GGTCAAGGGAAGAAA |
| | | | | | | GGCCATCGACATGCCC |
| | | | | | | GACGTGGACACCGGCT |
| | | | | | | TCTACGCTGATTTCGC |
| | | | | | | CGAGGCCTACAGCGCC |
| | | | | | | TACGAGCAAGTGACAG |
| | | | | | | TGTCCCTGTACAACAA |
| | | | | | | GACCAGAAACCACCTG |
| | | | | | | TCCAAGAAGCCCTTCA |
| | | | | | | GCAAGGACAAGATCAA |
| | | | | | | GATCAACTTCGACGCC |
| | | | | | | CCTACACTGCTGAACG |
| | | | | | | GCTGGGACCTGAACAA |
| | | | | | | AGAGAGCGACAACAA |
| | | | | | | GTCCATCATCCTGCGG |
| | | | | | | AAGGACGGCAACTTCT |
| | | | | | | ACCTGGCAATCATGCA |
| | | | | | | CCCCAAGCACACCAAG |
| | | | | | | GTGTTCGACTGCTACT |
| | | | | | | CTGCCTCTGAGGCTGC |
| | | | | | | CGGCAAGTGCTACGAG |
| | | | | | | AAGATGAACTACAAGC |
| | | | | | | TGCTGAGCGGCGCCAA |
| | | | | | | CAAGATGCTGCCTAAG |
| | | | | | | GTGTTCTTTAGCAAGA |
| | | | | | | AGGGCATCGAGACATT |
| | | | | | | CAGCCCTCCACAAGAA |
| | | | | | | ATCCTGGACCTGTACA |
| | | | | | | AGAACAACGAGCATAA |
| | | | | | | GAAGGGCGCCACCTTC |
| | | | | | | AAGCTGGAATCCTGCC |
| | | | | | | ACAAGCTGATCGATTT |
| | | | | | | CTTCAAGCGGAACATC |
| | | | | | | CCCAAGTACAAGGTGC |
| | | | | | | ACCCTACCGACAACTT |
| | | | | | | TGGCTGGGACGTGTTC |
| | | | | | | GGCTTTCACTTCAGCC |
| | | | | | | CTACCAGCAGCTACGG |
| | | | | | | CGACCTGTCTGGCTTC |
| | | | | | | TACAGAGAGGTGGAA |
| | | | | | | GCCCAGGGATACAAGC |
| | | | | | | TGTGGTTCAGCGACGT |
| | | | | | | GTCCGAGGCTTACATC |
| | | | | | | AACAAATGCGTGGAAG |
| | | | | | | AGGGCAAGCTGTTCCT |
| | | | | | | GTTCCAAATCTACAAC |
| | | | | | | AAGGACTTCTCCCCTA |
| | | | | | | ACTCCACCGGCAAGCC |
| | | | | | | CAACCTGCACACCCTG |
| | | | | | | TATTGGAAGGGCCTGT |
| | | | | | | TCGAGCCCGAGAACCT |
| | | | | | | GAAAGACGTGGTGCTG |
| | | | | | | AAGCTGAATGGCGAG |
| | | | | | | GCCGAGATCTTCTACC |
| | | | | | | GGAAGCACAGCATCAA |
| | | | | | | GCACGAGGACAAGAC |
| | | | | | | CATCCACAGAGCTAAG |
| | | | | | | GACCCTATCGCTAACA |
| | | | | | | AGAACGCTGACAACCC |
| | | | | | | CAAGAAACAGAGCGTG |
| | | | | | | TTCGATTACGACATCA |
| | | | | | | TCAAGGATAAGCGGTA |
| | | | | | | TACCCAGGACAAGTTC |
| | | | | | | TTCTTCCACGTGCCAA |
| | | | | | | TCAGCCTGAACTTCAA |
| | | | | | | AAGCCAGGGCGTCGT |
| | | | | | | GCGGTTCAACGATAAG |
| | | | | | | ATCAACGGCCTGCTGG |
| | | | | | | CCGCTCAGGACGATGT |
| | | | | | | GCATGTGATCGGCATC |
| | | | | | | GACAGAGGCGAGAGA |
| | | | | | | CATCTGCTGTACTACA |
| | | | | | | CCGTGGTCAACGGCAA |
| | | | | | | GGGCGAAGTGGTGGA |
| | | | | | | ACAGGGCAGCCTGAAT |
| | | | | | | CAGGTGGCCACAGATC |
| | | | | | | AGGGCTACGTGGTGG |
| | | | | | | ATTACCAGCAGAAGCT |
| | | | | | | GCACGCCAAAGAGAAA |
| | | | | | | GAACGCGACCAGGCC |
| | | | | | | AGAAAGAACTGGTCCA |
| | | | | | | CCATCGAGAACATCAA |
| | | | | | | AGAACTGAAGGCCGG |
| | | | | | | CTACCTGAGCCAGGTG |
| | | | | | | GTGCATAAGCTGGCTC |
| | | | | | | AGCTGATCGTGAAGCA |
| | | | | | | CAACGCCATCGTGTGC |
| | | | | | | CTCGAGGACCTGAATT |
| | | | | | | TCGGCTTCAAGAGGGG |
| | | | | | | CAGATTCAAGGTCGAG |
| | | | | | | AAACAGGTGTACCAGA |
| | | | | | | AGTTCGAGAAGGCTCT |
| | | | | | | GATCGACAAGCTGAAC |
| | | | | | | TACCTCGTGTTCAAAG |
| | | | | | | AGAGAGGCGCCACAC |
| | | | | | | AGGCTGGCGGATACCT |
| | | | | | | GAATGCTTACCAGCTG |
| | | | | | | GCCGCACCTTTCGAGA |
| | | | | | | GCTTTGAGAAGCTGGG |
| | | | | | | CAAGCAGACCGGCATC |
| | | | | | | CTGTACTACGTGCGGA |
| | | | | | | GCGACTACACCAGCAA |
| | | | | | | GATCGACCCTGCTACC |
| | | | | | | GGCTTCGTGGACTTTC |
| | | | | | | TGAAGCCTAAGTACGA |
| | | | | | | GAGCATGGCCAAGAG |
| | | | | | | CAAAGTGTTCTTCGAG |
| | | | | | | TCCTTCGAGCGCATCC |
| | | | | | | AGTGGAACCAGGCCAA |
| | | | | | | AGGCTACTTCGAGTTC |
| | | | | | | GAGTTTGACTACAAGA |
| | | | | | | AGATGTGCCCCAGCAG |
| | | | | | | AAAGTTCGGCGACTAC |
| | | | | | | AGAACCAGATGGGTCG |
| | | | | | | TGTGCACCTTCGGCGA |
| | | | | | | CACCCGCTACCAGAAC |
| | | | | | | AGAAGAAACAAGAGCA |
| | | | | | | GCGGCCAGTGGGAGA |
| | | | | | | CAGAGACAATCGATGT |
| | | | | | | GACAGCCCAGCTGAAA |
| | | | | | | GCCCTGTTCGCCGCTT |
| | | | | | | ACGGCATCACATACAA |
| | | | | | | TCAAGAGGATAACATC |
| | | | | | | AAGGACGCCATTGCCG |
| | | | | | | CCGTGAAGTACACCAA |
| | | | | | | GTTCTACAAGCAGCTG |
| | | | | | | TACTGGCTGCTGAGAC |
| | | | | | | TGACCCTGAGCCTGAG |
| | | | | | | ACACAGCGTGACAGGC |
| | | | | | | ACCGACGAGGATTTCA |
| | | | | | | TCCTGTCTCCAGTGGC |
| | | | | | | CGACGAGAATGGCGT |
| | | | | | | GTTCTTTGACTCTAGG |
| | | | | | | AAGGCCACCGACAAGC |
| | | | | | | AGCCTAAGGACGCTGA |
| | | | | | | TGCTAACGGCGCCTAC |
| | | | | | | CATATCGCCCTGAAAG |
| | | | | | | GCCTGTGGAATCTCCA |
| | | | | | | GCAGATCAGACAGCAC |
| | | | | | | GACTGGAACGTGGAAA |
| | | | | | | AGCCCAAAAAGCTGAA |
| | | | | | | CCTCGCCATGAAGAAC |
| | | | | | | GAAGAGTGGTTCGGCT |
| | | | | | | TCGCTCAGAAGAAGAA |
| | | | | | | GTTTAGAGCCAGCGGC |
| | | | | | | GGCAAGAGGCCTGCC |
| | | | | | | GCTACAAAAAAAGCCG |
| | | | | | | GCCAGGCCAAGAAAAA |
| | | | | | | GAAGTGACCACACCCC |
| | | | | | | CATTCCCCCACTCCAG |
| | | | | | | ATAGAACTTCAGTTAT |
| | | | | | | ATCTCACGTGTCTGGA |
| | | | | | | GTT |
| MG29-1 | — | 5831 | hH29-4_37 | Nucleotide | N.A. | mC*mU*mU*U*UAAUU |
| sgRNA | | | | | | mUmCmUmACU*G*U*U |
| targeting | | | | | | *GUAGAUCCCCAGAfCf |
| human | | | | | | CfUfGfUfAfAfUfAfG*fU*f |
| HAO-1 | | | | | | CfA*fU*mA |
| MG29-1 | — | 5832 | hH29-21-37 | Nucleotide | N.A. | mC*mU*mU*U*UAAUU |
| sgRNA | | | | | | mUmCmUmACU*G*U*U |
| targeting | | | | | | *GUAGAUGGACAGAfGf |
| human | | | | | | GfGfUfCfAfGfCfAfU*fG*f |
| HAO-1 | | | | | | CfC*fA*mA |
| MG29-1 | — | 5833 | hH29-23-37 | Nucleotide | N.A. | mC*mU*mU*U*UAAUU |
| sgRNA | | | | | | mUmCmUmACU*G*U*U |
| targeting | | | | | | *GUAGAUUCAGCCUfGf |
| human | | | | | | UfCfAfGfUfCfCfCfU*fG*f |
| HAO-1 | | | | | | GfG*fA*mA |
| MG29-1 | — | 5834 | hH29-41-37 | Nucleotide | N.A. | mC*mU*mU*U*UAAUU |
| sgRNA | | | | | | mUmCmUmACU*G*U*U |
| targeting | | | | | | *GUAGAUAUAUCUUfCf |
| human | | | | | | CfCfAfGfCfUfGfAfU*fA*f |
| HAO-1 | | | | | | GfA*fU*mG |
| Guide | — | 5835 | Chemistry 37 | Nucleotide | N.A. | mC*mU*mU*U*UAAUU |
| modification | | | (22 nt spacer) | | | mUmCmUmACU*G*U*U |
| chemistry | | | | | | *GUAGAUNNNNNNNfNf |
| | | | | | | NfNfNfNfNfNfNfNfN*fN*f |
| | | | | | | NfN*fN*mN |
| MG29-1 | — | 5836 | mH29-29-37 | Nucleotide | N.A. | mC*mU*mU*U*UAAUU |
| sgRNA | | | | | | mUmCmUmACU*G*U*U |
| targeting | | | | | | *GUAGAUCCUUAGGfAf |
| mouse | | | | | | GfAfAfAfAfUfGfCfC*fA*f |
| HAO-1 | | | | | | AfA*fU*mC |
| MG29-1 | — | 5837 | mH29-29-44 | Nucleotide | N.A. | mC*mU*mU*U*U*AAUU |
| sgRNA | | | | | | mUmCmUmACU*G*U*U |
| targeting | | | | | | *GUAGAUCCUUAGGfAf |
| mouse | | | | | | GfAfAfAfAfUfG*fCfC*fA |
| HAO-1 | | | | | | *fA*fA*fU*mC |
| MG29-1 | — | 5838 | mH29-29s-37 | Nucleotide | N.A. | mC*mU*mU*U*UAAUU |
| sgRNA | | | | | | mUmCmUmACU*G*U*U |
| targeting | | | | | | *GUAGAUCCUUAGGfAf |
| mouse | | | | | | GfAfAfAfAfUfG*fCfC*fA |
| HAO-1 | | | | | | *fA*mA |
| MG29-1 | — | 5839 | mH29-29s-44 | Nucleotide | N.A. | mC*mU*mU*U*UAAUU |
| sgRNA | | | | | | mUmCmUmACU*G*U*U |
| targeting | | | | | | *GUAGAUCCUUAGGfAf |
| mouse | | | | | | GfAfAfAfAfUfG*fC*fC*f |
| HAO-1 | | | | | | A*fA*mA |
| MG29-1 | — | 5840 | mH29-29-50 | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HAO-1 | | | | | | *G*U*U*GUAGAUCCUU |
| | | | | | | AGGfAfGfAfAfAfAfUfGf |
| | | | | | | CfC*fA*fAfA*fU*mC |
| MG29-1 | — | 5841 | mH29-29-50b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HAO-1 | | | | | | *G*U*U*GUAGAUCCUU |
| | | | | | | AGGfAfGfAfAfAfAfUfGf |
| | | | | | | CfC*fA*fA*mA |
| MG29-1 | — | 5842 | mH29-29-51b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HAO-1 | | | | | | *G*U*U*GUAGAUCCUU |
| | | | | | | AGGAGAAAAUGCC*A* |
| | | | | | | mA*mA |
| MG29-1 | — | 5843 | mH29-29-52b | Nucleotide | N.A. | mG*mU*mU*GAGAAUC |
| sgRNA | | | | | | *mG*mA*mA*mAGAUU |
| targeting | | | | | | CUCAAC*mC*mU*mU*U |
| mouse | | | | | | *UAAUUmUmCmUmACU |
| HAO-1 | | | | | | *G*U*U*GUAGAUCCUU |
| | | | | | | AGGAfGAfAAfAUfG*C |
| | | | | | | *fCA*fA*mA |
| MG29-1 | — | 5844 | mH29-29-53b | Nucleotide | N.A. | mG*mU*mU*mG*mA*m |
| sgRNA | | | | | | G*mA*mA*mU*mC*mG* |
| targeting | | | | | | mA*mA*mA*mG*mA*m |
| mouse | | | | | | mA*mC*mC*mU*mU*U* |
| HAO-1 | | | | | | UAAUUmUmCmUmACU* |
| | | | | | | G*U*U*GUAGAUCCUUA |
| | | | | | | GGfAfGfAfAfAfAfUfG |
| | | | | | | fCfC*fA*fA*mA |
| MG29-1 | — | 5845 | mH29-29-54b | Nucleotide | N.A. | mG*mU*mU*mG*mA*m |
| sgRNA | | | | | | G*mA*mA*mU*mC*mG* |
| targeting | | | | | | mA*mA*mA*mG*mA*m |
| mouse | | | | | | U*mU*mC*mU*mC*mA* |
| HAO-1 | | | | | | mA*mC*mC*mU*mU*U* |
| | | | | | | UAAUUmUmCmUmACU* |
| | | | | | | G*U*U*GUAGAUCCUUA |
| | | | | | | GGAGAAAAUGCC*A*m |
| | | | | | | A*mA |
| DNA | — | 5846 | DNA sequence | Nucleotide | N.A. | AAAAGCCAGCTCCAGC |
| sequence | | | encoding the | | | AGGCGCTGCTCACTCC |
| encoding | | | MG29-1 | | | TCCCCATCCTCTCCCT |
| the | | | messenger RNA | | | CTGTCCCTCTGTCCCT |
| MG29-1 | | | | | | CTGACCCTGCACTGTC |
| messenger | | | | | | CCAGCACCATGGCCCC |
| RNA | | | | | | TAAGAAGAAGAGAAAA |
| | | | | | | GTCGGCGGAGGCGGC |
| | | | | | | AGCTTCAACAACTTCA |
| | | | | | | TCAAGAAGTACAGCCT |
| | | | | | | GCAGAAAACCCTGCGC |
| | | | | | | TTCGAGCTGAAGCCTG |
| | | | | | | TGGGCGAGACAGCCG |
| | | | | | | ACTACATCGAGGACTT |
| | | | | | | CAAGAGCGAGTACCTG |
| | | | | | | AAGGACACCGTGCTGA |
| | | | | | | AGGACGAGCAGAGAG |
| | | | | | | CCAAGGACTACCAAGA |
| | | | | | | GATCAAGACCCTGATC |
| | | | | | | GACGATTACCACCGCG |
| | | | | | | AGTACATCGAAGAGTG |
| | | | | | | CCTGAGAGAACCCGTG |
| | | | | | | GACAAGAAAACCGGC |
| | | | | | | GAGATCCTGGACTTCA |
| | | | | | | CCCAGGACCTGGAAGA |
| | | | | | | TGCCTTCAGCTACTAC |
| | | | | | | CAGAAGCTGAAAGAGA |
| | | | | | | ACCCCACCGAGAACAG |
| | | | | | | AGTCGGCTGGGAGAA |
| | | | | | | AGAGCAAGAGAGCCT |
| | | | | | | GAGGAAGAAGCTGGT |
| | | | | | | CACCTCCTTCGTGGGC |
| | | | | | | AACGACGGCCTGTTCA |
| | | | | | | AGAAAGAGTTCATCAC |
| | | | | | | CAGGGACCTGCCTGAG |
| | | | | | | TGGCTGCAGAAGAAAG |
| | | | | | | GACTCTGGGGCGAGTA |
| | | | | | | CAAGGACACAGTGGAA |
| | | | | | | AACTTCAAGAAGTTCA |
| | | | | | | CCACCTACTTCAGCGG |
| | | | | | | CTTCCACGAGAACCGG |
| | | | | | | AAGAACATGTACACCG |
| | | | | | | CCGAGGCTCAGAGCAC |
| | | | | | | CGCTATCGCCAACAGA |
| | | | | | | CTGATGAACGACAACC |
| | | | | | | TGCCTAAGTTCTTTAA |
| | | | | | | CAACTACCTGGCCTAC |
| | | | | | | CAGACCATCAAAGAGA |
| | | | | | | AGCACCCCGACCTGGT |
| | | | | | | GTTCAGACTGGATGAT |
| | | | | | | GCTCTGCTGCAGGCCG |
| | | | | | | CTGGCGTGGAACATCT |
| | | | | | | GGATGAGGCTTTCCAG |
| | | | | | | CCTAGATACTTCAGCA |
| | | | | | | GACTGTTCGCCCAGAG |
| | | | | | | CGGCATCACCGCTTTC |
| | | | | | | AACGAGCTGATCGGCG |
| | | | | | | GCAGAACCACAGAGAA |
| | | | | | | CGGCGAGAAGATCCA |
| | | | | | | GGGCCTGAACGAGCA |
| | | | | | | GATCAACCTGTACAGA |
| | | | | | | CAGCAGAACCCCGAGA |
| | | | | | | AGGCCAAGGGCTTCCC |
| | | | | | | CAGATTCATGCCTCTG |
| | | | | | | TTCAAGCAGATCCTGA |
| | | | | | | GCGACAGAGAGACAC |
| | | | | | | ACAGCTTTCTGCCCGA |
| | | | | | | CGCCTTCGAGAACGAC |
| | | | | | | AAAGAGCTGCTCCAGG |
| | | | | | | CTCTGAGAGACTACGT |
| | | | | | | GGACGCCGCCACATCT |
| | | | | | | GAGGAAGGCATGATCA |
| | | | | | | GCCAGCTGAACAAGGC |
| | | | | | | CATGAACCAGTTCGTG |
| | | | | | | ACCGCCGACCTGAAGA |
| | | | | | | GAGTGTACATCAAGAG |
| | | | | | | CGCCGCTCTGACCAGC |
| | | | | | | CTGAGCCAAGAGCTGT |
| | | | | | | TCCACTTCTTCGGCGT |
| | | | | | | GATCAGCGACGCTATC |
| | | | | | | GCTTGGTACGCCGAGA |
| | | | | | | AGAGACTGAGCCCCAA |
| | | | | | | GAAGGCCCAAGAGTCT |
| | | | | | | TTCCTGAAGCAAGAGG |
| | | | | | | TGTACGCCATCGAGGA |
| | | | | | | ACTGAACCAGGCTGTC |
| | | | | | | GTGGGCTACATCGACC |
| | | | | | | AGCTGGAAGATCAGAG |
| | | | | | | CGAGCTGCAGCAACTG |
| | | | | | | CTGGTGGACCTGCCAG |
| | | | | | | ATCCTCAGAAACCCGT |
| | | | | | | GTCCAGCTTCATCCTG |
| | | | | | | ACACACTGGCAGAAGT |
| | | | | | | CTCAAGAGCCCCTGCA |
| | | | | | | GGCAGTGATCGCCAAG |
| | | | | | | GTGGAACCTCTGTTCG |
| | | | | | | AACTGGAAGAACTGAG |
| | | | | | | CAAGAACAAGAGGGC |
| | | | | | | CCCAAAGCACGACAAG |
| | | | | | | GACCAAGGCGGCGAG |
| | | | | | | GGATTTCAGCAGGTCG |
| | | | | | | ACGCCATCAAGAACAT |
| | | | | | | GCTGGACGCCTTCATG |
| | | | | | | GAAGTGTCCCACGCTA |
| | | | | | | TCAAGCCCCTGTACCT |
| | | | | | | GGTCAAGGGAAGAAA |
| | | | | | | GGCCATCGACATGCCC |
| | | | | | | GACGTGGACACCGGCT |
| | | | | | | TCTACGCTGATTTCGC |
| | | | | | | CGAGGCCTACAGCGCC |
| | | | | | | TACGAGCAAGTGACAG |
| | | | | | | TGTCCCTGTACAACAA |
| | | | | | | GACCAGAAACCACCTG |
| | | | | | | TCCAAGAAGCCCTTCA |
| | | | | | | GCAAGGACAAGATCAA |
| | | | | | | GATCAACTTCGACGCC |
| | | | | | | CCTACACTGCTGAACG |
| | | | | | | GCTGGGACCTGAACAA |
| | | | | | | AGAGAGCGACAACAA |
| | | | | | | GTCCATCATCCTGCGG |
| | | | | | | AAGGACGGCAACTTCT |
| | | | | | | ACCTGGCAATCATGCA |
| | | | | | | CCCCAAGCACACCAAG |
| | | | | | | GTGTTCGACTGCTACT |
| | | | | | | CTGCCTCTGAGGCTGC |
| | | | | | | CGGCAAGTGCTACGAG |
| | | | | | | AAGATGAACTACAAGC |
| | | | | | | TGCTGAGCGGCGCCAA |
| | | | | | | CAAGATGCTGCCTAAG |
| | | | | | | GTGTTCTTTAGCAAGA |
| | | | | | | AGGGCATCGAGACATT |
| | | | | | | CAGCCCTCCACAAGAA |
| | | | | | | ATCCTGGACCTGTACA |
| | | | | | | AGAACAACGAGCATAA |
| | | | | | | GAAGGGCGCCACCTTC |
| | | | | | | AAGCTGGAATCCTGCC |
| | | | | | | ACAAGCTGATCGATTT |
| | | | | | | CTTCAAGCGGAACATC |
| | | | | | | CCCAAGTACAAGGTGC |
| | | | | | | ACCCTACCGACAACTT |
| | | | | | | TGGCTGGGACGTGTTC |
| | | | | | | GGCTTTCACTTCAGCC |
| | | | | | | CTACCAGCAGCTACGG |
| | | | | | | CGACCTGTCTGGCTTC |
| | | | | | | TACAGAGAGGTGGAA |
| | | | | | | GCCCAGGGATACAAGC |
| | | | | | | TGTGGTTCAGCGACGT |
| | | | | | | GTCCGAGGCTTACATC |
| | | | | | | AACAAATGCGTGGAAG |
| | | | | | | AGGGCAAGCTGTTCCT |
| | | | | | | GTTCCAAATCTACAAC |
| | | | | | | AAGGACTTCTCCCCTA |
| | | | | | | ACTCCACCGGCAAGCC |
| | | | | | | CAACCTGCACACCCTG |
| | | | | | | TATTGGAAGGGCCTGT |
| | | | | | | TCGAGCCCGAGAACCT |
| | | | | | | GAAAGACGTGGTGCTG |
| | | | | | | AAGCTGAATGGCGAG |
| | | | | | | GCCGAGATCTTCTACC |
| | | | | | | GGAAGCACAGCATCAA |
| | | | | | | GCACGAGGACAAGAC |
| | | | | | | CATCCACAGAGCTAAG |
| | | | | | | GACCCTATCGCTAACA |
| | | | | | | AGAACGCTGACAACCC |
| | | | | | | CAAGAAACAGAGCGTG |
| | | | | | | TTCGATTACGACATCA |
| | | | | | | TCAAGGATAAGCGGTA |
| | | | | | | TACCCAGGACAAGTTC |
| | | | | | | TTCTTCCACGTGCCAA |
| | | | | | | TCAGCCTGAACTTCAA |
| | | | | | | AAGCCAGGGCGTCGT |
| | | | | | | GCGGTTCAACGATAAG |
| | | | | | | ATCAACGGCCTGCTGG |
| | | | | | | CCGCTCAGGACGATGT |
| | | | | | | GCATGTGATCGGCATC |
| | | | | | | GACAGAGGCGAGAGA |
| | | | | | | CATCTGCTGTACTACA |
| | | | | | | CCGTGGTCAACGGCAA |
| | | | | | | GGGCGAAGTGGTGGA |
| | | | | | | ACAGGGCAGCCTGAAT |
| | | | | | | CAGGTGGCCACAGATC |
| | | | | | | AGGGCTACGTGGTGG |
| | | | | | | ATTACCAGCAGAAGCT |
| | | | | | | GCACGCCAAAGAGAAA |
| | | | | | | GAACGCGACCAGGCC |
| | | | | | | AGAAAGAACTGGTCCA |
| | | | | | | CCATCGAGAACATCAA |
| | | | | | | AGAACTGAAGGCCGG |
| | | | | | | CTACCTGAGCCAGGTG |
| | | | | | | GTGCATAAGCTGGCTC |
| | | | | | | AGCTGATCGTGAAGCA |
| | | | | | | CAACGCCATCGTGTGC |
| | | | | | | CTCGAGGACCTGAATT |
| | | | | | | TCGGCTTCAAGAGGGG |
| | | | | | | CAGATTCAAGGTCGAG |
| | | | | | | AAACAGGTGTACCAGA |
| | | | | | | AGTTCGAGAAGGCTCT |
| | | | | | | GATCGACAAGCTGAAC |
| | | | | | | TACCTCGTGTTCAAAG |
| | | | | | | AGAGAGGCGCCACAC |
| | | | | | | AGGCTGGCGGATACCT |
| | | | | | | GAATGCTTACCAGCTG |
| | | | | | | GCCGCACCTTTCGAGA |
| | | | | | | GCTTTGAGAAGCTGGG |
| | | | | | | CAAGCAGACCGGCATC |
| | | | | | | CTGTACTACGTGCGGA |
| | | | | | | GCGACTACACCAGCAA |
| | | | | | | GATCGACCCTGCTACC |
| | | | | | | GGCTTCGTGGACTTTC |
| | | | | | | TGAAGCCTAAGTACGA |
| | | | | | | GAGCATGGCCAAGAG |
| | | | | | | CAAAGTGTTCTTCGAG |
| | | | | | | TCCTTCGAGCGCATCC |
| | | | | | | AGTGGAACCAGGCCAA |
| | | | | | | AGGCTACTTCGAGTTC |
| | | | | | | GAGTTTGACTACAAGA |
| | | | | | | AGATGTGCCCCAGCAG |
| | | | | | | AAAGTTCGGCGACTAC |
| | | | | | | AGAACCAGATGGGTCG |
| | | | | | | TGTGCACCTTCGGCGA |
| | | | | | | CACCCGCTACCAGAAC |
| | | | | | | AGAAGAAACAAGAGCA |
| | | | | | | GCGGCCAGTGGGAGA |
| | | | | | | CAGAGACAATCGATGT |
| | | | | | | GACAGCCCAGCTGAAA |
| | | | | | | GCCCTGTTCGCCGCTT |
| | | | | | | ACGGCATCACATACAA |
| | | | | | | TCAAGAGGATAACATC |
| | | | | | | AAGGACGCCATTGCCG |
| | | | | | | CCGTGAAGTACACCAA |
| | | | | | | GTTCTACAAGCAGCTG |
| | | | | | | TACTGGCTGCTGAGAC |
| | | | | | | TGACCCTGAGCCTGAG |
| | | | | | | ACACAGCGTGACAGGC |
| | | | | | | ACCGACGAGGATTTCA |
| | | | | | | TCCTGTCTCCAGTGGC |
| | | | | | | CGACGAGAATGGCGT |
| | | | | | | GTTCTTTGACTCTAGG |
| | | | | | | AAGGCCACCGACAAGC |
| | | | | | | AGCCTAAGGACGCTGA |
| | | | | | | TGCTAACGGCGCCTAC |
| | | | | | | CATATCGCCCTGAAAG |
| | | | | | | GCCTGTGGAATCTCCA |
| | | | | | | GCAGATCAGACAGCAC |
| | | | | | | GACTGGAACGTGGAAA |
| | | | | | | AGCCCAAAAAGCTGAA |
| | | | | | | CCTCGCCATGAAGAAC |
| | | | | | | GAAGAGTGGTTCGGCT |
| | | | | | | TCGCTCAGAAGAAGAA |
| | | | | | | GTTTAGAGCCAGCGGC |
| | | | | | | GGCAAGAGGCCTGCC |
| | | | | | | GCTACAAAAAAAGCCG |
| | | | | | | GCCAGGCCAAGAAAAA |
| | | | | | | GAAGTGACCACACCCC |
| | | | | | | CATTCCCCCACTCCAG |
| | | | | | | ATAGAACTTCAGTTAT |
| | | | | | | ATCTCACGTGTCTGGA |
| | | | | | | GTT |
| MG55 | — | 6031 | MG55 sgRNA | Nucleotide | Unknown | AAATATTTCATTAAGT |
| active | | | | | | ACCGAATTTAAAAAAT |
| effector | | | | | | AGGATTGCAGAAAGTG |
| sgRNA | | | | | | CAATTAGGCTGGTTGT |
| | | | | | | GCAGCCTTAATCTGAG |
| | | | | | | GGATTAATCCACTCGG |
| | | | | | | AAAGTACCTTTATTGA |
| | | | | | | AAAATGAAAGGTATTC |
| | | | | | | ACAAC |
| MG55 | A6032 | — | MG55 PAM (5′) | Nucleotide | Unknown | YTn |
| active | | | | | | |
| effector | | | | | | |
| PAM | | | | | | |
| (5′) | | | | | | |
| MG91 | — | 6033 | MG91-15 sgRNA1 | Nucleotide | Unknown | UAGAGAAAAUUAUAUA |
| active | | | | | | UUAGGUUUUGUUAAGC |
| effector | | | | | | CUAACAAUCGUUAAGU |
| sgRNA | | | | | | GUUCUUUGGAAUAUUG |
| | | | | | | AUUGUAAAUCUAUUUU |
| | | | | | | GGGAAAUGAAAAGGC |
| | | | | | | AAAAAUUACAGUUAUC |
| | | | | | | AAUUAAUUGAGAAGAG |
| | | | | | | UAUAGAGUCAGUUUUA |
| | | | | | | UAGUACCAAAAUAUAC |
| | | | | | | CUUAAUUCUUUAAGAA |
| | | | | | | AUUAAAUUUAAGGUAA |
| | | | | | | UAACAAG |
| MG91 | — | 6034 | MG91-32 sgRNA1 | Nucleotide | Unknown | UGCACAUCGGGUAUG |
| active | | | | | | UGUGGGGUCGAGUAA |
| effector | | | | | | GGCCGACGUUGUCCG |
| sgRNA | | | | | | CUACAACUUAGCCGUC |
| | | | | | | GGGCGGUUGGGCAAC |
| | | | | | | CGAUCGGAAGCGGAA |
| | | | | | | CCUGGAAUAAGGCCA |
| | | | | | | GGCAGCGGCACUGCC |
| | | | | | | GUCAAGCGGGAAUGA |
| | | | | | | AGUCCAGUAGUACGUA |
| | | | | | | ACCAGUAACUUACAAA |
| | | | | | | GAAAUUUGUAAGGUAC |
| | | | | | | UUACAGG |
| MG91 | — | 6035 | MG91-87 sgRNA1 | Nucleotide | Unknown | GAAUUGAUGCUUCGU |
| active | | | | | | GCAUCUAAAAAUAUAC |
| effector | | | | | | UGGGAAUUGUAUUCCC |
| sgRNA | | | | | | GAAGUGAGCGUUAAU |
| | | | | | | UGGCACAGUGGUGUC |
| | | | | | | AUUGCUCAUCAAAAGA |
| | | | | | | AGAAUUGGAAAAACAG |
| | | | | | | CGAACUUCAUCUCGUU |
| | | | | | | UCUUCACCUUUGGUGC |
| | | | | | | AAGCAAAGGUAAUGAA |
| | | | | | | GUGAAGGCUUUUUAG |
| | | | | | | UACAAUCUCAUACCCC |
| | | | | | | UAACUGUGUGAUACUA |
| | | | | | | UGCCCUCGAAAGAGG |
| | | | | | | GGUAAAUACAGG |
| MG91 | — | 6036 | MG91-87 sgRNA2 | Nucleotide | Unknown | UGGGAAUUGUAUUCCC |
| active | | | | | | GAAGUGAGCGUUAAU |
| effector | | | | | | UGGCACAGUGGUGUC |
| sgRNA | | | | | | AUUGCUCAUCAAAAGA |
| | | | | | | AGAAUUGGAAAAACAG |
| | | | | | | CGAACUUCAUCUCGUU |
| | | | | | | UCUUCACCUUUGGUGC |
| | | | | | | AAGCAAAGGUAAUGAA |
| | | | | | | GUGAAGGCUUUUUAG |
| | | | | | | UACAAUCUCAUACCCC |
| | | | | | | UAACUGUGUGAUACUA |
| | | | | | | UGCCCUCGAAAGAGG |
| | | | | | | GGUAAAUACAGG |
| MG91 | A6037 | — | MG91-15 PAM | Nucleotide | Unknown | TtTYn |
| active | | | (5′) | | | |
| effector | | | | | | |
| PAM | | | | | | |
| (5′) | | | | | | |
| MG91 | A6038 | — | MG91-32 PAM | Nucleotide | Unknown | GnYYn |
| active | | | (5′) | | | |
| effector | | | | | | |
| PAM | | | | | | |
| (5′) | | | | | | |
| MG91 | A6039 | — | MG91-87 PAM | Nucleotide | Unknown | wCCC |
| active | | | (5′) | | | |
| effector | | | | | | |
| PAM | | | | | | |
| (5′) | | | | | | |
| MG91 | — | 6040 | MG91-2 IG2 | Nucleotide | Unknown | AAATAACATACAGAGG |
| intergenic | | | | | | TTTTGTTAAGCCTCAC |
| region | | | | | | AATCTTAATAAATAAG |
| potentially | | | | | | TGTTCTTTGAAAATAT |
| encoding | | | | | | TTAGTTGATTGTAAAT |
| tracrRNA | | | | | | CTATTTTGGGAAATAA |
| | | | | | | AAAAACAAAAATTACA |
| | | | | | | GTTATTAGTTAACTAA |
| | | | | | | GAAGAGTATAGAGTTA |
| | | | | | | GTTTTAAAGTACCAAA |
| | | | | | | ATATACCCTAAATTAT |
| | | | | | | TGTTTTTCAAAACTTT |
| | | | | | | ATAGAATATAT |
| MG91 | — | 6041 | MG91-10 IG1 | Nucleotide | Unknown | TATGGCGTGTAATCAC |
| intergenic | | | | | | CTGGGAATGAAATAAA |
| region | | | | | | AAGGCCATGATTTGCA |
| potentially | | | | | | TACTCGGAGACCTGCT |
| encoding | | | | | | CTATACTCCCATTCCC |
| tracrRNA | | | | | | GAAGGAACTGACTGTT |
| | | | | | | ATTTTTGCATTCTACC |
| | | | | | | ATTCTGCATAATCGTT |
| | | | | | | TGTCGCACCAGCCGCC |
| | | | | | | CACTGGCATTAGCTTA |
| | | | | | | ACGTACGCAAGGCCTG |
| | | | | | | ACAACACCTTTATGTC |
| | | | | | | GCAAATATACGAATTA |
| | | | | | | TTATTGAAAGTGCGTA |
| | | | | | | CAGAATTCAGAATGAA |
| | | | | | | AGTGCTATACTTTAAC |
| | | | | | | AGTATTTATCCAAAAC |
| | | | | | | GTTTAATATCTTACAC |
| | | | | | | ATCAATTCATGTTTGT |
| | | | | | | AAATCACATTTCATTT |
| | | | | | | ATTAATCAGACCTGTT |
| | | | | | | TAACGTAGTAACAAGA |
| | | | | | | ACGAAATTACTGACTT |
| | | | | | | TCAATCTGCCTTCTAT |
| | | | | | | ATGATAGCAGAAAAAT |
| | | | | | | TCTCTAGAGAATTTCT |
| | | | | | | TCCTGTATAACTAAGT |
| | | | | | | AGCAAATGCTATATCA |
| | | | | | | CTTAGTAAGGACAAAA |
| | | | | | | TTCTCTAGAGAATTTC |
| | | | | | | TTTACTACTCTACAGC |
| | | | | | | TAAGAAATGAGGAACA |
| | | | | | | ACAGACTGGGTTTTAC |
| | | | | | | TTTATAACCGATAGAG |
| | | | | | | ATGGAACGGGTGAGT |
| | | | | | | GTCGGAAGATGTTTAA |
| | | | | | | ATCAAGATTATCGAGA |
| | | | | | | AATAATTGGCATTCTC |
| | | | | | | AGGAGTATTTACTAAA |
| | | | | | | TTTGCACGAAAATACG |
| | | | | | | AAAAGCATT |
| MG91 | — | 6042 | MG91-52 IG1 | Nucleotide | Unknown | GATATAAAGAGGCATT |
| intergenic | | | | | | GTCAGGCCTCGCGGAC |
| region | | | | | | GTTAAGCTAATGCCGG |
| potentially | | | | | | TGGGCGGCTGGTCCG |
| encoding | | | | | | ACGAATTGGTGAGTGG |
| tracrRNA | | | | | | AAGAGGAATGCGAAAA |
| | | | | | | TGACAGTCACCCTCGT |
| | | | | | | AAGAGGGCTGAGTTAT |
| | | | | | | AGAGTATATCCGCGAG |
| | | | | | | TACGCAAGCCGTGACT |
| | | | | | | ACATACTGGCATTGAG |
| | | | | | | CCAGTTAGACATTATG |
| | | | | | | A |
| MG91 | — | 6043 | MG91-61 IG2 | Nucleotide | Unknown | TAATTAAGGTTGTGAT |
| intergenic | | | | | | CTTTATGATTGAGATT |
| region | | | | | | GCAACCTTTTTTTAAA |
| potentially | | | | | | AAAATATGCAACAAAA |
| encoding | | | | | | CGCCCCAAAACGCATC |
| tracrRNA | | | | | | ATTTAGGGGTGTTAGG |
| | | | | | | GTCTTGAATTTGTTAA |
| | | | | | | ATCAAGACTTTTATTT |
| | | | | | | TGAATATTTTGGCTCC |
| | | | | | | ACAAGGGAATTTTCGC |
| | | | | | | AATTTTGCACTGCACA |
| | | | | | | CCAGCAATGGCGTGTG |
| | | | | | | GGGGCTTGTAGGCCC |
| | | | | | | GACGCATCCTCGCTTT |
| | | | | | | AAAACTCAGCCATCGG |
| | | | | | | GCGGTTATGGGGAGCT |
| | | | | | | GAACGGAAACGGAAAT |
| | | | | | | GGGAATAAGACAATGC |
| | | | | | | AGCGCAAGCAGAGCG |
| | | | | | | TGGATGAAGTCTCAGA |
| | | | | | | GTACGCGACCATTGAC |
| | | | | | | TTGCAAAAAGTGATCT |
| | | | | | | TTGACCAACTGAAGGG |
| | | | | | | TGTAATACACACCGCT |
| | | | | | | GTAAGGCAAATACAGG |
| | | | | | | CTGCAGACTGGCCATC |
| | | | | | | CCAGAATACAATAA |
| MG91 | — | 6044 | MG91-69 IG2 | Nucleotide | Unknown | TAAAAAAAGATATTAG |
| intergenic | | | | | | GTTTTGTTAAGCCTAA |
| region | | | | | | CAATCGTTAAGTGTTC |
| potentially | | | | | | TTTGGAATATTGATTG |
| | | | | | | TAAATCTATTTTGGGA |
| encoding | | | | | | AATAAAAAAGCAAAAA |
| tracrRN | | | | | | TTACAGTTATCAGTTT |
| A | | | | | | ACTGAGAAGAGTATAG |
| | | | | | | AGTTAGTTTTAAAGTA |
| | | | | | | CCAAAATATACCCTAA |
| | | | | | | ATTATTGTTTTTCAAA |
| | | | | | | ACTTTATAGAATATAT |
| MG91 | — | 6045 | MG91-94 IG1 | Nucleotide | Unknown | CCGTAGTGCATAATCA |
| intergeni | | | | | | CCTGAGAATGAATGAA |
| c region | | | | | | AAAGGCCATGATATGC |
| potential | | | | | | ATACTTGGGGACTTGC |
| ly | | | | | | TCTATACTCCCATTCC |
| encoding | | | | | | CGAAGGAATTGACTGT |
| tracrRN | | | | | | TATTTTTGCATTCCAC |
| A | | | | | | CATTCTGCAAATCGTT |
| MG91 | — | 6046 | MG91-101 IG2 | Nucleotide | Unknown | TGTCGCACCAGCCGCC |
| intergenic | | | | | | CACTGGCATTAAGCTT |
| region | | | | | | AACGTGCGCAAGGCCT |
| potentially | | | | | | GACAACACCTTTATGG |
| encoding | | | | | | GTGCAAAGATACTAAA |
| tracrRN | | | | | | AGTTTTCAAAAGAAGA |
| A | | | | | | TAGGGAAAAGAGAAAA |
| | | | | | | AACTTCAAATCCATTT |
| | | | | | | TGGCGTTGCAGCGTTG |
| | | | | | | CAGTGTTGCAGTTCCA |
| | | | | | | AAATAGATTCTGCGAT |
| | | | | | | AGTCAAAAAATAACTC |
| | | | | | | TATATTTAATAAATAT |
| | | | | | | AGAAGTATT |
| | | | | | | TTCAGGAATTCAAAGA |
| | | | | | | TCACCTTTTTTGCAAG |
| | | | | | | TTACTGGTCACGTACT |
| | | | | | | ACTGGACTTCATTCCT |
| | | | | | | GCTTGACGGCAGTGCC |
| | | | | | | GCTGCATGGCCTTATT |
| | | | | | | CCAGGTTCCGCTTCCG |
| | | | | | | ATTGGCTGCCCAACCG |
| | | | | | | CCCGACGGCTAAGTTG |
| | | | | | | TAGCGGACAGCGTTGG |
| | | | | | | CCCTACAAGACCCCAC |
| | | | | | | ACATTCCCGATGTGCA |
| | | | | | | TTGCAAAGTTCGGGAA |
| | | | | | | TGATATCGAGAAAGGC |
| | | | | | | AAAATCTGGCAAAACT |
| | | | | | | TTGTCTTGATTTAACA |
| | | | | | | CATTTATGATTTTTCC |
| | | | | | | GGCGAAAAAATGGTCG |
| | | | | | | CGAAAAATGTCATCCA |
| | | | | | | GGATATATGAAGACTC |
| | | | | | | CAGCGGAAAAAGGTTT |
| | | | | | | AACGCCGTTGTAAACT |
| | | | | | | TAGTTTTCTTTGGAGT |
| | | | | | | AAACAGGGTAACCTCT |
| | | | | | | TGTGGAAATTCGGACA |
| | | | | | | GGATGAGTGTTGATTG |
| | | | | | | CTTTATCCGGATGTTA |
| | | | | | | TTTTGCTATAGTATTT |
| MG91 | — | 6047 | MG91-107 IG2 | Nucleotide | Unknown | GTATAAACACTATTGG |
| intergenic | | | | | | ACTGTGACAAATAAAG |
| region | | | | | | TTAGGATAGGATGTTC |
| potentially | | | | | | TTACTTTAAATAGTAT |
| | | | | | | TTGCTATCGTTCAAAA |
| encoding | | | | | | AGGCAAGATAGCCATT |
| tracrRNA | | | | | | TTTACATGATAACATT |
| | | | | | | CTGCTTTATTTCATCA |
| | | | | | | CATCTTTGTTTTAGCA |
| | | | | | | ATGGCAATTGTTTGCC |
| | | | | | | ATTAACGCTAAGATTG |
| | | | | | | GATTACAAATCCATAT |
| | | | | | | TTCT |
| MG91 | — | 6048 | MG91-155 IG1 | Nucleotide | Unknown | CCTATGGTTGGTAATC |
| intergenic | | | | | | ACCTAGGAATGAATAA |
| region | | | | | | AAAGACTATGATAAGC |
| potentially | | | | | | ATACTCGGGGACTTGC |
| encoding | | | | | | TCTATACTCCCGTTCT |
| tracrRNA | | | | | | CGAAGGAACTGACTGT |
| | | | | | | TATTTTTGCATTCCAC |
| | | | | | | CATTCTGCAAATCGTT |
| | | | | | | TGTCGGACCAGCCGCC |
| | | | | | | CACTGGCATTAGCTTG |
| | | | | | | ACGTCCGCAAGGCCTA |
| | | | | | | ACAACGCCTTTCTTTT |
| | | | | | | GCAAATATACGGATTT |
| | | | | | | ATATTTCTTTCACAAA |
| | | | | | | ATTAATGAGGAACTTT |
| | | | | | | TTCATATTCCTTTTGTC |
| | | | | | | CATCTGTTACTGCCAC |
| | | | | | | ATGATTTTTCGTAGGA |
| | | | | | | AAGACCTTGGAGGCG |
| | | | | | | GATGGATTGAAA |
| MG91 | — | 6049 | MG91-201 IG2 | Nucleotide | Unknown | ATATATTCTATAAAGT |
| intergenic | | | | | | TTTGAAAAACAATAAT |
| region | | | | | | TTAGGGTATATTTTGG |
| potentially | | | | | | TACTTTAAAACTAACT |
| encoding | | | | | | CTATACTCTTCTTAGT |
| tracrRNA | | | | | | TAACTAATAACTGTAA |
| | | | | | | TTTTTGTTTTTTTATTT |
| | | | | | | CCCAAAATAGATTTAC |
| | | | | | | AATCAACTAAATATTT |
| | | | | | | CAAAGAACACTTAATT |
| | | | | | | ATTAAGATTGTGAGGC |
| | | | | | | TTAACAAAACCTCTGT |
| | | | | | | ATGTTATTTTTT |
| MG91 | — | 6050 | MG91-2 Repeat | Nucleotide | Unknown | GGTAGATATACCTATT |
| CRISPR | | | | | | AAAGTTAGGGTACTAA |
| repeat | | | | | | CAAG |
| MG91 | — | 6051 | MG91-10 Repeat | Nucleotide | Unknown | GGTGTTAATCACCCGG |
| CRISPR | | | | | | AAATGTAGGCTATTGA |
| repeat | | | | | | CAGG |
| MG91 | — | 6052 | MG91-52 Repeat | Nucleotide | Unknown | GGTGTAAGACACCTGG |
| CRISPR | | | | | | AAATGTAAGGCATTGA |
| repeat | | | | | | CAGG |
| MG91 | — | 6053 | MG91-61 Repeat | Nucleotide | Unknown | CCTGTATTTGGCTTAC |
| CRISPR | | | | | | AAATTAGGATGATTAA |
| repeat | | | | | | CACC |
| MG91 | — | 6054 | MG91-69 Repeat | Nucleotide | Unknown | GGTGTATATACCTAAT |
| CRISPR | | | | | | AAAGTTAGGGTACTAA |
| repeat | | | | | | CAAG |
| MG91 | — | 6055 | MG91-94 Repeat | Nucleotide | Unknown | GGTGTTAATCACCCGA |
| CRISPR | | | | | | AAATGTAGGTCATTGA |
| repeat | | | | | | CAGGA |
| MG91 | — | 6056 | MG91-101 Repeat | Nucleotide | Unknown | GGGTGTTACACACCCG |
| CRISPR | | | | | | AATTTGCAAGGTACTT |
| repeat | | | | | | ACAGG |
| MG91 | — | 6057 | MG91-107 Repeat | Nucleotide | Unknown | CCTGTATGTGACTTTG |
| CRISPR | | | | | | AAATATAGGGTAGTTA |
| repeat | | | | | | CAAG |
| MG91 | — | 6058 | MG91-155 Repeat | Nucleotide | Unknown | GGTGTTAATCACCCAG |
| CRISPR | | | | | | AAATGTAGACTATTAA |
| repeat | | | | | | CAGG |
| MG91 | — | 6059 | MG91-201 Repeat | Nucleotide | Unknown | GCTGTATATGCCTAAT |
| CRISPR | | | | | | AAAGTTAGGGTACTAA |
| repeat | | | | | | CAAGAA |

mN: 2′-O methyl modified base N; fN: 2′-Fluoro modified base N; *: phosphorothioate linkage; N: standard ribonucleotide base

TABLE 47
Listing of PAM sequences
referred to herein

| Sequence Number | Sequence |
|---|---|
| A3863 | TA |
| A3864 | TA |
| A3865 | TR |
| A3866 | TTR |
| A3867 | TTA |
| A3872 | YYYN |
| A3873 | TTTN |
| A3874 | TTTR |
| A3876 | YTTM |
| A3879 | YN |
| A3880 | YYNW |
| A3881 | YYN |
| A3882 | YTTV |
| A3883 | TTN |
| A3884 | TTN |
| A3885 | YYN |
| A3886 | TTR |
| A3887 | TTR |
| A3888 | TTTN |
| A3889 | TTN |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Embodiments

The following embodiments are not intended to be limiting in any way.
1. An engineered nuclease system comprising:
   (a) an endonuclease comprising a RuvC domain, wherein said endonuclease is derived from an uncultivated microorganism, and wherein said endonuclease is a Cas12a endonuclease; and
   (b) an engineered guide RNA, wherein said engineered guide RNA is configured to form a complex with said endonuclease and said engineered guide RNA comprises a spacer sequence configured to hybridize to a target nucleic acid sequence.
2. An engineered nuclease system comprising:
   (a) an endonuclease having at least 75% sequence identity to any one of SEQ ID NOs: 1-3470 or a variant thereof; and
   (b) an engineered guide RNA, wherein said engineered guide RNA is configured to form a complex with said endonuclease and said engineered guide RNA comprises a spacer sequence configured to hybridize to a target nucleic acid sequence.
3. An engineered nuclease system comprising:
   (a) an endonuclease configured to bind to a protospacer adjacent motif (PAM) sequence comprising any one of SEQ ID NOs: 3890-3913 or any one of Sequence Numbers: A3863-A3889, wherein said endonuclease is a class 2, type V Cas endonuclease; and
   (b) an engineered guide RNA, wherein said engineered guide RNA is configured to form a complex with said endonuclease and said engineered guide RNA comprises a spacer sequence configured to hybridize to a target nucleic acid sequence.
4. The engineered nuclease system of any one of embodiments 1-3, wherein said endonuclease further comprises a zinc finger-like domain.
5. The engineered nuclease system of any one of embodiments 1-4, wherein said guide RNA comprises a sequence with at least 80% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, and 3851-3857.
6. An engineered nuclease system comprising:
   (a) an engineered guide RNA comprising a sequence with at least 80% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, and 3851-3857, and
   (b) a class 2, type V Cas endonuclease configured to bind to said engineered guide RNA.
7. The engineered nuclease system of any of embodiments 1-6, wherein said endonuclease is configured to bind to a protospacer adjacent motif (PAM) sequence comprising any one of Sequence Numbers: A3863-A3889, or any one of SEQ ID NOs: 3890-3913.
8. The engineered nuclease system of any one of embodiments 1-7, wherein said guide RNA comprises a sequence complementary to a eukaryotic, fungal, plant, mammalian, or human genomic polynucleotide sequence.
9. The engineered nuclease system of any one of embodiments 1-8, wherein said guide RNA is 30-250 nucleotides in length.
10. The engineered nuclease system of any one of embodiments 1-9, wherein said endonuclease comprises one or more nuclear localization sequences (NLSs) proximal to an N- or C-terminus of said endonuclease.
11. The engineered nuclease system of any one of embodiments 1-10, wherein said NLS comprises a sequence at least 80% identical to a sequence from the group consisting of SEQ ID NO: 3938-3953.
12. The engineered nuclease system of any one of embodiments 1-10, wherein said endonuclease comprises at least one of the following mutations: S168R, E172R, N577R, or Y170R when a sequence of said endonuclease is optimally aligned to SEQ ID NO: 215.
13. The engineered nuclease system of any one of embodiments 1-10, wherein said endonuclease comprises the mutations S168R and E172R when a sequence of said endonuclease is optimally aligned to SEQ ID NO: 215.
14. The engineered nuclease system of any one of embodiments 1-10, wherein said endonuclease comprises the mutations N577R or Y170R when a sequence of said endonuclease is optimally aligned to SEQ ID NO: 215.
15. The engineered nuclease system of any one of embodiments 1-10, wherein said endonuclease comprises the mutation S168R when a sequence of said endonuclease is optimally aligned to SEQ ID NO: 215.
16. The engineered nuclease system of embodiment 15, wherein said endonuclease does not comprise a mutation of E172, N577, or Y170.
17. The engineered nuclease system of any one of embodiments 1-16, further comprising a single- or double-stranded DNA repair template comprising from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence.
18. The engineered nuclease system of embodiment 17, wherein said first or second homology arm comprises a sequence of at least 40, 80, 120, 150, 200, 300, 500, or 1,000 nucleotides.
19. The engineered nuclease system of any one of embodiments 12-18, wherein said first and second homology arms are homologous to a genomic sequence of a prokaryote, bacteria, fungus, or eukaryote.
20. The engineered nuclease system of embodiments 12-19, wherein said single- or double-stranded DNA repair template comprises a transgene donor.
21. The engineered nuclease system of any one of embodiments 1-20, further comprising a DNA repair template comprising a double-stranded DNA segment flanked by one or two single-stranded DNA segments.
22. The engineered nuclease system of embodiment 21, wherein single-stranded DNA segments are conjugated to the 5′ ends of said double-stranded DNA segment.
23. The engineered nuclease system of embodiment 21, wherein said single stranded DNA segments are conjugated to the 3′ ends of said double-stranded DNA segment.
24. The engineered nuclease system of any one of embodiments 21-23, wherein said single-stranded DNA segments have a length from 4 to 10 nucleotide bases.
25. The engineered nuclease system of any one of embodiments 21-24, wherein said single-stranded DNA segments have a nucleotide sequence complementary to a sequence within said spacer sequence.
26. The engineered nuclease system of any one of embodiments 21-25, wherein said double-stranded DNA sequence comprises a barcode, an open reading frame, an enhancer, a promoter, a protein-coding sequence, a miRNA coding sequence, an RNA coding sequence, or a transgene.
27. The engineered nuclease system of any one of embodiments 21-25, wherein said double-stranded DNA sequence is flanked by a nuclease cut site.
28. The engineered nuclease system of embodiment 27, wherein said nuclease cut site comprises a spacer and a PAM sequence.
29. The engineered nuclease system of any one of embodiments 1-28, wherein said system further comprises a source of Mg²⁺.
30. The engineered nuclease system of any one of embodiments 1-29, wherein said guide RNA comprises a hairpin comprising at least 8, at least 10, or at least 12 base-paired ribonucleotides.
31. The engineered nuclease system of embodiment 30, wherein said hairpin comprises 10 base-paired ribonucleotides.
32. The engineered nuclease system of any one of embodiments 1-31, wherein:
   a) said endonuclease comprises a sequence at least 75%, 80%, or 90% identical to any one of SEQ ID NOs: 141, 215, 229, 261, or 1711-1721 or a variant thereof; and
   b) said guide RNA structure comprises a sequence at least 80%, or 90% identical to the non-degenerate nucleotides of any one of SEQ ID NOs: 3608-3609, 3853, or 3851-3857.
33. The engineered nuclease system of any one of embodiments 1-32, wherein said endonuclease is configured to bind to a PAM comprising any one of Sequence Numbers: A3863-A3889, or any one of SEQ ID NOs: 3890-3913.
34. The engineered nuclease system of any one of embodiments 1-32, wherein said endonuclease is configured to bind to a PAM comprising a sequence of YYn.
35. The engineered nuclease system of any one of embodiments 5-34, wherein said sequence identity is determined by a BLASTP, CLUSTALW, MUSCLE, MAFFT algorithm, or a CLUSTALW algorithm with the Smith-Waterman homology search algorithm parameters.
36. The engineered nuclease system of embodiment 35, wherein said sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.
37. An engineered guide RNA comprising:
   a) a DNA-targeting segment comprising a nucleotide sequence that is complementary to a target sequence in a target DNA molecule; and
   b) a protein-binding segment comprising two complementary stretches of nucleotides that hybridize to form a double-stranded RNA (dsRNA) duplex,
   wherein said two complementary stretches of nucleotides are covalently linked to one another with intervening nucleotides, and
   wherein said engineered guide ribonucleic acid polynucleotide is capable of forming a complex with an endonuclease having at least 75% sequence identity to any one of SEQ ID NOs: 1-3470, and targeting said complex to said target sequence of said target DNA molecule.
38. The engineered guide ribonucleic acid polynucleotide of embodiment 37, wherein said DNA-targeting segment is positioned 3′ of both of said two complementary stretches of nucleotides.
39. The engineered guide ribonucleic acid polynucleotide of embodiment 37-38, wherein said protein binding segment comprises a sequence having at least 70%, at least 80%, or at least 90% identity to the non-degenerate nucleotides of SEQ ID NO: 3608-3609.
40. The engineered guide ribonucleic acid polynucleotide of any one of embodiments 37-39, wherein said double-stranded RNA (dsRNA) duplex comprises at least 5, at least 8, at least 10, or at least 12 ribonucleotides.
41. A deoxyribonucleic acid polynucleotide encoding the engineered guide ribonucleic acid polynucleotide of any one of embodiments 1-40.
42. A nucleic acid comprising an engineered nucleic acid sequence optimized for expression in an organism, wherein said nucleic acid encodes a class 2, type V Cas endonuclease, and wherein said endonuclease is derived from an uncultivated microorganism, wherein the organism is not said uncultivated organism.
43. The nucleic acid of embodiment 42, wherein said endonuclease comprises a variant having at least 70% or at least 80% sequence identity to any one of SEQ ID NOs: 1-3470.
44. The nucleic acid of embodiment 42 or 43, wherein said endonuclease comprises a sequence encoding one or more nuclear localization sequences (NLSs) proximal to an N- or C-terminus of said endonuclease.
45. The nucleic acid of embodiment 44, wherein said NLS comprises a sequence selected from SEQ ID NOs: 3938-3953.
46. The nucleic acid of embodiment 44 or 45, wherein said NLS comprises SEQ ID NO: 3939.
47. The nucleic acid of embodiment 46, wherein said NLS is proximal to said N-terminus of said endonuclease.
48. The nucleic acid of embodiment 44 or 45, wherein said NLS comprises SEQ ID NO: 3938.
49. The nucleic acid of embodiment 48, wherein said NLS is proximal to said C-terminus of said endonuclease.
50. The nucleic acid of any one of embodiments 42-49, wherein said organism is prokaryotic, bacterial, eukaryotic, fungal, plant, mammalian, rodent, or human.
51. An engineered vector comprising a nucleic acid sequence encoding a class 2, type V Cas endonuclease, wherein said endonuclease is derived from an uncultivated microorganism.
52. An engineered vector comprising the nucleic acid of any of embodiments 42-46.
53. An engineered vector comprising the deoxyribonucleic acid polynucleotide of embodiment 41.
54. The engineered vector of any of embodiments 51-53, wherein the vector is a plasmid, a minicircle, a CELiD, an adeno-associated virus (AAV) derived virion, a lentivirus, or an adenovirus.
55. A cell comprising the vector of any of embodiments 51-54.
56. A method of manufacturing an endonuclease, comprising cultivating said cell of embodiment 55.
57. A method for binding, cleaving, marking, or modifying a double-stranded deoxyribonucleic acid polynucleotide, comprising:
   (a) contacting said double-stranded deoxyribonucleic acid polynucleotide with a class 2, type V Cas endonuclease in complex with an engineered guide RNA configured to bind to said endonuclease and said double-stranded deoxyribonucleic acid polynucleotide;
   wherein said double-stranded deoxyribonucleic acid polynucleotide comprises a protospacer adjacent motif (PAM); and
   wherein said PAM comprises a sequence comprising any one of Sequence Numbers: A3863-A3889, or any one of SEQ ID NOs: 3890-3913.
58. The method of embodiment 57, wherein said double-stranded deoxyribonucleic acid polynucleotide comprises a first strand comprising a sequence complementary to a sequence of said engineered guide RNA and a second strand comprising said PAM.
59. The method of embodiment 58, wherein said PAM is directly adjacent to the 5′ end of said sequence complementary to said sequence of said engineered guide RNA.
60. The method of any one of embodiments 57-59, wherein said PAM comprises a sequence of YYn.
61. The method of any one of embodiments 57-60, wherein said class 2, type V Cas endonuclease is derived from an uncultivated microorganism.
62. The method of any one of embodiments 57-61, wherein said double-stranded deoxyribonucleic acid polynucleotide is a eukaryotic, plant, fungal, mammalian, rodent, or human double-stranded deoxyribonucleic acid polynucleotide.
63. A method of modifying a target nucleic acid locus, said method comprising delivering to said target nucleic acid locus said engineered nuclease system of any one of embodiments 1-36, wherein said endonuclease is configured to form a complex with said engineered guide ribonucleic acid structure, and wherein said complex is configured such that upon binding of said complex to said target nucleic acid locus, said complex modifies said target nucleic acid locus.
64. The method of embodiment 63, wherein modifying said target nucleic acid locus comprises binding, nicking, cleaving, or marking said target nucleic acid locus.
65. The method of embodiment 63 or 64, wherein said target nucleic acid locus comprises deoxyribonucleic acid (DNA) or ribonucleic acid (RNA).
66. The method of embodiment 63, wherein said target nucleic acid comprises genomic DNA, viral DNA, viral RNA, or bacterial DNA.
67. The method of any one of embodiments 63-66, wherein said target nucleic acid locus is in vitro.
68. The method of any one of embodiments 63-66, wherein said target nucleic acid locus is within a cell.
69. The method of embodiment 68, wherein said cell is a prokaryotic cell, a bacterial cell, a eukaryotic cell, a fungal cell, a plant cell, an animal cell, a mammalian cell, a rodent cell, a primate cell, a human cell, or a primary cell.
70. The method of embodiment 68 or 69, wherein said cell is a primary cell.
71. The method of embodiment 70, wherein said primary cell is a T cell.
72. The method of embodiment 70, wherein said primary cell is a hematopoietic stem cell (HSC).
73. A method of any one of embodiments 63-72, wherein delivering said engineered nuclease system to said target nucleic acid locus comprises delivering the nucleic acid of any of embodiments 42-46 or the vector of any of embodiments 51-54.
74. The method of any one of embodiments 63-73, wherein delivering said engineered nuclease system to said target nucleic acid locus comprises delivering a nucleic acid comprising an open reading frame encoding said endonuclease.
75. The method of embodiment 74, wherein said nucleic acid comprises a promoter to which said open reading frame encoding said endonuclease is operably linked.
76. The method of any one of embodiments 63-75, wherein delivering said engineered nuclease system to said target nucleic acid locus comprises delivering a capped mRNA containing said open reading frame encoding said endonuclease.
77. The method of any one of embodiments 63-76, wherein delivering said engineered nuclease system to said target nucleic acid locus comprises delivering a translated polypeptide.
78. The method of any one of embodiments 63-76, wherein delivering said engineered nuclease system to said target nucleic acid locus comprises delivering a deoxyribonucleic acid (DNA) encoding said engineered guide RNA operably linked to a ribonucleic acid (RNA) pol III promoter.
79. The method of any one of embodiments 63-78, wherein said endonuclease induces a single-stranded break or a double-stranded break at or proximal to said target locus.
80. The method of embodiment 79, wherein said endonuclease induces a staggered single stranded break within or 3′ to said target locus.
81. A method of editing a TRAC locus in a cell, comprising contacting to said cell
   (a) an RNA-guided endonuclease; and
   (b) an engineered guide RNA, wherein said engineered guide RNA is configured to form a complex with said endonuclease and said engineered guide RNA comprises a spacer sequence configured to hybridize to a region of said TRAC locus,
      wherein said engineered guide RNA comprises a targeting sequence having at least 85% identity at least 18 consecutive nucleotides of any one of SEQ ID NOs: 4316-4369.
82. The method of embodiment 81, wherein said RNA-guided nuclease is a Cas endonuclease.
83. The method of embodiment 82, wherein said Cas endonuclease is a class 2, type V Cas endonuclease.
84. The method of embodiment 83, wherein said class 2, type V Cas endonuclease comprises a RuvC domain comprising a RuvCI subdomain, a RuvCII subdomain, and a RuvCIII subdomain.
85. The method of embodiment 83 or 84, wherein said class 2, type V Cas endonuclease comprises an endonuclease having at least 75% sequence identity to any one of SEQ ID NOs: 1-3470 or a variant thereof.
86. The method of any one of embodiments 81-85, wherein said engineered guide RNA further comprises a sequence with at least 80% sequence identity to at least 19 of the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, and 3851-3857.
87. The method of any one of embodiments 81-85, wherein said endonuclease comprises a sequence at least 75%, 80%, or 90% identical to any one of SEQ ID NOs: 141, 215, 229, 261, or 1711-1721 or a variant thereof.
88. The method of embodiment 87, wherein said guide RNA structure comprises a sequence at least 80%, or at least 90% identical to at least 19 of the non-degenerate nucleotides of any one of SEQ ID NOs: 3608-3609, 3853, or 3851-3857.
89. The method of any one of embodiments 81-88, wherein said method further comprises contacting to said cell or introducing to said cell a donor nucleic acid comprising a cargo sequence flanked on a 3′ or 5′ end by sequence having at least 80% identity to any one of SEQ ID NOs: 4424 or 4425.
90. The method of any one of embodiments 81-89, wherein said cell is a peripheral blood mononuclear cell (PBMC).
91. The method of any one of embodiments 81-89, wherein said cell is a T-cell or a precursor thereof or a hematopoietic stem cell (HSC).
92. The method of any one of embodiments 89-91, wherein said cargo sequence comprises a sequence encoding a T-cell receptor polypeptide, a CAR-T polypeptide, or a fragment or derivative thereof.
93. The method of any one of embodiments 81-92, wherein said engineered guide RNA comprises a sequence having at least 80% identity to any one of SEQ ID NOs:4370-4423.
94. The method of embodiment 93, wherein said engineered guide RNA is comprises the nucleotide sequence of sgRNAs 1-54 from Table 5A comprising the corresponding chemical modifications listed in Table 5A.
95. The method of any one of embodiments 81-93, wherein said engineered guide RNA comprises a targeting sequence having at least 80% sequence identity to any one of SEQ ID NOs: 4334, 4350, or 4324.
96. The method of any one of embodiments 81-93, wherein said engineered guide RNA comprises a sequence having at least 80% sequence identity to any one of SEQ ID NOs: 4388, 4404, or 4378.
97. The method of embodiment 96, wherein said engineered guide RNA comprises the nucleotide sequence of sgRNAs 9, 35, or 19 from Table 5A.
98. An engineered nuclease system comprising:
   (a) an RNA-guided endonuclease; and
   (b) an engineered guide RNA, wherein said engineered guide RNA is configured to form a complex with said endonuclease and said engineered guide RNA comprises a spacer sequence configured to hybridize to a target nucleic acid sequence,
   wherein said engineered guide RNA comprises at least one of the following modifications:
      (i) a 2′-O methyl or a 2′-fluoro base modification of at least one nucleotide within the first 4 bases of the 5′ end of said engineered guide RNA or the last 4 bases of a 3′ end of said engineered guide RNA;
      (ii) a thiophosphate (PS) linkage between at least 2 of the first five bases of a 5′ end of said engineered guide RNA, or a thiophosphate linkage between at least two of the last five bases of a 3′ end of said engineered guide RNA;
      (iii) a thiophosphate linkage within a 3′ stem or a 5′ stem of said engineered guide RNA;
      (iv) a 2′-O methyl or 2′base modification within a 3′ stem or a 5′ stem of said engineered guide RNA;
      (v) a 2′-fluoro base modification of at least 7 bases of a spacer region of said engineered guide RNA; and
      (vi) a thiophosphate linkage within a loop region of said engineered guide RNA.
99. The system of embodiment 98, wherein said engineered guide RNA comprises a 2′-O methyl or a 2′-fluoro base modification of at least one nucleotide within the first 5 bases of a 5′ end of said engineered guide RNA or the last 5 bases of a 3′ end of said engineered guide RNA.
100. The system of embodiment 98, wherein said engineered guide RNA comprises a 2′-O methyl or a 2′-fluoro base modification at a 5′ end of said engineered guide RNA or a 3′ end of said engineered guide RNA.
101. The system of any one of embodiments 98-100, wherein said engineered guide RNA comprises a thiophosphate (PS) linkage between at least 2 of the first five bases of a 5′ end of said engineered guide RNA, or a thiophosphate linkage between at least two of the last five bases of a 3′ end of said engineered guide RNA.
102. The system of any one of embodiments 98-101, wherein said engineered guide RNA comprises a thiophosphate linkage within a 3′ stem or a 5′ stem of said engineered guide RNA.
103. The system of any one of embodiments 98-102, wherein said engineered guide RNA comprises a 2′-O methyl base modification within a 3′ stem or a 5′ stem of said engineered guide RNA.
104. The system of any one of embodiments 98-103, wherein said engineered guide RNA comprises a 2′-fluoro base modification of at least 7 bases of a spacer region of said engineered guide RNA.
105. The system of any one of embodiments 98-104, wherein said engineered guide RNA comprises a thiophosphate linkage within a loop region of said engineered guide RNA.
106. The system of any one of embodiments 98-105, wherein said engineered guide RNA comprises at least three 2′-O methyl or 2′-fluoro bases at said 5′ end of said engineered guide RNA, two thiophosphate linkages between the first 3 bases of said 5′ end of said engineered guide RNA, at least 4 2′-O methyl or 2′-fluoro bases at said 4′ end of said engineered guide RNA, and three thiophosphate linkages between the last three bases of said 3′ end of said engineered guide RNA.
107. The system of embodiment 98, wherein said engineered guide RNA comprises at least two 2′-O-methyl bases and at least two thiophosphate linkages at a 5′ end of said engineered guide RNA and at least one 2′-O-methyl bases and at least one thiophosphate linkage at a 3′ end of said engineered guide RNA.
108. The system of embodiment 107, wherein said engineered guide RNA comprises at least one 2′-O-methyl base in both said 3′ stem or said 5′ stem region of said engineered guide RNA.
109. The system of embodiment 107 or 108, wherein said engineered guide RNA comprises at least one to at least fourteen 2′-fluoro bases in said spacer region excluding a seed region of said engineered guide RNA.
110. The system of embodiment 107, wherein said engineered guide RNA comprises at least one 2′-O-methyl base in said 5′ stem region of said engineered guide RNA and at least one to at least fourteen 2′-fluoro bases in said spacer region excluding a seed region of said guide RNA.
111. The system of any one of embodiments 98-110, wherein said guide RNA comprises a spacer sequence targeting a VEGF-A gene.
112. The system of embodiment 111, wherein said guide RNA comprises a spacer sequence having at least 80% identity to SEQ ID NO: 3985.
113. The system of embodiment 111, wherein said guide RNA comprises the nucleotides of guide RNAs 1-7 from Table 7 comprising the chemical modifications listed in Table 7.
114. The method of any one of embodiments 98-113, wherein said RNA-guided nuclease is a Cas endonuclease.
115. The method of embodiment 114, wherein said Cas endonuclease is a class 2, type V Cas endonuclease.
116. The method of embodiment 115, wherein said class 2, type V Cas endonuclease comprises a RuvC domain comprising a RuvCI subdomain, a RuvCII subdomain, and a RuvCIII subdomain.
117. The method of any one of embodiments 115-116, wherein said class 2, type V Cas endonuclease comprises an endonuclease having at least 75% sequence identity to any one of SEQ ID NOs: 1-3470 or a variant thereof.
118. The method of any one of embodiments 115-116, wherein said class 2, type V Cas endonuclease comprises an endonuclease having at least 75% sequence identity to any one of SEQ ID NOs: 141, 215, 229, 261, or 1711-1721 or a variant thereof.
119. The method of any one of embodiments 114-118, wherein said engineered guide RNA comprises a sequence with at least 80% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, and 3851-3857.
120. The method of any one of embodiments 111-118, wherein said engineered guide RNA comprises a sequence with at least 80% sequence identity to the non-degenerate nucleotides of any one of SEQ ID NOs: 3608-3609, 3853, or 3851-3857.
121. A host cell comprising an open reading frame encoding a heterologous endonuclease having at least 75% sequence identity to any one of SEQ ID NOs: 1-3470 or a variant thereof.
122. The host cell of embodiment 121, wherein said endonuclease has at least 75% sequence identity to any one of SEQ ID NOs: 141, 215, 229, 261, or 1711-1721, or a variant thereof.
123. The host cell of any one of embodiments 121-122 wherein said host cell is an E. coli cell.
124. The host cell of embodiment 123, wherein said E. coli cell is a DE3 lysogen or said E. coli cell is a BL21(DE3) strain.
125. The host cell of any one of embodiments 109-110, wherein said E. coli cell has an ompT Ion genotype.
126. The host cell of any one of embodiments 121-125, wherein said open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an αrαP_{BAD} promoter, a strong leftward promoter from phage lambda (μL promoter), or any combination thereof.
127. The host cell of any one of embodiments 121-126, wherein said open reading frame comprises a sequence encoding an affinity tag linked in-frame to a sequence encoding said endonuclease.
128. The method of embodiment 127, wherein said affinity tag is an immobilized metal affinity chromatography (IMAC) tag.
129. The method of embodiment 128, wherein said IMAC tag is a polyhistidine tag.
130. The method of embodiment 127, wherein said affinity tag is a myc tag, a human influenza hemagglutinin (HA) tag, a maltose binding protein (MBP) tag, a glutathione S-transferase (GST) tag, a streptavidin tag, a FLAG tag, or any combination thereof.
131. The host cell of any one of embodiments 127-130, wherein said affinity tag is linked in-frame to said sequence encoding said endonuclease via a linker sequence encoding a protease cleavage site.
132. The host cell of embodiment 131, wherein said protease cleavage site is a tobacco etch virus (TEV) protease cleavage site, a PreScission® protease cleavage site, a Thrombin cleavage site, a Factor Xa cleavage site, an enterokinase cleavage site, or any combination thereof.
133. The host cell of any one of embodiments 121-132, wherein said open reading frame is codon-optimized for expression in said host cell.
134. The host cell of any one of embodiments 121-133, wherein said open reading frame is provided on a vector.
135. The host cell of any one of embodiments 121-133, wherein said open reading frame is integrated into a genome of said host cell.
136. A culture comprising the host cell of any one of embodiments 121-135 in compatible liquid medium.
137. A method of producing an endonuclease, comprising cultivating the host cell of any one of embodiments 121-135 in compatible growth medium.
138. The method of embodiment 137, further comprising inducing expression of said endonuclease by addition of an additional chemical agent or an increased amount of a nutrient.
139. The method of embodiment 138, wherein an additional chemical agent or an increased amount of a nutrient comprises Isopropyl β-D-1-thiogalactopyranoside (IPTG) or additional amounts of lactose.
140. The method of any one of embodiments 137-139, further comprising isolating said host cell after said cultivation and lysing said host cell to produce a protein extract.
141. The method of embodiment 140, further comprising subjecting said protein extract to IMAC, or ion-affinity chromatography.
142. The method of embodiment 141, wherein said open reading frame comprises a sequence encoding an IMAC affinity tag linked in-frame to a sequence encoding said endonuclease.
143. The method of embodiment 142, wherein said IMAC affinity tag is linked in-frame to said sequence encoding said endonuclease via a linker sequence encoding protease cleavage site.
144. The method of embodiment 143, wherein said protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof.
145. The method of any one of embodiments 143-144, further comprising cleaving said IMAC affinity tag by contacting a protease corresponding to said protease cleavage site to said endonuclease.
146. The method of embodiment 145, further comprising performing subtractive IMAC affinity chromatography to remove said affinity tag from a composition comprising said endonuclease.
147. A system comprising
   (a) a class 2, Type V-A Cas endonuclease configured to bind a 3- or 4-nucleotide PAM sequence, wherein said endonuclease has increased cleavage activity relative to sMbCas12a; and
   (b) an engineered guide RNA, wherein said engineered guide RNA is configured to form a complex with said class 2, Type V-A Cas endonuclease and said engineered guide RNA comprises a spacer sequence configured to hybridize to a target nucleic acid comprising a target nucleic acid sequence.
148. The system of embodiment 147, wherein said cleavage activity is measured in vitro by introducing said endonucleases alongside compatible guide RNAs to cells comprising said target nucleic acid and detecting cleavage of said target nucleic acid sequence in said cells.
149. The system of any one of embodiments 147-148, wherein said class 2, Type V-A Cas endonuclease comprises a sequence having at least 75% identity to any one of 215-225 or a variant thereof.
150. The system of embodiment 149, wherein said engineered guide RNA comprises a sequence having at least 80% identity to the non-degenerate nucleotides of SEQ ID NO: 3609.
151. The system of any one of embodiments 149-150, wherein said target nucleic acid further comprises a YYN PAM sequence proximal to said target nucleic acid sequence.
152. The system of any one of embodiments 147-151, wherein said class 2, Type V-A Cas endonuclease has at least about 5%, 10%, 20%, 30%, 40%, 50%, 60%, 70%, 80%, 90%, 100%, or 200%, or more increased activity relative to sMbCas12a.
153. A system comprising:
   (a) a class 2, Type V-A′ Cas endonuclease; and
   (b) an engineered guide RNA, wherein said engineered guide RNA comprises a sequence having at least 80% identity to about 19 to about 25 or about 19 to about 31 consecutive nucleotides of a natural effector repeat sequence of a class 2, Type V Cas endonuclease.
154. The system of embodiment 153, wherein said natural effector repeat sequence is any one of SEQ ID NOs: 3560-3572.
155. The system of any one of embodiments 153-154, wherein said class 2, Type V-A′ Cas endonuclease has at least 75% identity to SEQ ID NO: 126.
156. A method of disrupting the VEGF-A locus in a cell, comprising introducing to said cell:
   (b) a class 2, type V Cas endonuclease; and
   (b) an engineered guide RNA, wherein said engineered guide RNA is configured to form a complex with said endonuclease and said engineered guide RNA comprises a spacer sequence configured to hybridize to a region of said VEGF-A locus,
      wherein said engineered guide RNA comprises a targeting sequence having at least 80% identity to SEQ ID NO: 3985; or
      wherein said engineered guide RNA comprises the nucleotide sequence of any one of guide RNAs 1-7 from Table 7.
157. The system of embodiment 156, wherein said class 2, type V Cas endonuclease comprises an endonuclease having at least 75% sequence identity to any one of SEQ ID NOs: 1-3470 or a variant thereof.
158. The system of any one of embodiments 156-157, wherein said class 2, type V Cas endonuclease comprises an endonuclease having at least 75% sequence identity to any one of SEQ ID NOs: 141, 215, 229, 261, or 1711-1721 or a variant thereof.
159. The system of any one of embodiments 156-158, wherein said engineered guide RNA comprises a sequence with at least 80% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, and 3851-3857.
160. The system of any one of embodiments 156-158, wherein said engineered guide RNA comprises a sequence with at least 80% sequence identity to the non-degenerate nucleotides of any one of SEQ ID NOs: 3608-3609, 3853, or 3851-3857.
161. A method of disrupting a locus in a cell, comprising contacting to said cell a composition comprising:
   (a) a class 2, type V Cas endonuclease having at least 75% identity to any one of SEQ ID NOs: 215-225 or a variant thereof; and
   (b) an engineered guide RNA, wherein said engineered guide RNA is configured to form a complex with said endonuclease and said engineered guide RNA comprises a spacer sequence configured to hybridize to a region of said locus,
   wherein said class 2, type V Cas endonuclease has at least equivalent cleavage activity to spCas9 in said cell.
162. The method of embodiment 161, wherein said cleavage activity is measured in vitro by introducing said endonucleases alongside compatible guide RNAs to cells comprising said target nucleic acid and detecting cleavage of said target nucleic acid sequence in said cells.
163. The method of any one of embodiments 161-162, wherein said composition comprises 20 pmoles or less of said class 2, type V Cas endonuclease.
164. The method of embodiment 163, wherein said composition comprises 1 pmol or less of said class 2, type V Cas endonuclease.

1.-160. (canceled)

161. A method of disrupting a TRAC locus in a cell, said method comprising introducing to said cell:
(a) a class 2, type V Cas endonuclease or a polynucleotide encoding said endonuclease, wherein said endonuclease comprises an amino acid sequence comprising at least 80% sequence identity to SEQ ID NO: 215; and
(b) an engineered guide ribonucleic acid or a polynucleotide encoding said engineered guide ribonucleic acid, wherein said engineered guide ribonucleic acid is configured to form a complex with said endonuclease, wherein said engineered guide ribonucleic acid comprises a spacer sequence configured to hybridize to a region of said TRAC locus, wherein said engineered guide ribonucleic acid comprises a nucleotide sequence having at least 80% sequence identity to any one of SEQ ID NOs: 5681, 5683, or 5056-5125.

162. The method of claim 161, wherein said engineered guide ribonucleic acid comprises a nucleotide sequence having at least 90% sequence identity to any one of SEQ ID NOs: 5681, 5683, or 5056-5125.

163. The method of claim 162, wherein said engineered guide ribonucleic acid comprises a nucleotide sequence of SEQ ID NOs: 5681, 5683, or 5056-5125.

164. The method of claim 163, wherein said engineered guide ribonucleic acid comprises a nucleotide sequence of SEQ ID NO: 5681.

165. The method of claim 164, wherein said endonuclease comprises a sequence of SEQ ID NO: 215.

166. The method of claim 161, wherein said region of said TRAC locus comprises a sequence having at least 90% sequence identity to at least 20-22 consecutive nucleotides of any one of SEQ ID NOs: 5682, 5684, or 5126-5195.

167. The method of claim 166, wherein said region of said TRAC locus comprises a sequence having at least 20-22 consecutive nucleotides of any one of SEQ ID NOs: 5682, 5684, or 5126-5195.

168. The method of claim 161, wherein said endonuclease is configured to be selective for a protospacer adjacent motif (PAM) sequence comprising 5′-TTTN-3′ of 5′-TTTG-3′.

169. The method of claim 161, wherein said endonuclease comprises a WED II domain and a PAM-interacting region.

170. The method of claim 169, wherein said WED II domain comprises an amino acid sequence having at least 80% sequence identity to a WED II domain of SEQ ID NO: 215.

171. The method of claim 170, wherein said WED II domain comprises an amino acid sequence having at least 80% sequence identity to amino acid residues 561-632 of SEQ ID NO: 215.

172. The method of claim 169, wherein said PAM-interacting region comprises an amino acid sequence having at least 80% sequence identity to a PAM-interacting region of SEQ ID NO: 215.

173. The method of claim 172, wherein said PAM-interacting region comprises an amino acid sequence having at least 80% sequence identity to amino acid residues 633-730 of SEQ ID NO: 215.

174. The method of claim 161, wherein said endonuclease comprises a RuvC domain comprising an amino acid sequence having at least 80% sequence identity to RuvCI, RuvCII, and RuvCIII domains of SEQ ID NO: 215.

175. The method of claim 161, wherein said endonuclease comprises one or more catalytic residues corresponding to residues G578-W579, K583, K641, D886, E976, or D1229 of SEQ ID NO: 215.

176. The method of claim 161, wherein said endonuclease comprises at least one of the following mutations: S168R, E172R, N577R, or Y170R when an amino acid sequence of said endonuclease is aligned to SEQ ID NO: 215.

177. The method of claim 161, wherein said endonuclease comprises an amino acid sequence having at least 90% sequence identity to SEQ ID NO: 215.

178. The method of claim 177, wherein said endonuclease comprises a sequence of SEQ ID NO: 215.

179. The method of claim 161, further comprising contacting said TRAC locus with a single- or double-stranded deoxyribonucleic acid repair template.

180. The method of claim 161, wherein said cell is a eukaryotic cell, a T-cell, a hematopoietic stem cell, or a precursor thereof.
